(19) United States
(12) Patent Application Publication
(10) Pub. No.: US 2024/0141434 A1
(43) Pub. Date: May 2, 2024

(54) METHODS FOR DETECTING AND PREDICTING BREAST CANCER

(71) Applicant: UCL BUSINESS LTD, London (GB)

(72) Inventors: Martin Widschwendter, London (GB); James Barrett, London (GB)

(21) Appl. No.: 18/009,936

(22) PCT Filed: Jun. 17, 2021
(86) PCT No.: PCT/GB2021/051540

§ 371 Date: Dec. 12, 2022

(30) Foreign Application Priority Data
Jun. 17, 2020 (GB) 2009220.1

(51) Int. Cl. C12Q 1/6886

(52) U.S. Cl. CPC C12Q 1/6886; C12Q 2600/118; C12Q 2600/154; G01N 2800/50

(57) ABSTRACT

The present invention relates to assays for predicting the presence, absence or development of cancer in an individual, particularly breast and ovarian cancer, by determining the methylation status of certain CpGs in a population of DNA molecules in a sample which has been taken from the individual, deriving an index value based on the methylation status of the certain CpGs, and predicting the presence, absence or development of cancer in the individual based on the cancer index value. The invention further relates to a method of treating and/or prevention of cancer in an individual, particularly breast and ovarian cancer, the method comprising assessing the presence, absence or development of cancer in an individual by performing the assays of the invention, followed by administering one or more therapeutic treatments or measures to the individual based on the assessment. The invention further provides a method of monitoring the cancer status of an individual according to changes in the individuals breast cancer index value over the course of time. The invention further relates to arrays which are suitable for performing the assays of the invention.

### SEQUENCE LISTING

The instant application contains a Sequence Listing which has been submitted electronically in ASCII format and is hereby incorporated by reference in its entirety. Said ASCII copy, created on Jun. 9, 2021, is named ‘Sequence listing as filed 17 Jun. 2021 N418732WO MGW JAS.txt’ and is 15,290,046 bytes in size.

### FIELD OF THE INVENTION

The present invention relates to assays for predicting the presence, absence or development of cancer in an individual, particularly breast and ovarian cancer, by determining the methylation status of certain CpGs in a population of DNA molecules in a sample which has been taken from the individual, deriving an index value based on the methylation status of the certain CpGs, and predicting the presence, absence or development of cancer in the individual based on the cancer index value. The invention further relates to a method of treating and/or preventing cancer in an individual, particularly breast and ovarian cancer, the method comprising assessing the presence, absence or development of cancer in an individual by performing the assays of the invention, followed by administering one or more therapeutic or preventative treatments or measures to the individual based on the assessment. The invention further provides a method of monitoring the cancer status of an individual according to changes in the individual's cancer index value over the course of time. The invention further relates to arrays which are suitable for performing the assays of the invention.

The project leading to this application has been funded by the European Commission's Horizon 2020 Research and Innovation Action, H2020 FORECEE under Grant Agreement No. 634570, the European Commission's Horizon 2020 European Research Council Executive Agency, H2020 BRCA-ERC under Grant Agreement No. 742432 as well as the charity, The Eve Appeal.

### BACKGROUND TO THE INVENTION

Breast cancer is by far the most common cancer in females in general, and a leading cause of death in young women. To date, the identification of individuals with primary cancer is achieved by assessing evidence directly from the tumour (e.g. imaging or detection of cancer cell products released into the system). Currently available early detection strategies, such as mammography screening, suffer from low performance in young women, over-diagnosis, and decreasing attendance rates, and its benefit on mortality rates has recently been questioned. Developing models which allow for a stratified breast cancer early detection and prevention strategy have proven to be challenging, and the best predictive models combining three different sources of information—epidemiological risk factors, single nucleotide polymorphisms (SNPs) and mammographic density—have only led to a Receiver Operator Characteristic (ROC) Area Under the Curve (AUC) of 0.68.

In contrast, cervical cancer screening (i.e. assessing cervical smear samples) has reduced the incidence and mortality from cervical cancer by more than 50%. The fact that clinician- and self-collected samples show similar performance in detecting relevant cervical lesions is likely to further increase attendance rates.

Epigenetic (i.e. DNAme) changes have been identified in normal breast tissue adjacent to breast cancers and could potentially serve as a surrogate for both genetic and non-genetic factors including lifestyle, reproductive and environmental exposures contributing to breast cancer development. A number of proof of principle studies, so far exclusively performed in blood, have demonstrated that certain DNAme changes are associated with breast cancer predisposition. Sample heterogeneity and the choice of surrogate tissue are deemed to be among the most important factors impeding clinical implementation.

Thus, the inventors aimed to assess whether DNAme profiles derived from cervical smear samples (i.e. containing hormone sensitive epithelial cells which are capable of recording breast cancer-predisposing factors at the level of the epigenome and can be self-collected) are able to identify women with primary breast cancer.

### SUMMARY OF THE INVENTION

The current inventors set out to understand whether DNAme (DNA methylation) profiles may be used to detect the presence or absence of cancer, particularly breast and ovarian cancer. The inventors also set out to understand whether said DNAme profiles may be associated with the development of cancer, and therefore whether such profiles may be capable of functioning as surrogate markers for individual stratification purposes in connection with cancer.

In this regard the inventors have succeeded in developing assays involving “cancer index values” which are derived from and associated with DNAme profiles established from samples from tissue comprising epithelial cells from a given individual. The sample may particularly be derived from the cervix, the vagina, the buccal area, blood and/or urine. The sample is preferably a cervical liquid-based cytology sample, and more preferably a cervical smear sample. Subsequent DNAme profiles from a given individual, and which values can be used to stratify the individual in connection with cancer. A preferred sample for use in any of the assays described and defined herein is a cervical liquid-based cytology sample. A particularly preferred sample for use in any of the assays described and defined herein is a cervical smear sample.

Accordingly, in contrast to prior art assays, the inventors have surprisingly determined that it is possible to derive “cancer index values” derived from and associated with DNAme profiles established from samples, wherein the samples are free of tumor-derived DNA. Thus the tissue(s) from which DNAme profiles of the present assays are established may act to provide surrogate markers for the presence, absence or development of cancer, wherein tumor cells, if present, or cells at risk of transforming into tumor cells, are located at an anatomical site distinct from the site from which the sample was taken.

The cancer index value is determined from data relating to the methylation status of one or more CpGs in a panel of CpGs as further defined and described herein. CpGs of the panel are methylation sites in DNA from cells derived from/obtained from a sample comprising epithelial cells. The sample may particularly be derived from the cervix, the vagina, the buccal area, blood and/or urine. The sample is preferably a cervical liquid-based cytology sample, and more preferably a cervical smear sample. Subsequent DNAme profiles from a given individual, and which values can be used to stratify the individual in connection with cancer. A preferred sample for use in any of the assays described and defined herein is a cervical liquid-based cytology sample. A particularly preferred sample for use in any of the assays described and defined herein is a cervical smear sample.

For the purposes of the present invention, the cancer index value may be used interchangeably herein with “WID-BC-Index”, “WID-Index”, “cancer index”, “index” or “index value” (WID=women's risk identification). Furthermore, any reference to a cancer index value in the context of the present invention, may be equally used for the assessment of the presence, absence or development of breast cancer and/or ovarian cancer in an individual.

Based on studies with patients known to be free of breast cancer, the inventors have established cancer index values, using specific panels of CpGs, which have been determined to be associated with/characteristic of breast tissue which is negative for breast cancer, i.e. normal breast tissue which is free of breast cancer. Based on studies with patients known to possess breast cancer, the inventors have established cancer index values which have been determined to be associated with/characteristic of breast tissue which is positive for breast cancer.

The inventors have further established that the same specific panels of CpGs that associate with breast tissue which is negative or positive for breast cancer may likewise be associated with ovarian tissue that is negative or positive for ovarian cancer. Based on studies with patients known to be free of ovarian cancer, the inventors have established cancer index values, using specific panels of CpGs, which have been determined to be associated with/characteristic of ovarian tissue which is negative for ovarian cancer, i.e. normal ovarian tissue which is free of ovarian cancer. Based on studies with patients known to possess ovarian cancer, the inventors have established cancer index values which have been determined to be associated with/characteristic of ovarian tissue which is positive for ovarian cancer.

Thus, the inventors have been able to establish cancer index values, using specific panels of CpGs, which can characterize an individual as having cancer or not having cancer, or having a high risk of cancer development.

By determining the methylation profile-based cancer index value from a sample derived from the individual, the individual may be seen to possess a cancer index value which correlates with those possessed by individuals which are known, via the inventor's studies described herein, to be cancer positive or negative. Such correlations have been determined with a high degree of statistical accuracy, particularly with respect to parameters relevant to biological assays such as receiver operating characteristics (ROC) sensitivity and specificity, as well as area under the curve (AUC). Accordingly, by determining the cancer index value from a sample from a given individual, the individual may be determined to possess breast and/or ovarian tissue which is positive for cancer, i.e. the individual is diagnosed as having breast and/or ovarian cancer. Conversely, by determining the cancer index value from a sample from a given individual, the individual may be determined to possess breast and/or ovarian tissue which is negative for cancer, i.e. the individual is diagnosed as not having breast and/or ovarian cancer.

By determining the methylation profile-based cancer index value from samples derived from women known to possess a BRCA1 and/or BRCA2 germline mutation, but are otherwise cancer negative, these women have an increased cancer index value relative to healthy women that do not possess a BRCA1 and/or BRCA2 germline mutation. Women with a BRCA1 and/or BRCA2 germline mutation are known to be at high risk of developing breast and/or ovarian cancer. Accordingly, an increased cancer index value in women that are, at the time of testing, cancer negative can indicate a high risk of said women developing cancer, particularly breast and/or ovarian cancer, most preferably breast cancer. Preventative therapies and intensified screening as described herein may be particularly suitable for these women that have a high risk of developing cancer, particularly breast and/or ovarian cancer, most preferably breast cancer.

The inventors have determined that the cancer index value is typically increased in individuals with BRCA1 and/or BRCA2 germline mutations. The inventors have also determined that the cancer index value increases in normal breast tissue surrounding a cancer lesion, increases further in the cancer lesion itself, and is particularly increased in a triple negative breast cancer lesion. This indicates that the breast cancer index value, determined in accordance with the present invention, is capable of identifying individuals that may be at risk of developing cancer, and are thereby pre-disposed to breast cancer. These observations from the current inventors further establish that the cancer index value, as further described and defined herein, is dynamic and can change according to genetic and environmental conditions including the grade and severity of the cancer. The cancer index value may therefore be used to monitor an individual's cancer status and risk of cancer development. Moreover, the cancer index value may be used to monitor the efficacy of cancer treatments being administered to an individual, including therapeutic treatments and preventative treatments.

Accordingly, in the context of the present invention, by determining the cancer index value from a sample from a given individual it is possible to assess the presence, absence or development of cancer in an individual, or in other words to stratify the individual for cancer. In the context of the present invention, stratification for cancer is the process of categorizing the individual as being a member of a group of individuals who possess a phenotype in connection with cancer, including the presence or absence of cancer in the individual, or the development of cancer, i.e. by having epithelial cells, particularly derived from an anatomical site other than the breast or ovary, such as the cervix, the vagina, the buccal area, blood and/or urine which is more characteristic of breast or ovarian tissue which is cancer positive than breast or ovarian tissue which is cancer negative. A preferred sample for use in any of the assays described and defined herein is a cervical liquid-based cytology sample. A particularly preferred sample for use in any of the assays described and defined herein is a cervical smear sample.

As explained herein, the assays methods of the invention are based on a cancer index value derived from a methylation profile from DNA originating from epithelial cells, particularly derived from an anatomical site other than the breast or ovary, such as the cervix, the vagina, the buccal area, blood and/or urine, preferably a cervical liquid-based cytology sample, more preferably a cervical smear sample. Accordingly, the assays provide means for correlating a cervical tissue, vaginal tissue, buccal tissue or cervicovaginal tissue-derived DNA methylation profile with a status connected with breast or ovarian cancer ranging from the individual being cancer negative, to the individual being cancer positive, with high statistical accuracy. Because the assays of the invention provide a correlation between the methylation profile and the disease status, the skilled person will appreciate that as part of the stratification process and outcome, disease status is assigned on the basis of a likelihood. As such, the methods of the invention provide assays which are predictive of an individual's status with respect to cancer. The assays of the invention accordingly provide means for predicting the presence or absence of cancer in an individual. The assays of the invention accordingly also provide means for predicting the development of cancer in an individual. The assays of the invention can provide means for predicting the development of cancer in an individual since the inventors have demonstrated that specific cancer index values can define breast and ovarian tissue which is cancer negative, whilst others can define breast and ovarian tissue which is cancer positive, and since the specific cancer index values may be dynamic and thereby increased in association with tumour grade and further increased cancer risk factors such as germline BRCA1 and/or BRCA2 mutation, the values may be subject to change along a scale of cancer risk.

Whilst disease status may be assigned on the basis of a likelihood, the inventors have demonstrated herein that correlations between DNA methylation profile and cancer status using cancer index values can be achieved with a very high degree of statistical accuracy using parameters relevant to biological assays, as described further herein. As such, the assays of the invention provide means for predicting the presence or absence of cancer in an individual and for predicting the development of cancer in an individual, and for stratifying an individual for cancer, and wherein the prediction/stratification can be defined to be statistically highly reliable and robust. This in turn means that the prediction/stratification can be made with a high level of confidence. The assays of the invention can be defined to be statistically accurate by means known in the art, as further described and defined herein. The assays of the invention can be defined according to parameters relating to their statistical specificity and sensitivity. These parameters define the likelihood of false positive and false negative test results. The lower the proportion of false positive and false negative test results the more statistically accurate the assay becomes. In this regard the inventors have established CpG panels, as described and defined further herein, wherein the methylation status of CpGs in the panel can be used to establish cancer index values such that the assays produce statistically accurate predictions of cancer status. Accordingly, the inventors have determined that the assays described herein may be defined according to statistical parameters such as percentage specificity and sensitivity and also by receiver operating characteristics (ROC) area under the curve (AUC). All such means are known in the art and are known to be defined measures of statistical accuracy for biological assays such as those described and defined herein.

Thus the methods of the invention provide assays which can be used, with a high degree of statistical accuracy, to predict the presence, absence or development of cancer. The methods of the invention provide assays which can be used, with a high degree of statistical accuracy, to stratify an individual with respect to cancer status. Accordingly, the methods of the invention provide useful information to individuals and their physicians concerning patient cancer status. This information may help inform actual therapeutic treatment measures if the presence of cancer is identified in the individual. The information may help to monitor the progress of therapeutic treatment measures in the individual by monitoring changes in the cancer index value over the course of a period of time. The information may help to monitor the progress of prophylactic or preventative treatment measures in the individual by monitoring changes in the cancer index value over the course of a period of time. As such the methods of the invention offer significant advantages in the personalised prevention and early detection as well as treatment and management of cancer in individuals.

Accordingly, the invention provides an assay for assessing the presence, absence or development of cancer in an individual, the assay comprising:
a. providing a sample which has been taken from the individual, the sample comprising a population of DNA molecules;
b. determining in the population of DNA molecules in the sample the methylation status of a panel of one or more CpGs selected from a panel of CpGs identified at nucleotide positions 61 to 62 in SEQ ID NOs 1 to 29,000;
c. deriving a cancer index value based on the methylation status of the one or more CpGs in the panel; and
d. assessing the presence, absence or development of cancer in the individual based on the cancer index value;
wherein the assay is characterised as having an area under the curve (AUC) of 0.60 or more as determined by receiver operating characteristics (ROC).

The assay of the invention may be performed above and additionally wherein the panel of one or more CpGs comprises at least 500 CpGs selected from the CpGs identified at nucleotide positions 61 to 62 in SEQ ID NOs 1 to 29,000, preferably wherein the assay is characterised as having an AUC of at least 0.60.

The assay of the invention may be performed above and additionally wherein the panel of one or more CpGs comprises at least the CpGs identified in SEQ ID NOs 1 to 500 and identified at nucleotide positions 61 to 62, preferably wherein the assay is characterised as having an AUC of at least 0.78.

The assay of the invention may be performed above and additionally wherein the panel of one or more CpGs comprises at least 1000 CpGs selected from the CpGs identified at nucleotide positions 61 to 62 in SEQ ID NOs 1 to 29,000, preferably wherein the assay is characterised as having an AUC of at least 0.61.

The assay of the invention may be performed above and additionally wherein the panel of one or more CpGs comprises at least the CpGs identified in SEQ ID NOs 1 to 1000 and identified at nucleotide positions 61 to 62, preferably wherein the assay is characterised as having an AUC of at least 0.81.

The assay of the invention may be performed above and additionally wherein the panel of one or more CpGs comprises at least 2000 CpGs selected from the CpGs identified at nucleotide positions 61 to 62 in SEQ ID NOs 1 to 29,000, preferably wherein the assay is characterised as having an AUC of at least 0.61.

The assay of the invention may be performed above and additionally wherein the panel of one or more CpGs comprises at least the CpGs identified in SEQ ID NOs 1 to 2000 and identified at nucleotide positions 61 to 62, preferably wherein the assay is characterised as having an AUC of at least 0.82.

The assay of the invention may be performed above and additionally wherein the panel of one or more CpGs comprises at least 10,000 CpGs selected from the CpGs identified at nucleotide positions 61 to 62 in SEQ ID NOs 1 to 29,000, preferably wherein the assay is characterised as having an AUC of at least 0.62.

The assay of the invention may be performed above and additionally wherein the panel of one or more CpGs comprises at least the CpGs identified in SEQ ID NOs 1 to 10,000 and identified at nucleotide positions 61 to 62, preferably wherein the assay is characterised as having an AUC of at least 0.84.

The assay of the invention may be performed above and additionally wherein the panel of one or more CpGs comprises at least the 29,000 CpGs identified at nucleotide positions 61 to 62 in SEQ ID NOs 1 to 29,000, and further wherein the assay is characterised as having an AUC of at least 0.84.

The assay of the invention may be performed above and additionally wherein the step of determining in the population of DNA molecules in the sample the methylation status of the one or more CpGs in the panel further comprises determining a β value of each CpG.

The assay of the invention may be performed above and additionally wherein the step of deriving the cancer index value based on the methylation status of the one or more CpGs in the panel comprises:
a. providing a methylation β-value data set comprising the methylation β-values for each CpG in the panel; and
b. providing a mathematical model capable of generating the cancer index from the methylation β-value data set; and
c. applying the mathematical model to the methylation β-value data set, thereby generating the cancer index.

The assay of the invention may be performed above and additionally wherein the cancer index value is a breast cancer index value (WID-BC-Index), and wherein the mathematical model which is applied to the methylation β-value data set to generate the cancer index is an algorithm according to the following formula:

$$WID - BC - index = {\sum }_{i = 1}^{n} \frac{μ - {w}_{i} {β}_{i}}{σ}$$

wherein:
   a. β₁, . . . , βₙ are methylation beta-values (between 0 and 1);
   b. w₁, . . . , w_{29,000} are real valued coefficients;
   c. μ and σ are real valued parameters used to scale the index; and
   d. n refers to the number of CpGs in the panel of one or more CpGs;

preferably wherein the cancer is breast cancer.

The assay of the invention may be performed above and additionally wherein when the cancer index value for the individual is about −0.278 or more, the individual is assessed as having cancer or as having high risk of cancer development, or wherein when the cancer index value for the individual is less than about −0.278, the individual is assessed as not having cancer or as having a low risk of cancer development, preferably wherein:
a. the panel of one or more CpGs comprises at least 500 of the CpGs identified at nucleotide positions 61 to 62 in SEQ ID NOs 1 to 29,000, and wherein the sensitivity is at least 63% and the specificity is at least 21%;
b. the panel of one or more CpGs comprises at least the CpGs defined by SEQ ID NOs 1 to 500 and identified at nucleotide positions 61 to 62, and wherein the sensitivity is at least 87% and specificity is at least 50%;
c. the panel of one or more CpGs comprises at least the CpGs defined by SEQ ID NOs 1 to 1000 and identified at nucleotide positions 61 to 62, and wherein the sensitivity is at least 89% and specificity is at least 49%; or
d. the panel of one or more CpGs comprises at least the CpGs defined by SEQ ID NOs 1 to 2000 and identified at nucleotide positions 61 to 62, and wherein the sensitivity is at least 89% and specificity is at least 48%;
preferably wherein the assay comprises determining methylation β-values for each CpG in the panel of one or more CpGs, more preferably wherein the cancer is breast cancer.

The assay of the invention may be performed above and additionally wherein when the cancer index value for the individual is about 0.126 or more, the individual is assessed as having cancer or as having a high risk of cancer development, or wherein when the cancer index value for the individual is less than about 0.126, the individual is assessed as not having cancer or as having a low risk of cancer development, preferably wherein:
a. the panel of one or more CpGs comprises at least 500 of the CpGs identified at nucleotide positions 61 to 62 in SEQ ID NOs 1 to 29,000, and wherein the sensitivity is at least 48% and the specificity is at least 39%;
b. the panel of one or more CpGs comprises at least the CpGs defined by SEQ ID NOs 1 to 500 and identified at nucleotide positions 61 to 62, and wherein the sensitivity is at least 70% and specificity is at least 72%;
c. the pane of one or more CpGs comprises at least the CpGs defined by SEQ ID NOs 1 to 1000 and identified at nucleotide positions 61 to 62, and wherein the sensitivity is at least 70% and specificity is at least 78%; or
d. the panel of one or more CpGs comprises at least the CpGs defined by SEQ ID NOs 1 to 2000 and identified at nucleotide positions 61 to 62, and wherein the sensitivity is at least 74% and specificity is at least 78%;
preferably wherein the assay comprises determining methylation β-values for each CpG in the panel of one or more CpGs, more preferably wherein the cancer is breast cancer.

The assay of the invention may be performed above and additionally wherein when the cancer index value for the individual is about 0.743 or more, the individual is assessed as having cancer or as having a high risk of cancer development, or wherein when the cancer index value for the individual is less than about 0.743, the individual is assessed as not having cancer or as having a low risk of cancer development, preferably wherein:
a. the panel of one or more CpGs comprises at least 500 of the CpGs identified at nucleotide positions 61 to 62 in SEQ ID NOs 1 to 29,000, and wherein the sensitivity is at least 17% and the specificity is at least 77%;
b. the panel of one or more CpGs comprises at least the CpGs defined by SEQ ID NOs 1 to 500 and identified at nucleotide positions 61 to 62, and wherein the sensitivity is at least 30% and specificity is at least 95%;
c. the panel of one or more CpGs comprises at least the CpGs defined by SEQ ID NOs 1 to 1000 and identified at nucleotide positions 61 to 62, and wherein the sensitivity is at least 31% and specificity is at least 96%; or
d. the panel of one or more CpGs comprises at least the CpGs defined by SEQ ID NOs 1 to 2000 and identified at nucleotide positions 61 to 62, and wherein the sensitivity is at least 29% and specificity is at least 96%; preferably wherein the assay comprises determining methylation β-values for each CpG in the panel of one or more CpGs, more preferably wherein the cancer is breast cancer.

The assay of the invention may be performed above and additionally wherein when the cancer index value for the individual is:
a. less than about −0.580 the individual is assessed as not having cancer;
b. about −0.580 or more and less than about −0.280 the individual is assessed as having a low risk of cancer;
c. about −0.280 or more and less than about 0.070 the individual is assessed as having a moderate risk of cancer;
d. about 0.070 or more the individual is assessed as having a high risk of cancer;
   preferably wherein the assay comprises determining methylation β-values for each CpG in the panel of one or more CpGs, more preferably wherein the cancer is breast cancer.

The assay of the invention may be performed above and additionally wherein the step of determining in the population of DNA molecules in the sample the methylation status of each CpG in the panel of one or more CpGs comprises:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

The assay of the invention may be performed above and additionally wherein the step of determining the methylation status of each CpG in the panel of one or more CpGs comprises:
a. bisulphite converting the DNA; or
b. performing the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

The assay of the invention may be performed above and additionally wherein the assay further comprises:
a. determining in the sample from the individual the proportion of epithelial cells;
b. determining in the sample from the individual the proportion of fat cells; and/or
c. determining in the sample from the individual differentiation characteristics of non-fat cells.

The assay of the invention may be performed above and additionally wherein determining the proportion of epithelial and/or fat cells and/or determining differentiation characteristics of non-fat cells comprises performing a method comprising:
a. gene expression profiling;
b. non-coding RNA-profiling;
c. epigenome profiling;
d. DNA methylation profiling;
e. deriving a WID-BC-Index; and/or
f. immunohistochemistry; and

arriving at a determination based on the results of the method.

The invention also provides a method of treating or preventing cancer in an individual, the method comprising:
a. assessing the cancer status of the individual by assessing the presence, absence or development of cancer in the individual by performing the assay of any one of the assays of the invention;
b. administering one or more therapeutic or preventative treatments to the individual based on the assessment.

The method of the invention may be performed above and additionally wherein the individual is assessed as not having cancer or as having a low risk of cancer development, and wherein when the cancer index value is about −0.580 or more and less than about −0.280, and preferably wherein the assay comprises determining methylation β-values for each CpG in the panel of one or more CpGs, the individual is subjected to one or more treatments according to their cancer index value, wherein the one or more treatments comprise intensified screening, preferably wherein the intensified screening comprises any of:
a. a test for a BRCA1 and/or BRCA2 germline mutation;
b. a repeat assay according to any one of the assays of the invention, preferably wherein the repeat assay is about two years after the previous assay.

The method of the invention may be performed above and additionally wherein when the test for a BRCA1 and/or BRCA2 germline mutation is positive, the intensified screening further comprises a mammogram.

The method of the invention may be performed above and additionally wherein when the test for a BRCA1 and/or BRCA2 germline mutation is negative, the individual is subjected to routine mammography screening, preferably wherein the routine screening comprises a mammogram about three years following the test for a BRCA1 and/or BRCA2 germline mutation.

The method of the invention may be performed above and additionally wherein the individual is assessed as having a moderate risk of having cancer or as having a moderate risk of cancer development, and wherein when the cancer index value is about −0.280 or more and less than about 0.070, and preferably wherein the assay comprises determining methylation β-values for each CpG in the panel of one or more CpGs, the individual is subjected to one or more treatments according to their cancer index value, wherein the one or more treatments comprise any of:
a. intensified screening, preferably wherein the intensified screening comprises one or more of:
   i. a test for a BRCA1 and/or BRCA2 germline mutation;
   ii. a breast MRI scan, preferably wherein the scan is repeated about two years after the previous scan;
   iii. a repeat assay according to any one of the assays of the invention, preferably wherein the repeat assay is performed about one year after the previous assay;
b. administration of one or more of Denosumab, “selective estrogen receptor modulators” (SERMs), and “selective progesterone receptor modulators” (SPRMs).

The method of the invention may be performed above and additionally wherein the individual is assessed as having cancer or as having a high risk of cancer development, and wherein when the cancer index value is about 0.070 or more, and preferably wherein the assay comprises determining methylation β-values for each CpG in the panel of one or more CpGs, and the individual is subjected to one or more treatments according to their cancer index value, wherein the one or more treatments comprise any of:
a. intensified screening, preferably wherein the intensified screening comprises one or more of:
   i. a test for a BRCA1 and/or BRCA2 germline mutation;
   ii. a breast MRI scan, preferably wherein the scan is repeated about a year after the previous scan;
   iii. a mammogram, preferably wherein the mammogram is repeated about a year after the previous mammogram;
   iv. a test for CA125, preferably wherein the test is repeated three-monthly;
   v. a test for cell-free tumour DNA methylation in plasma/serum, preferably wherein the test is repeated annually;
   vi. a test for cell-free tumour DNA methylation in vaginal fluid, preferably wherein the test is repeated annually;
   vii. a repeat assay according to any one of the assays of the invention, preferably wherein the repeat assay is performed about one year after the previous assay;
b. administration of one or more of Denosumab, “selective estrogen receptor modulators” (SERMs), and “selective progesterone receptor modulators” (SPRMs);
c. a bilateral mastectomy; and/or
d. a bilateral salpingo-oophorectomy.

The method of the invention may be performed above and additionally wherein the one or more treatments that the individual is subjected to are repeated on a monthly, three monthly, six monthly, yearly or two yearly basis following an initial administration.

The method of the invention may be performed above and additionally wherein:
a. the SERMs comprise Anordin, Bazedoxifene, Broparestrol, Broparestrol, Clomifene, Cyclofenil, Lasofoxifene, Ormeloxifene, Ospemifene, Raloxifene, Tamoxifen, preferably wherein the SERMs comprise Tamoxifen, Bazedoxifene and Raloxifene; and/or
b. the SPRMs comprise Mifepristone, Ulipristal, Asoprisnil, Proellex, Onapristone, Asoprisnil and Lonaprisan.

The invention also provides a method of monitoring the cancer status of an individual according to the individual's cancer index value, the method comprising: (a) assessing the presence, absence or development of cancer in an individual by performing the assay according to any one of the assays of the invention at a first time point; (b) assessing the presence, absence or development of cancer in the individual by performing the assay according to any one of the assays of the invention at one or more further time points; and (c) monitoring any change in the cancer status of the individual between time points.

The method of the invention may be performed above and additionally wherein the further time points are monthly, three monthly, six monthly, yearly or two yearly basis following an initial assessment.

The method of the invention may be performed above and additionally wherein depending on the cancer status of the individual, one or more treatments are administered to the individual according to any one of the methods of the invention, or wherein when the cancer the cancer index value of the individual is less than about −0.580 no treatment is administered to the individual.

The method of the invention may be performed above and additionally wherein an increase in the cancer index value indicates a negative response to the one or more treatments.

The method of the invention may be performed above and additionally wherein changes are made to the one or more treatments if a negative response is identified.

The method of the invention may be performed above and additionally wherein a decrease in the cancer index value indicates a positive response to the one or more treatments.

The method of the invention may be performed above and additionally wherein changes are made to the one or more treatments if a positive response is identified.

The method of the invention may be performed above and additionally wherein the method further comprises:
a. determining the proportion of epithelial cells in the sample from the individual between any two or more time points and assessing if the proportion changes between time points;
b. determining the proportion of fat cells in the sample from the individual between any two or more time points and assessing if the proportion changes between time points; and/or
c. determining differentiation characteristics of non-fat cells in the sample from the individual between any two or more time points and assessing if the proportion changes between time points.

The method of the invention may be performed above and additionally wherein:
a. an increase in the cancer index value and an increase in the proportion of epithelial cells; and/or
b. an increase in the cancer index value and a decrease in the proportion of fat cells; and/or
c. an increase in the cancer index value and an increase in differentiation of non-fat cells towards fat cells, indicates a negative response to the one or more treatments.

The method of the invention may be performed above and additionally wherein changes are made to the one or more treatments if a negative response is identified.

The method of the invention may be performed above and additionally wherein:
a. a decrease in the cancer index value and a decrease in the proportion of epithelial cells;
b. a decrease in the cancer index value and an increase in the proportion of fat cells; and/or
c. a decrease in the cancer index value and a decrease in differentiation of non-fat cells towards fat cells, indicates a positive response to the one or more treatments.

The method of the invention may be performed above and additionally wherein changes are made to the one or more treatments if a positive response is identified.

The assay of the invention may be performed above and additionally wherein the sample is obtained from a tissue comprising epithelial cells, preferably wherein the sample is not obtained from breast or ovarian tissue.

The assay of the invention may be performed above and additionally wherein the sample is obtained from:
a. cervical tissue;
b. vaginal tissue;
c. cervicovaginal tissue;
d. buccal tissue; and/or
e. breast tissue;
   preferably wherein the sample is obtained from cervical tissue, most preferably wherein the sample is obtained from tissue from a cervical smear.

The assay of the invention may be performed above and additionally wherein the assay is for assessing the presence, absence or development of:
a. breast cancer, particularly wherein the breast cancer is ductal carcinoma in situ; an invasive ductal carcinoma such as tubular type invasive ductal carcinoma (IDC), medullary type IDC, mucinous type IDC, papillary type IDC cribriform type IDC; invasive lobular carcinoma, inflammatory breast cancer, lobular carcinoma in situ, male breast cancer, luminal A breast cancer, luminal B breast cancer, triple-negative/basal-like breast cancer, HER2-enriched breast cancer, normal-like breast cancer, Paget's Disease of the nipple, Phyllodes tumours of the breast, or metastatic breast cancer;
b. ovarian cancer, particularly wherein the ovarian cancer is serious carcinoma, mucinous carcinoma, endometrioid carcinoma, clear cell carcinoma, low malignant potential (LMP) tumor, borderline epithelial ovarian cancer, teratoma, dysgerminoma, endodermal sinus tumor, Choriocarcinoma, granulosa-theca tumor, Sertoli-Leydig tumor, granulosa cell tumor, small cell carcinoma of the ovary or primary peritoneal carcinoma.

The invention also provides an array capable of discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel consists of at least 500 CpGs selected from the CpGs identified at nucleotide position 61 to 62 in SEQ ID NOs 1 to 29,000.

The array of the invention may be performed above and additionally provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less.

The array of the invention may be performed above and additionally wherein the panel comprises any set of CpGs defined in the assays of any one of the assays of the invention.

The array of the invention may be performed above and additionally further comprising one or more oligonucleotides comprising any set of CpGs defined in the assays of the invention, wherein the one or more oligonucleotides are hybridized to corresponding oligonucleotide probes of the array.

The invention also provides a hybridized array, wherein the array is obtainable by hybridizing to an array according to any one of the arrays of the invention a group of oligonucleotides comprising any set of CpGs defined in the assays of the invention. The invention also provides a process for making the hybridized array according to the invention, comprising contacting an array according to the arrays of the invention with a group of oligonucleotides comprising any set of CpGs defined in the assays of any one of the assays of the invention.

### BRIEF DESCRIPTION OF THE FIGURES

FIG. 1 shows training of the WID-BC-index classifiers and predictive performance of the WID-BC index, wherein optionally the WID-BC-index controls for immune cell proportion.

(A) Distribution of different cell types in the discovery dataset inferred using the HEpiDISH algorithm (* p<0.05, ** p<0.01, *** p<0.001). (B) Example of a CpG with cell-type specific methylation. (C) Area under the receiver operating characteristic curve (AUROC) in the internal validation set as a function of the number of CpGs used to train the classifier. (D) ROC curves of the WID-BC-index in the internal validation set for samples with an immune cell (IC) proportion ≤0.5 and >0.5. (E) Distribution of the WID-BC-index with respect to immune cell proportion in the internal validation set. (F) Distribution of the estimated variance in epithelial and immune cells across all CpGs used in the WID-BC-index. (G) The proportion of variance, weighted by the absolute coefficient values from the WID-BC-index, corresponding to each component of the index. (H) Performance of the WID-BC-index in the internal validation set after omitting each subcomponent. (I) Odds ratios corresponding to the genomic annotation of the 29,000 CpGs comprising the WID-BC-index (when compared to the 776,725 CpGs in the dataset; * p<0.05, ** p<0.01, *** p<0.001). (J) AUC values in the internal validation set after training classifiers on different subsets of the CpGs used in the WID-BC-index. The top n CpGs were either retained or removed. CpGs were also split into separate bins of size 500.

FIG. 2 shows external validation of the WID-BC-index, performance of the WID-BC-index in discriminating cancer-free women and women with endometrial cancer, and association between the WID-BC-index and germline BRCA1 mutation.

(A) WID-BC-index versus immune cell proportion in the external validation set. (B) ROC curve in the external validation set. (C-E) Performance of the WID-BC-index discriminating BRCA1 carriers from healthy controls in cervical, buccal and blood samples respectively.

FIG. 3 shows WID-BC-index performance when using buccal samples and association of WID-BC-index with ovarian cancer.

(A) WID-BC-index versus immune cell proportion in matched buccal samples from the breast cancer internal validation set. (B) ROC curve corresponding to the matched buccal samples. (C) WID-BC-index versus immune cell proportion in ovarian cancer cases and healthy controls. (D) ROC curve corresponding to the ovarian cancer samples.

FIG. 4 shows WID-BC-index data and its association with clinically relevant factors.

(A) The WID-BC-index versus age in samples from the internal and external validation datasets. (B) Correlation with a polygenic risk score (PRS) based on 303 SNPs in the internal validation dataset. (C) ROC curve corresponding to the PRS evaluated in a subset of samples from the discovery set. (D) Correlation with body mass index (BMI) in controls. (E) Family history. (F) Age at menarche. (G) Age at first live birth. (H) Menopause. (I) Hormone replacement therapy, (J) Stage, (K) Nodal status, (L) Grade. Panels (D) to (I) are based on controls from the internal and external validation datasets. Panels (J) to (L) are based on cases from the internal and external validation datasets.

FIG. 5 shows WID-BC-index data and its association with tumour cell fraction, known cancer markers, indicators of cell type of origin, and cervical microbiome type.

(A) The WID-BC-index evaluated in ENCODE primary cells (pc) and in vitro differentiated cells (ivdc). (B) The WID-index evaluated in ENCODE tissue samples. (C) The WID-BC-index evaluated in breast tissue samples. (D) WID-BC-index after linear adjustment for fat content in breast tissue samples from healthy women, women with BRCA mutation, triple negative breast cancers (TNBC) and matched adjacent normal tissue. (E) Top ten tissue-specific patterns enriched in hyper-methylated CpGs inferred using eFORGE. (F) Top ten tissue-specific patterns enriched in hypo-methylated CpGs. (G) The first two principal components corresponding to the bimodal CpGs in samples with IC<0.5. Samples are labelled with their corresponding cervicovaginal microbiome type, Type-L (Lactobacilli dominating) or Type-O (Non-lactobacilli dominating).

FIG. 6 shows the experimental design underpinning the discovery and validation of the WID-BC-index.

FIG. 7 shows cell type distributions in different datasets.

Distribution of different cell-types as estimated using the HEpiDISH algorithm in (A) the external validation dataset, (B) BRCA1 buccal samples, (C) BRCA2 buccal samples, (D) BRCA1 cervical samples, (E) BRCA2 cervical samples, (F) ovarian cancer samples, (G) matched buccal samples from the breast cancer internal validation dataset, (H) fresh breast tissue samples.

FIG. 8 shows the performance of the WID-BC-index in the discriminatory performance for assessing ovarian cancer risk of BRCA2 germline mutation carriers.

(A) Bimodal, (B) Epithelial, (C) Immune, and (D) Shared subcomponents of the WID-BC-index in the internal validation dataset. ROC curves corresponding to the WID-BC-index discriminating between BRCA2 carriers and healthy controls in (E) cervical, (F) buccal, and (G) blood samples.

FIG. 9 shows correlation of WID-BC-index derived from different sample tissues.

Correlations between (A) the overall WID-BC-index, and the (B) bimodal, (C) epithelial, (D) immune, and (E) shared subcomponents between matched cervical and blood samples from BRCA carriers and healthy controls. Correlations between (F) overall index, and (G) bimodal, (H) epithelial, (I) immune, and (J) shared subcomponents in matched cervical and buccal samples from the BRCA dataset. Correlations between (K) the overall index, and (L) bimodal, (M) epithelial, (N) immune, and (O) shared subcomponents in matched buccal and blood samples from the BRCA dataset.

FIG. 10 shows WID-BC-index performance of buccal samples and ovarianc cancer subcomponents.

(A) Bimodal, (B) Epithelial, (C) Immune, and (D) Shared subcomponents of the WID-BC-index in matched buccal samples from the breast cancer internal validation dataset. (E) Bimodal, (F) Epithelial, (G) Immune, and (H) Shared subcomponents of the WID-BC-index in ovarian cancer cases and healthy controls.

FIG. 11 shows a lack of association of WID-BC-index with other technical parameters.

(A) The WID-BC-index is independent of the time between sample collection and processing (Discovery set). (B) Distribution of the WID-BC-index in controls that volunteered from the general population and women that presented at hospitals for benign women-specific conditions (Discovery set).

FIG. 12 shows association between WID-BC-index components determined by IC fraction and cervicovaginal microbiome.

(A) The first two principal components corresponding to the CpGs comprising the epithelial subcomponent of the WID-BC-index in samples with IC<0.5. Samples are labelled with their corresponding cervicovaginal microbiome type. (B) First two principal components corresponding to the immune subcomponent in samples with IC>0.5. (C) First two principal components corresponding to the shared subcomponent in all samples.

FIG. 13 shows cutpoints applied to the patient data, and consequent specificity and sensitivity for cancer status discrimination achieved when these cutpoints are applied.

### DETAILED DESCRIPTION OF THE INVENTION

### Identification of CpGs

The present inventors sought to identify CpG methylation-based assays capable of assessing the presence, absence or development of cancer in an individual. Any of the assays described herein for assessing the presence, absence or development of cancer in an individual are capable of being utilised for assessing the presence, absence or development of breast cancer and/or ovarian cancer. The present inventors compared CpG methylation levels in non-cancerous epithelial cells, particularly derived from an anatomical site other than the breast or ovary, such as the cervix, the vagina, the buccal area, blood and/or urine, preferably cervical liquid-based cytology sample and more preferably derived from a cervical smear sample, across groups of women that were either known to be both breast and/or ovarian cancer negative, or known to be breast and/or ovarian cancer positive, or known to have a BRCA1 and/or BRCA2 germline mutation but are as yet breast and ovarian cancer free. This led to the surprising establishment of a “cancer index”, used interchangeable herein with “index”, “index value”, “WID-BC-Index” or “WID-Index” (WID=women's risk identification).

A CpG as defined herein refers to the CG dinucleotide motif identified in relation to each SEQ ID NO., wherein the CG dinucleotide of interest is denoted by [CG]. Thus by determining the methylation status of the cytosine of the CG dinucleotide motif identified at positions 61 to 62 in the panel of one or more CpGs of SEQ ID NOs 1 to 29,000, accepting that variations in the sequence upstream and downstream of any given CpG, as identified at positions 61 to 62 of any given SEQ ID NO, may exist due to sequencing errors or variation between individuals.

As set out in more detail in the Examples, the methylation status of sub-selections of the 29,000 CpGs, as identified in SEQ ID NOs 1 to 29,000, may be determined in order to assess an individual for the presence, absence or development of cancer with high sensitivity and specificity. A panel of one or more of the CpGs identified in SEQ ID NOs 1 to 29,000 may be utilised to derive a cancer index for an individual in accordance with the invention described herein.

The methylation status of a panel of one or more CpGs of the 29,000 CpGs defined according to SEQ ID NOs: 1 to 29,000 may be assessed by any suitable technique. As explained in more detail in the Examples below, one particular exemplary technique which the inventors have used is an array-based analysis technique coupled with beta value analysis. SEQ ID NOs 1 to 29,000 correspond to the sequences of commercial probes utilised in said array.

### Cancer-Related CpGs for Analysis

In any of the assays described herein, in a sample which has been taken from an individual, the sample comprises a population of DNA molecules.

The assay of the invention further comprises determining in the population of DNA molecules in the sample the methylation status of a panel of one or more CpGs selected from a panel of CpGs identified at nucleotide positions 61 to 62 in SEQ ID NOs 1 to 29,000. A cancer index value is then derived based on the methylation status of the one or more CpGs in the panel, which is used to assess the presence, absence or development of cancer in the individual based on the cancer index value.

In any of the assays described herein, in DNA derived from cells in the sample the methylation status of each CpG in a panel of one or more CpGs from a panel of CpGs identified at nucleotide positions 61 to 62 in SEQ ID NOs 1 to 29,000, is determined.

In any of the assays described herein, the panel of one or more CpGs may comprise at least 500 CpGs selected from the CpGs identified at nucleotide positions 61 to 62 in SEQ ID NOs 1 to 29,000, preferably wherein the assay is characterised as having a receiver operating characteristics (ROC) area under the curve (AUC) of at least 0.60. The panel of one or more CpGs may comprise at least the CpGs identified in SEQ ID NOs 1 to 500 and identified at nucleotide positions 61 to 62, preferably wherein the assay is characterised as having an ROC AUC of at least 0.78.

In any of the assays described herein, the panel of one or more CpGs may comprise at least 1000 CpGs selected from the CpGs identified at nucleotide positions 61 to 62 in SEQ ID NOs 1 to 29,000, preferably wherein the assay is characterised as having a ROC AUC of at least 0.61. The panel of one or more CpGs may comprise at least the CpGs identified in SEQ ID NOs 1 to 1000 and identified at nucleotide positions 61 to 62, preferably wherein the assay is characterised as having a ROC AUC of at least 0.81.

In any of the assays described herein, the panel of one or more CpGs may comprise at least 2000 CpGs selected from the CpGs identified at nucleotide positions 61 to 62 in SEQ ID NOs 1 to 29,000, preferably wherein the assay is characterised as having a ROC AUC of at least 0.61. The panel of one or more CpGs may comprise at least the CpGs identified in SEQ ID NOs 1 to 2000 and identified at nucleotide positions 61 to 62, preferably wherein the assay is characterised as having a ROC AUC of at least 0.82.

In any of the assays described herein, the panel of one or more CpGs may comprise at least 10,000 CpGs selected from the CpGs identified at nucleotide positions 61 to 62 in SEQ ID NOs 1 to 29,000, preferably wherein the assay is characterised as having an AUC of at least 0.62. The panel of one or more CpGs may comprise at least the CpGs identified in SEQ ID NOs 1 to 10,000 and identified at nucleotide positions 61 to 62, preferably wherein the assay is characterised as having a ROC AUC of at least 0.84.

In any of the assays described herein, the panel of one or more CpGs may comprise at least the 29,000 CpGs identified at nucleotide positions 61 to 62 in SEQ ID NOs 1 to 29,000, and further wherein the assay is characterised as having a ROC AUC of at least 0.84.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the described assays, the methylation status of the one or more CpGs in the panel is preferably determined by a β-value analysis, and the cancer is breast cancer or ovarian cancer. Preferably, the cancer is breast cancer.

In any of the assays described herein, the panel of one or more CpGs may comprise at least 500 CpGs selected from the CpGs identified at nucleotide positions 61 to 62 in SEQ ID NOs 1 to 29,000, optionally wherein:
1. the assay is characterised as having an ROC AUC (AUC) of at least 0.78, and more preferably wherein the panel of one or more CpGs comprises at least the CpGs identified in SEQ ID NOs 1 to 500 and identified at nucleotide positions 61 to 62;
2. the assay is characterised as having an AUC of at least 0.78, and more preferably wherein the panel of one or more CpGs comprises at least the CpGs identified in SEQ ID NOs 501 to 1000 and identified at nucleotide positions 61 to 62;
3. the assay is characterised as having an AUC of at least 0.77, and more preferably wherein the panel of one or more CpGs comprises at least the CpGs identified in SEQ ID NOs 1001 to 1500 and identified at nucleotide positions 61 to 62;
4. the assay is characterised as having an AUC of at least 0.74, and more preferably wherein the panel of one or more CpGs comprises at least the CpGs identified in SEQ ID NOs 1501 to 2000 and identified at nucleotide positions 61 to 62;
5. the assay is characterised as having an AUC of at least 0.77, and more preferably wherein the panel of one or more CpGs comprises at least the CpGs identified in SEQ ID NOs 2001 to 2500 and identified at nucleotide positions 61 to 62;
6. the assay is characterised as having an AUC of at least 0.72, and more preferably wherein the panel of one or more CpGs comprises at least the CpGs identified in SEQ ID NOs 2501 to 3000 and identified at nucleotide positions 61 to 62;
7. the assay is characterised as having an AUC of at least 0.75, and more preferably wherein the panel of one or more CpGs comprises at least the CpGs identified in SEQ ID NOs 3001 to 3500 and identified at nucleotide positions 61 to 62;
8. the assay is characterised as having an AUC of at least 0.70, and more CpGs identified in SEQ ID NOs 3501 to 4000 and identified at nucleotide positions 61 to 62;
9. the assay is characterised as having an AUC of at least 0.74, and more preferably wherein the panel of one or more CpGs comprises at least the CpGs identified in SEQ ID NOs 4001 to 4500 and identified at nucleotide positions 61 to 62;
10. the assay is characterised as having an AUC of at least 0.76, and more preferably wherein the panel of one or more CpGs comprises at least the CpGs identified in SEQ ID NOs 4501 to 5000 and identified at nucleotide positions 61 to 62;
11. the assay is characterised as having an AUC of at least 0.74, and more preferably wherein the panel of one or more CpGs comprises at least the CpGs identified in SEQ ID NOs 5001 to 5500 and identified at nucleotide positions 61 to 62;
12. the assay is characterised as having an AUC of at least 0.72, and more preferably wherein the panel of one or more CpGs comprises at least the CpGs identified in SEQ ID NOs 5501 to 6000 and identified at nucleotide positions 61 to 62;
13. the assay is characterised as having an AUC of at least 0.72, and more preferably wherein the panel of one or more CpGs comprises at least the CpGs identified in SEQ ID NOs 6001 to 6500 and identified at nucleotide positions 61 to 62;
14. the assay is characterised as having an AUC of at least 0.71, and more preferably wherein the panel of one or more CpGs comprises at least the CpGs identified in SEQ ID NOs 6501 to 7000 and identified at nucleotide positions 61 to 62;
15. the assay is characterised as having an AUC of at least 0.71, and more preferably wherein the panel of one or more CpGs comprises at least the CpGs identified in SEQ ID NOs 7001 to 7500 and identified at nucleotide positions 61 to 62;
16. the assay is characterised as having an AUC of at least 0.69, and more preferably wherein the panel of one or more CpGs comprises at least the CpGs identified in SEQ ID NOs 7501 to 8000 and identified at nucleotide positions 61 to 62;
17. the assay is characterised as having an AUC of at least 0.72, and more preferably wherein the panel of one or more CpGs comprises at least the CpGs identified in SEQ ID NOs 8001 to 8500 and identified at nucleotide positions 61 to 62;
18. the assay is characterised as having an AUC of at least 0.72, and more preferably wherein the panel of one or more CpGs comprises at least the CpGs identified in SEQ ID NOs 8501 to 9000 and identified at nucleotide positions 61 to 62;
19. the assay is characterised as having an AUC of at least 0.72, and more preferably wherein the panel of one or more CpGs comprises at least the CpGs identified in SEQ ID NOs 9001 to 9500 and identified at nucleotide positions 61 to 62;
20. the assay is characterised as having an AUC of at least 0.70, and more preferably wherein the panel of one or more CpGs comprises at least the CpGs identified in SEQ ID NOs 9501 to 10000 and identified at nucleotide positions 61 to 62;
21. the assay is characterised as having an AUC of at least 0.68, and more preferably wherein the panel of one or more CpGs comprises at least the CpGs identified in SEQ ID NOs 10001 to 10500 and identified at nucleotide positions 61 to 62;
22. the assay is characterised as having an AUC of at least 0.69, and more preferably wherein the panel of one or more CpGs comprises at least the CpGs identified in SEQ ID NOs 10501 to 11000 and identified at nucleotide positions 61 to 62;
23. the assay is characterised as having an AUC of at least 0.68, and more CpGs identified in SEQ ID NOs 11001 to 11500 and identified at nucleotide positions 61 to 62;
24. the assay is characterised as having an AUC of at least 0.64, and more preferably wherein the panel of one or more CpGs comprises at least the CpGs identified in SEQ ID NOs 11501 to 12000 and identified at nucleotide positions 61 to 62;
25. the assay is characterised as having an AUC of at least 0.67, and more preferably wherein the panel of one or more CpGs comprises at least the CpGs identified in SEQ ID NOs 12001 to 12500 and identified at nucleotide positions 61 to 62;
26. the assay is characterised as having an AUC of at least 0.68, and more preferably wherein the panel of one or more CpGs comprises at least the CpGs identified in SEQ ID NOs 12501 to 13000 and identified at nucleotide positions 61 to 62;
27. the assay is characterised as having an AUC of at least 0.69, and more preferably wherein the panel of one or more CpGs comprises at least the CpGs identified in SEQ ID NOs 13001 to 13500 and identified at nucleotide positions 61 to 62;
28. the assay is characterised as having an AUC of at least 0.67, and more preferably wherein the panel of one or more CpGs comprises at least the CpGs identified in SEQ ID NOs 13501 to 14000 and identified at nucleotide positions 61 to 62;
29. the assay is characterised as having an AUC of at least 0.66, and more preferably wherein the panel of one or more CpGs comprises at least the CpGs identified in SEQ ID NOs 14001 to 14500 and identified at nucleotide positions 61 to 62;
30. the assay is characterised as having an AUC of at least 0.63, and more preferably wherein the panel of one or more CpGs comprises at least the CpGs identified in SEQ ID NOs 14501 to 15000 and identified at nucleotide positions 61 to 62;
31. the assay is characterised as having an AUC of at least 0.69, and more preferably wherein the panel of one or more CpGs comprises at least the CpGs identified in SEQ ID NOs 15001 to 15500 and identified at nucleotide positions 61 to 62;
32. the assay is characterised as having an AUC of at least 0.64, and more preferably wherein the panel of one or more CpGs comprises at least the CpGs identified in SEQ ID NOs 15501 to 16000 and identified at nucleotide positions 61 to 62;
33. the assay is characterised as having an AUC of at least 0.64, and more preferably wherein the panel of one or more CpGs comprises at least the CpGs identified in SEQ ID NOs 16001 to 16500 and identified at nucleotide positions 61 to 62;
34. the assay is characterised as having an AUC of at least 0.66, and more preferably wherein the panel of one or more CpGs comprises at least the CpGs identified in SEQ ID NOs 16501 to 17000 and identified at nucleotide positions 61 to 62;
35. the assay is characterised as having an AUC of at least 0.61, and more preferably wherein the panel of one or more CpGs comprises at least the CpGs identified in SEQ ID NOs 17001 to 17500 and identified at nucleotide positions 61 to 62;
36. the assay is characterised as having an AUC of at least 0.64, and more preferably wherein the panel of one or more CpGs comprises at least the CpGs identified in SEQ ID NOs 17501 to 18000 and identified at nucleotide positions 61 to 62;
37. the assay is characterised as having an AUC of at least 0.61, and more preferably wherein the panel of one or more CpGs comprises at least the CpGs identified in SEQ ID NOs 18001 to 18500 and identified at nucleotide positions 61 to 62;
38. the assay is characterised as having an AUC of at least 0.62, and more CpGs identified in SEQ ID NOs 18501 to 19000 and identified at nucleotide positions 61 to 62;
39. the assay is characterised as having an AUC of at least 0.62, and more preferably wherein the panel of one or more CpGs comprises at least the CpGs identified in SEQ ID NOs 19001 to 19500 and identified at nucleotide positions 61 to 62;
40. the assay is characterised as having an AUC of at least 0.62, and more preferably wherein the panel of one or more CpGs comprises at least the CpGs identified in SEQ ID NOs 19501 to 20000 and identified at nucleotide positions 61 to 62;
41. the assay is characterised as having an AUC of at least 0.64, and more preferably wherein the panel of one or more CpGs comprises at least the CpGs identified in SEQ ID NOs 20001 to 20500 and identified at nucleotide positions 61 to 62;
42. the assay is characterised as having an AUC of at least 0.62, and more preferably wherein the panel of one or more CpGs comprises at least the CpGs identified in SEQ ID NOs 20501 to 21000 and identified at nucleotide positions 61 to 62;
43. the assay is characterised as having an AUC of at least 0.62, and more preferably wherein the panel of one or more CpGs comprises at least the CpGs identified in SEQ ID NOs 21001 to 21500 and identified at nucleotide positions 61 to 62;
44. the assay is characterised as having an AUC of at least 0.63, and more preferably wherein the panel of one or more CpGs comprises at least the CpGs identified in SEQ ID NOs 21501 to 22000 and identified at nucleotide positions 61 to 62;
45. the assay is characterised as having an AUC of at least 0.64, and more preferably wherein the panel of one or more CpGs comprises at least the CpGs identified in SEQ ID NOs 22001 to 22500 and identified at nucleotide positions 61 to 62;
46. the assay is characterised as having an AUC of at least 0.61, and more preferably wherein the panel of one or more CpGs comprises at least the CpGs identified in SEQ ID NOs 22501 to 23000 and identified at nucleotide positions 61 to 62;
47. the assay is characterised as having an AUC of at least 0.59, and more preferably wherein the panel of one or more CpGs comprises at least the CpGs identified in SEQ ID NOs 23001 to 23500 and identified at nucleotide positions 61 to 62;
48. the assay is characterised as having an AUC of at least 0.60, and more preferably wherein the panel of one or more CpGs comprises at least the CpGs identified in SEQ ID NOs 23501 to 24000 and identified at nucleotide positions 61 to 62;
49. the assay is characterised as having an AUC of at least 0.64, and more preferably wherein the panel of one or more CpGs comprises at least the CpGs identified in SEQ ID NOs 24001 to 24500 and identified at nucleotide positions 61 to 62;
50. the assay is characterised as having an AUC of at least 0.60, and more preferably wherein the panel of one or more CpGs comprises at least the CpGs identified in SEQ ID NOs 24501 to 25000 and identified at nucleotide positions 61 to 62;
51. the assay is characterised as having an AUC of at least 0.58, and more preferably wherein the panel of one or more CpGs comprises at least the CpGs identified in SEQ ID NOs 25001 to 25500 and identified at nucleotide positions 61 to 62;
52. the assay is characterised as having an AUC of at least 0.60, and more preferably wherein the panel of one or more CpGs comprises at least the CpGs identified in SEQ ID NOs 25501 to 26000 and identified at nucleotide positions 61 to 62;
53. the assay is characterised as having an AUC of at least 0.59, and more CpGs identified in SEQ ID NOs 26001 to 26500 and identified at nucleotide positions 61 to 62;
54. the assay is characterised as having an AUC of at least 0.61, and more preferably wherein the panel of one or more CpGs comprises at least the CpGs identified in SEQ ID NOs 26501 to 27000 and identified at nucleotide positions 61 to 62;
55. the assay is characterised as having an AUC of at least 0.56, and more preferably wherein the panel of one or more CpGs comprises at least the CpGs identified in SEQ ID NOs 27001 to 27500 and identified at nucleotide positions 61 to 62;
56. the assay is characterised as having an AUC of at least 0.62, and more preferably wherein the panel of one or more CpGs comprises at least the CpGs identified in SEQ ID NOs 27501 to 28000 and identified at nucleotide positions 61 to 62;
57. the assay is characterised as having an AUC of at least 0.60, and more preferably wherein the panel of one or more CpGs comprises at least the CpGs identified in SEQ ID NOs 28001 to 28500 and identified at nucleotide positions 61 to 62; or
58. the assay is characterised as having an AUC of at least 0.61, and more preferably wherein the panel of one or more CpGs comprises at least the CpGs identified in SEQ ID NOs 28501 to 29000 and identified at nucleotide positions 61 to 62.

In any of the described assays, the methylation status of the one or more CpGs in the panel is preferably determined by a β-value analysis, and the cancer is breast cancer or ovarian cancer. Preferably, the cancer is breast cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the assays described herein, the panel of one or more CpGs may comprise:
1. at least 100 CpGs selected from the CpGs identified at nucleotide positions 61 to 62 in SEQ ID NOs 1 to 29,000, preferably wherein the assay is characterised as having an AUC of at least 0.72, and more preferably wherein the panel of one or more CpGs comprises at least the CpGs identified in SEQ ID NOs 1 to 100 and identified at nucleotide positions 61 to 62;
2. at least 500 CpGs selected from the CpGs identified at nucleotide positions 61 to 62 in SEQ ID NOs 1 to 29,000, preferably wherein the assay is characterised as having an AUC of at least 0.78, and more preferably wherein the panel of one or more CpGs comprises at least the CpGs identified in SEQ ID NOs 1 to 500 and identified at nucleotide positions 61 to 62;
3. at least 1000 CpGs selected from the CpGs identified at nucleotide positions 61 to 62 in SEQ ID NOs 1 to 29,000, preferably wherein the assay is characterised as having an AUC of at least 0.81, and more preferably wherein the panel of one or more CpGs comprises at least the CpGs identified in SEQ ID NOs 1 to 1000 and identified at nucleotide positions 61 to 62;
4. at least 2000 CpGs selected from the CpGs identified at nucleotide positions 61 to 62 in SEQ ID NOs 1 to 29,000, preferably wherein the assay is characterised as having an AUC of at least 0.82, and more preferably wherein the panel of one or more CpGs comprises at least the CpGs identified in SEQ ID NOs 1 to 2000 and identified at nucleotide positions 61 to 62;
5. at least 3000 CpGs selected from the CpGs identified at nucleotide positions 61 to 62 in SEQ ID NOs 1 to 29,000, preferably wherein the assay is characterised as having an AUC of at least 0.83, and more preferably wherein the panel of one or more CpGs comprises at least the CpGs identified in SEQ ID NOs 1 to 3000 and identified at nucleotide positions 61 to 62;
6. at least 4000 CpGs selected from the CpGs identified at nucleotide positions 61 to 62 in SEQ ID NOs 1 to 29,000, preferably wherein the assay is characterised as having an AUC of at least 0.83, and more preferably wherein the panel of one or more CpGs comprises at least the CpGs identified in SEQ ID NOs 1 to 4000 and identified at nucleotide positions 61 to 62;
7. at least 5000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 5000 and identified at nucleotide positions 61 to 62;
8. at least 6000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 6000 and identified at nucleotide positions 61 to 62;
9. at least 7000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 7000 and identified at nucleotide positions 61 to 62;
10. at least 8000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 8000 and identified at nucleotide positions 61 to 62;
11. at least 9000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 9000 and identified at nucleotide positions 61 to 62;
12. at least 10000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 10000 and identified at nucleotide positions 61 to 62;
13. at least 11000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 11000 and identified at nucleotide positions 61 to 62;
14. at least 12000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 12000 and identified at nucleotide positions 61 to 62;
15. at least 13000 CpGs selected from the CpGs identified at nucleotide is characterised as having an AUC of at least 0.84, and more preferably wherein the panel of one or more CpGs comprises at least the CpGs identified in SEQ ID NOs 1 to 13000 and identified at nucleotide positions 61 to 62;
16. at least 14000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 14000 and identified at nucleotide positions 61 to 62;
17. at least 15000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 15000 and identified at nucleotide positions 61 to 62;
18. at least 16000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 16000 and identified at nucleotide positions 61 to 62;
19. at least 17000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 17000 and identified at nucleotide positions 61 to 62;
20. at least 18000 CpGs selected from the CpGs identified at nucleotide is characterised as having an AUC of at least 0.84, and more preferably wherein the panel of one or more CpGs comprises at least the CpGs identified in SEQ ID NOs 1 to 18000 and identified at nucleotide positions 61 to 62;
21. at least 19000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 19000 and identified at nucleotide positions 61 to 62;
22. at least 20000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 20000 and identified at nucleotide positions 61 to 62;
23. at least 21000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 21000 and identified at nucleotide positions 61 to 62;
24. at least 22000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 22000 and identified at nucleotide positions 61 to 62;
25. at least 23000 CpGs selected from the CpGs identified at nucleotide is characterised as having an AUC of at least 0.84, and more preferably wherein the panel of one or more CpGs comprises at least the CpGs identified in SEQ ID NOs 1 to 23000 and identified at nucleotide positions 61 to 62;
26. at least 24000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 24000 and identified at nucleotide positions 61 to 62;
27. at least 25000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 25000 and identified at nucleotide positions 61 to 62;
28. at least 26000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 26000 and identified at nucleotide positions 61 to 62;
29. at least 27000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 27000 and identified at nucleotide positions 61 to 62;
30. at least 28000 CpGs selected from the CpGs identified at nucleotide is characterised as having an AUC of at least 0.84, and more preferably wherein the panel of one or more CpGs comprises at least the CpGs identified in SEQ ID NOs 1 to 28000 and identified at nucleotide positions 61 to 62; or
31. at least 29000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 1 to 29000 and identified at nucleotide positions 61 to 62.

In any of the described assays, the methylation status of the one or more CpGs in the panel is preferably determined by a β-value analysis, and the cancer is breast cancer or ovarian cancer. Preferably, the cancer is breast cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the assays described herein, the panel of one or more CpGs may comprise:
1. at least 1000 CpGs selected from the CpGs identified at nucleotide positions 61 to 62 in SEQ ID NOs 1 to 29,000, preferably wherein the assay is characterised as having an AUC of at least 0.61, and more preferably wherein the panel of one or more CpGs comprises at least the CpGs identified in SEQ ID NOs 28,001 to 29,000 and identified at nucleotide positions 61 to 62;
2. at least 2000 CpGs selected from the CpGs identified at nucleotide positions 61 to 62 in SEQ ID NOs 1 to 29,000, preferably wherein the assay is characterised as having an AUC of at least 0.61, and more preferably wherein the panel of one or more CpGs comprises at least the CpGs identified in SEQ ID NOs 27,001 to 29,000 and identified at nucleotide positions 61 to 62;
3. at least 3000 CpGs selected from the CpGs identified at nucleotide positions 61 to 62 in SEQ ID NOs 1 to 29,000, preferably wherein the assay is characterised as having an AUC of at least 0.62, and more preferably wherein the panel of one or more CpGs comprises at least the CpGs identified in SEQ ID NOs 26,001 to 29,000 and identified at nucleotide positions 61 to 62;
4. at least 4000 CpGs selected from the CpGs identified at nucleotide positions 61 to 62 in SEQ ID NOs 1 to 29,000, preferably wherein the assay is characterised as having an AUC of at least 0.60, and more preferably wherein the panel of one or more CpGs comprises at least the CpGs identified in SEQ ID NOs 25,001 to 29,000 and identified at nucleotide positions 61 to 62;
5. at least 5000 CpGs selected from the CpGs identified at nucleotide positions 61 to 62 in SEQ ID NOs 1 to 29,000, preferably wherein the assay is characterised as having an AUC of at least 0.61, and more preferably wherein the panel of one or more CpGs comprises at least the CpGs identified in SEQ ID NOs 24,001 to 29,000 and identified at nucleotide positions 61 to 62;
6. at least 6000 CpGs selected from the CpGs identified at nucleotide positions 61 to 62 in SEQ ID NOs 1 to 29,000, preferably wherein the assay is characterised as having an AUC of at least 0.62, and more preferably wherein the panel of one or more CpGs comprises at least the CpGs identified in SEQ ID NOs 23,001 to 29,000 and identified at nucleotide positions 61 to 62;
7. at least 7000 CpGs selected from the CpGs identified at nucleotide positions 61 to 62 in SEQ ID NOs 1 to 29,000, preferably wherein the assay is characterised as having an AUC of at least 0.62, and more preferably wherein the panel of one or more CpGs comprises at least the CpGs identified in SEQ ID NOs 22,001 to 29,000 and identified at nucleotide positions 61 to 62;
8. at least 8000 CpGs selected from the CpGs identified at nucleotide positions 61 to 62 in SEQ ID NOs 1 to 29,000, preferably wherein the assay is characterised as having an AUC of at least 0.61, and more preferably wherein the panel of one or more CpGs comprises at least the CpGs identified in SEQ ID NOs 21,001 to 29,000 and identified at nucleotide positions 61 to 62;
9. at least 9000 CpGs selected from the CpGs identified at nucleotide positions 61 to 62 in SEQ ID NOs 1 to 29,000, preferably wherein the assay is characterised as having an AUC of at least 0.63, and more preferably wherein the panel of one or more CpGs comprises at least the CpGs identified in SEQ ID NOs 20,001 to 29,000 and identified at nucleotide positions 61 to 62;
10. at least 10000 CpGs selected from the CpGs identified at nucleotide positions 61 to 62 in SEQ ID NOs 1 to 29,000, preferably wherein the assay is characterised as having an AUC of at least 0.62, and more preferably wherein the panel of one or more CpGs comprises at least the CpGs identified in SEQ ID NOs 19,001 to 29,000 and identified at nucleotide positions 61 to 62;
11. at least 11000 CpGs selected from the CpGs identified at nucleotide positions 61 to 62 in SEQ ID NOs 1 to 29,000, preferably wherein the assay is characterised as having an AUC of at least 0.62, and more preferably wherein the panel of one or more CpGs comprises at least the CpGs identified in SEQ ID NOs 18,001 to 29,000 and identified at nucleotide positions 61 to 62;
12. at least 12000 CpGs selected from the CpGs identified at nucleotide is characterised as having an AUC of at least 0.62, and more preferably wherein the panel of one or more CpGs comprises at least the CpGs identified in SEQ ID NOs 17,001 to 29,000 and identified at nucleotide positions 61 to 62;
13. at least 13000 CpGs selected from the CpGs identified at nucleotide positions 61 to 62 in SEQ ID NOs 1 to 29,000, preferably wherein the assay is characterised as having an AUC of at least 0.63, and more preferably wherein the panel of one or more CpGs comprises at least the CpGs identified in SEQ ID NOs 16,001 to 29,000 and identified at nucleotide positions 61 to 62;
14. at least 14000 CpGs selected from the CpGs identified at nucleotide positions 61 to 62 in SEQ ID NOs 1 to 29,000, preferably wherein the assay is characterised as having an AUC of at least 0.63, and more preferably wherein the panel of one or more CpGs comprises at least the CpGs identified in SEQ ID NOs 15,001 to 29,000 and identified at nucleotide positions 61 to 62;
15. at least 15000 CpGs selected from the CpGs identified at nucleotide positions 61 to 62 in SEQ ID NOs 1 to 29,000, preferably wherein the assay is characterised as having an AUC of at least 0.63, and more preferably wherein the panel of one or more CpGs comprises at least the CpGs identified in SEQ ID NOs 14,001 to 29,000 and identified at nucleotide positions 61 to 62;
16. at least 16000 CpGs selected from the CpGs identified at nucleotide positions 61 to 62 in SEQ ID NOs 1 to 29,000, preferably wherein the assay is characterised as having an AUC of at least 0.64, and more preferably wherein the panel of one or more CpGs comprises at least the CpGs identified in SEQ ID NOs 13,001 to 29,000 and identified at nucleotide positions 61 to 62;
17. at least 17000 CpGs selected from the CpGs identified at nucleotide is characterised as having an AUC of at least 0.64, and more preferably wherein the panel of one or more CpGs comprises at least the CpGs identified in SEQ ID NOs 12,001 to 29,000 and identified at nucleotide positions 61 to 62;
18. at least 18000 CpGs selected from the CpGs identified at nucleotide positions 61 to 62 in SEQ ID NOs 1 to 29,000, preferably wherein the assay is characterised as having an AUC of at least 0.64, and more preferably wherein the panel of one or more CpGs comprises at least the CpGs identified in SEQ ID NOs 11,001 to 29,000 and identified at nucleotide positions 61 to 62;
19. at least 19000 CpGs selected from the CpGs identified at nucleotide positions 61 to 62 in SEQ ID NOs 1 to 29,000, preferably wherein the assay is characterised as having an AUC of at least 0.66, and more preferably wherein the panel of one or more CpGs comprises at least the CpGs identified in SEQ ID NOs 10,001 to 29,000 and identified at nucleotide positions 61 to 62;
20. at least 20000 CpGs selected from the CpGs identified at nucleotide positions 61 to 62 in SEQ ID NOs 1 to 29,000, preferably wherein the assay is characterised as having an AUC of at least 0.66, and more preferably wherein the panel of one or more CpGs comprises at least the CpGs identified in SEQ ID NOs 9,001 to 29,000 and identified at nucleotide positions 61 to 62;
21. at least 21000 CpGs selected from the CpGs identified at nucleotide positions 61 to 62 in SEQ ID NOs 1 to 29,000, preferably wherein the assay is characterised as having an AUC of at least 0.68, and more preferably wherein the panel of one or more CpGs comprises at least the CpGs identified in SEQ ID NOs 8,001 to 29,000 and identified at nucleotide positions 61 to 62;
22. at least 22000 CpGs selected from the CpGs identified at nucleotide is characterised as having an AUC of at least 0.66, and more preferably wherein the panel of one or more CpGs comprises at least the CpGs identified in SEQ ID NOs 7,001 to 29,000 and identified at nucleotide positions 61 to 62;
23. at least 23000 CpGs selected from the CpGs identified at nucleotide positions 61 to 62 in SEQ ID NOs 1 to 29,000, preferably wherein the assay is characterised as having an AUC of at least 0.69, and more preferably wherein the panel of one or more CpGs comprises at least the CpGs identified in SEQ ID NOs 6,001 to 29,000 and identified at nucleotide positions 61 to 62;
24. at least 24000 CpGs selected from the CpGs identified at nucleotide positions 61 to 62 in SEQ ID NOs 1 to 29,000, preferably wherein the assay is characterised as having an AUC of at least 0.71, and more preferably wherein the panel of one or more CpGs comprises at least the CpGs identified in SEQ ID NOs 5,001 to 29,000 and identified at nucleotide positions 61 to 62;
25. at least 25000 CpGs selected from the CpGs identified at nucleotide positions 61 to 62 in SEQ ID NOs 1 to 29,000, preferably wherein the assay is characterised as having an AUC of at least 0.72, and more preferably wherein the panel of one or more CpGs comprises at least the CpGs identified in SEQ ID NOs 4,001 to 29,000 and identified at nucleotide positions 61 to 62;
26. at least 26000 CpGs selected from the CpGs identified at nucleotide positions 61 to 62 in SEQ ID NOs 1 to 29,000, preferably wherein the assay is characterised as having an AUC of at least 0.75, and more preferably wherein the panel of one or more CpGs comprises at least the CpGs identified in SEQ ID NOs 3,001 to 29,000 and identified at nucleotide positions 61 to 62;
27. at least 27000 CpGs selected from the CpGs identified at nucleotide is characterised as having an AUC of at least 0.77, and more preferably wherein the panel of one or more CpGs comprises at least the CpGs identified in SEQ ID NOs 2,001 to 29,000 and identified at nucleotide positions 61 to 62;
28. at least 28000 CpGs selected from the CpGs identified at nucleotide positions 61 to 62 in SEQ ID NOs 1 to 29,000, preferably wherein the assay is characterised as having an AUC of at least 0.80, and more preferably wherein the panel of one or more CpGs comprises at least the CpGs identified in SEQ ID NOs 1,001 to 29,000 and identified at nucleotide positions 61 to 62;
29. at least 28500 CpGs selected from the CpGs identified at nucleotide positions 61 to 62 in SEQ ID NOs 1 to 29,000, preferably wherein the assay is characterised as having an AUC of at least 0.83, and more preferably wherein the panel of one or more CpGs comprises at least the CpGs identified in SEQ ID NOs 501 to 29,000 and identified at nucleotide positions 61 to 62;
30. at least 28900 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified in SEQ ID NOs 101 to 29,000 and identified at nucleotide positions 61 to 62; or
31. at least 29000 CpGs selected from the CpGs identified at nucleotide positions 61 to 62 in SEQ ID NOs 1 to 29,000, preferably wherein the assay is characterised as having an AUC of at least 0.84, and more preferably wherein the panel of one or more CpGs comprises at least the CpGs identified at nucleotide positions 61 to 62 in SEQ ID NOs 1 to 29,000.

In any of the described assays, the methylation status of the one or more CpGs in the panel is preferably determined by a β-value analysis, and the cancer is breast cancer or ovarian cancer. Preferably, the cancer is breast cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

The invention also provides a variety of assays, each comprising any 1, 2, 3, 4, 5, 6, 7, 8, 9, 10 or more (or any range derivable therein) of a variety of steps and in no particular order, including methods of the following: measuring in a sample; analyzing a sample; assessing a sample; evaluating a sample; measuring nucleic acids in a sample; assessing nucleic acids in a sample; detecting nucleic acids in a sample; measuring methylation in nucleic acids in a sample; analyzing nucleic acids in a sample; assessing nucleic acids in a sample; measuring methylation at one or more CpG dinucleotides in a sample; detecting methylation at one or more CpG dinucleotides in a sample; assaying methylation at one or more CpG dinucleotides in a sample; assessing methylation at one or more CpG dinucleotides in a sample; measuring a methylation status in a sample; assaying a methylation status in a sample; detecting methylation status in a sample; determining methylation status in a sample; identifying methylation status in a sample; measuring one or more DNA methylation markers in a sample; assessing one or more DNA methylation markers in a sample; detecting one or more DNA methylation markers in a sample; measuring the presence of methylation at one or more markers in a sample; detecting the presence of methylation at one or more markers in a sample; assessing the presence of methylation at one or more markers in a sample; assaying the presence of one of more markers in a sample; measuring one or more DNA methylation markers in a sample but excluding the measuring of one or more other DNA methylation markers in the sample; assessing one or more DNA methylation markers in a sample but excluding the assessing of one or more other DNA methylation markers in the sample; analyzing one or more DNA methylation markers in a sample but excluding the analyzing of one or more other DNA methylation markers in the sample; detecting one or more DNA methylation markers in a sample but excluding the detecting of one or more other DNA methylation markers in the sample; measuring methylation status in nucleic acids from a sample from tissue from an individual other than tissue from the individual suspected of, or at risk for, being cancerous; detecting methylation status in nucleic acids from a sample from tissue from an individual other than tissue from the individual suspected of, or at risk for, being cancerous; analyzing methylation status in nucleic acids from a sample from tissue from an individual other than tissue from the individual suspected of, or at risk for, being cancerous; assessing methylation status in nucleic acids from a sample from tissue from an individual other than tissue from the individual suspected of, or at risk for, being cancerous; measuring methylation at one or more CpG dinucleotides in a sample but excluding the measuring of methylation at one or more CpG dinucleotides in the sample; assessing methylation at one or more CpG dinucleotides in a sample but excluding the assessing of methylation at one or more CpG dinucleotides in the sample; analyzing methylation at one or more CpG dinucleotides in a sample but excluding the analyzing of methylation at one or more CpG dinucleotides in the sample; detecting methylation at one or more CpG dinucleotides in a sample but excluding the detecting of methylation at one or more CpG dinucleotides in the sample; measuring methylation at one or more CpG dinucleotides in nucleic acids from a sample from tissue from an individual other than tissue from the individual suspected of, or at risk for, being cancerous; detecting methylation at one or more CpG dinucleotides in nucleic acids from a sample from tissue from an individual other than tissue from the individual suspected of, or at risk for, being cancerous; analyzing methylation at one or more CpG dinucleotides in nucleic acids from a sample from tissue from an individual other than tissue from the individual suspected of, or at risk for, being cancerous; assessing methylation at one or more CpG dinucleotides in nucleic acids from a sample from tissue from an individual other than tissue from the individual suspected of, or at risk for, being cancerous; treating an individual for cancer when the individual has been determined to have a methylation status at one or more methylation markers; treating an individual for cancer when the individual has been determined to have methylation at one or more CpG dinucleotides;
wherein any of the aforementioned methods, or any other methods encompassed by the disclosure, may comprise any one or more of the following method steps:
measuring methylation status, wherein the measuring identifies the methylation status of one or markers from nucleic acids in a sample;
measuring methylation status, wherein the measuring identifies the presence of one or more markers from nucleic acids in a sample;
measuring the presence of one or more methylation markers from a sample;
providing DNA from a sample;
providing nucleic acids from a sample;
determining whether one or more methylation markers from nucleic acids from a sample are methylated;
measuring whether one or more methylation markers from nucleic acids from a sample are methylated;
performing a sequencing step on nucleic acids from a sample;
determining a sequence of nucleic acids from a sample;
performing bisulphite conversion on one or more markers;
performing bisulphite conversion on one or more CpG dinucleotides;
hybridizing DNA to an array comprising probes capable of determining methylated versus non-methylated markers;
hybridizing DNA to an array comprising probes capable of determining methylated versus non-methylated CpG dinucleotides;
hybridizing DNA to an array comprising probes capable of discriminating between methylated and non-methylated markers;
hybridizing DNA to an array comprising probes capable of discriminating between methylated and non-methylated CpG dinucleotides;
performing an amplification step on sequence from nucleic acids from a sample;
performing an amplification step on sequence from nucleic acids using methylation-specific primers;
performing amplification of sequence comprising one or more regions suspected of having methylation or in need of determination of a methylation status;
performing PCR on sequence comprising one or more regions suspected of having methylation or in need of determination of a methylation status;
performing a capturing step;
performing a binding step;
performing a purification step;
performing a capturing step comprising binding of polynucleotides comprising one or more methylation markers to binding molecules specific to the one or more methylation markers and collecting complexes thereof;
stratifying the grade of a cancer;
determining the risk of cancer;
determining the risk of recurrence of cancer;
obtaining a sample from an individual;
obtaining DNA from a sample from an individual;
administering a treatment to an individual;
providing DNA from a sample;
determining whether one or more methylation markers from a panel of methylation markers comprises a specific sequence; and/or
obtaining data that identifies whether each one of a group of methylation markers from a panel comprises a specific sequence.

Moreover, in some aspects of the invention, an individual who is administered a therapy or treatment has been subjected to any of the methods and steps described herein.

Described herein are assays that utilise a statistically robust panel of one or more CpGs whose methylation status can be determined to provide a reliable prediction of the presence or development of cancer in an individual. By determining the methylation status of each CpG within the panel of one or more CpGs, a cancer index value may be derived thus enabling stratification of individuals according to their risk of developing cancer or of having cancer, particularly breast and/or ovarian cancer, with statistically robust sensitivity and specificity. The skilled person would understand that the methylation status of each CpG within a panel of one or more CpGs can be determined by any suitable means in order to thereby derive the cancer index value. Any one method, or a combination of methods, may be used to determine the methylation status of each CpG within a panel of one or more CpGs.

Various exemplary methods for determining the methylation status of each CpG within a panel of one or more CpGs are described herein. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

### Cancer Index Values as Diagnostic and Risk Assessment Tools

In any of the assays described herein, the assessment of the presence, absence or development of cancer in an individual is based on the cancer index value of the individual at the time of testing.

As explained herein, using panels of the specific CpGs disclosed herein, cancer index values can be established which correspond with cancer negative samples, because they are based on values derived from individuals known to be cancer negative and obtained from samples from an anatomical site other than the breast or ovary, such as the cervix, the vagina, the buccal area, blood and/or urine, preferably a cervical liquid-based cytology sample and most preferably a cervical smear sample. Similarly, using panels of the specific CpGs disclosed herein, cancer index values can be established which correspond with cancer positive samples, because they are based on values derived from samples from an anatomical site other than the breast or ovary, as noted above, from tissue samples from individuals known to be cancer positive. A user can then apply these cancer index values to assess the presence, absence or development of cancer in any test individual whose cancer status is required to be tested. As also explained herein, the assays of the invention are capable of being performed with a high degree of statistical accuracy.

As explained herein, the described assays particularly relate to the assessment of the presence, absence or development of breast cancer and/or ovarian cancer.

A skilled person will readily appreciate that a cancer index value provides a value that indicates a “likelihood” or “risk” or “prediction” of any of the assays of the invention correctly assessing the presence, absence or development of cancer in an individual. This is because the assessment is based upon a correlation between DNA methylation profiles of tissue samples and individual disease status. Nevertheless, as demonstrated by data set out in the Examples and elsewhere herein, the assays of the invention provide such correlations with high statistical accuracy, thus providing the skilled person with a high degree of confidence that the cancer index value which is determined for any test individual whose cancer status is required to be tested will provide an accurate correlation with actual disease status for the individual.

In the context of the present invention, “likelihood”, “risk” and “prediction” may be used synonymously with each other.

Any references herein to sequences, genomic sequences and/or genomic coordinates are derived based upon Homo sapiens (human) genome assembly GRCh37 (hg19). The skilled person would understand variations in the nucleotide sequences of any given sequence, may exist due to sequencing errors and/or variation between individuals.

The assay of the invention represents a ‘prediction’ because any cancer index value (WID-BC-Index) derived in accordance with the invention is unlikely to be capable of diagnosing every individual as having or not having cancer with 100% specificity and 100% sensitivity. Rather, depending on the cancer index cutpoint threshold applied by the user for positively predicting the presence of cancer in an individual, the false positive and false negative rate will vary. In other words, the inventors have discovered that the assays of the invention can achieve variable levels of sensitivity and specificity for predicting the presence, absence or development of cancer, as defined by receiver operating characteristics, depending on the cancer index cutpoint threshold chosen and applied by the user. Such sensitivity and specificity can be seen from the data disclosed herein to be achievable at high proportions, demonstrating accurate and statistically-significant discriminatory capability.

Similarly, cancer index values which have been pre-determined to correlate with specific cancer phenotypes, such as the presence or absence of cancer, have been defined with a high level of statistical accuracy as explained further herein.

Assessing the ‘development’ of cancer in the context of the invention may refer to assessing whether an individual is likely or unlikely to develop cancer. The inventors have shown that the CpGs assayed in order to derive the cancer index value of the assays of the invention are representative of the cells within normal tissue from an anatomical site other than the breast or ovary, such as the cervix, the vagina, the buccal area, blood and/or urine, preferably a cervical liquid-based cytology sample and most preferably a cervical smear sample. As such, cells from these tissues canact as a surrogate for breast and/or ovarian cells that may transform to cancer. In more detail, the cancer index value derived in accordance with the present invention has been show to progressively increase in samples from a normal cervix, vagina, buccal area, blood and/or urine preferably cervical liquid-based cytology and most preferably derived from a cervical smear sample, in healthy women, to samples from corresponding tissue in women with a BRCA1 and/or BRCA2 germline mutation. The cancer index value derived in accordance with the invention has been show to further progressively increase in normal breast tissue surrounding a cancer lesion, and increase further in the cancer lesion itself, and increase yet further in a triple negative breast cancer lesion. This indicates that the breast cancer index value, determined in accordance with the present invention, is capable of identifying individuals that may be at risk of developing cancer, and are thereby pre-disposed to breast cancer or ovarian cancer, preferably breast cancer. Thus, assessing the development of cancer in accordance with the assays of the invention may refer to assessing an increased or decreased likelihood of cancer development, particularly breast cancer and ovarian cancer, preferably breast cancer. Assessing of the development of cancer in accordance with the assays of the invention may refer to assessing progression or worsening of a pre-existing cancer lesion in an individual. Assessment of the development of cancer in accordance with the assays of the invention may refer to predicting the likelihood of recurrence of cancer.

In any of the assays described herein, the step of assessing the presence or development of cancer in an individual based on a cancer index value may involve the application of a threshold value. Threshold values can provide a risk-based indication of an individual's cancer status, whether that is cancer positive, or cancer negative. Threshold values can also provide a means for identifying whether the cancer index value is intermediate between a cancer positive value and a cancer negative value. As explained herein, the cancer index value may be dynamic and subject to change depending upon genetic and/or environmental factors. Accordingly, the cancer index value may provide a means for assessing and monitoring cancer development. Cancer index values may therefore indicate at least a low risk or a high risk that the individual has a cancer positive status or has a status that is indicative of the development of cancer. If the cancer index value of an individual is determined by the assays of the invention at two or more time points, an increase or decrease in the individual's cancer index value may indicate an increased or decreased risk of the individual having or developing cancer, particularly breast and/or ovarian cancer, preferably breast cancer.

Throughout the disclosure herein the terms “threshold value”, “cutpoint”, and “cutpoint threshold” are to be considered synonymous and interchangeable.

As explained further herein any assay of the invention is an assay for assessing the presence, absence or development of cancer in an individual. The types of cancer are set out further herein. As explained further herein, the assays of the invention provide means for assessing whether an individual is at risk of having or developing cancer based on specific cutpoint thresholds. Such risk assessments can be provided with a high degree of confidence based on the statistical parameters which characterise the assay. Thus in any of the assays described herein involving cancer index cutpoint thresholds, the cutpoint threshold may be used for risk assessment purposes. Equally, in any of the assays described herein involving cancer index cutpoint thresholds, the cutpoint threshold value may be used to specify whether or not an individual has cancer as a pure diagnostic test. Again, such diagnostic tests can be provided with a high degree of confidence based on the statistical parameters which characterise the assay. Accordingly, in any assay described herein which specifies that a cancer index value for the individual is a specific value or more, or is “about” a specific value or more, the individual may be assessed as having cancer. In any assay described herein which specifies that a cancer index value for the individual is less than a specific value, or is less than “about” a specific value, the individual may be assessed as not having cancer. The term “about” is to be understood as providing a range of +/−5% of the value.

Accordingly, any assay of the invention is an assay for assessing the presence, absence or development of cancer in an individual, the assay comprising:
e. providing a sample which has been taken from the individual, the sample comprising a population of DNA molecules;
f. determining in the population of DNA molecules in the sample the methylation status of a panel of one or more CpGs selected from a panel of CpGs identified at nucleotide positions 61 to 62 in SEQ ID NOs 1 to 29,000;
g. deriving a cancer index value based on the methylation status of the one or more CpGs in the panel; and
h. assessing the presence, absence or development of cancer in the individual based on the cancer index value;

wherein the assay is characterised as having an area under the curve (AUC) of 0.60 or more as determined by receiver operating characteristics (ROC).

Such an assay may be performed in accordance with any of the methods disclosed and defined herein.

As explained further herein, any assay of the invention for assessing the presence, absence or development of cancer in an individual may alternatively be referred to as an assay for stratifying an individual in accordance with their cancer status.

Accordingly, any assay of the invention is an assay for stratifying an individual for the presence, absence or development of cancer in an individual, the assay comprising:
a. providing a sample which has been taken from the individual, the sample comprising a population of DNA molecules;
b. determining in the population of DNA molecules in the sample the methylation status of a panel of one or more CpGs selected from a panel of CpGs identified at nucleotide positions 61 to 62 in SEQ ID NOs 1 to 29,000;
c. deriving a cancer index value based on the methylation status of the one or more CpGs in the panel; and
d. stratifying the individual for the presence, absence or development of cancer based on the cancer index value;

wherein the assay is characterised as having an area under the curve (AUC) of 0.60 or more as determined by receiver operating characteristics (ROC).

Such an assay may be performed in accordance with any of the methods disclosed and defined herein.

Accordingly, any assay of the invention is an assay for stratifying an individual for cancer, the assay comprising:
a. providing a sample which has been taken from the individual, the sample comprising a population of DNA molecules;
b. determining in the population of DNA molecules in the sample the methylation status of a panel of one or more CpGs selected from a panel of CpGs identified at nucleotide positions 61 to 62 in SEQ ID NOs 1 to 29,000;
c. deriving a cancer index value based on the methylation status of the one or more CpGs in the panel; and
d. stratifying the individual for cancer based on the cancer index value;

wherein the assay is characterised as having an area under the curve (AUC) of 0.60 or more as determined by receiver operating characteristics (ROC).

Such an assay may be performed in accordance with any of the methods disclosed and defined herein.

The cancer index value may be derived by any suitable means. Preferably, the cancer index value may be derived by assessing the methylation status of the one or more CpGs in the panel selected from a panel of CpGs identified at nucleotide positions 61 to 62 in SEQ ID NOs 1 to 29,000, in a sample provided from an individual. The methylation status of the CpGs may be determined by any suitable means. For example, in any of the assays described herein the step of determining the methylation status of each CpG in the panel of one or more CpGs may comprise:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

Preferably, the the step of determining in the population of DNA molecules in the sample the methylation status of a panel of one or more CpGs further comprises determining a (3 value of each CpG. Deriving the cancer index value may involve providing a methylation β-value data set comprising the methylation β-values for each CpG in the panel of one or more CpGs. Optionally deriving the cancer index value may also involve estimating the fraction of contaminating DNA (e.g. immune cell DNA) within the DNA provided from a sample.

DNA may be DNA originating from a particular source organism, tissue or cell type. Preferably the contaminating DNA originates from one or more different cell types to one or more cell types of interest. A cell type of interest may particularly be an epithelial cell. In some aspects of the invention, it may be preferable to estimate the fraction of contaminating DNA after the step of providing a sample which has been take from an individual. The assays described herein may optionally involve estimating a contaminating DNA fraction within DNA in the sample by any suitable means. Preferably, the contaminating DNA fraction for the sample is estimated via any suitable bioinformatics analysis tool. A bioinformatics analysis tool that may be used to estimate a contaminating DNA fraction may be EpIDISH. As described herein, it may be desirable to estimate the fraction of contaminating DNA from the one or more cell types that are different to the one or more cell types of interest because the cancer index value used for predicting the presence or development of cancer in an individual may, in some instances, only be reliably derived from determining the methylation status of a set of CpGs from DNA of a particular cell type of interest. Particularly, methylation status beta-values may differ in the one or more cell types of interest within a sample relative to methylation status beta-values in contaminating DNA from different cell types within the same sample. Thus, the derived cancer index value may in some instances have a decreased predictive power without estimating and controlling for the contaminating DNA fraction within the DNA provided from the sample. In assays of the invention that involve estimating the fraction of contaminating DNA and accordingly controlling for said contaminating DNA, it is preferable to estimate an immune cell DNA fraction within the DNA provided from the sample. In particular assays of the invention, wherein the individual has an immune cell contamination of over 50% (i.e. wherein more than 50% of the DNA in the sample is deemed to be derived from immune cells), the assay may preferably involve controlling for the immune cell contamination by deriving the cancer index, in accordance with the invention, solely from the DNA molecules derived from epithelial cells.

Any of the assays described herein comprising a step of deriving a cancer index value based on the methylation status of the one or more CpGs in the panel may further comprise applying an algorithm to the methylation beta-value dataset to obtain the cancer index value. Preferably, in any of the assays described herein, the step of deriving the cancer index value based on the methylation status of the panel of CpGs comprises providing a methylation beta-value data set comprising the methylation beta-values for each CpG in the panel and applying an algorithm to the methylation beta-value data set to obtain the cancer index value.

In any of the assays described herein, the step of deriving the cancer index value based on the methylation status of the one or more CpGs in the panel comprises:
a. providing a methylation β-value data set comprising the methylation β-values for each CpG in the panel;
b. providing a mathematical model capable of generating the cancer index from the methylation β-value data set; and
c. applying the mathematical model to the methylation β-value data set, thereby generating the cancer index.

In any of the assays described herein, the cancer index value may be calculated by any suitable mathematical model such as an algorithm or formula. Preferably, the cancer index value is termed Women's risk Identification for Breast Cancer Index (WID-BC-index) and wherein the mathematical model which is applied to the methylation β-value data set to generate the cancer index is calculated by an algorithm according to the following formula:

$$WID - BC - index = {\sum }_{i = 1}^{n} \frac{μ - {w}_{i} {β}_{i}}{σ}$$

wherein:
a. β₁, . . . , βₙ are methylation beta-values (between 0 and 1);
b. w₁, . . . , w_{29,000} are real valued coefficients;
c. μ and σ are real valued parameters used to scale the index; and
d. n refers to the number of CpGs in the set of test CpGs;

preferably wherein the cancer is ovarian cancer.

In any of the assays described herein, the WID-BC-index algorithm applies real value coefficients inferred by initially training on a dataset (this dataset in the exemplary embodiments of the invention described in the Examples consisted of 217 breast cancer cases and 572 controls) to fit a ridge classifier using the R package glmnet with a mixing parameter value of alpha=0 (ridge penalty) and binomial response type. Ten-fold cross-validation was used internally by the cv.glmnet function in order to determine the optimal value of the regularisation parameter lambda. The beta values from n CpGs for individual ν, β₁^{ν}, . . . , βₙ^{ν}, are used as inputs to the ridge classifier. The coefficients w₁, . . . , wₙ are obtained from the fitted model. The following quantity was computed for each individual v in the training set:

$${x}_{v} = {\sum }_{i = 1}^{n} {w}_{i} {β}_{i}^{v}$$

Any suitable real valued coefficients may be applied to the WID-BC-Index in any of the assays described herein.

The value of the parameters μ and σ are given by the mean and standard deviation of x_{ν} in the training dataset respectively.

Thus, any suitable μ and σ real valued parameters may be applied to the WID-BC-index in any of the assays described herein. Any suitable training data set may be applied to the assays described herein in order to infer real value parameters and coefficients that can subsequently be applied to the WID-BC-index formula according to the present invention. Exemplary ways of utilising a training dataset in accordance with the present invention are further described in the ‘Statistical Analyses for Classifier Development’ section of the Materials and Methods section of the Examples.

Exemplary μ and σ real valued parameters are provided in Table 1 for CpG subsets identified in SEQ ID NOs 1 to 29,000. These real valued parameters may be applied to any of the assays described herein wherein the real parameters correspond to any one of the sets of CpGs identified in SEQ ID NOs 1 to 29,000 and set out in the left hand column of Table 1.

TABLE 1
Exemplary μ and σ real valued parameters are provided in
Table 1 for CpG subsets identified in SEQ ID NOs 1 to 29,000

| | mu | sigma |
|---|---|---|
| 1-100 | 1.802459586 | 1.656270817 |
| 1-500 | 2.616861104 | 2.85547826 |
| 1-1000 | 0.706240996 | 3.76930138 |
| 1-2000 | −2.751311174 | 4.334044068 |
| 1-3000 | −3.389185274 | 4.476544913 |
| 1-4000 | −5.032110623 | 4.623163512 |
| 1-5000 | −6.09198663 | 4.757014538 |
| 1-6000 | −6.334609741 | 4.801754622 |
| 1-7000 | −6.464186221 | 4.829523686 |
| 1-8000 | −6.182002383 | 4.851226293 |
| 1-9000 | −6.284348293 | 4.789306299 |
| 1-10000 | −6.328704187 | 4.960804433 |
| 1-11000 | −5.919447725 | 4.971132834 |
| 1-12000 | −5.312253693 | 4.979966579 |
| 1-13000 | −5.295876334 | 4.984380015 |
| 1-14000 | −5.593824433 | 4.991011031 |
| 1-15000 | −5.754919225 | 4.995382643 |
| 1-16000 | −5.615422764 | 4.99765114 |
| 1-17000 | −5.516580287 | 5.000467792 |
| 1-18000 | −5.54155042 | 5.002885671 |
| 1-19000 | −5.473472369 | 4.925317756 |
| 1-20000 | −5.650564107 | 5.006168191 |
| 1-21000 | −5.547857121 | 5.007066548 |
| 1-22000 | −5.478733023 | 5.007952537 |
| 1-23000 | −5.355319817 | 4.84946768 |
| 1-24000 | −5.518865053 | 5.008154299 |
| 1-25000 | −5.50792209 | 5.008628153 |
| 1-26000 | −5.445141511 | 5.008685384 |
| 1-27000 | −5.384804459 | 4.92985421 |
| 1-28000 | −5.440152727 | 5.008757086 |
| 1-29000 | −5.373148368 | 4.851176995 |

Exemplary w₁, . . . , wₙ real value coefficients are provided below for the CpGs identified at positions 61 to 62 in SEQ ID NOs 1 to 29,000. These real value coefficients may be applied to any of the assays described herein wherein the real parameters correspond to any one of the sets of CpGs identified in SEQ ID NOs 1 to 29,000 wherein the 29,000 real value coefficients below in turn correspond to the CpGs in turn identified at nucleotide positions 61 to 62 in SEQ ID NOs 1 to 29,000. Accordingly, the listed coefficients are presented below in numerical order corresponding respectively to the CpGs identified in SEQ ID NOs 1 to 5000. Thus the first number below corresponds to SEQ ID NO 1, the second number corresponds to SEQ ID NO 2 etc. The exemplary real value coefficients are as follows:

−0.87359, −0.5494, 0.54381, −0.50414, 0.49734, 0.49357, −0.49285, −0.48972, −0.48553, −0.48535, −0.48194, 0.47108, 0.46888, −0.46692, −0.46373, 0.44945, 0.44607, 0.44483, −0.4445, −0.43581, 0.43427, −0.42868, −0.42193, −0.41962, −0.41856, 0.4157, −0.41567, −0.41257, 0.4117, −0.4116, 0.40783, 0.40778, −0.40755, −0.40554, −0.40288, −0.40278, 0.39996, −0.39728, 0.39603, −0.39297, −0.38925, 0.38822, −0.38364, 0.38178, 0.37925, −0.378, −0.37623, 0.37542, −0.3744, 0.37397, −0.37305, 0.37273, 0.37068, 0.37045, 0.37009, −0.36972, −0.36961, −0.36945, −0.36724, −0.36398, 0.36311, −0.36082, 0.3598, 20−0.35824, −0.35811, −0.35792, 0.35687, −0.35512, −0.35408, 0.3535, 0.35228, −0.3505, −0.34865, 0.34717, 0.34699, −0.34648, 0.34503, 0.34366, −0.34357, 0.34304, −0.34251, 0.3424, −0.34179, 0.34102, −0.33907, −0.33761, 0.33675, 0.33637, −0.33472, 0.33471, −0.33468, 0.33438, −0.33238, −0.33238, 0.33026, 0.32991, 0.32796, −0.32787, −0.32629, 0.32606, −0.32586, 0.32488, 0.32471, 0.32266, −0.32183, 0.32012, 0.3191, 0.31814, −0.31771, −0.31753, −0.31702, 0.31531, −0.31478, 0.31455, 0.31367, 0.31353, 0.31249, −0.3123, 0.31173, 0.31107, 0.31086, −0.31064, −0.3105, 0.30944, 0.30912, 0.3087, 0.30839, 0.30811, −0.30786, 0.30778, 0.30776, 0.3074, −0.30639, 0.30501, −0.30328, −0.3024, 0.30206, 0.3013, 0.30072, −0.30012, 0.29941, 0.29912, −0.29887, −0.29879, −0.29754, −0.29647, −0.29596, −0.29591, 0.29463, −0.29439, −0.29397, 0.29336, 0.29205, −0.292, 0.29188, 0.29107, −0.29086, −0.29073, −0.29038, 0.28998, 0.28856, 0.28828, −0.28747, 0.2872, 0.2871, 0.2866, −0.28644, 0.28575, −0.2851, 0.28403, −0.28358, −0.28296, −0.28251, 0.28237, 0.28168, 0.28139, 0.28066, −0.28024, 0.27997, −0.2797, 0.27967, −0.2792, −0.27914, −0.27909, −0.27868, −0.2785, 0.27809, −0.27802, 0.27793, 0.27737, −0.27727, −0.27702, 0.27655, 0.27643, −0.27638, 0.27619, 0.27551, 0.27488, 0.27453, −0.27421, −0.27417, −0.27376, 0.27361, 0.2736, 0.27353, −0.27343, −0.27254, 0.27171, 0.2717, −0.27114, −0.27107, −0.27034, −0.27017, 0.27014, −0.26948, 0.26943, −0.269, −0.26841, −0.26786, 0.26761, 0.26744, −0.267, −0.26697, −0.26642, −0.26615, −0.26608, 0.26563, 0.26557, 0.26505, 0.26499, 0.26497, −0.26496, −0.26462, 0.26447, 0.26437, −0.26409, −0.26327, −0.26177, −0.26173, −0.26083, 0.2608, 0.26074, −0.26072, −0.26014, 0.2597, −0.25905, −0.25884, −0.25865, 0.25851, 0.2585, −0.25787, −0.25671, −0.25653, −0.2564, −0.25577, −0.25526, −0.25525, 0.25524, −0.25485, 0.25461, 0.25438, −0.25385, 0.25365, 0.25343, −0.25328, −0.25328, −0.25323, −0.25311, 0.25299, 0.25278, 0.25267, 0.25242, −0.25236, −0.25234, −0.25209, 0.25192, 0.25182, 0.25177, 0.25162, −0.25162, −0.25159, 0.25153, 0.25132, 0.25099, 0.25073, −0.25069, 0.25036, 0.24998, 0.2493, 0.24908, −0.24889, 0.24834, −0.24832, 0.24832, −0.24798, 0.24781, 0.24737, 0.24696, −0.24642, 0.24612, 0.24609, −0.24604, 0.24598, 0.24594, 0.24558, 0.24545, 0.24542, 0.24449, 0.24448, 0.24358, 0.24319, −0.24314, 0.24304, 0.24303, 0.24295, 0.24293, 0.24278, −0.24273, 0.24245, 0.24238, 0.24201, 0.24176, 0.24168, −0.24164, 0.24153, −0.2415, 0.2409, 0.24067, −0.24013, −0.24012, −0.23969, −0.23938, −0.23882, −0.23852, 0.23847, 0.23834, −0.23825, −0.23818, 0.23814, −0.23786, 0.23785, 0.23783, −0.23711, 0.23627, −0.23623, −0.23593, 0.23573, 0.23553, 0.23537, 0.23518, −0.23485, −0.23478, −0.23467, 0.23428, −0.23424, 0.23402, −0.23399, 0.2337, −0.23338, −0.23334, 0.23304, −0.23299, 0.23289, −0.23259, 0.23241, 0.23237, 0.23215, 0.23209, −0.23194, −0.23084, 0.23022, 0.22982, 0.22933, 0.22917, 0.22904, 0.22867, 0.22836, −0.22826, −0.22823, 0.22821, −0.22818, 0.22806, 0.22796, 0.22759, −0.22742, 0.2274, 0.22691, −0.22661, −0.2266, −0.22658, 0.22656, 0.22641, −0.22636, −0.22635, −0.22617, 0.2261, −0.22609, −0.22603, 0.22583, 0.2258, 0.2258, 0.22559, −0.22509, −0.22494, 0.22465, −0.22437, −0.22429, 0.22403, 0.22366, −0.22355, 0.2233, 0.22326, −0.22325, −0.22313, −0.22309, −0.22308, 0.22301, 0.22293, 0.22252, −0.22249, 0.22187, −0.2218, −0.22166, −0.22135, 0.22123, 0.22117, −0.22105, −0.221, −0.22096, 0.22095, −0.22089, −0.2207, −0.22067, −0.22059, 0.2205, 0.22004, −0.21995, −0.21939, −0.2192, 0.21901, −0.21884, −0.21876, 0.21868, −0.21845, 0.21821, 0.21818, 0.21791, −0.2177, −0.21745, 0.2173, −0.21726, −0.21692, −0.21679, −0.21674, 0.21669, −0.21667, −0.21666, 0.21648, −0.21644, −0.21641, 0.21641, 0.21604, −0.21602, −0.21602, 0.21573, 0.2155, 0.21532, −0.21531, −0.21528, −0.21504, −0.21499, −0.21465, −0.21458, 0.21427, −0.21417, 0.21385, −0.21296, −0.21259, 0.21206, −0.21192, 0.2118, 0.21161, −0.21147, −0.21124, −0.21113, −0.21113, −0.21079, 0.20989, 0.20979, 0.20965, 0.20954, 0.20917, −0.20893, 0.20887, −0.20874, 0.20873, −0.20862, −0.20854, 0.20836, 0.20828, 0.20826, 0.20824, −0.20821, 0.20816, −0.20803, 0.20801, 0.20767, 0.20751, 0.20747, −0.20736, −0.20721, −0.20717, −0.20702, −0.20701, −0.20681, −0.20678, 0.2067, −0.20657, 0.20657, 0.20651, −0.20611, 0.20608, 0.20592, 0.20589, 0.20569, −0.20557, 0.20509, −0.20509, 0.20495, −0.20494, −0.20491, 0.20472, 0.20452, −0.20423, 0.20399, −0.20393, −0.20376, −0.20374, 0.20355, −0.20351, 0.20342, 0.20325, −0.20322, −0.20318, 0.20318, 0.20298, −0.20281, −0.2028, −0.20258, 0.20242, 0.20221, 0.20212, 0.20204, 0.20196, 0.2018, −0.20157, 0.20065, −0.20063, −0.20059, −0.20053, −0.20053, −0.2002, 0.19999, −0.19973, −0.19965, 0.19952, −0.19914, −0.19905, −0.19864, 0.1985, −0.19845, −0.19816, 0.19784, 0.19772, 0.19771, 0.19769, 0.19722, 0.19704, 0.19702, −0.19688, 0.19665, 0.19661, 0.19655, 0.19649, −0.19647, −0.19626, −0.19611, −0.19534, −0.19533, 0.19524, 0.19522, 0.19451, 0.19425, −0.19412, −0.19406, −0.19401, 0.19388, 0.19371, 0.19365, −0.19361, −0.19349, 0.19311, 0.19304, 0.19288, −0.19275, 0.19268, 0.19262, 0.19249, −0.19209, 0.19208, −0.19182, 0.19162, 0.19158, −0.19154, 0.19153, 0.19125, −0.19117, −0.19079, −0.19038, 0.19022, −0.1901, −0.19007, −0.19005, −0.19004, −0.18981, −0.1898, 0.18962, −0.18932, 0.18931, −0.1892, −0.18918, 0.18898, −0.18887, −0.18881, 0.18878, −0.18861, 0.18834, 0.1882, −0.18819, 0.18795, 0.18783, −0.1878, −0.18753, 0.18737, −0.18708, 0.18694, −0.18672, 0.18672, 0.18663, −0.18655, 0.18643, 0.18638, 0.18637, −0.18626, 0.1862, 0.1861, −0.1861, −0.18609, 0.18603, 0.18577, −0.18541, −0.1852, 0.18516, −0.18511, 0.18493, 0.18472, −0.18465, −0.18441, 0.18426, 0.18422, 0.18405, −0.18403, −0.18386, −0.18367, 0.18359, −0.18355, −0.18343, −0.18342, −0.18336, −0.18283, 0.18278, 0.18256, 0.1825, 0.1824, −0.18236, −0.18235, 0.18233, 0.18232, 0.18209, −0.18208, 0.18203, 0.18198, 0.18194, −0.18187, −0.18172, 0.18161, −0.1816, −0.18153, 0.18125, 0.18121, 0.18119, 0.18118, −0.18102, −0.18095, −0.18078, −0.18067, 0.18057, 0.18056, 0.18055, −0.18049, 0.1804, 0.18013, −0.17996, −0.17991, −0.17991, 0.1799, −0.17988, 0.17975, 0.17971, −0.17959, −0.17953, 0.17929, −0.17925, 0.17919, −0.17919, 0.17908, −0.17903, 0.17889, 0.17887, 0.17872, −0.17868, −0.17866, 0.17865, −0.17823, 0.17819, 0.17811, −0.17806, 0.17803, 0.17799, 0.1779, 0.17755, −0.17747, 0.17709, −0.17708, 0.17691, −0.17663, 0.1764, −0.17635, −0.17613, −0.17609, −0.1759, −0.17584, 0.17561, 0.17552, −0.17543, −0.17537, 0.17534, −0.17511, −0.17504, 0.17492, 0.17468, 0.17465, 0.17461, 0.17448, 0.17419, 0.17415, −0.17414, −0.17403, 0.1738, −0.17358, 0.17349, 0.17322, 0.17319, −0.17314, 0.17308, 0.17307, 0.17299, 0.17288, 0.17271, −0.1727, −0.17268, −0.17267, −0.1723, −0.17225, 0.1722, −0.1721, 0.17206, 0.17202, 0.17201, −0.17193, 0.17177, 0.1717, −0.17169, 0.17168, 0.17157, −0.17152, 0.17149, 0.17124, 0.17121, 0.17117, −0.17113, −0.17113, −0.17112, −0.17111, 0.17099, 0.17096, −0.1709, 0.17086, −0.17077, 0.17075, 0.17058, −0.17048, −0.17038, 0.17037, 0.1703, 0.17029, 0.17028, −0.17013, 0.16995, −0.16991, −0.16988, −0.16985, −0.16981, 0.16972, 0.16956, 0.16954, −0.16951, −0.16943, 0.16938, −0.16925, 0.16911, −0.16908, 0.16899, 0.16871, 0.1687, 0.16856, 0.16853, 0.16849, 0.16843, −0.16825, 0.16809, 0.16808, −0.16807, 0.16792, −0.16787, 0.16779, −0.16764, 0.16755, −0.16752, 0.16745, 0.16733, 0.1672, 0.1671, −0.16709, −0.16701, 0.16699, 0.16696, −0.16696, −0.1669, 0.16679, −0.16653, 0.16653, −0.16646, −0.16642, −0.16636, −0.16633, −0.16628, −0.16625, 0.16617, −0.16605, 0.16595, −0.16582, −0.16562, −0.16558, 0.16551, 0.16549, −0.16546, −0.16537, −0.16525, 0.16525, −0.16522, −0.16507, 0.16491, 0.16491, −0.16484, −0.16476, 0.16474, 0.16466, −0.16461, −0.16448, 0.16447, 0.16433, −0.1643, −0.16409, 0.16404, 0.16389, −0.16381, −0.1636, −0.16357, −0.1635, −0.16338, −0.16333, 0.16324, 0.16323, 0.1632, −0.16289, −0.16286, 0.16284, 0.16284, −0.16283, −0.16256, 0.16254, 0.1625, −0.16238, 0.16217, −0.16216, 0.16207, 0.16198, 0.16194, −0.16193, 0.1619, −0.16185, 0.16178, 0.16175, 0.16175, 0.16171, −0.1617, −0.16169, −0.16164, −0.16159, 0.16148, 0.16148, 0.16133, −0.1613, −0.16098, 0.16078, −0.16071, 0.16063, −0.16049, 0.16048, −0.16046, 0.16033, −0.16032, −0.16016, 0.16002, −0.15985, 0.15977, 0.15974, 0.15969, −0.15969, 0.15962, 0.15961, −0.1595, 0.15944, −0.15942, −0.1594, 0.15939, 0.15928, −0.15926, −0.15913, −0.15901, −0.15885, 0.15883, 0.15878, 0.15872, −0.15869, −0.15867, 0.15865, 0.15853, 0.15849, 0.15849, 0.15847, 0.15842, 0.15836, −0.15835, −0.15835, 0.15833, 0.15829, 0.15821, 0.15819, −0.15809, −0.15793, −0.15786, 0.15782, 0.15779, −0.15777, −0.15774, 0.15771, 0.15755, −0.15746, −0.15743, 0.1574, 0.1573, 0.15725, −0.15723, −0.15723, 0.15721, −0.15715, −0.15713, 0.15705, −0.15697, 0.15679, −0.15675, 0.15675, 0.15674, 0.15674, −0.1567, 0.15652, −0.15651, −0.15623, 0.15618, −0.15613, −0.15606, 0.15595, 0.15575, 0.15574, 0.15571, 0.1557, −0.15555, −0.15555, 0.15548, −0.15548, 0.15547, 0.15537, 0.15526, −0.15515, 0.15511, 0.155, 0.15499, 0.15483, 0.1548, 0.15479, 0.15476, −0.15476, 0.15472, −0.15471, −0.15461, 0.1546, −0.15457, −0.15454, −0.15447, −0.15445, 0.15438, 0.1543, −0.15421, −0.15414, 0.15411, 0.15411, 0.15409, 0.15391, 0.15374, −0.1537, −0.15366, 0.15358, −0.15357, −0.15353, −0.15347, 0.15333, −0.15317, −0.15317, −0.15316, 0.15302, −0.15293, 0.15291, 0.15289, 0.15279, 0.15268, 0.15252, 0.15249, −0.15238, −0.15232, 0.15222, −0.15203, −0.15172, 0.1517, −0.1516, −0.15151, −0.15135, 0.15133, −0.15132, −0.15126, −0.15125, −0.15111, 0.15096, −0.15091, 0.1509, −0.15081, 0.15074, 0.15072, 0.15071, −0.15069, −0.15064, 0.15059, −0.15051, 0.15049, 0.15043, −0.15043, −0.15042, −0.15039, −0.15034, 0.15025, −0.15019, 0.15016, −0.15015, −0.1501, −0.15005, 0.15, 0.1499, 0.14987, 0.1498, −0.14979, −0.14971, −0.14968, −0.1496, −0.14958, 0.14956, 0.14944, 0.14942, −0.14936, −0.14934, 0.14925, 0.14923, −0.14919, −0.14918, 0.14911, −0.14906, 0.14883, −0.14859, 0.14856, 0.1485, −0.14847, 0.14845, 0.14831, 0.14824, −0.14823, 0.14822, 0.14803, −0.148, 0.148, −0.14797, 0.14792, 0.14791, 0.14784, 0.14783, 0.14774, −0.14768, −0.14763, −0.14746, 0.14746, −0.14745, −0.14742, 0.14738, −0.14734, −0.14733, 0.1473, −0.14719, −0.14716, −0.14713, 0.14705, 0.14696, −0.14695, −0.1469, 0.14673, −0.14663, −0.14661, −0.1466, −0.14659, 0.14657, 0.14639, −0.14638, −0.14633, 0.14621, 0.1461, 0.146, 0.14597, 0.14589, −0.14586, 0.14581, 0.14578, −0.1457, 0.14562, 0.14555, 0.1454, 0.1454, −0.14538, −0.14534, −0.14527, −0.14515, 0.1451, −0.14509, 0.14495, 0.14488, −0.14487, −0.14469, 0.14465, −0.14443, 0.1444, −0.14438, 0.14438, −0.14434, 0.1442, 0.14419, 0.14415, 0.14409, 0.14404, −0.14402, 0.144, −0.14397, 0.14365, −0.14363, −0.14356, −0.14354, 0.14346, −0.14331, −0.14328, 0.14318, 0.14315, 0.14299, −0.14286, −0.14279, 0.14279, 0.14276, −0.14276, 0.14274, −0.14266, 0.14261, −0.1426, 0.14254, 0.14251, −0.14248, 0.14229, −0.1422, 0.14207, −0.14203, 0.14198, 0.14194, 0.14184, 0.14184, 0.14181, 0.14169, 0.14167, 0.14165, 0.14163, 0.14161, −0.14151, −0.14146, 0.14145, 0.14138, 0.1412, 0.14117, 0.14115, 0.14111, −0.14109, −0.14107, 0.14104, 0.14099, 0.14097, −0.14092, 0.14083, −0.14079, 0.14061, 0.14058, −0.14056, 0.14054, 0.1405, 0.14046, −0.14042, 0.14037, 0.14033, 0.14031, 0.14023, −0.14019, −0.14003, −0.14003, −0.13999, 0.13996, −0.13971, −0.13968, −0.13961, 0.1396, 0.13958, 0.13945, −0.13937, −0.1393, −0.13929, 0.13928, −0.13924, 0.13901, −0.13901, −0.13889, −0.13882, 0.13873, 0.13871, −0.13865, −0.13861, 0.13855, −0.13854, 0.13851, −0.13851, 0.13843, 0.13838, 0.13835, −0.13833, 0.1383, −0.13828, −0.13825, −0.13823, 0.13823, 0.13787, −0.13786, −0.13785, 0.13783, 0.13771, −0.1376, 0.13755, 0.13755, −0.13754, 0.13752, 0.13748, 0.13746, −0.13738, 0.13726, 0.13721, −0.13721, −0.13719, 0.1371, −0.13704, 0.137, −0.13697, −0.13697, 0.13696, −0.13695, −0.13694, 0.13693, −0.13693, −0.13693, −0.13693, 0.13691, −0.1369, −0.13683, 0.13674, 0.13672, 0.13672, 0.13671, 0.13669, −0.13642, 0.13641, −0.13641, 0.13638, 0.13628, −0.13628, −0.13621, −0.13621, 0.13608, −0.13604, 0.13603, −0.13581, 0.13581, 0.13578, 0.13576, 0.13571, 0.1357, 0.13566, −0.13559, −0.13557, 0.13554, −0.13554, −0.13551, −0.13545, −0.13538, −0.13532, −0.13524, 0.1352, −0.13518, 0.13504, −0.13502, 0.13496, −0.13489, −0.13484, 0.13472, 0.13459, −0.13454, 0.13446, 0.13443, −0.13441, 0.13429, 0.13413, 0.13412, 0.13403, −0.13394, −0.13393, −0.13386, 0.13383, 0.1338, −0.13367, 0.13366, 0.13365, −0.13361, −0.13361, −0.13361, 0.13356, 0.13348, 0.13345, −0.13343, 0.13336, 0.13333, −0.13319, 0.13316, −0.13314, −0.13312, 0.13309, 0.13298, −0.13298, 0.13294, −0.13289, −0.13279, 0.13277, −0.13276, 0.13246, −0.13244, −0.13224, 0.13218, −0.13214, −0.13212, 0.13206, −0.13195, −0.13192, −0.13191, −0.13189, 0.13186, −0.13185, −0.13169, 0.13165, 0.13156, 0.13155, 0.13141, 0.13132, −0.13118, −0.1311, −0.131, 0.13097, −0.13094, −0.13086, −0.13082, −0.1308, 0.13076, 0.13065, 0.13063, −0.1306, 0.13058, −0.13057, −0.13053, −0.1305, −0.13046, 0.13044, 0.13036, 0.13035, 0.13032, −0.1303, −0.13027, 0.13027, 0.13026, −0.13025, −0.13022, 0.1302, −0.13018, 0.13009, −0.13004, −0.12999, −0.12991, 0.12986, 0.12979, −0.12976, 0.12972, 0.12964, 0.12963, −0.12948, −0.12948, 0.12942, −0.12934, −0.12932, −0.1293, −0.12929, 0.12928, 0.12924, 0.12919, −0.12915, −0.12911, 0.12908, −0.12905, 0.129, −0.12899, −0.12895, −0.12882, 0.12879, 0.1287, 0.12865, −0.12856, 0.12847, 0.12847, −0.12844, −0.12828, −0.12826, 0.12825, −0.12823, 0.12819, −0.12816, 0.12814, −0.12812, −0.12809, 0.128, −0.128, 0.12794, −0.1279, −0.12784, 0.12782, −0.12781, −0.12779, 0.12777, 0.12776, 0.12776, −0.12775, −0.12775, 0.12774, 0.12773, −0.12765, −0.12764, −0.12763, 0.1276, −0.12746, −0.12745, 0.12744, −0.1274, 0.1273, −0.12724, −0.12718, 0.12714, 0.12712, −0.12712, 0.12712, −0.12707, 0.12705, −0.12702, 0.12701, −0.12699, −0.12698, 0.12687, −0.12684, −0.12682, −0.12681, 0.12679, −0.12679, 0.12667, −0.12634, −0.12627, −0.12615, 0.12596, 0.12595, 0.12594, −0.12588, −0.12577, −0.12572, 0.12571, −0.12566, 0.12563, 0.12558, 0.12557, −0.12551, −0.12543, −0.12536, −0.12528, −0.12526, −0.1252, 0.12516, −0.12516, 0.12506, 0.12499, 0.12492, −0.1249, 0.12478, 0.12474, −0.12473, 0.12472, −0.12471, −0.12471, 0.12466, −0.12449, 0.12446, −0.12443, −0.12443, −0.12439, 0.12438, −0.12437, 0.12431, −0.12428, 0.12425, −0.12423, 0.12423, −0.12423, 0.12421, 0.12419, −0.12419, −0.12413, 0.1241, −0.12409, −0.12409, 0.12409, −0.12406, −0.12405, −0.12403, 0.12397, 0.12394, −0.12392, 0.12378, 0.12375, −0.12372, −0.12367, −0.12365, −0.12362, −0.12353, 0.12351, −0.12346, −0.12344, −0.12342, −0.12337, 0.12334, −0.1233, −0.12325, 0.12324, 0.12319, 0.12316, −0.12315, −0.12308, 0.12303, 0.12296, −0.12292, 0.12291, 0.12273, −0.12272, 0.12261, 0.12255, 0.12252, 0.12251, 0.1225, 0.12243, 0.1224, −0.12237, −0.12236, −0.12228, −0.12228, 0.12206, −0.12206, 0.12204, −0.122, −0.1219, 0.1218, 0.12166, 0.12158, −0.12153, 0.1215, 0.12149, −0.12149, −0.12143, −0.12142, 0.12142, −0.12135, 0.12132, 0.12129, 0.12129, −0.12128, 0.12127, 0.12127, 0.12123, 0.12115, 0.12114, −0.12114, 0.1211, 0.12107, −0.12097, −0.12092, −0.12085, 0.12081, 0.12076, −0.12075, −0.12073, 0.12069, −0.12066, 0.12065, 0.12065, −0.12062, −0.12059, −0.12054, −0.12054, −0.1205, 0.12049, −0.12046, 0.12046, −0.12036, −0.12036, 0.12035, −0.12029, 0.12025, 0.12022, −0.12021, −0.12016, −0.12012, 0.12009, 0.12006, −0.12005, 0.11996, −0.11994, −0.11987, −0.11984, 0.11982, −0.11982, 0.11971, −0.11955, 0.11951, 0.11945, 0.11942, −0.11942, −0.11941, 0.11928, −0.11927, 0.11919, 0.11914, 0.11913, −0.11911, 0.11895, −0.11884, −0.11883, −0.11882, −0.11882, 0.11881, −0.11871, 0.11869, 0.11867, 0.11864, −0.11859, −0.11858, −0.11854, −0.11853, −0.11851, 0.11843, 0.11843, 0.11832, −0.11825, −0.11819, 0.11817, 0.11816, −0.11813, 0.11812, 0.11809, −0.11802, −0.118, 0.11796, −0.11792, −0.1179, −0.11788, 0.11788, 0.11787, 0.11786, 0.11786, 0.11784, −0.11782, −0.11779, −0.11773, −0.11767, 0.11765, −0.11765, 0.11762, −0.11751, 0.11749, −0.11744, 0.11737, 0.1173, −0.1173, −0.11722, −0.11716, 0.11716, 0.11714, −0.11707, −0.11701, −0.117, 0.11699, −0.11697, 0.11695, −0.11684, −0.11681, −0.1167, −0.11663, 0.11662, −0.11658, −0.11657, 0.11653, −0.11644, −0.11638, 0.11627, 0.11618, −0.11617, 0.11617, −0.11615, −0.11608, 0.11605, −0.1159, −0.11587, −0.11581, −0.11576, −0.11574, −0.11572, −0.11565, −0.11564, 0.11562, −0.11562, −0.11561, −0.1156, −0.11559, −0.11557, −0.11554, 0.11552, 0.11551, −0.11544, 0.11541, −0.11534, 0.11534, 0.11527, −0.11526, 0.11518, −0.11508, 0.11505, 0.11505, 0.11501, 0.11498, 0.11496, −0.11494, 0.11482, 0.1148, −0.1148, −0.11476, 0.11476, 0.11471, −0.11467, −0.11458, 0.11458, −0.11452, 0.11445, 0.11444, −0.11443, −0.11442, 0.1144, −0.11438, 0.11432, −0.11424, 0.1142, −0.11416, −0.11414, 0.11413, 0.11412, 0.11403, 0.11402, 0.11401, 0.11396, 0.11391, −0.11388, 0.11388, 0.11387, 0.11385, −0.11378, −0.11376, −0.11376, −0.11374, −0.11374, 0.11372, 0.11371, 0.11368, −0.11364, −0.11363, 0.11361, 0.11356, −0.11355, 0.11354, 0.11352, 0.11344, 0.11344, −0.11339, 0.11329, 0.11329, −0.11323, 0.11309, 0.11306, 0.11305, −0.11303, −0.11301, 0.113, 0.11295, −0.11294, −0.11293, 0.11291, 0.1129, −0.11288, −0.11286, 0.11283, 0.11283, −0.11283, 0.11282, −0.11282, −0.1128, 0.1128, 0.11279, 0.11275, 0.11274, −0.11274, −0.11273, −0.11271, −0.11268, −0.11266, 0.11259, −0.11256, −0.11252, −0.11251, 0.11251, −0.11241, −0.11239, −0.11239, 0.11237, −0.11236, 0.11234, −0.1122, 0.11217, 0.11201, −0.112, −0.112, 0.11198, −0.11196, 0.11194, 0.11193, 0.11191, 0.11189, −0.11187, −0.11185, −0.11177, 0.11171, −0.11166, −0.11165, 0.11164, −0.11156, −0.11153, 0.11151, −0.1115, −0.11143, 0.1114, 0.1114, 0.11135, 0.11131, −0.11131, 0.1113, 0.11121, 0.11119, 0.11119, 0.11114, 0.11112, −0.11109, −0.11104, 0.11099, 0.11095, −0.11094, 0.11094, 0.11092, −0.1109, 0.1109, 0.11088, 0.11086, −0.11086, −0.11085, 0.11084, −0.11075, 0.11072, −0.11071, −0.11068, −0.11063, −0.11063, 0.1106, 0.1106, −0.11059, 0.11053, −0.11045, 0.11033, −0.11033, −0.11031, −0.11025, 0.11021, −0.11017, 0.11016, 0.11015, 0.11013, 0.11011, −0.11009, −0.11007, 0.11, 0.10994, 0.10988, −0.10988, 0.10987, 0.10984, −0.10984, 0.10977, −0.10976, −0.10973, −0.10971, 0.10969, −0.10965, −0.10964, 0.10963, −0.10962, −0.10962, 0.10961, 0.1096, −0.10956, 0.10949, 0.10944, −0.1094, −0.10939, 0.10937, 0.10934, −0.1093, −0.1093, 0.10925, −0.10924, −0.10923, −0.10922, 0.10916, 0.10909, 0.10908, −0.10906, 0.10902, 0.10901, 0.10897, 0.10891, −0.1089, 0.10889, 0.10887, 0.10885, −0.10881, −0.10876, −0.10876, 0.10874, −0.10872, 0.10872, 0.10867, 0.10866, 0.10866, −0.10865, 0.10864, 0.10863, −0.10862, 0.10861, 0.10857, −0.10853, −0.10842, 0.10833, −0.10833, 0.10828, −0.10828, −0.10826, −0.10822, 0.10822, 0.10816, −0.10812, 0.10812, 0.10807, −0.10801, 0.108, 0.10795, −0.10795, −0.10793, −0.10781, 0.1078, −0.10778, 0.10776, 0.10774, −0.10763, 0.10762, −0.10756, −0.10755, 0.1075, 0.10748, −0.10743, −0.10736, 0.10732, 0.10731, 0.10727, −0.10724, 0.10723, 0.10712, −0.10711, −0.10706, 0.107, −0.1069, −0.10687, −0.10685, 0.10682, 0.1068, −0.10677, 0.1067, −0.10668, −0.10667, −0.10661, −0.10659, 0.10653, 0.10651, 0.1065, −0.10648, 0.10645, −0.10644, −0.10643, −0.10639, 0.10634, 0.10627, −0.10624, 0.10622, −0.10619, −0.1061, 0.10607, 0.10602, 0.10601, −0.106, −0.10598, −0.10596, 0.10591, 0.10587, 0.10584, 0.10582, 0.10577, 0.10577, 0.10575, −0.1057, 0.10559, −0.10557, −0.10556, 0.10552, −0.10546, 0.10545, 0.10543, 0.10543, 0.10539, 0.10538, −0.10538, −0.10537, −0.10537, −0.10534, −0.10534, 0.10533, −0.10527, −0.10524, 0.10524, −0.10522, 0.10519, −0.10519, −0.10514, −0.10511, 0.10509, 0.10505, −0.10501, 0.10499, 0.10496, −0.10491, −0.10487, −0.10487, 0.10486, −0.10485, 0.10485, 0.10482, −0.10479, −0.10478, 0.10472, −0.1047, −0.10469, 0.10465, −0.10465, −0.1046, −0.10453, 0.10449, 0.10449, 0.10447, 0.10443, 0.10442, 0.10427, −0.10424, 0.10422, −0.10421, 0.1042, 0.10419, 0.10419, 0.10418, −0.10412, −0.10406, 0.10404, 0.10402, 0.10401, −0.104, −0.10397, 0.10397, 0.10397, −0.10397, −0.10392, −0.10386, −0.10375, −0.10375, −0.10371, 0.10371, −0.1037, 0.10369, 0.10368, −0.10366, 0.10363, 0.10363, 0.10362, 0.10357, −0.10355, 0.10351, −0.1035, −0.10348, −0.10346, −0.10345, −0.10344, 0.10342, 0.10339, −0.10331, 0.10328, 0.10327, 0.10323, −0.1032, 0.10315, −0.1031, −0.1031, 0.10306, 0.10305, −0.10304, 0.10302, −0.103, 0.10298, −0.10295, −0.10294, 0.10291, −0.10289, 0.10289, 0.10288, 0.10285, −0.10282, −0.10277, −0.10274, −0.10274, −0.10272, −0.10266, 0.10265, 0.10262, −0.10256, −0.10252, 0.10249, 0.10248, 0.10248, −0.10244, 0.10236, −0.10235, −0.10235, −0.10235, −0.10234, −0.10233, −0.1023, −0.10229, −0.10229, 0.10227, 0.10226, −0.10225, −0.10222, −0.10221, 0.10219, −0.10218, −0.10216, −0.10213, −0.10212, −0.10211, −0.10201, 0.10199, 0.10192, 0.1019, 0.10181, 0.10181, 0.10178, 0.10174, 0.10174, 0.10171, 0.10168, 0.10167, 0.10163, −0.10162, 0.10158, 0.10157, −0.10155, −0.10153, 0.10152, −0.1015, −0.10144, 0.10139, 0.10132, −0.10131, 0.1013, −0.10128, 0.10124, −0.10121, 0.10118, −0.10116, 0.10114, 0.10113, 0.10112, −0.1011, 0.10107, 0.10103, −0.101, 0.10098, 0.10097, 0.10095, −0.10094, −0.10093, −0.10086, 0.10081, 0.1008, 0.1008, 0.10075, 0.10074, −0.10072, 0.10071, 0.10062, 0.1006, −0.10045, 0.10045, −0.10043, 0.10042, −0.10041, 0.1004, 0.10037, −0.10035, −0.10025, 0.10024, −0.10023, 0.10023, 0.10017, 0.10013, −0.10011, −0.10009, 0.10009, 0.10008, −0.10008, −0.10004, 0.1, 0.09997, −0.09996, 0.09996, −0.09995, −0.09985, 0.09984, −0.09982, −0.09982, −0.09978, −0.09973, −0.09972, 0.0997, 0.09967, 0.09966, 0.09965, 0.0996, 0.0996, 0.09959, 0.09959, 0.09951, −0.09948, −0.09946, 0.09945, 0.09942, −0.09942, −0.09941, −0.09938, 0.09937, 0.09933, 0.09932, 0.09931, −0.0993, −0.09925, −0.0992, −0.09918, −0.09916, 0.09915, −0.09914, 0.09911, −0.09909, −0.09908, −0.09902, −0.09897, −0.09896, 0.09894, 0.09889, −0.09889, 0.09886, 0.09885, −0.09885, −0.09876, −0.09868, −0.09868, 0.09862, −0.09861, 0.09858, −0.09858, −0.09852, 0.09852, −0.09852, 0.09848, 0.09848, 0.09846, −0.09846, 0.09845, −0.09843, 0.0984, 0.09839, 0.09838, 0.09837, −0.09832, 0.0983, 0.09827, −0.09826, 0.09821, −0.09819, 0.09817, −0.09814, 0.09812, 0.0981, −0.0981, 0.09806, 0.09805, 0.09805, 0.09803, −0.09802, −0.09801, 0.098, −0.098, 0.09798, 0.09797, 0.09794, −0.09792, 0.09789, 0.09788, 0.09784, 0.09783, 0.09782, 0.09781, −0.09779, −0.09779, −0.09777, 0.09776, −0.09775, 0.09775, −0.09775, 0.0977, 0.0977, −0.09769, −0.09767, 0.09766, −0.09765, 0.09761, −0.09761, −0.09756, 0.09752, 0.09748, −0.09746, 0.09743, −0.09742, 0.09739, −0.09738, 0.09735, 0.09734, 0.09733, 0.09728, −0.09727, 0.09722, −0.09722, 0.09719, −0.09717, −0.09714, −0.09712, −0.09708, −0.09707, −0.09706, 0.09698, 0.09693, −0.0969, −0.09689, −0.09687, 0.09685, 0.09685, 0.09673, −0.09673, −0.09672, −0.09671, 0.09668, −0.09666, −0.09663, 0.09662, −0.09661, −0.09658, −0.0965, 0.09636, −0.09632, 0.09629, −0.09628, −0.09627, −0.0962, 0.09619, 0.09617, 0.09616, −0.09615, −0.09614, −0.09611, 0.09611, −0.09609, 0.09608, 0.09607, −0.09606, 0.09603, 0.09603, 0.09596, 0.09593, −0.09593, −0.09591, −0.09586, 0.09586, −0.09585, −0.09584, −0.09582, −0.09579, 0.09577, −0.09576, 0.09573, 0.09572, 0.09572, 0.09571, 0.0957, 0.09569, 0.09564, 0.09562, 0.0956, −0.09558, 0.09558, −0.09555, 0.09555, 0.09555, 0.09553, 0.09553, −0.0955, −0.09548, 0.09543, −0.09542, 0.09541, −0.0954, −0.09537, −0.09536, −0.09536, 0.0953, 0.09529, 0.09528, −0.09528, −0.09528, 0.09526, −0.09523, 0.09523, 0.09523, −0.09521, −0.09521, 0.09516, −0.09512, −0.0951, −0.095, 0.09496, −0.09493, −0.09493, 0.09493, −0.09493, −0.09493, 0.09492, −0.09492, 0.09491, −0.09488, −0.09482, 0.09482, −0.09478, 0.09475, −0.09466, −0.09463, 0.09462, 0.09459, 0.09457, 0.09457, −0.09456, 0.09456, −0.09456, 0.09456, −0.09452, 0.09452, −0.09451, −0.09451, 0.09448, 0.09448, 0.09447, −0.09443, 0.09442, −0.09439, 0.09439, 0.09438, −0.09436, 0.09435, 0.09435, 0.09424, 0.09422, 0.09422, 0.09421, −0.09417, −0.09416, 0.09416, −0.09416, −0.09416, 0.09415, 0.09413, −0.09413, 0.09409, −0.09405, −0.09404, 0.09401, −0.09401, 0.09399, −0.09397, 0.09396, −0.09394, −0.09392, −0.09391, 0.0939, 0.09389, 0.09389, −0.09386, −0.09386, 0.0938, 0.09379, −0.09379, 0.09372, −0.09372, 0.09368, 0.09366, 0.09364, −0.09355, 0.09354, 0.09352, −0.09352, 0.09351, 0.09347, 0.09347, −0.09343, −0.09341, −0.0934, −0.09338, −0.09336, −0.09336, 0.09335, −0.09328, −0.09325, −0.09321, 0.09321, −0.09318, 0.09316, 0.09316, −0.09316, 0.09314, 0.09314, −0.09314, −0.09309, 0.09309, 0.09306, 0.09292, −0.09291, −0.09291, 0.09291, 0.0929, 0.09287, −0.09282, 0.0928, −0.09278, −0.0927, 0.09269, −0.09269, −0.09268, −0.09266, −0.09265, 0.09265, −0.09262, −0.09261, 0.0926, −0.0926, 0.09253, 0.09252, 0.09251, −0.09251, −0.09247, −0.09245, −0.09242, −0.09239, −0.09237, −0.09232, 0.09225, −0.09225, 0.09222, 0.09221, −0.09221, −0.0922, 0.09218, −0.09216, 0.09215, −0.09204, 0.09202, −0.092, −0.092, −0.09199, 0.09196, 0.09195, −0.09194, 0.09193, −0.09193, −0.09189, −0.09186, −0.09183, −0.09179, −0.09176, 0.0917, −0.09168, 0.09166, −0.09166, 0.09165, 0.09165, −0.09164, 0.0916, 0.09158, 0.09154, 0.09153, 0.09153, 0.09147, 0.09145, 0.09144, −0.09143, −0.09143, −0.09136, 0.09131, 0.09128, 0.09126, −0.09126, 0.09125, −0.09125, −0.09119, 0.09118, −0.09118, −0.09117, 0.09114, 0.09111, −0.0911, −0.09108, −0.09108, 0.09108, −0.09106, 0.09103, 0.091, −0.09098, 0.09096, −0.09091, −0.0909, −0.0909, −0.09086, −0.09085, −0.09085, −0.09081, 0.09073, −0.09073, −0.09072, −0.09066, −0.09063, −0.09062, 0.09061, −0.09061, −0.09061, 0.09059, −0.09058, −0.09056, −0.09053, 0.09051, 0.09049, −0.09047, 0.09045, −0.09044, −0.09044, 0.09044, −0.09043, −0.0904, −0.09038, 0.09037, 0.09036, 0.09034, 0.09029, 0.09028, −0.09028, −0.09027, 0.09027, 0.09026, −0.09021, 0.09014, −0.09013, 0.09013, 0.0901, −0.09009, 0.09006, −0.09003, −0.09002, −0.09, 0.08996, 0.08996, 0.08995, 0.08995, 0.08993, 0.08989, −0.08988, −0.08988, 0.08988, −0.08988, 0.08987, 0.08984, 0.08984, −0.08982, 0.08978, 0.08977, −0.08977, 0.08976, 0.08974, −0.08974, −0.08971, −0.08967, −0.08967, 0.08966, 0.08964, 0.08964, 0.08961, 0.0896, 0.08956, −0.08956, 0.08955, 0.08952, −0.08951, 0.0895, −0.08949, 0.08943, −0.08942, −0.08942, 0.0894, −0.08935, −0.08933, −0.08931, −0.0893, 0.08929, 0.08929, 0.08928, −0.08926, −0.08926, 0.08921, −0.08919, −0.08916, 0.08911, −0.08911, 0.08909, −0.08908, −0.08907, 0.08904, −0.089, 0.08898, 0.08898, 0.08893, 0.08893, 0.08893, 0.08888, −0.08886, −0.08885, 0.08885, 0.08883, −0.08881, 0.0888, −0.08879, −0.08874, 0.08873, 0.08872, −0.08871, −0.08869, 0.08867, 0.08863, 0.08862, −0.0886, −0.0886, −0.08859, 0.08858, 0.08857, 0.08855, 0.08854, −0.0885, 0.08847, −0.08846, −0.08845, −0.08845, −0.08845, 0.08843, 0.08843, −0.08842, −0.0884, −0.08839, 0.08839, −0.08838, −0.08837, −0.08833, 0.0883, 0.08828, 0.08828, 0.08827, 0.08822, 0.08821, 0.08821, −0.08812, −0.08811, 0.0881, 0.08808, −0.08807, 0.08806, −0.08804, 0.08802, 0.088, 0.08798, −0.08797, −0.08797, −0.08795, 0.08794, 0.0879, −0.08788, 0.08786, 0.08785, 0.08782, 0.08781, 0.0878, −0.08774, 0.08772, −0.08771, 0.08771, 0.08769, 0.08769, 0.08767, −0.08766, −0.08761, −0.08755, −0.08752, 0.08752, −0.08751, 0.0875, −0.08749, 0.08748, −0.08746, 0.08744, −0.08741, 0.08741, 0.0874, −0.08739, 0.08739, −0.08737, 0.08737, 0.08736, 0.08735, −0.08733, −0.08729, 0.08729, −0.08724, 0.08723, −0.08722, −0.08722, 0.08721, 0.0872, −0.0872, −0.08719, 0.08719, 0.08717, −0.08716, 0.08714, −0.08711, −0.08711, 0.08708, 0.08706, 0.08705, −0.08702, −0.087, −0.08696, 0.08694, 0.08691, −0.0869, −0.08689, 0.08688, 0.08688, −0.08686, −0.08685, −0.08679, −0.08679, 0.08674, 0.0867, 0.0866, −0.08651, −0.08649, 0.08647, −0.08644, −0.08642, 0.0864, 0.08639, 0.08639, −0.08635, 0.08634, −0.08633, 0.08624, −0.08624, −0.08623, 0.08622, −0.08621, 0.0862, −0.08619, 0.08618, −0.08617, −0.08616, 0.08616, −0.08616, 0.08616, −0.08614, 0.08612, −0.08607, −0.08605, 0.08604, −0.08601, 0.086, −0.08598, −0.08597, 0.08595, −0.08594, −0.0859, 0.08589, 0.08587, 0.08587, 0.08583, 0.08582, 0.08581, −0.0858, −0.0858, −0.08576, −0.08576, 0.08576, 0.08575, −0.08573, 0.08567, 0.08565, −0.08564, 0.08563, 0.0856, 0.08556, 0.08556, −0.08554, −0.08551, −0.08549, 0.08549, 0.08548, −0.08548, −0.08544, −0.08542, −0.0854, 0.0854, −0.08539, −0.08539, 0.08536, −0.08533, −0.08533, 0.08531, 0.0853, −0.0853, 0.0853, −0.0853, −0.08528, 0.08523, 0.0852, 0.08519, 0.08517, 0.08516, 0.08516, −0.08514, 0.08512, −0.08512, 0.08512, 0.08507, −0.08505, 0.08504, −0.08502, 0.08501, −0.085, −0.08497, −0.08495, −0.08493, −0.08492, −0.08491, −0.0849, 0.08488, 0.08488, 0.08485, −0.08484, −0.08477, −0.0847, −0.08468, 0.08466, −0.08465, 0.08464, −0.08463, −0.08463, 0.08462, 0.08461, −0.08459, 0.08458, −0.08457, 0.08456, 0.08454, 0.08452, 0.08448, 0.08445, −0.08444, 0.08444, −0.08442, 0.08441, 0.08439, 0.08438, −0.08438, −0.08437, −0.08436, 0.08435, 0.08432, −0.08429, 0.08426, −0.08425, 0.08422, −0.08421, 0.0842, 0.08418, −0.08417, −0.08417, 0.08415, −0.08415, −0.08414, 0.08413, 0.08411, −0.0841, 0.08402, 0.08394, 0.08394, −0.0839, 0.0839, 0.08386, −0.08385, 0.08384, −0.08382, −0.08381, 0.0838, −0.08377, 0.08377, 0.08377, −0.0837, −0.08367, −0.08366, −0.08366, −0.08366, −0.08366, −0.08363, −0.08361, −0.0836, 0.08357, −0.08356, −0.08355, −0.08355, 0.08354, 0.08353, 0.08353, −0.08352, 0.0835, 0.08343, 0.08342, −0.0834, −0.08334, 0.08333, −0.08331, −0.08328, −0.08328, 0.08327, −0.08326, −0.08322, −0.08322, −0.08322, −0.0832, −0.08317, 0.08315, −0.08313, 0.08312, 0.0831, −0.08308, −0.08307, 0.08307, 0.08306, −0.08301, −0.08296, −0.08295, 0.08295, 0.08294, −0.08294, −0.08294, −0.08293, −0.08293, 0.08293, 0.08285, −0.08285, 0.08283, −0.08282, 0.0828, −0.08277, −0.08276, −0.08275, 0.08273, −0.08272, 0.08269, −0.08267, 0.08266, 0.08265, 0.08262, 0.08262, −0.08258, 0.08254, 0.08254, −0.08254, 0.08253, 0.08253, 0.08251, 0.0825, 0.08248, 0.08247, −0.08246, 0.08245, 0.08243, −0.08241, 0.08238, −0.08237, 0.08237, 0.08235, 0.08232, −0.08232, −0.0823, −0.08228, −0.08227, 0.08218, 0.08216, 0.08215, −0.08213, −0.08213, 0.0821, −0.0821, −0.08205, −0.08205, 0.08204, −0.08204, 0.08203, 0.08202, −0.08197, 0.08197, 0.08194, 0.08193, −0.0819, 0.08189, 0.08186, −0.08183, 0.08182, −0.08182, 0.08173, −0.08173, 0.08172, 0.08171, −0.08171, 0.08169, 0.08168, −0.08167, −0.08165, 0.08165, −0.08161, −0.08159, −0.08159, 0.08158, 0.08157, 0.08154, −0.08153, −0.08151, −0.08147, −0.08147, −0.08143, −0.0814, 0.08138, −0.08136, 0.08136, 0.08135, 0.08133, 0.0813, 0.08129, 0.08125, 0.08124, 0.08122, 0.08122, −0.08122, −0.08121, 0.08118, −0.08115, −0.08115, −0.08114, −0.08109, 0.08108, −0.08107, 0.08106, 0.08102, 0.08101, −0.08099, 0.08096, −0.08092, −0.08091, 0.08091, −0.08088, −0.08087, −0.08087, −0.08087, −0.08086, 0.08085, −0.08083, 0.08083, −0.08082, −0.08075, −0.08075, 0.08074, −0.08071, 0.08069, 0.08065, 0.08064, −0.08061, −0.0806, −0.08052, −0.08051, −0.08048, −0.08042, −0.08042, 0.08041, 0.08038, −0.08038, 0.08038, −0.08037, −0.08035, 0.08034, −0.08034, 0.08032, 0.0803, −0.08026, 0.08025, 0.08024, −0.08021, −0.08018, 0.08017, −0.08012, 0.08012, 0.08009, 0.08008, −0.08007, −0.08007, 0.08006, −0.07999, −0.07995, −0.07995, 0.07994, 0.07991, 0.07991, 0.07987, 0.07987, −0.07987, −0.07985, −0.07985, −0.07982, −0.07979, −0.07971, −0.0797, 0.0797, −0.0797, −0.07966, −0.07966, −0.07965, 0.07965, 0.07965, −0.07963, 0.07962, 0.07962, −0.07958, −0.07958, 0.07957, −0.07957, 0.07956, −0.07955, −0.07953, 0.07952, 0.07952, 0.0795, −0.07947, −0.07946, 0.07945, −0.07943, −0.07942, −0.07941, −0.0794, 0.07939, 0.07938, 0.07935, 0.07935, 0.07934, −0.07931, 0.07926, −0.07925, 0.07923, −0.07923, −0.07922, 0.07922, −0.0792, 0.07919, −0.07916, 0.07916, −0.07915, −0.07915, 0.07914, 0.07911, 0.07911, 0.0791, −0.07908, −0.07907, 0.07906, 0.07905, 0.07903, 0.07903, −0.07901, 0.07901, −0.079, 0.07897, 0.07896, −0.07894, 0.07893, 0.07887, −0.07886, −0.07884, −0.07881, −0.0788, 0.07878, 0.07876, −0.07869, 0.07867, −0.07867, 0.07866, 0.07863, −0.07862, 0.07858, 0.07855, −0.07853, −0.07851, 0.07845, 0.07844, −0.07842, −0.07841, −0.07839, 0.07838, 0.07833, 0.07832, 0.07832, −0.07831, 0.07831, 0.0783, 0.0783, 0.0783, 0.0783, 0.0783, −0.07829, 0.07827, 0.07822, −0.07822, −0.07821, −0.07819, −0.07818, −0.07817, 0.07814, 0.07812, 0.07812, −0.07811, 0.07809, 0.07806, −0.07804, 0.07803, −0.07801, 0.078, 0.07799, −0.07798, 0.07797, 0.07795, 0.07793, 0.07793, −0.07792, −0.07786, −0.07785, −0.07784, 0.07781, 0.07781, −0.07779, −0.07778, −0.07776, 0.07774, −0.07773, 0.0777, 0.07766, 0.07765, 0.07765, −0.07758, −0.07756, −0.07754, 0.07753, −0.07749, −0.07748, −0.07747, 0.07744, −0.07743, −0.07742, 0.07741, −0.07741, −0.0774, 0.07739, −0.07739, 0.07738, 0.07736, 0.07735, −0.07735, −0.07733, 0.07732, 0.07731, 0.0773, −0.07728, 0.07725, 0.07725, 0.07723, 0.07722, 0.0772, −0.0772, 0.07719, −0.07717, 0.07716, 0.07716, 0.07715, −0.07712, 0.07707, 0.07707, −0.07704, 0.07703, −0.07701, 0.07701, 0.077, −0.077, 0.07696, −0.07696, −0.07693, 0.07693, −0.0769, −0.07689, 0.07688, −0.07687, −0.07686, −0.07686, −0.07685, −0.07683, 0.07679, 0.07678, 0.07677, 0.07676, 0.07676, 0.07672, 0.07671, −0.0767, 0.07669, −0.07667, −0.07666, −0.07662, 0.0766, 0.07659, −0.07656, 0.07655, −0.07651, 0.0765, −0.07649, 0.07648, −0.07644, −0.07642, −0.07639, −0.07637, −0.07636, −0.07636, 0.07633, −0.07632, −0.07631, 0.0763, −0.0763, −0.07628, 0.07627, −0.07625, −0.07619, −0.07616, 0.07613, 0.0761, −0.07608, −0.07605, 0.07604, −0.07604, 0.07604, −0.07599, −0.07599, −0.07598, −0.07596, −0.07594, −0.07592, −0.0759, 0.07589, −0.07587, 0.07587, −0.07587, −0.07586, −0.07585, −0.07583, −0.07583, 0.07582, 0.07582, −0.0758, 0.0758, −0.07577, −0.07576, 0.07576, 0.07575, −0.0757, 0.07569, 0.07569, 0.07568, −0.07568, 0.07567, −0.07566, 0.07566, 0.07564, −0.07563, −0.07561, −0.07555, 0.07555, −0.07554, −0.07552, −0.07551, 0.07547, −0.07546, −0.07545, −0.07542, −0.07538, −0.07537, −0.07535, 0.07533, −0.07532, 0.07531, 0.07528, 0.07527, 0.07524, −0.07523, 0.07522, −0.0752, 0.07519, −0.07511, −0.0751, 0.0751, 0.07508, −0.07507, 0.07507, 0.07506, −0.07506, −0.07504, −0.07498, 0.07496, −0.0749, 0.07489, 0.07488, 0.07488, 0.07483, 0.07482, −0.0748, 0.07479, −0.07478, −0.07473, 0.07472, 0.0747, −0.07468, 0.07467, −0.07467, 0.07466, −0.07466, −0.07462, −0.07461, −0.07461, 0.07459, 0.07459, −0.07457, 0.07457, −0.07456, 0.07455, 0.07454, −0.07454, −0.07452, −0.07452, −0.07451, 0.07451, −0.0745, −0.0745, 0.07446, −0.07443, −0.07441, 0.07439, −0.07439, 0.07438, 0.07438, 0.07437, −0.07436, 0.07436, 0.07436, 0.0743, −0.0743, −0.0743, 0.07428, −0.07425, −0.07425, −0.07422, −0.07422, 0.07418, −0.07416, 0.07416, 0.07416, 0.07413, 0.07411, 0.07411, −0.07409, −0.07409, −0.07409, −0.07409, −0.07408, −0.07407, 0.07407, −0.07404, −0.07404, −0.07403, −0.07402, 0.07402, −0.07401, 0.074, 0.07398, 0.07395, 0.07395, 0.07394, 0.07391, −0.0739, 0.07388, 0.07388, −0.07388, −0.07387, −0.07387, 0.07386, 0.07384, −0.07381, −0.07376, 0.07374, 0.07373, −0.07372, −0.0737, 0.0737, 0.07369, 0.07367, −0.07364, 0.07361, −0.07361, −0.07359, −0.07359, 0.07357, 0.07357, 0.07356, −0.07355, 0.07354, −0.07353, −0.07349, 0.07345, −0.07343, −0.07341, −0.0734, −0.0734, 0.07339, −0.07339, 0.07338, 0.07336, −0.07336, 0.07335, 0.07335, −0.07334, −0.07331, 0.07329, −0.07329, −0.07328, 0.07326, −0.07326, −0.07325, −0.07324, −0.07316, 0.07315, 0.07313, −0.07313, −0.07309, −0.07309, −0.07308, −0.07306, −0.07306, 0.07306, 0.07305, −0.07305, −0.073, 0.073, −0.07299, 0.07298, −0.07294, −0.07293, 0.07293, 0.07291, 0.07289, 0.07289, 0.07288, 0.07286, 0.07281, 0.0728, −0.07279, 0.07278, −0.07278, −0.07276, 0.07271, 0.07269, 0.07267, 0.07267, −0.07263, −0.07262, −0.0726, 0.07259, 0.07259, −0.07258, 0.07256, −0.07252, −0.07251, −0.0725, 0.07247, −0.07246, 0.07246, 0.07246, −0.07245, −0.07243, 0.07242, −0.07242, 0.0724, 0.0724, −0.07236, −0.07235, 0.07235, −0.07234, −0.07233, −0.07231, −0.07231, −0.07231, −0.07231, 0.0723, 0.0723, −0.07229, −0.07226, −0.07226, −0.07222, −0.07222, −0.07221, 0.07221, −0.07221, −0.07219, −0.07219, −0.07217, 0.07215, −0.07214, −0.07214, −0.07213, 0.07212, 0.07208, 0.07207, 0.07206, −0.07203, −0.07202, −0.07202, −0.07202, −0.07198, −0.07197, 0.07195, −0.07195, −0.07193, −0.07192, 0.07192, 0.0719, 0.07187, 0.07187, 0.07186, 0.07185, 0.07182, −0.07182, 0.07179, −0.07179, −0.07178, 0.07176, −0.07174, 0.07169, 0.07169, 0.07168, 0.07168, −0.07164, −0.0716, −0.07159, 0.07157, 0.07155, −0.07154, −0.07154, 0.07154, 0.07154, −0.07152, 0.07151, −0.0715, 0.0715, 0.07149, 0.07149, −0.07149, 0.07146, 0.07146, 0.07146, −0.07146, −0.07145, −0.07144, 0.07144, 0.07142, 0.0714, −0.07139, 0.07137, −0.07137, −0.07137, −0.07137, −0.07135, −0.07135, 0.07133, 0.07131, −0.07129, −0.07129, −0.07126, −0.07125, −0.07124, −0.07122, 0.07118, 0.07118, 0.07118, 0.07117, 0.07117, −0.07116, −0.07116, 0.07116, −0.07115, −0.07114, −0.07114, −0.0711, −0.07109, −0.07109, −0.07108, 0.07105, −0.07104, 0.07103, −0.07101, 0.07097, −0.07097, 0.07097, 0.07095, −0.07095, 0.07095, −0.07094, −0.07093, 0.0709, −0.07089, 0.07087, −0.07086, −0.07083, 0.07082, −0.07082, 0.0708, 0.07079, 0.07078, 0.07076, 0.07075, −0.07075, 0.07072, −0.07071, −0.07067, 0.07067, 0.07066, 0.07066, −0.07065, −0.07064, −0.07064, −0.07063, −0.07061, −0.07061, 0.07059, −0.07058, 0.07057, −0.07057, 0.07053, −0.07052, −0.07051, −0.0705, −0.07047, 0.07047, 0.07046, −0.07044, −0.07044, −0.07043, −0.07042, 0.07041, 0.07041, −0.0704, 0.07039, −0.07039, −0.07038, −0.07037, −0.07033, −0.07033, −0.07031, −0.07031, 0.07031, −0.0703, −0.07028, 0.07027, −0.07026, 0.07026, 0.07025, −0.07024, 0.07021, −0.0702, 0.07018, 0.07017, 0.07015, −0.07015, 0.07013, 0.07012, 0.07012, −0.0701, −0.0701, 0.07009, 0.07009, 0.07005, 0.07004, −0.07002, −0.07002, −0.07002, 0.07001, 0.07, −0.07, −0.07, −0.06995, −0.06994, −0.06993, 0.06993, 0.06992, 0.06992, −0.06992, 0.06991, 0.06987, −0.06985, −0.06985, 0.06985, 0.06984, −0.06984, 0.06982, −0.06981, −0.06981, 0.06981, −0.06981, 0.0698, 0.06978, −0.06977, 0.06976, −0.06975, −0.06975, −0.0697, 0.06968, 0.06967, 0.06967, 0.06967, −0.0696, −0.06959, 0.06957, 0.06956, 0.06955, −0.06955, −0.06953, −0.06953, −0.06951, −0.06949, −0.06947, 0.06944, 0.06943, −0.06942, −0.06941, −0.06941, −0.0694, 0.06937, 0.06936, −0.06935, −0.06934, 0.06933, 0.06931, −0.06931, 0.06931, −0.06929, 0.06929, −0.06928, −0.06927, −0.06925, 0.06923, −0.06922, −0.06919, −0.06913, −0.06913, −0.06912, 0.06911, 0.06907, −0.06905, 0.06903, 0.06902, 0.06899, 0.06896, −0.06896, −0.06894, 0.06893, −0.06893, 0.06893, 0.06891, 0.06891, 0.06889, −0.06889, −0.06888, −0.06888, −0.06887, 0.06886, −0.06882, 0.06881, 0.0688, 0.0688, 0.0688, −0.06876, −0.06876, −0.06875, 0.06874, −0.06869, 0.06868, −0.06864, 0.06863, 0.06863, 0.06862, 0.06859, 0.06859, −0.06856, −0.06855, 0.06854, −0.06849, 0.06849, 0.06847, 0.06846, 0.06844, −0.06844, 0.06843, 0.06842, 0.06841, 0.06841, 0.06836, −0.06836, 0.06834, −0.06829, −0.06827, −0.06825, −0.06823, 0.06822, −0.0682, −0.0682, 0.06819, −0.06819, 0.06819, −0.06818, 0.06817, −0.06815, 0.06813, 0.06813, 0.06812, 0.0681, −0.06808, 0.06808, 0.06806, 0.06804, −0.06804, 0.06803, −0.06803, −0.06803, −0.06802, −0.06801, −0.06799, 0.06798, −0.06795, −0.06795, −0.06792, 0.0679, 0.0679, −0.06789, −0.06789, 0.06789, −0.06786, 0.06786, −0.06785, 0.06783, 0.06782, −0.06782, 0.06782, −0.06781, 0.06781, −0.0678, 0.06779, 0.06779, 0.06778, −0.06778, 0.06778, 0.06777, 0.06774, 0.06774, 0.06771, −0.0677, −0.06769, 0.06765, 0.06764, −0.06763, 0.06758, −0.06754, 0.06751, 0.06751, 0.0675, 0.0675, −0.06749, 0.06749, 0.06748, 0.06747, −0.06746, 0.06746, −0.06743, −0.06742, 0.06741, 0.0674, 0.06739, 0.06738, 0.06736, 0.06735, −0.06734, −0.06734, 0.06733, −0.06732, 0.06731, −0.06731, −0.06731, −0.0673, −0.06729, 0.06728, 0.06727, −0.06726, 0.06722, 0.0672, −0.06719, 0.06719, 0.06718, −0.06714, −0.06714, 0.06713, −0.0671, −0.06709, −0.06708, −0.06706, 0.06705, 0.06702, 0.06701, −0.06701, 0.067, 0.067, −0.06698, −0.06696, −0.06694, 0.06694, 0.06692, 0.06691, 0.0669, −0.06688, 0.06688, −0.06688, −0.06687, 0.06685, 0.06685, −0.06683, 0.06679, 0.06675, 0.06675, 0.06675, −0.06674, −0.06673, 0.06672, 0.0667, −0.06667, 0.06663, −0.06663, 0.06662, −0.06661, −0.06659, 0.06658, −0.06652, −0.0665, −0.06649, 0.06649, 0.06648, 0.06647, 0.06643, 0.06642, −0.06641, −0.06639, −0.06638, −0.06636, 0.06632, −0.06631, 0.06629, 0.06626, 0.06626, −0.06624, 0.06624, 0.06622, −0.06622, 0.0662, 0.0662, 0.06618, 0.06616, −0.06615, 0.06615, −0.06614, 0.06613, −0.06608, −0.06604, −0.06604, 0.06604, −0.06602, −0.06601, 0.06601, 0.06601, −0.066, −0.066, −0.06599, 0.06599, 0.06597, 0.06596, 0.06596, 0.06595, −0.06594, 0.06594, 0.06594, −0.06592, −0.06591, 0.0659, −0.06589, 0.06589, 0.06589, −0.06588, −0.06586, 0.06586, −0.06583, −0.06583, −0.06582, 0.06581, 0.06581, 0.06579, 0.06578, 0.06577, −0.06577, −0.06577, 0.06577, −0.06576, 0.06575, −0.06575, 0.06574, 0.06573, 0.06572, 0.06572, 0.06571, 0.06569, −0.06569, −0.06568, 0.06568, 0.06567, −0.06567, −0.06562, 0.06562, 0.0656, 0.06559, 0.06559, 0.06558, 0.06557, −0.06555, 0.06555, 0.06554, 0.06552, −0.0655, 0.0655, 0.06549, 0.06549, 0.06548, 0.06548, −0.06547, −0.06547, 0.06546, −0.06542, 0.06542, −0.06541, 0.06539, 0.06539, −0.06535, −0.06531, 0.0653, 0.06528, −0.06526, 0.06525, −0.06519, −0.06516, −0.06514, 0.06513, 0.0651, −0.06509, 0.06508, −0.06506, 0.06506, −0.06506, 0.06505, −0.06503, 0.06501, −0.065, −0.065, 0.06498, −0.06497, −0.06497, 0.06496, 0.06496, −0.06492, −0.0649, 0.0649, −0.06489, 0.06489, −0.06488, −0.06487, 0.06484, 0.06484, −0.06484, −0.06483, −0.06482, 0.06482, 0.0648, −0.0648, −0.06479, 0.06478, 0.06477, −0.06475, −0.06473, −0.06472, −0.06472, −0.06471, −0.0647, 0.0647, −0.06469, 0.06467, −0.06466, −0.06464, 0.06463, 0.06463, −0.06462, 0.06462, 0.06462, −0.06462, 0.06461, 0.06461, −0.06457, −0.06455, 0.06455, 0.06454, 0.06454, 0.06452, −0.06452, −0.06452, 0.06451, −0.06449, −0.06447, −0.06446, 0.06445, −0.06445, 0.06443, −0.06441, 0.06441, −0.06441, −0.06437, −0.06435, 0.06435, −0.06435, 0.06434, 0.06431, 0.06429, 0.06428, −0.06427, −0.06426, −0.06425, −0.06423, 0.06423, 0.06423, 0.0642, 0.06415, 0.06412, −0.0641, −0.0641, −0.0641, −0.0641, 0.0641, 0.06409, 0.06407, 0.06407, −0.06407, −0.06407, −0.06406, −0.06404, −0.06404, 0.06402, −0.06401, 0.064, 0.064, 0.06397, −0.06396, 0.06391, 0.0639, −0.06387, 0.06387, −0.06387, 0.06386, 0.06385, 0.06385, −0.06384, −0.06383, −0.06383, 0.06382, −0.0638, −0.06379, 0.06379, −0.06378, 0.06377, −0.06377, −0.06376, 0.06376, 0.06376, −0.06375, −0.06374, −0.06374, 0.06374, −0.06372, −0.06371, −0.0637, 0.06368, 0.06367, −0.06366, −0.06366, 0.06364, −0.06363, 0.06362, 0.0636, 0.06358, −0.06358, 0.06357, 0.06357, 0.06357, 0.06357, −0.06355, −0.06354, 0.06353, −0.06351, −0.06351, 0.06351, −0.06351, −0.0635, 0.06349, 0.06343, −0.06343, 0.06339, 0.06334, −0.0633, −0.06326, −0.06325, −0.06323, −0.06323, 0.06321, 0.06319, 0.06319, 0.06318, 0.06318, −0.06316, 0.06316, 0.06315, 0.06313, 0.06312, 0.06311, −0.06309, −0.06309, −0.06309, 0.06308, −0.06305, −0.06304, 0.06304, 0.06303, 0.06301, −0.063, −0.06297, 0.06294, −0.06292, 0.06292, 0.0629, −0.06289, −0.06288, 0.06287, −0.06286, −0.06285, −0.06284, −0.06283, −0.06282, 0.06281, 0.06281, 0.0628, −0.0628, 0.06279, 0.06279, 0.06279, 0.06279, 0.06276, 0.06276, 0.06275, −0.06274, −0.06272, −0.06272, −0.06268, −0.06267, −0.06267, 0.06266, −0.06265, −0.06265, 0.06265, 0.06264, −0.06264, 0.06264, −0.06264, 0.06263, −0.06262, 0.06261, −0.06258, 0.06258, −0.06258, 0.06254, −0.06254, −0.06253, −0.0625, −0.06249, 0.06248, −0.06246, −0.06246, 0.06244, −0.06243, 0.0624, 0.06239, −0.06239, −0.06238, 0.06234, −0.06233, −0.06231, −0.06228, 0.06228, 0.06228, 0.06227, −0.06219, −0.06218, −0.06217, 0.06217, 0.06216, −0.06216, 0.06216, −0.06214, 0.06213, −0.06213, −0.06212, 0.06211, 0.06211, −0.06209, 0.06208, −0.06208, −0.06207, −0.06206, 0.06206, 0.06205, 0.06203, 0.06202, 0.06202, 0.06201, 0.062, −0.06198, 0.06195, 0.06195, −0.06194, 0.06192, 0.06192, 0.06192, −0.06191, 0.06191, 0.06189, 0.06189, 0.06189, 0.06188, 0.06185, 0.06185, 0.06185, −0.06185, −0.06182, −0.0618, −0.06177, 0.06176, 0.06175, −0.06175, −0.06175, −0.06174, −0.06173, 0.06172, 0.06172, −0.06172, −0.06171, −0.0617, 0.0617, −0.06168, 0.06167, 0.06167, 0.06165, −0.06165, −0.06164, 0.06164, 0.06163, −0.06162, 0.06161, 0.06158, −0.06157, 0.06157, 0.06157, −0.06156, −0.06155, 0.06153, 0.06152, 0.06152, 0.06151, 0.06151, −0.0615, −0.0615, −0.0615, −0.06149, −0.06149, 0.06145, −0.06144, 0.06143, 0.06142, −0.06142, 0.0614, −0.0614, −0.06138, 0.06138, −0.06137, 0.06137, 0.06136, −0.06134, −0.06132, 0.06128, −0.06124, 0.06123, −0.0612, −0.06119, 0.06117, −0.06117, 0.06116, −0.06116, −0.06113, 0.06111, 0.0611, −0.06109, 0.06108, −0.06108, −0.06107, −0.06107, −0.06107, 0.06106, 0.06105, 0.06104, −0.06103, −0.06103, −0.06103, −0.06102, 0.06102, 0.06098, −0.06098, 0.06097, 0.06094, 0.06093, −0.06093, −0.06093, −0.06093, 0.06093, −0.06092, −0.06092, 0.06092, −0.0609, 0.06088, 0.06088, −0.06087, 0.06085, 0.06085, 0.06084, −0.06083, −0.06077, 0.06075, −0.06075, −0.06073, −0.06073, −0.06073, 0.06072, 0.06071, −0.06071, 0.06071, −0.0607, 0.06068, 0.06068, −0.06068, −0.06065, 0.06065, −0.06064, 0.06063, −0.06061, 0.0606, −0.0606, 0.06058, 0.06056, 0.06056, −0.06056, 0.06055, 0.06054, 0.06053, 0.06052, 0.06052, 0.06051, 0.06051, 0.06049, 0.06049, 0.06048, −0.06046, −0.06046, −0.06045, 0.06043, −0.06042, 0.06041, −0.0604, 0.06037, 0.06036, −0.06035, 0.06035, −0.06035, −0.06034, −0.06034, 0.06033, −0.06032, 0.06032, 0.06031, −0.0603, −0.06027, −0.06025, −0.06023, −0.06022, 0.06016, 0.06014, −0.06014, 0.06013, 0.06012, 0.06011, 0.0601, 0.0601, 0.06009, −0.06009, −0.06007, −0.06007, −0.06007, −0.06007, −0.06006, −0.06006, 0.06006, −0.06004, 0.06004, 0.06002, 0.06002, −0.05999, 0.05997, −0.05997, −0.05995, −0.05994, 0.05993, −0.05993, −0.05993, 0.05993, 0.05993, 0.05992, 0.05991, −0.0599, −0.05989, −0.05989, 0.05988, 0.05986, −0.05983, 0.05983, −0.05982, −0.05979, 0.05979, −0.05976, 0.05976, −0.05974, −0.05973, −0.05973, 0.05973, −0.05972, 0.05971, −0.05969, 0.05968, 0.05968, 0.05967, −0.05967, 0.05967, 0.05966, −0.05965, 0.05964, 0.05962, −0.05962, 0.05962, 0.05962, 0.05962, −0.05957, −0.05956, 0.05953, −0.05953, 0.05953, −0.05953, 0.05953, 0.05953, −0.05952, −0.05949, 0.05948, −0.05947, −0.05945, −0.05945, 0.0594, −0.05937, 0.05937, 0.05937, 0.05935, 0.05935, 0.05934, −0.05934, −0.05933, −0.05932, −0.05932, 0.05932, 0.05932, −0.0593, −0.0593, −0.05927, 0.05927, 0.05925, 0.05922, 0.05922, 0.05922, 0.05921, −0.05921, −0.05921, 0.05921, 0.0592, 0.0592, −0.05918, −0.05917, −0.05916, −0.05915, 0.05914, −0.05914, 0.05914, 0.05912, 0.05907, −0.05907, 0.05904, −0.05904, 0.05904, −0.05903, 0.05903, −0.05903, 0.05903, 0.05903, 0.05901, 0.05898, 0.05898, −0.05897, −0.05896, 0.05895, 0.05894, 0.05894, 0.05893, 0.05892, −0.0589, −0.05888, 0.05887, −0.05886, −0.05885, 0.05885, −0.05883, 0.05883, 0.05882, 0.05881, −0.05881, 0.05879, 0.05878, 0.05878, −0.05875, 0.05873, 0.05873, 0.05873, −0.05871, 0.0587, −0.05869, 0.05868, 0.05866, −0.05866, 0.05865, −0.05863, 0.05863, 0.05862, 0.05861, −0.05857, 0.05857, −0.05856, 0.05856, −0.05852, 0.0585, 0.05847, −0.05847, −0.05847, 0.05846, 0.05846, 0.05846, −0.05844, 0.05844, 0.05843, −0.05843, −0.05841, −0.05841, 0.05841, −0.0584, −0.0584, −0.0584, −0.0584, −0.05839, 0.05838, −0.05838, −0.05837, −0.05834, −0.05833, 0.05832, −0.05832, −0.05831, −0.05828, 0.05827, 0.05827, 0.05827, −0.05825, −0.05825, 0.05825, −0.05824, 0.0582, 0.0582, 0.05818, −0.05817, 0.05814, 0.05813, −0.05812, 0.05811, 0.05811, 0.0581, −0.05809, −0.05808, 0.05806, −0.05806, −0.05806, 0.05805, −0.05803, 0.05803, 0.05801, −0.058, −0.05798, 0.05798, 0.05797, 0.05797, 0.05797, 0.05796, −0.05796, −0.05795, 0.05795, −0.05793, −0.05793, −0.05791, −0.05789, 0.05788, 0.05787, 0.05786, −0.05785, 0.05784, −0.05783, 0.05782, 0.05781, −0.05779, 0.05778, 0.05778, 0.05777, −0.05776, −0.05776, 0.05775, 0.05774, 0.05773, −0.05772, 0.05772, −0.05771, −0.05771, 0.05771, −0.0577, 0.05769, 0.05768, 0.05768, −0.05768, 0.05768, 0.05768, 0.05766, −0.05763, −0.05763, −0.05763, −0.05763, 0.05762, −0.05761, −0.0576, 0.0576, −0.05759, 0.05758, 0.05757, −0.05756, −0.05755, 0.05754, 0.05754, −0.05754, −0.05753, −0.05752, −0.05749, 0.05749, 0.05748, −0.05748, −0.05747, 0.05746, −0.05746, 0.05746, 0.05745, −0.05743, 0.05742, 0.0574, −0.0574, 0.0574, −0.05739, −0.05738, 0.05738, −0.05737, −0.05737, 0.05735, 0.05734, −0.05733, −0.05733, 0.05731, −0.05728, 0.05727, 0.05727, −0.05727, −0.05726, 0.05725, 0.05725, 0.05724, 0.05723, 0.05723, −0.05722, −0.0572, −0.0572, −0.05719, 0.05719, −0.05718, −0.05717, 0.05716, 0.05716, −0.05716, −0.05714, 0.05714, 0.05713, −0.05712, −0.05712, 0.05712, −0.05711, −0.05711, −0.05711, 0.05711, 0.0571, −0.05709, 0.05708, 0.05707, −0.05707, 0.05706, −0.05706, 0.05706, 0.05701, 0.057, 0.057, −0.05699, −0.05698, −0.05695, 0.05693, −0.05691, −0.0569, −0.0569, 0.05689, −0.05689, −0.05689, 0.05688, 0.05688, −0.05686, 0.05685, 0.05685, 0.05685, 0.05684, −0.05683, 0.05683, −0.05682, −0.05681, −0.0568, 0.05679, 0.05677, 0.05676, −0.05676, −0.05676, 0.05675, 0.05675, 0.05674, 0.05671, −0.0567, 0.05669, 0.05667, 0.05666, −0.05664, −0.05664, −0.05663, 0.05663, 0.05663, 0.05662, 0.0566, −0.0566, −0.05658, −0.05654, 0.05653, −0.05652, 0.05648, 0.05647, 0.05646, −0.05646, −0.05645, −0.05644, −0.05644, 0.05643, −0.05643, −0.05641, −0.05641, 0.0564, −0.0564, −0.05639, 0.05639, −0.05638, −0.05637, −0.05637, −0.05636, 0.05636, 0.05635, −0.05634, 0.05633, 0.0563, 0.0563, 0.05629, −0.05628, −0.05628, 0.05627, −0.05627, −0.05627, −0.05626, 0.05625, 0.05624, 0.05623, 0.05622, 0.05621, 0.0562, 0.0562, 0.05619, 0.05619, −0.05619, 0.05618, −0.05618, −0.05617, 0.05617, −0.05616, 0.05616, −0.05615, −0.05614, −0.05611, 0.05611, 0.0561, −0.0561, 0.0561, 0.05605, −0.05604, 0.05604, 0.05603, 0.05602, −0.05602, 0.056, −0.056, −0.05599, 0.05599, −0.05597, −0.05597, 0.05596, 0.05593, −0.05593, −0.05592, −0.05591, 0.0559, 0.0559, −0.05589, 0.05589, 0.05588, −0.05586, 0.05584, −0.05583, 0.05582, 0.05582, 0.05578, 0.05578, 0.05578, 0.05576, 0.05576, 0.05572, 0.05571, −0.0557, 0.05568, −0.05568, 0.05568, 0.05567, 0.05566, 0.05566, −0.05566, 0.05565, 0.05565, −0.05564, 0.05562, −0.05561, −0.05559, −0.05556, 0.05556, 0.05555, 0.05553, 0.05552, −0.05552, −0.0555, −0.0555, −0.0555, 0.05549, −0.05549, −0.05548, 0.05546, −0.05546, −0.05545, 0.05545, −0.05544, 0.05542, −0.05542, 0.05542, 0.05541, −0.05541, −0.0554, −0.0554, −0.0554, −0.05538, −0.05538, 0.05537, 0.05536, −0.05535, −0.05534, 0.05533, −0.05532, 0.05532, −0.05531, 0.05531, 0.0553, −0.05528, −0.05526, 0.05522, 0.05522, −0.05522, −0.05522, 0.05519, 0.05519, −0.05519, 0.05518, 0.05518, 0.05517, 0.05516, 0.05515, 0.05514, 0.05514, −0.05513, 0.05512, −0.05511, −0.05511, −0.0551, −0.05509, −0.05508, 0.05506, −0.05505, −0.05504, 0.05503, −0.05503, 0.05503, 0.05503, −0.05503, 0.05502, −0.05502, 0.05502, 0.05501, 0.055, 0.055, 0.05499, 0.05498, −0.05498, 0.05498, −0.05495, 0.05495, 0.05494, 0.05493, 0.05493, −0.05492, 0.05492, 0.05491, 0.05491, −0.05491, 0.05491, 0.05489, 0.05489, −0.05489, 0.05488, 0.05488, 0.05487, 0.05486, 0.05486, −0.05485, 0.05483, 0.05483, −0.05482, 0.0548, −0.0548, −0.05478, 0.05478, 0.05476, 0.05474, 0.05474, −0.05473, −0.05472, 0.0547, 0.05469, 0.05468, 0.05466, −0.05466, 0.05465, 0.05464, −0.05462, −0.05462, 0.05461, −0.05461, −0.05458, 0.05457, −0.05457, −0.05455, 0.05455, −0.05454, 0.05454, 0.05452, −0.05452, −0.0545, −0.0545, −0.0545, −0.0545, 0.05449, 0.05448, −0.05447, −0.05446, 0.05446, −0.05445, −0.05445, −0.05445, 0.05444, −0.05444, −0.05443, −0.05442, −0.05442, −0.05439, −0.05436, −0.05434, 0.05434, −0.05433, −0.05433, −0.05432, 0.05429, 0.05429, 0.05428, −0.05428, 0.05426, −0.05425, −0.05424, 0.05424, 0.05424, 0.05423, 0.05423, −0.05423, −0.05423, 0.05422, 0.05422, 0.05421, −0.05421, −0.05419, 0.05419, 0.05416, 0.05416, 0.05415, −0.05414, 0.05414, −0.05412, 0.05412, −0.05411, 0.0541, 0.0541, 0.05407, −0.05405, 0.05404, 0.05402, −0.05402, −0.05402, 0.05402, 0.05401, −0.054, 0.05399, 0.05398, −0.05396, 0.05396, 0.05393, 0.05392, −0.05391, −0.05391, 0.05389, −0.05387, −0.05387, −0.05386, 0.05385, 0.05384, −0.05382, −0.05382, 0.05382, −0.05379, 0.05379, −0.05379, −0.05378, −0.05377, 0.05377, 0.05376, −0.05375, 0.05375, −0.05375, −0.05375, −0.05374, 0.05373, −0.05373, −0.05371, −0.05371, 0.0537, 0.05369, −0.05368, −0.05368, 0.05367, 0.05367, −0.05367, −0.05367, 0.05364, −0.05363, 0.05362, 0.05362, 0.0536, 0.0536, 0.0536, −0.0536, 0.05359, −0.05358, 0.05356, 0.05356, 0.05354, −0.05354, 0.05354, −0.05353, −0.05352, 0.05352, −0.05351, 0.0535, −0.0535, 0.0535, −0.05348, 0.05348, 0.05345, −0.05344, 0.05344, −0.05343, 0.05342, −0.05342, −0.0534, −0.0534, −0.0534, 0.05339, −0.05339, 0.05337, −0.05337, −0.05336, 0.05335, 0.05334, 0.05333, 0.05332, 0.05332, −0.05331, −0.05331, 0.05328, −0.05327, −0.05326, −0.05323, 0.05322, −0.05322, −0.05321, −0.0532, −0.05319, −0.05319, −0.05319, −0.05318, 0.05317, 0.05317, 0.05316, 0.05314, −0.05314, −0.05314, 0.05313, 0.05313, −0.0531, −0.0531, 0.0531, −0.05308, 0.05305, 0.05301, 0.053, −0.053, 0.05299, 0.05298, −0.05297, −0.05297, −0.05295, 0.05295, 0.05295, −0.05294, −0.05293, −0.05293, −0.05292, 0.05291, 0.05291, 0.05291, 0.05291, 0.0529, −0.0529, −0.0529, −0.0529, −0.0529, 0.0529, −0.05289, 0.05285, −0.05284, 0.05283, −0.05282, 0.05281, 0.05279, 0.05278, −0.05278, −0.05277, −0.05276, −0.05276, −0.05275, 0.05274, −0.05273, 0.05272, −0.05271, 0.05269, 0.05267, −0.05266, −0.05266, 0.05265, −0.05265, −0.05264, −0.05264, −0.05264, −0.05263, −0.05262, −0.05262, −0.05261, 0.0526, −0.05259, 0.05259, 0.05258, −0.05258, 0.05258, 0.05258, 0.05258, 0.05257, −0.05257, 0.05256, −0.05255, −0.05255, −0.05255, −0.05253, 0.05253, 0.05253, −0.05252, −0.05252, −0.05252, 0.05252, 0.05252, 0.05252, 0.05252, −0.0525, −0.0525, −0.05246, 0.05245, −0.05244, −0.05243, −0.05243, 0.05242, 0.05242, 0.05241, 0.05241, −0.05241, 0.0524, −0.05238, −0.05237, 0.05237, 0.05234, −0.05234, −0.05234, −0.05233, 0.05233, 0.05232, 0.0523, 0.0523, −0.05229, 0.05229, −0.05228, 0.05228, 0.05228, −0.05227, −0.05226, 0.05226, 0.05224, 0.05224, 0.05222, −0.05222, −0.05221, −0.0522, −0.0522, −0.0522, −0.05219, −0.05218, −0.05218, −0.05218, 0.05216, −0.05215, 0.05215, −0.05215, −0.05214, −0.05213, −0.05213, −0.05213, 0.05212, 0.05209, 0.05209, 0.05205, −0.05204, 0.05204, 0.05203, 0.05203, 0.05203, 0.05202, 0.05201, 0.052, −0.05195, 0.05195, −0.05195, −0.05194, 0.05194, 0.05191, 0.0519, 0.0519, 0.05186, 0.05183, −0.05181, −0.05181, 0.05181, 0.0518, −0.05178, −0.05177, −0.05175, 0.05175, −0.05175, −0.05175, 0.05175, −0.05174, 0.05174, 0.05173, 0.05173, −0.05173, 0.05172, −0.05171, −0.0517, 0.05168, 0.05167, −0.05165, −0.05164, −0.05164, 0.05164, −0.05162, 0.05162, −0.05161, 0.0516, −0.0516, −0.05158, −0.05157, −0.05157, 0.05157, −0.05156, −0.05156, 0.05155, −0.05155, 0.05154, −0.05153, 0.05153, −0.05153, 0.05152, 0.0515, −0.0515, 0.0515, 0.0515, 0.05149, 0.05149, 0.05147, −0.05147, −0.05147, 0.05147, −0.05147, 0.05147, −0.05146, −0.05146, 0.05145, 0.05145, −0.05143, −0.05142, −0.05142, −0.05141, 0.05141, 0.05141, 0.05141, 0.0514, 0.0514, −0.0514, −0.0514, 0.05139, −0.05139, −0.05138, 0.05138, 0.05137, 0.05136, 0.05135, 0.05135, −0.05135, −0.05134, −0.05133, −0.05131, −0.0513, 0.05129, 0.05128, −0.05128, 0.05128, 0.05128, 0.05127, −0.05126, −0.05125, 0.05125, −0.05124, −0.05123, −0.05121, 0.05121, −0.05119, −0.05119, −0.05118, 0.05117, 0.05117, 0.05117, −0.05116, 0.05116, 0.05115, 0.05115, 0.05115, 0.05114, 0.05113, 0.05113, −0.05112, −0.05112, 0.0511, 0.05109, 0.05109, −0.05109, 0.05107, 0.05105, 0.05105, 0.05104, −0.05103, 0.05103, 0.05103, −0.05103, −0.05101, −0.05101, −0.05101, −0.05101, −0.051, 0.051, 0.051, −0.05099, −0.05098, 0.05098, 0.05097, −0.05097, −0.05096, −0.05096, 0.05095, 0.05094, −0.05091, 0.05091, −0.05091, 0.0509, −0.05089, 0.05088, 0.05087, 0.05087, 0.05086, −0.05086, −0.05086, −0.05085, 0.05084, −0.05083, 0.05083, 0.05083, −0.05082, 0.05082, 0.05082, 0.05082, −0.05081, −0.0508, 0.0508, −0.0508, −0.0508, 0.0508, −0.05079, 0.05079, 0.05076, 0.05075, 0.05074, −0.05074, −0.05074, 0.05073, 0.05072, −0.05071, 0.05071, 0.05069, 0.05069, −0.05069, 0.05068, −0.05068, −0.05067, −0.05067, 0.05066, −0.05066, −0.05066, 0.05065, −0.05065, 0.05065, 0.05065, −0.05064, 0.05062, −0.05058, −0.05058, 0.05057, −0.05057, 0.05056, 0.05056, −0.05056, −0.05056, −0.05055, −0.05055, 0.05054, −0.05053, −0.05053, 0.05052, −0.05051, −0.05051, −0.05051, 0.0505, −0.05049, 0.05046, 0.05045, 0.05045, −0.05043, 0.05043, −0.05042, −0.05041, −0.0504, −0.05039, 0.05039, −0.05039, 0.05038, −0.05036, −0.05036, 0.05035, 0.05034, −0.05033, 0.05033, −0.05032, 0.05031, −0.0503, 0.0503, −0.05029, 0.05029, −0.05029, 0.05029, −0.05028, −0.05028, −0.05027, 0.05027, 0.05026, 0.05025, 0.05023, −0.05022, −0.05022, −0.0502, 0.05017, −0.05017, −0.05017, 0.05016, 0.05016, −0.05015, 0.05014, −0.05014, −0.05014, −0.05012, 0.05011, 0.05011, −0.05011, −0.05009, 0.05009, 0.05008, 0.05008, 0.05007, −0.05006, 0.05006, −0.05004, −0.05003, 0.05003, 0.05002, −0.05, −0.05, 0.04997, −0.04996, −0.04995, 0.04994, −0.04994, −0.04994, −0.04991, −0.04991, −0.04991, 0.0499, 0.0499, 0.0499, 0.04989, −0.04989, 0.04989, −0.04989, 0.04987, −0.04984, −0.04984, 0.04984, −0.04984, −0.04982, 0.04981, 0.04981, −0.0498, 0.0498, −0.04979, −0.04979, −0.04978, 0.04977, 0.04977, 0.04977, −0.04976, −0.04975, −0.04975, −0.04974, −0.04973, 0.0497, 0.0497, 0.04969, −0.04965, −0.04965, 0.04965, 0.04964, 0.04963, −0.04963, 0.04963, −0.04962, 0.04961, 0.0496, −0.04958, 0.04957, −0.04957, −0.04956, −0.04956, −0.04954, −0.04953, 0.04953, 0.04952, −0.0495, 0.0495, −0.0495, −0.04949, −0.04949, −0.04949, 0.04948, 0.04946, −0.04946, −0.04946, −0.04945, 0.04944, −0.04943, 0.04941, 0.0494, −0.0494, −0.0494, −0.04936, 0.04935, −0.04931, 0.04928, 0.04928, −0.04928, 0.04927, 0.04927, 0.04926, 0.04926, 0.04926, 0.04924, 0.04923, 0.0492, 0.04919, 0.04919, −0.04919, −0.04918, −0.04918, 0.04918, −0.04918, −0.04916, −0.04915, 0.04913, 0.04912, −0.04909, −0.04909, 0.04908, −0.04907, −0.04907, −0.04907, 0.04906, −0.04906, 0.04905, −0.04905, 0.04904, −0.04903, 0.04903, −0.04902, −0.04902, −0.04901, −0.04901, 0.04901, −0.049, 0.049, −0.04899, 0.04898, −0.04898, −0.04898, −0.04896, −0.04896, −0.04895, −0.04895, 0.04893, 0.04892, 0.04891, 0.04891, −0.04891, −0.0489, −0.0489, −0.04889, 0.04888, −0.04886, −0.04885, 0.04885, −0.04884, −0.04882, −0.04882, 0.04881, 0.04881, −0.0488, −0.04879, 0.04879, 0.04879, 0.04879, −0.04878, −0.04877, −0.04876, 0.04875, −0.04874, 0.04874, 0.04873, 0.04873, −0.04873, −0.04873, 0.04872, 0.0487, 0.04869, 0.04868, −0.04867, −0.04867, −0.04867, 0.04864, −0.04864, 0.04864, −0.04863, −0.04863, 0.04862, 0.0486, 0.04859, 0.04859, −0.04857, 0.04857, 0.04856, 0.04855, 0.04855, −0.04855, −0.04854, −0.04854, 0.04853, 0.04853, −0.04853, −0.04853, 0.04851, −0.04851, 0.04851, 0.0485, 0.0485, 0.04849, 0.04848, 0.04847, −0.04847, −0.04846, 0.04845, −0.04845, −0.04844, 0.04844, 0.04842, 0.04842, 0.04841, −0.0484, 0.04838, −0.04838, 0.04838, −0.04837, −0.04835, 0.04834, 0.04834, −0.04832, −0.0483, 0.0483, −0.04829, −0.04827, −0.04827, 0.04827, −0.04826, 0.04825, 0.04824, 0.04823, 0.04823, −0.04822, 0.04822, −0.04822, 0.04821, 0.04819, −0.04819, −0.04816, 0.04816, −0.04814, −0.04814, −0.04814, 0.04814, 0.04814, −0.04812, 0.04812, 0.04812, 0.04812, 0.04811, 0.0481, −0.0481, 0.0481, −0.04809, −0.04808, −0.04807, 0.04807, −0.04807, 0.04806, 0.04805, −0.04805, 0.04805, −0.04804, −0.04804, −0.04804, 0.04803, −0.04802, 0.04801, −0.04801, 0.04801, −0.048, 0.048, 0.048, −0.048, −0.04799, −0.04798, 0.04798, −0.04797, 0.04796, 0.04796, 0.04796, −0.04796, 0.04796, −0.04795, 0.04793, −0.04793, 0.04792, 0.04792, −0.04791, −0.0479, −0.04789, −0.04789, 0.04788, 0.04788, 0.04788, −0.04786, −0.04786, 0.04786, 0.04786, 0.04785, −0.04784, −0.04784, −0.04783, −0.04783, −0.04782, 0.04782, 0.04782, 0.04781, 0.0478, −0.0478, 0.0478, −0.04779, 0.04779, −0.04779, −0.04779, 0.04778, 0.04776, 0.04774, 0.04774, 0.04773, 0.04773, 0.04772, 0.04772, −0.04771, −0.04769, −0.04769, −0.04768, −0.04767, −0.04766, −0.04766, −0.04765, −0.04764, 0.04764, −0.04763, −0.04762, 0.0476, −0.04759, 0.04758, 0.04758, 0.04757, 0.04757, 0.04756, −0.04754, 0.04754, −0.04754, 0.04753, −0.04753, 0.04753, 0.04752, 0.04752, −0.04751, 0.0475, −0.0475, −0.04749, 0.04749, 0.04749, −0.04748, −0.04748, 0.04748, −0.04748, −0.04747, 0.04747, 0.04746, 0.04745, 0.04745, 0.04744, 0.04743, −0.04742, 0.04742, 0.0474, 0.04739, 0.04739, 0.04738, −0.04737, −0.04736, 0.04736, −0.04735, 0.04735, 0.04734, −0.04731, −0.04731, −0.04731, 0.0473, 0.04729, 0.04729, 0.04727, 0.04727, 0.04727, 0.04726, −0.04726, −0.04725, 0.04725, −0.04724, 0.04723, 0.04722, −0.04721, −0.04721, −0.0472, 0.0472, −0.0472, −0.0472, 0.0472, −0.04719, 0.04719, 0.04718, 0.04718, −0.04717, 0.04716, 0.04715, 0.04714, −0.04713, 0.04711, −0.0471, −0.04709, 0.04709, 0.04709, 0.04709, −0.04708, −0.04708, −0.04708, −0.04707, −0.04706, −0.04705, 0.04704, −0.047, 0.04699, −0.04698, −0.04697, 0.04696, −0.04695, 0.04694, −0.04694, −0.04694, 0.04693, −0.04692, −0.04691, 0.04691, 0.04691, 0.0469, −0.04689, 0.04689, 0.04688, 0.04688, −0.04687, 0.04686, −0.04686, −0.04683, 0.04683, −0.04682, −0.04682, 0.04681, −0.04681, 0.0468, −0.0468, 0.04679, 0.04676, −0.04676, 0.04676, −0.04676, 0.04675, −0.04675, 0.04674, 0.04674, 0.04674, −0.04673, 0.04673, 0.04672, 0.0467, 0.04669, −0.04669, −0.04669, −0.04668, −0.04667, 0.04667, −0.04667, −0.04667, −0.04666, 0.04665, −0.04664, 0.04664, 0.04663, 0.04663, −0.04662, 0.04662, 0.04662, −0.04661, −0.0466, 0.0466, −0.0466, 0.04659, 0.04658, −0.04658, −0.04658, 0.04658, −0.04657, 0.04656, −0.04656, −0.04655, −0.04654, −0.04653, −0.04653, −0.04651, 0.04651, −0.04649, 0.04648, −0.04648, 0.04647, 0.04646, 0.04646, 0.04646, 0.04642, 0.04641, −0.04641, −0.04641, −0.04641, 0.0464, 0.04637, 0.04637, −0.04635, 0.04632, 0.04631, −0.0463, −0.04628, −0.04627, 0.04627, 0.04626, 0.04625, 0.04625, −0.04624, 0.04624, 0.04623, 0.04622, 0.04619, −0.04619, 0.04619, 0.04618, 0.04618, 0.04617, −0.04617, −0.04617, 0.04617, 0.04617, 0.04617, 0.04617, −0.04616, −0.04615, 0.04614, 0.04614, 0.04614, −0.04613, 0.04613, −0.04612, −0.0461, −0.0461, −0.0461, −0.04608, 0.04608, −0.04607, −0.04607, 0.04607, 0.04607, −0.04607, 0.04607, −0.04605, 0.04605, −0.04604, 0.04602, −0.04602, −0.04602, −0.04602, 0.04601, 0.04599, −0.04599, 0.04598, −0.04598, 0.04596, 0.04596, −0.04594, 0.04593, −0.04592, −0.04591, −0.04591, −0.0459, 0.0459, 0.0459, −0.04589, −0.04589, −0.04588, −0.04588, −0.04587, −0.04587, 0.04587, 0.04587, −0.04587, −0.04587, 0.04586, −0.04584, 0.04583, −0.04583, 0.04583, 0.04581, −0.04581, −0.04581, −0.04581, −0.04581, −0.0458, 0.0458, −0.04579, 0.04578, 0.04576, 0.04576, −0.04576, −0.04575, −0.04575, 0.04575, −0.04575, −0.04572, −0.04572, 0.04572, −0.04572, 0.04571, −0.04571, 0.04571, 0.0457, −0.0457, 0.04569, 0.04569, 0.04569, −0.04569, −0.04569, −0.04568, 0.04567, 0.04566, 0.04565, −0.04565, −0.04564, 0.04564, 0.04564, 0.04563, −0.04563, −0.04562, 0.04562, −0.0456, 0.04559, −0.04559, −0.04557, −0.04556, −0.04554, −0.04553, 0.04553, −0.04551, −0.04551, −0.0455, 0.04548, 0.04548, 0.04547, 0.04546, −0.04545, 0.04544, −0.04544, −0.04544, −0.04543, −0.04542, 0.04542, 0.04542, −0.04542, 0.04542, 0.04541, −0.04541, −0.04541, 0.0454, 0.0454, 0.04539, −0.04538, −0.04538, 0.04537, −0.04536, 0.04535, −0.04535, −0.04533, −0.04531, −0.04531, 0.04531, 0.04528, −0.04528, −0.04527, −0.04527, −0.04527, 0.04526, 0.04526, 0.04525, 0.04525, 0.04524, 0.04524, −0.04524, −0.04524, −0.04523, −0.04521, −0.04521, −0.04521, 0.04519, −0.04519, −0.04519, −0.04519, 0.04515, −0.04515, −0.04514, 0.04514, 0.04513, 0.04512, −0.0451, 0.04509, 0.04508, 0.04507, 0.04506, 0.04505, 0.04504, −0.04502, 0.04502, 0.04502, 0.04502, 0.045, −0.045, 0.04499, 0.04499, −0.04498, −0.04498, −0.04498, −0.04497, −0.04497, 0.04496, 0.04495, −0.04495, 0.04495, 0.04494, −0.04494, −0.04493, −0.04492, 0.04489, 0.04489, −0.04489, −0.04489, 0.04489, −0.04488, 0.04488, 0.04488, 0.04488, −0.04487, 0.04487, −0.04487, 0.04487, −0.04486, −0.04486, −0.04485, −0.04485, −0.04484, 0.04482, 0.04481, 0.04481, −0.04481, 0.0448, −0.0448, −0.04479, 0.04478, 0.04478, −0.04478, −0.04478, 0.04477, −0.04477, −0.04476, 0.04476, −0.04475, −0.04474, −0.04473, −0.04473, 0.04472, 0.04471, −0.04471, 0.04471, −0.0447, −0.04469, 0.04469, −0.04469, −0.04468, 0.04466, −0.04465, 0.04465, −0.04465, 0.04464, −0.04463, −0.04463, 0.04463, −0.04463, 0.04462, −0.04461, −0.04461, −0.04461, 0.04461, −0.04459, −0.04457, −0.04457, 0.04454, −0.04454, −0.04453, 0.04453, 0.04453, 0.04453, −0.04452, 0.04452, 0.0445, 0.04449, 0.04449, −0.04448, −0.04447, −0.04447, 0.04446, −0.04446, 0.04446, −0.04445, 0.04444, 0.04444, 0.04444, −0.04443, −0.04442, −0.04442, −0.04441, 0.0444, −0.0444, 0.0444, −0.0444, 0.04439, 0.04439, −0.04438, 0.04438, 0.04437, 0.04437, −0.04435, 0.04434, −0.04434, −0.04434, −0.04433, 0.04431, 0.0443, −0.04428, 0.04428, 0.04426, 0.04426, −0.04426, −0.04426, 0.04426, −0.04426, 0.04425, 0.04424, 0.04424, 0.04423, 0.04422, 0.04422, 0.04421, 0.04421, −0.0442, −0.0442, −0.0442, 0.04419, −0.04419, −0.04418, −0.04418, 0.04416, −0.04415, 0.04413, 0.04413, 0.04412, 0.04412, −0.04411, 0.04411, −0.04408, 0.04407, −0.04405, −0.04405, −0.04405, −0.04405, 0.04403, 0.04402, 0.04402, 0.04402, −0.04401, 0.044, −0.044, −0.04399, 0.04399, −0.04398, 0.04398, −0.04398, −0.04398, 0.04397, −0.04396, −0.04396, −0.04395, −0.04395, 0.04394, 0.04393, 0.04392, 0.04392, −0.04391, 0.0439, −0.0439, 0.0439, −0.04389, −0.04388, 0.04387, 0.04387, 0.04386, 0.04386, 0.04385, −0.04385, −0.04384, −0.04384, 0.04383, −0.04383, −0.04382, 0.04382, 0.04381, −0.04381, 0.04379, 0.04378, −0.04378, −0.04378, 0.04378, −0.04377, 0.04377, 0.04375, 0.04374, −0.04374, 0.04374, 0.04373, −0.04373, 0.04373, 0.04372, −0.04371, −0.04371, −0.0437, 0.04368, 0.04368, 0.04367, −0.04366, 0.04365, −0.04365, 0.04364, −0.04363, −0.04363, 0.04363, −0.04363, −0.04362, −0.04362, 0.04362, −0.04361, −0.04361, −0.0436, −0.0436, 0.0436, −0.04359, −0.04359, 0.04358, 0.04358, −0.04357, 0.04356, 0.04355, −0.04355, −0.04355, −0.04354, −0.04354, 0.04352, 0.04352, 0.04352, −0.04352, −0.0435, −0.0435, 0.0435, −0.04348, −0.04348, −0.04347, 0.04346, −0.04346, 0.04346, −0.04346, 0.04345, −0.04345, −0.04344, −0.04343, −0.04343, 0.04343, −0.04343, 0.04342, −0.04341, −0.04341, 0.04341, 0.0434, −0.0434, 0.0434, 0.04339, 0.04338, 0.04338, 0.04336, 0.04335, 0.04335, −0.04335, 0.04335, −0.04334, 0.04333, 0.04332, −0.04331, 0.04331, −0.04331, −0.0433, 0.0433, 0.0433, −0.0433, 0.04329, −0.04329, 0.04328, 0.04327, 0.04326, −0.04326, 0.04326, −0.04326, −0.04326, −0.04325, −0.04325, 0.04324, −0.04324, 0.04322, −0.04321, 0.0432, 0.0432, 0.04319, 0.04318, 0.04316, −0.04314, −0.04313, −0.04313, −0.04313, −0.04312, −0.04311, 0.0431, −0.04309, 0.04309, 0.04309, −0.04308, 0.04308, 0.04308, −0.04307, −0.04306, 0.04306, 0.04304, −0.04304, 0.04303, −0.04302, −0.04302, −0.04302, 0.04301, −0.04301, 0.04301, −0.04299, 0.04299, −0.04298, −0.04298, 0.04297, 0.04297, −0.04297, −0.04297, 0.04296, 0.04296, 0.04295, −0.04294, −0.04294, −0.04294, −0.04294, −0.04293, −0.04292, 0.04291, −0.04291, −0.0429, 0.0429, 0.04289, 0.04288, −0.04288, −0.04288, 0.04288, 0.04288, −0.04287, −0.04285, 0.04284, −0.04284, −0.04282, 0.04281, 0.04281, 0.04281, 0.0428, 0.04279, −0.04279, −0.04279, −0.04279, 0.04278, −0.04277, −0.04276, −0.04276, −0.04275, 0.04274, 0.04274, 0.04272, 0.04271, −0.04271, 0.0427, 0.0427, 0.04269, −0.04267, −0.04267, 0.04266, −0.04264, −0.04264, −0.04263, 0.04263, −0.04262, 0.04262, 0.0426, 0.0426, −0.04259, −0.04259, −0.04258, −0.04258, −0.04257, −0.04256, −0.04256, 0.04255, −0.04255, −0.04254, −0.04253, 0.04252, −0.04252, 0.04252, 0.04251, 0.04251, −0.0425, −0.0425, 0.04249, 0.04249, 0.04247, 0.04247, 0.04246, −0.04246, −0.04246, −0.04244, 0.04244, −0.04242, 0.0424, 0.0424, −0.0424, 0.04238, 0.04238, 0.04237, −0.04236, 0.04235, −0.04235, 0.04234, 0.04234, 0.04234, 0.04233, 0.04233, −0.04233, −0.04232, −0.04232, 0.04231, 0.0423, 0.0423, −0.0423, −0.04229, 0.04229, 0.04228, −0.04228, −0.04227, 0.04225, −0.04224, −0.04224, 0.04224, −0.04223, −0.04222, 0.04222, 0.04221, −0.0422, −0.04219, −0.04219, −0.04219, 0.04218, −0.04218, 0.04217, −0.04216, −0.04216, 0.04215, −0.04214, 0.04212, 0.04212, −0.04211, 0.04209, 0.04209, 0.04209, −0.04206, 0.04205, 0.04204, 0.04203, −0.04203, 0.042, 0.042, −0.04199, 0.04199, 0.04199, 0.04199, −0.04199, −0.04197, 0.04194, −0.04194, 0.04193, 0.04193, 0.04193, 0.04192, 0.04192, −0.04191, 0.04191, 0.0419, −0.0419, 0.0419, 0.04189, −0.04189, −0.04189, −0.04189, 0.04188, 0.04187, 0.04186, −0.04184, 0.04184, 0.04183, 0.04182, 0.04182, −0.04181, −0.04181, −0.0418, −0.04179, −0.04179, −0.04179, −0.04179, −0.04178, 0.04178, −0.04178, 0.04177, 0.04177, −0.04176, −0.04176, −0.04175, −0.04175, −0.04175, 0.04173, −0.04173, 0.04171, 0.0417, 0.0417, 0.04169, −0.04168, −0.04167, −0.04167, −0.04166, −0.04166, 0.04166, −0.04165, 0.04165, −0.04165, −0.04165, 0.04165, −0.04164, −0.04164, 0.04163, −0.04162, −0.04162, −0.04161, −0.04161, −0.0416, 0.04159, 0.04159, 0.04158, 0.04158, −0.04158, −0.04157, −0.04156, 0.04156, 0.04155, 0.04155, −0.04154, 0.04154, −0.04153, 0.04152, 0.04152, 0.04151, 0.04151, −0.04147, 0.04147, −0.04146, −0.04145, −0.04145, −0.04144, −0.04144, −0.04142, 0.04141, 0.0414, 0.0414, −0.04139, 0.04139, 0.04138, −0.04138, −0.04137, 0.04137, 0.04135, −0.04134, 0.04133, 0.04133, −0.04133, 0.04132, 0.04132, 0.04131, 0.04131, −0.04129, 0.04129, 0.04127, −0.04126, −0.04125, 0.04125, −0.04125, −0.04125, −0.04125, 0.04124, 0.04124, −0.04124, −0.04123, 0.04123, −0.04122, 0.04121, 0.04121, 0.0412, 0.0412, −0.04119, 0.04119, 0.04118, −0.04118, −0.04117, 0.04117, 0.04116, 0.04116, −0.04115, 0.04115, 0.04115, −0.04114, −0.04114, 0.04113, −0.04113, 0.04113, −0.04113, 0.04112, 0.04111, −0.0411, 0.0411, 0.0411, −0.0411, −0.04107, −0.04107, −0.04106, 0.04105, −0.04105, 0.04104, 0.04103, 0.04102, −0.04102, −0.04101, 0.04101, −0.04101, 0.04101, −0.041, −0.041, 0.04099, −0.04098, 0.04098, 0.04097, 0.04097, 0.04096, 0.04096, −0.04093, −0.04093, 0.04093, 0.04093, −0.04091, −0.04091, 0.0409, −0.0409, −0.0409, −0.04087, −0.04087, 0.04086, 0.04085, 0.04085, −0.04085, 0.04084, 0.04083, 0.04083, −0.04083, 0.04082, 0.04082, 0.0408, −0.0408, 0.0408, 0.04079, −0.04078, 0.04077, −0.04077, −0.04077, −0.04076, −0.04076, 0.04076, 0.04075, 0.04075, 0.04074, −0.04074, 0.04073, 0.04073, 0.04073, 0.04073, 0.04073, −0.04073, −0.04073, −0.04073, −0.04073, 0.04071, 0.0407, 0.0407, −0.0407, 0.0407, −0.04069, −0.04068, 0.04068, −0.04068, −0.04067, −0.04065, 0.04065, −0.04063, 0.04063, 0.04062, 0.04062, −0.04062, 0.04061, −0.04061, −0.0406, −0.0406, −0.04059, 0.04058, 0.04058, −0.04058, 0.04057, −0.04057, 0.04057, 0.04057, 0.04057, −0.04055, −0.04054, −0.04054, −0.04054, 0.04053, −0.04051, 0.0405, 0.0405, −0.04048, 0.04048, 0.04048, −0.04048, −0.04048, 0.04046, 0.04046, 0.04046, 0.04045, −0.04045, 0.04045, 0.04045, 0.04044, −0.04044, −0.04044, −0.04043, −0.04042, 0.04041, −0.04041, 0.04039, 0.04039, −0.04039, −0.04039, −0.04038, −0.04038, 0.04038, 0.04037, −0.04037, −0.04036, −0.04036, 0.04036, −0.04036, −0.04034, 0.04034, −0.04034, −0.04034, −0.04033, 0.04033, −0.04032, −0.04032, 0.04031, 0.04031, −0.04031, −0.0403, −0.0403, 0.0403, 0.0403, −0.0403, 0.04029, −0.04029, 0.04028, −0.04027, 0.04027, −0.04026, 0.04026, −0.04025, 0.04025, −0.04024, 0.04024, −0.04024, −0.04024, −0.04022, 0.04021, −0.04021, −0.04021, −0.04021, 0.04021, 0.04021, −0.04021, 0.0402, −0.04019, −0.04019, −0.04017, 0.04017, 0.04016, −0.04016, −0.04016, 0.04015, 0.04013, −0.04011, 0.0401, −0.0401, 0.04009, 0.04009, 0.04009, 0.04008, −0.04008, 0.04007, −0.04007, 0.04006, −0.04006, 0.04005, 0.04005, 0.04005, −0.04004, 0.04004, −0.04003, 0.04003, −0.04003, −0.04001, 0.04001, −0.04001, 0.04, −0.04, −0.03999, −0.03998, −0.03998, 0.03998, −0.03997, −0.03996, −0.03996, −0.03991, −0.03991, 0.03991, −0.03991, 0.0399, −0.0399, −0.03989, 0.03988, 0.03987, −0.03987, −0.03987, −0.03987, −0.03986, −0.03985, −0.03984, 0.03984, 0.03984, 0.03984, −0.03983, 0.03982, 0.03982, −0.03982, −0.03981, 0.03981, 0.03981, 0.03981, −0.03981, −0.03981, −0.03979, 0.03979, −0.03979, 0.03979, −0.03978, 0.03978, −0.03978, 0.03977, −0.03977, 0.03976, 0.03976, −0.03976, 0.03976, −0.03974, 0.03973, −0.03973, 0.03972, 0.03971, −0.03971, −0.03971, −0.0397, 0.03969, 0.03969, −0.03969, −0.03969, −0.03968, 0.03967, 0.03967, −0.03966, 0.03966, 0.03966, 0.03965, −0.03964, 0.03964, −0.03964, 0.03964, 0.03963, 0.03963, −0.03963, −0.03963, 0.03963, −0.03962, 0.03962, 0.03962, 0.03962, 0.03962, −0.03961, 0.03961, 0.03961, −0.03961, 0.0396, 0.03959, 0.03959, 0.03958, −0.03958, −0.03955, −0.03955, −0.03954, −0.03954, 0.03954, −0.03954, −0.03953, −0.03953, −0.03952, −0.03952, 0.03952, 0.0395, −0.0395, 0.0395, −0.0395, −0.0395, −0.03948, 0.03948, 0.03948, 0.03946, −0.03946, −0.03945, 0.03944, −0.03943, −0.03942, −0.03942, −0.03941, 0.03941, 0.03941, −0.0394, −0.03939, −0.03938, 0.03937, 0.03937, 0.03937, 0.03937, 0.03936, −0.03936, 0.03936, 0.03935, −0.03934, 0.03933, 0.03933, −0.03933, 0.03932, −0.03932, 0.03931, −0.0393, 0.03929, −0.03929, −0.03929, 0.03929, 0.03928, −0.03928, −0.03928, −0.03928, −0.03928, 0.03928, −0.03926, 0.03926, −0.03926, 0.03926, −0.03926, −0.03924, −0.03924, 0.03924, −0.03923, 0.03922, 0.03921, −0.03921, 0.03921, 0.0392, −0.03919, 0.03919, 0.03919, 0.03919, 0.03917, −0.03916, 0.03915, −0.03914, 0.03914, −0.03914, −0.03914, −0.03914, −0.03913, −0.03913, −0.03912, −0.03911, 0.03911, −0.03911, 0.03911, −0.0391, −0.03909, −0.03909, −0.03908, −0.03906, −0.03906, −0.03905, 0.03905, 0.03905, 0.03905, −0.03904, 0.03903, 0.03903, −0.03903, 0.03903, −0.039, 0.039, −0.039, −0.03899, −0.03899, −0.03898, −0.03898, −0.03898, 0.03897, 0.03897, −0.03895, −0.03893, 0.03893, 0.03893, 0.03893, 0.03892, −0.03892, 0.03891, 0.03891, 0.0389, −0.0389, 0.03889, −0.03888, 0.03888, 0.03888, −0.03888, −0.03888, 0.03887, 0.03887, −0.03886, 0.03886, 0.03885, −0.03885, −0.03884, −0.03883, 0.03882, −0.03882, −0.03881, 0.03879, −0.03879, −0.03878, 0.03878, 0.03878, −0.03878, −0.03878, −0.03877, 0.03875, −0.03875, −0.03875, −0.03874, −0.03874, −0.03873, 0.03873, −0.03872, −0.03872, −0.03871, 0.0387, −0.0387, −0.03867, −0.03867, −0.03866, 0.03865, −0.03864, −0.03864, −0.03863, −0.03863, −0.03863, 0.03862, −0.03861, −0.03861, 0.0386, 0.0386, −0.03859, 0.03858, −0.03858, 0.03857, −0.03857, −0.03857, −0.03856, −0.03856, 0.03856, −0.03854, −0.03853, 0.03852, −0.03852, −0.03852, −0.0385, 0.0385, −0.03849, 0.03848, −0.03848, 0.03847, 0.03847, −0.03845, −0.03845, −0.03844, 0.03844, −0.03843, −0.03843, 0.03843, 0.03843, 0.03841, −0.03841, −0.03839, 0.03839, 0.03839, 0.03839, 0.03838, −0.03837, −0.03836, −0.03836, −0.03835, −0.03833, 0.03832, −0.03832, −0.03832, −0.03832, −0.03831, −0.03828, 0.03827, 0.03827, −0.03827, 0.03826, −0.03826, −0.03825, −0.03825, 0.03824, 0.03824, 0.03823, −0.03823, 0.03822, −0.03822, −0.03821, 0.03819, −0.03819, −0.03819, 0.03818, 0.03817, −0.03816, 0.03816, 0.03816, 0.03815, 0.03815, −0.03813, −0.03813, −0.03813, −0.03812, 0.0381, 0.03809, −0.03809, 0.03808, −0.03807, 0.03806, −0.03806, −0.03805, 0.03804, −0.03803, 0.03803, 0.03803, 0.03801, −0.03801, −0.03801, −0.038, 0.03799, 0.03799, 0.03799, −0.03798, 0.03798, −0.03797, 0.03797, 0.03797, −0.03797, −0.03795, 0.03795, −0.03794, −0.03794, −0.03794, 0.03793, −0.03793, −0.03793, −0.03792, 0.03792, −0.03792, 0.03792, 0.03792, −0.03791, −0.03791, 0.03791, −0.0379, 0.0379, 0.0379, 0.0379, 0.03789, 0.03789, 0.03788, −0.03785, 0.03784, 0.03783, −0.03783, 0.03783, −0.03783, −0.03783, 0.03782, −0.03782, 0.03782, 0.03781, −0.03781, 0.03781, 0.03781, 0.0378, 0.0378, 0.0378, −0.03779, 0.03779, −0.03778, −0.03777, 0.03777, 0.03776, 0.03776, 0.03776, 0.03776, −0.03775, −0.03775, −0.03775, 0.03774, −0.03773, −0.03771, 0.03771, −0.03771, 0.0377, −0.0377, 0.0377, −0.0377, 0.03769, 0.03768, −0.03768, −0.03768, 0.03768, 0.03767, −0.03766, −0.03766, 0.03765, 0.03764, −0.03762, −0.03762, −0.03762, 0.03761, 0.03761, 0.03761, −0.0376, 0.0376, 0.0376, 0.03759, −0.03759, 0.03759, 0.03758, −0.03756, −0.03755, −0.03755, −0.03755, −0.03754, −0.03754, 0.03753, −0.03753, 0.03753, −0.03753, 0.03753, 0.03752, −0.03752, 0.03751, −0.03751, −0.0375, 0.0375, 0.0375, −0.0375, −0.03749, 0.03749, 0.03749, −0.03748, 0.03748, −0.03747, −0.03747, −0.03746, 0.03745, −0.03745, 0.03745, 0.03745, 0.03745, −0.03744, 0.03743, 0.03742, 0.03742, 0.03742, 0.03741, 0.03741, −0.0374, 0.03738, −0.03738, 0.03737, −0.03737, −0.03737, −0.03737, 0.03737, −0.03736, −0.03735, −0.03735, −0.03735, 0.03734, 0.03734, −0.03733, 0.03733, −0.03732, −0.03731, 0.03731, 0.03731, −0.03731, −0.03731, 0.0373, 0.03729, −0.03729, −0.03728, −0.03728, 0.03727, −0.03727, −0.03726, 0.03726, −0.03726, −0.03725, −0.03725, 0.03725, −0.03725, 0.03724, 0.03724, 0.03724, 0.03723, 0.03722, 0.03722, 0.03722, 0.03722, 0.03722, −0.03721, −0.03721, 0.0372, 0.03719, −0.03718, 0.03717, −0.03717, 0.03716, −0.03716, 0.03715, 0.03714, −0.03713, 0.03713, −0.03712, −0.03712, 0.03712, 0.03711, 0.0371, 0.0371, 0.03709, −0.03708, −0.03706, −0.03705, 0.03705, −0.03704, −0.03704, −0.03703, −0.03703, 0.03702, −0.03702, −0.03701, −0.03701, 0.03701, 0.037, −0.037, 0.037, −0.037, 0.03699, −0.03699, 0.03698, 0.03698, 0.03698, −0.03698, 0.03697, 0.03697, −0.03697, 0.03697, −0.03696, 0.03695, −0.03695, 0.03694, 0.03694, −0.03694, 0.03694, −0.03693, 0.03692, −0.03692, 0.03691, 0.0369, −0.0369, −0.0369, −0.0369, −0.03688, −0.03688, 0.03687, −0.03687, 0.03687, 0.03686, 0.03686, −0.03686, 0.03686, 0.03685, −0.03685, −0.03685, 0.03684, 0.03684, 0.03683, 0.03683, 0.03682, −0.03682, −0.03681, 0.0368, −0.0368, 0.03678, 0.03678, 0.03678, 0.03678, 0.03677, 0.03677, 0.03676, −0.03675, −0.03675, −0.03675, −0.03674, −0.03673, 0.03673, 0.03672, −0.03672, −0.03671, −0.0367, −0.03668, −0.03668, 0.03667, −0.03667, −0.03666, −0.03666, 0.03666, −0.03665, 0.03665, −0.03665, −0.03665, −0.03665, 0.03664, 0.03664, 0.03664, 0.03663, −0.03663, 0.03662, 0.03662, −0.03662, 0.03662, −0.03662, −0.03661, 0.03661, 0.03661, 0.0366, −0.03659, −0.03659, −0.03658, 0.03658, 0.03658, −0.03658, 0.03657, 0.03657, 0.03656, 0.03656, −0.03654, −0.03654, 0.03654, −0.03654, 0.03654, 0.03654, 0.03653, −0.03653, −0.03653, 0.03653, −0.03652, −0.0365, −0.0365, 0.0365, −0.03649, 0.03649, 0.03648, −0.03647, 0.03647, −0.03647, 0.03645, 0.03645, −0.03644, 0.03643, −0.03643, −0.03643, 0.03643, −0.03642, −0.03642, −0.03642, 0.03642, 0.03641, 0.03641, −0.0364, 0.03639, 0.03639, 0.03639, 0.03638, −0.03638, 0.03637, 0.03637, 0.03636, 0.03636, 0.03636, −0.03636, −0.03635, −0.03635, −0.03634, −0.03633, −0.03633, −0.03633, 0.03633, −0.03632, 0.03632, −0.03632, 0.03632, 0.03632, −0.03632, −0.03632, 0.03631, 0.03631, 0.03631, −0.0363, −0.03629, −0.03629, 0.03629, 0.03629, 0.03628, −0.03627, 0.03627, 0.03626, −0.03624, 0.03624, 0.03624, 0.03624, 0.03622, 0.03621, 0.03621, −0.03621, −0.0362, 0.03619, 0.03619, 0.03618, 0.03618, −0.03618, −0.03618, −0.03617, 0.03616, −0.03616, −0.03615, −0.03615, −0.03615, 0.03613, −0.03613, 0.03613, 0.03612, −0.03611, −0.03611, −0.0361, 0.0361, −0.0361, −0.0361, −0.03609, −0.03609, 0.03609, −0.03608, 0.03608, 0.03607, 0.03606, −0.03606, 0.03606, 0.03605, −0.03605, 0.03605, −0.03605, 0.03604, 0.03604, −0.03603, −0.03603, 0.03603, 0.03603, −0.03602, 0.03602, 0.03602, 0.03599, 0.03599, 0.03598, −0.03598, 0.03597, 0.03596, 0.03596, 0.03595, −0.03594, −0.03593, 0.03593, −0.03593, −0.03593, −0.03592, 0.03592, 0.03592, −0.03592, 0.03592, 0.03591, −0.0359, 0.03589, −0.03589, −0.03589, −0.03589, 0.03589, −0.03589, −0.03589, −0.03588, −0.03587, 0.03586, −0.03586, −0.03585, −0.03585, −0.03585, 0.03584, −0.03584, −0.03584, 0.03584, 0.03583, 0.03582, 0.03581, 0.03581, 0.03581, −0.03581, −0.03581, −0.0358, −0.0358, −0.03579, 0.03579, −0.03578, 0.03578, 0.03578, 0.03577, −0.03577, −0.03576, −0.03576, 0.03576, −0.03575, −0.03575, 0.03575, −0.03574, −0.03573, −0.03573, 0.03573, 0.03573, 0.03572, 0.03572, 0.0357, −0.0357, 0.03569, −0.03569, −0.03569, 0.03568, 0.03568, −0.03568, −0.03568, −0.03566, −0.03565, 0.03564, 0.03564, 0.03564, −0.03564, −0.03564, 0.03563, −0.03562, −0.03561, −0.03561, −0.0356, −0.0356, −0.0356, 0.0356, 0.0356, −0.03559, −0.03559, −0.03559, −0.03557, 0.03557, −0.03556, −0.03555, 0.03553, 0.03553, −0.03552, −0.03552, 0.03551, 0.03551, 0.03551, −0.0355, −0.03549, −0.03549, 0.03549, 0.03548, −0.03548, −0.03548, 0.03547, 0.03547, 0.03546, −0.03546, −0.03545, −0.03543, 0.03543, −0.03542, −0.03542, 0.03542, 0.03541, −0.03541, −0.03541, 0.03541, −0.0354, 0.0354, 0.03539, 0.03539, 0.03539, −0.03537, −0.03537, 0.03537, −0.03536, −0.03536, −0.03535, −0.03535, −0.03534, −0.03534, 0.03534, 0.03533, 0.03533, −0.03533, −0.03533, −0.03533, 0.03532, −0.03532, 0.03532, −0.03531, 0.03531, −0.0353, −0.03529, 0.03529, −0.03528, 0.03528, −0.03528, 0.03527, 0.03526, 0.03526, −0.03526, 0.03526, −0.03526, −0.03525, −0.03524, −0.03524, 0.03523, 0.03522, 0.03521, −0.03521, −0.03521, −0.0352, −0.0352, −0.0352, 0.0352, −0.03519, −0.03518, 0.03518, 0.03516, −0.03515, 0.03515, −0.03515, −0.03515, −0.03514, −0.03513, 0.03513, 0.03512, 0.03512, 0.03511, −0.03511, 0.0351, 0.0351, −0.03509, −0.03509, 0.03509, 0.03509, −0.03509, −0.03508, 0.03508, 0.03508, −0.03508, −0.03508, −0.03508, 0.03507, 0.03507, 0.03506, 0.03506, 0.03506, 0.03505, 0.03505, 0.03505, −0.03504, 0.03504, 0.03503, 0.03503, 0.03501, −0.03501, 0.03501, −0.035, 0.035, 0.03498, −0.03498, −0.03498, 0.03497, 0.03497, −0.03497, −0.03495, 0.03493, 0.03493, 0.03493, −0.03493, 0.03492, −0.03492, −0.03492, −0.03492, 0.03492, −0.03491, −0.03491, 0.03491, −0.0349, 0.0349, −0.03489, −0.03489, −0.03488, 0.03488, 0.03488, 0.03488, 0.03487, 0.03486, −0.03486, −0.03485, −0.03485, 0.03485, 0.03485, −0.03484, 0.03483, 0.03482, −0.03481, 0.03481, 0.0348, 0.0348, 0.0348, −0.03479, 0.03479, −0.03479, 0.03478, −0.03478, −0.03478, −0.03477, 0.03477, 0.03476, 0.03475, 0.03475, −0.03475, 0.03475, 0.03474, 0.03473, −0.03473, 0.03472, −0.03471, −0.03471, −0.03471, 0.03471, −0.03469, −0.03469, −0.03469, 0.03469, −0.03468, 0.03467, 0.03466, 0.03465, −0.03464, 0.03464, 0.03463, 0.03462, −0.03462, −0.03462, −0.03461, 0.0346, −0.0346, −0.0346, 0.03459, −0.03459, −0.03458, 0.03457, 0.03457, −0.03457, 0.03456, 0.03456, 0.03456, 0.03454, −0.03454, −0.03454, 0.03453, 0.03453, 0.03452, 0.03452, 0.03452, −0.03451, −0.0345, 0.03449, −0.03449, −0.03449, −0.03448, 0.03448, −0.03447, 0.03447, 0.03447, −0.03446, −0.03445, 0.03445, −0.03444, 0.03442, 0.03442, −0.03441, 0.0344, −0.0344, 0.03439, 0.03439, 0.03438, 0.03437, 0.03436, −0.03436, −0.03436, −0.03436, −0.03435, 0.03435, 0.03435, 0.03435, −0.03435, −0.03434, 0.03434, 0.03434, −0.03434, −0.03433, 0.03433, −0.03432, 0.03432, 0.03432, 0.03431, −0.0343, 0.0343, −0.0343, 0.03429, −0.03429, 0.03428, −0.03428, 0.03428, 0.03427, 0.03427, −0.03427, −0.03427, 0.03425, 0.03425, −0.03425, −0.03424, −0.03424, −0.03424, −0.03424, 0.03422, 0.03422, 0.03422, −0.03421, −0.03421, −0.0342, 0.0342, 0.0342, 0.0342, −0.0342, 0.03419, −0.03418, −0.03418, 0.03418, −0.03417, 0.03417, −0.03417, 0.03417, 0.03416, 0.03416, 0.03415, −0.03415, 0.03414, −0.03413, −0.03413, −0.03413, −0.03413, 0.03412, 0.03412, −0.03411, −0.03411, 0.03411, 0.03411, −0.0341, 0.03409, 0.03409, −0.03408, −0.03408, −0.03408, −0.03407, −0.03407, −0.03407, −0.03406, 0.03406, 0.03406, 0.03406, 0.03406, −0.03405, 0.03405, −0.03405, −0.03405, −0.03403, 0.03403, 0.03403, −0.03402, 0.03402, 0.03401, 0.03401, −0.034, −0.034, 0.03399, 0.03399, −0.03399, −0.03399, 0.03399, −0.03398, −0.03398, 0.03398, 0.03398, 0.03397, 0.03395, −0.03395, 0.03394, 0.03394, 0.03394, −0.03394, 0.03393, −0.03393, −0.03393, 0.03393, −0.03392, 0.03392, 0.03391, 0.03391, 0.03391, 0.03389, −0.03389, 0.03389, 0.03388, 0.03385, 0.03385, 0.03384, −0.03384, 0.03384, 0.03383, 0.03382, 0.03382, 0.03381, 0.03381, −0.03381, −0.03381, 0.03381, 0.03381, −0.03381, 0.03381, 0.0338, −0.0338, −0.0338, 0.0338, 0.0338, −0.03379, 0.03379, −0.03379, −0.03378, 0.03378, −0.03378, −0.03377, −0.03376, 0.03376, −0.03375, −0.03374, 0.03374, 0.03374, −0.03373, 0.03373, −0.03373, −0.03373, −0.03372, −0.03372, 0.03371, −0.03371, 0.03371, −0.0337, 0.0337, 0.0337, −0.03369, −0.03369, −0.03369, −0.03369, −0.03368, 0.03366, −0.03365, 0.03365, −0.03365, 0.03365, 0.03364, −0.03364, 0.03364, 0.03364, −0.03363, 0.03363, −0.03362, −0.03362, −0.03361, −0.03361, −0.03361, 0.0336, −0.03359, 0.03359, −0.03358, 0.03358, −0.03358, −0.03357, 0.03357, 0.03357, −0.03357, 0.03356, 0.03356, −0.03355, −0.03355, 0.03355, 0.03355, 0.03355, 0.03354, 0.03354, −0.03354, −0.03352, −0.03352, 0.03352, −0.03352, −0.0335, 0.03349, 0.03349, −0.03349, 0.03349, −0.03348, −0.03348, −0.03347, 0.03346, 0.03346, −0.03346, 0.03345, −0.03345, 0.03344, 0.03343, 0.03343, 0.03342, −0.03342, −0.03342, 0.03342, −0.03341, −0.03341, 0.03341, 0.0334, −0.03339, −0.03338, 0.03338, −0.03338, −0.03336, 0.03335, −0.03335, 0.03335, 0.03334, −0.03333, 0.03333, 0.03332, −0.03331, −0.03331, −0.03331, −0.03331, 0.0333, −0.0333, −0.0333, 0.03329, −0.03328, 0.03328, 0.03328, −0.03328, −0.03328, 0.03327, −0.03327, −0.03327, 0.03327, 0.03326, 0.03326, 0.03325, 0.03325, 0.03323, −0.03323, −0.03322, −0.03322, 0.03322, 0.03321, 0.03321, 0.03321, −0.03321, −0.03321, 0.0332, 0.0332, −0.0332, −0.03319, 0.03318, 0.03318, −0.03318, −0.03317, 0.03316, −0.03316, 0.03316, −0.03316, 0.03316, 0.03316, 0.03315, −0.03315, −0.03314, 0.03313, 0.03313, 0.03312, 0.03312, 0.03311, −0.03311, −0.03311, −0.03311, 0.0331, −0.0331, −0.03309, 0.03309, 0.03309, 0.03309, −0.03308, 0.03308, 0.03308, 0.03307, −0.03307, −0.03307, 0.03305, −0.03305, 0.03303, −0.03302, −0.03302, 0.03301, 0.03301, −0.033, −0.033, −0.03299, 0.03299, 0.03299, −0.03299, −0.03299, −0.03299, −0.03299, −0.03298, −0.03298, 0.03298, 0.03298, −0.03297, −0.03297, −0.03296, −0.03295, 0.03294, 0.03294, 0.03294, −0.03294, −0.03294, 0.03293, −0.03293, 0.03292, −0.03292, −0.03292, 0.03292, −0.03292, 0.03291, 0.03291, 0.03291, −0.03291, −0.03291, 0.0329, −0.0329, 0.0329, 0.03289, 0.03289, −0.03289, −0.03289, 0.03288, 0.03288, −0.03288, −0.03287, 0.03287, −0.03286, 0.03286, 0.03286, 0.03285, 0.03284, −0.03284, 0.03284, −0.03284, −0.03283, −0.03283, −0.03283, −0.03283, 0.03282, 0.03282, 0.03282, −0.03281, −0.03281, 0.0328, 0.0328, 0.0328, −0.03279, 0.03279, 0.03279, 0.03278, 0.03278, 0.03277, 0.03277, −0.03277, −0.03276, 0.03275, −0.03275, 0.03274, 0.03274, 0.03273, 0.03272, −0.03272, 0.0327, 0.0327, −0.0327, 0.0327, 0.03269, −0.03269, 0.03268, −0.03268, −0.03268, −0.03266, −0.03266, −0.03266, −0.03266, −0.03265, 0.03265, 0.03265, −0.03265, 0.03265, −0.03264, −0.03264, 0.03263, −0.03263, −0.03262, 0.03262, −0.03261, 0.03261, −0.03261, 0.0326, 0.03259, −0.03259, 0.03259, 0.03259, −0.03259, 0.03258, −0.03258, 0.03258, −0.03258, 0.03258, 0.03258, 0.03258, 0.03257, −0.03257, 0.03256, −0.03256, −0.03255, 0.03255, −0.03254, −0.03254, 0.03253, 0.03253, −0.03253, 0.03252, −0.03252, −0.03251, 0.0325, 0.0325, −0.0325, −0.0325, 0.03249, −0.03249, 0.03249, −0.03248, 0.03248, −0.03247, 0.03247, −0.03247, 0.03246, 0.03246, −0.03246, −0.03245, 0.03244, −0.03243, 0.03243, 0.03243, −0.03242, 0.03242, 0.03241, 0.03241, −0.03241, −0.03241, −0.0324, 0.0324, 0.03239, −0.03239, 0.03239, −0.03239, 0.03239, 0.03237, 0.03237, −0.03237, −0.03237, −0.03237, −0.03237, 0.03237, −0.03236, −0.03235, −0.03235, 0.03233, −0.03233, 0.03233, 0.03233, −0.03232, 0.03232, −0.03232, 0.03232, 0.03232, −0.03231, −0.03231, −0.03231, 0.03231, −0.03231, −0.0323, −0.0323, 0.0323, 0.03229, 0.03229, −0.03228, 0.03228, 0.03228, 0.03228, 0.03227, −0.03227, −0.03226, −0.03226, −0.03225, −0.03225, 0.03225, 0.03225, 0.03225, 0.03225, −0.03224, 0.03224, −0.03224, 0.03224, 0.03224, 0.03222, 0.03222, −0.03222, 0.03221, −0.0322, 0.0322, −0.0322, −0.0322, −0.03219, −0.03219, −0.03218, −0.03218, −0.03218, −0.03218, 0.03218, −0.03217, 0.03217, 0.03216, 0.03215, 0.03214, −0.03214, 0.03214, −0.03214, −0.03214, −0.03214, 0.03213, 0.03212, −0.03212, 0.03211, 0.03211, −0.03211, −0.0321, 0.03209, −0.03209, −0.03209, 0.03209, 0.03209, −0.03209, −0.03208, −0.03207, 0.03207, −0.03205, −0.03205, −0.03205, 0.03205, −0.03204, 0.03204, −0.03203, −0.03202, 0.03202, −0.03202, 0.03201, 0.03201, 0.03199, 0.03199, −0.03199, 0.03199, −0.03198, 0.03198, −0.03197, 0.03196, −0.03196, 0.03195, −0.03195, −0.03194, 0.03194, 0.03193, 0.03193, −0.03193, 0.03192, −0.03192, 0.03191, 0.03191, −0.03191, 0.0319, −0.0319, 0.0319, −0.0319, 0.03189, −0.03189, −0.03189, 0.03189, −0.03188, 0.03187, −0.03187, −0.03187, −0.03187, −0.03186, −0.03186, 0.03186, −0.03186, −0.03184, 0.03184, 0.03183, −0.03183, 0.03183, −0.03183, 0.03182, 0.03182, −0.03182, 0.03182, −0.03181, 0.03179, −0.03179, 0.03178, 0.03177, 0.03176, −0.03175, −0.03175, −0.03175, 0.03174, 0.03174, −0.03174, 0.03173, −0.03172, −0.03171, 0.03171, −0.03171, −0.03171, −0.0317, 0.03169, 0.03169, 0.03169, −0.03168, −0.03168, −0.03168, −0.03168, −0.03167, 0.03167, −0.03167, −0.03166, −0.03166, −0.03165, 0.03165, 0.03165, −0.03165, 0.03164, 0.03164, −0.03164, −0.03164, −0.03164, −0.03163, 0.03163, −0.03162, 0.03161, −0.03161, 0.0316, −0.0316, −0.0316, −0.0316, −0.03159, 0.03159, −0.03158, 0.03158, −0.03158, −0.03158, −0.03158, −0.03157, 0.03157, −0.03157, −0.03156, 0.03156, −0.03156, 0.03155, 0.03155, −0.03155, −0.03155, −0.03154, 0.03154, 0.03154, −0.03154, 0.03154, −0.03153, 0.03153, 0.03153, 0.03153, −0.03153, 0.03152, −0.03152, −0.03152, 0.03152, 0.03151, 0.03151, 0.03151, 0.03151, 0.0315, −0.03149, −0.03149, 0.03149, 0.03148, −0.03147, −0.03147, −0.03147, 0.03146, −0.03146, 0.03146, −0.03146, −0.03146, 0.03145, 0.03145, −0.03145, −0.03145, −0.03145, −0.03144, −0.03144, −0.03144, −0.03143, −0.03143, −0.03143, 0.03143, 0.03142, 0.03142, −0.03142, −0.0314, −0.03139, −0.03139, −0.03139, −0.03139, 0.03139, −0.03138, 0.03138, −0.03137, −0.03137, 0.03137, 0.03136, 0.03135, 0.03135, −0.03132, −0.03131, 0.03131, 0.0313, −0.03129, 0.03129, 0.03129, −0.03128, −0.03127, −0.03127, −0.03127, 0.03126, −0.03126, 0.03125, 0.03125, −0.03124, −0.03124, 0.03124, −0.03123, −0.03123, −0.03123, −0.03123, 0.03122, 0.03122, −0.0312, 0.03119, 0.03119, −0.03119, 0.03119, −0.03118, 0.03118, −0.03117, 0.03117, 0.03117, 0.03116, 0.03115, −0.03115, −0.03115, −0.03115, −0.03114, 0.03114, 0.03114, −0.03113, −0.03113, −0.03113, −0.03113, −0.03112, 0.03112, −0.03112, 0.03112, 0.03111, 0.03111, −0.03111, −0.03111, 0.0311, 0.0311, 0.0311, −0.0311, 0.03109, 0.03108, −0.03108, 0.03108, −0.03107, 0.03106, 0.03106, 0.03106, −0.03106, −0.03105, 0.03105, 0.03105, −0.03103, −0.03103, 0.03103, 0.03102, 0.03101, 0.03101, 0.03101, −0.031, 0.031, 0.031, 0.03099, 0.03099, 0.03099, −0.03099, −0.03098, −0.03098, 0.03098, −0.03097, −0.03097, −0.03097, −0.03097, −0.03096, −0.03096, 0.03096, −0.03096, −0.03095, −0.03094, −0.03094, −0.03094, −0.03094, −0.03094, 0.03093, 0.03093, −0.03093, 0.03093, −0.03092, 0.03092, 0.03092, 0.03091, −0.03091, 0.0309, 0.0309, −0.03089, −0.03089, 0.03089, −0.03088, −0.03088, 0.03087, −0.03087, 0.03087, 0.03087, −0.03086, 0.03086, 0.03086, −0.03085, 0.03085, 0.03085, 0.03084, −0.03084, −0.03083, −0.03082, 0.03082, −0.03082, 0.03081, 0.03081, −0.03081, −0.03081, 0.0308, 0.0308, 0.0308, 0.03079, 0.03079, −0.03079, −0.03079, 0.03079, 0.03078, −0.03078, −0.03078, −0.03078, 0.03078, 0.03077, −0.03077, −0.03076, 0.03076, −0.03076, 0.03075, 0.03075, 0.03075, −0.03075, 0.03074, 0.03074, −0.03073, −0.03073, −0.03072, 0.03072, 0.03072, −0.03072, 0.03071, −0.0307, −0.0307, 0.0307, 0.03069, −0.03069, −0.03068, −0.03067, −0.03067, 0.03066, −0.03066, −0.03066, −0.03065, 0.03065, −0.03065, 0.03065, −0.03065, −0.03064, −0.03063, 0.03062, −0.03062, 0.03061, −0.03061, −0.0306, 0.0306, −0.0306, −0.03058, 0.03058, 0.03058, 0.03058, −0.03058, −0.03058, 0.03057, −0.03056, 0.03056, 0.03055, −0.03055, 0.03055, 0.03055, 0.03055, 0.03055, −0.03055, −0.03055, 0.03055, −0.03054, −0.03053, 0.03053, −0.03052, −0.03052, −0.03052, 0.03052, −0.03051, 0.03051, 0.0305, 0.0305, −0.0305, 0.03049, −0.03049, −0.03049, −0.03049, −0.03048, 0.03048, 0.03048, −0.03047, 0.03047, 0.03047, −0.03047, 0.03046, −0.03045, 0.03045, −0.03045, −0.03045, 0.03045, 0.03044, −0.03044, −0.03044, −0.03043, 0.03042, 0.03042, −0.03041, −0.0304, 0.0304, −0.03039, −0.03039, 0.03039, −0.03039, −0.03039, −0.03039, 0.03039, 0.03038, −0.03038, 0.03038, −0.03038, −0.03038, 0.03038, 0.03037, −0.03037, −0.03037, 0.03037, 0.03036, −0.03036, −0.03036, 0.03036, −0.03036, −0.03035, −0.03035, −0.03035, −0.03035, −0.03035, −0.03035, −0.03034, −0.03034, 0.03033, −0.03033, −0.03033, 0.03032, −0.03032, −0.03031, −0.03031, 0.03031, −0.03031, 0.03031, 0.03029, −0.03028, 0.03027, −0.03027, −0.03027, −0.03027, 0.03026, −0.03026, 0.03026, 0.03026, −0.03026, −0.03024, 0.03024, −0.03024, −0.03023, 0.03021, 0.03021, −0.0302, 0.0302, 0.03019, −0.03019, −0.03019, 0.03018, 0.03017, 0.03017, −0.03017, −0.03016, −0.03016, 0.03015, 0.03015, 0.03015, 0.03014, −0.03014, 0.03013, 0.03013, −0.03012, 0.03012, −0.03011, −0.03011, −0.03011, 0.03011, 0.0301, −0.0301, 0.0301, −0.03009, −0.03009, −0.03009, 0.03009, −0.03009, −0.03009, 0.03009, −0.03008, −0.03008, −0.03007, 0.03007, 0.03006, −0.03005, −0.03005, −0.03005, −0.03004, −0.03004, 0.03003, −0.03003, −0.03003, −0.03002, −0.03002, −0.03002, −0.03001, −0.03001, 0.03, −0.03, −0.02999, 0.02999, −0.02998, −0.02998, 0.02998, 0.02998, −0.02997, 0.02997, −0.02996, 0.02995, −0.02995, −0.02994, −0.02994, −0.02994, −0.02994, −0.02993, −0.02993, −0.02993, 0.02993, −0.02993, −0.02992, −0.02992, 0.02992, −0.02992, 0.02992, −0.0299, −0.0299, −0.0299, −0.0299, 0.0299, −0.02989, 0.02989, −0.02989, −0.02988, −0.02988, 0.02987, −0.02987, −0.02986, −0.02986, −0.02985, −0.02984, 0.02984, −0.02983, −0.02981, −0.02981, −0.02981, 0.02981, −0.02981, −0.0298, 0.0298, −0.0298, 0.0298, −0.0298, 0.02979, −0.02979, −0.02978, −0.02978, −0.02978, −0.02977, −0.02977, 0.02977, −0.02977, −0.02976, 0.02976, 0.02976, −0.02975, 0.02975, 0.02975, −0.02975, 0.02975, −0.02975, −0.02974, 0.02973, 0.02973, 0.02972, 0.02972, −0.02971, 0.02971, 0.02971, −0.0297, 0.0297, −0.02969, 0.02969, 0.02969, 0.02969, 0.02969, 0.02968, 0.02968, 0.02968, −0.02967, 0.02967, −0.02967, 0.02966, −0.02966, −0.02966, −0.02966, 0.02965, −0.02965, −0.02965, −0.02965, −0.02964, −0.02963, 0.02962, −0.02962, −0.02962, 0.02961, 0.02961, 0.02961, 0.0296, −0.0296, 0.0296, −0.02959, 0.02959, −0.02959, 0.02959, −0.02958, −0.02958, −0.02958, −0.02957, −0.02957, −0.02957, 0.02956, 0.02956, 0.02955, 0.02955, 0.02954, −0.02954, −0.02954, −0.02954, −0.02954, 0.02953, 0.02953, −0.02953, −0.02953, −0.02953, −0.02952, −0.02952, −0.0295, 0.0295, 0.0295, −0.0295, −0.0295, 0.0295, −0.0295, −0.02949, −0.02949, −0.02948, −0.02948, −0.02948, 0.02948, −0.02947, 0.02947, −0.02946, 0.02946, 0.02946, 0.02945, −0.02945, 0.02945, −0.02944, 0.02944, 0.02944, −0.02944, −0.02944, 0.02944, 0.02943, 0.02943, −0.02943, −0.02943, −0.02943, −0.02942, −0.02942, −0.02941, −0.0294, −0.0294, −0.0294, 0.0294, 0.0294, −0.02939, 0.02939, −0.02939, 0.02939, 0.02939, −0.02938, −0.02938, 0.02938, 0.02938, 0.02937, 0.02936, 0.02936, −0.02936, 0.02935, 0.02935, −0.02935, 0.02934, −0.02934, −0.02934, 0.02934, 0.02934, −0.02933, 0.02933, −0.02933, 0.02933, −0.02932, 0.02932, −0.02932, 0.02932, −0.02932, −0.02932, −0.02931, 0.02931, −0.0293, 0.0293, 0.0293, 0.0293, 0.0293, −0.02929, 0.02929, 0.02929, 0.02928, 0.02927, −0.02927, 0.02927, 0.02926, −0.02926, 0.02926, −0.02926, −0.02926, −0.02925, 0.02925, −0.02925, 0.02924, −0.02924, 0.02924, −0.02923, −0.02923, −0.02923, 0.02923, −0.02923, 0.02922, −0.02922, −0.02922, 0.02922, −0.02922, 0.02921, 0.02921, −0.02921, 0.0292, 0.0292, 0.0292, −0.02919, 0.02919, −0.02919, −0.02918, 0.02917, −0.02917, 0.02917, −0.02917, −0.02917, −0.02916, −0.02916, −0.02916, 0.02916, 0.02915, −0.02915, 0.02915, −0.02915, −0.02914, −0.02914, −0.02914, −0.02914, 0.02913, 0.02913, 0.02913, −0.02912, −0.02912, −0.02912, 0.02912, 0.02912, −0.02912, −0.02912, 0.02911, 0.02911, 0.02911, −0.02911, 0.0291, −0.02909, 0.02909, 0.02908, 0.02908, −0.02908, 0.02907, −0.02907, 0.02907, −0.02907, 0.02906, −0.02906, 0.02905, −0.02905, −0.02905, −0.02905, −0.02905, −0.02905, −0.02904, −0.02904, −0.02903, −0.02903, −0.02903, −0.02903, −0.02903, 0.02902, 0.02902, −0.02901, −0.02901, 0.02901, 0.029, 0.02899, −0.02899, −0.02899, 0.02899, −0.02898, 0.02898, −0.02898, 0.02898, −0.02898, −0.02897, −0.02897, −0.02897, 0.02896, 0.02896, −0.02896, 0.02895, 0.02895, −0.02895, −0.02895, 0.02895, −0.02894, −0.02894, 0.02894, 0.02893, −0.02893, 0.02892, −0.02892, 0.02892, −0.02892, 0.02892, −0.02891, −0.0289, −0.0289, 0.02889, −0.02889, −0.02889, −0.02889, 0.02888, −0.02888, −0.02888, −0.02888, −0.02886, 0.02886, 0.02885, −0.02885, 0.02885, 0.02884, 0.02883, −0.02883, −0.02883, −0.02883, −0.02883, 0.02883, −0.02882, −0.02882, −0.02882, 0.02881, 0.02881, 0.02881, 0.0288, −0.02879, 0.02879, −0.02878, −0.02878, −0.02878, −0.02878, −0.02878, 0.02878, 0.02877, 0.02877, 0.02877, −0.02877, −0.02876, 0.02876, 0.02875, −0.02875, 0.02875, 0.02874, −0.02874, −0.02874, 0.02873, −0.02873, 0.02873, −0.02873, −0.02873, −0.02872, −0.02872, −0.02871, −0.0287, −0.0287, −0.0287, −0.0287, 0.02869, −0.02868, 0.02868, 0.02867, −0.02867, 0.02867, 0.02867, −0.02867, 0.02866, −0.02866, 0.02866, −0.02866, −0.02865, −0.02865, −0.02865, 0.02865, 0.02865, −0.02864, 0.02864, 0.02864, 0.02863, 0.02863, −0.02863, −0.02862, −0.02862, 0.02861, 0.02861, −0.0286, 0.0286, 0.0286, 0.02859, 0.02859, −0.02859, 0.02858, −0.02858, −0.02858, 0.02858, −0.02857, −0.02857, −0.02857, 0.02856, 0.02856, −0.02855, 0.02855, 0.02855, −0.02855, 0.02854, −0.02854, 0.02854, 0.02853, 0.02853, −0.02853, 0.02853, 0.02852, −0.02852, 0.02852, 0.02851, 0.02851, −0.0285, −0.0285, −0.0285, 0.02849, −0.02848, −0.02848, 0.02848, 0.02847, 0.02847, −0.02847, −0.02847, −0.02847, 0.02846, −0.02846, 0.02846, −0.02845, 0.02845, −0.02845, 0.02845, 0.02845, 0.02845, −0.02844, 0.02844, −0.02844, −0.02843, 0.02843, 0.02843, 0.02843, −0.02842, −0.02842, −0.02842, −0.02842, 0.02841, −0.0284, −0.02839, 0.02838, −0.02838, −0.02837, 0.02837, 0.02837, 0.02837, −0.02836, 0.02835, −0.02835, −0.02835, 0.02835, −0.02835, 0.02834, 0.02834, 0.02834, 0.02833, −0.02833, 0.02833, 0.02833, 0.02832, 0.02832, −0.02831, 0.02831, 0.02831, −0.0283, −0.0283, −0.0283, −0.0283, −0.02829, −0.02828, 0.02828, 0.02827, −0.02827, 0.02826, −0.02826, −0.02825, −0.02824, 0.02824, −0.02824, 0.02823, −0.02823, 0.02823, −0.02822, −0.02822, 0.02822, −0.02822, −0.02822, 0.02821, 0.02821, 0.0282, −0.0282, 0.0282, −0.0282, 0.02819, −0.02819, −0.02819, 0.02819, 0.02819, 0.02819, −0.02818, −0.02818, 0.02818, −0.02818, −0.02818, −0.02818, 0.02816, 0.02816, −0.02816, −0.02816, 0.02816, −0.02816, −0.02815, −0.02815, −0.02815, 0.02815, −0.02815, −0.02814, 0.02814, −0.02813, 0.02812, −0.02812, −0.02811, −0.02811, −0.02811, −0.02811, −0.02811, 0.0281, 0.0281, −0.0281, −0.02809, 0.02808, −0.02808, −0.02808, 0.02808, 0.02807, −0.02807, 0.02807, −0.02806, −0.02806, −0.02806, −0.02806, −0.02806, −0.02805, −0.02805, −0.02805, 0.02805, −0.02805, 0.02804, 0.02804, −0.02804, 0.02803, 0.02803, −0.02802, −0.02802, −0.02802, −0.02802, −0.02802, −0.02802, 0.02801, −0.028, −0.028, 0.028, −0.02799, 0.02799, 0.02798, −0.02798, −0.02798, 0.02796, 0.02796, 0.02796, 0.02795, 0.02794, −0.02794, −0.02793, −0.02793, 0.02792, −0.02792, −0.02792, 0.02791, −0.02791, 0.0279, 0.0279, 0.0279, −0.0279, 0.02789, −0.02789, −0.02789, 0.02789, 0.02789, −0.02788, −0.02788, 0.02788, 0.02787, 0.02787, −0.02787, −0.02786, −0.02786, 0.02785, 0.02785, 0.02785, −0.02785, 0.02785, −0.02785, −0.02784, 0.02784, 0.02784, −0.02784, 0.02783, 0.02783, 0.02783, −0.02783, 0.02783, −0.02782, −0.02782, −0.02782, 0.02782, 0.02781, −0.02781, 0.0278, −0.0278, 0.0278, 0.02779, 0.02779, 0.02779, −0.02779, 0.02779, −0.02779, −0.02779, −0.02779, 0.02779, 0.02779, −0.02779, 0.02778, 0.02778, −0.02777, −0.02777, 0.02777, −0.02777, −0.02776, −0.02776, −0.02776, 0.02776, −0.02775, 0.02775, 0.02774, 0.02774, 0.02773, 0.02773, −0.02772, 0.02772, −0.02771, 0.02771, −0.02771, −0.0277, −0.02769, 0.02769, −0.02769, 0.02768, 0.02768, 0.02768, 0.02768, −0.02768, 0.02768, −0.02767, 0.02767, 0.02767, −0.02766, −0.02766, 0.02766, 0.02766, −0.02766, 0.02766, 0.02766, 0.02765, 0.02764, −0.02764, −0.02763, 0.02763, −0.02763, 0.02762, −0.02762, −0.02762, 0.02762, 0.02761, 0.02761, −0.0276, 0.0276, −0.0276, −0.0276, 0.0276, 0.0276, 0.02759, 0.02759, −0.02759, −0.02759, 0.02758, 0.02758, −0.02758, 0.02758, 0.02757, −0.02757, −0.02757, −0.02756, −0.02755, 0.02754, −0.02754, 0.02754, 0.02754, −0.02753, 0.02753, −0.02752, −0.02752, −0.02751, 0.02751, −0.0275, 0.0275, 0.0275, −0.02749, 0.02749, −0.02749, 0.02749, 0.02749, −0.02748, 0.02748, 0.02747, −0.02747, −0.02747, −0.02747, 0.02746, 0.02746, −0.02745, −0.02745, −0.02745, −0.02745, −0.02744, 0.02744, 0.02743, −0.02742, −0.02741, 0.02741, 0.02741, −0.0274, 0.0274, −0.0274, −0.0274, 0.02739, 0.02739, 0.02739, −0.02739, −0.02739, 0.02739, −0.02738, −0.02738, 0.02738, 0.02737, −0.02736, −0.02736, −0.02736, 0.02736, −0.02736, 0.02736, 0.02735, 0.02735, 0.02735, −0.02734, −0.02734, −0.02734, −0.02733, −0.02732, −0.02732, −0.02731, 0.02731, 0.02731, 0.0273, −0.0273, 0.0273, −0.02729, −0.02729, −0.02729, 0.02729, −0.02728, −0.02727, −0.02726, 0.02726, 0.02725, −0.02725, −0.02725, −0.02725, −0.02725, 0.02725, 0.02724, 0.02724, −0.02724, −0.02724, 0.02723, −0.02723, 0.02722, −0.02721, 0.0272, −0.0272, −0.02719, 0.02719, 0.02719, 0.02719, −0.02718, −0.02718, 0.02717, −0.02717, −0.02716, −0.02716, −0.02715, −0.02715, 0.02715, 0.02714, −0.02714, 0.02714, 0.02713, −0.02713, −0.02713, 0.02712, −0.02712, 0.02712, −0.02712, 0.02711, −0.02711, −0.02711, 0.0271, −0.02709, −0.02709, −0.02708, 0.02708, −0.02707, −0.02707, 0.02707, 0.02706, 0.02706, −0.02706, −0.02705, 0.02705, 0.02705, −0.02705, −0.02704, 0.02704, 0.02704, −0.02703, 0.02703, 0.02703, −0.02703, 0.02702, −0.02702, 0.02702, 0.02701, −0.02701, −0.02701, −0.027, −0.027, 0.027, −0.027, 0.027, 0.027, 0.02699, 0.02699, −0.02699, −0.02698, 0.02698, 0.02698, 0.02698, 0.02698, 0.02697, 0.02696, −0.02696, 0.02695, 0.02695, −0.02695, −0.02695, −0.02695, 0.02695, 0.02694, −0.02694, −0.02693, −0.02693, 0.02693, 0.02693, 0.02693, 0.02693, 0.02693, −0.02693, 0.02692, 0.02692, 0.02692, 0.02692, 0.02692, 0.02691, 0.02691, 0.0269, −0.0269, 0.0269, −0.0269, 0.02689, 0.02689, 0.02689, −0.02689, −0.02689, −0.02688, −0.02688, 0.02687, −0.02687, 0.02687, 0.02687, −0.02686, 0.02686, −0.02685, −0.02685, 0.02685, −0.02684, 0.02684, −0.02684, 0.02684, 0.02683, 0.02683, 0.02683, 0.02683, 0.02683, 0.02682, 0.02682, 0.02682, 0.02682, 0.02682, −0.02682, −0.02682, −0.02681, −0.0268, −0.0268, 0.0268, −0.0268, 0.02679, −0.02679, 0.02679, 0.02679, −0.02679, −0.02679, −0.02678, 0.02678, 0.02678, −0.02678, −0.02678, −0.02678, 0.02677, −0.02677, −0.02677, 0.02676, −0.02676, −0.02676, −0.02676, −0.02676, −0.02674, −0.02674, −0.02674, −0.02673, 0.02673, −0.02672, 0.02671, 0.02671, 0.0267, 0.0267, −0.02669, −0.02669, −0.02669, −0.02669, 0.02668, −0.02668, 0.02667, −0.02667, −0.02666, 0.02666, −0.02665, −0.02665, 0.02665, −0.02665, 0.02665, −0.02664, −0.02664, 0.02664, 0.02663, −0.02662, 0.02662, 0.02662, 0.02662, −0.02662, −0.02661, 0.02661, −0.02661, 0.02661, 0.02661, 0.02661, −0.02661, −0.0266, −0.0266, −0.02659, 0.02659, −0.02659, 0.02659, −0.02659, −0.02659, −0.02658, −0.02658, 0.02658, 0.02658, −0.02658, −0.02657, 0.02657, 0.02656, −0.02656, −0.02656, 0.02656, 0.02656, −0.02656, −0.02655, 0.02655, −0.02655, −0.02655, −0.02654, 0.02654, −0.02654, −0.02654, −0.02653, 0.02651, −0.02651, 0.02651, −0.02651, 0.02651, −0.02651, 0.02651, 0.02651, 0.02651, 0.0265, 0.0265, 0.0265, −0.0265, 0.0265, 0.0265, 0.02649, 0.02649, −0.02649, −0.02648, −0.02648, −0.02648, −0.02647, 0.02647, −0.02647, 0.02646, −0.02646, −0.02645, 0.02645, 0.02644, 0.02644, −0.02644, 0.02644, 0.02643, 0.02643, 0.02643, −0.02643, 0.02642, −0.02642, 0.02642, 0.02641, −0.02639, −0.02639, −0.02639, 0.02639, 0.02638, −0.02638, 0.02637, −0.02637, −0.02637, 0.02637, 0.02637, 0.02636, −0.02636, 0.02635, −0.02635, −0.02633, −0.02633, 0.02633, 0.02632, 0.02632, 0.02632, −0.02632, 0.02632, −0.02631, −0.02631, −0.02631, −0.02631, 0.02631, −0.02631, −0.02631, 0.0263, −0.0263, −0.0263, 0.0263, −0.0263, 0.02629, 0.02629, −0.02629, −0.02629, 0.02628, −0.02628, 0.02627, −0.02627, 0.02626, −0.02626, −0.02626, 0.02625, 0.02625, −0.02625, −0.02623, −0.02622, −0.02622, −0.02622, −0.02622, 0.02622, −0.02622, −0.02621, 0.02621, −0.02621, −0.02621, −0.0262, −0.0262, −0.0262, −0.02619, −0.02619, 0.02618, −0.02618, −0.02617, 0.02617, 0.02617, −0.02616, −0.02615, −0.02615, 0.02615, 0.02615, −0.02615, 0.02614, −0.02614, −0.02613, −0.02613, 0.02613, 0.02613, −0.02613, 0.02612, −0.02612, 0.02612, −0.02612, 0.02612, −0.02612, 0.02611, −0.02611, 0.02611, 0.02611, −0.02611, −0.02611, −0.02611, −0.02611, −0.0261, 0.0261, −0.0261, 0.0261, −0.0261, 0.0261, 0.02609, −0.02607, 0.02607, 0.02607, 0.02607, 0.02607, 0.02607, 0.02606, −0.02606, −0.02606, −0.02606, −0.02606, 0.02605, −0.02605, 0.02605, 0.02604, −0.02603, 0.02603, −0.02602, 0.02602, 0.02602, −0.02602, −0.02601, 0.02601, −0.02601, 0.026, 0.02599, 0.02599, 0.02599, −0.02599, 0.02599, −0.02599, 0.02598, −0.02598, −0.02597, −0.02597, −0.02596, −0.02596, −0.02596, −0.02595, −0.02595, −0.02595, 0.02595, −0.02593, −0.02593, 0.02593, 0.02593, 0.02592, 0.02592, −0.02591, 0.02591, −0.02591, −0.02591, 0.0259, 0.0259, 0.0259, 0.0259, −0.0259, −0.0259, 0.0259, −0.0259, 0.02589, 0.02589, 0.02589, −0.02589, −0.02589, −0.02588, 0.02588, −0.02588, 0.02588, −0.02587, 0.02586, −0.02585, −0.02585, −0.02585, −0.02585, −0.02584, 0.02583, −0.02583, 0.02583, 0.02583, 0.02582, 0.02582, −0.02581, −0.02581, 0.02581, 0.02581, 0.0258, −0.0258, 0.0258, 0.0258, 0.0258, −0.02579, 0.02579, −0.02578, 0.02578, 0.02578, −0.02578, 0.02578, 0.02578, −0.02577, −0.02577, −0.02577, 0.02577, 0.02576, 0.02576, −0.02576, −0.02576, −0.02575, −0.02575, −0.02575, 0.02575, 0.02575, 0.02574, −0.02574, −0.02574, 0.02574, −0.02574, 0.02574, −0.02573, 0.02573, 0.02573, −0.02573, −0.02573, −0.02573, −0.02572, −0.02572, −0.02572, −0.02572, 0.02572, 0.02571, −0.0257, 0.0257, −0.02569, 0.02568, 0.02568, −0.02568, −0.02568, 0.02567, −0.02567, −0.02567, −0.02567, 0.02566, −0.02566, −0.02566, 0.02566, 0.02566, 0.02565, −0.02564, 0.02564, 0.02564, 0.02564, 0.02564, 0.02564, −0.02564, 0.02563, 0.02563, −0.02563, −0.02562, −0.02562, −0.02561, −0.02561, −0.02561, −0.0256, −0.0256, −0.0256, 0.0256, 0.02559, −0.02559, −0.02559, −0.02559, 0.02559, 0.02559, 0.02559, 0.02558, 0.02558, 0.02558, −0.02557, 0.02557, 0.02557, 0.02557, −0.02557, −0.02557, 0.02557, −0.02557, 0.02556, 0.02556, −0.02556, 0.02555, 0.02555, −0.02554, −0.02553, 0.02553, −0.02553, 0.02553, 0.02553, −0.02553, 0.02552, 0.02552, 0.02552, 0.02552, −0.02551, 0.02551, −0.02551, −0.02551, −0.02551, 0.02551, 0.02551, 0.02551, −0.0255, −0.0255, −0.0255, 0.0255, −0.02549, −0.02549, 0.02549, 0.02548, −0.02548, −0.02548, 0.02548, −0.02548, −0.02547, −0.02547, −0.02547, 0.02547, −0.02546, 0.02546, −0.02546, −0.02546, −0.02545, −0.02545, −0.02545, −0.02545, 0.02545, −0.02544, −0.02544, 0.02544, 0.02544, −0.02544, 0.02543, 0.02543, −0.02542, 0.02542, 0.02542, 0.02541, −0.02541, −0.02541, −0.0254, −0.0254, −0.0254, −0.0254, 0.02539, 0.02539, −0.02539, −0.02539, −0.02538, 0.02538, −0.02537, −0.02537, 0.02537, 0.02536, 0.02536, −0.02536, −0.02536, 0.02536, 0.02535, −0.02535, −0.02535, −0.02535, −0.02535, −0.02534, −0.02534, 0.02534, 0.02534, −0.02533, −0.02533, 0.02533, 0.02533, −0.02532, 0.02532, −0.02532, 0.02532, 0.02532, 0.02532, 0.02532, −0.02531, −0.02531, −0.0253, −0.0253, −0.0253, −0.02529, −0.02529, 0.02529, −0.02528, 0.02528, −0.02528, 0.02528, 0.02528, −0.02528, −0.02527, −0.02527, 0.02527, −0.02526, −0.02526, 0.02526, −0.02526, −0.02525, −0.02525, 0.02525, 0.02525, −0.02524, −0.02524, −0.02523, −0.02523, −0.02523, 0.02523, 0.02523, 0.02522, 0.02522, −0.02522, 0.02522, −0.02522, −0.02521, −0.02521, −0.02521, 0.02521, 0.0252, −0.0252, 0.0252, −0.0252, −0.0252, 0.02519, −0.02519, 0.02518, −0.02518, 0.02518, −0.02518, 0.02517, 0.02517, 0.02516, −0.02516, 0.02516, 0.02516, −0.02515, −0.02515, −0.02515, −0.02515, 0.02514, 0.02514, 0.02514, −0.02514, 0.02513, −0.02513, −0.02513, 0.02513, −0.02512, 0.02512, 0.02511, 0.02511, 0.02511, 0.02511, −0.02511, 0.02509, −0.02509, 0.02508, −0.02508, 0.02508, −0.02508, 0.02507, −0.02506, 0.02506, 0.02506, −0.02504, −0.02503, 0.02503, −0.02503, 0.02503, 0.02502, 0.02502, 0.02502, −0.025, 0.025, 0.025, 0.025, −0.02499, −0.02499, 0.02499, 0.02499, −0.02499, −0.02498, 0.02498, −0.02498, −0.02498, −0.02497, 0.02497, 0.02497, 0.02496, 0.02496, 0.02496, −0.02496, 0.02496, 0.02496, 0.02495, 0.02495, −0.02494, 0.02494, −0.02494, 0.02494, −0.02494, 0.02493, 0.02493, 0.02492, −0.02492, −0.02492, 0.02492, 0.02492, 0.02492, 0.02492, −0.02492, −0.02491, 0.02491, −0.02491, −0.02491, 0.0249, 0.0249, 0.0249, −0.0249, −0.0249, −0.0249, 0.0249, −0.02489, 0.02489, 0.02489, −0.02489, 0.02489, 0.02488, −0.02488, 0.02488, 0.02488, 0.02488, −0.02488, −0.02488, −0.02488, 0.02487, 0.02487, 0.02487, 0.02487, −0.02485, 0.02485, −0.02485, 0.02484, −0.02484, 0.02484, 0.02484, 0.02484, −0.02484, 0.02483, −0.02483, −0.02482, −0.02482, −0.02481, 0.02481, −0.02481, −0.0248, −0.0248, −0.0248, −0.0248, −0.0248, 0.0248, −0.0248, −0.0248, 0.02479, −0.02479, −0.02479, −0.02479, 0.02478, 0.02478, −0.02478, 0.02477, −0.02477, 0.02477, −0.02477, −0.02477, −0.02477, 0.02477, 0.02477, 0.02477, 0.02476, 0.02476, 0.02475, −0.02475, 0.02474, −0.02474, −0.02473, −0.02473, 0.02473, −0.02473, −0.02472, −0.02471, 0.0247, −0.0247, 0.0247, 0.02469, 0.02469, 0.02469, −0.02468, −0.02468, −0.02468, −0.02468, −0.02468, 0.02468, 0.02467, −0.02467, 0.02467, −0.02467, 0.02467, 0.02467, 0.02467, 0.02466, −0.02465, −0.02465, 0.02465, −0.02464, −0.02464, −0.02464, 0.02463, −0.02462, 0.02461, −0.02461, −0.02461, 0.02461, −0.02461, −0.02461, −0.02461, −0.02461, 0.0246, 0.02459, −0.02459, −0.02458, −0.02458, −0.02458, 0.02458, 0.02457, −0.02456, −0.02456, −0.02456, −0.02456, −0.02456, −0.02456, −0.02455, 0.02454, −0.02454, 0.02454, 0.02453, −0.02453, 0.02453, 0.02453, 0.02452, −0.02452, −0.02451, −0.02451, 0.0245, −0.0245, 0.0245, 0.02449, −0.02449, 0.02449, −0.02449, 0.02449, −0.02448, −0.02448, 0.02448, 0.02448, 0.02448, −0.02447, 0.02447, −0.02447, −0.02446, −0.02446, −0.02446, −0.02445, 0.02445, 0.02445, 0.02445, 0.02445, −0.02445, 0.02445, 0.02445, 0.02444, −0.02444, 0.02443, −0.02443, −0.02443, −0.02443, 0.02443, 0.02443, 0.02442, −0.02442, 0.02442, 0.02442, 0.02441, 0.02441, −0.02441, 0.02441, 0.0244, −0.0244, 0.02439, 0.02439, 0.02439, 0.02438, −0.02438, −0.02438, −0.02438, −0.02437, 0.02437, 0.02437, 0.02436, −0.02436, 0.02436, 0.02436, 0.02436, −0.02435, −0.02435, −0.02434, 0.02434, −0.02433, −0.02433, −0.02432, −0.02432, 0.02432, −0.02432, −0.02431, −0.02431, 0.02431, −0.02431, 0.0243, 0.0243, 0.0243, −0.0243, 0.0243, −0.02429, −0.02429, 0.02428, −0.02428, 0.02427, −0.02427, 0.02427, 0.02427, 0.02427, 0.02427, 0.02427, 0.02427, −0.02427, 0.02426, 0.02426, −0.02426, 0.02426, 0.02426, 0.02425, 0.02424, −0.02424, 0.02424, −0.02423, −0.02423, −0.02423, 0.02423, 0.02422, 0.02422, 0.02422, 0.02422, 0.02422, −0.02421, −0.02421, 0.0242, 0.0242, −0.0242, −0.02419, −0.02418, −0.02418, −0.02417, 0.02417, 0.02416, 0.02416, 0.02416, −0.02416, 0.02415, 0.02415, 0.02415, −0.02414, 0.02414, 0.02413, −0.02412, −0.02412, −0.02412, 0.02412, 0.02412, −0.02412, −0.02412, −0.02411, −0.02411, 0.02411, 0.02411, −0.0241, −0.0241, −0.0241, −0.02409, 0.02409, −0.02409, 0.02409, −0.02408, −0.02408, −0.02408, −0.02408, 0.02408, 0.02408, −0.02407, −0.02407, −0.02407, −0.02406, −0.02405, 0.02405, −0.02405, 0.02405, 0.02405, −0.02405, −0.02404, −0.02404, 0.02404, −0.02404, 0.02404, −0.02404, 0.02403, 0.02403, −0.02403, 0.02403, 0.02402, 0.02402, 0.02402, −0.02402, −0.02402, 0.02401, 0.02401, −0.02401, −0.024, −0.024, −0.024, −0.024, 0.02399, 0.02398, −0.02398, −0.02398, −0.02397, −0.02397, −0.02397, 0.02396, 0.02396, 0.02396, −0.02396, 0.02396, 0.02395, 0.02395, −0.02395, 0.02395, 0.02394, −0.02394, 0.02393, −0.02393, −0.02392, 0.02392, −0.02392, −0.02392, −0.02391, −0.02391, −0.02391, −0.0239, 0.0239, −0.02389, −0.02389, −0.02389, 0.02389, 0.02387, 0.02387, 0.02387, 0.02386, −0.02386, −0.02386, 0.02386, 0.02385, −0.02385, 0.02384, −0.02384, −0.02383, 0.02383, −0.02383, 0.02382, −0.02381, −0.02381, −0.0238, 0.0238, −0.0238, 0.0238, −0.0238, 0.0238, 0.02379, −0.02379, −0.02379, −0.02379, 0.02379, 0.02379, −0.02378, −0.02378, 0.02378, 0.02377, 0.02376, −0.02376, −0.02376, 0.02375, 0.02375, −0.02375, −0.02373, −0.02373, 0.02373, −0.02373, −0.02372, 0.02372, −0.02372, 0.02372, −0.02371, 0.02371, 0.02371, 0.02371, 0.02371, −0.0237, 0.0237, −0.0237, −0.0237, 0.02369, 0.02369, 0.02369, −0.02369, 0.02369, 0.02368, 0.02368, −0.02367, −0.02367, 0.02366, −0.02366, −0.02366, 0.02366, 0.02366, 0.02366, 0.02365, −0.02365, −0.02365, −0.02365, 0.02365, −0.02364, −0.02364, −0.02364, 0.02364, 0.02364, −0.02364, 0.02364, 0.02363, −0.02363, −0.02363, −0.02362, 0.02362, −0.02362, 0.02362, −0.02361, 0.02361, 0.02361, 0.0236, 0.0236, 0.0236, −0.0236, 0.0236, −0.0236, 0.0236, −0.0236, −0.0236, 0.02359, 0.02358, −0.02358, 0.02358, −0.02358, −0.02358, −0.02358, 0.02358, −0.02358, 0.02357, −0.02357, −0.02357, 0.02356, −0.02356, −0.02356, 0.02356, 0.02356, −0.02355, 0.02355, 0.02355, −0.02355, 0.02354, 0.02354, 0.02354, −0.02353, 0.02353, −0.02353, −0.02353, 0.02352, −0.02352, 0.02351, 0.02351, 0.0235, 0.0235, −0.02349, −0.02349, 0.02349, −0.02349, −0.02348, 0.02348, 0.02348, −0.02348, −0.02347, 0.02347, 0.02346, −0.02346, 0.02346, 0.02346, −0.02346, −0.02345, −0.02345, 0.02345, −0.02345, −0.02345, −0.02345, 0.02345, −0.02345, 0.02344, −0.02344, 0.02344, 0.02344, 0.02343, 0.02343, −0.02343, −0.02343, −0.02343, 0.02342, −0.02342, 0.02342, 0.02341, −0.02341, 0.0234, −0.0234, −0.0234, 0.02339, 0.02339, −0.02339, −0.02339, −0.02338, −0.02338, −0.02338, −0.02338, 0.02338, 0.02337, −0.02337, 0.02337, −0.02336, 0.02336, −0.02336, 0.02336, −0.02336, 0.02336, 0.02335, −0.02335, −0.02334, −0.02333, 0.02333, −0.02333, 0.02333, 0.02332, 0.02331, −0.02331, −0.02331, −0.02331, −0.02331, 0.0233, −0.0233, −0.0233, −0.0233, 0.0233, 0.0233, 0.02329, 0.02329, −0.02329, −0.02329, 0.02329, 0.02329, 0.02328, −0.02328, −0.02328, −0.02328, −0.02328, 0.02327, −0.02327, 0.02327, 0.02327, −0.02326, −0.02326, −0.02326, 0.02325, −0.02325, −0.02325, 0.02324, 0.02324, 0.02324, 0.02324, −0.02324, 0.02323, −0.02323, 0.02323, 0.02322, 0.02322, 0.02322, −0.02321, 0.02321, −0.02321, −0.0232, 0.0232, 0.02319, 0.02319, −0.02319, 0.02318, −0.02318, 0.02318, −0.02318, −0.02318, −0.02317, 0.02316, −0.02316, −0.02316, 0.02316, 0.02315, 0.02315, −0.02315, −0.02314, −0.02314, 0.02313, −0.02313, 0.02313, −0.02313, −0.02313, −0.02312, −0.02311, −0.02311, −0.02311, 0.02311, −0.0231, 0.0231, −0.0231, 0.0231, −0.0231, 0.0231, −0.02308, 0.02308, −0.02308, −0.02308, −0.02308, −0.02307, 0.02307, −0.02307, −0.02307, −0.02306, 0.02306, −0.02306, 0.02306, −0.02306, 0.02306, 0.02305, 0.02305, −0.02305, −0.02305, −0.02305, −0.02304, −0.02304, −0.02304, −0.02304, −0.02304, 0.02304, 0.02303, 0.02303, −0.02303, −0.02302, −0.02302, −0.02301, −0.02301, 0.023, −0.023, −0.023, 0.02299, −0.02298, 0.02298, 0.02298, −0.02298, −0.02298, −0.02298, −0.02298, −0.02297, 0.02297, −0.02297, −0.02297, 0.02297, 0.02296, −0.02296, 0.02295, 0.02295, 0.02295, −0.02295, 0.02295, −0.02294, 0.02294, 0.02294, 0.02294, −0.02294, 0.02294, 0.02293, −0.02293, 0.02293, 0.02293, −0.02293, −0.02293, 0.02292, 0.02292, 0.02292, −0.02292, −0.02292, −0.02291, 0.02291, −0.02291, 0.02291, −0.02291, 0.0229, 0.0229, −0.02289, 0.02289, −0.02289, −0.02288, 0.02288, −0.02288, 0.02287, −0.02287, −0.02287, −0.02287, 0.02287, 0.02287, 0.02286, −0.02286, −0.02286, −0.02285, −0.02285, −0.02285, −0.02285, 0.02285, −0.02284, 0.02284, 0.02284, 0.02284, 0.02284, −0.02283, 0.02283, −0.02283, 0.02283, 0.02282, −0.02282, 0.02282, 0.02281, −0.02281, −0.0228, −0.0228, 0.0228, 0.0228, 0.02279, 0.02279, 0.02279, 0.02279, 0.02278, 0.02278, −0.02277, −0.02277, 0.02277, 0.02276, 0.02276, 0.02276, 0.02276, 0.02276, 0.02275, −0.02275, 0.02275, −0.02274, 0.02274, 0.02274, 0.02274, 0.02274, 0.02273, −0.02273, −0.02273, 0.02273, −0.02272, −0.02272, 0.02271, 0.02271, 0.02271, −0.02271, 0.02271, −0.02271, −0.0227, −0.0227, 0.02269, 0.02269, −0.02269, −0.02269, 0.02268, 0.02268, −0.02268, 0.02268, 0.02267, −0.02267, −0.02267, −0.02267, −0.02266, −0.02266, −0.02266, −0.02266, 0.02265, −0.02265, 0.02265, −0.02264, 0.02264, −0.02263, −0.02263, 0.02263, −0.02263, 0.02262, 0.02262, −0.02262, 0.02262, 0.02262, 0.02262, 0.02262, 0.02261, 0.02261, −0.02261, −0.02261, −0.02261, 0.02261, 0.0226, 0.0226, −0.02259, 0.02259, −0.02258, 0.02258, 0.02258, 0.02258, −0.02258, 0.02258, −0.02258, −0.02257, 0.02257, 0.02257, 0.02256, 0.02256, −0.02256, −0.02255, −0.02255, 0.02254, 0.02254, 0.02254, 0.02253, −0.02253, −0.02252, −0.02252, 0.02252, −0.02252, 0.02252, 0.02251, −0.02251, −0.02251, −0.02251, 0.02251, −0.02251, −0.02251, 0.0225, 0.0225, 0.0225, −0.0225, −0.0225, −0.0225, 0.02248, −0.02248, 0.02248, 0.02247, −0.02247, −0.02247, −0.02246, 0.02246, 0.02246, −0.02245, 0.02245, −0.02245, 0.02244, −0.02244, 0.02244, −0.02243, 0.02243, −0.02243, 0.02243, 0.02243, 0.02243, −0.02242, −0.02242, −0.02242, −0.02241, 0.02241, −0.02241, 0.02241, 0.02241, 0.02241, 0.0224, 0.0224, 0.0224, 0.02239, 0.02239, 0.02239, −0.02239, 0.02239, 0.02239, 0.02238, −0.02238, 0.02238, 0.02238, 0.02238, −0.02238, −0.02237, −0.02237, 0.02237, 0.02237, −0.02237, 0.02236, −0.02236, 0.02236, −0.02236, 0.02235, 0.02235, −0.02235, 0.02235, 0.02234, 0.02233, 0.02233, −0.02233, −0.02232, 0.02232, −0.02232, 0.02232, −0.02232, 0.02231, 0.02231, −0.02231, −0.0223, 0.0223, −0.0223, −0.02229, −0.02229, −0.02229, −0.02229, 0.02229, 0.02229, 0.02229, −0.02229, 0.02229, −0.02229, −0.02229, −0.02228, 0.02228, 0.02228, 0.02227, 0.02227, −0.02227, −0.02227, −0.02226, −0.02226, 0.02226, 0.02226, 0.02225, 0.02225, −0.02225, −0.02225, 0.02225, 0.02225, −0.02225, 0.02224, −0.02224, 0.02224, −0.02224, −0.02224, −0.02224, 0.02223, −0.02223, 0.02223, 0.02223, 0.02223, −0.02222, 0.02222, −0.02222, 0.02222, 0.02221, 0.02221, 0.02221, 0.02221, 0.0222, 0.0222, −0.0222, −0.0222, 0.0222, 0.0222, 0.0222, −0.02219, −0.02219, −0.02219, −0.02219, 0.02219, 0.02219, −0.02219, −0.02218, 0.02218, 0.02218, 0.02218, −0.02217, −0.02217, −0.02217, 0.02217, 0.02217, −0.02216, −0.02216, −0.02216, −0.02216, 0.02216, −0.02215, −0.02215, 0.02215, 0.02215, −0.02214, 0.02214, −0.02214, −0.02213, −0.02213, 0.02213, 0.02213, 0.02213, 0.02213, 0.02212, 0.02212, −0.02212, 0.02212, 0.02211, 0.02211, 0.02211, −0.0221, 0.0221, −0.0221, 0.02209, −0.02209, −0.02209, −0.02209, 0.02209, −0.02208, 0.02208, 0.02208, 0.02207, 0.02207, −0.02207, 0.02207, 0.02206, 0.02206, 0.02206, −0.02206, 0.02206, −0.02206, −0.02206, 0.02205, 0.02205, −0.02205, 0.02205, −0.02204, 0.02204, −0.02204, −0.02204, 0.02203, −0.02203, 0.02203, 0.02203, 0.02202, 0.02202, −0.02202, −0.02202, −0.02202, 0.02202, 0.02201, 0.02201, −0.02201, 0.02201, −0.02201, 0.02201, 0.022, 0.022, 0.022, −0.022, 0.02199, −0.02199, 0.02199, 0.02199, 0.02198, −0.02198, 0.02197, −0.02197, 0.02197, 0.02196, −0.02196, 0.02196, 0.02196, −0.02196, −0.02195, 0.02195, 0.02195, 0.02195, 0.02194, 0.02194, −0.02194, 0.02194, −0.02193, −0.02193, 0.02193, 0.02193, −0.02193, 0.02193, 0.02192, 0.02192, 0.02191, −0.02191, 0.02191, −0.02191, −0.02191, −0.0219, −0.0219, −0.0219, 0.0219, 0.02189, −0.02189, 0.02189, −0.02188, −0.02188, −0.02188, −0.02187, −0.02187, −0.02187, 0.02186, −0.02186, −0.02186, −0.02186, 0.02186, 0.02186, −0.02186, −0.02186, 0.02186, 0.02185, −0.02185, 0.02185, −0.02184, 0.02184, −0.02184, 0.02184, 0.02183, 0.02183, 0.02183, −0.02183, −0.02183, −0.02182, 0.02182, 0.02182, −0.02182, 0.02182, 0.02181, −0.02181, 0.02181, 0.02181, −0.02181, −0.02181, 0.02181, 0.0218, −0.0218, 0.0218, 0.0218, −0.0218, −0.02179, −0.02179, 0.02179, −0.02178, −0.02178, −0.02178, 0.02178, 0.02177, −0.02177, 0.02177, 0.02177, −0.02177, 0.02176, −0.02176, 0.02176, 0.02176, −0.02176, −0.02176, 0.02176, −0.02175, 0.02175, 0.02175, −0.02175, 0.02175, 0.02175, −0.02175, 0.02174, −0.02174, 0.02174, −0.02173, −0.02173, 0.02173, −0.02173, 0.02173, −0.02172, 0.02172, 0.02172, 0.02172, 0.02172, 0.02171, 0.02171, 0.02171, 0.02171, −0.0217, 0.0217, 0.0217, −0.0217, −0.0217, −0.02169, 0.02169, −0.02169, 0.02169, −0.02169, 0.02168, 0.02167, 0.02167, −0.02167, 0.02167, −0.02166, 0.02166, 0.02166, −0.02165, −0.02165, −0.02165, 0.02165, 0.02165, 0.02165, 0.02164, −0.02164, −0.02164, −0.02164, −0.02164, 0.02164, −0.02164, −0.02163, 0.02163, 0.02163, −0.02163, 0.02163, 0.02163, 0.02163, −0.02162, 0.02162, 0.02161, 0.02161, 0.02161, 0.02161, 0.0216, 0.0216, −0.0216, 0.0216, 0.0216, 0.0216, 0.02159, 0.02159, 0.02159, 0.02159, 0.02159, −0.02159, 0.02158, 0.02158, −0.02158, −0.02158, 0.02158, 0.02157, −0.02156, −0.02156, 0.02156, −0.02156, 0.02156, −0.02156, −0.02156, 0.02156, −0.02155, 0.02155, 0.02155, −0.02155, −0.02154, 0.02154, −0.02154, −0.02153, −0.02153, −0.02153, −0.02153, −0.02153, −0.02152, 0.02152, −0.02151, −0.02151, −0.02151, −0.02151, 0.0215, −0.0215, −0.0215, −0.0215, 0.02149, −0.02149, −0.02149, 0.02149, −0.02149, 0.02148, 0.02148, −0.02147, −0.02147, 0.02146, 0.02146, 0.02145, 0.02145, −0.02145, 0.02144, −0.02144, −0.02144, 0.02144, −0.02144, 0.02143, 0.02143, 0.02143, 0.02143, 0.02143, −0.02143, 0.02142, −0.02142, 0.02142, 0.02142, 0.02142, 0.02142, 0.02142, −0.02141, −0.02141, −0.02141, 0.0214, 0.0214, 0.0214, −0.0214, −0.0214, −0.02139, 0.02139, −0.02139, 0.02138, 0.02138, 0.02137, 0.02136, −0.02136, 0.02136, −0.02135, −0.02135, 0.02135, −0.02135, −0.02135, 0.02135, −0.02135, −0.02134, 0.02134, 0.02134, −0.02133, 0.02133, 0.02133, 0.02133, 0.02132, −0.02132, −0.02132, −0.02132, 0.02132, 0.02132, −0.02132, −0.02132, 0.02131, −0.02131, −0.02131, −0.02131, 0.02131, −0.02131, −0.02131, 0.0213, 0.0213, 0.0213, 0.0213, 0.0213, 0.02129, 0.02129, −0.02129, −0.02129, 0.02129, 0.02128, −0.02128, 0.02128, 0.02128, 0.02128, 0.02127, 0.02126, −0.02126, 0.02125, −0.02125, −0.02124, −0.02124, 0.02123, −0.02123, −0.02123, −0.02123, −0.02123, 0.02122, 0.02122, −0.02122, 0.02122, −0.02122, 0.02121, 0.02121, 0.02121, −0.02121, 0.0212, −0.0212, 0.0212, −0.02119, −0.02119, 0.02119, −0.02119, 0.02118, 0.02118, 0.02118, 0.02118, −0.02118, 0.02118, 0.02117, −0.02117, 0.02117, 0.02117, 0.02117, −0.02116, −0.02116, 0.02116, −0.02116, −0.02115, 0.02115, −0.02115, 0.02115, −0.02115, −0.02115, 0.02114, −0.02114, 0.02114, 0.02113, −0.02112, 0.02112, −0.02112, 0.02112, 0.02112, −0.02112, 0.02111, −0.02111, 0.02111, 0.02111, −0.02111, 0.02111, −0.0211, 0.0211, −0.0211, −0.0211, 0.02109, −0.02108, 0.02108, −0.02108, −0.02107, 0.02107, 0.02107, −0.02107, 0.02107, 0.02107, 0.02107, −0.02107, 0.02106, 0.02106, −0.02105, −0.02105, −0.02105, −0.02105, −0.02105, −0.02105, 0.02104, 0.02104, 0.02104, −0.02103, −0.02103, 0.02103, 0.02103, −0.02102, −0.02102, 0.02102, −0.02102, 0.02102, −0.02102, −0.02101, −0.02101, 0.02101, −0.02101, 0.02101, −0.02101, −0.021, 0.021, 0.02099, −0.02099, −0.02099, 0.02098, 0.02098, −0.02098, 0.02097, −0.02097, 0.02097, −0.02096, −0.02096, −0.02096, 0.02096, 0.02096, 0.02095, −0.02095, −0.02095, 0.02095, −0.02095, −0.02095, 0.02095, 0.02094, −0.02094, −0.02094, −0.02094, 0.02094, −0.02094, −0.02093, −0.02093, −0.02093, −0.02093, −0.02093, −0.02093, 0.02093, −0.02092, 0.02092, −0.02092, 0.02092, 0.02092, −0.02091, 0.02091, −0.0209, −0.0209, 0.0209, 0.0209, −0.02089, 0.02089, 0.02089, 0.02089, −0.02089, 0.02089, −0.02088, −0.02088, 0.02088, −0.02088, −0.02087, 0.02087, 0.02087, 0.02086, −0.02086, −0.02086, 0.02085, −0.02085, 0.02085, −0.02085, −0.02084, −0.02084, −0.02084, −0.02084, −0.02084, −0.02084, −0.02083, 0.02083, 0.02083, −0.02083, 0.02083, −0.02082, 0.02082, 0.02082, 0.02082, 0.02082, −0.02082, 0.02081, −0.02081, 0.02081, 0.02081, 0.02081, 0.02081, 0.02081, 0.02081, −0.02081, 0.02081, −0.0208, −0.0208, 0.0208, 0.02079, −0.02079, 0.02079, −0.02079, 0.02079, −0.02078, −0.02078, −0.02078, −0.02078, 0.02078, 0.02078, −0.02078, 0.02078, 0.02077, 0.02077, 0.02077, 0.02077, −0.02077, −0.02077, −0.02077, −0.02076, 0.02076, 0.02076, −0.02076, −0.02075, 0.02075, −0.02075, −0.02075, −0.02075, 0.02074, −0.02074, 0.02074, 0.02073, −0.02073, 0.02073, 0.02073, 0.02073, −0.02073, 0.02073, −0.02073, 0.02072, 0.02072, 0.02071, −0.02071, 0.02071, 0.02071, 0.02071, 0.02071, 0.0207, 0.0207, 0.0207, 0.0207, −0.0207, 0.0207, −0.02069, 0.02069, −0.02068, 0.02068, 0.02068, −0.02068, −0.02068, 0.02068, 0.02068, 0.02067, −0.02067, 0.02067, 0.02067, −0.02067, 0.02066, −0.02065, −0.02065, −0.02065, −0.02065, −0.02065, 0.02065, 0.02064, −0.02064, 0.02064, 0.02064, 0.02063, −0.02063, −0.02063, 0.02063, −0.02063, −0.02062, 0.02062, 0.02062, −0.02062, −0.02062, 0.02062, 0.02062, −0.02061, 0.02061, −0.0206, −0.0206, 0.0206, 0.02059, −0.02058, −0.02058, −0.02058, −0.02058, −0.02058, 0.02058, 0.02057, 0.02057, −0.02056, −0.02056, −0.02056, 0.02056, 0.02056, −0.02055, −0.02055, −0.02055, −0.02054, −0.02054, 0.02054, 0.02054, −0.02054, −0.02053, −0.02053, 0.02053, −0.02053, −0.02053, 0.02053, −0.02053, −0.02052, 0.02052, −0.02052, 0.02052, 0.02052, −0.02052, −0.02052, −0.02052, 0.02052, 0.02051, 0.02051, 0.02051, −0.02051, −0.02051, 0.02051, −0.02051, −0.02051, 0.02051, −0.02051, −0.0205, 0.0205, −0.0205, −0.0205, 0.0205, 0.02049, 0.02049, 0.02049, 0.02049, 0.02049, −0.02048, −0.02048, 0.02048, 0.02048, 0.02047, −0.02047, 0.02047, −0.02045, −0.02045, −0.02045, 0.02044, −0.02044, 0.02044, 0.02044, −0.02043, 0.02043, 0.02043, −0.02043, 0.02043, −0.02043, 0.02042, 0.02042, 0.02042, −0.02041, −0.02041, −0.02041, −0.02041, 0.02041, 0.02041, −0.0204, 0.0204, −0.0204, −0.02039, 0.02039, 0.02039, −0.02039, −0.02039, 0.02039, 0.02039, 0.02038, −0.02038, −0.02038, 0.02038, −0.02038, 0.02038, −0.02038, 0.02037, −0.02037, 0.02037, 0.02037, −0.02037, −0.02037, 0.02037, 0.02036, 0.02036, −0.02036, 0.02036, 0.02036, 0.02036, 0.02035, −0.02035, 0.02035, −0.02035, 0.02035, 0.02035, −0.02035, 0.02034, 0.02033, 0.02033, 0.02032, 0.02032, −0.02031, 0.02031, 0.02031, −0.02031, 0.0203, 0.0203, −0.0203, −0.02029, −0.02029, 0.02029, 0.02029, 0.02029, 0.02029, 0.02029, 0.02029, −0.02028, −0.02028, −0.02028, 0.02028, −0.02028, 0.02027, 0.02027, −0.02027, −0.02027, −0.02026, −0.02026, 0.02026, −0.02025, −0.02025, −0.02025, −0.02025, −0.02025, 0.02025, −0.02024, 0.02024, −0.02024, −0.02024, 0.02024, 0.02024, 0.02024, 0.02024, 0.02024, −0.02024, 0.02023, 0.02023, −0.02023, 0.02023, −0.02022, −0.02022, −0.02022, −0.02022, 0.02021, 0.02021, −0.02021, 0.02021, 0.02021, 0.02021, −0.02021, 0.02021, 0.02021, −0.02021, −0.0202, 0.0202, −0.0202, 0.0202, 0.0202, 0.0202, 0.02019, 0.02019, 0.02018, −0.02018, −0.02018, 0.02017, −0.02016, 0.02015, −0.02015, −0.02015, 0.02015, −0.02015, −0.02014, 0.02014, 0.02014, −0.02014, −0.02014, −0.02014, −0.02013, −0.02013, 0.02013, 0.02013, 0.02012, −0.02012, −0.02012, −0.02012, 0.02012, −0.02012, −0.02011, −0.02011, −0.02011, 0.0201, 0.0201, 0.0201, −0.02009, 0.02009, 0.02009, −0.02009, 0.02009, 0.02008, −0.02008, −0.02007, −0.02007, 0.02007, 0.02007, 0.02006, −0.02006, 0.02006, 0.02006, 0.02006, 0.02006, 0.02005, −0.02005, 0.02005, −0.02004, −0.02004, −0.02004, −0.02004, 0.02003, 0.02003, −0.02003, 0.02003, −0.02003, 0.02003, −0.02002, −0.02002, −0.02002, −0.02002, −0.02001, 0.02001, 0.02001, 0.02001, 0.02001, −0.02, −0.02, −0.02, 0.02, 0.02, −0.02, −0.01999, 0.01999, −0.01999, −0.01999, 0.01998, 0.01998, −0.01998, −0.01998, −0.01998, 0.01997, −0.01997, 0.01997, 0.01996, −0.01996, 0.01996, 0.01995, 0.01995, −0.01995, 0.01995, −0.01995, −0.01995, −0.01994, 0.01994, −0.01994, −0.01993, 0.01993, −0.01993, 0.01993, −0.01992, 0.01992, −0.01992, −0.01992, −0.01991, 0.01991, 0.0199, −0.01989, 0.01989, 0.01989, 0.01988, 0.01988, −0.01988, −0.01988, −0.01988, 0.01988, −0.01987, −0.01987, −0.01987, −0.01986, 0.01986, −0.01986, −0.01986, −0.01986, 0.01986, 0.01986, 0.01985, −0.01985, −0.01985, 0.01984, 0.01984, −0.01984, 0.01984, −0.01983, 0.01983, −0.01983, 0.01983, −0.01983, −0.01982, 0.01982, 0.01981, 0.01981, −0.01981, −0.01981, −0.01981, −0.01981, −0.0198, 0.0198, −0.0198, 0.0198, 0.0198, 0.0198, 0.01979, 0.01979, −0.01979, −0.01979, −0.01979, −0.01978, −0.01978, −0.01978, 0.01978, −0.01978, 0.01978, −0.01978, 0.01978, 0.01978, 0.01978, −0.01977, 0.01977, 0.01977, −0.01977, 0.01977, 0.01977, −0.01976, −0.01976, −0.01976, 0.01976, 0.01976, −0.01975, 0.01975, −0.01974, 0.01974, −0.01974, 0.01974, 0.01973, 0.01973, −0.01973, −0.01972, −0.01972, 0.01972, 0.01972, −0.01972, −0.01972, 0.01971, 0.01971, 0.01971, 0.01971, 0.01971, −0.0197, −0.0197, 0.0197, 0.01968, −0.01968, 0.01968, −0.01968, −0.01968, 0.01967, 0.01967, −0.01966, 0.01966, −0.01966, −0.01966, −0.01966, 0.01964, 0.01964, −0.01964, 0.01964, 0.01963, −0.01963, 0.01963, 0.01963, 0.01962, 0.01962, 0.01962, 0.01962, 0.01962, 0.01962, −0.01962, −0.01961, −0.0196, −0.0196, 0.0196, −0.0196, −0.01959, 0.01959, −0.01959, −0.01959, −0.01959, −0.01959, −0.01959, 0.01959, −0.01958, 0.01957, 0.01957, −0.01957, −0.01956, −0.01956, 0.01956, −0.01956, 0.01956, 0.01956, 0.01955, −0.01955, 0.01955, −0.01955, −0.01955, −0.01955, −0.01955, −0.01955, −0.01955, −0.01954, −0.01953, −0.01953, −0.01953, 0.01952, 0.01952, 0.01951, −0.0195, 0.0195, −0.0195, −0.0195, −0.0195, 0.0195, −0.0195, 0.0195, −0.01949, 0.01949, −0.01949, 0.01949, 0.01949, 0.01949, 0.01948, −0.01948, 0.01948, 0.01948, 0.01947, 0.01947, 0.01947, 0.01947, −0.01947, 0.01947, 0.01947, 0.01946, 0.01946, −0.01946, −0.01945, −0.01945, −0.01945, −0.01945, 0.01944, −0.01944, −0.01944, −0.01943, 0.01943, 0.01943, 0.01943, 0.01943, −0.01942, 0.01942, −0.01942, 0.01942, −0.01942, −0.01942, 0.01941, 0.01941, 0.01941, 0.01941, 0.0194, 0.0194, 0.0194, −0.01939, 0.01939, −0.01939, 0.01939, −0.01939, 0.01939, 0.01939, 0.01938, −0.01938, −0.01937, −0.01937, 0.01937, 0.01937, 0.01937, −0.01937, 0.01937, 0.01937, −0.01937, 0.01936, −0.01936, 0.01936, 0.01936, −0.01936, −0.01936, 0.01936, 0.01936, 0.01936, −0.01935, 0.01935, 0.01935, 0.01935, 0.01935, 0.01935, −0.01935, 0.01934, −0.01934, 0.01934, 0.01934, −0.01934, −0.01934, 0.01934, 0.01933, 0.01933, −0.01933, 0.01933, −0.01933, −0.01932, −0.01932, −0.01932, −0.01931, 0.01931, −0.01931, −0.01931, 0.01931, 0.0193, −0.0193, 0.0193, −0.0193, 0.0193, 0.0193, −0.0193, −0.0193, −0.0193, 0.01929, −0.01929, −0.01929, 0.01929, −0.01928, −0.01928, 0.01928, 0.01928, −0.01927, −0.01927, −0.01927, −0.01927, 0.01926, 0.01926, −0.01926, 0.01926, 0.01926, −0.01926, 0.01925, −0.01925, 0.01925, −0.01925, −0.01925, 0.01925, −0.01924, 0.01924, −0.01924, 0.01924, 0.01924, 0.01924, 0.01923, −0.01923, 0.01923, −0.01922, 0.01922, −0.01922, 0.01921, 0.01921, 0.01921, 0.01921, 0.0192, −0.0192, 0.0192, 0.0192, 0.01919, −0.01919, −0.01919, 0.01919, −0.01919, −0.01919, 0.01918, 0.01918, −0.01918, −0.01918, −0.01917, −0.01917, 0.01916, −0.01915, 0.01915, −0.01915, 0.01915, −0.01915, 0.01915, −0.01915, −0.01914, 0.01914, 0.01914, 0.01914, 0.01914, 0.01914, −0.01914, −0.01913, 0.01913, −0.01913, 0.01913, 0.01913, 0.01913, −0.01913, −0.01912, −0.01912, 0.01912, −0.01912, −0.01912, −0.01912, 0.01912, −0.01911, −0.01911, −0.01911, −0.01911, −0.01911, 0.0191, −0.0191, −0.0191, −0.0191, −0.0191, 0.0191, 0.01909, 0.01909, −0.01909, −0.01909, 0.01909, −0.01909, −0.01907, 0.01907, −0.01906, −0.01906, 0.01906, −0.01905, 0.01905, 0.01905, 0.01904, 0.01904, 0.01904, 0.01904, −0.01903, −0.01903, −0.01903, −0.01903, 0.01903, 0.01903, −0.01903, 0.01903, −0.01903, −0.01902, 0.01902, 0.01902, −0.01902, −0.01902, −0.01902, 0.01902, 0.01901, −0.01901, 0.01901, 0.01901, −0.01901, 0.01901, −0.019, −0.019, −0.019, −0.019, 0.019, −0.019, 0.019, 0.019, 0.019, −0.01899, 0.01899, −0.01899, −0.01899, 0.01898, 0.01898, 0.01898, 0.01898, −0.01897, −0.01897, −0.01897, −0.01897, 0.01897, −0.01897, 0.01897, 0.01897, 0.01896, 0.01896, −0.01895, −0.01895, 0.01895, 0.01895, 0.01895, −0.01895, 0.01895, 0.01895, 0.01894, 0.01894, 0.01894, 0.01894, 0.01893, −0.01893, 0.01893, −0.01893, −0.01892, −0.01892, −0.01892, −0.01892, 0.01892, 0.01892, −0.01891, 0.01891, 0.0189, 0.0189, 0.0189, 0.0189, −0.0189, −0.01889, 0.01889, −0.01889, 0.01889, −0.01889, −0.01888, 0.01888, −0.01888, −0.01888, 0.01887, −0.01887, −0.01887, 0.01887, 0.01887, 0.01887, 0.01886, −0.01886, −0.01886, −0.01886, −0.01886, 0.01885, −0.01885, −0.01885, 0.01885, 0.01884, 0.01884, −0.01884, −0.01883, 0.01883, 0.01883, 0.01882, 0.01882, −0.01882, −0.01882, −0.01882, −0.01882, −0.01882, 0.01881, −0.01881, 0.01881, 0.01881, −0.0188, 0.0188, 0.0188, 0.0188, 0.0188, 0.0188, −0.0188, −0.01879, −0.01879, −0.01879, −0.01879, −0.01879, −0.01879, 0.01879, 0.01878, 0.01878, 0.01878, −0.01878, −0.01877, −0.01877, 0.01876, −0.01875, 0.01875, −0.01875, 0.01875, −0.01875, −0.01875, 0.01875, 0.01875, 0.01875, 0.01874, −0.01874, −0.01874, −0.01874, −0.01874, −0.01874, 0.01873, 0.01873, −0.01873, −0.01873, 0.01872, −0.01872, −0.01872, 0.01872, −0.01872, −0.01872, 0.01871, 0.01871, 0.01871, −0.01871, −0.01871, 0.01871, 0.01871, 0.0187, 0.0187, 0.0187, 0.0187, −0.0187, −0.0187, −0.0187, 0.0187, 0.01869, 0.01869, −0.01869, 0.01869, 0.01869, −0.01869, 0.01869, −0.01868, 0.01868, 0.01867, −0.01867, −0.01867, 0.01867, 0.01867, 0.01867, 0.01866, −0.01866, −0.01866, −0.01866, −0.01866, −0.01866, 0.01865, 0.01865, 0.01865, 0.01865, 0.01865, −0.01865, −0.01865, −0.01865, −0.01864, −0.01864, 0.01864, 0.01864, 0.01864, 0.01864, −0.01863, −0.01863, −0.01863, −0.01863, 0.01863, 0.01863, −0.01863, 0.01863, −0.01862, 0.01862, 0.01862, −0.01862, 0.01861, 0.01861, 0.01861, 0.01861, 0.01861, 0.01861, 0.0186, 0.0186, −0.0186, 0.0186, 0.0186, 0.0186, 0.0186, −0.0186, 0.01859, 0.01859, −0.01859, 0.01859, 0.01859, 0.01858, −0.01858, −0.01858, −0.01858, 0.01857, −0.01857, −0.01857, −0.01857, −0.01856, 0.01856, 0.01856, −0.01856, 0.01856, 0.01856, −0.01856, 0.01855, 0.01855, −0.01854, −0.01854, 0.01854, −0.01854, 0.01854, 0.01854, 0.01854, −0.01854, −0.01854, 0.01853, −0.01853, 0.01853, 0.01853, −0.01853, −0.01852, 0.01852, −0.01852, 0.01852, −0.01852, −0.01852, 0.01852, 0.01852, 0.01851, 0.01851, −0.01851, 0.01851, −0.01851, 0.01851, 0.0185, −0.0185, −0.0185, 0.0185, −0.0185, −0.0185, −0.01849, −0.01849, 0.01849, 0.01849, 0.01849, 0.01848, −0.01848, 0.01848, 0.01848, −0.01847, 0.01847, −0.01847, 0.01847, −0.01846, 0.01846, −0.01846, −0.01845, 0.01845, −0.01845, −0.01845, −0.01845, 0.01845, 0.01845, 0.01844, −0.01844, 0.01844, −0.01844, −0.01844, 0.01843, −0.01843, 0.01843, 0.01843, 0.01843, −0.01843, 0.01843, −0.01842, 0.01842, −0.01842, −0.01842, −0.01842, −0.01841, −0.01841, 0.01841, 0.01841, −0.01841, −0.0184, 0.0184, 0.0184, 0.0184, −0.01839, −0.01839, −0.01839, 0.01839, 0.01838, −0.01838, 0.01838, 0.01838, −0.01838, −0.01837, −0.01837, −0.01837, −0.01836, −0.01836, 0.01836, 0.01836, −0.01836, 0.01835, −0.01835, −0.01834, −0.01834, −0.01834, −0.01834, −0.01833, −0.01833, −0.01833, 0.01833, 0.01833, 0.01833, 0.01833, 0.01833, 0.01832, −0.01832, −0.01832, 0.01832, −0.01832, −0.01832, −0.01832, −0.01832, −0.01831, 0.01831, −0.01831, 0.01831, −0.01831, −0.01831, 0.0183, 0.0183, −0.0183, 0.01829, 0.01829, 0.01829, −0.01829, −0.01829, −0.01829, 0.01829, −0.01829, 0.01829, 0.01828, −0.01828, −0.01828, 0.01827, 0.01827, −0.01827, 0.01827, 0.01827, 0.01827, 0.01826, 0.01826, 0.01826, −0.01825, 0.01825, 0.01825, 0.01825, 0.01824, 0.01824, 0.01824, 0.01824, 0.01824, −0.01824, 0.01824, −0.01824, −0.01824, 0.01824, 0.01824, 0.01824, 0.01823, 0.01823, 0.01823, 0.01823, 0.01822, −0.01822, −0.01822, 0.01822, −0.01821, 0.01821, 0.01821, −0.01821, −0.0182, 0.0182, 0.0182, −0.01819, 0.01819, 0.01819, −0.01819, 0.01819, −0.01819, −0.01819, −0.01819, −0.01818, 0.01818, −0.01818, 0.01818, −0.01818, 0.01817, 0.01817, 0.01817, 0.01817, −0.01816, 0.01816, −0.01816, 0.01816, 0.01815, 0.01815, −0.01815, 0.01815, 0.01814, −0.01814, −0.01814, 0.01814, −0.01814, −0.01813, 0.01813, 0.01813, 0.01813, 0.01813, 0.01813, 0.01813, −0.01812, 0.01811, −0.01811, −0.01811, −0.01811, −0.0181, 0.0181, −0.0181, 0.0181, 0.0181, 0.01809, −0.01809, 0.01809, −0.01809, 0.01809, 0.01809, 0.01809, 0.01809, 0.01809, 0.01808, −0.01808, −0.01808, 0.01807, −0.01806, −0.01806, −0.01805, −0.01805, 0.01805, −0.01805, −0.01805, 0.01804, −0.01804, −0.01804, −0.01804, −0.01803, −0.01803, −0.01803, 0.01803, −0.01803, −0.01802, −0.01802, 0.01802, −0.01802, −0.01802, 0.01802, 0.01801, −0.01801, −0.01801, 0.01801, 0.018, −0.018, −0.018, 0.018, −0.018, 0.018, 0.018, −0.018, −0.018, 0.018, 0.01799, −0.01799, −0.01799, 0.01799, 0.01798, 0.01798, −0.01798, −0.01798, −0.01798, 0.01798, −0.01797, 0.01797, 0.01797, −0.01796, 0.01796, 0.01796, 0.01796, 0.01796, 0.01795, −0.01795, 0.01795, 0.01795, −0.01795, 0.01795, −0.01795, −0.01794, 0.01794, −0.01794, 0.01794, 0.01794, 0.01794, −0.01793, −0.01793, −0.01793, 0.01793, −0.01793, −0.01793, 0.01792, −0.01792, 0.01791, −0.01791, −0.01791, −0.01791, −0.0179, −0.0179, 0.0179, 0.0179, −0.01789, −0.01789, −0.01788, 0.01788, −0.01788, −0.01788, 0.01788, −0.01788, −0.01788, 0.01787, −0.01787, 0.01787, −0.01787, 0.01787, 0.01787, 0.01787, 0.01787, 0.01787, −0.01787, 0.01786, 0.01786, 0.01786, 0.01784, 0.01784, 0.01784, −0.01784, −0.01784, 0.01783, 0.01783, 0.01783, 0.01783, −0.01783, −0.01782, −0.01782, 0.01782, −0.01782, −0.01782, −0.01782, −0.01782, −0.01781, −0.01781, −0.01781, 0.01781, 0.01781, −0.0178, −0.0178, 0.0178, −0.01779, −0.01779, 0.01779, −0.01779, 0.01779, −0.01778, −0.01778, −0.01778, 0.01778, 0.01778, −0.01778, −0.01778, 0.01777, 0.01777, −0.01777, 0.01777, −0.01777, 0.01777, −0.01777, 0.01776, 0.01776, −0.01776, −0.01776, 0.01776, 0.01775, 0.01775, −0.01775, −0.01775, 0.01775, −0.01774, −0.01774, −0.01774, −0.01774, −0.01774, 0.01774, 0.01774, 0.01774, 0.01773, −0.01773, −0.01773, −0.01772, 0.01772, 0.01772, 0.01772, 0.01771, −0.0177, 0.0177, 0.0177, −0.0177, −0.0177, −0.0177, 0.0177, −0.01769, −0.01769, 0.01769, −0.01769, −0.01769, −0.01769, −0.01769, −0.01769, −0.01769, 0.01768, 0.01768, 0.01768, 0.01768, −0.01768, 0.01768, 0.01767, 0.01767, −0.01767, −0.01767, 0.01767, −0.01767, −0.01767, 0.01766, −0.01766, −0.01766, 0.01766, 0.01766, −0.01765, 0.01765, −0.01765, −0.01765, 0.01765, −0.01765, 0.01765, −0.01765, −0.01764, −0.01764, 0.01764, −0.01763, −0.01763, 0.01763, 0.01763, −0.01763, −0.01763, −0.01763, −0.01763, 0.01762, −0.01762, −0.01762, −0.01761, −0.01761, 0.01761, 0.01761, −0.0176, 0.0176, 0.0176, −0.0176, 0.0176, 0.0176, 0.01759, −0.01759, 0.01759, −0.01759, −0.01759, 0.01759, −0.01758, −0.01758, −0.01758, −0.01758, −0.01757, 0.01757, 0.01756, −0.01756, 0.01756, 0.01756, −0.01756, 0.01755, −0.01755, −0.01755, 0.01754, 0.01754, 0.01754, −0.01753, 0.01753, 0.01753, −0.01753, 0.01753, −0.01753, 0.01752, 0.01752, 0.01752, 0.01752, 0.01751, −0.01751, −0.0175, −0.0175, −0.0175, 0.0175, −0.0175, −0.0175, −0.0175, −0.0175, −0.0175, −0.01749, 0.01749, −0.01749, −0.01749, −0.01748, −0.01748, −0.01748, 0.01747, 0.01747, 0.01747, −0.01747, 0.01747, −0.01746, −0.01746, −0.01746, 0.01746, 0.01745, −0.01745, −0.01745, −0.01745, 0.01745, 0.01745, −0.01744, 0.01744, −0.01744, −0.01744, 0.01744, 0.01744, −0.01744, 0.01743, 0.01743, −0.01743, 0.01742, −0.01742, 0.01742, −0.01742, −0.01742, −0.01741, 0.01741, −0.01741, −0.01741, 0.01741, 0.01741, −0.0174, −0.0174, −0.0174, −0.0174, 0.0174, 0.01739, 0.01739, −0.01738, 0.01738, −0.01737, −0.01737, −0.01737, 0.01736, 0.01736, 0.01736, 0.01736, 0.01736, 0.01736, 0.01736, 0.01736, 0.01736, −0.01736, −0.01736, −0.01736, −0.01736, 0.01735, 0.01735, −0.01735, −0.01735, −0.01735, −0.01734, −0.01734, −0.01734, 0.01733, −0.01733, 0.01732, −0.01732, −0.01732, −0.01732, −0.01732, −0.01732, −0.01732, −0.01731, 0.01731, 0.01731, 0.01731, 0.01731, −0.01731, −0.0173, −0.0173, −0.0173, 0.0173, −0.0173, 0.01729, 0.01729, −0.01729, 0.01729, −0.01729, 0.01729, −0.01728, −0.01728, −0.01728, 0.01728, −0.01727, 0.01727, 0.01726, −0.01726, 0.01725, 0.01725, 0.01725, −0.01725, 0.01725, 0.01725, −0.01725, −0.01724, 0.01724, 0.01724, −0.01723, −0.01723, −0.01723, 0.01723, 0.01723, 0.01722, 0.01722, 0.01722, −0.01722, 0.01722, −0.01721, 0.01721, −0.01721, −0.01721, −0.0172, 0.0172, 0.0172, −0.0172, −0.0172, −0.0172, −0.01719, 0.01719, −0.01719, 0.01719, −0.01719, −0.01719, 0.01718, 0.01718, 0.01718, 0.01718, 0.01717, 0.01717, −0.01717, −0.01717, 0.01716, 0.01716, −0.01715, 0.01715, 0.01715, 0.01715, −0.01714, 0.01714, 0.01714, −0.01714, −0.01714, 0.01713, 0.01713, 0.01712, −0.01712, −0.01711, 0.01711, −0.01711, 0.0171, −0.0171, −0.0171, 0.0171, 0.0171, 0.0171, −0.0171, 0.0171, 0.0171, −0.0171, 0.01709, −0.01709, −0.01709, −0.01709, 0.01709, −0.01708, −0.01708, −0.01708, 0.01708, 0.01708, 0.01708, 0.01707, 0.01707, 0.01707, 0.01707, 0.01707, 0.01707, −0.01707, −0.01706, −0.01706, −0.01706, −0.01706, 0.01706, 0.01706, −0.01705, 0.01705, −0.01704, 0.01704, −0.01703, −0.01703, −0.01703, −0.01702, 0.01702, −0.01702, 0.01702, 0.01701, −0.01701, 0.01701, −0.017, 0.017, −0.01699, 0.01699, 0.01699, 0.01699, −0.01698, 0.01698, −0.01698, 0.01698, 0.01698, 0.01697, −0.01697, −0.01697, 0.01697, 0.01697, 0.01697, −0.01696, −0.01696, −0.01695, 0.01695, −0.01694, −0.01694, −0.01694, −0.01694, −0.01694, −0.01693, −0.01693, −0.01693, −0.01693, −0.01693, −0.01693, 0.01693, −0.01692, 0.01692, −0.01692, −0.01692, −0.01692, 0.01692, −0.01692, −0.01692, 0.01691, 0.01691, 0.01691, −0.0169, 0.0169, −0.0169, 0.0169, −0.0169, 0.0169, 0.01689, 0.01689, 0.01688, 0.01688, −0.01688, −0.01688, −0.01688, 0.01688, −0.01687, −0.01687, 0.01687, −0.01687, 0.01687, −0.01686, 0.01686, −0.01686, 0.01686, −0.01686, −0.01686, −0.01686, 0.01686, 0.01686, −0.01686, −0.01685, 0.01685, −0.01685, 0.01685, 0.01685, 0.01685, −0.01685, 0.01685, 0.01685, 0.01685, 0.01684, −0.01684, 0.01684, 0.01684, −0.01683, 0.01683, 0.01683, −0.01683, −0.01683, −0.01683, −0.01683, 0.01683, 0.01682, −0.01682, −0.01682, −0.01681, −0.01681, 0.01681, −0.01681, 0.01681, −0.01681, 0.01681, 0.01681, −0.01681, 0.0168, −0.0168, −0.0168, −0.0168, −0.01679, −0.01679, 0.01679, 0.01679, 0.01679, −0.01678, 0.01678, 0.01678, 0.01678, 0.01678, 0.01678, −0.01678, 0.01678, −0.01678, 0.01677, 0.01677, 0.01676, −0.01676, 0.01676, −0.01676, −0.01676, −0.01676, 0.01676, 0.01675, −0.01675, −0.01675, 0.01675, 0.01675, 0.01674, 0.01674, 0.01674, 0.01674, 0.01674, −0.01673, −0.01673, −0.01673, 0.01673, 0.01673, −0.01673, −0.01673, −0.01673, 0.01673, 0.01673, 0.01673, −0.01672, 0.01672, −0.01672, 0.01672, −0.01672, −0.01672, −0.01672, −0.01671, −0.01671, −0.01671, 0.01671, −0.0167, −0.0167, −0.0167, 0.0167, 0.0167, −0.0167, −0.01668, −0.01668, −0.01667, 0.01667, −0.01667, 0.01667, −0.01667, 0.01667, −0.01667, 0.01667, −0.01667, 0.01667, 0.01666, 0.01666, 0.01665, −0.01665, −0.01665, 0.01665, 0.01665, 0.01664, 0.01664, 0.01664, 0.01664, −0.01664, −0.01664, −0.01664, 0.01663, 0.01663, 0.01663, 0.01663, −0.01663, 0.01662, −0.01662, 0.01662, −0.01662, −0.01662, −0.01662, −0.01661, −0.01661, −0.01661, 0.01661, 0.01661, 0.01661, −0.01661, −0.0166, −0.0166, −0.0166, 0.01659, −0.01659, −0.01659, 0.01659, −0.01659, −0.01659, −0.01658, −0.01658, 0.01658, 0.01658, −0.01657, 0.01657, 0.01657, 0.01657, 0.01656, 0.01656, −0.01656, 0.01656, −0.01655, −0.01654, −0.01654, −0.01654, −0.01654, 0.01654, 0.01653, −0.01653, −0.01653, −0.01653, −0.01653, 0.01652, −0.01652, 0.01652, 0.01652, 0.01652, 0.01652, −0.01651, 0.01651, −0.01651, −0.01651, 0.01651, 0.01651, 0.01651, 0.01651, −0.0165, −0.0165, 0.0165, 0.0165, 0.0165, −0.01649, −0.01649, 0.01649, −0.01649, 0.01649, −0.01649, −0.01648, −0.01647, 0.01647, 0.01647, 0.01647, 0.01646, −0.01646, 0.01646, 0.01646, −0.01646, −0.01646, −0.01646, 0.01646, 0.01646, −0.01645, 0.01645, 0.01645, 0.01645, 0.01645, −0.01644, −0.01644, 0.01644, 0.01644, 0.01644, −0.01644, −0.01644, −0.01643, 0.01643, −0.01643, −0.01643, −0.01643, −0.01642, 0.01642, 0.01642, −0.01642, −0.01641, −0.01641, 0.01641, 0.01641, −0.01641, −0.01641, 0.01641, −0.01641, −0.01641, 0.01641, −0.0164, 0.0164, −0.0164, −0.0164, 0.0164, −0.0164, −0.01639, −0.01639, −0.01639, −0.01639, 0.01638, −0.01638, −0.01638, −0.01638, −0.01638, 0.01638, 0.01638, 0.01637, −0.01637, −0.01637, −0.01636, −0.01636, −0.01636, 0.01635, 0.01635, 0.01635, −0.01635, −0.01635, 0.01635, 0.01635, 0.01635, 0.01635, 0.01634, 0.01634, 0.01634, −0.01633, −0.01633, 0.01633, −0.01633, −0.01633, −0.01633, −0.01633, −0.01633, −0.01632, −0.01632, −0.01632, 0.01632, −0.01631, 0.01631, 0.01631, 0.0163, 0.0163, 0.0163, 0.0163, −0.01629, 0.01629, 0.01629, 0.01628, 0.01628, 0.01628, −0.01628, 0.01628, −0.01628, −0.01627, 0.01627, 0.01627, −0.01627, −0.01627, 0.01627, −0.01626, −0.01626, 0.01626, −0.01626, −0.01625, 0.01625, −0.01625, −0.01625, 0.01625, 0.01625, −0.01625, 0.01625, 0.01625, −0.01625, −0.01624, −0.01624, 0.01624, −0.01624, 0.01624, 0.01624, 0.01624, −0.01624, 0.01624, −0.01623, 0.01623, −0.01623, −0.01623, 0.01623, 0.01623, −0.01622, 0.01622, −0.01622, −0.01622, 0.01622, −0.01622, −0.01621, 0.01621, 0.01621, 0.01621, 0.0162, −0.0162, 0.0162, 0.0162, −0.0162, 0.0162, −0.01619, −0.01619, 0.01619, 0.01619, 0.01619, 0.01619, 0.01619, −0.01619, −0.01618, 0.01618, −0.01618, −0.01618, −0.01618, 0.01618, −0.01617, 0.01616, 0.01616, −0.01616, −0.01615, 0.01615, 0.01615, 0.01615, −0.01615, −0.01615, −0.01615, 0.01614, −0.01614, −0.01614, 0.01614, 0.01614, −0.01614, 0.01614, 0.01614, −0.01613, −0.01613, 0.01613, −0.01612, 0.01612, −0.01612, −0.01611, −0.01611, 0.01611, 0.01611, 0.01611, 0.01611, −0.01611, 0.0161, 0.0161, −0.0161, −0.0161, −0.0161, 0.0161, 0.0161, −0.0161, 0.0161, 0.01609, 0.01609, −0.01609, 0.01609, −0.01609, 0.01608, −0.01608, −0.01608, −0.01608, 0.01608, −0.01607, 0.01607, −0.01607, −0.01607, −0.01607, 0.01606, −0.01606, −0.01606, 0.01605, −0.01605, 0.01605, 0.01605, 0.01605, 0.01605, −0.01605, 0.01605, 0.01605, −0.01604, 0.01604, 0.01604, −0.01603, −0.01603, −0.01603, 0.01603, −0.01603, −0.01603, −0.01603, 0.01603, 0.01602, −0.01602, 0.01602, −0.01602, 0.01601, 0.01601, −0.01601, −0.01601, 0.01601, −0.01601, 0.01601, −0.016, −0.016, 0.016, −0.016, −0.016, 0.016, −0.01599, −0.01599, −0.01599, 0.01599, 0.01599, −0.01599, −0.01598, 0.01598, −0.01598, −0.01598, −0.01598, −0.01598, −0.01597, −0.01597, −0.01597, −0.01597, 0.01597, −0.01597, 0.01597, −0.01596, 0.01596, −0.01596, −0.01596, −0.01596, −0.01595, 0.01595, 0.01595, −0.01594, 0.01594, 0.01594, −0.01594, −0.01594, 0.01594, 0.01594, −0.01592, 0.01592, −0.01592, −0.01592, 0.01591, −0.01591, −0.01591, −0.01591, 0.0159, −0.0159, −0.01589, 0.01589, −0.01589, −0.01589, 0.01589, 0.01589, −0.01588, −0.01588, 0.01588, −0.01587, 0.01587, 0.01587, 0.01587, 0.01587, −0.01587, 0.01587, −0.01587, 0.01587, −0.01586, −0.01586, 0.01586, 0.01586, 0.01586, −0.01586, −0.01586, 0.01585, 0.01585, 0.01585, 0.01585, −0.01585, 0.01584, 0.01584, 0.01584, 0.01584, 0.01583, −0.01583, 0.01583, 0.01583, −0.01583, 0.01583, 0.01583, −0.01583, 0.01583, 0.01582, −0.01582, 0.01582, −0.01582, 0.01582, −0.01582, 0.01581, −0.01581, 0.01581, 0.01581, 0.01581, −0.0158, −0.0158, 0.0158, 0.0158, 0.01579, −0.01579, 0.01579, 0.01578, −0.01578, −0.01578, 0.01578, −0.01578, 0.01578, 0.01578, 0.01578, 0.01577, 0.01577, 0.01577, −0.01577, 0.01576, −0.01576, −0.01576, 0.01576, 0.01575, 0.01575, −0.01575, 0.01575, −0.01575, −0.01575, −0.01575, −0.01574, −0.01574, 0.01574, −0.01574, −0.01574, −0.01574, 0.01573, −0.01573, 0.01573, 0.01573, 0.01573, 0.01572, −0.01572, 0.01572, 0.01572, 0.01572, −0.01572, −0.01572, −0.01571, 0.01571, 0.01571, 0.01571, 0.01571, −0.0157, −0.0157, 0.0157, −0.0157, −0.0157, 0.01569, 0.01569, 0.01569, −0.01569, −0.01569, −0.01569, −0.01568, 0.01568, 0.01568, −0.01568, 0.01568, 0.01567, 0.01567, 0.01567, 0.01567, 0.01567, −0.01567, −0.01567, −0.01566, 0.01566, 0.01566, 0.01566, 0.01566, −0.01566, −0.01566, −0.01565, 0.01565, 0.01565, −0.01565, −0.01565, 0.01565, −0.01565, 0.01565, 0.01565, 0.01564, 0.01564, −0.01564, −0.01564, −0.01564, 0.01563, 0.01563, −0.01563, 0.01563, 0.01563, 0.01563, 0.01562, 0.01562, −0.01562, 0.01561, 0.01561, 0.01561, 0.01561, 0.01561, −0.01561, 0.01561, −0.01561, 0.01561, −0.0156, −0.0156, 0.0156, 0.0156, −0.0156, 0.0156, 0.01559, 0.01559, −0.01558, 0.01558, 0.01558, 0.01558, 0.01558, −0.01558, −0.01558, 0.01558, 0.01558, 0.01558, −0.01557, 0.01557, −0.01557, 0.01557, −0.01557, 0.01557, 0.01556, 0.01556, −0.01556, 0.01555, 0.01555, 0.01555, −0.01554, −0.01554, 0.01554, 0.01553, 0.01553, 0.01553, 0.01552, −0.01552, −0.01552, 0.01552, −0.01552, −0.0155, −0.0155, −0.0155, 0.0155, −0.01549, 0.01549, 0.01549, 0.01548, 0.01548, 0.01547, 0.01547, 0.01547, 0.01547, −0.01547, 0.01546, −0.01546, 0.01546, −0.01546, −0.01546, 0.01546, −0.01546, 0.01545, −0.01545, −0.01545, −0.01545, 0.01544, 0.01544, −0.01544, 0.01544, 0.01543, −0.01543, 0.01542, −0.01542, 0.01541, −0.01541, −0.0154, −0.0154, −0.0154, −0.0154, 0.0154, −0.01539, −0.01539, 0.01539, 0.01539, −0.01539, 0.01539, −0.01539, 0.01538, 0.01538, −0.01538, −0.01538, 0.01538, −0.01537, 0.01537, −0.01537, −0.01537, 0.01537, 0.01537, −0.01536, 0.01536, 0.01536, 0.01535, −0.01535, −0.01535, 0.01535, −0.01534, −0.01534, 0.01534, 0.01534, −0.01534, 0.01534, −0.01534, −0.01534, −0.01534, 0.01534, −0.01533, 0.01533, 0.01533, 0.01533, −0.01533, 0.01533, −0.01533, −0.01533, −0.01533, −0.01533, 0.01533, 0.01532, −0.01532, 0.01532, 0.01532, 0.01531, −0.01531, 0.01531, 0.01531, 0.01531, 0.0153, −0.0153, −0.0153, −0.0153, −0.0153, 0.0153, 0.0153, 0.0153, 0.0153, 0.0153, 0.0153, −0.01529, 0.01529, −0.01529, −0.01528, −0.01528, 0.01528, −0.01528, 0.01528, −0.01528, −0.01528, −0.01528, −0.01528, −0.01527, 0.01527, 0.01527, −0.01527, −0.01526, −0.01526, −0.01526, −0.01526, −0.01526, −0.01526, −0.01526, 0.01525, 0.01525, −0.01525, 0.01525, −0.01525, 0.01524, −0.01524, −0.01523, 0.01523, −0.01523, 0.01523, 0.01522, −0.01522, 0.01522, 0.01522, 0.01521, 0.01521, 0.01521, 0.01521, 0.01521, 0.01521, 0.01521, 0.01521, 0.01521, −0.01521, −0.01521, −0.01521, 0.0152, −0.0152, −0.0152, 0.0152, 0.0152, 0.01519, 0.01519, 0.01519, −0.01519, 0.01518, 0.01518, 0.01518, −0.01517, 0.01517, 0.01517, −0.01516, −0.01516, 0.01516, −0.01516, −0.01516, 0.01516, 0.01516, 0.01515, −0.01515, −0.01515, −0.01515, 0.01514, 0.01514, −0.01514, −0.01514, 0.01514, 0.01514, 0.01514, 0.01514, 0.01513, 0.01513, −0.01513, −0.01513, 0.01513, −0.01513, −0.01513, 0.01513, −0.01513, −0.01512, −0.01512, 0.01512, 0.01511, 0.01511, −0.01511, −0.01511, 0.01511, 0.0151, 0.0151, 0.0151, 0.0151, −0.0151, 0.0151, −0.0151, 0.0151, 0.01509, −0.01509, −0.01509, −0.01509, −0.01509, 0.01508, −0.01508, −0.01508, 0.01508, 0.01508, 0.01507, 0.01507, 0.01507, −0.01507, −0.01507, −0.01507, −0.01507, 0.01506, 0.01506, 0.01506, −0.01506, 0.01506, 0.01506, 0.01506, −0.01506, −0.01506, −0.01506, 0.01506, −0.01505, −0.01505, 0.01505, −0.01505, −0.01505, −0.01504, −0.01504, −0.01503, −0.01503, 0.01503, −0.01503, 0.01503, 0.01503, 0.01503, 0.01502, 0.01502, 0.01502, 0.01502, −0.01502, 0.01501, 0.01501, 0.01501, −0.01501, 0.01501, −0.01501, 0.01501, −0.015, 0.015, −0.015, 0.015, −0.015, −0.015, −0.015, 0.015, 0.01499, 0.01499, −0.01499, 0.01499, −0.01499, −0.01499, 0.01498, −0.01498, 0.01497, 0.01497, 0.01497, −0.01497, −0.01497, −0.01496, 0.01496, 0.01496, −0.01496, 0.01496, 0.01496, 0.01496, −0.01495, 0.01495, 0.01495, −0.01495, −0.01495, 0.01495, 0.01495, −0.01494, 0.01494, 0.01494, −0.01493, −0.01493, −0.01493, −0.01493, 0.01493, −0.01492, −0.01492, −0.01492, −0.01492, −0.01492, −0.01491, 0.01491, 0.01491, −0.01491, 0.01491, −0.01491, 0.01491, −0.0149, −0.0149, 0.0149, 0.0149, −0.01489, 0.01489, 0.01489, −0.01488, 0.01488, −0.01488, 0.01488, −0.01488, −0.01488, 0.01488, 0.01488, 0.01487, 0.01486, −0.01486, 0.01486, 0.01486, −0.01486, 0.01486, 0.01486, 0.01486, −0.01485, 0.01485, 0.01485, −0.01485, −0.01485, 0.01485, −0.01485, 0.01485, −0.01484, −0.01484, −0.01484, 0.01483, −0.01483, 0.01483, 0.01483, 0.01483, −0.01483, 0.01482, −0.01482, −0.01482, 0.01482, −0.01482, 0.01482, −0.01481, −0.01481, −0.01481, −0.01481, 0.01481, −0.01481, −0.01481, −0.01481, 0.01481, 0.01481, −0.0148, −0.0148, 0.0148, 0.0148, 0.0148, 0.0148, −0.0148, −0.01479, 0.01479, 0.01479, 0.01479, −0.01479, −0.01479, 0.01479, −0.01479, 0.01478, −0.01478, −0.01478, 0.01478, 0.01478, 0.01477, 0.01477, −0.01477, −0.01477, −0.01476, −0.01476, −0.01476, −0.01476, −0.01476, 0.01476, 0.01476, 0.01476, −0.01476, −0.01475, 0.01475, 0.01475, −0.01475, −0.01475, −0.01475, −0.01475, −0.01474, −0.01474, −0.01474, 0.01474, −0.01474, −0.01473, 0.01473, 0.01473, 0.01473, 0.01473, 0.01473, 0.01472, 0.01472, 0.01472, 0.01472, −0.01472, −0.01471, 0.01471, −0.01471, −0.01471, −0.01471, −0.01471, −0.01471, −0.0147, 0.0147, 0.0147, −0.0147, 0.0147, 0.0147, 0.01469, −0.01469, −0.01469, −0.01469, −0.01469, −0.01469, −0.01469, 0.01469, −0.01468, 0.01468, 0.01468, 0.01468, −0.01468, −0.01468, −0.01468, −0.01468, −0.01467, 0.01467, 0.01467, −0.01467, −0.01467, −0.01466, −0.01466, 0.01466, 0.01466, 0.01466, 0.01465, 0.01465, 0.01465, 0.01465, −0.01465, 0.01465, −0.01465, −0.01465, 0.01465, 0.01464, 0.01464, 0.01464, −0.01464, −0.01464, −0.01464, 0.01463, −0.01463, 0.01463, −0.01462, 0.01462, −0.01462, −0.01462, 0.01462, 0.01462, −0.01461, 0.01461, −0.01461, 0.01461, 0.01461, −0.01461, −0.0146, −0.0146, 0.0146, −0.0146, 0.0146, 0.0146, −0.01459, −0.01459, 0.01459, 0.01458, 0.01458, 0.01458, −0.01458, 0.01458, −0.01458, 0.01458, 0.01458, 0.01457, 0.01457, 0.01457, 0.01457, −0.01457, 0.01457, 0.01457, −0.01457, −0.01457, 0.01456, 0.01456, −0.01456, −0.01456, −0.01456, 0.01456, 0.01456, −0.01455, −0.01455, −0.01455, 0.01454, −0.01454, 0.01454, 0.01454, 0.01454, 0.01454, 0.01454, 0.01454, 0.01454, 0.01454, −0.01453, 0.01453, 0.01452, −0.01452, 0.01452, −0.01452, 0.01452, −0.01451, 0.01451, 0.01451, −0.01451, 0.01451, 0.0145, −0.0145, 0.0145, −0.0145, 0.0145, −0.01449, 0.01449, −0.01449, 0.01449, 0.01448, −0.01448, −0.01448, 0.01448, −0.01447, −0.01447, −0.01447, −0.01447, 0.01447, 0.01447, 0.01447, 0.01447, −0.01447, −0.01446, −0.01446, 0.01446, −0.01446, −0.01446, 0.01445, −0.01445, −0.01445, 0.01445, −0.01445, 0.01444, −0.01443, −0.01443, 0.01443, −0.01443, 0.01443, −0.01443, 0.01443, −0.01443, 0.01443, −0.01443, 0.01443, 0.01443, −0.01443, 0.01442, −0.01442, −0.01442, 0.01442, 0.01442, 0.01442, −0.01442, 0.01441, −0.01441, −0.01441, −0.01441, −0.01441, 0.01441, −0.0144, −0.0144, 0.0144, −0.0144, 0.0144, 0.0144, −0.01439, −0.01439, 0.01439, 0.01439, −0.01439, 0.01439, −0.01439, 0.01438, −0.01438, −0.01438, 0.01438, 0.01437, 0.01437, −0.01437, 0.01437, −0.01437, 0.01436, 0.01436, −0.01436, 0.01436, −0.01436, −0.01436, 0.01436, −0.01436, 0.01436, 0.01436, −0.01436, −0.01435, 0.01435, 0.01435, 0.01435, 0.01435, −0.01435, 0.01434, 0.01434, −0.01434, −0.01434, −0.01434, 0.01434, −0.01433, −0.01433, −0.01432, −0.01432, −0.01432, 0.01432, 0.01432, −0.01432, −0.01432, −0.01431, 0.01431, −0.01431, 0.01431, −0.01431, 0.01431, −0.0143, −0.0143, −0.0143, 0.0143, −0.0143, 0.0143, 0.0143, 0.01429, −0.01429, −0.01429, 0.01429, 0.01429, −0.01429, −0.01428, −0.01428, −0.01428, 0.01428, −0.01428, 0.01427, −0.01427, 0.01427, −0.01427, 0.01427, −0.01427, −0.01426, 0.01426, 0.01426, 0.01425, 0.01425, 0.01425, 0.01425, 0.01425, 0.01424, 0.01424, 0.01424, 0.01423, −0.01423, 0.01423, −0.01423, 0.01423, 0.01423, 0.01423, 0.01423, 0.01422, 0.01422, −0.01422, 0.01422, 0.01422, 0.01422, 0.01422, 0.01421, −0.01421, −0.01421, −0.01421, 0.01421, 0.01421, −0.0142, 0.0142, 0.0142, −0.01419, −0.01419, 0.01419, −0.01419, −0.01419, −0.01419, 0.01418, −0.01418, 0.01418, 0.01418, 0.01418, 0.01418, 0.01418, −0.01418, 0.01418, −0.01418, 0.01417, 0.01417, −0.01417, −0.01417, −0.01417, 0.01417, −0.01417, −0.01417, 0.01416, −0.01416, 0.01416, 0.01415, −0.01415, 0.01415, 0.01415, −0.01415, 0.01415, −0.01415, 0.01415, 0.01415, −0.01414, −0.01414, 0.01414, 0.01414, 0.01414, 0.01414, −0.01414, 0.01414, −0.01414, 0.01414, −0.01414, 0.01413, 0.01413, −0.01413, −0.01413, 0.01413, 0.01413, 0.01413, 0.01413, 0.01413, −0.01412, 0.01412, 0.01412, −0.01412, −0.01412, 0.01411, 0.01411, −0.01411, 0.01411, −0.01411, −0.01411, 0.01411, −0.01411, −0.01411, 0.0141, 0.0141, −0.0141, −0.0141, −0.0141, 0.0141, −0.01409, −0.01409, 0.01409, 0.01409, −0.01409, −0.01409, 0.01409, 0.01409, −0.01409, 0.01409, −0.01409, −0.01409, −0.01408, 0.01408, −0.01408, 0.01408, 0.01408, 0.01408, −0.01407, 0.01407, 0.01407, −0.01407, 0.01407, 0.01407, −0.01407, 0.01407, −0.01407, −0.01407, −0.01406, −0.01406, 0.01406, 0.01406, −0.01406, −0.01406, 0.01406, −0.01405, 0.01405, 0.01405, 0.01405, −0.01404, 0.01404, 0.01404, 0.01404, −0.01404, 0.01404, −0.01404, −0.01403, −0.01403, 0.01403, −0.01403, −0.01403, −0.01403, −0.01403, −0.01403, −0.01402, 0.01402, −0.01402, −0.01402, 0.01402, 0.01402, −0.01401, −0.01401, −0.01401, −0.01401, 0.01401, −0.01401, −0.01401, 0.014, 0.014, 0.014, 0.014, −0.014, −0.01399, −0.01399, −0.01399, −0.01399, 0.01399, −0.01399, −0.01399, 0.01398, −0.01398, 0.01398, −0.01398, 0.01398, −0.01397, −0.01396, −0.01396, 0.01396, 0.01396, 0.01396, −0.01396, 0.01395, −0.01395, 0.01395, 0.01395, 0.01395, −0.01395, −0.01395, 0.01394, −0.01394, 0.01394, −0.01394, −0.01394, 0.01394, 0.01394, 0.01394, −0.01393, −0.01393, 0.01393, 0.01393, 0.01393, 0.01393, 0.01393, −0.01393, 0.01392, 0.01392, 0.01392, −0.01392, −0.01392, −0.01392, −0.01392, −0.01392, −0.01391, 0.01391, −0.01391, 0.01391, −0.01391, 0.01391, −0.01391, −0.0139, −0.0139, −0.0139, −0.0139, 0.0139, 0.0139, 0.0139, −0.0139, −0.01389, 0.01389, −0.01389, −0.01389, 0.01389, 0.01388, 0.01388, −0.01388, 0.01388, 0.01388, −0.01388, −0.01387, 0.01387, 0.01387, 0.01387, −0.01387, 0.01386, 0.01386, 0.01386, −0.01385, 0.01385, −0.01385, −0.01385, 0.01385, −0.01385, 0.01385, −0.01385, 0.01385, 0.01385, −0.01385, 0.01385, 0.01384, 0.01384, −0.01384, −0.01384, 0.01384, −0.01384, −0.01383, −0.01383, −0.01383, 0.01383, 0.01383, 0.01382, 0.01382, −0.01381, −0.01381, −0.01381, 0.01381, 0.01381, 0.01381, 0.01381, 0.01381, −0.0138, 0.0138, 0.0138, 0.0138, −0.0138, −0.0138, −0.0138, 0.0138, −0.0138, −0.01379, −0.01379, −0.01379, 0.01379, −0.01378, 0.01378, 0.01378, −0.01378, 0.01378, −0.01377, −0.01377, −0.01377, −0.01377, 0.01376, −0.01376, 0.01376, −0.01376, 0.01376, 0.01376, −0.01375, −0.01375, 0.01375, −0.01375, 0.01375, 0.01375, −0.01375, 0.01375, −0.01374, 0.01374, −0.01374, 0.01374, −0.01374, −0.01374, −0.01373, −0.01373, −0.01373, 0.01373, −0.01373, 0.01372, 0.01372, 0.01372, 0.01372, −0.01371, 0.01371, −0.01371, −0.01371, 0.01371, −0.01371, 0.01371, 0.0137, −0.0137, −0.0137, −0.0137, 0.01369, −0.01369, 0.01369, 0.01368, −0.01368, −0.01368, −0.01368, 0.01368, −0.01368, −0.01367, 0.01367, 0.01367, −0.01367, 0.01367, −0.01366, −0.01366, −0.01366, 0.01366, −0.01366, 0.01366, −0.01366, −0.01366, 0.01366, 0.01366, −0.01365, −0.01365, −0.01365, −0.01365, −0.01365, 0.01364, −0.01364, 0.01364, −0.01363, −0.01363, 0.01363, 0.01363, 0.01363, −0.01363, 0.01362, 0.01362, 0.01362, −0.01362, 0.01361, 0.01361, 0.01361, 0.01361, −0.01361, 0.01361, −0.0136, 0.0136, 0.0136, −0.0136, −0.0136, 0.0136, 0.0136, 0.0136, −0.0136, −0.01359, −0.01359, −0.01359, 0.01359, 0.01359, 0.01358, 0.01358, 0.01358, 0.01358, 0.01358, −0.01357, 0.01357, 0.01357, 0.01357, 0.01357, 0.01357, −0.01357, −0.01357, −0.01357, 0.01357, 0.01356, 0.01356, 0.01356, −0.01356, −0.01356, −0.01355, 0.01355, 0.01355, 0.01355, −0.01355, −0.01355, −0.01355, −0.01354, −0.01354, −0.01354, −0.01354, 0.01354, −0.01354, −0.01354, −0.01354, −0.01354, −0.01354, 0.01353, 0.01353, 0.01353, −0.01353, 0.01352, 0.01352, 0.01352, −0.01352, 0.01352, −0.01352, −0.01352, −0.01352, −0.01352, −0.01351, 0.01351, 0.01351, 0.01351, 0.01351, 0.01351, −0.0135, 0.0135, −0.0135, −0.0135, −0.01349, −0.01349, 0.01349, −0.01348, 0.01348, 0.01348, −0.01348, 0.01348, −0.01347, 0.01347, 0.01347, 0.01347, −0.01347, 0.01347, 0.01347, 0.01346, −0.01346, −0.01346, −0.01346, 0.01346, 0.01346, 0.01346, 0.01346, 0.01346, −0.01345, 0.01345, 0.01345, 0.01344, 0.01344, 0.01344, −0.01344, 0.01344, −0.01343, −0.01343, 0.01343, 0.01343, −0.01343, 0.01342, −0.01342, −0.01342, −0.01342, −0.01342, −0.01342, 0.01342, −0.01342, −0.01342, −0.01342, −0.01341, −0.01341, −0.01341, −0.01341, 0.01341, 0.01341, 0.01341, 0.01341, 0.0134, −0.0134, −0.0134, 0.0134, 0.0134, −0.0134, 0.0134, −0.0134, 0.0134, −0.01339, 0.01339, 0.01339, −0.01339, 0.01339, 0.01338, −0.01338, 0.01338, −0.01338, −0.01338, −0.01337, −0.01337, 0.01337, −0.01337, −0.01337, 0.01337, 0.01337, −0.01337, −0.01337, 0.01337, −0.01336, 0.01336, −0.01336, −0.01336, 0.01336, −0.01336, −0.01336, −0.01335, 0.01335, 0.01335, −0.01335, 0.01335, −0.01334, −0.01334, 0.01334, 0.01333, −0.01333, −0.01333, 0.01332, −0.01332, 0.01332, −0.01332, 0.01332, −0.01332, −0.01331, 0.01331, 0.01331, −0.01331, 0.01331, 0.01331, 0.01331, 0.01331, 0.0133, 0.0133, 0.0133, 0.0133, 0.0133, −0.0133, −0.01329, −0.01329, −0.01329, −0.01329, −0.01329, −0.01329, −0.01329, 0.01329, −0.01329, −0.01329, −0.01329, −0.01329, −0.01328, 0.01328, −0.01328, −0.01328, 0.01328, −0.01327, 0.01327, 0.01327, −0.01327, −0.01326, −0.01326, 0.01326, −0.01326, −0.01326, 0.01326, −0.01326, 0.01326, 0.01326, −0.01325, 0.01325, −0.01325, −0.01325, −0.01325, 0.01325, 0.01324, −0.01324, −0.01324, 0.01324, 0.01324, 0.01324, 0.01322, −0.01322, 0.01321, −0.01321, 0.0132, 0.0132, −0.0132, −0.0132, 0.0132, −0.01319, −0.01319, 0.01319, −0.01319, −0.01319, −0.01319, 0.01318, 0.01318, −0.01318, 0.01318, −0.01318, 0.01318, 0.01318, −0.01318, 0.01318, −0.01318, 0.01317, 0.01317, −0.01317, −0.01317, 0.01317, 0.01317, 0.01317, −0.01317, −0.01317, 0.01317, 0.01317, 0.01316, 0.01316, −0.01316, −0.01316, 0.01316, −0.01316, −0.01316, 0.01316, −0.01315, −0.01315, −0.01314, 0.01314, 0.01314, −0.01314, −0.01314, −0.01313, −0.01313, 0.01313, 0.01313, −0.01312, −0.01312, 0.01312, 0.01312, 0.01311, −0.01311, −0.01311, 0.01311, 0.01311, −0.0131, −0.0131, −0.0131, −0.0131, −0.0131, 0.0131, 0.0131, −0.0131, −0.0131, 0.01309, −0.01309, −0.01309, 0.01309, 0.01308, −0.01308, −0.01308, 0.01308, −0.01308, −0.01308, 0.01308, 0.01308, −0.01308, −0.01308, 0.01308, 0.01307, 0.01307, −0.01307, −0.01307, −0.01307, −0.01307, 0.01307, 0.01306, −0.01306, 0.01306, −0.01306, −0.01305, 0.01305, −0.01304, 0.01304, −0.01304, 0.01304, 0.01304, 0.01304, −0.01304, 0.01304, 0.01303, −0.01303, −0.01303, −0.01303, −0.01302, −0.01302, 0.01301, 0.01301, 0.01301, −0.01301, 0.01301, −0.01301, 0.01301, 0.01301, 0.01301, −0.01301, 0.01301, −0.013, −0.013, 0.013, −0.01299, −0.01299, 0.01299, 0.01299, 0.01299, −0.01299, 0.01299, −0.01299, 0.01298, 0.01298, 0.01298, 0.01298, −0.01298, 0.01297, −0.01297, 0.01297, 0.01296, 0.01296, 0.01296, 0.01296, 0.01296, 0.01296, −0.01296, 0.01295, −0.01295, 0.01295, −0.01295, −0.01295, 0.01295, −0.01294, −0.01294, −0.01294, 0.01294, 0.01294, 0.01294, −0.01293, −0.01293, 0.01293, 0.01293, 0.01293, 0.01293, −0.01293, 0.01293, 0.01293, 0.01293, −0.01293, −0.01292, 0.01292, −0.01292, −0.01292, 0.01292, 0.01292, −0.01292, 0.01292, −0.01291, 0.01291, −0.01291, −0.01291, −0.01291, 0.01291, 0.0129, 0.0129, −0.0129, −0.0129, −0.01289, −0.01289, 0.01289, 0.01289, −0.01289, −0.01289, −0.01289, −0.01288, 0.01288, 0.01288, 0.01288, 0.01288, −0.01288, −0.01288, −0.01288, 0.01288, 0.01288, 0.01287, −0.01287, −0.01287, −0.01287, −0.01287, 0.01287, −0.01287, −0.01286, 0.01286, 0.01286, 0.01285, 0.01285, 0.01285, 0.01285, 0.01285, 0.01284, 0.01284, −0.01284, 0.01284, 0.01284, −0.01284, 0.01284, −0.01283, −0.01283, 0.01283, 0.01283, −0.01283, 0.01283, −0.01283, −0.01283, 0.01283, 0.01283, −0.01283, 0.01282, −0.01282, −0.01282, 0.01282, −0.01282, 0.01282, 0.01281, 0.01281, 0.01281, 0.01281, −0.01281, 0.01281, 0.01281, 0.0128, 0.0128, −0.0128, 0.0128, −0.0128, −0.01279, 0.01279, 0.01279, 0.01279, −0.01279, −0.01279, 0.01279, 0.01278, 0.01278, −0.01277, −0.01277, −0.01277, −0.01277, −0.01277, 0.01277, 0.01276, 0.01276, −0.01276, −0.01276, 0.01276, −0.01276, −0.01276, −0.01276, −0.01276, 0.01276, −0.01276, 0.01276, 0.01276, 0.01276, 0.01275, 0.01275, 0.01275, 0.01275, −0.01275, 0.01275, 0.01275, −0.01275, −0.01275, −0.01274, 0.01273, 0.01273, 0.01273, 0.01272, −0.01272, −0.01272, −0.01272, 0.01272, −0.01272, −0.01272, 0.01272, 0.01271, −0.01271, 0.01271, 0.01271, −0.01271, 0.01271, 0.01271, −0.01271, −0.0127, −0.0127, 0.0127, −0.0127, −0.0127, −0.01269, −0.01269, −0.01269, −0.01269, 0.01269, 0.01269, 0.01269, −0.01269, 0.01269, 0.01269, −0.01269, −0.01268, −0.01268, −0.01268, 0.01268, 0.01268, 0.01268, 0.01267, 0.01267, −0.01267, 0.01267, 0.01267, −0.01267, 0.01266, −0.01266, 0.01266, 0.01266, 0.01266, 0.01266, 0.01265, −0.01265, 0.01265, 0.01265, −0.01264, 0.01264, 0.01264, 0.01264, 0.01264, 0.01263, −0.01263, 0.01263, 0.01263, 0.01263, −0.01263, −0.01263, −0.01263, −0.01262, 0.01262, 0.01262, 0.01262, −0.01262, −0.01261, 0.01261, 0.01261, 0.01261, 0.01261, 0.01261, −0.01261, −0.01261, 0.0126, −0.0126, −0.0126, 0.0126, −0.0126, −0.0126, −0.0126, 0.0126, −0.0126, 0.0126, −0.01259, −0.01259, 0.01259, 0.01259, 0.01259, −0.01258, 0.01258, −0.01258, 0.01258, 0.01258, −0.01258, −0.01258, 0.01258, −0.01258, 0.01258, −0.01258, 0.01257, −0.01257, −0.01257, −0.01257, 0.01256, −0.01256, −0.01256, 0.01256, 0.01256, −0.01256, −0.01256, −0.01256, 0.01255, 0.01255, 0.01255, 0.01255, 0.01254, −0.01254, 0.01254, −0.01254, −0.01254, 0.01254, 0.01254, 0.01254, −0.01253, 0.01253, −0.01253, −0.01253, −0.01253, −0.01252, 0.01252, −0.01252, 0.01252, −0.01252, −0.01252, −0.01252, −0.01252, −0.01252, 0.01252, 0.01251, −0.01251, −0.01251, 0.01251, 0.01251, 0.01251, 0.0125, 0.0125, −0.0125, −0.0125, 0.0125, 0.0125, 0.0125, −0.01249, −0.01249, 0.01249, −0.01249, 0.01249, 0.01249, 0.01248, 0.01248, −0.01248, 0.01248, −0.01248, 0.01248, 0.01248, −0.01248, −0.01247, 0.01246, 0.01246, −0.01246, 0.01246, 0.01246, 0.01246, 0.01246, 0.01245, −0.01245, 0.01245, 0.01245, −0.01245, 0.01245, 0.01245, 0.01245, −0.01245, 0.01245, 0.01244, −0.01244, 0.01244, −0.01244, −0.01244, 0.01244, 0.01243, −0.01243, −0.01243, −0.01243, −0.01243, 0.01243, −0.01243, −0.01243, 0.01242, −0.01242, 0.01242, 0.01242, 0.01241, 0.01241, −0.01241, 0.01241, 0.01241, 0.01241, −0.01241, −0.0124, 0.0124, −0.0124, −0.0124, −0.0124, −0.0124, −0.0124, −0.0124, −0.0124, −0.0124, −0.0124, −0.0124, 0.01239, 0.01239, 0.01239, −0.01239, 0.01239, −0.01239, −0.01239, 0.01238, 0.01238, 0.01238, 0.01238, 0.01238, −0.01237, 0.01237, −0.01237, 0.01237, 0.01237, 0.01237, 0.01237, −0.01237, −0.01236, 0.01236, −0.01236, 0.01236, 0.01236, −0.01236, −0.01236, 0.01236, −0.01236, 0.01235, 0.01235, 0.01235, 0.01235, 0.01235, −0.01235, 0.01235, 0.01235, 0.01235, 0.01234, 0.01234, 0.01234, −0.01234, −0.01234, −0.01234, 0.01234, 0.01234, −0.01234, −0.01233, −0.01233, 0.01233, 0.01233, −0.01233, 0.01233, 0.01233, 0.01233, −0.01232, −0.01232, 0.01232, −0.01232, −0.01232, −0.01232, −0.01232, 0.01232, −0.01231, 0.01231, −0.01231, 0.01231, −0.01231, 0.01231, 0.01231, −0.01231, 0.01231, 0.01231, −0.01231, 0.0123, 0.0123, −0.01229, −0.01229, 0.01229, 0.01229, −0.01228, −0.01228, −0.01228, −0.01228, 0.01228, −0.01228, −0.01227, 0.01227, −0.01227, −0.01227, 0.01227, 0.01227, −0.01227, −0.01227, −0.01227, −0.01226, −0.01226, −0.01226, 0.01226, −0.01226, 0.01226, −0.01226, 0.01226, 0.01226, 0.01226, 0.01225, −0.01225, 0.01225, 0.01225, 0.01225, −0.01224, 0.01224, −0.01224, 0.01224, 0.01224, −0.01224, 0.01224, −0.01223, −0.01223, 0.01223, −0.01223, 0.01223, −0.01223, 0.01222, 0.01222, −0.01222, 0.01222, −0.01222, −0.01221, 0.01221, −0.01221, 0.01221, 0.01221, −0.01221, 0.01221, 0.01221, 0.0122, 0.0122, −0.0122, 0.0122, −0.0122, −0.0122, 0.0122, 0.0122, −0.01219, 0.01219, −0.01219, 0.01218, −0.01218, 0.01218, −0.01218, −0.01218, −0.01218, 0.01218, 0.01217, 0.01217, −0.01217, −0.01217, −0.01217, −0.01217, 0.01217, −0.01217, −0.01216, 0.01216, −0.01216, −0.01216, 0.01216, 0.01216, 0.01215, −0.01215, −0.01215, −0.01215, −0.01215, 0.01215, −0.01215, 0.01214, 0.01214, −0.01214, −0.01214, −0.01214, 0.01213, 0.01213, −0.01213, −0.01213, 0.01213, −0.01213, −0.01213, 0.01212, 0.01212, 0.01212, −0.01212, 0.01212, 0.01212, 0.01212, 0.01212, 0.01212, 0.01212, 0.01211, 0.01211, −0.01211, −0.01211, −0.01211, 0.01211, 0.01211, 0.01211, 0.01211, 0.01211, 0.0121, 0.0121, −0.0121, −0.0121, −0.0121, −0.0121, −0.0121, 0.0121, 0.0121, 0.0121, −0.0121, 0.01209, −0.01209, 0.01209, −0.01209, 0.01209, −0.01209, −0.01208, −0.01208, 0.01208, −0.01208, −0.01208, 0.01208, 0.01207, −0.01207, −0.01207, 0.01207, 0.01207, −0.01207, 0.01207, −0.01206, 0.01206, 0.01206, 0.01206, 0.01206, −0.01206, 0.01206, −0.01206, −0.01205, −0.01205, −0.01205, −0.01205, 0.01205, 0.01205, 0.01205, 0.01205, −0.01205, 0.01205, 0.01205, 0.01205, 0.01204, −0.01204, 0.01204, −0.01204, 0.01204, 0.01203, −0.01203, 0.01203, 0.01203, −0.01203, 0.01203, −0.01203, −0.01202, −0.01202, −0.01202, −0.01202, −0.01202, −0.01202, −0.01202, 0.01202, −0.01202, −0.01202, −0.01201, −0.01201, −0.01201, 0.01201, −0.01201, 0.01201, −0.01201, −0.01201, −0.012, −0.012, 0.012, −0.012, 0.012, 0.012, −0.012, −0.012, 0.012, −0.01199, −0.01199, 0.01199, 0.01199, 0.01199, 0.01199, 0.01198, −0.01198, 0.01198, 0.01198, 0.01198, 0.01198, 0.01198, 0.01198, 0.01198, −0.01197, 0.01197, −0.01197, 0.01197, −0.01197, −0.01197, −0.01197, −0.01197, 0.01197, 0.01197, 0.01196, 0.01196, −0.01196, −0.01196, 0.01196, 0.01196, 0.01196, 0.01196, 0.01196, 0.01196, 0.01196, −0.01196, −0.01196, 0.01195, 0.01195, −0.01195, 0.01195, 0.01194, 0.01194, −0.01194, 0.01194, −0.01193, 0.01193, −0.01193, 0.01193, −0.01192, 0.01192, −0.01192, 0.01192, 0.01192, 0.01191, 0.01191, −0.01191, −0.01191, 0.01191, 0.01191, −0.01191, −0.0119, 0.0119, −0.0119, −0.0119, −0.0119, 0.0119, −0.0119, −0.0119, 0.0119, −0.0119, 0.01189, −0.01189, 0.01189, 0.01189, 0.01189, 0.01189, −0.01188, −0.01188, −0.01188, −0.01188, −0.01188, 0.01188, 0.01188, −0.01188, −0.01188, 0.01188, −0.01188, 0.01187, −0.01187, −0.01187, 0.01187, −0.01187, 0.01187, 0.01187, −0.01187, −0.01187, −0.01187, −0.01186, −0.01186, −0.01186, 0.01186, 0.01186, −0.01186, 0.01186, −0.01186, −0.01185, 0.01185, 0.01185, −0.01185, 0.01185, 0.01185, 0.01185, 0.01185, −0.01184, 0.01184, −0.01184, 0.01184, −0.01184, 0.01184, −0.01184, −0.01184, 0.01184, −0.01183, 0.01183, 0.01183, 0.01183, 0.01183, −0.01182, 0.01182, 0.01182, −0.01182, −0.01182, 0.01182, −0.01181, 0.01181, 0.01181, −0.01181, 0.01181, 0.01181, 0.01181, −0.0118, −0.0118, 0.0118, 0.0118, 0.0118, 0.01179, 0.01179, −0.01178, 0.01178, −0.01178, 0.01178, 0.01178, −0.01177, −0.01177, 0.01177, 0.01177, 0.01176, −0.01176, 0.01176, −0.01176, −0.01176, −0.01175, 0.01175, 0.01175, 0.01175, −0.01175, 0.01175, 0.01175, −0.01175, −0.01174, 0.01174, 0.01174, 0.01174, 0.01174, −0.01174, 0.01174, 0.01174, −0.01174, 0.01174, −0.01174, 0.01174, 0.01173, 0.01173, 0.01173, 0.01173, 0.01172, 0.01172, −0.01172, 0.01172, −0.01172, −0.01172, −0.01172, 0.01172, 0.01171, 0.01171, 0.01171, −0.01171, −0.01171, 0.01171, −0.01171, −0.0117, −0.0117, 0.0117, −0.0117, 0.0117, −0.0117, 0.0117, −0.01169, 0.01169, −0.01169, 0.01169, 0.01169, −0.01169, 0.01169, 0.01169, −0.01168, 0.01168, 0.01168, 0.01168, −0.01168, −0.01168, 0.01167, −0.01167, −0.01167, −0.01167, −0.01167, 0.01166, 0.01166, 0.01166, −0.01166, 0.01166, 0.01166, −0.01166, 0.01166, 0.01166, 0.01166, −0.01165, −0.01165, −0.01165, 0.01165, 0.01165, −0.01165, −0.01164, 0.01164, 0.01164, 0.01164, −0.01164, −0.01164, −0.01164, 0.01164, 0.01164, −0.01163, −0.01163, −0.01163, 0.01163, −0.01163, 0.01162, 0.01162, −0.01162, −0.01162, 0.01162, 0.01162, 0.01161, 0.01161, −0.01161, −0.01161, −0.01161, 0.01161, −0.01161, −0.01161, −0.0116, 0.0116, 0.0116, −0.0116, 0.0116, −0.0116, 0.0116, −0.0116, −0.0116, 0.0116, 0.0116, −0.0116, 0.01159, 0.01159, −0.01159, 0.01159, −0.01159, −0.01159, −0.01159, 0.01159, −0.01159, −0.01159, −0.01159, −0.01159, 0.01159, 0.01158, 0.01158, 0.01158, −0.01158, 0.01158, 0.01158, 0.01158, −0.01158, −0.01157, −0.01157, −0.01157, 0.01157, −0.01157, −0.01157, 0.01157, −0.01156, −0.01156, −0.01156, −0.01156, −0.01156, 0.01156, 0.01155, −0.01155, 0.01155, 0.01155, −0.01155, −0.01154, 0.01154, −0.01154, −0.01154, 0.01153, 0.01153, −0.01153, 0.01153, 0.01153, −0.01153, 0.01152, −0.01152, 0.01152, 0.01152, 0.01152, −0.01152, 0.01152, −0.01152, −0.01151, −0.01151, 0.01151, 0.01151, −0.01151, −0.01151, 0.0115, −0.0115, −0.0115, 0.0115, 0.0115, −0.01149, −0.01149, 0.01149, 0.01149, 0.01149, −0.01149, −0.01148, −0.01148, −0.01148, 0.01148, −0.01148, 0.01148, 0.01148, −0.01148, −0.01148, −0.01148, −0.01148, −0.01148, −0.01148, −0.01147, 0.01147, −0.01147, 0.01147, −0.01147, −0.01147, 0.01147, 0.01147, −0.01146, −0.01146, −0.01146, −0.01146, 0.01146, 0.01146, −0.01146, −0.01145, 0.01145, −0.01145, −0.01145, −0.01145, −0.01145, 0.01144, −0.01144, −0.01144, 0.01144, 0.01144, −0.01144, 0.01144, 0.01144, 0.01143, −0.01143, 0.01143, −0.01143, 0.01142, 0.01142, 0.01142, 0.01142, −0.01142, −0.01142, −0.01142, −0.01142, −0.01142, −0.01142, 0.01142, −0.01141, 0.01141, −0.0114, 0.0114, −0.0114, −0.0114, −0.0114, 0.0114, 0.0114, −0.0114, −0.0114, 0.01139, −0.01139, −0.01139, 0.01139, 0.01139, 0.01139, 0.01139, 0.01139, 0.01139, −0.01138, 0.01138, 0.01138, −0.01138, 0.01138, 0.01138, 0.01138, 0.01137, 0.01137, 0.01137, 0.01137, 0.01137, 0.01136, −0.01136, 0.01136, 0.01136, −0.01136, −0.01136, 0.01136, 0.01136, 0.01136, 0.01135, 0.01135, 0.01135, 0.01135, 0.01135, 0.01134, −0.01134, −0.01134, 0.01134, 0.01134, −0.01134, −0.01134, 0.01134, 0.01134, −0.01133, 0.01133, 0.01133, −0.01133, 0.01133, 0.01133, 0.01133, −0.01132, 0.01132, −0.01132, 0.01132, −0.01132, 0.01132, 0.01132, −0.01132, 0.01132, 0.01132, −0.01132, 0.01131, 0.01131, −0.01131, 0.01131, −0.01131, −0.01131, −0.01131, −0.01131, −0.01131, −0.01131, −0.01131, 0.01131, 0.0113, −0.0113, −0.0113, −0.0113, −0.0113, −0.0113, −0.0113, 0.0113, 0.0113, 0.0113, 0.0113, −0.0113, −0.0113, 0.0113, −0.01129, −0.01129, 0.01129, 0.01129, −0.01129, −0.01129, 0.01129, 0.01129, −0.01129, 0.01129, −0.01128, −0.01128, −0.01128, 0.01128, 0.01127, 0.01127, 0.01127, −0.01127, −0.01127, 0.01127, 0.01127, −0.01127, −0.01126, 0.01126, 0.01126, −0.01126, 0.01126, 0.01126, −0.01126, −0.01126, 0.01126, 0.01126, 0.01125, −0.01125, −0.01125, 0.01125, −0.01125, −0.01125, 0.01125, 0.01124, 0.01124, −0.01124, 0.01124, 0.01124, 0.01124, −0.01124, 0.01124, 0.01123, −0.01123, 0.01123, 0.01123, 0.01123, −0.01123, 0.01122, −0.01122, −0.01122, −0.01122, 0.01122, −0.01122, 0.01121, 0.01121, −0.01121, −0.01121, −0.0112, 0.0112, −0.0112, −0.0112, 0.0112, 0.0112, 0.0112, 0.01119, 0.01119, 0.01119, 0.01119, −0.01119, 0.01119, 0.01119, −0.01119, −0.01119, 0.01119, 0.01119, 0.01118, −0.01118, −0.01118, −0.01118, −0.01118, 0.01117, −0.01117, −0.01117, −0.01117, −0.01117, −0.01116, −0.01116, −0.01116, −0.01116, −0.01116, −0.01116, 0.01115, −0.01115, −0.01115, −0.01115, −0.01115, −0.01115, −0.01114, 0.01114, −0.01113, −0.01113, 0.01113, −0.01113, −0.01113, 0.01113, −0.01113, −0.01113, −0.01113, −0.01113, 0.01113, 0.01112, −0.01112, −0.01112, 0.01112, 0.01111, −0.01111, −0.01111, 0.01111, 0.01111, −0.01111, 0.01111, 0.01111, −0.01111, 0.0111, −0.0111, −0.0111, 0.0111, −0.0111, 0.0111, 0.0111, −0.0111, 0.01109, 0.01109, −0.01109, 0.01108, 0.01108, −0.01108, 0.01108, −0.01108, 0.01108, 0.01107, −0.01107, −0.01107, 0.01107, 0.01107, 0.01107, 0.01106, −0.01106, 0.01106, 0.01106, −0.01106, −0.01106, 0.01106, 0.01106, 0.01105, 0.01105, −0.01105, −0.01105, −0.01104, 0.01104, 0.01104, 0.01104, 0.01104, −0.01104, 0.01103, −0.01103, 0.01103, 0.01103, 0.01103, 0.01103, 0.01103, 0.01103, −0.01103, 0.01102, −0.01102, −0.01102, 0.01102, 0.01102, 0.01102, −0.01102, 0.01101, −0.01101, 0.01101, −0.01101, −0.01101, 0.01101, 0.01101, 0.01101, −0.01101, −0.011, −0.011, 0.011, −0.011, 0.011, 0.011, −0.011, 0.011, −0.011, −0.01099, −0.01099, 0.01098, −0.01098, 0.01098, 0.01098, 0.01098, −0.01098, −0.01098, 0.01097, 0.01097, −0.01097, 0.01097, −0.01097, 0.01097, 0.01097, −0.01097, 0.01097, 0.01097, −0.01097, −0.01097, 0.01096, −0.01096, −0.01095, 0.01095, −0.01095, 0.01095, 0.01095, −0.01095, −0.01094, −0.01094, 0.01094, −0.01093, 0.01092, −0.01092, −0.01092, −0.01092, −0.01092, 0.01091, −0.01091, 0.01091, −0.01091, −0.01091, −0.01091, 0.0109, −0.0109, 0.0109, 0.0109, −0.0109, −0.0109, 0.01089, −0.01089, −0.01089, −0.01089, 0.01089, −0.01088, −0.01088, −0.01088, −0.01088, −0.01088, −0.01088, −0.01088, −0.01088, −0.01087, −0.01087, −0.01087, 0.01087, −0.01087, 0.01086, −0.01086, −0.01086, −0.01086, −0.01086, 0.01086, 0.01086, 0.01086, −0.01086, −0.01086, 0.01085, −0.01085, −0.01085, −0.01085, −0.01085, 0.01085, 0.01084, 0.01084, −0.01084, 0.01084, 0.01084, −0.01084, 0.01084, 0.01084, 0.01084, −0.01084, 0.01084, 0.01084, −0.01083, −0.01083, −0.01083, 0.01083, −0.01083, 0.01083, 0.01082, 0.01082, −0.01082, −0.01082, 0.01082, 0.01082, −0.01081, 0.01081, −0.01081, 0.01081, 0.01081, 0.01081, −0.01081, −0.0108, 0.0108, 0.0108, 0.0108, −0.0108, −0.0108, −0.0108, 0.0108, 0.0108, −0.0108, −0.0108, 0.0108, 0.01079, 0.01079, 0.01079, −0.01079, 0.01079, −0.01079, 0.01079, 0.01078, 0.01078, 0.01078, 0.01078, −0.01078, 0.01078, −0.01078, −0.01078, −0.01078, 0.01078, −0.01077, −0.01077, −0.01077, 0.01077, −0.01077, −0.01077, 0.01077, 0.01076, −0.01076, 0.01076, −0.01076, 0.01076, 0.01075, −0.01075, 0.01075, 0.01075, −0.01075, −0.01075, 0.01075, 0.01075, −0.01075, 0.01075, −0.01074, 0.01074, −0.01074, 0.01074, 0.01074, −0.01074, 0.01074, −0.01073, 0.01073, 0.01073, −0.01073, −0.01073, −0.01073, 0.01073, −0.01073, −0.01072, 0.01072, 0.01072, 0.01072, 0.01072, 0.01072, 0.01071, −0.01071, 0.01071, 0.01071, 0.01071, 0.0107, 0.0107, 0.0107, 0.0107, 0.0107, 0.01069, −0.01069, −0.01069, −0.01069, 0.01069, 0.01069, −0.01069, −0.01069, 0.01068, 0.01068, −0.01068, −0.01068, 0.01068, 0.01068, 0.01068, −0.01068, 0.01068, 0.01068, −0.01067, −0.01067, 0.01067, −0.01067, 0.01067, −0.01067, −0.01067, 0.01067, 0.01067, 0.01067, −0.01067, −0.01067, 0.01066, −0.01066, 0.01066, 0.01066, 0.01066, 0.01066, 0.01066, −0.01066, −0.01066, −0.01066, −0.01066, 0.01066, −0.01066, 0.01066, −0.01066, −0.01065, −0.01065, −0.01065, −0.01065, −0.01065, −0.01064, −0.01064, 0.01064, −0.01064, 0.01064, −0.01064, 0.01063, −0.01063, 0.01063, 0.01063, 0.01063, 0.01063, 0.01063, −0.01063, 0.01063, −0.01063, −0.01062, −0.01062, 0.01062, 0.01062, 0.01062, 0.01062, 0.01062, 0.01062, 0.01062, −0.01062, 0.01062, −0.01062, −0.01062, 0.01061, −0.01061, 0.01061, −0.01061, −0.01061, −0.01061, 0.0106, −0.0106, 0.0106, 0.0106, 0.01059, 0.01059, 0.01059, −0.01059, 0.01059, 0.01059, −0.01059, −0.01059, −0.01059, −0.01059, −0.01059, 0.01058, −0.01058, −0.01058, −0.01058, −0.01058, 0.01058, 0.01058, −0.01058, 0.01057, 0.01057, 0.01057, 0.01057, 0.01057, 0.01057, 0.01057, −0.01057, 0.01056, −0.01056, 0.01056, −0.01056, −0.01056, 0.01055, 0.01055, 0.01055, 0.01055, −0.01055, −0.01055, −0.01055, −0.01055, 0.01055, −0.01054, 0.01054, 0.01054, 0.01054, −0.01054, −0.01054, −0.01054, 0.01053, −0.01053, −0.01053, −0.01052, −0.01052, −0.01052, −0.01052, 0.01052, 0.01052, 0.01052, −0.01051, 0.01051, 0.01051, −0.01051, −0.01051, −0.01051, 0.01051, 0.0105, 0.0105, −0.0105, −0.0105, −0.0105, −0.0105, −0.0105, 0.01049, 0.01049, 0.01049, 0.01049, 0.01049, 0.01049, 0.01049, 0.01049, −0.01049, −0.01049, −0.01049, −0.01048, 0.01048, 0.01048, 0.01048, −0.01048, 0.01048, −0.01048, 0.01048, 0.01048, 0.01048, −0.01048, −0.01048, −0.01048, 0.01048, 0.01047, −0.01047, 0.01047, −0.01047, −0.01047, −0.01047, 0.01047, 0.01046, 0.01046, 0.01046, −0.01046, −0.01046, 0.01046, 0.01046, −0.01046, 0.01046, 0.01045, 0.01045, −0.01045, 0.01045, 0.01045, 0.01045, −0.01045, −0.01044, 0.01044, −0.01044, 0.01044, 0.01044, −0.01044, −0.01044, 0.01044, 0.01043, 0.01043, 0.01043, −0.01043, 0.01042, −0.01042, 0.01042, 0.01042, 0.01041, 0.01041, 0.01041, 0.01041, 0.01041, −0.01041, 0.01041, 0.01041, −0.01041, 0.01041, 0.0104, −0.0104, 0.0104, 0.0104, 0.01039, −0.01039, −0.01039, 0.01039, 0.01039, 0.01039, −0.01039, 0.01038, −0.01038, −0.01038, −0.01038, 0.01038, 0.01038, 0.01038, 0.01037, 0.01037, 0.01037, −0.01037, −0.01037, 0.01037, 0.01037, 0.01037, 0.01037, −0.01037, −0.01037, 0.01037, 0.01037, −0.01037, 0.01037, −0.01036, −0.01036, 0.01036, 0.01036, −0.01036, −0.01036, 0.01036, −0.01035, 0.01035, 0.01035, 0.01035, 0.01035, −0.01034, −0.01034, 0.01034, 0.01034, −0.01034, 0.01034, −0.01034, 0.01033, −0.01033, −0.01033, −0.01033, −0.01033, 0.01033, −0.01033, −0.01033, 0.01033, −0.01033, −0.01033, 0.01033, −0.01033, 0.01033, 0.01032, 0.01032, 0.01032, 0.01032, 0.01032, −0.01032, −0.01032, −0.01032, 0.01032, −0.01031, 0.01031, −0.01031, −0.01031, −0.01031, 0.01031, 0.01031, −0.01031, 0.01031, −0.01031, 0.01031, 0.0103, −0.0103, −0.0103, −0.0103, 0.0103, 0.0103, −0.0103, −0.0103, 0.0103, −0.0103, 0.0103, 0.0103, −0.0103, −0.01029, −0.01029, −0.01029, 0.01029, 0.01029, −0.01029, −0.01029, 0.01029, −0.01029, −0.01029, 0.01029, −0.01028, 0.01028, 0.01028, 0.01028, 0.01028, 0.01028, 0.01028, −0.01028, −0.01028, −0.01028, 0.01028, −0.01027, −0.01027, 0.01027, 0.01027, −0.01027, −0.01027, −0.01026, 0.01026, 0.01026, −0.01026, −0.01026, −0.01026, 0.01026, −0.01026, −0.01026, 0.01026, −0.01026, 0.01025, −0.01025, 0.01025, −0.01025, 0.01025, 0.01025, −0.01025, −0.01025, −0.01025, −0.01024, 0.01024, −0.01024, 0.01024, 0.01024, −0.01023, 0.01023, −0.01023, −0.01023, −0.01023, −0.01023, −0.01023, 0.01023, −0.01023, 0.01022, −0.01022, 0.01022, 0.01022, −0.01022, −0.01021, −0.01021, −0.01021, −0.01021, −0.01021, 0.01021, −0.01021, −0.0102, 0.0102, 0.0102, 0.0102, −0.0102, −0.0102, −0.0102, 0.0102, −0.0102, 0.0102, −0.0102, −0.0102, −0.0102, 0.01019, −0.01019, 0.01019, 0.01019, −0.01019, −0.01019, −0.01019, 0.01019, −0.01018, 0.01018, 0.01018, 0.01018, 0.01018, −0.01018, 0.01018, −0.01018, −0.01018, 0.01017, 0.01017, 0.01017, 0.01017, −0.01016, −0.01016, −0.01016, −0.01016, 0.01016, 0.01016, 0.01016, −0.01016, −0.01016, 0.01016, 0.01016, 0.01016, −0.01015, 0.01015, 0.01015, −0.01015, 0.01014, 0.01014, 0.01014, −0.01014, 0.01014, 0.01014, −0.01014, −0.01014, −0.01014, 0.01014, −0.01014, −0.01013, 0.01013, −0.01013, 0.01013, −0.01013, −0.01013, 0.01013, −0.01012, 0.01012, 0.01012, 0.01012, 0.01012, 0.01012, −0.01012, 0.01012, 0.01012, −0.01011, 0.01011, −0.01011, 0.01011, −0.01011, −0.01011, 0.01011, 0.01011, 0.01011, 0.0101, 0.0101, 0.0101, −0.0101, −0.0101, −0.0101, −0.0101, −0.01009, 0.01009, 0.01009, 0.01008, −0.01008, −0.01008, 0.01008, 0.01008, −0.01008, −0.01008, 0.01008, 0.01007, 0.01007, −0.01007, 0.01007, −0.01007, 0.01007, 0.01007, 0.01007, −0.01006, −0.01006, −0.01006, 0.01006, −0.01006, 0.01006, −0.01006, −0.01006, 0.01005, −0.01005, −0.01005, 0.01005, 0.01005, 0.01005, 0.01005, 0.01004, 0.01004, 0.01004, 0.01004, −0.01004, −0.01004, 0.01004, 0.01004, −0.01003, 0.01003, 0.01003, 0.01003, 0.01003, 0.01003, 0.01003, −0.01002, −0.01002, 0.01002, −0.01002, −0.01002, −0.01002, 0.01001, 0.01001, 0.01001, −0.01001, 0.01001, 0.01001, 0.01001, −0.01001, −0.01, 0.01, 0.01, 0.01, −0.01, −0.01, 0.01, −0.01, 0.01, −0.00999, 0.00999, −0.00999, 0.00999, −0.00999, 0.00999, 0.00999, −0.00999, 0.00999, 0.00999, −0.00998, 0.00998, −0.00998, −0.00998, 0.00998, −0.00998, 0.00998, −0.00998, 0.00998, 0.00998, 0.00997, −0.00997, 0.00997, 0.00997, 0.00997, −0.00997, −0.00997, 0.00997, 0.00997, −0.00997, −0.00997, 0.00996, 0.00996, −0.00996, 0.00996, 0.00996, −0.00996, 0.00996, −0.00996, −0.00996, 0.00996, −0.00996, −0.00996, 0.00995, 0.00995, −0.00995, 0.00995, −0.00995, −0.00995, −0.00995, 0.00995, 0.00995, 0.00995, −0.00995, −0.00995, 0.00995, 0.00995, 0.00994, −0.00994, 0.00994, 0.00994, −0.00994, −0.00994, 0.00993, −0.00993, 0.00993, 0.00993, −0.00992, 0.00992, 0.00992, 0.00992, 0.00992, −0.00992, 0.00992, −0.00991, 0.00991, −0.00991, 0.00991, −0.00991, −0.00991, −0.00991, −0.00991, 0.0099, −0.0099, 0.0099, 0.0099, −0.0099, −0.0099, −0.0099, −0.0099, −0.00989, 0.00989, 0.00989, 0.00989, −0.00989, 0.00989, 0.00989, −0.00989, 0.00989, −0.00989, −0.00989, 0.00988, −0.00988, −0.00988, −0.00988, 0.00988, 0.00988, −0.00988, 0.00988, −0.00987, −0.00987, 0.00987, 0.00987, −0.00987, 0.00987, −0.00987, 0.00987, 0.00987, 0.00987, 0.00987, 0.00987, 0.00987, 0.00986, −0.00986, −0.00986, 0.00985, 0.00985, 0.00985, 0.00985, −0.00985, −0.00985, −0.00984, 0.00984, −0.00984, −0.00984, −0.00984, 0.00984, 0.00983, −0.00983, 0.00983, 0.00983, −0.00983, 0.00982, 0.00982, −0.00982, −0.00982, 0.00982, 0.00981, −0.00981, 0.00981, 0.00981, −0.00981, 0.00981, −0.00981, −0.0098, −0.0098, 0.0098, −0.0098, 0.0098, 0.0098, 0.0098, −0.0098, 0.00979, −0.00979, −0.00979, −0.00979, 0.00979, 0.00979, 0.00979, 0.00978, −0.00978, 0.00978, −0.00978, 0.00978, −0.00978, −0.00978, −0.00977, 0.00977, 0.00977, 0.00977, 0.00977, −0.00977, 0.00977, −0.00977, −0.00976, −0.00976, −0.00976, −0.00976, 0.00976, 0.00976, 0.00976, 0.00976, 0.00976, 0.00976, −0.00975, −0.00975, 0.00975, −0.00975, 0.00975, −0.00975, 0.00975, −0.00975, −0.00975, 0.00975, −0.00974, −0.00974, −0.00974, −0.00974, −0.00974, 0.00974, −0.00974, 0.00974, 0.00974, 0.00973, −0.00973, 0.00973, 0.00973, 0.00973, 0.00973, −0.00973, 0.00973, −0.00973, −0.00973, 0.00973, 0.00972, 0.00972, −0.00972, 0.00972, −0.00972, −0.00972, 0.00972, −0.00972, −0.00972, 0.00972, 0.00972, 0.00972, −0.00972, −0.00972, −0.00971, 0.00971, 0.00971, 0.00971, 0.00971, 0.00971, −0.0097, 0.0097, 0.0097, 0.00969, −0.00969, 0.00969, 0.00969, −0.00969, −0.00969, −0.00969, −0.00969, −0.00969, 0.00969, 0.00969, 0.00968, −0.00968, −0.00968, −0.00968, −0.00968, 0.00967, −0.00967, 0.00967, −0.00967, 0.00967, 0.00967, −0.00967, 0.00967, 0.00966, −0.00966, 0.00966, 0.00966, −0.00966, 0.00966, −0.00966, 0.00966, −0.00966, 0.00966, 0.00965, −0.00965, 0.00965, 0.00965, −0.00965, 0.00965, 0.00965, −0.00965, 0.00964, −0.00964, 0.00964, 0.00964, 0.00964, −0.00964, −0.00964, 0.00964, 0.00964, 0.00964, −0.00963, 0.00963, 0.00963, 0.00963, −0.00963, −0.00963, 0.00962, −0.00962, 0.00962, 0.00962, −0.00961, 0.00961, −0.00961, 0.00961, 0.00961, −0.00961, −0.00961, −0.00961, 0.0096, −0.0096, −0.0096, 0.0096, 0.0096, 0.00959, 0.00959, 0.00959, −0.00959, 0.00959, 0.00959, 0.00959, 0.00959, 0.00959, 0.00959, 0.00959, −0.00959, −0.00959, −0.00958, −0.00958, 0.00958, 0.00958, −0.00958, −0.00958, 0.00958, −0.00958, −0.00958, −0.00958, −0.00958, 0.00958, 0.00958, −0.00957, 0.00957, 0.00957, −0.00957, −0.00957, −0.00956, 0.00956, −0.00956, −0.00956, 0.00956, 0.00956, −0.00955, −0.00955, −0.00955, 0.00955, −0.00955, 0.00955, 0.00955, −0.00955, 0.00955, −0.00955, 0.00954, −0.00954, 0.00954, 0.00954, 0.00954, −0.00954, 0.00954, 0.00954, −0.00954, 0.00953, −0.00953, 0.00953, 0.00953, 0.00953, −0.00953, 0.00953, −0.00953, −0.00953, −0.00953, 0.00953, −0.00953, −0.00953, −0.00953, 0.00953, 0.00953, 0.00952, 0.00952, 0.00952, 0.00951, 0.00951, −0.00951, −0.00951, −0.00951, 0.00951, 0.00951, −0.0095, −0.0095, 0.0095, 0.0095, −0.0095, 0.0095, 0.0095, −0.0095, −0.00949, −0.00949, −0.00949, 0.00949, −0.00949, 0.00949, −0.00949, −0.00948, 0.00948, −0.00948, −0.00948, −0.00948, 0.00948, 0.00948, 0.00947, −0.00947, −0.00947, −0.00947, −0.00947, −0.00947, 0.00947, 0.00947, −0.00947, −0.00947, −0.00946, −0.00946, 0.00946, 0.00946, 0.00946, 0.00946, 0.00946, −0.00946, 0.00946, −0.00946, 0.00946, 0.00946, 0.00946, 0.00945, 0.00945, 0.00945, −0.00945, 0.00945, −0.00945, −0.00945, −0.00945, 0.00945, −0.00945, −0.00945, −0.00944, 0.00944, −0.00944, 0.00944, 0.00944, −0.00944, 0.00944, 0.00944, 0.00944, 0.00944, 0.00944, −0.00943, 0.00943, 0.00943, −0.00943, 0.00943, −0.00943, −0.00943, 0.00943, 0.00943, 0.00943, −0.00943, −0.00942, 0.00942, 0.00942, −0.00942, 0.00941, 0.00941, 0.00941, −0.00941, −0.00941, 0.0094, 0.0094, 0.0094, −0.0094, −0.0094, −0.0094, −0.0094, −0.00939, −0.00939, 0.00939, 0.00939, 0.00939, 0.00939, −0.00938, 0.00938, 0.00938, −0.00938, −0.00938, −0.00938, 0.00938, −0.00938, 0.00938, −0.00938, 0.00937, 0.00937, −0.00937, −0.00937, 0.00937, 0.00937, −0.00937, −0.00937, 0.00936, −0.00936, 0.00936, 0.00935, 0.00935, −0.00935, 0.00935, 0.00935, −0.00935, 0.00935, 0.00935, 0.00934, −0.00934, −0.00934, 0.00934, 0.00934, −0.00934, 0.00934, −0.00933, −0.00933, −0.00933, 0.00933, −0.00933, 0.00933, 0.00933, 0.00933, 0.00933, 0.00932, 0.00932, 0.00932, −0.00932, 0.00932, −0.00932, 0.00932, 0.00932, −0.00931, −0.00931, 0.00931, 0.00931, −0.00931, 0.0093, 0.0093, 0.0093, −0.0093, −0.0093, −0.0093, −0.0093, −0.0093, −0.0093, 0.0093, 0.0093, 0.0093, 0.00929, −0.00929, 0.00929, 0.00929, 0.00929, 0.00929, 0.00929, −0.00928, −0.00928, 0.00928, −0.00928, −0.00928, 0.00928, −0.00927, 0.00927, 0.00927, 0.00927, 0.00927, −0.00927, −0.00927, −0.00926, 0.00926, 0.00926, 0.00925, −0.00925, −0.00925, −0.00925, 0.00925, 0.00925, −0.00924, 0.00924, −0.00924, −0.00924, 0.00924, −0.00924, −0.00924, −0.00924, −0.00924, 0.00924, 0.00924, 0.00924, 0.00924, 0.00923, −0.00923, −0.00923, 0.00923, −0.00923, −0.00923, 0.00923, −0.00923, 0.00922, 0.00922, 0.00922, 0.00922, 0.00922, 0.00922, −0.00921, 0.00921, 0.00921, −0.00921, −0.00921, 0.00921, −0.00921, 0.00921, −0.00921, −0.00921, 0.0092, 0.0092, 0.0092, 0.0092, −0.0092, 0.0092, 0.00919, 0.00919, 0.00919, −0.00919, 0.00919, −0.00919, −0.00919, 0.00919, 0.00919, 0.00919, 0.00918, 0.00918, −0.00918, −0.00918, 0.00918, 0.00918, −0.00918, −0.00918, −0.00917, −0.00917, −0.00917, 0.00917, −0.00917, 0.00917, 0.00917, 0.00917, 0.00917, 0.00917, 0.00917, 0.00917, −0.00917, −0.00916, 0.00916, 0.00916, −0.00916, −0.00916, −0.00916, −0.00916, 0.00916, 0.00916, −0.00915, 0.00915, −0.00915, 0.00915, 0.00915, 0.00915, −0.00915, −0.00915, −0.00914, 0.00914, 0.00914, −0.00914, −0.00914, 0.00914, 0.00914, 0.00913, 0.00913, 0.00913, 0.00913, 0.00913, −0.00913, −0.00913, −0.00913, −0.00912, 0.00912, −0.00912, 0.00912, 0.00912, −0.00912, −0.00912, 0.00911, −0.00911, −0.00911, 0.0091, −0.0091, −0.0091, 0.0091, −0.0091, 0.0091, 0.00909, 0.00909, 0.00909, −0.00909, −0.00909, −0.00908, −0.00908, 0.00908, −0.00908, −0.00908, −0.00908, −0.00907, −0.00907, −0.00907, −0.00907, 0.00907, −0.00907, −0.00907, 0.00907, 0.00906, 0.00906, 0.00906, 0.00906, 0.00906, −0.00906, 0.00906, 0.00906, 0.00906, 0.00906, −0.00906, 0.00906, −0.00906, 0.00906, 0.00906, −0.00906, −0.00905, 0.00905, 0.00905, 0.00905, 0.00905, −0.00905, −0.00905, −0.00905, 0.00905, 0.00904, 0.00904, −0.00904, 0.00904, 0.00904, 0.00904, −0.00904, 0.00904, 0.00904, 0.00903, −0.00903, 0.00903, 0.00903, −0.00903, 0.00903, 0.00903, −0.00903, 0.00903, 0.00903, 0.00903, −0.00902, 0.00902, 0.00902, −0.00902, 0.00902, −0.00902, −0.00902, −0.00902, −0.00902, −0.00902, 0.00901, 0.00901, 0.00901, −0.00901, −0.00901, 0.00901, 0.00901, 0.00901, 0.00901, −0.00901, −0.00901, 0.00901, 0.00901, −0.009, 0.009, −0.009, 0.009, −0.009, 0.009, −0.009, 0.009, 0.00899, 0.00899, −0.00899, −0.00899, 0.00899, −0.00899, −0.00899, −0.00898, 0.00898, 0.00898, −0.00898, 0.00898, 0.00897, −0.00897, 0.00897, −0.00897, −0.00897, −0.00897, −0.00897, 0.00897, −0.00896, 0.00896, 0.00896, −0.00896, 0.00895, −0.00895, 0.00895, −0.00895, −0.00895, −0.00895, −0.00895, −0.00895, −0.00895, 0.00895, 0.00895, 0.00895, 0.00895, 0.00895, −0.00894, −0.00894, 0.00894, 0.00894, −0.00894, 0.00894, 0.00894, −0.00894, −0.00894, −0.00893, 0.00893, 0.00893, −0.00893, −0.00892, −0.00892, 0.00892, −0.00892, −0.00892, 0.00892, −0.00892, 0.00891, −0.00891, −0.00891, −0.00891, −0.00891, −0.00891, 0.00891, −0.00891, 0.00891, −0.00891, −0.0089, 0.0089, 0.0089, 0.0089, 0.0089, −0.0089, −0.0089, −0.0089, −0.0089, −0.0089, 0.00889, −0.00889, 0.00889, −0.00889, 0.00889, −0.00889, 0.00889, −0.00889, −0.00889, −0.00889, −0.00889, −0.00889, 0.00888, −0.00888, 0.00888, −0.00888, 0.00888, 0.00888, −0.00888, 0.00888, −0.00888, −0.00887, 0.00887, 0.00887, 0.00887, −0.00887, 0.00887, −0.00887, −0.00887, 0.00887, −0.00887, 0.00887, 0.00887, −0.00886, 0.00886, 0.00886, −0.00886, 0.00886, −0.00886, 0.00886, 0.00886, 0.00885, −0.00885, 0.00885, −0.00885, −0.00885, 0.00885, −0.00884, −0.00884, 0.00884, 0.00884, −0.00884, −0.00884, −0.00884, 0.00884, −0.00884, 0.00883, 0.00883, −0.00883, 0.00883, 0.00883, −0.00883, −0.00883, −0.00883, 0.00883, 0.00883, −0.00883, −0.00883, 0.00882, 0.00882, 0.00882, 0.00882, −0.00882, −0.00882, 0.00882, −0.00882, −0.00882, 0.00881, −0.00881, −0.00881, −0.00881, −0.00881, 0.00881, 0.00881, 0.00881, −0.0088, 0.0088, 0.0088, −0.0088, −0.0088, −0.0088, 0.0088, 0.0088, 0.00879, 0.00879, −0.00879, −0.00879, −0.00879, −0.00879, 0.00879, −0.00879, 0.00878, 0.00878, −0.00878, 0.00878, −0.00878, 0.00878, −0.00878, −0.00878, −0.00877, −0.00877, 0.00877, 0.00877, −0.00877, −0.00877, −0.00877, 0.00877, −0.00877, −0.00876, −0.00876, −0.00876, 0.00876, 0.00876, 0.00876, 0.00876, −0.00876, −0.00875, −0.00875, −0.00875, −0.00875, 0.00875, 0.00875, −0.00875, 0.00875, 0.00874, −0.00874, 0.00874, 0.00874, 0.00874, 0.00874, −0.00874, −0.00874, 0.00873, 0.00873, −0.00873, −0.00873, 0.00873, 0.00873, −0.00873, −0.00873, 0.00873, −0.00872, 0.00872, 0.00872, 0.00872, 0.00872, −0.00872, 0.00872, −0.00871, −0.00871, −0.00871, −0.00871, −0.0087, 0.0087, −0.0087, 0.0087, 0.0087, 0.0087, −0.0087, 0.0087, −0.0087, −0.0087, 0.00869, 0.00869, −0.00869, −0.00869, 0.00869, 0.00869, −0.00869, 0.00868, 0.00868, 0.00868, 0.00868, −0.00868, 0.00868, 0.00867, 0.00867, −0.00867, −0.00867, −0.00867, −0.00867, −0.00867, −0.00867, 0.00867, −0.00867, 0.00867, 0.00867, −0.00867, −0.00867, 0.00866, 0.00866, −0.00866, −0.00866, −0.00866, 0.00866, 0.00866, −0.00866, 0.00865, 0.00865, 0.00865, 0.00865, −0.00865, −0.00865, −0.00864, 0.00864, 0.00864, −0.00864, −0.00864, −0.00864, 0.00864, −0.00864, −0.00864, 0.00864, 0.00864, 0.00864, −0.00863, 0.00863, 0.00862, 0.00862, −0.00862, −0.00862, 0.00862, 0.00862, 0.00862, 0.00861, 0.00861, −0.00861, 0.00861, 0.00861, 0.00861, 0.00861, 0.00861, −0.00861, −0.00861, −0.0086, −0.0086, 0.0086, −0.0086, 0.0086, 0.0086, 0.0086, −0.0086, 0.0086, −0.0086, −0.00859, 0.00859, −0.00859, 0.00859, 0.00859, −0.00859, −0.00858, 0.00858, 0.00858, 0.00858, 0.00858, 0.00858, 0.00858, −0.00858, 0.00858, −0.00858, 0.00857, 0.00857, 0.00857, −0.00857, 0.00857, −0.00857, −0.00857, −0.00857, −0.00857, −0.00857, 0.00857, −0.00857, −0.00857, 0.00857, 0.00857, −0.00856, 0.00856, 0.00856, −0.00856, 0.00856, 0.00856, 0.00856, −0.00855, 0.00855, 0.00855, −0.00855, 0.00855, −0.00855, 0.00855, −0.00855, 0.00855, 0.00854, −0.00854, −0.00854, 0.00854, 0.00854, 0.00854, −0.00854, −0.00854, 0.00854, −0.00854, −0.00854, 0.00854, 0.00853, −0.00853, −0.00853, −0.00853, 0.00853, −0.00853, 0.00852, −0.00852, −0.00852, 0.00852, 0.00852, −0.00852, −0.00852, 0.00852, −0.00852, 0.00852, −0.00852, −0.00852, −0.00851, 0.00851, 0.00851, 0.00851, −0.00851, 0.00851, −0.00851, −0.00851, 0.00851, −0.0085, −0.0085, −0.0085, 0.0085, 0.0085, −0.0085, −0.0085, −0.0085, −0.00849, −0.00849, 0.00849, 0.00849, 0.00849, 0.00849, 0.00849, −0.00849, −0.00849, −0.00848, −0.00848, 0.00848, 0.00848, 0.00848, −0.00848, −0.00848, −0.00848, −0.00848, −0.00848, −0.00848, 0.00848, −0.00848, 0.00847, 0.00847, −0.00847, 0.00847, 0.00847, 0.00847, −0.00847, −0.00847, −0.00846, −0.00846, −0.00846, 0.00846, −0.00846, −0.00845, −0.00845, 0.00845, 0.00845, 0.00845, 0.00845, 0.00845, 0.00845, 0.00844, −0.00844, −0.00844, −0.00844, 0.00843, −0.00843, −0.00843, 0.00843, −0.00843, 0.00843, −0.00843, 0.00843, 0.00843, −0.00842, 0.00842, 0.00842, −0.00842, 0.00842, 0.00842, −0.00842, 0.00842, −0.00842, 0.00842, 0.00841, −0.00841, −0.00841, 0.00841, 0.00841, −0.00841, −0.00841, −0.0084, −0.0084, −0.0084, −0.0084, 0.0084, 0.00839, −0.00839, −0.00839, −0.00839, 0.00839, −0.00839, −0.00839, −0.00839, −0.00839, −0.00839, −0.00838, 0.00838, 0.00838, 0.00838, −0.00838, −0.00838, 0.00838, 0.00838, 0.00838, 0.00838, −0.00838, 0.00838, 0.00837, −0.00837, 0.00837, −0.00837, −0.00837, −0.00837, −0.00837, −0.00837, 0.00836, −0.00836, 0.00836, 0.00836, 0.00836, −0.00836, −0.00835, −0.00835, −0.00835, 0.00835, 0.00835, 0.00835, 0.00835, 0.00834, −0.00834, −0.00834, 0.00834, 0.00834, 0.00834, 0.00834, 0.00834, 0.00834, −0.00834, −0.00834, −0.00834, −0.00834, 0.00833, −0.00833, −0.00833, 0.00833, 0.00833, −0.00833, −0.00833, 0.00833, 0.00833, −0.00833, 0.00833, −0.00833, −0.00832, 0.00832, −0.00832, 0.00832, 0.00832, −0.00832, −0.00831, 0.00831, 0.00831, −0.0083, −0.0083, 0.0083, 0.0083, 0.0083, −0.00829, 0.00829, −0.00829, 0.00829, 0.00829, −0.00829, 0.00829, 0.00828, 0.00828, −0.00828, −0.00828, −0.00828, −0.00828, −0.00828, 0.00827, 0.00827, 0.00827, 0.00827, −0.00827, −0.00827, 0.00827, 0.00827, 0.00826, −0.00826, −0.00826, −0.00826, 0.00826, 0.00826, −0.00826, −0.00826, −0.00825, −0.00825, −0.00825, 0.00825, 0.00825, −0.00825, 0.00825, 0.00825, 0.00825, 0.00825, 0.00825, 0.00825, −0.00825, 0.00824, −0.00824, 0.00824, 0.00824, 0.00824, 0.00823, 0.00823, −0.00823, −0.00823, 0.00823, −0.00823, −0.00823, 0.00822, −0.00822, −0.00822, −0.00822, −0.00822, 0.00822, −0.00822, 0.00822, −0.00822, −0.00822, 0.00821, 0.00821, −0.00821, 0.00821, 0.00821, −0.00821, 0.00821, 0.00821, 0.00821, 0.00821, 0.00821, −0.00821, −0.00821, 0.0082, −0.0082, 0.0082, 0.0082, 0.0082, −0.0082, −0.0082, 0.0082, 0.00819, 0.00819, −0.00819, 0.00819, 0.00819, −0.00819, 0.00819, −0.00818, −0.00818, −0.00818, −0.00818, 0.00818, 0.00818, −0.00818, 0.00818, 0.00817, −0.00817, 0.00817, 0.00817, 0.00817, 0.00817, −0.00817, −0.00817, −0.00816, 0.00816, 0.00816, 0.00816, −0.00816, 0.00816, −0.00816, 0.00816, 0.00816, −0.00815, −0.00815, 0.00815, −0.00815, 0.00815, −0.00815, 0.00815, −0.00814, 0.00814, −0.00814, −0.00814, 0.00814, 0.00814, −0.00814, 0.00814, −0.00814, 0.00814, 0.00813, −0.00813, −0.00813, −0.00813, −0.00812, −0.00812, −0.00812, −0.00812, −0.00812, 0.00812, −0.00812, 0.00812, −0.00812, −0.00812, −0.00811, 0.00811, 0.00811, 0.00811, 0.00811, 0.00811, 0.00811, 0.00811, −0.00811, −0.00811, 0.0081, 0.0081, −0.0081, 0.0081, 0.0081, −0.00809, 0.00809, −0.00809, −0.00809, −0.00809, 0.00809, −0.00808, −0.00808, 0.00808, 0.00808, 0.00808, 0.00808, 0.00808, −0.00807, 0.00807, −0.00807, −0.00807, −0.00807, 0.00807, −0.00807, 0.00806, 0.00806, 0.00806, −0.00806, −0.00806, 0.00805, 0.00805, 0.00805, 0.00805, 0.00805, −0.00805, −0.00805, −0.00805, 0.00805, 0.00804, −0.00804, −0.00804, −0.00804, −0.00804, −0.00804, 0.00804, −0.00804, 0.00804, −0.00804, −0.00804, −0.00804, −0.00804, 0.00803, 0.00803, −0.00803, −0.00803, 0.00803, −0.00803, 0.00803, −0.00803, −0.00802, 0.00802, 0.00802, −0.00802, −0.00802, 0.00802, 0.00802, 0.00802, −0.00802, 0.00801, −0.00801, 0.00801, 0.00801, 0.008, −0.008, 0.008, 0.008, 0.008, 0.008, −0.008, −0.008, 0.008, −0.008, −0.008, −0.00799, 0.00799, 0.00799, −0.00799, −0.00799, 0.00799, −0.00799, 0.00799, 0.00799, −0.00798, 0.00798, 0.00798, 0.00798, 0.00798, −0.00797, −0.00797, −0.00797, −0.00797, −0.00797, −0.00797, 0.00797, −0.00797, 0.00797, 0.00797, −0.00797, −0.00796, 0.00796, 0.00796, 0.00796, 0.00796, 0.00796, −0.00795, 0.00795, −0.00795, −0.00795, −0.00795, −0.00795, 0.00795, 0.00794, 0.00794, −0.00794, −0.00794, −0.00794, 0.00794, −0.00794, 0.00793, −0.00793, −0.00793, 0.00793, −0.00793, 0.00793, 0.00793, −0.00793, 0.00793, −0.00793, 0.00793, 0.00793, −0.00792, 0.00792, 0.00792, 0.00792, −0.00792, 0.00792, −0.00792, −0.00792, −0.00791, 0.00791, 0.00791, −0.00791, −0.00791, 0.00791, 0.00791, 0.00791, 0.00791, −0.0079, 0.0079, −0.0079, −0.0079, −0.0079, 0.0079, 0.0079, 0.0079, −0.00789, 0.00789, 0.00789, −0.00789, 0.00789, −0.00789, −0.00789, −0.00788, 0.00788, 0.00788, 0.00788, 0.00788, −0.00788, 0.00787, 0.00787, −0.00787, 0.00787, −0.00787, −0.00787, 0.00787, 0.00787, −0.00786, −0.00786, −0.00786, −0.00786, 0.00786, 0.00786, 0.00786, 0.00786, 0.00785, −0.00785, −0.00785, −0.00785, 0.00785, −0.00785, −0.00784, −0.00784, −0.00784, −0.00784, −0.00784, 0.00784, −0.00784, 0.00784, −0.00784, −0.00784, −0.00784, −0.00784, 0.00783, 0.00783, −0.00783, 0.00783, −0.00783, 0.00783, 0.00783, −0.00783, 0.00783, 0.00783, 0.00783, −0.00782, −0.00782, −0.00782, 0.00782, −0.00782, −0.00782, −0.00782, 0.00782, 0.00781, 0.00781, −0.00781, 0.00781, 0.00781, 0.00781, 0.00781, 0.00781, −0.00781, 0.00781, −0.00781, −0.0078, −0.0078, 0.0078, −0.0078, −0.0078, −0.0078, −0.00779, 0.00779, 0.00779, 0.00779, −0.00779, 0.00779, 0.00779, −0.00779, −0.00779, −0.00779, 0.00779, −0.00778, −0.00778, 0.00778, 0.00778, 0.00778, 0.00777, −0.00777, −0.00777, 0.00777, −0.00777, −0.00777, −0.00777, 0.00777, −0.00776, 0.00776, 0.00776, −0.00776, −0.00776, −0.00776, −0.00775, 0.00775, 0.00775, 0.00775, 0.00775, −0.00775, 0.00774, 0.00774, −0.00774, 0.00774, −0.00774, −0.00774, −0.00774, −0.00774, −0.00774, 0.00774, 0.00774, 0.00774, 0.00774, −0.00774, −0.00774, −0.00774, 0.00773, −0.00773, −0.00773, −0.00773, −0.00772, −0.00772, −0.00772, 0.00772, 0.00772, 0.00772, 0.00772, 0.00772, −0.00772, −0.00771, −0.00771, 0.00771, −0.00771, −0.00771, −0.00771, 0.00771, −0.00771, −0.00771, −0.00771, −0.0077, 0.0077, 0.0077, −0.00769, −0.00769, 0.00769, −0.00769, −0.00769, −0.00769, 0.00769, −0.00769, 0.00768, 0.00768, −0.00768, −0.00768, 0.00768, 0.00768, −0.00768, 0.00768, 0.00768, 0.00768, −0.00768, 0.00768, −0.00768, 0.00767, −0.00767, 0.00767, 0.00767, −0.00767, 0.00767, 0.00767, 0.00767, −0.00767, −0.00766, −0.00766, −0.00766, 0.00766, −0.00766, 0.00766, 0.00766, 0.00766, 0.00766, −0.00765, 0.00765, 0.00765, −0.00765, −0.00765, −0.00765, 0.00765, 0.00765, −0.00764, −0.00764, −0.00764, 0.00764, −0.00764, 0.00764, −0.00764, −0.00763, 0.00763, −0.00763, −0.00763, 0.00763, 0.00763, 0.00763, −0.00763, 0.00763, 0.00763, −0.00763, −0.00763, −0.00762, 0.00762, 0.00762, 0.00762, −0.00762, 0.00762, −0.00762, −0.00762, −0.00762, 0.00762, −0.00762, −0.00762, 0.00762, 0.00762, −0.00762, 0.00761, −0.00761, 0.00761, −0.0076, −0.0076, −0.0076, 0.0076, 0.0076, 0.0076, 0.0076, −0.0076, −0.0076, −0.0076, 0.00759, −0.00759, 0.00759, 0.00759, 0.00759, 0.00759, 0.00759, 0.00759, 0.00759, 0.00758, −0.00758, −0.00758, −0.00758, −0.00758, −0.00758, −0.00758, −0.00758, −0.00758, 0.00758, 0.00758, −0.00758, 0.00758, 0.00758, 0.00758, −0.00758, 0.00757, −0.00757, −0.00757, −0.00757, 0.00757, −0.00757, 0.00757, 0.00757, 0.00757, 0.00757, 0.00756, 0.00756, −0.00756, 0.00756, −0.00756, 0.00756, −0.00756, 0.00756, −0.00756, −0.00756, −0.00756, 0.00755, 0.00755, 0.00755, −0.00755, 0.00755, 0.00755, −0.00755, −0.00755, 0.00755, 0.00755, −0.00755, 0.00755, 0.00754, −0.00754, 0.00754, −0.00754, −0.00754, 0.00754, −0.00754, −0.00754, 0.00754, −0.00754, 0.00754, 0.00754, 0.00754, 0.00753, 0.00753, −0.00753, −0.00753, 0.00752, −0.00752, −0.00752, 0.00752, 0.00752, −0.00752, −0.00752, 0.00751, 0.00751, 0.00751, 0.00751, −0.00751, 0.00751, −0.00751, 0.00751, −0.0075, 0.0075, 0.0075, −0.0075, −0.0075, 0.0075, 0.0075, −0.0075, 0.0075, −0.00749, 0.00749, −0.00749, 0.00749, −0.00749, −0.00749, 0.00749, −0.00749, 0.00748, 0.00748, 0.00748, −0.00748, 0.00748, 0.00747, 0.00747, 0.00747, 0.00747, 0.00747, −0.00747, 0.00747, 0.00747, 0.00747, −0.00747, 0.00746, 0.00746, −0.00746, 0.00746, −0.00746, −0.00746, 0.00745, −0.00745, 0.00745, 0.00745, −0.00745, 0.00745, −0.00745, −0.00745, −0.00745, −0.00745, −0.00745, −0.00745, 0.00745, 0.00745, −0.00745, 0.00745, 0.00745, 0.00745, 0.00745, 0.00744, −0.00744, −0.00744, 0.00744, 0.00744, −0.00744, 0.00744, 0.00744, 0.00744, −0.00744, −0.00744, 0.00744, 0.00744, 0.00743, −0.00743, 0.00743, 0.00743, 0.00743, 0.00743, 0.00743, 0.00743, −0.00743, −0.00743, 0.00743, 0.00742, −0.00742, 0.00742, −0.00742, 0.00742, −0.00742, −0.00742, −0.00742, 0.00742, 0.00742, 0.00742, 0.00742, −0.00741, −0.00741, 0.00741, 0.00741, −0.00741, −0.00741, −0.00741, −0.00741, 0.00741, 0.00741, −0.00741, 0.0074, −0.0074, −0.0074, −0.00739, −0.00739, 0.00739, −0.00739, 0.00739, −0.00739, 0.00739, 0.00739, −0.00738, −0.00738, −0.00738, 0.00738, 0.00738, −0.00738, −0.00738, −0.00738, −0.00738, −0.00738, −0.00737, −0.00737, −0.00737, −0.00737, −0.00737, −0.00737, −0.00737, 0.00737, 0.00737, 0.00736, −0.00736, 0.00736, 0.00736, 0.00736, 0.00736, 0.00736, 0.00736, −0.00736, −0.00736, −0.00735, 0.00735, 0.00735, −0.00735, 0.00735, 0.00735, 0.00735, −0.00735, −0.00735, −0.00735, 0.00735, −0.00735, −0.00734, −0.00734, −0.00734, −0.00734, 0.00734, 0.00734, −0.00734, −0.00734, 0.00734, 0.00734, −0.00734, −0.00734, 0.00734, −0.00734, −0.00734, −0.00734, −0.00733, −0.00733, 0.00733, −0.00733, 0.00733, 0.00733, 0.00733, 0.00733, −0.00733, −0.00733, 0.00733, 0.00733, 0.00732, 0.00732, −0.00732, 0.00732, −0.00732, −0.00732, 0.00732, −0.00732, −0.00732, −0.00732, 0.00731, −0.00731, 0.00731, 0.00731, 0.00731, 0.00731, −0.00731, −0.0073, 0.0073, 0.0073, −0.00729, −0.00729, −0.00729, −0.00729, 0.00729, 0.00728, 0.00728, −0.00728, 0.00728, 0.00728, −0.00728, 0.00728, 0.00728, −0.00727, 0.00727, −0.00727, 0.00727, 0.00727, 0.00727, −0.00727, −0.00727, 0.00726, 0.00726, 0.00726, −0.00726, 0.00726, 0.00726, −0.00726, 0.00726, −0.00725, 0.00725, 0.00725, −0.00725, 0.00725, −0.00725, 0.00725, −0.00725, −0.00725, 0.00724, 0.00724, −0.00724, 0.00724, −0.00724, 0.00724, 0.00724, 0.00724, −0.00723, −0.00723, 0.00723, 0.00723, 0.00723, 0.00723, 0.00723, −0.00723, −0.00723, −0.00723, 0.00723, −0.00722, 0.00722, 0.00722, 0.00722, −0.00722, −0.00722, 0.00721, −0.00721, −0.00721, 0.00721, 0.00721, −0.00721, 0.00721, −0.00721, −0.00721, −0.00721, −0.00721, 0.0072, 0.0072, −0.0072, 0.0072, −0.0072, −0.0072, 0.0072, 0.00719, 0.00719, −0.00719, 0.00719, −0.00718, −0.00718, 0.00718, 0.00718, −0.00718, 0.00718, 0.00718, −0.00718, 0.00718, 0.00718, 0.00718, −0.00717, −0.00717, 0.00717, 0.00717, −0.00717, 0.00716, 0.00716, 0.00716, −0.00716, 0.00716, −0.00716, −0.00716, −0.00716, 0.00716, 0.00716, −0.00716, 0.00716, −0.00715, −0.00715, 0.00715, 0.00715, −0.00715, 0.00715, −0.00715, 0.00715, −0.00715, −0.00715, −0.00715, 0.00715, −0.00714, 0.00714, −0.00714, −0.00714, 0.00714, −0.00714, −0.00714, 0.00713, 0.00713, 0.00713, 0.00713, 0.00713, −0.00712, −0.00712, −0.00712, −0.00712, −0.00712, 0.00712, −0.00712, 0.00712, 0.00711, −0.00711, 0.00711, −0.00711, −0.00711, −0.00711, −0.00711, −0.00711, 0.0071, −0.0071, −0.0071, 0.0071, 0.0071, 0.0071, −0.0071, −0.0071, −0.00709, 0.00709, 0.00709, 0.00709, −0.00709, −0.00709, 0.00709, 0.00709, −0.00709, 0.00708, −0.00708, −0.00708, −0.00708, 0.00708, 0.00708, −0.00708, −0.00708, 0.00708, −0.00708, 0.00708, −0.00708, −0.00708, 0.00707, 0.00707, 0.00707, 0.00707, 0.00707, 0.00707, 0.00707, −0.00707, −0.00706, −0.00706, 0.00706, 0.00706, 0.00706, −0.00706, 0.00706, 0.00706, 0.00705, −0.00705, −0.00705, 0.00705, 0.00705, −0.00705, 0.00705, −0.00705, −0.00705, 0.00704, 0.00704, 0.00704, 0.00704, −0.00704, 0.00704, 0.00703, 0.00703, 0.00703, −0.00703, −0.00703, 0.00703, 0.00703, 0.00703, −0.00703, −0.00703, 0.00703, 0.00703, 0.00703, 0.00703, 0.00703, −0.00703, −0.00703, 0.00703, 0.00702, −0.00702, −0.00702, −0.00702, −0.00702, −0.00702, −0.00701, 0.00701, −0.00701, −0.00701, 0.00701, 0.00701, −0.00701, −0.00701, 0.007, 0.007, −0.007, 0.007, 0.007, 0.007, −0.007, −0.007, 0.007, −0.007, 0.007, 0.00699, −0.00699, −0.00699, −0.00699, 0.00699, −0.00699, 0.00699, −0.00699, −0.00699, 0.00699, −0.00699, 0.00699, 0.00698, −0.00698, −0.00698, −0.00698, −0.00697, −0.00697, 0.00697, −0.00697, 0.00697, −0.00697, −0.00697, −0.00697, −0.00697, −0.00697, −0.00697, 0.00697, 0.00697, −0.00697, 0.00697, 0.00697, −0.00697, 0.00697, 0.00696, 0.00696, 0.00696, 0.00696, 0.00696, −0.00695, 0.00695, −0.00695, −0.00695, 0.00695, 0.00695, −0.00695, 0.00695, −0.00695, 0.00695, 0.00694, 0.00694, 0.00694, 0.00694, 0.00694, −0.00694, 0.00694, −0.00694, −0.00694, 0.00694, −0.00693, −0.00693, −0.00693, −0.00693, 0.00693, −0.00693, 0.00693, 0.00693, 0.00692, 0.00692, 0.00692, −0.00692, −0.00692, 0.00692, −0.00692, 0.00692, 0.00692, 0.00692, 0.00691, −0.00691, 0.00691, 0.00691, −0.00691, −0.00691, 0.00691, −0.00691, −0.00691, −0.00691, −0.00691, 0.00691, 0.00691, −0.00691, −0.00691, −0.0069, −0.0069, −0.0069, −0.0069, −0.0069, 0.0069, 0.0069, 0.00689, 0.00689, 0.00689, −0.00689, −0.00689, −0.00689, −0.00689, −0.00689, 0.00689, −0.00689, −0.00689, 0.00688, −0.00688, −0.00688, −0.00688, −0.00688, 0.00688, −0.00688, 0.00688, −0.00688, 0.00688, −0.00688, 0.00688, 0.00688, −0.00688, −0.00688, 0.00688, 0.00687, −0.00687, −0.00687, −0.00687, 0.00687, −0.00687, 0.00687, −0.00687, 0.00687, 0.00687, −0.00687, −0.00686, −0.00686, 0.00686, 0.00686, 0.00686, 0.00686, −0.00686, −0.00686, 0.00686, 0.00685, −0.00685, 0.00685, −0.00685, −0.00685, 0.00685, 0.00685, −0.00685, −0.00685, −0.00685, −0.00684, 0.00684, 0.00684, −0.00684, 0.00684, 0.00684, 0.00684, 0.00683, 0.00683, 0.00683, −0.00683, 0.00683, −0.00683, −0.00683, 0.00683, 0.00683, −0.00683, −0.00683, 0.00683, 0.00682, 0.00682, −0.00682, 0.00682, 0.00682, −0.00682, −0.00682, 0.00682, −0.00682, 0.00682, −0.00681, −0.00681, −0.00681, 0.00681, −0.00681, 0.00681, 0.00681, 0.00681, −0.00681, 0.0068, −0.0068, 0.0068, 0.0068, 0.00679, 0.00679, 0.00679, −0.00679, 0.00679, −0.00679, −0.00679, −0.00679, 0.00679, −0.00679, 0.00679, 0.00678, −0.00678, −0.00678, 0.00678, −0.00678, 0.00678, 0.00678, 0.00678, −0.00677, 0.00677, −0.00677, −0.00677, 0.00677, 0.00677, 0.00677, 0.00677, 0.00677, 0.00676, −0.00676, −0.00676, 0.00676, 0.00676, −0.00676, −0.00676, −0.00676, −0.00676, −0.00675, −0.00675, 0.00675, 0.00675, −0.00675, 0.00675, −0.00675, 0.00675, −0.00675, 0.00674, −0.00674, −0.00674, −0.00674, 0.00674, 0.00674, −0.00674, −0.00674, −0.00674, −0.00674, 0.00674, −0.00674, 0.00674, −0.00674, 0.00673, 0.00673, −0.00673, −0.00673, 0.00672, −0.00672, 0.00672, −0.00672, 0.00672, 0.00672, 0.00672, 0.00672, 0.00672, 0.00672, 0.00672, −0.00671, −0.00671, −0.00671, −0.00671, −0.00671, 0.00671, −0.0067, −0.0067, 0.0067, 0.0067, −0.0067, 0.0067, −0.0067, 0.0067, 0.00669, 0.00669, −0.00669, −0.00669, −0.00669, 0.00669, −0.00669, −0.00669, 0.00669, 0.00668, −0.00668, 0.00668, −0.00668, 0.00668, −0.00668, −0.00668, 0.00668, 0.00667, −0.00667, 0.00667, 0.00667, 0.00667, −0.00667, −0.00667, −0.00667, 0.00666, −0.00666, 0.00666, −0.00666, 0.00666, 0.00666, −0.00666, 0.00666, −0.00666, −0.00666, −0.00666, −0.00666, 0.00666, 0.00666, −0.00666, 0.00665, 0.00665, 0.00665, 0.00665, 0.00665, 0.00664, 0.00664, 0.00664, 0.00664, 0.00664, −0.00664, −0.00664, 0.00664, −0.00664, 0.00664, 0.00664, −0.00664, −0.00664, 0.00663, −0.00663, −0.00663, −0.00663, 0.00663, −0.00663, −0.00663, 0.00663, 0.00663, −0.00662, −0.00662, −0.00662, 0.00662, −0.00662, −0.00662, 0.00662, −0.00662, 0.00662, −0.00661, −0.00661, 0.00661, 0.00661, −0.00661, 0.00661, 0.00661, 0.00661, 0.00661, −0.00661, 0.00661, −0.00661, 0.0066, −0.0066, 0.0066, 0.0066, −0.0066, −0.00659, 0.00659, −0.00659, 0.00659, 0.00659, −0.00659, −0.00659, 0.00659, 0.00659, 0.00659, 0.00659, 0.00659, 0.00659, 0.00659, 0.00659, −0.00658, −0.00658, −0.00658, 0.00658, 0.00658, 0.00658, −0.00658, 0.00658, −0.00658, 0.00658, 0.00657, 0.00657, 0.00657, 0.00657, −0.00657, −0.00657, 0.00657, −0.00657, −0.00657, 0.00657, −0.00657, 0.00657, −0.00656, 0.00656, 0.00656, 0.00656, −0.00656, 0.00656, −0.00655, 0.00655, −0.00655, −0.00655, 0.00655, −0.00655, −0.00655, 0.00655, −0.00654, 0.00654, −0.00654, −0.00654, −0.00654, −0.00653, −0.00653, 0.00653, 0.00653, −0.00653, −0.00653, −0.00653, −0.00653, 0.00653, −0.00653, −0.00653, −0.00653, 0.00653, 0.00653, −0.00652, −0.00652, −0.00652, −0.00652, 0.00652, −0.00652, −0.00652, −0.00652, −0.00652, 0.00652, −0.00652, 0.00652, −0.00652, −0.00652, 0.00651, 0.00651, 0.00651, −0.00651, 0.00651, −0.00651, 0.00651, −0.0065, 0.0065, −0.0065, 0.0065, −0.0065, 0.0065, 0.0065, 0.0065, −0.00649, −0.00649, 0.00649, 0.00649, −0.00649, −0.00649, −0.00649, 0.00649, −0.00649, 0.00649, −0.00649, 0.00648, −0.00648, 0.00648, −0.00648, 0.00648, −0.00648, 0.00648, −0.00647, 0.00647, −0.00647, −0.00647, 0.00647, −0.00647, −0.00647, −0.00647, −0.00647, 0.00646, −0.00646, 0.00646, −0.00646, 0.00646, −0.00646, 0.00646, 0.00646, 0.00646, −0.00645, 0.00645, −0.00645, −0.00645, −0.00645, −0.00645, −0.00645, 0.00645, 0.00645, −0.00645, 0.00644, 0.00644, 0.00644, 0.00644, 0.00644, 0.00644, −0.00644, 0.00643, −0.00643, 0.00643, −0.00643, 0.00643, 0.00643, −0.00643, 0.00643, 0.00643, 0.00642, 0.00642, 0.00642, 0.00642, 0.00642, 0.00642, 0.00642, 0.00642, 0.00642, 0.00642, −0.00641, 0.00641, 0.00641, −0.00641, −0.00641, −0.00641, −0.00641, 0.00641, −0.00641, −0.00641, −0.00641, −0.00641, 0.00641, 0.0064, −0.0064, 0.0064, −0.0064, 0.0064, 0.0064, 0.0064, 0.00639, 0.00639, −0.00639, −0.00639, 0.00639, 0.00639, 0.00639, 0.00639, 0.00639, −0.00639, −0.00638, 0.00638, 0.00638, 0.00638, 0.00638, −0.00638, 0.00638, 0.00638, −0.00638, 0.00638, −0.00638, 0.00638, −0.00637, 0.00637, 0.00637, −0.00637, −0.00637, −0.00637, 0.00637, −0.00636, −0.00636, −0.00636, −0.00636, −0.00636, −0.00635, −0.00635, −0.00635, −0.00635, 0.00635, −0.00635, −0.00635, −0.00635, −0.00635, −0.00635, −0.00635, −0.00634, 0.00634, 0.00634, −0.00634, 0.00634, −0.00634, −0.00634, 0.00634, −0.00634, 0.00633, −0.00633, −0.00633, 0.00633, 0.00633, −0.00633, −0.00633, −0.00633, 0.00633, −0.00632, 0.00632, 0.00632, 0.00632, 0.00632, 0.00632, 0.00632, 0.00632, 0.00632, −0.00632, 0.00632, 0.00632, 0.00632, 0.00632, −0.00632, −0.00632, 0.00631, −0.00631, −0.00631, 0.00631, −0.0063, 0.0063, −0.0063, 0.0063, 0.0063, 0.0063, −0.00629, 0.00629, −0.00629, −0.00628, −0.00628, −0.00628, 0.00628, 0.00627, 0.00627, 0.00627, −0.00627, −0.00627, 0.00627, 0.00627, −0.00626, −0.00626, −0.00626, 0.00626, 0.00626, −0.00626, −0.00626, 0.00626, 0.00626, −0.00625, 0.00625, −0.00625, −0.00625, 0.00625, −0.00624, −0.00624, 0.00624, 0.00624, 0.00624, −0.00624, −0.00624, 0.00624, 0.00624, −0.00624, 0.00624, −0.00623, 0.00623, 0.00623, 0.00623, 0.00623, −0.00623, −0.00623, 0.00623, 0.00622, 0.00622, 0.00622, −0.00622, −0.00622, −0.00622, −0.00622, −0.00622, −0.00621, 0.00621, 0.00621, −0.00621, −0.00621, 0.00621, 0.00621, −0.00621, 0.0062, 0.0062, −0.0062, −0.0062, 0.0062, 0.0062, 0.0062, 0.0062, −0.0062, −0.0062, −0.00619, 0.00619, 0.00619, 0.00619, −0.00619, −0.00619, −0.00619, −0.00619, 0.00619, 0.00619, 0.00619, 0.00618, −0.00618, 0.00618, 0.00618, 0.00618, 0.00618, 0.00618, 0.00618, −0.00618, 0.00618, −0.00617, −0.00617, −0.00617, −0.00617, −0.00617, −0.00617, 0.00617, 0.00617, 0.00617, 0.00616, 0.00616, 0.00616, −0.00616, 0.00616, −0.00616, 0.00616, −0.00616, −0.00616, 0.00616, 0.00616, −0.00616, 0.00615, 0.00615, −0.00615, −0.00615, 0.00615, −0.00615, 0.00615, −0.00615, 0.00615, −0.00615, 0.00615, −0.00615, −0.00615, 0.00615, 0.00615, 0.00614, 0.00614, 0.00614, −0.00614, 0.00614, −0.00614, −0.00614, 0.00614, 0.00613, 0.00613, 0.00613, −0.00613, 0.00613, −0.00613, −0.00613, 0.00612, −0.00612, 0.00612, 0.00612, 0.00612, −0.00612, 0.00612, 0.00612, 0.00611, −0.00611, −0.00611, −0.00611, 0.00611, 0.00611, 0.00611, −0.00611, −0.00611, 0.0061, 0.0061, −0.0061, 0.0061, 0.0061, −0.0061, 0.0061, 0.0061, −0.0061, 0.0061, −0.0061, −0.0061, −0.00609, −0.00609, 0.00609, 0.00609, 0.00609, −0.00609, −0.00609, −0.00608, 0.00608, −0.00608, −0.00608, 0.00608, 0.00608, 0.00608, −0.00608, −0.00608, 0.00608, 0.00608, 0.00608, 0.00608, 0.00607, −0.00607, −0.00607, −0.00607, 0.00607, 0.00607, 0.00606, 0.00606, −0.00606, 0.00606, −0.00606, 0.00605, 0.00605, 0.00605, −0.00605, −0.00605, 0.00605, 0.00605, 0.00605, −0.00605, 0.00604, −0.00604, −0.00604, −0.00604, −0.00604, −0.00604, 0.00604, −0.00603, −0.00603, −0.00603, 0.00603, 0.00603, 0.00603, −0.00603, −0.00603, −0.00603, −0.00603, −0.00603, 0.00603, 0.00603, −0.00603, 0.00603, 0.00602, −0.00602, −0.00602, −0.00602, 0.00602, −0.00602, 0.00602, 0.00602, −0.00602, 0.00602, 0.00602, 0.00601, −0.00601, −0.00601, 0.00601, −0.00601, −0.00601, 0.00601, −0.006, −0.006, 0.006, −0.006, 0.006, 0.00599, −0.00599, 0.00599, −0.00599, 0.00599, −0.00599, −0.00599, 0.00599, 0.00598, 0.00598, 0.00598, 0.00598, 0.00598, −0.00598, 0.00598, 0.00598, 0.00598, −0.00598, 0.00598, −0.00598, 0.00598, −0.00597, 0.00597, −0.00597, 0.00597, 0.00597, −0.00597, 0.00597, 0.00597, −0.00597, −0.00597, 0.00597, 0.00597, −0.00597, 0.00597, 0.00597, 0.00597, −0.00597, −0.00597, −0.00597, 0.00596, −0.00596, −0.00596, −0.00596, 0.00596, −0.00596, 0.00596, 0.00596, 0.00595, −0.00595, 0.00595, −0.00595, 0.00595, 0.00595, −0.00594, 0.00594, −0.00594, −0.00594, −0.00594, 0.00593, 0.00593, −0.00593, −0.00593, −0.00593, −0.00593, −0.00593, −0.00593, 0.00593, −0.00593, 0.00593, 0.00593, 0.00592, 0.00592, 0.00592, 0.00592, −0.00592, −0.00592, −0.00592, −0.00592, −0.00592, 0.00592, 0.00592, −0.00591, 0.00591, 0.00591, 0.00591, −0.00591, −0.00591, −0.00591, −0.0059, −0.0059, −0.0059, 0.0059, −0.0059, 0.00589, 0.00589, −0.00589, 0.00589, −0.00589, −0.00589, 0.00589, 0.00589, −0.00589, 0.00589, 0.00588, −0.00588, −0.00588, 0.00588, −0.00588, −0.00588, −0.00588, 0.00587, −0.00587, 0.00587, 0.00587, −0.00587, 0.00587, 0.00587, −0.00587, −0.00587, −0.00587, 0.00586, −0.00586, 0.00586, −0.00586, 0.00586, 0.00586, 0.00586, 0.00586, −0.00586, 0.00586, 0.00586, 0.00586, 0.00586, −0.00586, 0.00586, −0.00586, −0.00586, −0.00585, −0.00585, −0.00585, 0.00585, −0.00585, −0.00585, 0.00585, −0.00585, −0.00585, 0.00585, 0.00584, 0.00584, −0.00584, 0.00584, 0.00584, 0.00584, 0.00584, 0.00583, −0.00583, 0.00583, 0.00583, 0.00583, 0.00583, −0.00583, 0.00583, −0.00583, 0.00583, 0.00583, −0.00582, 0.00582, 0.00582, 0.00582, −0.00582, −0.00582, −0.00582, −0.00581, 0.00581, 0.00581, 0.00581, −0.00581, 0.00581, 0.00581, 0.00581, −0.00581, 0.00581, −0.00581, 0.0058, −0.0058, 0.0058, 0.0058, 0.0058, 0.0058, 0.0058, 0.0058, 0.0058, 0.0058, 0.00579, −0.00579, 0.00579, −0.00579, 0.00579, −0.00579, −0.00579, 0.00579, −0.00579, −0.00579, 0.00578, 0.00578, −0.00578, −0.00578, 0.00578, −0.00578, −0.00577, −0.00577, 0.00577, −0.00577, −0.00577, 0.00577, 0.00577, 0.00577, −0.00577, 0.00576, 0.00576, −0.00576, 0.00576, 0.00576, 0.00576, 0.00576, 0.00576, 0.00575, 0.00575, 0.00575, 0.00575, −0.00575, 0.00575, −0.00575, −0.00575, −0.00575, −0.00574, −0.00574, 0.00574, −0.00574, 0.00574, 0.00574, −0.00574, −0.00574, −0.00574, 0.00574, −0.00574, 0.00574, −0.00574, −0.00574, −0.00574, −0.00573, −0.00573, 0.00573, 0.00573, 0.00573, −0.00573, 0.00573, −0.00573, 0.00573, 0.00573, −0.00573, 0.00572, 0.00572, −0.00572, 0.00572, 0.00572, 0.00572, 0.00572, −0.00572, −0.00572, −0.00572, 0.00572, −0.00572, −0.00571, −0.00571, −0.00571, 0.00571, −0.00571, 0.00571, 0.00571, 0.00571, 0.00571, −0.00571, 0.00571, 0.0057, 0.0057, −0.0057, −0.0057, −0.0057, 0.0057, 0.0057, 0.00569, −0.00569, 0.00569, 0.00569, 0.00569, −0.00569, −0.00569, 0.00568, −0.00568, 0.00568, 0.00568, −0.00568, 0.00568, −0.00568, −0.00568, −0.00568, −0.00568, 0.00567, −0.00567, −0.00567, 0.00567, 0.00567, 0.00567, −0.00567, 0.00567, 0.00567, −0.00567, −0.00567, −0.00567, −0.00566, 0.00566, −0.00566, 0.00566, −0.00566, −0.00566, −0.00566, 0.00566, −0.00566, 0.00566, 0.00566, −0.00565, −0.00565, 0.00565, −0.00565, 0.00565, 0.00565, −0.00565, 0.00565, 0.00564, 0.00564, −0.00564, −0.00564, −0.00564, 0.00564, −0.00564, −0.00564, −0.00563, −0.00563, −0.00563, −0.00563, 0.00563, −0.00563, 0.00562, 0.00562, −0.00562, 0.00562, −0.00562, 0.00562, 0.00562, 0.00562, 0.00562, −0.00562, 0.00562, 0.00561, 0.00561, 0.00561, −0.00561, 0.0056, 0.0056, 0.0056, 0.0056, 0.0056, −0.0056, −0.0056, 0.0056, 0.0056, −0.0056, 0.0056, 0.0056, 0.00559, 0.00559, 0.00559, −0.00559, −0.00559, 0.00559, −0.00559, −0.00559, −0.00559, 0.00559, −0.00559, −0.00558, 0.00558, 0.00558, −0.00558, 0.00558, 0.00558, −0.00558, −0.00558, −0.00558, −0.00558, −0.00558, −0.00557, 0.00557, −0.00557, 0.00557, −0.00557, 0.00557, 0.00556, −0.00556, −0.00556, −0.00556, 0.00556, 0.00556, −0.00556, −0.00555, −0.00555, 0.00555, −0.00555, −0.00555, 0.00555, 0.00555, −0.00555, −0.00555, 0.00555, 0.00554, −0.00554, −0.00554, 0.00554, −0.00554, −0.00554, 0.00554, 0.00554, 0.00553, −0.00553, 0.00553, 0.00553, −0.00553, −0.00553, 0.00553, 0.00553, 0.00552, 0.00552, 0.00552, 0.00552, −0.00552, 0.00552, 0.00552, 0.00552, −0.00551, −0.00551, −0.00551, 0.00551, −0.00551, −0.00551, −0.00551, −0.00551, −0.00551, −0.00551, 0.0055, 0.0055, −0.0055, 0.0055, −0.0055, 0.0055, 0.0055, 0.0055, 0.0055, 0.00549, 0.00549, −0.00549, −0.00549, 0.00549, 0.00549, −0.00549, −0.00549, 0.00549, −0.00549, 0.00549, 0.00549, 0.00549, 0.00549, −0.00549, 0.00549, −0.00549, −0.00548, −0.00548, −0.00548, 0.00548, −0.00548, 0.00548, −0.00548, −0.00548, 0.00548, −0.00547, 0.00547, −0.00547, 0.00547, −0.00547, −0.00547, 0.00546, 0.00546, −0.00546, −0.00546, −0.00546, 0.00546, 0.00546, 0.00546, 0.00546, −0.00546, −0.00546, 0.00545, −0.00545, 0.00545, 0.00545, 0.00545, 0.00545, −0.00545, 0.00545, 0.00545, −0.00545, 0.00545, −0.00544, −0.00544, 0.00544, 0.00544, 0.00544, −0.00544, 0.00544, −0.00544, 0.00543, 0.00543, −0.00543, 0.00543, −0.00543, −0.00543, −0.00543, 0.00543, −0.00542, −0.00542, 0.00542, −0.00542, −0.00542, −0.00542, −0.00542, −0.00542, −0.00542, 0.00542, −0.00542, 0.00542, −0.00541, 0.00541, 0.00541, −0.00541, 0.00541, −0.0054, 0.0054, 0.0054, 0.0054, 0.0054, 0.0054, 0.0054, 0.0054, −0.0054, −0.0054, −0.0054, 0.0054, −0.00539, 0.00539, −0.00539, −0.00539, −0.00539, 0.00539, −0.00539, 0.00539, −0.00539, −0.00539, 0.00539, −0.00538, −0.00538, −0.00538, −0.00538, 0.00538, −0.00538, 0.00538, −0.00537, −0.00537, 0.00537, 0.00537, −0.00537, −0.00537, 0.00537, 0.00537, −0.00537, −0.00536, −0.00536, −0.00536, 0.00536, −0.00536, 0.00536, −0.00536, −0.00536, 0.00536, −0.00535, 0.00535, −0.00535, 0.00535, −0.00535, −0.00535, 0.00534, 0.00534, −0.00534, 0.00534, −0.00534, 0.00534, −0.00534, −0.00534, 0.00534, −0.00534, −0.00534, 0.00533, 0.00533, 0.00533, −0.00533, 0.00533, −0.00533, −0.00532, −0.00532, −0.00532, 0.00532, −0.00532, −0.00532, 0.00532, 0.00532, −0.00532, −0.00532, 0.00532, 0.00532, 0.00531, 0.00531, −0.00531, −0.00531, −0.00531, 0.00531, −0.00531, −0.00531, 0.0053, −0.0053, −0.0053, 0.0053, 0.0053, −0.0053, −0.0053, −0.0053, −0.0053, −0.0053, −0.0053, 0.00529, −0.00529, −0.00529, −0.00529, −0.00529, −0.00529, −0.00529, −0.00529, 0.00529, 0.00529, 0.00529, 0.00529, 0.00529, 0.00529, 0.00529, 0.00529, 0.00528, −0.00528, 0.00528, 0.00528, −0.00528, −0.00528, 0.00527, −0.00527, −0.00527, −0.00527, −0.00527, −0.00527, 0.00527, 0.00527, −0.00527, −0.00527, 0.00527, 0.00527, −0.00526, 0.00526, 0.00526, 0.00526, −0.00526, 0.00526, −0.00526, −0.00526, 0.00526, −0.00526, −0.00525, −0.00525, −0.00525, 0.00525, −0.00525, 0.00525, −0.00525, −0.00525, 0.00525, −0.00525, −0.00525, −0.00525, −0.00524, 0.00524, −0.00524, 0.00524, −0.00524, 0.00524, 0.00524, −0.00523, −0.00523, 0.00523, −0.00523, −0.00523, −0.00523, 0.00523, 0.00523, −0.00523, 0.00523, −0.00522, 0.00522, −0.00522, −0.00522, −0.00522, 0.00522, −0.00522, 0.00521, −0.00521, 0.00521, 0.00521, 0.0052, 0.0052, 0.0052, 0.0052, 0.0052, −0.0052, 0.0052, 0.0052, 0.00519, 0.00519, −0.00519, −0.00519, −0.00519, −0.00519, 0.00519, 0.00519, 0.00519, 0.00518, −0.00518, −0.00518, −0.00518, 0.00518, 0.00518, −0.00518, 0.00518, −0.00518, 0.00518, 0.00518, 0.00518, 0.00518, −0.00518, 0.00518, −0.00518, −0.00518, 0.00518, −0.00517, 0.00517, 0.00517, 0.00517, −0.00517, 0.00517, −0.00517, 0.00517, 0.00517, 0.00517, −0.00516, −0.00516, −0.00516, 0.00516, −0.00516, −0.00516, −0.00516, −0.00516, 0.00515, −0.00515, 0.00515, 0.00515, 0.00515, 0.00515, 0.00515, 0.00515, −0.00515, −0.00515, −0.00515, −0.00515, −0.00515, 0.00514, 0.00514, 0.00514, −0.00514, −0.00514, 0.00514, −0.00513, 0.00513, 0.00513, 0.00513, 0.00513, 0.00513, −0.00513, 0.00513, 0.00513, −0.00513, −0.00513, 0.00513, 0.00513, 0.00513, −0.00513, −0.00513, 0.00513, 0.00512, −0.00512, 0.00512, 0.00512, 0.00512, −0.00512, −0.00512, −0.00512, −0.00512, 0.00512, −0.00512, 0.00511, 0.00511, 0.00511, −0.00511, −0.00511, 0.00511, −0.00511, 0.00511, 0.00511, 0.00511, −0.00511, 0.00511, −0.00511, −0.0051, −0.0051, 0.0051, −0.0051, 0.0051, 0.0051, −0.0051, 0.0051, −0.0051, 0.0051, 0.0051, −0.0051, 0.0051, −0.00509, −0.00509, −0.00509, 0.00509, −0.00509, −0.00509, −0.00509, 0.00509, 0.00509, −0.00509, 0.00509, 0.00508, 0.00508, 0.00508, 0.00508, −0.00508, 0.00508, 0.00507, −0.00507, −0.00507, 0.00507, −0.00507, 0.00506, 0.00506, −0.00506, −0.00506, 0.00506, −0.00506, −0.00506, 0.00506, 0.00506, 0.00505, 0.00505, 0.00505, 0.00505, −0.00505, −0.00505, 0.00505, −0.00505, −0.00505, −0.00505, 0.00505, 0.00505, 0.00505, 0.00505, −0.00504, 0.00504, 0.00504, 0.00503, −0.00503, −0.00503, −0.00503, 0.00503, 0.00503, 0.00503, 0.00503, 0.00503, 0.00502, −0.00502, 0.00502, 0.00502, −0.00502, −0.00502, −0.00501, −0.00501, 0.00501, 0.00501, 0.00501, 0.00501, −0.00501, −0.00501, −0.00501, −0.005, 0.005, 0.005, −0.005, −0.005, 0.005, 0.00499, −0.00499, 0.00499, −0.00499, −0.00499, −0.00499, −0.00499, −0.00499, 0.00499, 0.00498, −0.00498, −0.00498, −0.00498, −0.00498, −0.00498, 0.00498, −0.00498, −0.00498, −0.00498, 0.00498, 0.00497, 0.00497, −0.00497, 0.00497, −0.00497, −0.00497, 0.00497, 0.00497, 0.00497, 0.00496, −0.00496, −0.00496, 0.00496, 0.00496, 0.00496, −0.00496, 0.00496, −0.00496, −0.00496, −0.00496, −0.00496, −0.00496, 0.00496, −0.00496, −0.00495, 0.00495, −0.00495, 0.00495, −0.00495, −0.00495, −0.00495, −0.00495, −0.00494, −0.00494, 0.00494, 0.00494, 0.00494, −0.00494, −0.00494, 0.00494, 0.00494, −0.00494, 0.00494, 0.00494, 0.00493, −0.00493, −0.00493, −0.00493, −0.00493, −0.00493, 0.00493, −0.00493, −0.00493, 0.00493, 0.00492, 0.00492, 0.00492, −0.00492, −0.00491, −0.00491, 0.00491, 0.00491, 0.0049, 0.0049, 0.0049, −0.0049, −0.0049, −0.0049, 0.0049, 0.0049, −0.0049, −0.0049, 0.00489, 0.00489, −0.00489, 0.00489, −0.00489, −0.00489, 0.00489, 0.00489, −0.00489, 0.00488, −0.00488, 0.00488, 0.00488, 0.00488, −0.00488, −0.00488, −0.00488, −0.00488, −0.00488, 0.00488, −0.00488, −0.00488, 0.00488, 0.00487, 0.00487, 0.00487, −0.00487, −0.00487, −0.00487, −0.00487, 0.00487, −0.00487, 0.00486, −0.00486, −0.00486, 0.00486, 0.00486, 0.00486, 0.00486, 0.00486, −0.00486, 0.00486, −0.00486, 0.00486, 0.00486, 0.00486, −0.00486, −0.00485, 0.00485, −0.00485, −0.00485, −0.00485, −0.00485, 0.00484, −0.00484, −0.00484, 0.00484, 0.00484, −0.00483, −0.00483, −0.00483, 0.00483, 0.00483, −0.00483, 0.00483, 0.00483, 0.00483, 0.00483, −0.00482, −0.00482, −0.00482, −0.00482, −0.00482, −0.00482, −0.00482, −0.00482, −0.00482, 0.00482, 0.00482, 0.00482, −0.00482, −0.00482, −0.00481, 0.00481, 0.00481, −0.00481, 0.00481, −0.00481, 0.00481, 0.00481, −0.0048, −0.0048, 0.0048, 0.0048, −0.0048, −0.0048, −0.0048, 0.0048, 0.0048, −0.0048, −0.0048, −0.00479, −0.00479, 0.00479, −0.00479, −0.00479, −0.00479, 0.00479, 0.00479, 0.00478, −0.00478, −0.00478, 0.00478, 0.00478, −0.00478, −0.00478, 0.00478, 0.00478, 0.00478, −0.00478, 0.00477, 0.00477, 0.00477, −0.00477, −0.00477, −0.00477, 0.00477, −0.00477, −0.00477, 0.00477, 0.00477, 0.00476, −0.00476, 0.00476, 0.00476, 0.00476, 0.00476, 0.00476, 0.00476, −0.00476, −0.00476, −0.00476, 0.00476, 0.00475, 0.00475, 0.00475, 0.00475, −0.00475, 0.00475, −0.00475, −0.00475, −0.00475, −0.00475, −0.00475, −0.00474, −0.00474, −0.00474, 0.00474, 0.00474, −0.00474, −0.00474, 0.00474, −0.00474, −0.00474, 0.00474, 0.00474, 0.00474, −0.00474, −0.00473, −0.00473, −0.00473, 0.00473, −0.00473, −0.00473, −0.00473, −0.00473, 0.00473, 0.00472, 0.00472, 0.00472, 0.00472, −0.00472, 0.00471, 0.00471, 0.00471, 0.00471, 0.00471, −0.00471, 0.00471, −0.00471, −0.0047, −0.0047, −0.0047, −0.0047, −0.0047, 0.0047, 0.0047, 0.0047, −0.0047, 0.0047, −0.00469, −0.00469, 0.00469, 0.00469, 0.00469, −0.00469, −0.00468, 0.00468, 0.00468, −0.00468, 0.00468, −0.00468, 0.00468, −0.00468, 0.00467, 0.00467, −0.00467, −0.00467, 0.00467, 0.00467, −0.00466, −0.00466, 0.00466, 0.00466, 0.00466, 0.00466, −0.00465, −0.00465, −0.00465, 0.00465, 0.00465, 0.00465, 0.00465, 0.00465, 0.00465, −0.00465, 0.00465, 0.00465, 0.00464, −0.00464, −0.00464, −0.00464, 0.00464, −0.00464, −0.00464, −0.00464, −0.00464, −0.00464, 0.00464, 0.00464, 0.00464, −0.00464, 0.00463, 0.00463, −0.00463, −0.00463, 0.00463, 0.00463, −0.00463, 0.00463, 0.00462, −0.00462, 0.00462, −0.00462, 0.00462, 0.00462, 0.00462, 0.00462, −0.00462, −0.00462, 0.00462, 0.00461, 0.00461, −0.00461, 0.00461, 0.00461, −0.0046, −0.0046, −0.0046, 0.0046, 0.0046, −0.0046, 0.0046, 0.0046, 0.0046, 0.0046, −0.00459, 0.00459, −0.00459, 0.00459, −0.00459, −0.00459, −0.00459, −0.00459, −0.00459, −0.00459, 0.00458, 0.00458, −0.00458, −0.00458, 0.00458, 0.00458, 0.00457, 0.00457, −0.00457, −0.00457, −0.00457, −0.00457, −0.00457, 0.00457, −0.00456, 0.00456, 0.00456, −0.00456, −0.00456, 0.00456, −0.00455, 0.00455, −0.00455, 0.00455, −0.00455, 0.00455, −0.00455, 0.00454, 0.00454, −0.00454, −0.00454, 0.00454, 0.00453, 0.00453, 0.00453, −0.00453, 0.00453, −0.00453, 0.00453, −0.00453, −0.00453, 0.00453, −0.00453, 0.00453, −0.00452, −0.00452, 0.00452, −0.00452, 0.00452, −0.00452, 0.00451, −0.00451, 0.00451, −0.00451, −0.00451, −0.00451, 0.00451, −0.00451, 0.00451, −0.0045, 0.0045, −0.0045, −0.0045, 0.0045, −0.0045, 0.00449, 0.00449, 0.00449, 0.00449, −0.00449, 0.00449, 0.00449, −0.00449, −0.00449, 0.00449, 0.00449, −0.00449, −0.00449, 0.00449, −0.00449, 0.00449, −0.00449, 0.00448, 0.00448, −0.00448, 0.00448, −0.00448, −0.00448, 0.00448, 0.00448, −0.00448, −0.00448, −0.00448, 0.00447, −0.00447, −0.00447, 0.00447, 0.00447, −0.00447, 0.00447, 0.00447, −0.00447, 0.00447, 0.00447, 0.00446, −0.00446, 0.00446, 0.00446, −0.00446, 0.00446, −0.00446, −0.00446, 0.00446, 0.00445, −0.00445, −0.00445, −0.00445, 0.00445, −0.00445, −0.00445, −0.00445, 0.00445, 0.00445, 0.00445, −0.00445, 0.00445, 0.00445, −0.00445, −0.00445, 0.00444, −0.00444, −0.00444, −0.00444, −0.00444, −0.00444, 0.00444, 0.00444, −0.00444, 0.00444, −0.00443, 0.00443, 0.00443, 0.00443, −0.00443, 0.00443, 0.00443, −0.00443, 0.00443, 0.00443, −0.00443, −0.00442, 0.00442, −0.00442, 0.00442, 0.00442, −0.00442, 0.00442, −0.00442, 0.00442, 0.00442, −0.00442, −0.00442, −0.00442, −0.00442, 0.00441, 0.00441, 0.00441, 0.00441, 0.00441, 0.00441, −0.00441, −0.00441, −0.00441, 0.00441, −0.00441, −0.00441, 0.0044, 0.0044, −0.0044, −0.0044, 0.0044, 0.0044, −0.0044, 0.0044, 0.00439, 0.00439, −0.00439, 0.00439, −0.00439, −0.00439, 0.00438, 0.00438, −0.00438, 0.00438, 0.00438, 0.00438, 0.00438, −0.00438, 0.00438, −0.00438, −0.00438, 0.00438, 0.00438, 0.00438, −0.00437, 0.00437, 0.00437, 0.00437, −0.00437, 0.00437, −0.00437, 0.00437, 0.00437, 0.00437, −0.00437, 0.00437, −0.00436, 0.00436, −0.00436, −0.00436, 0.00436, 0.00436, 0.00436, 0.00436, 0.00436, 0.00436, −0.00436, −0.00436, −0.00436, 0.00436, −0.00436, 0.00436, −0.00435, −0.00435, 0.00435, 0.00435, 0.00435, −0.00435, 0.00435, 0.00435, −0.00434, −0.00434, 0.00434, −0.00434, −0.00434, 0.00434, 0.00434, −0.00434, −0.00434, 0.00433, −0.00433, 0.00433, −0.00433, −0.00433, 0.00433, −0.00433, 0.00433, 0.00432, −0.00432, 0.00432, −0.00432, 0.00432, 0.00432, 0.00432, −0.00432, 0.00432, 0.00432, 0.00432, 0.00432, 0.00432, 0.00431, 0.00431, 0.00431, 0.00431, 0.00431, −0.00431, 0.00431, −0.00431, −0.00431, −0.00431, −0.00431, −0.00431, 0.00431, 0.00431, 0.0043, 0.0043, −0.0043, 0.0043, 0.00429, 0.00429, −0.00429, −0.00429, −0.00429, −0.00428, −0.00428, −0.00428, −0.00428, 0.00428, −0.00428, 0.00428, −0.00428, 0.00428, −0.00428, −0.00427, 0.00427, 0.00427, −0.00427, −0.00427, −0.00427, −0.00427, 0.00427, −0.00427, −0.00427, −0.00426, 0.00426, −0.00426, −0.00426, 0.00426, −0.00426, −0.00426, 0.00425, −0.00425, −0.00425, −0.00425, 0.00425, 0.00425, 0.00425, −0.00425, −0.00425, −0.00425, 0.00424, −0.00424, −0.00424, 0.00424, 0.00424, 0.00424, −0.00424, 0.00424, −0.00424, 0.00423, −0.00423, −0.00423, 0.00423, −0.00423, 0.00423, −0.00423, 0.00423, −0.00423, −0.00423, 0.00423, −0.00423, 0.00423, 0.00423, 0.00423, 0.00423, −0.00423, −0.00422, −0.00422, −0.00422, −0.00422, −0.00422, 0.00422, 0.00422, −0.00422, −0.00421, −0.00421, 0.00421, −0.00421, −0.00421, −0.00421, −0.00421, 0.00421, 0.00421, −0.0042, 0.0042, 0.0042, −0.0042, −0.0042, 0.0042, 0.00419, 0.00419, −0.00419, 0.00419, 0.00419, 0.00419, 0.00418, 0.00418, 0.00418, −0.00418, 0.00418, −0.00418, −0.00418, −0.00418, 0.00418, 0.00418, −0.00418, 0.00418, 0.00418, −0.00417, 0.00417, −0.00417, 0.00417, −0.00417, −0.00417, 0.00417, 0.00417, 0.00417, −0.00417, −0.00417, −0.00417, −0.00416, −0.00416, 0.00416, −0.00416, 0.00416, 0.00416, 0.00416, −0.00416, 0.00416, −0.00416, −0.00416, 0.00415, 0.00415, 0.00415, 0.00415, 0.00415, −0.00415, 0.00415, −0.00414, 0.00414, −0.00414, 0.00414, 0.00414, −0.00414, 0.00414, 0.00414, 0.00413, −0.00413, 0.00413, 0.00413, 0.00413, −0.00413, −0.00413, −0.00412, 0.00412, −0.00412, 0.00412, 0.00412, 0.00412, 0.00412, −0.00412, 0.00412, −0.00412, 0.00412, −0.00412, −0.00411, 0.00411, −0.00411, −0.00411, −0.00411, 0.00411, −0.00411, −0.00411, −0.00411, 0.0041, −0.0041, 0.0041, 0.0041, −0.0041, −0.0041, −0.0041, −0.0041, −0.0041, −0.0041, −0.0041, −0.0041, 0.0041, −0.00409, 0.00409, 0.00409, 0.00409, −0.00409, −0.00409, 0.00409, −0.00409, 0.00409, −0.00409, −0.00409, 0.00409, −0.00409, 0.00408, −0.00408, 0.00408, −0.00408, −0.00408, 0.00408, −0.00408, 0.00408, 0.00407, 0.00407, 0.00407, −0.00407, 0.00407, −0.00406, 0.00406, −0.00406, 0.00406, 0.00406, −0.00406, 0.00405, −0.00405, 0.00405, 0.00405, 0.00405, −0.00405, 0.00405, 0.00404, 0.00404, −0.00404, −0.00404, −0.00404, −0.00404, 0.00404, −0.00404, 0.00404, 0.00404, −0.00404, −0.00404, 0.00404, −0.00404, −0.00404, 0.00403, 0.00403, −0.00403, 0.00403, 0.00403, −0.00403, 0.00403, 0.00403, 0.00403, 0.00403, 0.00403, 0.00403, 0.00403, 0.00403, 0.00402, 0.00402, −0.00402, −0.00402, 0.00402, 0.00402, −0.00402, −0.00402, 0.00402, −0.00402, −0.00402, 0.00401, −0.00401, −0.00401, −0.00401, 0.00401, 0.00401, −0.00401, 0.004, −0.004, 0.004, −0.004, 0.004, −0.004, −0.004, 0.004, −0.004, −0.004, −0.004, 0.004, −0.00399, 0.00399, 0.00399, 0.00399, −0.00399, 0.00399, −0.00399, −0.00399, 0.00399, −0.00399, −0.00399, −0.00399, −0.00398, −0.00398, −0.00398, 0.00398, −0.00398, 0.00398, −0.00398, −0.00398, 0.00398, −0.00398, −0.00397, 0.00397, −0.00397, 0.00397, 0.00397, 0.00397, 0.00397, −0.00397, 0.00397, 0.00397, −0.00397, 0.00397, 0.00397, −0.00396, 0.00396, 0.00396, −0.00396, −0.00396, 0.00396, 0.00396, −0.00395, 0.00395, 0.00395, −0.00395, 0.00395, 0.00395, 0.00395, 0.00395, −0.00394, 0.00394, −0.00394, −0.00394, 0.00394, −0.00394, −0.00394, −0.00394, −0.00394, 0.00394, −0.00394, −0.00394, −0.00394, 0.00393, 0.00393, 0.00393, 0.00393, 0.00393, −0.00393, −0.00393, −0.00392, 0.00392, 0.00392, 0.00392, 0.00392, −0.00392, 0.00392, −0.00392, 0.00392, 0.00391, −0.00391, 0.00391, −0.00391, 0.00391, −0.00391, 0.00391, −0.00391, 0.0039, 0.0039, −0.0039, 0.0039, 0.0039, −0.0039, −0.0039, −0.0039, 0.0039, −0.0039, 0.0039, 0.00389, 0.00389, 0.00389, 0.00389, −0.00389, 0.00389, −0.00389, −0.00389, −0.00389, 0.00389, −0.00389, −0.00389, 0.00388, 0.00388, 0.00388, 0.00388, −0.00388, 0.00388, −0.00387, −0.00387, 0.00387, −0.00387, −0.00387, 0.00387, 0.00387, 0.00387, 0.00387, −0.00387, −0.00387, 0.00387, 0.00387, −0.00387, −0.00387, 0.00386, −0.00386, −0.00386, −0.00386, 0.00386, 0.00386, 0.00386, −0.00385, −0.00385, 0.00385, 0.00385, −0.00385, 0.00385, −0.00385, 0.00385, 0.00385, −0.00385, 0.00385, −0.00385, 0.00384, 0.00384, 0.00384, −0.00384, −0.00384, 0.00384, 0.00384, 0.00384, 0.00384, 0.00384, 0.00384, −0.00384, −0.00384, −0.00384, 0.00384, −0.00383, −0.00383, −0.00383, 0.00383, 0.00383, −0.00383, −0.00382, −0.00382, 0.00382, −0.00382, 0.00382, −0.00382, 0.00382, −0.00382, 0.00382, 0.00382, 0.00382, 0.00381, −0.00381, 0.00381, −0.00381, −0.00381, 0.00381, −0.00381, −0.0038, −0.0038, 0.0038, −0.0038, −0.0038, −0.0038, −0.0038, −0.0038, 0.0038, 0.0038, 0.0038, −0.00379, 0.00379, −0.00379, −0.00379, −0.00379, −0.00379, −0.00379, −0.00379, 0.00379, 0.00379, −0.00379, −0.00379, −0.00378, 0.00378, −0.00378, 0.00378, 0.00378, 0.00378, −0.00378, 0.00378, 0.00378, 0.00378, −0.00378, −0.00378, 0.00377, 0.00377, −0.00377, 0.00377, 0.00377, 0.00377, 0.00377, −0.00377, 0.00376, 0.00376, −0.00376, −0.00376, 0.00376, −0.00376, 0.00376, −0.00376, 0.00375, 0.00375, 0.00375, −0.00375, 0.00375, −0.00375, −0.00375, 0.00375, 0.00375, 0.00375, 0.00375, −0.00374, 0.00374, −0.00374, 0.00374, −0.00374, −0.00374, 0.00374, −0.00374, 0.00374, 0.00374, 0.00374, 0.00374, −0.00374, 0.00374, −0.00374, 0.00374, 0.00373, 0.00373, −0.00373, −0.00373, 0.00373, 0.00373, 0.00373, 0.00373, 0.00373, −0.00373, 0.00372, −0.00372, 0.00372, 0.00372, −0.00372, 0.00372, −0.00372, −0.00372, 0.00372, −0.00371, −0.00371, 0.00371, −0.00371, −0.00371, −0.0037, 0.0037, 0.0037, 0.0037, 0.0037, 0.0037, 0.0037, 0.0037, 0.00369, 0.00369, −0.00369, −0.00369, 0.00369, 0.00369, 0.00369, −0.00369, 0.00369, 0.00368, 0.00368, 0.00368, 0.00368, 0.00368, 0.00368, 0.00368, 0.00368, 0.00368, −0.00368, −0.00368, 0.00367, −0.00367, 0.00367, −0.00367, 0.00367, 0.00367, 0.00367, −0.00367, −0.00366, 0.00366, −0.00366, −0.00366, 0.00366, −0.00365, −0.00365, −0.00365, −0.00365, −0.00365, 0.00365, 0.00365, 0.00365, −0.00365, −0.00365, −0.00365, −0.00364, 0.00364, −0.00364, 0.00364, −0.00364, 0.00364, 0.00364, 0.00364, −0.00364, 0.00363, 0.00363, −0.00363, −0.00363, −0.00363, −0.00363, 0.00363, −0.00363, −0.00363, 0.00362, 0.00362, 0.00362, −0.00362, 0.00362, 0.00362, 0.00362, −0.00362, 0.00362, −0.00362, −0.00362, 0.00361, 0.00361, 0.00361, −0.00361, −0.00361, −0.00361, 0.00361, −0.00361, 0.00361, −0.00361, 0.00361, −0.0036, 0.0036, 0.0036, −0.0036, −0.00359, 0.00359, 0.00359, 0.00359, 0.00359, −0.00359, 0.00359, 0.00359, 0.00359, 0.00359, −0.00359, −0.00359, 0.00358, −0.00358, −0.00358, 0.00358, 0.00358, 0.00358, 0.00358, 0.00358, 0.00358, −0.00358, −0.00358, −0.00358, 0.00358, 0.00358, 0.00358, −0.00358, −0.00358, 0.00357, −0.00357, −0.00357, 0.00357, 0.00357, −0.00357, 0.00357, −0.00357, 0.00357, 0.00356, 0.00356, 0.00356, −0.00356, 0.00356, 0.00356, 0.00356, 0.00356, −0.00356, 0.00356, −0.00356, −0.00355, 0.00355, −0.00355, 0.00355, −0.00355, −0.00355, −0.00355, 0.00355, 0.00355, 0.00355, −0.00354, −0.00354, −0.00354, 0.00354, −0.00354, −0.00354, −0.00354, 0.00354, 0.00354, 0.00354, 0.00354, 0.00353, −0.00353, −0.00353, −0.00353, −0.00353, −0.00353, 0.00353, 0.00353, −0.00353, −0.00353, −0.00352, 0.00352, −0.00352, 0.00352, 0.00352, 0.00352, −0.00352, 0.00352, −0.00352, −0.00352, 0.00352, 0.00351, 0.00351, −0.00351, 0.00351, 0.00351, −0.00351, 0.00351, 0.00351, 0.00351, 0.00351, −0.00351, 0.00351, 0.00351, −0.00351, −0.0035, 0.0035, −0.0035, −0.0035, 0.0035, 0.0035, 0.0035, −0.0035, −0.0035, −0.0035, −0.0035, −0.00349, 0.00349, −0.00349, 0.00349, −0.00349, 0.00349, −0.00349, −0.00349, −0.00349, 0.00349, −0.00348, −0.00348, 0.00348, −0.00348, −0.00348, 0.00348, 0.00348, 0.00348, 0.00347, 0.00347, −0.00347, −0.00347, 0.00347, −0.00347, −0.00347, 0.00347, −0.00346, −0.00346, 0.00346, −0.00346, 0.00346, 0.00346, 0.00346, 0.00346, −0.00346, −0.00346, −0.00346, 0.00346, 0.00346, −0.00346, 0.00346, −0.00345, 0.00345, 0.00345, 0.00345, −0.00345, 0.00345, −0.00345, −0.00345, 0.00344, 0.00344, 0.00344, 0.00344, 0.00344, −0.00344, −0.00344, −0.00344, −0.00344, 0.00344, −0.00344, −0.00344, 0.00343, −0.00343, 0.00343, −0.00343, −0.00343, −0.00343, 0.00343, −0.00343, 0.00343, −0.00343, 0.00343, −0.00343, 0.00342, −0.00342, −0.00342, −0.00342, −0.00342, 0.00342, 0.00342, −0.00342, −0.00342, −0.00342, 0.00342, −0.00342, −0.00341, −0.00341, 0.00341, 0.00341, −0.00341, 0.00341, 0.00341, 0.00341, −0.00341, 0.00341, −0.00341, 0.00341, 0.00341, −0.0034, 0.0034, 0.0034, 0.0034, 0.0034, −0.0034, 0.0034, 0.0034, −0.0034, 0.0034, 0.0034, −0.00339, 0.00339, 0.00339, 0.00339, 0.00339, −0.00338, 0.00338, −0.00338, −0.00338, −0.00338, 0.00338, 0.00338, −0.00338, −0.00338, −0.00338, 0.00338, 0.00338, −0.00338, 0.00338, 0.00338, 0.00338, −0.00337, −0.00337, −0.00337, −0.00337, 0.00337, 0.00337, 0.00337, −0.00337, 0.00336, 0.00336, 0.00336, 0.00336, 0.00336, −0.00336, −0.00336, −0.00336, −0.00336, 0.00336, 0.00336, −0.00336, 0.00336, 0.00336, 0.00335, 0.00335, 0.00335, 0.00335, 0.00335, −0.00335, −0.00335, −0.00335, 0.00335, −0.00334, 0.00334, 0.00334, −0.00334, 0.00334, −0.00334, 0.00334, 0.00334, 0.00334, 0.00334, 0.00334, 0.00334, 0.00334, 0.00334, −0.00334, −0.00334, 0.00334, 0.00334, 0.00333, 0.00333, 0.00333, 0.00333, 0.00333, 0.00333, −0.00333, 0.00333, 0.00333, −0.00333, 0.00333, −0.00332, −0.00332, 0.00332, 0.00332, −0.00332, −0.00332, 0.00332, 0.00331, −0.00331, −0.00331, 0.00331, 0.00331, 0.00331, 0.00331, 0.00331, 0.00331, 0.0033, 0.0033, 0.0033, 0.0033, 0.0033, −0.0033, 0.0033, 0.0033, −0.0033, 0.0033, 0.00329, −0.00329, −0.00329, −0.00329, 0.00329, −0.00329, 0.00329, 0.00329, −0.00328, −0.00328, 0.00328, −0.00328, −0.00328, −0.00328, −0.00328, 0.00328, −0.00328, −0.00328, −0.00328, 0.00328, 0.00327, 0.00327, 0.00327, 0.00327, 0.00327, −0.00327, 0.00327, 0.00327, −0.00327, 0.00327, −0.00327, 0.00327, 0.00327, −0.00327, −0.00327, 0.00327, −0.00326, 0.00326, 0.00326, 0.00326, 0.00326, −0.00326, −0.00326, −0.00326, −0.00326, −0.00325, −0.00325, 0.00325, 0.00325, 0.00325, 0.00324, −0.00324, 0.00324, 0.00324, 0.00324, −0.00324, 0.00324, 0.00324, 0.00324, 0.00323, −0.00323, −0.00323, −0.00323, 0.00323, −0.00323, −0.00323, −0.00323, −0.00323, 0.00323, 0.00323, 0.00322, −0.00322, −0.00322, −0.00322, −0.00322, −0.00322, −0.00322, −0.00322, 0.00322, −0.00321, 0.00321, 0.00321, −0.00321, −0.00321, 0.00321, −0.00321, −0.00321, −0.00321, −0.0032, −0.0032, 0.0032, −0.0032, 0.0032, −0.0032, 0.0032, 0.0032, 0.0032, 0.00319, 0.00319, −0.00319, −0.00319, 0.00319, 0.00319, −0.00319, 0.00319, 0.00319, −0.00319, 0.00319, 0.00318, −0.00318, 0.00318, −0.00318, 0.00318, −0.00318, 0.00318, 0.00318, 0.00318, 0.00318, 0.00318, 0.00318, 0.00317, −0.00317, 0.00317, 0.00317, −0.00317, −0.00317, 0.00316, 0.00316, 0.00316, 0.00316, −0.00316, −0.00316, 0.00316, 0.00316, 0.00316, −0.00316, −0.00316, 0.00315, −0.00315, −0.00315, −0.00315, −0.00315, −0.00315, 0.00314, −0.00314, 0.00314, 0.00314, −0.00314, 0.00314, 0.00314, 0.00314, 0.00314, −0.00314, 0.00314, 0.00313, −0.00313, 0.00313, −0.00313, −0.00313, −0.00313, 0.00313, −0.00313, 0.00313, 0.00313, −0.00313, −0.00312, −0.00312, 0.00312, 0.00312, −0.00312, 0.00312, 0.00312, −0.00312, 0.00312, −0.00312, −0.00312, 0.00312, 0.00312, −0.00311, −0.00311, 0.00311, −0.00311, 0.00311, 0.00311, 0.00311, −0.00311, 0.0031, 0.0031, 0.0031, 0.0031, −0.0031, 0.00309, −0.00309, 0.00309, −0.00309, −0.00309, −0.00309, 0.00309, 0.00309, −0.00308, 0.00308, −0.00308, −0.00308, −0.00308, 0.00308, 0.00308, 0.00308, −0.00307, 0.00307, −0.00307, 0.00307, 0.00307, 0.00307, −0.00307, 0.00307, 0.00307, −0.00307, 0.00306, −0.00306, −0.00306, 0.00306, 0.00306, −0.00306, 0.00306, −0.00306, −0.00306, −0.00306, 0.00306, −0.00305, −0.00305, −0.00305, −0.00305, 0.00305, −0.00305, 0.00305, 0.00305, −0.00304, −0.00304, 0.00304, 0.00304, 0.00304, 0.00304, 0.00304, 0.00304, 0.00304, −0.00304, −0.00303, 0.00303, 0.00303, 0.00303, 0.00303, −0.00303, 0.00303, −0.00303, 0.00303, −0.00302, 0.00302, 0.00302, 0.00302, −0.00302, 0.00302, −0.00302, 0.00302, 0.00302, 0.00302, 0.00301, 0.00301, 0.00301, −0.00301, 0.00301, −0.00301, −0.00301, −0.003, 0.003, 0.003, −0.003, −0.003, −0.003, 0.003, −0.003, −0.003, 0.003, −0.003, 0.00299, −0.00299, 0.00299, 0.00299, 0.00299, −0.00299, 0.00299, −0.00299, −0.00299, −0.00298, 0.00298, 0.00298, −0.00298, −0.00298, −0.00298, 0.00298, 0.00298, 0.00297, 0.00297, −0.00297, 0.00297, 0.00297, −0.00297, −0.00296, −0.00296, 0.00296, 0.00296, −0.00296, 0.00296, −0.00296, 0.00296, −0.00296, −0.00296, 0.00296, −0.00296, −0.00295, 0.00295, −0.00295, 0.00294, 0.00294, −0.00294, −0.00294, 0.00294, −0.00294, 0.00293, −0.00293, −0.00293, 0.00293, 0.00293, 0.00292, 0.00292, 0.00292, −0.00292, −0.00292, 0.00292, 0.00292, 0.00292, −0.00292, −0.00292, −0.00291, −0.00291, −0.00291, −0.00291, 0.00291, 0.00291, 0.00291, 0.00291, −0.0029, 0.0029, −0.0029, 0.0029, −0.0029, 0.0029, 0.0029, 0.0029, 0.0029, 0.0029, −0.00289, −0.00289, 0.00289, −0.00289, 0.00289, 0.00289, −0.00289, −0.00289, −0.00289, 0.00289, −0.00289, −0.00288, 0.00288, −0.00288, 0.00288, −0.00287, −0.00287, −0.00287, 0.00287, 0.00287, −0.00287, −0.00287, −0.00287, 0.00287, 0.00287, −0.00287, −0.00287, 0.00287, 0.00286, −0.00286, −0.00286, −0.00286, −0.00286, −0.00286, 0.00286, 0.00286, 0.00286, 0.00286, −0.00286, 0.00286, 0.00286, 0.00286, 0.00285, 0.00285, −0.00285, −0.00285, −0.00284, −0.00284, −0.00284, 0.00284, −0.00284, 0.00284, 0.00284, −0.00284, 0.00284, 0.00284, 0.00283, −0.00283, 0.00283, 0.00283, −0.00283, −0.00283, 0.00283, −0.00283, −0.00283, 0.00283, −0.00283, 0.00283, 0.00282, 0.00282, 0.00282, −0.00282, −0.00282, 0.00282, 0.00282, −0.00282, 0.00282, 0.00282, −0.00282, −0.00282, 0.00281, −0.00281, −0.00281, 0.00281, −0.00281, −0.00281, −0.00281, 0.00281, −0.00281, 0.00281, 0.00281, 0.00281, 0.00281, −0.0028, 0.0028, 0.0028, 0.0028, −0.0028, −0.0028, −0.00279, 0.00279, 0.00279, −0.00279, 0.00279, 0.00279, −0.00279, 0.00279, 0.00279, 0.00279, 0.00279, 0.00279, −0.00278, 0.00278, −0.00278, 0.00278, −0.00278, 0.00278, −0.00278, 0.00278, 0.00277, −0.00277, −0.00277, 0.00277, −0.00277, −0.00277, −0.00277, 0.00277, 0.00277, 0.00277, −0.00277, 0.00277, 0.00277, 0.00277, 0.00277, −0.00277, 0.00277, −0.00277, −0.00276, 0.00276, −0.00276, −0.00276, −0.00276, −0.00276, −0.00276, 0.00276, −0.00276, 0.00276, 0.00276, −0.00275, −0.00275, −0.00275, −0.00275, 0.00275, −0.00275, −0.00275, −0.00275, 0.00275, 0.00275, 0.00275, 0.00275, −0.00275, 0.00275, 0.00274, −0.00274, −0.00274, 0.00274, 0.00274, 0.00274, 0.00273, 0.00273, −0.00273, 0.00273, −0.00273, 0.00273, 0.00273, −0.00273, −0.00273, 0.00273, 0.00272, 0.00272, 0.00272, 0.00272, −0.00272, 0.00272, 0.00272, 0.00272, 0.00272, −0.00271, 0.00271, 0.00271, −0.00271, 0.0027, 0.0027, −0.0027, 0.0027, −0.0027, 0.0027, −0.0027, 0.0027, 0.0027, −0.0027, 0.00269, −0.00269, 0.00269, 0.00269, −0.00269, 0.00269, 0.00268, −0.00268, −0.00268, −0.00268, 0.00268, −0.00267, −0.00267, 0.00267, −0.00267, −0.00267, 0.00267, 0.00267, −0.00267, 0.00267, 0.00267, −0.00267, 0.00266, 0.00266, 0.00266, −0.00266, 0.00266, 0.00266, −0.00265, −0.00265, 0.00265, 0.00265, −0.00265, −0.00265, 0.00265, 0.00265, 0.00264, 0.00264, −0.00264, −0.00264, −0.00264, −0.00264, −0.00264, 0.00264, 0.00264, −0.00264, 0.00264, 0.00264, −0.00263, −0.00263, 0.00263, −0.00263, −0.00263, −0.00263, 0.00263, 0.00263, −0.00263, −0.00263, −0.00263, −0.00262, −0.00262, 0.00262, 0.00262, 0.00262, −0.00262, 0.00262, −0.00262, 0.00262, 0.00262, −0.00262, −0.00262, 0.00262, −0.00262, −0.00262, −0.00262, 0.00261, 0.00261, 0.00261, −0.00261, −0.00261, −0.00261, 0.00261, 0.00261, −0.00261, −0.00261, 0.00261, 0.00261, 0.0026, 0.0026, −0.0026, 0.0026, 0.0026, −0.0026, 0.0026, −0.0026, −0.0026, −0.0026, −0.0026, −0.0026, 0.0026, −0.00259, 0.00259, −0.00259, 0.00259, 0.00259, −0.00259, 0.00259, −0.00259, −0.00259, 0.00259, 0.00259, −0.00258, −0.00258, −0.00258, 0.00258, −0.00258, 0.00258, 0.00257, 0.00257, 0.00257, −0.00257, −0.00257, −0.00257, −0.00257, −0.00257, 0.00257, −0.00257, −0.00257, 0.00257, 0.00257, 0.00257, 0.00256, 0.00256, 0.00256, −0.00256, −0.00256, −0.00256, −0.00256, −0.00256, −0.00255, −0.00255, 0.00255, −0.00255, 0.00255, 0.00255, −0.00255, 0.00255, −0.00255, −0.00254, 0.00254, −0.00254, 0.00254, −0.00254, 0.00254, −0.00254, −0.00254, −0.00254, −0.00254, 0.00253, 0.00253, 0.00253, 0.00253, 0.00252, 0.00252, −0.00252, −0.00252, 0.00252, −0.00252, 0.00252, 0.00252, 0.00252, −0.00252, 0.00251, −0.00251, 0.00251, 0.00251, 0.00251, −0.00251, −0.00251, 0.00251, −0.00251, −0.00251, −0.0025, −0.0025, −0.0025, 0.0025, −0.0025, −0.0025, 0.0025, −0.0025, 0.0025, 0.0025, 0.0025, −0.0025, 0.00249, −0.00249, 0.00249, 0.00249, 0.00249, 0.00249, 0.00249, −0.00249, −0.00249, 0.00249, 0.00249, −0.00249, 0.00248, −0.00248, 0.00248, 0.00248, −0.00248, −0.00248, 0.00248, 0.00248, −0.00248, 0.00248, 0.00248, −0.00248, 0.00248, 0.00248, −0.00248, 0.00248, −0.00248, −0.00247, 0.00247, −0.00247, −0.00247, 0.00247, −0.00247, 0.00247, −0.00247, −0.00246, −0.00246, −0.00246, 0.00246, 0.00246, −0.00246, −0.00246, −0.00246, 0.00246, −0.00245, −0.00245, −0.00245, −0.00245, 0.00245, −0.00245, 0.00245, −0.00244, −0.00244, 0.00244, −0.00244, −0.00244, −0.00244, 0.00244, 0.00244, −0.00244, 0.00244, 0.00244, −0.00244, −0.00244, −0.00244, 0.00244, 0.00244, 0.00244, −0.00244, 0.00244, 0.00244, 0.00243, −0.00243, −0.00243, −0.00243, −0.00243, 0.00243, −0.00243, 0.00243, −0.00243, 0.00242, 0.00242, 0.00242, 0.00242, 0.00242, 0.00242, 0.00242, −0.00242, 0.00242, −0.00241, −0.00241, −0.00241, −0.00241, 0.00241, −0.00241, 0.00241, 0.00241, −0.00241, 0.00241, 0.00241, −0.00241, −0.0024, −0.0024, 0.0024, 0.0024, −0.0024, −0.0024, 0.0024, −0.0024, −0.0024, −0.00239, −0.00239, 0.00239, 0.00239, −0.00239, 0.00239, 0.00239, 0.00238, 0.00238, −0.00238, 0.00238, 0.00238, −0.00238, 0.00238, 0.00238, −0.00238, 0.00238, 0.00238, −0.00238, −0.00238, 0.00238, 0.00237, −0.00237, 0.00237, 0.00237, 0.00237, −0.00237, −0.00237, 0.00237, −0.00237, 0.00237, −0.00237, 0.00237, 0.00237, 0.00237, −0.00237, 0.00236, 0.00236, −0.00236, −0.00236, 0.00236, 0.00236, −0.00236, −0.00236, 0.00236, 0.00236, 0.00236, 0.00236, 0.00236, 0.00236, 0.00235, 0.00235, 0.00235, 0.00235, 0.00235, 0.00234, 0.00234, −0.00234, −0.00234, −0.00234, 0.00234, −0.00234, 0.00234, −0.00233, −0.00233, −0.00233, −0.00233, −0.00233, −0.00233, 0.00232, 0.00232, −0.00232, 0.00232, −0.00232, −0.00232, −0.00232, −0.00232, 0.00232, 0.00232, 0.00231, −0.00231, 0.00231, −0.00231, 0.00231, 0.00231, −0.00231, 0.00231, −0.00231, −0.00231, −0.0023, −0.0023, 0.0023, −0.0023, −0.0023, −0.0023, 0.0023, 0.0023, 0.0023, 0.0023, −0.0023, −0.0023, −0.0023, 0.0023, −0.0023, 0.00229, 0.00229, 0.00229, −0.00229, 0.00229, 0.00229, −0.00229, −0.00229, −0.00229, 0.00229, 0.00229, 0.00229, 0.00229, −0.00229, −0.00229, −0.00229, 0.00229, −0.00229, 0.00229, 0.00229, 0.00228, 0.00228, −0.00228, −0.00228, −0.00228, −0.00228, 0.00227, −0.00227, 0.00227, −0.00227, 0.00227, −0.00227, 0.00227, 0.00227, −0.00227, −0.00227, −0.00227, −0.00226, −0.00226, −0.00226, −0.00226, −0.00226, 0.00226, 0.00226, −0.00226, 0.00226, −0.00225, −0.00225, 0.00225, −0.00225, −0.00225, 0.00225, 0.00225, −0.00225, 0.00224, −0.00224, −0.00224, 0.00224, −0.00224, 0.00224, −0.00224, 0.00224, 0.00223, −0.00223, 0.00223, −0.00223, 0.00223, 0.00223, 0.00223, −0.00223, −0.00223, 0.00223, 0.00223, −0.00223, −0.00223, 0.00223, 0.00222, −0.00222, 0.00222, −0.00222, 0.00222, −0.00222, −0.00222, −0.00222, 0.00222, −0.00222, −0.00222, 0.00222, −0.00221, 0.00221, 0.00221, −0.00221, 0.00221, −0.00221, −0.00221, −0.00221, −0.00221, −0.00221, −0.00221, −0.00221, −0.0022, 0.0022, −0.0022, 0.0022, 0.0022, 0.0022, 0.0022, −0.0022, −0.0022, 0.0022, −0.0022, 0.00219, 0.00219, −0.00219, 0.00219, 0.00219, 0.00219, 0.00219, 0.00219, 0.00219, 0.00219, −0.00219, 0.00218, −0.00218, 0.00218, −0.00218, 0.00218, −0.00218, −0.00217, 0.00217, 0.00217, −0.00217, 0.00217, −0.00217, −0.00217, −0.00217, −0.00217, −0.00216, 0.00216, 0.00216, −0.00215, −0.00215, 0.00215, 0.00215, −0.00215, −0.00215, −0.00215, 0.00215, 0.00215, 0.00214, 0.00214, 0.00214, −0.00214, 0.00214, 0.00214, 0.00214, −0.00214, −0.00214, 0.00214, 0.00214, −0.00214, 0.00214, −0.00214, 0.00213, 0.00213, −0.00213, −0.00213, 0.00213, −0.00213, −0.00213, 0.00213, 0.00213, 0.00213, −0.00213, 0.00213, 0.00212, −0.00212, 0.00212, 0.00212, −0.00212, −0.00212, −0.00212, 0.00212, −0.00212, 0.00212, 0.00212, 0.00212, 0.00211, −0.00211, 0.00211, 0.00211, −0.00211, 0.00211, 0.00211, −0.00211, 0.00211, −0.00211, 0.00211, −0.00211, 0.0021, 0.0021, 0.0021, 0.0021, −0.0021, 0.0021, −0.0021, −0.0021, −0.0021, −0.0021, −0.0021, 0.0021, 0.0021, 0.00209, 0.00209, 0.00209, 0.00209, −0.00209, −0.00209, −0.00209, 0.00209, 0.00208, −0.00208, −0.00208, −0.00208, 0.00208, −0.00208, 0.00208, 0.00208, −0.00208, 0.00207, −0.00207, 0.00207, −0.00207, −0.00207, −0.00207, −0.00207, 0.00207, 0.00206, 0.00206, −0.00206, 0.00206, 0.00206, 0.00206, −0.00206, 0.00206, −0.00205, 0.00205, 0.00205, 0.00205, 0.00205, 0.00205, 0.00204, −0.00204, −0.00204, −0.00204, 0.00204, 0.00203, 0.00203, 0.00203, 0.00203, −0.00203, 0.00203, 0.00203, −0.00203, −0.00203, 0.00203, −0.00202, −0.00202, −0.00202, 0.00202, 0.00202, 0.00202, −0.00202, 0.00201, −0.00201, 0.00201, −0.00201, −0.00201, −0.00201, 0.00201, 0.00201, 0.00201, −0.00201, 0.00201, 0.00201, −0.002, 0.002, −0.002, 0.002, −0.002, −0.002, 0.002, −0.002, −0.002, 0.002, −0.002, −0.002, −0.002, 0.002, 0.002, −0.002, −0.002, 0.002, 0.002, −0.002, −0.00199, 0.00199, −0.00199, 0.00199, −0.00199, 0.00199, −0.00199, −0.00199, −0.00199, 0.00199, 0.00199, 0.00199, −0.00199, 0.00199, −0.00198, −0.00198, −0.00198, −0.00198, −0.00198, −0.00198, 0.00198, 0.00198, −0.00198, −0.00198, 0.00198, −0.00198, 0.00198, −0.00198, 0.00197, 0.00197, 0.00197, −0.00197, 0.00197, 0.00197, −0.00197, 0.00197, −0.00197, 0.00197, −0.00197, −0.00197, −0.00197, −0.00197, 0.00197, −0.00197, −0.00196, −0.00196, 0.00196, −0.00196, −0.00196, 0.00196, −0.00196, 0.00196, −0.00196, −0.00196, −0.00195, −0.00195, −0.00195, −0.00195, −0.00195, 0.00195, 0.00195, 0.00195, −0.00195, 0.00195, −0.00195, −0.00194, 0.00194, −0.00194, −0.00194, 0.00194, 0.00194, 0.00194, −0.00194, 0.00194, 0.00194, 0.00194, 0.00193, −0.00193, 0.00193, −0.00193, 0.00193, 0.00193, −0.00193, −0.00193, −0.00193, −0.00192, −0.00192, −0.00192, −0.00192, 0.00192, −0.00192, −0.00192, 0.00192, 0.00192, 0.00191, −0.00191, −0.00191, −0.00191, −0.00191, −0.00191, −0.00191, 0.00191, −0.00191, −0.00191, 0.00191, −0.00191, 0.00191, 0.00191, 0.00191, 0.0019, 0.0019, −0.0019, 0.0019, −0.0019, −0.0019, −0.0019, −0.0019, 0.0019, −0.0019, −0.0019, 0.0019, 0.0019, 0.00189, −0.00189, 0.00189, 0.00189, −0.00189, 0.00189, 0.00189, 0.00189, 0.00189, −0.00189, −0.00189, 0.00189, 0.00188, 0.00188, −0.00188, −0.00188, 0.00188, −0.00188, −0.00188, 0.00188, −0.00188, −0.00188, −0.00188, 0.00188, 0.00188, −0.00188, −0.00187, −0.00187, −0.00187, −0.00187, −0.00187, −0.00187, 0.00187, 0.00187, −0.00187, 0.00187, 0.00186, −0.00186, 0.00186, −0.00186, −0.00186, 0.00186, 0.00186, −0.00186, 0.00186, −0.00186, −0.00185, 0.00185, −0.00185, 0.00185, −0.00185, 0.00185, 0.00185, 0.00185, 0.00185, 0.00185, −0.00185, 0.00185, 0.00185, −0.00185, 0.00184, 0.00184, −0.00184, 0.00184, −0.00184, −0.00184, −0.00183, −0.00183, 0.00183, 0.00183, −0.00183, 0.00183, 0.00183, −0.00183, 0.00183, −0.00182, −0.00182, 0.00182, 0.00182, 0.00182, −0.00182, −0.00182, 0.00182, −0.00182, −0.00182, −0.00182, 0.00182, 0.00182, −0.00181, 0.00181, −0.00181, −0.00181, −0.00181, 0.00181, −0.00181, 0.00181, −0.0018, −0.0018, −0.0018, 0.0018, −0.0018, 0.0018, −0.0018, −0.0018, −0.0018, −0.00179, −0.00179, −0.00179, 0.00179, −0.00179, 0.00179, −0.00179, 0.00179, 0.00179, −0.00179, −0.00179, −0.00179, −0.00179, −0.00179, −0.00178, −0.00178, −0.00178, −0.00178, 0.00178, −0.00178, −0.00178, −0.00177, −0.00177, −0.00177, −0.00177, 0.00177, 0.00177, 0.00177, −0.00177, −0.00177, 0.00177, −0.00177, 0.00177, −0.00177, 0.00176, −0.00176, −0.00176, −0.00176, −0.00176, −0.00176, −0.00175, −0.00175, −0.00175, 0.00175, 0.00175, 0.00175, 0.00175, −0.00175, −0.00175, −0.00175, 0.00175, 0.00175, 0.00174, 0.00174, −0.00174, −0.00174, −0.00174, 0.00174, −0.00174, −0.00174, −0.00174, 0.00174, 0.00174, 0.00173, 0.00173, 0.00173, 0.00173, 0.00173, −0.00173, −0.00173, −0.00173, 0.00172, 0.00172, 0.00172, −0.00172, −0.00172, −0.00172, −0.00172, 0.00172, 0.00171, −0.00171, 0.00171, −0.00171, −0.00171, 0.00171, −0.00171, −0.00171, 0.00171, 0.00171, −0.0017, −0.0017, 0.0017, −0.0017, −0.0017, 0.0017, −0.0017, −0.0017, 0.0017, −0.0017, 0.0017, 0.0017, −0.0017, −0.00169, −0.00169, −0.00169, −0.00169, 0.00169, 0.00169, −0.00169, −0.00169, 0.00169, −0.00169, −0.00168, 0.00168, −0.00168, −0.00168, −0.00168, 0.00168, −0.00168, 0.00167, 0.00167, −0.00167, −0.00167, 0.00167, −0.00167, 0.00167, −0.00167, 0.00167, −0.00166, 0.00166, 0.00166, 0.00166, 0.00166, −0.00166, 0.00166, −0.00166, −0.00166, 0.00166, −0.00166, 0.00166, −0.00166, −0.00166, −0.00165, −0.00165, −0.00165, 0.00165, −0.00165, 0.00165, −0.00165, 0.00165, −0.00165, −0.00164, 0.00164, 0.00164, −0.00164, −0.00164, −0.00164, 0.00164, 0.00164, 0.00164, −0.00163, −0.00163, −0.00163, −0.00163, −0.00163, −0.00163, 0.00163, −0.00163, −0.00162, −0.00162, 0.00162, −0.00162, 0.00162, −0.00162, −0.00162, −0.00162, 0.00162, 0.00162, −0.00162, 0.00162, −0.00161, −0.00161, 0.00161, −0.00161, −0.00161, 0.00161, −0.00161, −0.00161, −0.0016, 0.0016, 0.0016, −0.0016, −0.0016, 0.0016, 0.0016, −0.0016, 0.0016, 0.00159, −0.00159, 0.00159, −0.00159, −0.00159, −0.00159, −0.00159, −0.00159, −0.00159, −0.00158, −0.00158, −0.00158, 0.00158, −0.00158, −0.00158, −0.00158, 0.00158, −0.00158, −0.00158, 0.00157, 0.00157, 0.00157, −0.00157, 0.00157, −0.00157, −0.00157, 0.00157, −0.00157, 0.00157, −0.00156, −0.00156, −0.00156, 0.00156, −0.00155, 0.00155, −0.00155, 0.00155, −0.00155, −0.00154, 0.00154, −0.00154, −0.00154, 0.00154, 0.00154, −0.00154, 0.00154, −0.00154, −0.00154, −0.00154, 0.00154, 0.00154, −0.00154, −0.00153, −0.00153, −0.00153, −0.00153, −0.00153, 0.00153, −0.00153, 0.00153, −0.00153, 0.00153, 0.00153, 0.00153, −0.00153, 0.00153, −0.00153, 0.00153, −0.00153, −0.00152, −0.00152, −0.00152, 0.00152, 0.00152, 0.00152, 0.00152, 0.00152, 0.00152, 0.00152, −0.00152, −0.00152, 0.00152, −0.00151, −0.00151, 0.00151, −0.00151, −0.00151, −0.00151, 0.00151, 0.00151, 0.00151, −0.00151, 0.00151, −0.00151, 0.0015, −0.0015, −0.0015, 0.0015, 0.0015, 0.0015, 0.0015, 0.0015, −0.0015, −0.0015, −0.0015, 0.0015, −0.0015, 0.0015, 0.0015, −0.00149, 0.00149, 0.00149, −0.00149, 0.00149, −0.00149, −0.00149, −0.00149, 0.00149, −0.00149, 0.00149, −0.00149, −0.00148, 0.00148, 0.00148, −0.00148, −0.00148, 0.00147, −0.00147, −0.00147, 0.00147, 0.00147, 0.00147, 0.00147, −0.00147, −0.00146, −0.00146, 0.00146, −0.00146, 0.00146, 0.00146, −0.00146, −0.00146, 0.00145, 0.00145, −0.00145, 0.00145, 0.00145, −0.00144, −0.00144, 0.00144, −0.00144, 0.00144, −0.00144, 0.00144, −0.00144, 0.00144, −0.00144, 0.00144, 0.00144, −0.00143, 0.00143, 0.00143, 0.00143, −0.00143, 0.00143, −0.00143, 0.00143, −0.00143, 0.00143, 0.00143, 0.00143, 0.00143, 0.00142, 0.00142, −0.00142, −0.00142, 0.00142, 0.00142, 0.00142, −0.00142, −0.00142, 0.00142, −0.00142, −0.00142, −0.00142, 0.00142, −0.00142, 0.00141, −0.00141, 0.00141, −0.00141, 0.00141, 0.00141, −0.00141, −0.00141, 0.00141, −0.00141, 0.0014, −0.0014, −0.0014, −0.0014, −0.0014, −0.0014, −0.0014, 0.0014, −0.0014, 0.0014, −0.0014, −0.0014, 0.0014, −0.00139, −0.00139, 0.00139, −0.00139, −0.00139, 0.00139, 0.00139, −0.00138, −0.00138, 0.00138, −0.00138, 0.00138, 0.00138, −0.00138, 0.00138, −0.00138, −0.00138, 0.00138, 0.00138, 0.00138, 0.00138, −0.00138, 0.00137, −0.00137, 0.00137, 0.00137, 0.00137, 0.00137, 0.00137, 0.00137, 0.00137, 0.00137, 0.00137, −0.00137, −0.00137, −0.00136, 0.00136, −0.00136, −0.00136, −0.00136, −0.00136, −0.00136, 0.00136, −0.00136, −0.00136, −0.00136, −0.00136, 0.00136, 0.00135, 0.00135, 0.00135, 0.00135, 0.00135, −0.00135, −0.00135, −0.00135, 0.00135, 0.00135, 0.00135, −0.00135, −0.00134, −0.00134, −0.00134, −0.00134, −0.00134, −0.00134, 0.00133, 0.00133, −0.00133, 0.00133, −0.00133, −0.00133, −0.00133, 0.00133, 0.00132, 0.00132, 0.00132, 0.00132, −0.00132, 0.00132, 0.00132, 0.00132, 0.00132, −0.00132, 0.00132, −0.00132, −0.00132, 0.00132, −0.00132, 0.00132, 0.00131, 0.00131, 0.00131, 0.00131, −0.00131, 0.00131, 0.00131, 0.00131, 0.00131, −0.00131, −0.00131, −0.00131, −0.00131, 0.00131, 0.00131, 0.00131, 0.0013, −0.0013, 0.0013, −0.0013, −0.0013, 0.0013, 0.0013, −0.0013, −0.0013, −0.0013, 0.00129, −0.00129, −0.00129, −0.00129, −0.00128, −0.00128, 0.00128, −0.00128, 0.00128, −0.00128, 0.00128, −0.00128, −0.00128, −0.00128, −0.00128, −0.00128, 0.00128, 0.00127, 0.00127, 0.00127, 0.00127, −0.00127, 0.00127, 0.00126, −0.00126, 0.00126, 0.00126, 0.00126, −0.00126, 0.00126, 0.00126, −0.00126, 0.00126, 0.00126, 0.00125, 0.00125, −0.00125, 0.00125, 0.00125, 0.00125, −0.00125, 0.00125, −0.00124, −0.00124, −0.00124, 0.00124, −0.00124, −0.00124, −0.00124, 0.00124, 0.00124, −0.00124, 0.00124, −0.00124, −0.00123, −0.00123, 0.00123, 0.00123, 0.00123, 0.00123, −0.00123, −0.00123, 0.00122, −0.00122, 0.00122, −0.00122, −0.00122, 0.00122, 0.00121, 0.00121, −0.00121, 0.00121, 0.00121, −0.00121, 0.00121, 0.00121, 0.00121, 0.00121, 0.0012, 0.0012, −0.0012, 0.0012, −0.00119, 0.00119, 0.00119, −0.00119, −0.00119, 0.00119, −0.00119, 0.00119, −0.00119, −0.00119, −0.00119, −0.00118, −0.00118, −0.00118, −0.00118, 0.00118, −0.00118, −0.00118, 0.00118, 0.00117, −0.00117, 0.00117, 0.00117, 0.00117, −0.00117, −0.00117, −0.00117, −0.00117, 0.00117, −0.00117, 0.00117, 0.00117, 0.00117, −0.00116, −0.00116, 0.00116, 0.00116, −0.00116, −0.00116, 0.00116, 0.00116, 0.00116, 0.00115, −0.00115, 0.00115, −0.00115, 0.00115, 0.00115, −0.00115, 0.00115, −0.00115, 0.00115, 0.00115, 0.00115, −0.00115, −0.00114, 0.00114, −0.00114, −0.00114, 0.00114, −0.00114, 0.00114, 0.00114, 0.00113, −0.00113, 0.00113, −0.00113, −0.00113, −0.00113, −0.00113, −0.00113, 0.00113, −0.00113, −0.00113, 0.00113, −0.00113, −0.00112, 0.00112, 0.00112, −0.00112, 0.00112, −0.00112, 0.00112, −0.00112, 0.00112, −0.00112, −0.00112, 0.00111, −0.00111, 0.00111, 0.00111, −0.00111, −0.00111, 0.00111, 0.00111, −0.0011, 0.0011, −0.0011, 0.0011, 0.0011, −0.0011, −0.0011, 0.0011, −0.00109, 0.00109, 0.00109, 0.00109, −0.00109, 0.00109, −0.00109, 0.00109, −0.00109, 0.00109, −0.00109, 0.00109, 0.00109, 0.00109, 0.00109, 0.00109, 0.00108, −0.00108, −0.00108, 0.00108, 0.00108, −0.00108, −0.00108, −0.00108, −0.00108, 0.00108, 0.00108, −0.00108, −0.00107, 0.00107, 0.00107, −0.00107, 0.00107, −0.00107, −0.00107, −0.00107, −0.00107, 0.00107, −0.00107, −0.00106, 0.00106, −0.00106, 0.00106, 0.00106, −0.00106, −0.00106, 0.00106, 0.00106, −0.00106, 0.00106, −0.00106, −0.00106, −0.00105, −0.00105, −0.00105, −0.00105, −0.00105, 0.00105, −0.00105, 0.00105, −0.00105, −0.00105, −0.00105, −0.00105, −0.00104, −0.00104, 0.00104, −0.00104, 0.00104, 0.00104, −0.00104, −0.00104, 0.00104, 0.00104, −0.00104, −0.00104, 0.00104, 0.00104, −0.00104, −0.00104, 0.00103, 0.00103, −0.00103, 0.00103, 0.00103, 0.00103, 0.00103, −0.00103, −0.00103, −0.00103, 0.00103, 0.00103, −0.00103, 0.00103, −0.00103, 0.00103, 0.00102, 0.00102, −0.00102, −0.00102, 0.00102, 0.00102, 0.00102, 0.00102, −0.00101, 0.00101, −0.00101, −0.00101, −0.00101, 0.00101, 0.00101, 0.001, −0.001, −0.001, −0.001, −0.001, 0.001, 0.001, 0.001, −0.001, 0.001, 0.001, 0.001, 0.001, 0.001, −0.001, 0.001, 0.001, −0.001, 0.00099, 0.00099, 0.00099, 0.00099, −0.00099, −0.00099, −0.00099, −0.00099, −0.00099, 0.00099, 0.00099, 0.00099, 0.00099, −0.00099, −0.00099, −0.00099, −0.00099, 0.00099, −0.00098, −0.00098, −0.00098, −0.00098, 0.00098, −0.00098, 0.00098, 0.00098, −0.00098, 0.00098, 0.00098, −0.00098, 0.00098, 0.00098, 0.00097, 0.00097, −0.00097, 0.00097, −0.00097, 0.00097, −0.00097, −0.00097, 0.00097, −0.00097, 0.00097, 0.00097, 0.00096, 0.00096, −0.00096, 0.00096, 0.00096, 0.00096, −0.00095, −0.00095, −0.00095, 0.00095, 0.00095, −0.00095, 0.00095, 0.00095, 0.00095, 0.00095, 0.00094, 0.00094, 0.00094, −0.00094, −0.00094, −0.00094, −0.00094, 0.00094, −0.00093, 0.00093, −0.00093, −0.00093, 0.00093, 0.00093, 0.00093, −0.00093, −0.00093, −0.00093, 0.00093, −0.00093, 0.00093, −0.00093, −0.00093, −0.00093, −0.00093, −0.00093, 0.00093, −0.00093, −0.00092, 0.00092, 0.00092, −0.00092, 0.00092, 0.00092, 0.00092, −0.00092, −0.00092, −0.00092, −0.00092, −0.00091, −0.00091, −0.00091, 0.00091, −0.00091, 0.00091, −0.00091, 0.00091, 0.00091, 0.00091, 0.00091, −0.00091, 0.00091, 9.00E-04, −9.00E-04, −9.00E-04, −9.00E-04, 9.00E-04, 9.00E-04, 9.00E-04, −9.00E-04, −9.00E-04, 9.00E-04, −9.00E-04, 0.00089, 0.00089, 0.00089, 0.00089, 0.00089, −0.00089, 0.00089, 0.00089, −0.00089, −0.00089, −0.00089, 0.00089, −0.00089, −0.00088, −0.00088, 0.00088, −0.00088, −0.00088, 0.00088, 0.00088, −0.00088, 0.00088, −0.00088, 0.00088, 0.00088, −0.00088, 0.00088, −0.00088, −0.00088, −0.00088, 0.00088, −0.00088, 0.00087, 0.00087, −0.00087, −0.00087, −0.00087, −0.00087, −0.00087, −0.00087, 0.00087, 0.00087, 0.00087, −0.00087, 0.00087, 0.00087, 0.00086, 0.00086, 0.00086, 0.00086, −0.00086, 0.00086, −0.00086, −0.00086, 0.00085, 0.00085, 0.00085, −0.00085, −0.00085, 0.00085, 0.00085, 0.00085, −0.00085, 0.00084, −0.00084, 0.00084, −0.00084, 0.00084, 0.00084, 0.00084, 0.00084, −0.00083, −0.00083, −0.00083, 0.00083, −0.00083, 0.00083, 0.00082, 0.00082, 0.00082, 0.00082, −0.00082, 0.00082, −0.00082, 0.00082, −0.00081, 0.00081, 0.00081, −0.00081, 0.00081, −8.00E-04, 8.00E-04, −8.00E-04, −8.00E-04, 8.00E-04, 8.00E-04, 8.00E-04, 8.00E-04, −8.00E-04, 8.00E-04, 8.00E-04, 8.00E-04, 8.00E-04, 8.00E-04, −8.00E-04, −8.00E-04, 8.00E-04, 0.00079, −0.00079, −0.00079, 0.00079, −0.00079, 0.00079, 0.00079, −0.00079, −0.00079, −0.00079, 0.00079, 0.00079, −0.00079, −0.00079, 0.00078, 0.00078, 0.00078, −0.00077, 0.00077, −0.00077, −0.00077, 0.00077, 0.00077, −0.00077, −0.00077, 0.00077, −0.00077, −0.00077, −0.00076, 0.00076, 0.00076, −0.00076, −0.00076, 0.00076, −0.00076, 0.00075, −0.00075, 0.00075, −0.00075, −0.00075, −0.00075, −0.00075, 0.00075, 0.00074, −0.00074, 0.00074, 0.00074, −0.00074, 0.00074, −0.00074, −0.00074, −0.00074, 0.00074, −0.00074, −0.00074, 0.00074, 0.00073, 0.00073, −0.00073, 0.00073, −0.00073, 0.00073, −0.00073, −0.00073, 0.00073, 0.00073, −0.00073, −0.00073, 0.00073, −0.00073, 0.00073, −0.00072, 0.00072, −0.00072, −0.00072, −0.00072, 0.00072, −0.00072, 0.00072, −0.00072, 0.00072, −0.00072, 0.00072, −0.00071, −0.00071, 0.00071, 0.00071, −0.00071, −0.00071, −0.00071, −0.00071, −0.00071, −0.00071, 0.00071, 0.00071, −0.00071, 0.00071, 7.00E-04, −7.00E-04, 7.00E-04, −7.00E-04, 7.00E-04, −7.00E-04, 7.00E-04, 7.00E-04, 7.00E-04, −7.00E-04, 0.00069, −0.00069, 0.00069, 0.00069, 0.00069, 0.00069, 0.00069, 0.00069, 0.00069, 0.00069, 0.00068, 0.00068, −0.00068, −0.00068, 0.00068, 0.00068, −0.00067, −0.00067, −0.00067, 0.00067, 0.00067, 0.00067, 0.00067, 0.00067, 0.00067, −0.00066, −0.00066, −0.00066, 0.00066, 0.00066, −0.00066, 0.00066, 0.00066, 0.00066, 0.00066, −0.00066, −0.00065, −0.00065, −0.00065, 0.00065, 0.00065, 0.00065, 0.00065, 0.00065, 0.00064, −0.00064, −0.00064, 0.00064, 0.00064, 0.00064, −0.00064, 0.00064, −0.00064, −0.00064, −0.00064, −0.00063, 0.00063, −0.00063, −0.00063, −0.00063, 0.00063, −0.00063, 0.00063, 0.00063, −0.00063, −0.00063, 0.00063, 0.00063, 0.00063, 0.00063, 0.00063, 0.00062, −0.00062, −0.00062, 0.00062, −0.00062, −0.00062, −0.00062, 0.00062, 0.00061, −0.00061, −0.00061, 0.00061, 0.00061, 0.00061, 0.00061, 0.00061, 0.00061, 6.00E-04, 6.00E-04, −6.00E-04, −6.00E-04, −6.00E-04, −6.00E-04, −6.00E-04, −6.00E-04, 0.00059, −0.00059, −0.00059, −0.00059, −0.00059, −0.00059, −0.00059, 0.00059, −0.00059, −0.00059, 0.00059, −0.00059, 0.00058, 0.00058, 0.00058, 0.00058, −0.00058, −0.00058, −0.00058, 0.00058, −0.00058, 0.00058, −0.00058, −0.00057, 0.00057, −0.00057, −0.00057, −0.00057, −0.00057, 0.00057, −0.00057, −0.00057, −0.00057, 0.00057, −0.00056, −0.00056, −0.00056, −0.00056, −0.00056, 0.00056, 0.00056, 0.00056, −0.00056, 0.00056, −0.00056, −0.00055, 0.00055, 0.00055, −0.00055, −0.00055, −0.00055, −0.00055, −0.00054, −0.00054, −0.00054, −0.00054, 0.00054, 0.00054, −0.00054, −0.00054, −0.00053, −0.00053, −0.00053, 0.00053, −0.00053, 0.00053, 0.00053, 0.00053, −0.00053, −0.00053, −0.00053, −0.00053, 0.00053, −0.00053, 0.00052, 0.00052, 0.00052, 0.00052, −0.00052, 0.00052, −0.00052, 0.00051, 0.00051, 0.00051, 0.00051, 0.00051, 0.00051, −0.00051, −0.00051, 0.00051, 0.00051, −0.00051, −0.00051, 5.00E-04, −5.00E-04, 5.00E-04, −5.00E-04, −5.00E-04, −5.00E-04, 0.00049, −0.00049, 0.00049, −0.00049, −0.00049, −0.00049, 0.00049, −0.00048, −0.00048, −0.00048, 0.00048, 0.00048, 0.00048, −0.00048, −0.00048, 0.00047, 0.00047, −0.00047, −0.00047, 0.00047, 0.00047, 0.00047, −0.00047, −0.00047, −0.00047, 0.00047, −0.00047, −0.00046, 0.00046, 0.00046, −0.00046, −0.00046, −0.00045, −0.00045, 0.00045, 0.00045, 0.00045, 0.00045, −0.00045, −0.00045, 0.00045, −0.00045, 0.00044, 0.00044, −0.00044, 0.00044, 0.00044, −0.00044, −0.00044, 0.00044, −0.00044, −0.00043, −0.00043, −0.00043, −0.00043, 0.00043, 0.00043, 0.00043, −0.00043, 0.00043, −0.00043, 0.00043, −0.00043, −0.00043, 0.00042, 0.00042, 0.00042, 0.00042, −0.00042, −0.00042, 0.00042, 0.00042, −0.00042, 0.00042, −0.00042, −0.00042, −0.00042, −0.00042, −0.00042, −0.00041, −0.00041, 0.00041, 0.00041, −0.00041, 0.00041, 0.00041, −0.00041, −0.00041, 0.00041, 4.00E-04, 4.00E-04, 4.00E-04, −4.00E-04, −4.00E-04, 4.00E-04, −4.00E-04, 4.00E-04, −4.00E-04, 4.00E-04, 4.00E-04, −0.00039, 0.00039, 0.00039, 0.00039, −0.00039, 0.00039, −0.00039, 0.00039, −0.00039, −0.00039, 0.00039, −0.00039, −0.00039, −0.00038, −0.00038, 0.00038, −0.00038, −0.00038, 0.00038, 0.00038, −0.00038, 0.00038, 0.00037, −0.00037, 0.00037, 0.00037, −0.00037, 0.00037, −0.00037, −0.00037, 0.00036, 0.00036, 0.00036, −0.00036, 0.00036, 0.00036, 0.00036, −0.00036, −0.00036, 0.00036, −0.00036, 0.00036, −0.00035, 0.00035, −0.00035, 0.00035, −0.00035, −0.00035, 0.00035, −0.00035, −0.00035, 0.00035, 0.00035, 0.00035, −0.00035, −0.00035, 0.00035, 0.00035, 0.00035, 0.00034, 0.00034, 0.00034, −0.00034, 0.00034, 0.00034, 0.00034, −0.00034, −0.00034, 0.00034, −0.00034, −0.00034, 0.00034, 0.00033, −0.00033, 0.00033, 0.00033, −0.00033, 0.00033, −0.00033, 0.00033, −0.00032, −0.00032, 0.00032, 0.00032, 0.00032, −0.00032, 0.00032, 0.00032, −0.00032, −0.00032, 0.00032, −0.00032, 0.00031, 0.00031, −0.00031, 0.00031, −0.00031, −0.00031, 0.00031, 3.00E-04, −3.00E-04, −3.00E-04, 3.00E-04, 3.00E-04, 3.00E-04, 3.00E-04, −3.00E-04, −0.00029, −0.00029, 0.00029, −0.00029, 0.00028, 0.00028, 0.00028, 0.00028, −0.00028, 0.00028, −0.00028, −0.00028, −0.00028, −0.00028, 0.00028, 0.00028, −0.00028, −0.00028, −0.00027, 0.00027, 0.00027, −0.00027, −0.00027, −0.00027, −0.00027, −0.00027, −0.00027, 0.00027, 0.00027, 0.00027, 0.00027, −0.00027, 0.00027, 0.00026, −0.00026, −0.00026, 0.00026, −0.00026, −0.00026, −0.00026, −0.00026, 0.00026, −0.00026, −0.00025, −0.00025, −0.00025, −0.00025, −0.00025, −0.00025, −0.00025, −0.00025, −0.00025, −0.00025, 0.00025, 0.00025, 0.00025, 0.00024, 0.00024, −0.00024, 0.00024, 0.00024, −0.00024, 0.00024, −0.00024, −0.00024, 0.00024, −0.00024, −0.00024, 0.00024, −0.00024, 0.00023, −0.00023, −0.00023, −0.00023, −0.00023, −0.00023, −0.00023, −0.00023, 0.00023, −0.00023, 0.00023, −0.00023, −0.00023, 0.00023, 0.00022, −0.00022, 0.00022, 0.00022, −0.00022, 0.00022, −0.00022, 0.00022, 0.00022, 0.00022, 0.00022, −0.00022, −0.00021, −0.00021, −0.00021, 0.00021, 0.00021, −0.00021, 0.00021, −0.00021, −0.00021, −0.00021, 2.00E-04, 2.00E-04, −2.00E-04, 2.00E-04, −2.00E-04, −2.00E-04, 2.00E-04, −2.00E-04, 2.00E-04, 2.00E-04, −2.00E-04, 0.00019, 0.00019, −0.00019, −0.00019, 0.00019, 0.00019, 0.00019, 0.00019, 0.00019, −0.00019, 0.00019, −0.00019, 0.00019, −0.00018, −0.00018, −0.00018, −0.00018, −0.00018, −0.00018, −0.00018, −0.00018, 0.00018, 0.00018, −0.00018, −0.00018, −0.00018, −0.00018, −0.00018, −0.00017, −0.00017, −0.00017, −0.00017, −0.00017, 0.00017, 0.00017, −0.00017, 0.00016, −0.00016, −0.00016, −0.00016, 0.00016, −0.00016, −0.00016, 0.00016, −0.00016, 0.00015, 0.00015, 0.00015, 0.00015, 0.00015, −0.00015, 0.00015, −0.00015, −0.00015, 0.00014, −0.00014, −0.00014, −0.00014, 0.00014, 0.00014, −0.00013, 0.00013, 0.00013, 0.00013, −0.00013, −0.00013, 0.00013, 0.00013, −0.00013, −0.00013, −0.00012, 0.00012, −0.00012, −0.00012, 0.00012, 0.00012, −0.00012, −0.00012, 0.00012, 0.00012, −0.00012, −0.00012, −0.00011, 0.00011, 0.00011, −0.00011, −0.00011, −0.00011, 0.00011, −0.00011, −0.00011, −0.00011, −1.00E-04, −1.00E-04, −1.00E-04, 1.00E-04, 1.00E-04, 1.00E-04, 1.00E-04, 1.00E-04, 1.00E-04, −1.00E-04, −9.00E-05, −9.00E-05, 9.00E-05, −9.00E-05, 9.00E-05, −9.00E-05, −9.00E-05, −9.00E-05, 9.00E-05, 9.00E-05, 9.00E-05, −9.00E-05, 8.00E-05, −8.00E-05, 8.00E-05, 8.00E-05, 8.00E-05, 8.00E-05, −8.00E-05, 8.00E-05, 8.00E-05, 8.00E-05, 7.00E-05, 7.00E-05, 7.00E-05, −7.00E-05, 7.00E-05, −7.00E-05, 7.00E-05, 7.00E-05, 7.00E-05, −7.00E-05, −6.00E-05, −6.00E-05, 6.00E-05, −6.00E-05, 6.00E-05, 6.00E-05, 6.00E-05, −6.00E-05, 6.00E-05, 6.00E-05, 6.00E-05, −6.00E-05, 5.00E-05, −5.00E-05, 5.00E-05, 5.00E-05, 5.00E-05, −5.00E-05, −5.00E-05, −5.00E-05, 4.00E-05, −4.00E-05, 4.00E-05, 4.00E-05, −4.00E-05, −4.00E-05, 4.00E-05, −4.00E-05, −4.00E-05, 4.00E-05, −3.00E-05, −3.00E-05, −3.00E-05, 3.00E-05, −3.00E-05, −3.00E-05, −3.00E-05, 2.00E-05, 2.00E-05, −2.00E-05, 2.00E-05, 2.00E-05, 2.00E-05, 1.00E-05, −1.00E-05, 1.00E-05, 1.00E-05, 1.00E-05, −1.00E-05, 1.00E-05, −1.00E-05, −1.00E-05, −1.00E-05, 1.00E-05, 0, 0, 0, 0, and 0.

The predicting the presence, absence or development of cancer in an individual may particularly involve a threshold cancer index value being applied in order to assess or stratify an individual has having or not having cancer or of having a high or low risk of cancer development.

In any of the assays described herein, wherein when the cancer index value for the individual is about −0.278 or more, the individual is assessed as having cancer or as having a high risk of cancer development, or wherein when the cancer index value for the individual is less than about −0.278, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-BC-Index. The panel of one or more CpGs used to derive the cancer index value may particularly comprise:
a. at least 500 of the CpGs identified at nucleotide positions 61 to 62 in SEQ ID NOs 1 to 29,000, and wherein the sensitivity is at least 63% and the specificity is at least 21%;
b. at least the CpGs defined by SEQ ID NOs 1 to 500 and identified at nucleotide positions 61 to 62, and wherein the sensitivity is at least 87% and specificity is at least 50%;
c. at least the CpGs defined by SEQ ID NOs 1 to 1000 and identified at nucleotide positions 61 to 62, and wherein the sensitivity is at least 89% and specificity is at least 49%; or
d. at least the CpGs defined by SEQ ID NOs 1 to 2000 and identified at nucleotide positions 61 to 62, and wherein the sensitivity is at least 89% and specificity is at least 48%.

In any of the assays described herein, wherein when the cancer index value for the individual is about −0.278 or more, the individual is assessed as having cancer or as having a high risk of cancer development, or wherein when the cancer index value for the individual is less than about −0.278, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-BC-Index. The panel of one or more CpGs used to derive the cancer index value may particularly comprise:
1. at least the CpGs defined by SEQ ID NOs 1 to 500 and identified at nucleotide positions 61 to 62, and wherein the sensitivity is at least 87% and specificity is at least 50%;
2. at least the CpGs defined by SEQ ID NOs 501 to 100 and identified at nucleotide positions 61 to 62, and wherein the sensitivity is at least 87% and specificity is at least 50%; or
3. at least the CpGs defined by SEQ ID NOs 1001 to 1500 and identified at nucleotide positions 61 to 62, and wherein the sensitivity is at least 85% and specificity is at least 50%;
4. at least the CpGs defined by SEQ ID NOs 1501 to 2000 and identified at nucleotide positions 61 to 62, and wherein the sensitivity is at least 82% and specificity is at least 45%;
5. at least the CpGs defined by SEQ ID NOs 2001 to 2500 and identified at nucleotide positions 61 to 62, and wherein the sensitivity is at least 86% and specificity is at least 49%;
6. at least the CpGs defined by SEQ ID NOs 2501 to 3000 and identified at nucleotide positions 61 to 62, and wherein the sensitivity is at least 83% and specificity is at least 47%;
7. at least the CpGs defined by SEQ ID NOs 3001 to 3500 and identified at nucleotide positions 61 to 62, and wherein the sensitivity is at least 82% and specificity is at least 53%;
8. at least the CpGs defined by SEQ ID NOs 3501 to 4000 and identified at nucleotide positions 61 to 62, and wherein the sensitivity is at least 78% and specificity is at least 48%;
9. at least the CpGs defined by SEQ ID NOs 4001 to 4500 and identified at nucleotide positions 61 to 62, and wherein the sensitivity is at least 82% and specificity is at least 48%;
10. at least the CpGs defined by SEQ ID NOs 4501 to 5000 and identified at nucleotide positions 61 to 62, and wherein the sensitivity is at least 80% and specificity is at least 51%;
11. at least the CpGs defined by SEQ ID NOs 5001 to 5500 and identified at nucleotide positions 61 to 62, and wherein the sensitivity is at least 87% and specificity is at least 51%;
12. at least the CpGs defined by SEQ ID NOs 5501 to 6000 and identified at nucleotide positions 61 to 62, and wherein the sensitivity is at least 75% and specificity is at least 51%;
13. at least the CpGs defined by SEQ ID NOs 6001 to 6500 and identified at nucleotide positions 61 to 62, and wherein the sensitivity is at least 77% and specificity is at least 50%;
14. at least the CpGs defined by SEQ ID NOs 6501 to 7000 and identified at nucleotide positions 61 to 62, and wherein the sensitivity is at least 77% and specificity is at least 49%;
15. at least the CpGs defined by SEQ ID NOs 7001 to 7500 and identified at nucleotide positions 61 to 62, and wherein the sensitivity is at least 80% and specificity is at least 51%;
16. at least the CpGs defined by SEQ ID NOs 7501 to 8000 and identified at nucleotide positions 61 to 62, and wherein the sensitivity is at least 77% and specificity is at least 49%;
17. at least the CpGs defined by SEQ ID NOs 8001 to 8500 and identified at nucleotide positions 61 to 62, and wherein the sensitivity is at least 79% and specificity is at least 50%;
18. at least the CpGs defined by SEQ ID NOs 8501 to 9000 and identified at nucleotide positions 61 to 62, and wherein the sensitivity is at least 79% and specificity is at least 51%;
19. at least the CpGs defined by SEQ ID NOs 9001 to 9500 and identified at nucleotide positions 61 to 62, and wherein the sensitivity is at least 78% and specificity is at least 54%;
20. at least the CpGs defined by SEQ ID NOs 9501 to 10000 and identified at nucleotide positions 61 to 62, and wherein the sensitivity is at least 71% and specificity is at least 52%;
21. at least the CpGs defined by SEQ ID NOs 10001 to 10500 and identified at nucleotide positions 61 to 62, and wherein the sensitivity is at least 71% and specificity is at least 53%;
22. at least the CpGs defined by SEQ ID NOs 10501 to 11000 and identified at nucleotide positions 61 to 62, and wherein the sensitivity is at least 73% and specificity is at least 52%;
23. at least the CpGs defined by SEQ ID NOs 11001 to 11500 and identified at nucleotide positions 61 to 62, and wherein the sensitivity is at least 69% and specificity is at least 54%;
24. at least the CpGs defined by SEQ ID NOs 11501 to 12000 and identified at nucleotide positions 61 to 62, and wherein the sensitivity is at least 64% and specificity is at least 54%;
25. at least the CpGs defined by SEQ ID NOs 12001 to 12500 and identified at nucleotide positions 61 to 62, and wherein the sensitivity is at least 70% and specificity is at least 50%;
26. at least the CpGs defined by SEQ ID NOs 12501 to 13000 and identified at nucleotide positions 61 to 62, and wherein the sensitivity is at least 66% and specificity is at least 55%;
27. at least the CpGs defined by SEQ ID NOs 13001 to 13500 and identified at nucleotide positions 61 to 62, and wherein the sensitivity is at least 72% and specificity is at least 55%;
28. at least the CpGs defined by SEQ ID NOs 13501 to 14000 and identified at nucleotide positions 61 to 62, and wherein the sensitivity is at least 69% and specificity is at least 52%;
29. at least the CpGs defined by SEQ ID NOs 14001 to 14500 and identified at nucleotide positions 61 to 62, and wherein the sensitivity is at least 64% and specificity is at least 55%;
30. at least the CpGs defined by SEQ ID NOs 14501 to 15000 and identified at nucleotide positions 61 to 62, and wherein the sensitivity is at least 62% and specificity is at least 54%;
31. at least the CpGs defined by SEQ ID NOs 15001 to 15500 and identified at nucleotide positions 61 to 62, and wherein the sensitivity is at least 75% and specificity is at least 52%;
32. at least the CpGs defined by SEQ ID NOs 15501 to 16000 and identified at nucleotide positions 61 to 62, and wherein the sensitivity is at least 56% and specificity is at least 24%;
33. at least the CpGs defined by SEQ ID NOs 16001 to 16500 and identified at nucleotide positions 61 to 62, and wherein the sensitivity is at least 62% and specificity is at least 25%;
34. at least the CpGs defined by SEQ ID NOs 16501 to 17000 and identified at nucleotide positions 61 to 62, and wherein the sensitivity is at least 58% and specificity is at least 23%;
35. at least the CpGs defined by SEQ ID NOs 17001 to 17500 and identified at nucleotide positions 61 to 62, and wherein the sensitivity is at least 66% and specificity is at least 23%;
36. at least the CpGs defined by SEQ ID NOs 17501 to 18000 and identified at nucleotide positions 61 to 62, and wherein the sensitivity is at least 59% and specificity is at least 24%;
37. at least the CpGs defined by SEQ ID NOs 18001 to 18500 and identified at nucleotide positions 61 to 62, and wherein the sensitivity is at least 65% and specificity is at least 23%;
38. at least the CpGs defined by SEQ ID NOs 18501 to 19000 and identified at nucleotide positions 61 to 62, and wherein the sensitivity is at least 62% and specificity is at least 22%;
39. at least the CpGs defined by SEQ ID NOs 19001 to 19500 and identified at nucleotide positions 61 to 62, and wherein the sensitivity is at least 61% and specificity is at least 23%;
40. at least the CpGs defined by SEQ ID NOs 19501 to 20000 and identified at nucleotide positions 61 to 62, and wherein the sensitivity is at least 60% and specificity is at least 22%;
41. at least the CpGs defined by SEQ ID NOs 20001 to 20500 and identified at nucleotide positions 61 to 62, and wherein the sensitivity is at least 62% and specificity is at least 21%;
42. at least the CpGs defined by SEQ ID NOs 20501 to 21000 and identified at nucleotide positions 61 to 62, and wherein the sensitivity is at least 62% and specificity is at least 25%;
43. at least the CpGs defined by SEQ ID NOs 21001 to 21500 and identified at nucleotide positions 61 to 62, and wherein the sensitivity is at least 62% and specificity is at least 21%;
44. at least the CpGs defined by SEQ ID NOs 21501 to 22000 and identified at nucleotide positions 61 to 62, and wherein the sensitivity is at least 62% and specificity is at least 22%;
45. at least the CpGs defined by SEQ ID NOs 22001 to 22500 and identified at nucleotide positions 61 to 62, and wherein the sensitivity is at least 58% and specificity is at least 22%;
46. at least the CpGs defined by SEQ ID NOs 22501 to 23000 and identified at nucleotide positions 61 to 62, and wherein the sensitivity is at least 62% and specificity is at least 24%;
47. at least the CpGs defined by SEQ ID NOs 23001 to 23500 and identified at nucleotide positions 61 to 62, and wherein the sensitivity is at least 64% and specificity is at least 21%;
48. at least the CpGs defined by SEQ ID NOs 23501 to 24000 and identified at nucleotide positions 61 to 62, and wherein the sensitivity is at least 63% and specificity is at least 23%;
49. at least the CpGs defined by SEQ ID NOs 24001 to 24500 and identified at nucleotide positions 61 to 62, and wherein the sensitivity is at least 62% and specificity is at least 22%;
50. at least the CpGs defined by SEQ ID NOs 24501 to 25000 and identified at nucleotide positions 61 to 62, and wherein the sensitivity is at least 69% and specificity is at least 24%;
51. at least the CpGs defined by SEQ ID NOs 25001 to 25500 and identified at nucleotide positions 61 to 62, and wherein the sensitivity is at least 63% and specificity is at least 19%;
52. at least the CpGs defined by SEQ ID NOs 25501 to 26000 and identified at nucleotide positions 61 to 62, and wherein the sensitivity is at least 62% and specificity is at least 23%;
53. at least the CpGs defined by SEQ ID NOs 26001 to 26500 and identified at nucleotide positions 61 to 62, and wherein the sensitivity is at least 66% and specificity is at least 24%;
54. at least the CpGs defined by SEQ ID NOs 26501 to 27000 and identified at nucleotide positions 61 to 62, and wherein the sensitivity is at least 62% and specificity is at least 23%;
55. at least the CpGs defined by SEQ ID NOs 27001 to 27500 and identified at nucleotide positions 61 to 62, and wherein the sensitivity is at least 68% and specificity is at least 22%;
56. at least the CpGs defined by SEQ ID NOs 27501 to 28000 and identified at nucleotide positions 61 to 62, and wherein the sensitivity is at least 63% and specificity is at least 23%;
57. at least the CpGs defined by SEQ ID NOs 28001 to 28500 and identified at nucleotide positions 61 to 62, and wherein the sensitivity is at least 65% and specificity is at least 21%;
58. at least the CpGs defined by SEQ ID NOs 28501 to 29000 and identified at nucleotide positions 61 to 62, and wherein the sensitivity is at least 63% and specificity is at least 21%;
59. at least the CpGs defined by SEQ ID NOs 1 to 100 and identified at nucleotide positions 61 to 62, and wherein the sensitivity is at least 84% and specificity is at least 44%;
60. at least the CpGs defined by SEQ ID NOs 1 to 500 and identified at nucleotide positions 61 to 62, and wherein the sensitivity is at least 87% and specificity is at least 50%;
61. at least the CpGs defined by SEQ ID NOs 1 to 1000 and identified at nucleotide positions 61 to 62, and wherein the sensitivity is at least 89% and specificity is at least 49%;
62. at least the CpGs defined by SEQ ID NOs 1 to 2000 and identified at nucleotide positions 61 to 62, and wherein the sensitivity is at least 89% and specificity is at least 48%;
63. at least the CpGs defined by SEQ ID NOs 1 to 3000 and identified at nucleotide positions 61 to 62, and wherein the sensitivity is at least 92% and specificity is at least 49%;
64. at least the CpGs defined by SEQ ID NOs 1 to 4000 and identified at nucleotide positions 61 to 62, and wherein the sensitivity is at least 92% and specificity is at least 49%;
65. at least the CpGs defined by SEQ ID NOs 1 to 5000 and identified at nucleotide positions 61 to 62, and wherein the sensitivity is at least 93% and specificity is at least 50%;
66. at least the CpGs defined by SEQ ID NOs 1 to 6000 and identified at nucleotide positions 61 to 62, and wherein the sensitivity is at least 93% and specificity is at least 51%;
67. at least the CpGs defined by SEQ ID NOs 1 to 7000 and identified at nucleotide positions 61 to 62, and wherein the sensitivity is at least 93% and specificity is at least 50%;
68. at least the CpGs defined by SEQ ID NOs 1 to 8000 and identified at nucleotide positions 61 to 62, and wherein the sensitivity is at least 93% and specificity is at least 49%;
69. at least the CpGs defined by SEQ ID NOs 1 to 9000 and identified at nucleotide positions 61 to 62, and wherein the sensitivity is at least 93% and specificity is at least 50%;
70. at least the CpGs defined by SEQ ID NOs 1 to 10000 and identified at nucleotide positions 61 to 62, and wherein the sensitivity is at least 93% and specificity is at least 49%;
71. at least the CpGs defined by SEQ ID NOs 1 to 11000 and identified at nucleotide positions 61 to 62, and wherein the sensitivity is at least 93% and specificity is at least 50%;
72. at least the CpGs defined by SEQ ID NOs 1 to 12000 and identified at nucleotide positions 61 to 62, and wherein the sensitivity is at least 93% and specificity is at least 48%;
73. at least the CpGs defined by SEQ ID NOs 1 to 13000 and identified at nucleotide positions 61 to 62, and wherein the sensitivity is at least 94% and specificity is at least 48%;
74. at least the CpGs defined by SEQ ID NOs 1 to 14000 and identified at nucleotide positions 61 to 62, and wherein the sensitivity is at least 93% and specificity is at least 49%;
75. at least the CpGs defined by SEQ ID NOs 1 to 15000 and identified at nucleotide positions 61 to 62, and wherein the sensitivity is at least 94% and specificity is at least 48%;
76. at least the CpGs defined by SEQ ID NOs 1 to 16000 and identified at nucleotide positions 61 to 62, and wherein the sensitivity is at least 94% and specificity is at least 49%;
77. at least the CpGs defined by SEQ ID NOs 1 to 17000 and identified at nucleotide positions 61 to 62, and wherein the sensitivity is at least 94% and specificity is at least 48%;
78. at least the CpGs defined by SEQ ID NOs 1 to 18000 and identified at nucleotide positions 61 to 62, and wherein the sensitivity is at least 94% and specificity is at least 49%;
79. at least the CpGs defined by SEQ ID NOs 1 to 19000 and identified at nucleotide positions 61 to 62, and wherein the sensitivity is at least 94% and specificity is at least 49%;
80. at least the CpGs defined by SEQ ID NOs 1 to 20000 and identified at nucleotide positions 61 to 62, and wherein the sensitivity is at least 94% and specificity is at least 49%;
81. at least the CpGs defined by SEQ ID NOs 1 to 21000 and identified at nucleotide positions 61 to 62, and wherein the sensitivity is at least 94% and specificity is at least 49%;
82. at least the CpGs defined by SEQ ID NOs 1 to 22000 and identified at nucleotide positions 61 to 62, and wherein the sensitivity is at least 94% and specificity is at least 49%;
83. at least the CpGs defined by SEQ ID NOs 1 to 23000 and identified at nucleotide positions 61 to 62, and wherein the sensitivity is at least 94% and specificity is at least 49%;
84. at least the CpGs defined by SEQ ID NOs 1 to 24000 and identified at nucleotide positions 61 to 62, and wherein the sensitivity is at least 94% and specificity is at least 49%;
85. at least the CpGs defined by SEQ ID NOs 1 to 25000 and identified at nucleotide positions 61 to 62, and wherein the sensitivity is at least 94% and specificity is at least 49%;
86. at least the CpGs defined by SEQ ID NOs 1 to 26000 and identified at nucleotide positions 61 to 62, and wherein the sensitivity is at least 94% and specificity is at least 49%;
87. at least the CpGs defined by SEQ ID NOs 1 to 27000 and identified at nucleotide positions 61 to 62, and wherein the sensitivity is at least 94% and specificity is at least 49%;
88. at least the CpGs defined by SEQ ID NOs 1 to 28000 and identified at nucleotide positions 61 to 62, and wherein the sensitivity is at least 94% and specificity is at least 49%;
89. at least the CpGs defined by SEQ ID NOs 1 to 29000 and identified at nucleotide positions 61 to 62, and wherein the sensitivity is at least 94% and specificity is at least 50%;
90. at least the CpGs defined by SEQ ID NOs 28,001 to 29,000 and identified at nucleotide positions 61 to 62, and wherein the sensitivity is at least 62% and specificity is at least 54%;
91. at least the CpGs defined by SEQ ID NOs 27,001 to 29,000 and identified at nucleotide positions 61 to 62, and wherein the sensitivity is at least 64% and specificity is at least 52%;
92. at least the CpGs defined by SEQ ID NOs 26,001 to 29,000 and identified at nucleotide positions 61 to 62, and wherein the sensitivity is at least 62% and specificity is at least 52%;
93. at least the CpGs defined by SEQ ID NOs 25,001 to 29,000 and identified at nucleotide positions 61 to 62, and wherein the sensitivity is at least 62% and specificity is at least 51%;
94. at least the CpGs defined by SEQ ID NOs 24,001 to 29,000 and identified at nucleotide positions 61 to 62, and wherein the sensitivity is at least 62% and specificity is at least 52%;
95. at least the CpGs defined by SEQ ID NOs 23,001 to 29,000 and identified at nucleotide positions 61 to 62, and wherein the sensitivity is at least 64% and specificity is at least 51%;
96. at least the CpGs defined by SEQ ID NOs 22,001 to 29,000 and identified at nucleotide positions 61 to 62, and wherein the sensitivity is at least 63% and specificity is at least 51%;
97. at least the CpGs defined by SEQ ID NOs 21,001 to 29,000 and identified at nucleotide positions 61 to 62, and wherein the sensitivity is at least 63% and specificity is at least 51%;
98. at least the CpGs defined by SEQ ID NOs 20,001 to 29,000 and identified at nucleotide positions 61 to 62, and wherein the sensitivity is at least 64% and specificity is at least 52%;
99. at least the CpGs defined by SEQ ID NOs 19,001 to 29,000 and identified at nucleotide positions 61 to 62, and wherein the sensitivity is at least 62% and specificity is at least 53%;
100. at least the CpGs defined by SEQ ID NOs 18,001 to 29,000 and identified at nucleotide positions 61 to 62, and wherein the sensitivity is at least 62% and specificity is at least 53%;
101. at least the CpGs defined by SEQ ID NOs 17,001 to 29,000 and identified at nucleotide positions 61 to 62, and wherein the sensitivity is at least 63% and specificity is at least 53%;
102. at least the CpGs defined by SEQ ID NOs 16,001 to 29,000 and identified at nucleotide positions 61 to 62, and wherein the sensitivity is at least 64% and specificity is at least 54%;
103. at least the CpGs defined by SEQ ID NOs 15,001 to 29,000 and identified at nucleotide positions 61 to 62, and wherein the sensitivity is at least 63% and specificity is at least 53%;
104. at least the CpGs defined by SEQ ID NOs 14,001 to 29,000 and identified at nucleotide positions 61 to 62, and wherein the sensitivity is at least 63% and specificity is at least 53%;
105. at least the CpGs defined by SEQ ID NOs 13,001 to 29,000 and identified at nucleotide positions 61 to 62, and wherein the sensitivity is at least 63% and specificity is at least 53%;
106. at least the CpGs defined by SEQ ID NOs 12,001 to 29,000 and identified at nucleotide positions 61 to 62, and wherein the sensitivity is at least 63% and specificity is at least 53%;
107. at least the CpGs defined by SEQ ID NOs 11,001 to 29,000 and identified at nucleotide positions 61 to 62, and wherein the sensitivity is at least 63% and specificity is at least 53%;
108. at least the CpGs defined by SEQ ID NOs 10,001 to 29,000 and identified at nucleotide positions 61 to 62, and wherein the sensitivity is at least 65% and specificity is at least 52%;
109. at least the CpGs defined by SEQ ID NOs 9,001 to 29,000 and identified at nucleotide positions 61 to 62, and wherein the sensitivity is at least 64% and specificity is at least 52%;
110. at least the CpGs defined by SEQ ID NOs 8,001 to 29,000 and identified at nucleotide positions 61 to 62, and wherein the sensitivity is at least 71% and specificity is at least 53%;
111. at least the CpGs defined by SEQ ID NOs 7,001 to 29,000 and identified at nucleotide positions 61 to 62, and wherein the sensitivity is at least 65% and specificity is at least 52%;
112. at least the CpGs defined by SEQ ID NOs 6,001 to 29,000 and identified at nucleotide positions 61 to 62, and wherein the sensitivity is at least 71% and specificity is at least 53%;
113. at least the CpGs defined by SEQ ID NOs 5,001 to 29,000 and identified at nucleotide positions 61 to 62, and wherein the sensitivity is at least 78% and specificity is at least 53%;
114. at least the CpGs defined by SEQ ID NOs 4,001 to 29,000 and identified at nucleotide positions 61 to 62, and wherein the sensitivity is at least 79% and specificity is at least 53%;
115. at least the CpGs defined by SEQ ID NOs 3,001 to 29,000 and identified at nucleotide positions 61 to 62, and wherein the sensitivity is at least 83% and specificity is at least 53%;
116. at least the CpGs defined by SEQ ID NOs 2,001 to 29,000 and identified at nucleotide positions 61 to 62, and wherein the sensitivity is at least 86% and specificity is at least 53%;
117. at least the CpGs defined by SEQ ID NOs 1,001 to 29,000 and identified at nucleotide positions 61 to 62, and wherein the sensitivity is at least 89% and specificity is at least 52%;
118. at least the CpGs defined by SEQ ID NOs 501 to 29,000 and identified at nucleotide positions 61 to 62, and wherein the sensitivity is at least 93% and specificity is at least 52%;
119. at least the CpGs defined by SEQ ID NOs 101 to 29,000 and identified at nucleotide positions 61 to 62, and wherein the sensitivity is at least 94% and specificity is at least 51%; or
120. at least the CpGs defined by SEQ ID NOs 1 to 29,000 and identified at nucleotide positions 61 to 62, and wherein the sensitivity is at least 94% and specificity is at least 50%.

In any of the described assays, the methylation status of the one or more CpGs in the panel is preferably determined by a β-value analysis, and the cancer is breast cancer or ovarian cancer. Preferably, the cancer is breast cancer.

In any of the assays described herein, wherein when the cancer index value for the individual is about 0.126 or more, the individual is assessed as having cancer or as having a high risk of cancer development, or wherein when the cancer index value for the individual is less than about 0.126, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-BC-Index. The panel of one or more CpGs used to derive the cancer index value may particularly comprise:
a. at least 500 of the CpGs identified at nucleotide positions 61 to 62 in SEQ ID NOs 1 to 29,000, and wherein the sensitivity is at least 48% and the specificity is at least 39%;
b. at least the CpGs defined by SEQ ID NOs 1 to 500 and identified at nucleotide positions 61 to 62, and wherein the sensitivity is at least 70% and specificity is at least 72%;
c. at least the CpGs defined by SEQ ID NOs 1 to 1000 and identified at nucleotide positions 61 to 62, and wherein the sensitivity is at least 70% and specificity is at least 78%;
d. at least the CpGs defined by SEQ ID NOs 1 to 2000 and identified at nucleotide positions 61 to 62, and wherein the sensitivity is at least 74% and specificity is at least 78%.

In any of the assays described herein, wherein when the cancer index value for the individual is about 0.126 or more, the individual is assessed as having cancer or as having a high risk of cancer development, or wherein when the cancer index value for the individual is less than about 0.126, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-BC-Index. The panel of one or more CpGs used to derive the cancer index value may particularly comprise:
1. at least the CpGs defined by SEQ ID NOs 1 to 500 and identified at nucleotide positions 61 to 62, and wherein the sensitivity is at least 70% and specificity is at least 72%;
2. at least the CpGs defined by SEQ ID NOs 501 to 100 and identified at nucleotide positions 61 to 62, and wherein the sensitivity is at least 71% and specificity is at least 74%; or
3. at least the CpGs defined by SEQ ID NOs 1001 to 1500 and identified at nucleotide positions 61 to 62, and wherein the sensitivity is at least 67% and specificity is at least 74%;
4. at least the CpGs defined by SEQ ID NOs 1501 to 2000 and identified at nucleotide positions 61 to 62, and wherein the sensitivity is at least 68% and specificity is at least 68%;
5. at least the CpGs defined by SEQ ID NOs 2001 to 2500 and identified at nucleotide positions 61 to 62, and wherein the sensitivity is at least 71% and specificity is at least 72%;
6. at least the CpGs defined by SEQ ID NOs 2501 to 3000 and identified at nucleotide positions 61 to 62, and wherein the sensitivity is at least 61% and specificity is at least 71%;
7. at least the CpGs defined by SEQ ID NOs 3001 to 3500 and identified at nucleotide positions 61 to 62, and wherein the sensitivity is at least 64% and specificity is at least 75%;
8. at least the CpGs defined by SEQ ID NOs 3501 to 4000 and identified at nucleotide positions 61 to 62, and wherein the sensitivity is at least 56% and specificity is at least 70%;
9. at least the CpGs defined by SEQ ID NOs 4001 to 4500 and identified at nucleotide positions 61 to 62, and wherein the sensitivity is at least 61% and specificity is at least 69%;
10. at least the CpGs defined by SEQ ID NOs 4501 to 5000 and identified at nucleotide positions 61 to 62, and wherein the sensitivity is at least 66% and specificity is at least 72%;
11. at least the CpGs defined by SEQ ID NOs 5001 to 5500 and identified at nucleotide positions 61 to 62, and wherein the sensitivity is at least 74% and specificity is at least 69%;
12. at least the CpGs defined by SEQ ID NOs 5501 to 6000 and identified at nucleotide positions 61 to 62, and wherein the sensitivity is at least 62% and specificity is at least 71%;
13. at least the CpGs defined by SEQ ID NOs 6001 to 6500 and identified at nucleotide positions 61 to 62, and wherein the sensitivity is at least 57% and specificity is at least 69%;
14. at least the CpGs defined by SEQ ID NOs 6501 to 7000 and identified at nucleotide positions 61 to 62, and wherein the sensitivity is at least 65% and specificity is at least 69%;
15. at least the CpGs defined by SEQ ID NOs 7001 to 7500 and identified at nucleotide positions 61 to 62, and wherein the sensitivity is at least 55% and specificity is at least 69%;
16. at least the CpGs defined by SEQ ID NOs 7501 to 8000 and identified at nucleotide positions 61 to 62, and wherein the sensitivity is at least 59% and specificity is at least 68%;
17. at least the CpGs defined by SEQ ID NOs 8001 to 8500 and identified at nucleotide positions 61 to 62, and wherein the sensitivity is at least 62% and specificity is at least 70%;
18. at least the CpGs defined by SEQ ID NOs 8501 to 9000 and identified at nucleotide positions 61 to 62, and wherein the sensitivity is at least 59% and specificity is at least 73%;
19. at least the CpGs defined by SEQ ID NOs 9001 to 9500 and identified at nucleotide positions 61 to 62, and wherein the sensitivity is at least 59% and specificity is at least 71%;
20. at least the CpGs defined by SEQ ID NOs 9501 to 10000 and identified at nucleotide positions 61 to 62, and wherein the sensitivity is at least 54% and specificity is at least 71%;
21. at least the CpGs defined by SEQ ID NOs 10001 to 10500 and identified at nucleotide positions 61 to 62, and wherein the sensitivity is at least 52% and specificity is at least 72%;
22. at least the CpGs defined by SEQ ID NOs 10501 to 11000 and identified at nucleotide positions 61 to 62, and wherein the sensitivity is at least 54% and specificity is at least 72%;
23. at least the CpGs defined by SEQ ID NOs 11001 to 11500 and identified at nucleotide positions 61 to 62, and wherein the sensitivity is at least 55% and specificity is at least 69%;
24. at least the CpGs defined by SEQ ID NOs 11501 to 12000 and identified at nucleotide positions 61 to 62, and wherein the sensitivity is at least 48% and specificity is at least 70%;
25. at least the CpGs defined by SEQ ID NOs 12001 to 12500 and identified at nucleotide positions 61 to 62, and wherein the sensitivity is at least 54% and specificity is at least 68%;
26. at least the CpGs defined by SEQ ID NOs 12501 to 13000 and identified at nucleotide positions 61 to 62, and wherein the sensitivity is at least 51% and specificity is at least 72%;
27. at least the CpGs defined by SEQ ID NOs 13001 to 13500 and identified at nucleotide positions 61 to 62, and wherein the sensitivity is at least 55% and specificity is at least 72%;
28. at least the CpGs defined by SEQ ID NOs 13501 to 14000 and identified at nucleotide positions 61 to 62, and wherein the sensitivity is at least 50% and specificity is at least 73%;
29. at least the CpGs defined by SEQ ID NOs 14001 to 14500 and identified at nucleotide positions 61 to 62, and wherein the sensitivity is at least 48% and specificity is at least 73%;
30. at least the CpGs defined by SEQ ID NOs 14501 to 15000 and identified at nucleotide positions 61 to 62, and wherein the sensitivity is at least 38% and specificity is at least 73%;
31. at least the CpGs defined by SEQ ID NOs 15001 to 15500 and identified at nucleotide positions 61 to 62, and wherein the sensitivity is at least 54% and specificity is at least 71%;
32. at least the CpGs defined by SEQ ID NOs 15501 to 16000 and identified at nucleotide positions 61 to 62, and wherein the sensitivity is at least 46% and specificity is at least 39%;
33. at least the CpGs defined by SEQ ID NOs 16001 to 16500 and identified at nucleotide positions 61 to 62, and wherein the sensitivity is at least 44% and specificity is at least 40%;
34. at least the CpGs defined by SEQ ID NOs 16501 to 17000 and identified at nucleotide positions 61 to 62, and wherein the sensitivity is at least 38% and specificity is at least 40%;
35. at least the CpGs defined by SEQ ID NOs 17001 to 17500 and identified at nucleotide positions 61 to 62, and wherein the sensitivity is at least 46% and specificity is at least 38%;
36. at least the CpGs defined by SEQ ID NOs 17501 to 18000 and identified at nucleotide positions 61 to 62, and wherein the sensitivity is at least 42% and specificity is at least 38%;
37. at least the CpGs defined by SEQ ID NOs 18001 to 18500 and identified at nucleotide positions 61 to 62, and wherein the sensitivity is at least 44% and specificity is at least 39%;
38. at least the CpGs defined by SEQ ID NOs 18501 to 19000 and identified at nucleotide positions 61 to 62, and wherein the sensitivity is at least 45% and specificity is at least 41%;
39. at least the CpGs defined by SEQ ID NOs 19001 to 19500 and identified at nucleotide positions 61 to 62, and wherein the sensitivity is at least 48% and specificity is at least 38%;
40. at least the CpGs defined by SEQ ID NOs 19501 to 20000 and identified at nucleotide positions 61 to 62, and wherein the sensitivity is at least 46% and specificity is at least 41%;
41. at least the CpGs defined by SEQ ID NOs 20001 to 20500 and identified at nucleotide positions 61 to 62, and wherein the sensitivity is at least 41% and specificity is at least 40%;
42. at least the CpGs defined by SEQ ID NOs 20501 to 21000 and identified at nucleotide positions 61 to 62, and wherein the sensitivity is at least 46% and specificity is at least 42%;
43. at least the CpGs defined by SEQ ID NOs 21001 to 21500 and identified at nucleotide positions 61 to 62, and wherein the sensitivity is at least 46% and specificity is at least 39%;
44. at least the CpGs defined by SEQ ID NOs 21501 to 22000 and identified at nucleotide positions 61 to 62, and wherein the sensitivity is at least 43% and specificity is at least 39%;
45. at least the CpGs defined by SEQ ID NOs 22001 to 22500 and identified at nucleotide positions 61 to 62, and wherein the sensitivity is at least 38% and specificity is at least 41%;
46. at least the CpGs defined by SEQ ID NOs 22501 to 23000 and identified at nucleotide positions 61 to 62, and wherein the sensitivity is at least 45% and specificity is at least 39%;
47. at least the CpGs defined by SEQ ID NOs 23001 to 23500 and identified at nucleotide positions 61 to 62, and wherein the sensitivity is at least 47% and specificity is at least 40%;
48. at least the CpGs defined by SEQ ID NOs 23501 to 24000 and identified at nucleotide positions 61 to 62, and wherein the sensitivity is at least 50% and specificity is at least 37%;
49. at least the CpGs defined by SEQ ID NOs 24001 to 24500 and identified at nucleotide positions 61 to 62, and wherein the sensitivity is at least 42% and specificity is at least 39%;
50. at least the CpGs defined by SEQ ID NOs 24501 to 25000 and identified at nucleotide positions 61 to 62, and wherein the sensitivity is at least 50% and specificity is at least 37%;
51. at least the CpGs defined by SEQ ID NOs 25001 to 25500 and identified at nucleotide positions 61 to 62, and wherein the sensitivity is at least 46% and specificity is at least 40%;
52. at least the CpGs defined by SEQ ID NOs 25501 to 26000 and identified at nucleotide positions 61 to 62, and wherein the sensitivity is at least 45% and specificity is at least 44%;
53. at least the CpGs defined by SEQ ID NOs 26001 to 26500 and identified at nucleotide positions 61 to 62, and wherein the sensitivity is at least 50% and specificity is at least 41%;
54. at least the CpGs defined by SEQ ID NOs 26501 to 27000 and identified at nucleotide positions 61 to 62, and wherein the sensitivity is at least 44% and specificity is at least 39%;
55. at least the CpGs defined by SEQ ID NOs 27001 to 27500 and identified at nucleotide positions 61 to 62, and wherein the sensitivity is at least 48% and specificity is at least 45%;
56. at least the CpGs defined by SEQ ID NOs 27501 to 28000 and identified at nucleotide positions 61 to 62, and wherein the sensitivity is at least 44% and specificity is at least 40%;
57. at least the CpGs defined by SEQ ID NOs 28001 to 28500 and identified at nucleotide positions 61 to 62, and wherein the sensitivity is at least 48% and specificity is at least 40%;
58. at least the CpGs defined by SEQ ID NOs 28501 to 29000 and identified at nucleotide positions 61 to 62, and wherein the sensitivity is at least 48% and specificity is at least 39%;
59. least the CpGs defined by SEQ ID NOs 1 to 100 and identified at nucleotide positions 61 to 62, and wherein the sensitivity is at least 66% and specificity is at least 64%;
60. at least the CpGs defined by SEQ ID NOs 1 to 500 and identified at nucleotide positions 61 to 62, and wherein the sensitivity is at least 70% and specificity is at least 72%;
61. at least the CpGs defined by SEQ ID NOs 1 to 1000 and identified at nucleotide positions 61 to 62, and wherein the sensitivity is at least 70% and specificity is at least 78%;
62. at least the CpGs defined by SEQ ID NOs 1 to 2000 and identified at nucleotide positions 61 to 62, and wherein the sensitivity is at least 74% and specificity is at least 78%;
63. at least the CpGs defined by SEQ ID NOs 1 to 3000 and identified at nucleotide positions 61 to 62, and wherein the sensitivity is at least 75% and specificity is at least 77%;
64. at least the CpGs defined by SEQ ID NOs 1 to 4000 and identified at nucleotide positions 61 to 62, and wherein the sensitivity is at least 72% and specificity is at least 77%;
65. at least the CpGs defined by SEQ ID NOs 1 to 5000 and identified at nucleotide positions 61 to 62, and wherein the sensitivity is at least 73% and specificity is at least 79%;
66. at least the CpGs defined by SEQ ID NOs 1 to 6000 and identified at nucleotide positions 61 to 62, and wherein the sensitivity is at least 72% and specificity is at least 79%;
67. at least the CpGs defined by SEQ ID NOs 1 to 7000 and identified at nucleotide positions 61 to 62, and wherein the sensitivity is at least 72% and specificity is at least 78%;
68. at least the CpGs defined by SEQ ID NOs 1 to 8000 and identified at nucleotide positions 61 to 62, and wherein the sensitivity is at least 72% and specificity is at least 79%;
69. at least the CpGs defined by SEQ ID NOs 1 to 9000 and identified at nucleotide positions 61 to 62, and wherein the sensitivity is at least 72% and specificity is at least 78%;
70. at least the CpGs defined by SEQ ID NOs 1 to 10000 and identified at nucleotide positions 61 to 62, and wherein the sensitivity is at least 73% and specificity is at least 79%;
71. at least the CpGs defined by SEQ ID NOs 1 to 11000 and identified at nucleotide positions 61 to 62, and wherein the sensitivity is at least 73% and specificity is at least 78%;
72. at least the CpGs defined by SEQ ID NOs 1 to 12000 and identified at nucleotide positions 61 to 62, and wherein the sensitivity is at least 73% and specificity is at least 78%;
73. at least the CpGs defined by SEQ ID NOs 1 to 13000 and identified at nucleotide positions 61 to 62, and wherein the sensitivity is at least 73% and specificity is at least 79%;
74. at least the CpGs defined by SEQ ID NOs 1 to 14000 and identified at nucleotide positions 61 to 62, and wherein the sensitivity is at least 73% and specificity is at least 79%;
75. at least the CpGs defined by SEQ ID NOs 1 to 15000 and identified at nucleotide positions 61 to 62, and wherein the sensitivity is at least 73% and specificity is at least 79%;
76. at least the CpGs defined by SEQ ID NOs 1 to 16000 and identified at nucleotide positions 61 to 62, and wherein the sensitivity is at least 73% and specificity is at least 80%;
77. at least the CpGs defined by SEQ ID NOs 1 to 17000 and identified at nucleotide positions 61 to 62, and wherein the sensitivity is at least 73% and specificity is at least 80%;
78. at least the CpGs defined by SEQ ID NOs 1 to 18000 and identified at nucleotide positions 61 to 62, and wherein the sensitivity is at least 73% and specificity is at least 80%;
79. at least the CpGs defined by SEQ ID NOs 1 to 19000 and identified at nucleotide positions 61 to 62, and wherein the sensitivity is at least 73% and specificity is at least 80%;
80. at least the CpGs defined by SEQ ID NOs 1 to 20000 and identified at nucleotide positions 61 to 62, and wherein the sensitivity is at least 73% and specificity is at least 79%;
81. at least the CpGs defined by SEQ ID NOs 1 to 21000 and identified at nucleotide positions 61 to 62, and wherein the sensitivity is at least 73% and specificity is at least 79%;
82. at least the CpGs defined by SEQ ID NOs 1 to 22000 and identified at nucleotide positions 61 to 62, and wherein the sensitivity is at least 73% and specificity is at least 79%;
83. at least the CpGs defined by SEQ ID NOs 1 to 23000 and identified at nucleotide positions 61 to 62, and wherein the sensitivity is at least 73% and specificity is at least 79%;
84. at least the CpGs defined by SEQ ID NOs 1 to 24000 and identified at nucleotide positions 61 to 62, and wherein the sensitivity is at least 73% and specificity is at least 80%;
85. at least the CpGs defined by SEQ ID NOs 1 to 25000 and identified at nucleotide positions 61 to 62, and wherein the sensitivity is at least 73% and specificity is at least 80%;
86. at least the CpGs defined by SEQ ID NOs 1 to 26000 and identified at nucleotide positions 61 to 62, and wherein the sensitivity is at least 73% and specificity is at least 80%;
87. at least the CpGs defined by SEQ ID NOs 1 to 27000 and identified at nucleotide positions 61 to 62, and wherein the sensitivity is at least 73% and specificity is at least 80%;
88. at least the CpGs defined by SEQ ID NOs 1 to 28000 and identified at nucleotide positions 61 to 62, and wherein the sensitivity is at least 73% and specificity is at least 80%;
89. at least the CpGs defined by SEQ ID NOs 1 to 29000 and identified at nucleotide positions 61 to 62, and wherein the sensitivity is at least 73% and specificity is at least 80%;
90. at least the CpGs defined by SEQ ID NOs 28,001 to 29,000 and identified at nucleotide positions 61 to 62, and wherein the sensitivity is at least 41% and specificity is at least 74%;
91. at least the CpGs defined by SEQ ID NOs 27,001 to 29,000 and identified at nucleotide positions 61 to 62, and wherein the sensitivity is at least 42% and specificity is at least 73%;
92. at least the CpGs defined by SEQ ID NOs 26,001 to 29,000 and identified at nucleotide positions 61 to 62, and wherein the sensitivity is at least 44% and specificity is at least 71%;
93. at least the CpGs defined by SEQ ID NOs 25,001 to 29,000 and identified at nucleotide positions 61 to 62, and wherein the sensitivity is at least 42% and specificity is at least 72%;
94. at least the CpGs defined by SEQ ID NOs 24,001 to 29,000 and identified at nucleotide positions 61 to 62, and wherein the sensitivity is at least 40% and specificity is at least 72%;
95. at least the CpGs defined by SEQ ID NOs 23,001 to 29,000 and identified at nucleotide positions 61 to 62, and wherein the sensitivity is at least 43% and specificity is at least 73%;
96. at least the CpGs defined by SEQ ID NOs 22,001 to 29,000 and identified at nucleotide positions 61 to 62, and wherein the sensitivity is at least 43% and specificity is at least 73%;
97. at least the CpGs defined by SEQ ID NOs 21,001 to 29,000 and identified at nucleotide positions 61 to 62, and wherein the sensitivity is at least 41% and specificity is at least 72%;
98. at least the CpGs defined by SEQ ID NOs 20,001 to 29,000 and identified at nucleotide positions 61 to 62, and wherein the sensitivity is at least 45% and specificity is at least 72%;
99. at least the CpGs defined by SEQ ID NOs 19,001 to 29,000 and identified at nucleotide positions 61 to 62, and wherein the sensitivity is at least 43% and specificity is at least 72%;
100. at least the CpGs defined by SEQ ID NOs 18,001 to 29,000 and identified at nucleotide positions 61 to 62, and wherein the sensitivity is at least 42% and specificity is at least 72%;
101. at least the CpGs defined by SEQ ID NOs 17,001 to 29,000 and identified at nucleotide positions 61 to 62, and wherein the sensitivity is at least 43% and specificity is at least 72%;
102. at least the CpGs defined by SEQ ID NOs 16,001 to 29,000 and identified at nucleotide positions 61 to 62, and wherein the sensitivity is at least 45% and specificity is at least 72%;
103. at least the CpGs defined by SEQ ID NOs 15,001 to 29,000 and identified at nucleotide positions 61 to 62, and wherein the sensitivity is at least 45% and specificity is at least 72%;
104. at least the CpGs defined by SEQ ID NOs 14,001 to 29,000 and identified at nucleotide positions 61 to 62, and wherein the sensitivity is at least 45% and specificity is at least 72%;
105. at least the CpGs defined by SEQ ID NOs 13,001 to 29,000 and identified at nucleotide positions 61 to 62, and wherein the sensitivity is at least 46% and specificity is at least 72%;
106. at least the CpGs defined by SEQ ID NOs 12,001 to 29,000 and identified at nucleotide positions 61 to 62, and wherein the sensitivity is at least 46% and specificity is at least 72%;
107. at least the CpGs defined by SEQ ID NOs 11,001 to 29,000 and identified at nucleotide positions 61 to 62, and wherein the sensitivity is at least 46% and specificity is at least 72%;
108. at least the CpGs defined by SEQ ID NOs 10,001 to 29,000 and identified at nucleotide positions 61 to 62, and wherein the sensitivity is at least 50% and specificity is at least 71%;
109. at least the CpGs defined by SEQ ID NOs 9,001 to 29,000 and identified at nucleotide positions 61 to 62, and wherein the sensitivity is at least 48% and specificity is at least 72%;
110. at least the CpGs defined by SEQ ID NOs 8,001 to 29,000 and identified at nucleotide positions 61 to 62, and wherein the sensitivity is at least 54% and specificity is at least 71%;
111. at least the CpGs defined by SEQ ID NOs 7,001 to 29,000 and identified at nucleotide positions 61 to 62, and wherein the sensitivity is at least 50% and specificity is at least 72%;
112. at least the CpGs defined by SEQ ID NOs 6,001 to 29,000 and identified at nucleotide positions 61 to 62, and wherein the sensitivity is at least 53% and specificity is at least 72%;
113. at least the CpGs defined by SEQ ID NOs 5,001 to 29,000 and identified at nucleotide positions 61 to 62, and wherein the sensitivity is at least 54% and specificity is at least 72%;
114. at least the CpGs defined by SEQ ID NOs 4,001 to 29,000 and identified at nucleotide positions 61 to 62, and wherein the sensitivity is at least 56% and specificity is at least 72%;
115. at least the CpGs defined by SEQ ID NOs 3,001 to 29,000 and identified at nucleotide positions 61 to 62, and wherein the sensitivity is at least 59% and specificity is at least 72%;
116. at least the CpGs defined by SEQ ID NOs 2,001 to 29,000 and identified at nucleotide positions 61 to 62, and wherein the sensitivity is at least 61% and specificity is at least 73%;
117. at least the CpGs defined by SEQ ID NOs 1,001 to 29,000 and identified at nucleotide positions 61 to 62, and wherein the sensitivity is at least 69% and specificity is at least 76%;
118. at least the CpGs defined by SEQ ID NOs 501 to 29,000 and identified at nucleotide positions 61 to 62, and wherein the sensitivity is at least 75% and specificity is at least 77%;
119. at least the CpGs defined by SEQ ID NOs 101 to 29,000 and identified at nucleotide positions 61 to 62, and wherein the sensitivity is at least 76% and specificity is at least 81%; or
120. at least the CpGs defined by SEQ ID NOs 1 to 29,000 and identified at nucleotide positions 61 to 62, and wherein the sensitivity is at least 73% and specificity is at least 80%.

In any of the described assays, the methylation status of the one or more CpGs in the panel is preferably determined by a β-value analysis, and the cancer is breast cancer or ovarian cancer. Preferably, the cancer is breast cancer.

In any of the assays described herein, wherein when the cancer index value for the individual is about 0.743 or more, the individual is assessed as having cancer or as having a high risk of cancer development, or wherein when the cancer index value for the individual is less than about 0.743, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-BC-Index. The panel of one or more CpGs used to derive the cancer index value may particularly comprise:
a. at least 500 of the CpGs identified at nucleotide positions 61 to 62 in SEQ ID NOs 1 to 29,000, and wherein the sensitivity is at least 17% and the specificity is at least 77%;
b. at least the CpGs defined by SEQ ID NOs 1 to 500 and identified at nucleotide positions 61 to 62, and wherein the sensitivity is at least 30% and specificity is at least 95%;
c. at least the CpGs defined by SEQ ID NOs 1 to 1000 and identified at nucleotide positions 61 to 62, and wherein the sensitivity is at least 31% and specificity is at least 96%;
d. at least the CpGs defined by SEQ ID NOs 1 to 2000 and identified at nucleotide positions 61 to 62, and wherein the sensitivity is at least 29% and specificity is at least 96%.

In any of the assays described herein, wherein when the cancer index value for the individual is about 0.743 or more, the individual is assessed as having cancer or as having a high risk of cancer development, or wherein when the cancer index value for the individual is less than about 0.743, the individual is assessed as not having cancer or as having a low risk of cancer development, preferably wherein the assay comprises determining methylation β-values for each CpG in the panel of one or more CpGs, and more preferably wherein the assessing the presence, absence or development of cancer in an individual is based on the WID-BC-Index. The panel of one or more CpGs used to derive the cancer index value may particularly comprise:
1. at least the CpGs defined by SEQ ID NOs 1 to 500 and identified at nucleotide positions 61 to 62, and wherein the sensitivity is at least 30% and specificity is at least 95%;
2. at least the CpGs defined by SEQ ID NOs 501 to 1000 and identified at nucleotide positions 61 to 62, and wherein the sensitivity is at least 30% and specificity is at least 93%; or
3. at least the CpGs defined by SEQ ID NOs 1001 to 1500 and identified at nucleotide positions 61 to 62, and wherein the sensitivity is at least 31% and specificity is at least 91%;
4. at least the CpGs defined by SEQ ID NOs 1501 to 2000 and identified at nucleotide positions 61 to 62, and wherein the sensitivity is at least 29% and specificity is at least 92%;
5. at least the CpGs defined by SEQ ID NOs 2001 to 2500 and identified at nucleotide positions 61 to 62, and wherein the sensitivity is at least 35% and specificity is at least 90%;
6. at least the CpGs defined by SEQ ID NOs 2501 to 3000 and identified at nucleotide positions 61 to 62, and wherein the sensitivity is at least 33% and specificity is at least 89%;
7. at least the CpGs defined by SEQ ID NOs 3001 to 3500 and identified at nucleotide positions 61 to 62, and wherein the sensitivity is at least 34% and specificity is at least 93%;
8. at least the CpGs defined by SEQ ID NOs 3501 to 4000 and identified at nucleotide positions 61 to 62, and wherein the sensitivity is at least 29% and specificity is at least 91%;
9. at least the CpGs defined by SEQ ID NOs 4001 to 4500 and identified at nucleotide positions 61 to 62, and wherein the sensitivity is at least 33% and specificity is at least 90%;
10. at least the CpGs defined by SEQ ID NOs 4501 to 5000 and identified at nucleotide positions 61 to 62, and wherein the sensitivity is at least 36% and specificity is at least 92%;
11. at least the CpGs defined by SEQ ID NOs 5001 to 5500 and identified at nucleotide positions 61 to 62, and wherein the sensitivity is at least 30% and specificity is at least 88%;
12. at least the CpGs defined by SEQ ID NOs 5501 to 6000 and identified at nucleotide positions 61 to 62, and wherein the sensitivity is at least 35% and specificity is at least 90%;
13. at least the CpGs defined by SEQ ID NOs 6001 to 6500 and identified at nucleotide positions 61 to 62, and wherein the sensitivity is at least 28% and specificity is at least 92%;
14. at least the CpGs defined by SEQ ID NOs 6501 to 7000 and identified at nucleotide positions 61 to 62, and wherein the sensitivity is at least 30% and specificity is at least 92%;
15. at least the CpGs defined by SEQ ID NOs 7001 to 7500 and identified at nucleotide positions 61 to 62, and wherein the sensitivity is at least 29% and specificity is at least 89%;
16. at least the CpGs defined by SEQ ID NOs 7501 to 8000 and identified at nucleotide positions 61 to 62, and wherein the sensitivity is at least 31% and specificity is at least 87%;
17. at least the CpGs defined by SEQ ID NOs 8001 to 8500 and identified at nucleotide positions 61 to 62, and wherein the sensitivity is at least 34% and specificity is at least 86%;
18. at least the CpGs defined by SEQ ID NOs 8501 to 9000 and identified at nucleotide positions 61 to 62, and wherein the sensitivity is at least 29% and specificity is at least 91%;
19. at least the CpGs defined by SEQ ID NOs 9001 to 9500 and identified at nucleotide positions 61 to 62, and wherein the sensitivity is at least 28% and specificity is at least 89%;
20. at least the CpGs defined by SEQ ID NOs 9501 to 10000 and identified at nucleotide positions 61 to 62, and wherein the sensitivity is at least 34% and specificity is at least 87%;
21. at least the CpGs defined by SEQ ID NOs 10001 to 10500 and identified at nucleotide positions 61 to 62, and wherein the sensitivity is at least 27% and specificity is at least 87%;
22. at least the CpGs defined by SEQ ID NOs 10501 to 11000 and identified at nucleotide positions 61 to 62, and wherein the sensitivity is at least 28% and specificity is at least 88%;
23. at least the CpGs defined by SEQ ID NOs 11001 to 11500 and identified at nucleotide positions 61 to 62, and wherein the sensitivity is at least 30% and specificity is at least 87%;
24. at least the CpGs defined by SEQ ID NOs 11501 to 12000 and identified at nucleotide positions 61 to 62, and wherein the sensitivity is at least 24% and specificity is at least 88%;
25. at least the CpGs defined by SEQ ID NOs 12001 to 12500 and identified at nucleotide positions 61 to 62, and wherein the sensitivity is at least 28% and specificity is at least 89%;
26. at least the CpGs defined by SEQ ID NOs 12501 to 13000 and identified at nucleotide positions 61 to 62, and wherein the sensitivity is at least 30% and specificity is at least 89%;
27. at least the CpGs defined by SEQ ID NOs 13001 to 13500 and identified at nucleotide positions 61 to 62, and wherein the sensitivity is at least 29% and specificity is at least 89%;
28. at least the CpGs defined by SEQ ID NOs 13501 to 14000 and identified at nucleotide positions 61 to 62, and wherein the sensitivity is at least 26% and specificity is at least 89%;
29. at least the CpGs defined by SEQ ID NOs 14001 to 14500 and identified at nucleotide positions 61 to 62, and wherein the sensitivity is at least 26% and specificity is at least 88%;
30. at least the CpGs defined by SEQ ID NOs 14501 to 15000 and identified at nucleotide positions 61 to 62, and wherein the sensitivity is at least 24% and specificity is at least 88%;
31. at least the CpGs defined by SEQ ID NOs 15001 to 15500 and identified at nucleotide positions 61 to 62, and wherein the sensitivity is at least 24% and specificity is at least 89%;
32. at least the CpGs defined by SEQ ID NOs 15501 to 16000 and identified at nucleotide positions 61 to 62, and wherein the sensitivity is at least 11% and specificity is at least 72%;
33. at least the CpGs defined by SEQ ID NOs 16001 to 16500 and identified at nucleotide positions 61 to 62, and wherein the sensitivity is at least 13% and specificity is at least 73%;
34. at least the CpGs defined by SEQ ID NOs 16501 to 17000 and identified at nucleotide positions 61 to 62, and wherein the sensitivity is at least 12% and specificity is at least 70%;
35. at least the CpGs defined by SEQ ID NOs 17001 to 17500 and identified at nucleotide positions 61 to 62, and wherein the sensitivity is at least 15% and specificity is at least 74%;
36. at least the CpGs defined by SEQ ID NOs 17501 to 18000 and identified at nucleotide positions 61 to 62, and wherein the sensitivity is at least 15% and specificity is at least 75%;
37. at least the CpGs defined by SEQ ID NOs 18001 to 18500 and identified at nucleotide positions 61 to 62, and wherein the sensitivity is at least 15% and specificity is at least 75%;
38. at least the CpGs defined by SEQ ID NOs 18501 to 19000 and identified at nucleotide positions 61 to 62, and wherein the sensitivity is at least 17% and specificity is at least 73%;
39. at least the CpGs defined by SEQ ID NOs 19001 to 19500 and identified at nucleotide positions 61 to 62, and wherein the sensitivity is at least 12% and specificity is at least 74%;
40. at least the CpGs defined by SEQ ID NOs 19501 to 20000 and identified at nucleotide positions 61 to 62, and wherein the sensitivity is at least 16% and specificity is at least 70%;
41. at least the CpGs defined by SEQ ID NOs 20001 to 20500 and identified at nucleotide positions 61 to 62, and wherein the sensitivity is at least 12% and specificity is at least 74%;
42. at least the CpGs defined by SEQ ID NOs 20501 to 21000 and identified at nucleotide positions 61 to 62, and wherein the sensitivity is at least 15% and specificity is at least 72%;
43. at least the CpGs defined by SEQ ID NOs 21001 to 21500 and identified at nucleotide positions 61 to 62, and wherein the sensitivity is at least 15% and specificity is at least 72%;
44. at least the CpGs defined by SEQ ID NOs 21501 to 22000 and identified at nucleotide positions 61 to 62, and wherein the sensitivity is at least 12% and specificity is at least 74%;
45. at least the CpGs defined by SEQ ID NOs 22001 to 22500 and identified at nucleotide positions 61 to 62, and wherein the sensitivity is at least 12% and specificity is at least 72%;
46. at least the CpGs defined by SEQ ID NOs 22501 to 23000 and identified at nucleotide positions 61 to 62, and wherein the sensitivity is at least 17% and specificity is at least 74%;
47. at least the CpGs defined by SEQ ID NOs 23001 to 23500 and identified at nucleotide positions 61 to 62, and wherein the sensitivity is at least 21% and specificity is at least 76%;
48. at least the CpGs defined by SEQ ID NOs 23501 to 24000 and identified at nucleotide positions 61 to 62, and wherein the sensitivity is at least 18% and specificity is at least 73%;
49. at least the CpGs defined by SEQ ID NOs 24001 to 24500 and identified at nucleotide positions 61 to 62, and wherein the sensitivity is at least 13% and specificity is at least 72%;
50. at least the CpGs defined by SEQ ID NOs 24501 to 25000 and identified at nucleotide positions 61 to 62, and wherein the sensitivity is at least 16% and specificity is at least 72%;
51. at least the CpGs defined by SEQ ID NOs 25001 to 25500 and identified at nucleotide positions 61 to 62, and wherein the sensitivity is at least 19% and specificity is at least 75%;
52. at least the CpGs defined by SEQ ID NOs 25501 to 26000 and identified at nucleotide positions 61 to 62, and wherein the sensitivity is at least 19% and specificity is at least 75%;
53. at least the CpGs defined by SEQ ID NOs 26001 to 26500 and identified at nucleotide positions 61 to 62, and wherein the sensitivity is at least 16% and specificity is at least 75%;
54. at least the CpGs defined by SEQ ID NOs 26501 to 27000 and identified at nucleotide positions 61 to 62, and wherein the sensitivity is at least 20% and specificity is at least 72%;
55. at least the CpGs defined by SEQ ID NOs 27001 to 27500 and identified at nucleotide positions 61 to 62, and wherein the sensitivity is at least 24% and specificity is at least 77%;
56. at least the CpGs defined by SEQ ID NOs 27501 to 28000 and identified at nucleotide positions 61 to 62, and wherein the sensitivity is at least 16% and specificity is at least 75%;
57. at least the CpGs defined by SEQ ID NOs 28001 to 28500 and identified at nucleotide positions 61 to 62, and wherein the sensitivity is at least 21% and specificity is at least 73%;
58. at least the CpGs defined by SEQ ID NOs 28501 to 29000 and identified at nucleotide positions 61 to 62, and wherein the sensitivity is at least 17% and specificity is at least 77%;
59. at least the CpGs defined by SEQ ID NOs 1 to 100 and identified at nucleotide positions 61 to 62, and wherein the sensitivity is at least 35% and specificity is at least 88%;
60. at least the CpGs defined by SEQ ID NOs 1 to 500 and identified at nucleotide positions 61 to 62, and wherein the sensitivity is at least 30% and specificity is at least 95%;
61. at least the CpGs defined by SEQ ID NOs 1 to 1000 and identified at nucleotide positions 61 to 62, and wherein the sensitivity is at least 31% and specificity is at least 96%;
62. at least the CpGs defined by SEQ ID NOs 1 to 2000 and identified at nucleotide positions 61 to 62, and wherein the sensitivity is at least 29% and specificity is at least 96%;
63. at least the CpGs defined by SEQ ID NOs 1 to 3000 and identified at nucleotide positions 61 to 62, and wherein the sensitivity is at least 26% and specificity is at least 97%;
64. at least the CpGs defined by SEQ ID NOs 1 to 4000 and identified at nucleotide positions 61 to 62, and wherein the sensitivity is at least 26% and specificity is at least 97%;
65. at least the CpGs defined by SEQ ID NOs 1 to 5000 and identified at nucleotide positions 61 to 62, and wherein the sensitivity is at least 27% and specificity is at least 97%;
66. at least the CpGs defined by SEQ ID NOs 1 to 6000 and identified at nucleotide positions 61 to 62, and wherein the sensitivity is at least 29% and specificity is at least 97%;
67. at least the CpGs defined by SEQ ID NOs 1 to 7000 and identified at nucleotide positions 61 to 62, and wherein the sensitivity is at least 28% and specificity is at least 97%;
68. at least the CpGs defined by SEQ ID NOs 1 to 8000 and identified at nucleotide positions 61 to 62, and wherein the sensitivity is at least 27% and specificity is at least 97%;
69. at least the CpGs defined by SEQ ID NOs 1 to 9000 and identified at nucleotide positions 61 to 62, and wherein the sensitivity is at least 25% and specificity is at least 97%;
70. at least the CpGs defined by SEQ ID NOs 1 to 10000 and identified at nucleotide positions 61 to 62, and wherein the sensitivity is at least 25% and specificity is at least 97%;
71. at least the CpGs defined by SEQ ID NOs 1 to 11000 and identified at nucleotide positions 61 to 62, and wherein the sensitivity is at least 26% and specificity is at least 97%;
72. at least the CpGs defined by SEQ ID NOs 1 to 12000 and identified at nucleotide positions 61 to 62, and wherein the sensitivity is at least 26% and specificity is at least 97%;
73. at least the CpGs defined by SEQ ID NOs 1 to 13000 and identified at nucleotide positions 61 to 62, and wherein the sensitivity is at least 26% and specificity is at least 97%;
74. at least the CpGs defined by SEQ ID NOs 1 to 14000 and identified at nucleotide positions 61 to 62, and wherein the sensitivity is at least 25% and specificity is at least 97%;
75. at least the CpGs defined by SEQ ID NOs 1 to 15000 and identified at nucleotide positions 61 to 62, and wherein the sensitivity is at least 25% and specificity is at least 97%;
76. at least the CpGs defined by SEQ ID NOs 1 to 16000 and identified at nucleotide positions 61 to 62, and wherein the sensitivity is at least 25% and specificity is at least 97%;
77. at least the CpGs defined by SEQ ID NOs 1 to 17000 and identified at nucleotide positions 61 to 62, and wherein the sensitivity is at least 25% and specificity is at least 97%;
78. at least the CpGs defined by SEQ ID NOs 1 to 18000 and identified at nucleotide positions 61 to 62, and wherein the sensitivity is at least 25% and specificity is at least 97%;
79. at least the CpGs defined by SEQ ID NOs 1 to 19000 and identified at nucleotide positions 61 to 62, and wherein the sensitivity is at least 24% and specificity is at least 97%;
80. at least the CpGs defined by SEQ ID NOs 1 to 20000 and identified at nucleotide positions 61 to 62, and wherein the sensitivity is at least 24% and specificity is at least 97%;
81. at least the CpGs defined by SEQ ID NOs 1 to 21000 and identified at nucleotide positions 61 to 62, and wherein the sensitivity is at least 24% and specificity is at least 97%;
82. at least the CpGs defined by SEQ ID NOs 1 to 22000 and identified at nucleotide positions 61 to 62, and wherein the sensitivity is at least 24% and specificity is at least 97%;
83. at least the CpGs defined by SEQ ID NOs 1 to 23000 and identified at nucleotide positions 61 to 62, and wherein the sensitivity is at least 24% and specificity is at least 97%;
84. at least the CpGs defined by SEQ ID NOs 1 to 24000 and identified at nucleotide positions 61 to 62, and wherein the sensitivity is at least 24% and specificity is at least 97%;
85. at least the CpGs defined by SEQ ID NOs 1 to 25000 and identified at nucleotide positions 61 to 62, and wherein the sensitivity is at least 24% and specificity is at least 97%;
86. at least the CpGs defined by SEQ ID NOs 1 to 26000 and identified at nucleotide positions 61 to 62, and wherein the sensitivity is at least 24% and specificity is at least 97%;
87. at least the CpGs defined by SEQ ID NOs 1 to 27000 and identified at nucleotide positions 61 to 62, and wherein the sensitivity is at least 24% and specificity is at least 97%;
88. at least the CpGs defined by SEQ ID NOs 1 to 28000 and identified at nucleotide positions 61 to 62, and wherein the sensitivity is at least 24% and specificity is at least 97%;
89. at least the CpGs defined by SEQ ID NOs 1 to 29000 and identified at nucleotide positions 61 to 62, and wherein the sensitivity is at least 24% and specificity is at least 97%;
90. at least the CpGs defined by SEQ ID NOs 28,001 to 29,000 and identified at nucleotide positions 61 to 62, and wherein the sensitivity is at least 23% and specificity is at least 89%;
91. at least the CpGs defined by SEQ ID NOs 27,001 to 29,000 and identified at nucleotide positions 61 to 62, and wherein the sensitivity is at least 25% and specificity is at least 88%;
92. at least the CpGs defined by SEQ ID NOs 26,001 to 29,000 and identified at nucleotide positions 61 to 62, and wherein the sensitivity is at least 25% and specificity is at least 88%;
93. at least the CpGs defined by SEQ ID NOs 25,001 to 29,000 and identified at nucleotide positions 61 to 62, and wherein the sensitivity is at least 25% and specificity is at least 88%;
94. at least the CpGs defined by SEQ ID NOs 24,001 to 29,000 and identified at nucleotide positions 61 to 62, and wherein the sensitivity is at least 24% and specificity is at least 88%;
95. at least the CpGs defined by SEQ ID NOs 23,001 to 29,000 and identified at nucleotide positions 61 to 62, and wherein the sensitivity is at least 25% and specificity is at least 88%;
96. at least the CpGs defined by SEQ ID NOs 22,001 to 29,000 and identified at nucleotide positions 61 to 62, and wherein the sensitivity is at least 25% and specificity is at least 88%;
97. at least the CpGs defined by SEQ ID NOs 21,001 to 29,000 and identified at nucleotide positions 61 to 62, and wherein the sensitivity is at least 25% and specificity is at least 88%;
98. at least the CpGs defined by SEQ ID NOs 20,001 to 29,000 and identified at nucleotide positions 61 to 62, and wherein the sensitivity is at least 25% and specificity is at least 88%;
99. at least the CpGs defined by SEQ ID NOs 19,001 to 29,000 and identified at nucleotide positions 61 to 62, and wherein the sensitivity is at least 25% and specificity is at least 89%;
100. at least the CpGs defined by SEQ ID NOs 18,001 to 29,000 and identified at nucleotide positions 61 to 62, and wherein the sensitivity is at least 25% and specificity is at least 89%;
101. at least the CpGs defined by SEQ ID NOs 17,001 to 29,000 and identified at nucleotide positions 61 to 62, and wherein the sensitivity is at least 25% and specificity is at least 89%;
102. at least the CpGs defined by SEQ ID NOs 16,001 to 29,000 and identified at nucleotide positions 61 to 62, and wherein the sensitivity is at least 25% and specificity is at least 88%;
103. at least the CpGs defined by SEQ ID NOs 15,001 to 29,000 and identified at nucleotide positions 61 to 62, and wherein the sensitivity is at least 25% and specificity is at least 88%;
104. at least the CpGs defined by SEQ ID NOs 14,001 to 29,000 and identified at nucleotide positions 61 to 62, and wherein the sensitivity is at least 25% and specificity is at least 88%;
105. at least the CpGs defined by SEQ ID NOs 13,001 to 29,000 and identified at nucleotide positions 61 to 62, and wherein the sensitivity is at least 25% and specificity is at least 89%;
106. at least the CpGs defined by SEQ ID NOs 12,001 to 29,000 and identified at nucleotide positions 61 to 62, and wherein the sensitivity is at least 25% and specificity is at least 89%;
107. at least the CpGs defined by SEQ ID NOs 11,001 to 29,000 and identified at nucleotide positions 61 to 62, and wherein the sensitivity is at least 26% and specificity is at least 89%;
108. at least the CpGs defined by SEQ ID NOs 10,001 to 29,000 and identified at nucleotide positions 61 to 62, and wherein the sensitivity is at least 27% and specificity is at least 89%;
109. at least the CpGs defined by SEQ ID NOs 9,001 to 29,000 and identified at nucleotide positions 61 to 62, and wherein the sensitivity is at least 27% and specificity is at least 89%;
110. at least the CpGs defined by SEQ ID NOs 8,001 to 29,000 and identified at nucleotide positions 61 to 62, and wherein the sensitivity is at least 26% and specificity is at least 90%;
111. at least the CpGs defined by SEQ ID NOs 7,001 to 29,000 and identified at nucleotide positions 61 to 62, and wherein the sensitivity is at least 26% and specificity is at least 89%;
112. at least the CpGs defined by SEQ ID NOs 6,001 to 29,000 and identified at nucleotide positions 61 to 62, and wherein the sensitivity is at least 25% and specificity is at least 90%;
113. at least the CpGs defined by SEQ ID NOs 5,001 to 29,000 and identified at nucleotide positions 61 to 62, and wherein the sensitivity is at least 26% and specificity is at least 90%;
114. at least the CpGs defined by SEQ ID NOs 4,001 to 29,000 and identified at nucleotide positions 61 to 62, and wherein the sensitivity is at least 29% and specificity is at least 90%;
115. at least the CpGs defined by SEQ ID NOs 3,001 to 29,000 and identified at nucleotide positions 61 to 62, and wherein the sensitivity is at least 29% and specificity is at least 91%;
116. at least the CpGs defined by SEQ ID NOs 2,001 to 29,000 and identified at nucleotide positions 61 to 62, and wherein the sensitivity is at least 29% and specificity is at least 93%;
117. at least the CpGs defined by SEQ ID NOs 1,001 to 29,000 and identified at nucleotide positions 61 to 62, and wherein the sensitivity is at least 29% and specificity is at least 95%;
118. at least the CpGs defined by SEQ ID NOs 501 to 29,000 and identified at nucleotide positions 61 to 62, and wherein the sensitivity is at least 28% and specificity is at least 96%;
119. at least the CpGs defined by SEQ ID NOs 101 to 29,000 and identified at nucleotide positions 61 to 62, and wherein the sensitivity is at least 23% and specificity is at least 97%; or
120. at least the CpGs defined by SEQ ID NOs 1 to 29,000 and identified at nucleotide positions 61 to 62, and wherein the sensitivity is at least 24% and specificity is at least 97%.

In any of the described assays, the methylation status of the one or more CpGs in the panel is preferably determined by a β-value analysis, and the cancer is breast cancer or ovarian cancer. Preferably, the cancer is ovarian cancer.

In any of the assays described herein, the sensitivity and specificity of the cancer index threshold values vary depending on the number of CpGs comprised within the set, and specifically what CpGs are comprised within the set. Tables 2, 3, and 4 exemplify this assertion.

In any of the assays of the invention described herein, the individual may be stratified according to their cancer index value, and consequently be defined according to their cancer status and/or cancer risk. In any of the assays described herein, wherein when the cancer index value for the individual is:
a. less than about −0.580 the individual is assessed as not having cancer;
b. about −0.580 or more and less than about −0.280 the individual is assessed as having a low risk of cancer;
c. about −0.280 or more and less than about 0.070 the individual is assessed as having a moderate risk of cancer;
d. about 0.070 or more the individual is assessed as having a high risk of cancer;

preferably wherein the assay comprises determining methylation β-values for each CpG in the panel of one or more CpGs, more preferably wherein the cancer is breast cancer.

TABLE 2

| | AUC | | | | | | |
|---|---|---|---|---|---|---|---|
| | (internal | Sensitivity | Specificty | Sensitivity | Specificty | Sensitivity | Specificty |
| subset | validation) | (q = 0.743) | (q = 0.743) | (q = 0.126) | (q = 0.126) | (q = −0.278) | (q = −0.278) |
| 1-29000 | 0.84 | 0.24 | 0.97 | 0.73 | 0.8 | 0.94 | 0.5 |
| 101-29000 | 0.84 | 0.23 | 0.97 | 0.76 | 0.81 | 0.94 | 0.51 |
| 501-29000 | 0.83 | 0.28 | 0.96 | 0.75 | 0.77 | 0.93 | 0.52 |
| 1001-29000 | 0.8 | 0.29 | 0.95 | 0.69 | 0.76 | 0.89 | 0.52 |
| 2001-29000 | 0.77 | 0.29 | 0.93 | 0.61 | 0.73 | 0.86 | 0.53 |
| 3001-29000 | 0.75 | 0.29 | 0.91 | 0.59 | 0.73 | 0.83 | 0.53 |
| 4001-29000 | 0.72 | 0.29 | 0.9 | 0.56 | 0.72 | 0.79 | 0.53 |
| 5001-29000 | 0.71 | 0.26 | 0.9 | 0.54 | 0.72 | 0.78 | 0.53 |
| 6001-29000 | 0.69 | 0.25 | 0.9 | 0.53 | 0.72 | 0.71 | 0.53 |
| 7001-29000 | 0.66 | 0.26 | 0.89 | 0.5 | 0.72 | 0.65 | 0.52 |
| 8001-29000 | 0.68 | 0.26 | 0.9 | 0.54 | 0.71 | 0.71 | 0.53 |
| 9001-29000 | 0.66 | 0.27 | 0.89 | 0.48 | 0.72 | 0.64 | 0.52 |
| 10001-29000 | 0.66 | 0.27 | 0.89 | 0.5 | 0.71 | 0.65 | 0.52 |
| 11001-29000 | 0.64 | 0.26 | 0.89 | 0.46 | 0.72 | 0.63 | 0.53 |
| 12001-29000 | 0.64 | 0.25 | 0.89 | 0.46 | 0.72 | 0.63 | 0.53 |
| 13001-29000 | 0.64 | 0.25 | 0.89 | 0.46 | 0.72 | 0.63 | 0.53 |
| 14001-29000 | 0.63 | 0.25 | 0.88 | 0.45 | 0.72 | 0.63 | 0.53 |
| 15001-29000 | 0.63 | 0.25 | 0.88 | 0.45 | 0.72 | 0.63 | 0.53 |
| 16001-29000 | 0.63 | 0.25 | 0.88 | 0.45 | 0.72 | 0.64 | 0.54 |
| 17001-29000 | 0.62 | 0.25 | 0.89 | 0.43 | 0.72 | 0.63 | 0.53 |
| 18001-29000 | 0.62 | 0.25 | 0.89 | 0.42 | 0.72 | 0.62 | 0.53 |
| 19001-29000 | 0.62 | 0.25 | 0.89 | 0.43 | 0.72 | 0.62 | 0.53 |
| 20001-29000 | 0.63 | 0.25 | 0.88 | 0.45 | 0.72 | 0.64 | 0.52 |
| 21001-29000 | 0.61 | 0.25 | 0.88 | 0.41 | 0.72 | 0.63 | 0.51 |
| 22001-29000 | 0.62 | 0.25 | 0.88 | 0.43 | 0.73 | 0.63 | 0.51 |
| 23001-29000 | 0.62 | 0.25 | 0.88 | 0.43 | 0.73 | 0.64 | 0.51 |
| 24001-29000 | 0.61 | 0.24 | 0.88 | 0.4 | 0.72 | 0.62 | 0.52 |
| 25001-29000 | 0.6 | 0.25 | 0.88 | 0.42 | 0.72 | 0.62 | 0.51 |
| 26001-29000 | 0.62 | 0.25 | 0.88 | 0.44 | 0.71 | 0.62 | 0.52 |
| 27001-29000 | 0.61 | 0.25 | 0.88 | 0.42 | 0.73 | 0.64 | 0.52 |
| 28001-29000 | 0.61 | 0.23 | 0.89 | 0.41 | 0.74 | 0.62 | 0.54 |

TABLE 3

| | AUC | | | | | | |
|---|---|---|---|---|---|---|---|
| | (internal | Sensitivity | Specificty | Sensitivity | Specificty | Sensitivity | Specificty |
| subset | validation) | (q = 0.743) | (q = 0.743) | (q = 0.126) | (q = 0.126) | (q = −0.278) | (q = −0.278) |
| 1-100 | 0.72 | 0.35 | 0.88 | 0.66 | 0.64 | 0.84 | 0.44 |
| 1-500 | 0.78 | 0.3 | 0.95 | 0.7 | 0.72 | 0.87 | 0.5 |
| 1-1000 | 0.81 | 0.31 | 0.96 | 0.7 | 0.78 | 0.89 | 0.49 |
| 1-2000 | 0.82 | 0.29 | 0.96 | 0.74 | 0.78 | 0.89 | 0.48 |
| 1-3000 | 0.83 | 0.26 | 0.97 | 0.75 | 0.77 | 0.92 | 0.49 |
| 1-4000 | 0.83 | 0.26 | 0.97 | 0.72 | 0.77 | 0.92 | 0.49 |
| 1-5000 | 0.84 | 0.27 | 0.97 | 0.73 | 0.79 | 0.93 | 0.5 |
| 1-6000 | 0.84 | 0.29 | 0.97 | 0.72 | 0.79 | 0.93 | 0.51 |
| 1-7000 | 0.84 | 0.28 | 0.97 | 0.72 | 0.78 | 0.93 | 0.5 |
| 1-8000 | 0.84 | 0.27 | 0.97 | 0.72 | 0.79 | 0.93 | 0.49 |
| 1-9000 | 0.84 | 0.25 | 0.97 | 0.72 | 0.78 | 0.93 | 0.5 |
| 1-10000 | 0.84 | 0.25 | 0.97 | 0.73 | 0.79 | 0.93 | 0.49 |
| 1-11000 | 0.84 | 0.26 | 0.97 | 0.73 | 0.78 | 0.93 | 0.5 |
| 1-12000 | 0.84 | 0.26 | 0.97 | 0.73 | 0.78 | 0.93 | 0.48 |
| 1-13000 | 0.84 | 0.26 | 0.97 | 0.73 | 0.79 | 0.94 | 0.48 |
| 1-14000 | 0.84 | 0.25 | 0.97 | 0.73 | 0.79 | 0.93 | 0.49 |
| 1-15000 | 0.84 | 0.25 | 0.97 | 0.73 | 0.79 | 0.94 | 0.48 |
| 1-16000 | 0.84 | 0.25 | 0.97 | 0.73 | 0.8 | 0.94 | 0.49 |
| 1-17000 | 0.84 | 0.25 | 0.97 | 0.73 | 0.8 | 0.94 | 0.48 |
| 1-18000 | 0.84 | 0.25 | 0.97 | 0.73 | 0.8 | 0.94 | 0.49 |
| 1-19000 | 0.84 | 0.24 | 0.97 | 0.73 | 0.8 | 0.94 | 0.49 |
| 1-20000 | 0.84 | 0.24 | 0.97 | 0.73 | 0.79 | 0.94 | 0.49 |
| 1-21000 | 0.84 | 0.24 | 0.97 | 0.73 | 0.79 | 0.94 | 0.49 |
| 1-22000 | 0.84 | 0.24 | 0.97 | 0.73 | 0.79 | 0.94 | 0.49 |
| 1-23000 | 0.84 | 0.24 | 0.97 | 0.73 | 0.79 | 0.94 | 0.49 |
| 1-24000 | 0.84 | 0.24 | 0.97 | 0.73 | 0.8 | 0.94 | 0.49 |
| 1-25000 | 0.84 | 0.24 | 0.97 | 0.73 | 0.8 | 0.94 | 0.49 |
| 1-26000 | 0.84 | 0.24 | 0.97 | 0.73 | 0.8 | 0.94 | 0.49 |
| 1-27000 | 0.84 | 0.24 | 0.97 | 0.73 | 0.8 | 0.94 | 0.49 |
| 1-28000 | 0.84 | 0.24 | 0.97 | 0.73 | 0.8 | 0.94 | 0.49 |
| 1-29000 | 0.84 | 0.24 | 0.97 | 0.73 | 0.8 | 0.94 | 0.5 |

TABLE 4

| | AUC | | | | | | |
|---|---|---|---|---|---|---|---|
| | (internal | Sensitivity | Specificty | Sensitivity | Specificty | Sensitivity | Specificty |
| subset | validation) | (q = 0.743) | (q = 0.743) | (q = 0.126) | (q = 0.126) | (q = −0.278) | (q = −0.278) |
| 1-500 | 0.78 | 0.3 | 0.95 | 0.7 | 0.72 | 0.87 | 0.5 |
| 501-1000 | 0.78 | 0.3 | 0.93 | 0.71 | 0.74 | 0.87 | 0.5 |
| 1001-1500 | 0.77 | 0.31 | 0.91 | 0.67 | 0.74 | 0.85 | 0.5 |
| 1501-2000 | 0.74 | 0.29 | 0.92 | 0.68 | 0.68 | 0.82 | 0.45 |
| 2001-2500 | 0.77 | 0.35 | 0.9 | 0.71 | 0.72 | 0.86 | 0.49 |
| 2501-3000 | 0.72 | 0.33 | 0.89 | 0.61 | 0.71 | 0.83 | 0.47 |
| 3001-3500 | 0.75 | 0.34 | 0.93 | 0.64 | 0.75 | 0.82 | 0.53 |
| 3501-4000 | 0.7 | 0.29 | 0.91 | 0.56 | 0.7 | 0.78 | 0.48 |
| 4001-4500 | 0.74 | 0.33 | 0.9 | 0.61 | 0.69 | 0.82 | 0.48 |
| 4501-5000 | 0.76 | 0.36 | 0.92 | 0.66 | 0.72 | 0.8 | 0.51 |
| 5001-5500 | 0.74 | 0.3 | 0.88 | 0.74 | 0.69 | 0.87 | 0.51 |
| 5501-6000 | 0.72 | 0.35 | 0.9 | 0.62 | 0.71 | 0.75 | 0.51 |
| 6001-6500 | 0.72 | 0.28 | 0.92 | 0.57 | 0.69 | 0.77 | 0.5 |
| 6501-7000 | 0.71 | 0.3 | 0.92 | 0.65 | 0.69 | 0.77 | 0.49 |
| 7001-7500 | 0.71 | 0.29 | 0.89 | 0.55 | 0.69 | 0.8 | 0.51 |
| 7501-8000 | 0.69 | 0.26 | 0.86 | 0.59 | 0.68 | 0.77 | 0.49 |
| 8001-8500 | 0.72 | 0.29 | 0.89 | 0.62 | 0.7 | 0.79 | 0.5 |
| 8501-9000 | 0.72 | 0.29 | 0.91 | 0.59 | 0.73 | 0.79 | 0.51 |
| 9001-9500 | 0.72 | 0.28 | 0.89 | 0.59 | 0.71 | 0.78 | 0.54 |
| 9501-10000 | 0.7 | 0.34 | 0.87 | 0.54 | 0.71 | 0.71 | 0.52 |
| 10001-10500 | 0.68 | 0.27 | 0.87 | 0.52 | 0.72 | 0.71 | 0.53 |
| 10501-11000 | 0.69 | 0.28 | 0.88 | 0.54 | 0.72 | 0.73 | 0.52 |
| 11001-11500 | 0.68 | 0.3 | 0.87 | 0.55 | 0.69 | 0.69 | 0.54 |
| 11501-12000 | 0.64 | 0.24 | 0.88 | 0.48 | 0.7 | 0.64 | 0.54 |
| 12001-12500 | 0.67 | 0.28 | 0.89 | 0.54 | 0.68 | 0.7 | 0.5 |
| 12501-13000 | 0.68 | 0.3 | 0.89 | 0.51 | 0.72 | 0.66 | 0.55 |
| 13001-13500 | 0.69 | 0.29 | 0.89 | 0.55 | 0.72 | 0.72 | 0.55 |
| 13501-14000 | 0.67 | 0.26 | 0.89 | 0.5 | 0.73 | 0.69 | 0.52 |
| 14001-14500 | 0.66 | 0.26 | 0.88 | 0.48 | 0.73 | 0.64 | 0.55 |
| 14501-15000 | 0.63 | 0.24 | 0.88 | 0.38 | 0.73 | 0.62 | 0.54 |
| 15001-15500 | 0.69 | 0.24 | 0.89 | 0.54 | 0.71 | 0.75 | 0.52 |
| 15501-16000 | 0.64 | 0.11 | 0.72 | 0.46 | 0.39 | 0.56 | 0.24 |
| 16001-16500 | 0.64 | 0.13 | 0.73 | 0.44 | 0.4 | 0.62 | 0.25 |
| 16501-17000 | 0.66 | 0.12 | 0.7 | 0.38 | 0.4 | 0.58 | 0.23 |
| 17001-17500 | 0.61 | 0.15 | 0.74 | 0.46 | 0.38 | 0.66 | 0.23 |
| 17501-18000 | 0.64 | 0.15 | 0.75 | 0.42 | 0.38 | 0.59 | 0.24 |
| 18001-18500 | 0.61 | 0.15 | 0.75 | 0.44 | 0.39 | 0.65 | 0.23 |
| 18501-19000 | 0.62 | 0.17 | 0.73 | 0.45 | 0.41 | 0.62 | 0.22 |
| 19001-19500 | 0.62 | 0.12 | 0.74 | 0.48 | 0.38 | 0.61 | 0.23 |
| 19501-20000 | 0.62 | 0.16 | 0.7 | 0.46 | 0.41 | 0.6 | 0.22 |
| 20001-20500 | 0.64 | 0.12 | 0.71 | 0.41 | 0.4 | 0.62 | 0.21 |
| 20501-21000 | 0.62 | 0.15 | 0.72 | 0.46 | 0.42 | 0.62 | 0.25 |
| 21001-21500 | 0.62 | 0.15 | 0.72 | 0.46 | 0.39 | 0.62 | 0.21 |
| 21501-22000 | 0.63 | 0.12 | 0.74 | 0.43 | 0.39 | 0.62 | 0.22 |
| 22001-22500 | 0.64 | 0.12 | 0.72 | 0.38 | 0.41 | 0.58 | 0.22 |
| 22501-23000 | 0.61 | 0.17 | 0.74 | 0.45 | 0.39 | 0.62 | 0.24 |
| 23001-23500 | 0.59 | 0.21 | 0.76 | 0.47 | 0.4 | 0.64 | 0.21 |
| 23501-24000 | 0.6 | 0.18 | 0.73 | 0.5 | 0.37 | 0.63 | 0.23 |
| 24001-24500 | 0.64 | 0.13 | 0.72 | 0.42 | 0.39 | 0.62 | 0.22 |
| 24501-25000 | 0.6 | 0.16 | 0.72 | 0.5 | 0.37 | 0.63 | 0.19 |
| 25001-25500 | 0.58 | 0.19 | 0.75 | 0.46 | 0.4 | 0.69 | 0.24 |
| 25501-26000 | 0.6 | 0.19 | 0.75 | 0.45 | 0.44 | 0.62 | 0.23 |
| 26001-26500 | 0.59 | 0.16 | 0.75 | 0.5 | 0.41 | 0.66 | 0.24 |
| 26501-27000 | 0.61 | 0.2 | 0.72 | 0.44 | 0.39 | 0.62 | 0.23 |
| 27001-27500 | 0.56 | 0.24 | 0.77 | 0.48 | 0.45 | 0.68 | 0.22 |
| 27501-28000 | 0.62 | 0.16 | 0.75 | 0.44 | 0.4 | 0.63 | 0.23 |
| 28001-28500 | 0.6 | 0.21 | 0.73 | 0.48 | 0.4 | 0.65 | 0.21 |
| 28501-29000 | 0.61 | 0.17 | 0.77 | 0.48 | 0.39 | 0.63 | 0.21 |

### Relationship Between Cancer Index Value and Determining Methylation Status of CpGs

In view of the observations described herein (see Examples), the inventors derived a cancer index based on an analysis of methylation status (DNAme; as described above) for use in assays for assessing the presence or development of cancer in an individual.

As explained herein, the described assays particularly relate to the assessment of assessing the presence, absence or development of breast cancer and/or ovarian cancer, particularly breast cancer.

Any of the assays described herein involve deriving a cancer index value based on the methylation of status of a panel of one or more CpGs assayed in a sample provided from an individual, as described and defined herein.

The cancer index value may be derived by any suitable means.

The inventors have identified specific CpGs, as described and defined herein, which may be used to form a panel of CpGs whose methylation status is determined in order to establish cancer index values in accordance with the assays described and defined herein. Using these panels the inventors have demonstrated that it is possible to derive a cancer index value which correlates with and is indicative of normal tissue, i.e. tissue which is cancer negative, in particular breast and/or ovarian tissue which is cancer negative. Accordingly, cancer can be assessed to be absent in the individual. Using these panels the inventors have demonstrated that it is possible to derive a cancer index value which correlates with and is indicative of cancer tissue, i.e. tissue which is cancer positive, in particular breast and/or ovarian tissue which is cancer negative. Accordingly, cancer can be assessed to be present in the individual. As explained herein, the inventors have shown that using panels of the CpGs that have been identified it can be shown that the DNA methylation profile of normal cells derived from an anatomical site other than the breast or ovary, such as the cervix, the vagina, the buccal area, blood and/or urine, preferably cervical liquid-based cytology, more preferably tissue derived from a cervical smear sample, as indicated by the cancer index value, is dynamic and subject to change on a continuum from indicating cancer negative to cancer positive tissue. In particular, the cancer index value described herein acts as a surrogate for indicating whether the breast and/or ovarian tissue of an individual is cancer negative or cancer positive to a high degree of statistical accuracy. As such, using panels of the CpGs that have been identified it is possible to establish a cancer index value scale that can be used to assess the presence, absence or development of cancer in an individual.

As described herein, the inventors have used certain methods for determining the methylation status of specific CpGs in the population of DNA molecules in the sample. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

As will be apparent to a skilled person, in the assays of the invention the steps of determining the methylation status of specific CpGs in the population of DNA molecules in the sample are not limited to any one specific methodology. As the skilled person will appreciate, because the cancer index value is based on the methylation status of CpGs, and since the methylation status of CpGs can be represented by values which may be specific to a specific methodology, e.g. percent methylated reference (PMR) value or methylation β-value, then the range of cancer index values which define cancer negative and cancer positive samples may be dependent upon the methodology used to determine the methylation status of CpGs. Nevertheless, a user may readily reproduce and implement the assays of the invention using any suitable methodology for determining the methylation status of CpGs, provided that the same methodology is used consistently. Moreover, the user can readily establish, de novo, cancer index values which define cancer negative and cancer positive samples by determining the methylation status of CpGs in panels constituting the specific CpGs disclosed herein from known cancer negative and cancer positive patient samples. Once such cancer index values are established using the CpGs identified herein, a user may use these values as a basis for assessing the presence, absence or development of cancer in any test individual whose cancer status is to be determined. Accordingly, cancer index values according to the present invention are not limited to specific methods of determination of methylation status of CpGs. On the contrary, the skilled person will appreciate that cancer index values can be established which reflect the intrinsic capabilities of the CpGs identified herein to correlate methylation status with cancer disease status.

Accordingly, the cancer index value may be derived by assessing the methylation status of the one or more CpGs in the panel in a sample provided from an individual by any suitable means.

The step of determining the methylation status of each CpG in the panel of one or more CpGs may be achieved by determining a percent methylated reference (PMR) value of each one of the one or more CpGs. The step of determining the methylation status of each CpG in the panel of one or more CpGs may be achieved by determining the methylation β-value of each one of the one or more CpGs.

In any of the assays described herein, the methylation status of the CpGs may be determined by any suitable means. For example, in any of the assays described herein the step of determining the methylation status of each CpG in the panel of one or more CpGs may comprise:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

The step of determining the methylation status of each CpG in the panel of one or more CpGs may comprise a conversion step in order to distinguish methylated CpG dinucleotides relative to non-methylated CpG dinucleotides. The conversion step may comprise e.g. bisulfate conversion or TAPS (TET-assisted pyridine borane sequencing) conversion of the DNA in a sample that is to be applied to any one or more of a. to c. above. TAPS may particularly involve the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

The step of determining the methylation status of each CpG in the panel of one or more CpGs may additionally, or alternatively, comprise the use of TempO-seq (templated Olig-sequencing). The oligoniclueotides in the context of TempO-seq may or may not be designed such that they hybridise with methylated CpG dinucleotides following a prior conversion as described herein.

The step of determining the methylation status of each CpG in the panel of one or more CpGs may comprise the contacting the DNA in the sample with one or more methylation sensitive restriction endonucleases that cleave methylated and/or unmethylated forms of their restriction sites, and preferably the contacting of the DNA is prior to performing any one of a. to c. above. In assays of the invention wherein methylation sensitive restriction enzymes are used, one or more control reactions are performed. Preferably, the one or more control reactions involve interrogation of known loci that contain (i) no restriction endonuclease sites; (ii) a restriction site that is methylated; (iii) a restriction site that is unmethylated.

Using any of the methods for determining the methylation status of each CpG in the panel of one or more CpGs, the proportion of methylated and unmethylated CpGs at any given locus may be determined, thereby enabling generation of a cancer index value.

Preferably, the the step of determining in the population of DNA molecules in the sample the methylation status of a panel of one or more CpGs further comprises determining a R value of each CpG. Deriving the cancer index value may involve providing a methylation β-value data set comprising the methylation β-values for each CpG in the panel of one or more CpGs.

### Assessment of Methylation Status of CpGs

Methylation of DNA is a recognised form of epigenetic modification which has the capability of altering the expression of genes and other elements such as microRNAs. In cancer development and progression, methylation may have the effect of e.g. silencing tumor suppressor genes and/or increasing the expression of oncogenes. Other forms of dysregulation may occur as a result of methylation. Methylation of DNA occurs at discrete loci which are predominately dinucleotides consisting of a CpG motif, but may also occur at CHH motifs (where H is A, C, or T). During methylation, a methyl group is added to the fifth carbon of cytosine bases to create methylcytosine.

Methylation can occur throughout the genome and is not limited to regions with respect to an expressed sequence such as a gene. Methylation typically, but not always, occurs in a promoter or other regulatory region of an expressed sequence such as enhancer elements. Most typically, the methylation status of CpGs is clustered in CpG islands, for example CpG islands present in the regulatory regions of genes, especially in their promoter regions.

Typically, an assessment of DNA methylation status involves analysing the presence or absence of methyl groups in DNA, for example methyl groups on the 5 position of one or more cytosine nucleotides. Preferably, the methylation status of one or more cytosine nucleotides present as a CpG dinucleotide (where C stands for Cytosine, G for Guanine and p for the phosphate group linking the two) is assessed.

A variety of techniques are available for the identification and assessment of CpG methylation status, as will be outlined briefly below. The assays described herein encompass any suitable technique for the determination of CpG methylation status.

Methyl groups are lost from a starting DNA molecule during conventional in vitro handling steps such as PCR. To avoid this, techniques for the detection of methyl groups commonly involve the preliminary treatment of DNA prior to subsequent processing, in a way that preserves the methylation status information of the original DNA molecule. Such preliminary techniques involve three main categories of processing, i.e. bisulphite modification, restriction enzyme digestion and affinity-based analysis. Products of these techniques can then be coupled with sequencing or array-based platforms for subsequent identification or qualitative assessment of CpG methylation status.

Techniques involving bisulphite modification of DNA have become the most common assays for detection and assessment of methylation status of CpG dinucleotides. Treatment of DNA with bisulphite, e.g. sodium bisulphite, converts cytosine bases to uracil bases, but has no effect on 5-methylcytosines. Thus, the presence of a cytosine in bisulphite-treated DNA is indicative of the presence of a cytosine base which was previously methylated in the starting DNA molecule. Such cytosine bases can be detected by a variety of techniques. For example, primers specific for unmethylated versus methylated DNA can be generated and used for PCR-based identification of methylated CpG dinucleotides. DNA may be amplified, either before or after bisulphite conversion. A separation/capture step may be performed, e.g. using binding molecules such as complementary oligonucleotide sequences. Standard and next-generation DNA sequencing protocols can also be used.

In other approaches, methylation-sensitive enzymes can be employed which digest or cut only in the presence of methylated DNA. Analysis of resulting fragments is commonly carried out using mircroarrays.

Affinity-based techniques exploit binding interactions to capture fragments of methylated DNA for the purposes of enrichment. Binding molecules such as anti-5-methylcytosine antibodies are commonly employed prior to subsequent processing steps such as PCR and sequencing.

Olkhov-Mitsel and Bapat (2012) provide a comprehensive review of techniques available for the identification and assessment of biomarkers involving methylcytosine.

For the purposes of assessing the methylation status of the CpG-based biomarkers characterised and described herein, any suitable assay can be employed.

Assays described herein may comprise determining methylation status of CpGs by bisulphite converting the DNA. Preferred assays involve bisulphite treatment of DNA, including amplification of the identified CpG loci for methylation specific PCR and/or sequencing and/or assessment of the methylation status of target loci using methylation-discriminatory microarrays.

Amplification of CpG loci can be achieved by a variety of approaches. Preferably, CpG loci are amplified using PCR. A variety of PCR-based approaches may be used. For example, methylation-specific primers may be hybridized to DNA containing the CpG sequence of interest. Such primers may be designed to anneal to a sequence derived from either a methylated or non-methylated CpG locus. Following annealing, a PCR reaction is performed and the presence of a subsequent PCR product indicates the presence of an annealed CpG of identifiable sequence. In such assays, DNA is bisulphite converted prior to amplification. Such techniques are commonly referred to as methylation specific PCR (MSP).

In other techniques, PCR primers may anneal to the CpG sequence of interest independently of the methylation status, and further processing steps may be used to determine the status of the CpG. Assays are designed so that the CpG site(s) are located between primer annealing sites. This assay scheme is used in techniques such as bisulphite genomic sequencing, COBRA, Ms-SNuPE. In such assay, DNA can be bisulphite converted before or after amplification.

Small-scale PCR approaches may be used. Such approaches commonly involve mass partitioning of samples (e.g. digital PCR). These techniques offer robust accuracy and sensitivity in the context of a highly miniaturised system (pico-liter sized droplets), ideal for the subsequent handling of small quantities of DNA obtainable from the potentially small volume of cellular material present in biological samples, particularly urine samples. A variety of such small-scale PCR techniques are widely available. For example, microdroplet-based PCR instruments are available from a variety of suppliers, including RainDance Technologies, Inc. (Billerica, MA; http://raindancetech.com/) and Bio-Rad, Inc. (http://www.bio-rad.com/). Microarray platforms may also be used to carry out small-scale PCR. Such platforms may include microfluidic network-based arrays e.g. available from Fluidigm Corp. (www.fluidigm.com).

Following amplification of CpG loci, amplified PCR products may be coupled to subsequent analytical platforms in order to determine the methylation status of the CpGs of interest. For example, the PCR products may be directly sequenced to determine the presence or absence of a methylcytosine at the target CpG or analysed by array-based techniques.

Any suitable sequencing techniques may be employed to determine the sequence of target DNA. In the assays of the present invention the use of high-throughput, so-called “second generation”, “third generation” and “next generation” techniques to sequence bisulphite-treated DNA can be used.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, CA; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore (https://www.nanoporetech.com/). In an alternative assay, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

In other assays sequencing steps may be omitted. For example, amplified PCR products may be applied directly to hybridization arrays based on the principle of the annealing of two complementary nucleic acid strands to form a double-stranded molecule. Hybridization arrays may be designed to include probes which are able to hybridize to amplification products of a CpG and allow discrimination between methylated and non-methylated loci. For example, probes may be designed which are able to selectively hybridize to an CpG locus containing thymine, indicating the generation of uracil following bisulphite conversion of an unmethylated cytosine in the starting template DNA. Conversely, probes may be designed which are able to selectively hybridize to a CpG locus containing cytosine, indicating the absence of uracil conversion following bisulphite treatment. This corresponds with a methylated CpG locus in the starting template DNA.

Following the application of a suitable detection system to the array, computer-based analytical techniques can be used to determine the methylation status of a CpG. Detection systems may include, e.g. the addition of fluorescent molecules following a methylation status-specific probe extension reaction. Such techniques allow CpG status determination without the specific need for the sequencing of CpG amplification products. Such array-based discriminatory probes may be termed methylation-specific probes.

Any suitable methylation-discriminatory microarrays may be employed to assess the methylation status of the CpGs described herein. One particular methylation-discriminatory microarray system is provided by Illumina, Inc. (San Diego, CA; http://www.illumina.com/). In particular, the Infinium MethylationEPIC BeadChip array and the Infinium HumanMethylation450 BeadChip array systems may be used to assess the methylation status of CpGs for predicting cancer development as described herein. Such a system exploits the chemical modifications made to DNA following bisulphite treatment of the starting DNA molecule. Briefly, the array comprises beads to which are coupled oligonucleotide probes specific for DNA sequences corresponding to the unmethylated form of a CpG, as well as separate beads to which are coupled oligonucleotide probes specific for DNA sequences corresponding to the methylated form of an CpG. Candidate DNA molecules are applied to the array and selectively hybridize, under appropriate conditions, to the oligonucleotide probe corresponding to the relevant epigenetic form. Thus, a DNA molecule derived from a CpG which was methylated in the corresponding genomic DNA will selectively attach to the bead comprising the methylation-specific oligonucleotide probe, but will fail to attach to the bead comprising the non-methylation-specific oligonucleotide probe. Single-base extension of only the hybridized probes incorporates a labeled ddNTP, which is subsequently stained with a fluorescence reagent and imaged. The methylation status of the CpG is determined by calculating the ratio of the fluorescent signal derived from the methylated and unmethylated sites.

Infinium HumanMethylation450 BeadChip array systems can be used to interrogate CpGs in the assays described herein. Alternative or customised arrays could, however, be employed to interrogate the cancer-specific CpG biomarkers defined herein, provided that they comprise means for interrogating all CpG for a given assay, as defined herein.

Techniques involving combinations of the above-described assays may also be used. For example, DNA containing CpG sequences of interest may be hybridized to microarrays and then subjected to DNA sequencing to determine the status of the CpG as described above.

In the assays described above, sequences corresponding to CpG loci may also be subjected to an enrichment process if desired. DNA containing CpG sequences of interest may be captured by binding molecules such as oligonucleotide probes complementary to the CpG target sequence of interest. Sequences corresponding to CpG loci may be captured before or after bisulphite conversion or before or after amplification. Probes may be designed to be complementary to bisulphite converted DNA. Captured DNA may then be subjected to further processing steps to determine the status of the CpG, such as DNA sequencing steps.

Capture/separation steps may be custom designed. Alternatively a variety of such techniques are available commercially, e.g. the SureSelect target enrichment system available from Agilent Technologies (http://www.agilent.com/home). In this system biotinylated “bait” or “probe” sequences (e.g. RNA) complementary to the DNA containing CpG sequences of interest are hybridized to sample nucleic acids. Streptavidin-coated magnetic beads are then used to capture sequences of interest hybridized to bait sequences. Unbound fractions are discarded. Bait sequences are then removed (e.g. by digestion of RNA) thus providing an enriched pool of CpG target sequences separated from non-CpG sequences. Template DNA may be subjected to bisulphite conversion and target loci amplified by small-scale PCR such as microdroplet PCR using primers which are independent of the methylation status of the CpG. Following amplification, samples may be subjected to a capture step to enrich for PCR products containing the target CpG, e.g. captured and purified using magnetic beads, as described above. Following capture, a standard PCR reaction is carried out to incorporate DNA sequencing barcodes into CpG-containing amplicons. PCR products are again purified and then subjected to DNA sequencing and analysis to determine the presence or absence of a methylcytosine at the target genomic CpG.

CpG biomarker loci defined herein by SEQ ID NOs 1 to 29,000 correspond to Illumina® identifiers (IlmnID) known in the art. These CpG loci identifiers refer to individual CpG sites used in the commercially available Illumina® Infinium Methylation EPIC BeadChip kit and Illumina® Infinium Human Methylation450 BeadChip kit. The identity of each CpG site represented by each CpG loci identifier is publicly available from the Illumina, Inc. website under reference to the CpG sites used in the Infinium Methylation EPIC BeadChip kit and the Infinium Human Methylation450 BeadChip kit.

To complement evolving public databases to provide accurate CpG loci identifiers and strand orientation, Illumina® has developed a method to consistently designate CpG loci based on the actual or contextual sequence of each individual CpG locus. To unambiguously refer to CpG loci in any species, Illumina® has developed a consistent and deterministic CpG loci database to ensure uniformity in the reporting of methylation data. The Illumina® method takes advantage of sequences flanking a CpG locus to generate a unique CpG locus cluster ID. This number is based on sequence information only and is unaffected by genome version. Illumina's standardized nomenclature also parallels the TOP/BOT strand nomenclature (which indicates the strand orientation) commonly used for single nucleotide polymorphism (SNP) designation.

Illumina® Identifiers for the Infinium MethylationEPIC BeadChip and Infinium Human Methylation450 BeadChip system are also available from public repositories such as Gene Expression Omnibus (GEO) (http://www.ncbi.nlm.nih.gov/geo/). By assessing the methylation status of a CpG it is meant that a determination is made as to whether a given CpG is methylated or unmethylated. In addition, it is meant that a determination is made as to the degree to which a given CpG site is methylated across a population of CpG loci in a sample.

CpG methylation status may be measured indirectly using a detection system such as fluorescence. A methylation-discriminatory microarray may be used. When calculating the degree of methylation of a given CpG, the Illumina® definition of beta-values may be used. The Illumina® methylation beta-value of a specific CpG site is calculated from the intensity of the methylated (M) and unmethylated (U) alleles, as the ratio of fluorescent signals β=Max(M,0)/[Max(M,0)+Max(U,0)+100]. On this scale, 0<β<1, β values of 1 or close to 1 indicate 100% methylation whereas values of 0 or close to 0 indicate 0% methylation.

The methylation status of any one or more CpGs of the 29,000 CpGs defined according to SEQ ID NOS: 1 to 29,000 may be assessed by any suitable technique. As explained in more detail in the Examples below, one particular exemplary technique which the inventors have used is a methylation discriminatory array, such as an Illumina InfiniumMethylation EPIC BeadChip. These assays utilise probes directed to methylated and unmethylated CpGs at a given locus.

Another exemplary technique which the inventors have used to determine the methylation status of any one or more CpGs is a fluorescence-based PCR technique referred to as MethyLight. These assays utilise forward and reverse PCR primers specific for sequences encompassing any one or more of the 29,000 CpGs defined according to SEQ ID NOS: 1 to 29,000. The methylation status of one or more of the 29,000 CpGs defined according to SEQ ID NOS: 1 to 29,000 may therefore be determined by MethyLight analysis. The detectable probes are typically designed such that they hybridise only to methylated forms of the one or more CpGs to be assayed.

### Bioinformatic Tools and Statistical Metrics for CpG-Based Assays

Software programs which aid in the in silico analysis of bisulphite converted DNA sequences and in primer design for the purposes of methylation-specific analyses are generally available and have been described previously.

In risk models for predicting cancer, a receiver-operating-characteristic (ROC) curve analysis is often used, in which the area under the curve (AUC) is assessed. Each point on the ROC curve shows the effect of a rule for turning a risk/likelihood estimate into a prediction of the presence, absence or development of cancer in an individual.

The AUC measures how well the model discriminates between case subjects and control subjects. An ROC curve that corresponds to a random classification of case subjects and control subjects is a straight line with an AUC of 50%. An ROC curve that corresponds to perfect classification has an AUC of 100%.

In any of the methods described herein, the 95% confidence interval for the ROC AUC may be between 0.60 and 1.

In any of the methods described herein, the interval may be defined as a range having as an upper limit any number between 0.60 and 1. The upper limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval may be defined as a range having as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval range may comprise any of the above lower limit numbers combined with any of the above upper limit numbers as appropriate.

Preferably, the upper limit number is 1. Thus, the 95% confidence ROC AUC interval may be defined as a range having an upper limit of 1 and as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

The upper limit number may be 0.99. Thus, the 95% confidence ROC AUC interval may be defined as a range having an upper limit of 0.99 and as a lower limit any number between 0.60 and 0.99. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98 or 0.99.

The upper limit number may be 0.98. Thus, the 95% confidence ROC AUC interval may be defined as a range having an upper limit of 0.98 and as a lower limit any number between 0.60 and 0.98. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 or 0.98.

The upper limit number may be 0.97. Thus, the 95% confidence ROC AUC interval may be defined as a range having an upper limit of 0.97 and as a lower limit any number between 0.60 and 0.97. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96 or 0.97.

The upper limit number may be 0.96. Thus, the 95% confidence ROC AUC interval may be defined as a range having an upper limit of 0.96 and as a lower limit any number between 0.60 and 0.96. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95 or 0.96.

The upper limit number may be 0.95. Thus, the 95% confidence ROC AUC interval may be defined as a range having an upper limit of 0.95 and as a lower limit any number between 0.60 and 0.95. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94 or 0.95.

The upper limit number may be 0.94. Thus, the 95% confidence ROC AUC interval may be defined as a range having an upper limit of 0.94 and as a lower limit any number between 0.60 and 0.94. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93 or 0.94.

The upper limit number may be 0.93. Thus, the 95% confidence ROC AUC interval may be defined as a range having an upper limit of 0.93 and as a lower limit any number between 0.60 and 0.93. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92 or 0.93.

The upper limit number may be 0.92. Thus, the 95% confidence ROC AUC interval may be defined as a range having an upper limit of 0.92 and as a lower limit any number between 0.60 and 0.92. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91 or 0.92.

The upper limit number may be 0.91. Thus, the 95% confidence ROC AUC interval may be defined as a range having an upper limit of 0.91 and as a lower limit any number between 0.60 and 0.91. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90 or 0.91.

The upper limit number may be 0.90. Thus, the 95% confidence ROC AUC interval may be defined as a range having an upper limit of 0.90 and as a lower limit any number between 0.60 and 0.90. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89 or 0.90.

The upper limit number may be 0.89. Thus, the 95% confidence ROC AUC interval may be defined as a range having an upper limit of 0.89 and as a lower limit any number between 0.60 and 0.89. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88 or 0.89.

The upper limit number may be 0.88. Thus, the 95% confidence ROC AUC interval may be defined as a range having an upper limit of 0.88 and as a lower limit any number between 0.60 and 0.88. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87 or 0.88.

The upper limit number may be 0.87. Thus, the 95% confidence ROC AUC interval may be defined as a range having an upper limit of 0.87 and as a lower limit any number between 0.60 and 0.87. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86 or 0.87.

The upper limit number may be 0.86. Thus, the 95% confidence ROC AUC interval may be defined as a range having an upper limit of 0.86 and as a lower limit any number between 0.60 and 0.86. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85 or 0.86.

The upper limit number may be 0.85. Thus, the 95% confidence ROC AUC interval may be defined as a range having an upper limit of 0.85 and as a lower limit any number between 0.60 and 0.85. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84 or 0.85.

The upper limit number may be 0.84. Thus, the 95% confidence ROC AUC interval may be defined as a range having an upper limit of 0.84 and as a lower limit any number between 0.60 and 0.84. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83 or 0.84.

The upper limit number may be 0.83. Thus, the 95% confidence ROC AUC interval may be defined as a range having an upper limit of 0.83 and as a lower limit any number between 0.60 and 0.83. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82 or 0.83.

The upper limit number may be 0.82. Thus, the 95% confidence ROC AUC interval may be defined as a range having an upper limit of 0.82 and as a lower limit any number between 0.60 and 0.82. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81 or 0.82.

The upper limit number may be 0.81. Thus, the 95% confidence ROC AUC interval may be defined as a range having an upper limit of 0.81 and as a lower limit any number between 0.60 and 0.81. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80 or 0.81.

The upper limit number may be 0.80. Thus, the 95% confidence ROC AUC interval may be defined as a range having an upper limit of 0.80 and as a lower limit any number between 0.60 and 0.80. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79 or 0.80.

The upper limit number may be 0.79. Thus, the 95% confidence ROC AUC interval may be defined as a range having an upper limit of 0.79 and as a lower limit any number between 0.60 and 0.79. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78 or 0.79.

The upper limit number may be 0.78. Thus, the 95% confidence ROC AUC interval may be defined as a range having an upper limit of 0.78 and as a lower limit any number between 0.60 and 0.78. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77 or 0.78.

The upper limit number may be 0.77. Thus, the 95% confidence ROC AUC interval may be defined as a range having an upper limit of 0.77 and as a lower limit any number between 0.60 and 0.77. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76 or 0.77. The upper limit number may be 0.76. Thus, the 95% confidence ROC AUC interval may be defined as a range having an upper limit of 0.76 and as a lower limit any number between 0.60 and 0.76. The lower limit number may be 0.60, 0.61, 0.62, 0.63, 0.64, 0.65, 0.66, 0.67, 0.68, 0.69, 0.70, 0.71, 0.72, 0.73, 0.74, 0.75 or 0.76.

The upper limit number may be 0.75. Thus, the 95% confidence ROC AUC interval may be defined as a range having an upper limit of 0.75 and as a lower limit any number between 0.60 and 0.75. The lower limit number may be 0.60, 0.61, 0.62, 0.63, 0.64, 0.65, 0.66, 0.67, 0.68, 0.69, 0.70, 0.71, 0.72, 0.73, 0.74 or 0.75.

The upper limit number may be 0.74. Thus, the 95% confidence ROC AUC interval may be defined as a range having an upper limit of 0.74 and as a lower limit any number between 0.60 and 0.74. The lower limit number may be 0.60, 0.61, 0.62, 0.63, 0.64, 0.65, 0.66, 0.67, 0.68, 0.69, 0.70, 0.71, 0.72, 0.73 or 0.74.

The upper limit number may be 0.73. Thus, the 95% confidence ROC AUC interval may be defined as a range having an upper limit of 0.73 and as a lower limit any number between 0.60 and 0.73. The lower limit number may be 0.60, 0.61, 0.62, 0.63, 0.64, 0.65, 0.66, 0.67, 0.68, 0.69, 0.70, 0.71, 0.72 or 0.73.

The upper limit number may be 0.72. Thus, the 95% confidence ROC AUC interval may be defined as a range having an upper limit of 0.72 and as a lower limit any number between 0.60 and 0.72. The lower limit number may be 0.60, 0.61, 0.62, 0.63, 0.64, 0.65, 0.66, 0.67, 0.68, 0.69, 0.70, 0.71 or 0.72.

The upper limit number may be 0.71. Thus, the 95% confidence ROC AUC interval may be defined as a range having an upper limit of 0.71 and as a lower limit any number between 0.60 and 0.71. The lower limit number may be 0.60, 0.61, 0.62, 0.63, 0.64, 0.65, 0.66, 0.67, 0.68, 0.69, 0.70 or 0.71.

The upper limit number may be 0.70. Thus, the 95% confidence ROC AUC interval may be defined as a range having an upper limit of 0.70 and as a lower limit any number between 0.60 and 0.70. The lower limit number may be 0.60, 0.61, 0.62, 0.63, 0.64, 0.65, 0.66, 0.67, 0.68, 0.69 or 0.70.

Epithelial and Fat Cell Proportion in a Sample and Non-Fat Cell Differentiation Characteristics

The assays described herein may also comprise determining proportions of cell types within a sample which has been taken from an individual. The proportion of cell types may further enable prediction of the presence or development of breast cancer in an individual.

Determining the proportion of cells in any of the assays described herein may comprise utilisation of any suitable technique known in the art for determining cell identity and thus proportion of cells in a sample which has been taken from an individual. The determining the proportion of cells may involve genetic or epigenetic analysis. The determining the proportion of epithelial and/or fat cells may comprise determining cellular characteristics by gene expression profiling, non-coding RNA profiling, epigenome profiling, DNA methylation profiling, deriving a WID-BC-Index and/or immunohistochemistry. The determining the proportion of cells may involve comparing any one or more of said cellular characteristics with other specific cell types or reference datasets in order to robustly identify epithelial and/or fat cells in the sample. The determining the proportion of epithelial and/or fat cells may involve DNA methylation analysis, which may comprise comparison with reference DNA methylation profiles for specific cell types. The determining the proportion of cells may involve the use of EpiDISH and/or HEpiDISH.

Any of the assays described herein may comprise determining in the sample from the individual the proportion of epithelial cells and/or determining in the sample from the individual the proportion of fat cells.

High levels of epithelial cells within a sample taken from an individual, or increased levels of epithelial cells within two or more samples taken from an individual over time after initially assessing a first sample, may indicate an increased risk of an individual having breast cancer or developing breast cancer. Low levels of fat cells within a sample taken from an individual, or decreased levels of fat cells within two or more samples taken from an individual over time after initially assessing a first sample, may indicate an increased risk of an individual having breast cancer or developing breast cancer. High levels of epithelial cells and low levels of fat cells within a sample taken from an individual may indicate an increased risk of breast cancer in the individual.

Low levels of epithelial cells within a sample taken from an individual, or decreased levels of epithelial cells within one or more samples taken from an individual over time after initially assessing a first sample, may indicate an decreased risk of an individual having breast cancer or developing breast cancer. High levels of fat cells within a sample taken from an individual, or increased levels of fat cells within two or more samples taken from an individual over time after initially assessing a first sample, may indicate an decreased risk of an individual having breast cancer or developing breast cancer. Low levels of epithelial cells and high levels of fat cells within a sample taken from an individual may indicate an increased risk of breast cancer in the individual.

In any of the assays described herein, the assay may further comprise:
i. determining in the sample from the individual the proportion of epithelial cells;
ii. determining in the sample from the individual the proportion of fat cells; and/or
iii. determining in the sample from the individual differentiation characteristics of non-fat cells.

The present inventors have shown that increased epithelial cell proportion and decreased fat cell proportion in a sample taken from an individual can associate with at least a moderate risk of harbouring breast cancer or at least a moderate risk of breast cancer development as determined by derivation of a breast cancer index value in the individual. Particularly, the inventors have shown that the proportion of epithelial and fat cells in a sample taken from an individual may change. Fat cells and epithelial cells in the context of the assays disclosed herein may change with or without prior treatment being administered to the individual. In the assays and methods described herein, epithelial and/or fat cell proportion may be monitored for changes, particularly in response to one or more treatments. Fat cell and epithelial cell proportion in samples obtained from an individual, particularly sample of cervical and breast tissue origin, may reflect changes in breast cancer index according to any of the methods described herein. Thus, fat cell and epithelial cell proportion may equally represent an assay for predicting the presence or development of breast cancer in an individual and/or monitoring the risk of an individual harbouring breast cancer or of breast cancer development, in a likewise manner to the assays for determining a breast cancer index value in a sample from an individual described herein.

The assays described herein may comprise determining in the sample from the individual differentiation characteristics of non-fat cells. In combination with the breast cancer index values described herein, the differentiation of non-fat cells to fat cells may further enable prediction of the presence or development of breast cancer in an individual. “Differentiation characteristics” in the context of the present invention refers to cellular identity, as defined by any one or more cellular characteristics such as the cell's genomic or epigenomic characteristics. Particularly, the determining of differentiation characteristics may comprise comparing the characteristics of non-fat cells in the sample to characteristics of fat cells in order to determine if non-fat cells within the sample are undergoing differentiation to fat cells.

Determining differentiation characteristics of non-fat cells to fat cells in any of the assays described herein may comprise utilisation of any suitable technique known in the art for determining cell differentiation characteristics. The determining differentiation characteristics of non-fat cells involve genetic or epigenetic analysis. The determining differentiation characteristics of non-fat cells in the sample may comprise determining the non-fat cell characteristics by gene expression profiling, non-coding RNA profiling, epigenome profiling, DNA methylation profiling, deriving a WID-BC-Index and/or immunohistochemistry. The determining differentiation characteristics of the non-fat cells may involve comparing any one or more of said cellular characteristics with characteristics of fat cells or fat cell reference data e.g. publically available ENCODE data. The determining differentiation characteristics of non-fat cells may involve the detection of lipids in the sample by any suitable method. The determining differentiation characteristics of non-fat cells may involve DNA methylation analysis, which may comprise comparison with reference DNA methylation profiles for specific fat cell types. The determining differentiation characteristics of non-fat cells may involve RT-PCR based methods for detection of known genetic markers of fat cells. The determining the proportion of cells may involve the use of EpiDISH and/or HEpiDISH.

In any of the assays described herein, wherein the determining the proportion of epithelial and/or fat cells and/or determining differentiation characteristics of non-fat cells comprises performing a method comprising:
a. gene expression profiling;
b. non-coding RNA-profiling;
c. epigenome profiling;
d. DNA methylation profiling;
e. deriving a WID-BC-Index; and/or
f. immunohistochemistry; and
   arriving at a determination based on the results of the method

Preferably, in any of the assays described herein, the sample derived from the individual for determining changes in epithelial cell proportion, fat cell proportion and/or differentiation characteristics of non-fat cells is a sample from the breast.

### Methods of Treatment and Diagnosis

The term “treatment” as used herein is intended to refer to any intervention or procedure performed on an individual, including a surgical intervention or a pharmacological intervention such as the administration of a compound or drug. Any such treatment may be performed for therapeutic purposes or for preventative or prophylactic purposes.

The invention also encompasses the performance of one or more treatment steps following a positive classification of cancer, particularly breast and/or ovarian cancer, based on any of the methods described herein. Said treatments may be considered “therapeutic” treatments.

The invention also encompasses the performance of one or more treatment steps following a negative classification of cancer or prediction of an individual being at risk of cancer development, particularly breast and/or ovarian cancer, based on any of the methods described herein. Said treatments may be considered “risk prevention”, “preventative” or “prophylactic” treatments.

The invention also encompasses the performance of one or more treatment steps following a negative classification of cancer or prediction of an individual being at risk of cancer development based on any of the methods described herein, in an individual that harbours one or more mutations that predispose the individual to an increased risk of developing cancer, particularly breast and/or ovarian cancer, such as a BRCA1 and/or a BRCA2 mutation.

The invention thus encompasses a method of treating a cancer patient comprising administering chemotherapy, radiation, immunotherapy or any cancer therapy described herein to the patient determined to have a cancer index value which indicates that the patient has is positive for cancer based on any of the assays described herein, preferably wherein the cancer is breast cancer.

The invention thus encompasses a method of treating or preventing cancer in an individual, the method comprising:
a. assessing the cancer status of the individual by assessing the presence, absence or development of cancer in the individual by performing any one of the assays described herein;
b. administering one or more therapeutic or preventative treatments to the individual based on the assessment, preferably wherein the cancer is breast and/or ovarian cancer, most preferably

wherein the cancer is breast cancer.

The invention thus encompasses a method of treating or preventing cancer in an individual, the method comprising:
a. assessing the cancer status of the individual by assessing the presence, absence or development of cancer in the individual comprising:
   i. providing a sample which has been taken from the individual, the sample comprising a population of DNA molecules;
   ii. determining in the population of DNA molecules in the sample the methylation status of a panel of one or more CpGs selected from a panel of CpGs identified at nucleotide positions 61 to 62 in SEQ ID NOs 1 to 29,000;
   iii. deriving a cancer index value based on the methylation status of the one or more CpGs in the panel; and
   iv. assessing the presence, absence or development of cancer in the individual based on the cancer index value, wherein the assay is characterised as having an area under the curve (AUC) of 0.60 or more as determined by receiver operating characteristics (ROC);
b. administering one or more therapeutic or preventative treatments to the individual based on the assessment,
preferably wherein the cancer in breast and/or ovarian cancer, most preferably wherein the cancer is breast cancer.

In any of the methods of treatment encompassed by the invention, the step of predicting the presence or development of cancer, preferably wherein the cancer in breast and/or ovarian cancer, in an individual may involve deriving a cancer index value.

In any of the methods of treatment encompassed by the invention, the step of predicting the presence or development of cancer in an individual may involve the use of any one of the arrays described herein.

In any of the methods of treatment encompassed by the invention, the step of stratifying the individual may involve applying any one of the thresholds according to any one of the assays of the invention described herein.

The step of administering one or more treatments may comprise different treatment steps depending on the stratification of an individual on the basis of their risk of having cancer or on the basis of risk of cancer development, particularly breast and/or ovarian cancer. Particularly the amount of an invasiveness of the treatments administered may vary dependent on the stratification of an individual on the basis of their risk of having cancer or on the basis of their risk of cancer development. The treatments administered to the individual may comprise any treatments considered suitable by a person skilled in the art.

For example, wherein the individual is assessed as not having cancer or as having a low risk of cancer development, and wherein when the cancer index value is about −0.580 or more and less than about −0.280, and preferably wherein the assay comprises determining methylation β-values for each CpG in the panel of one or more CpGs, the individual is subjected to one or more treatments according to their cancer index value, the one or more treatments may comprise intensified screening, preferably wherein the intensified screening comprises any of:
a. a test for a BRCA1 and/or BRCA2 germline mutation;
b. a repeat assay according to any one of the assays of the invention, preferably wherein the repeat assay is performed about two years after the previous assay;
   preferably wherein the cancer in breast and/or ovarian cancer, most preferably wherein the cancer is breast cancer.

Wherein the individual is assessed as having a low risk of having cancer or a low risk of cancer development, and the intensified screening comprising a test for a BRCA1 and/or BRCA2 germline mutation indicates the individual is positive for a BRCA1 and/or BRCA2 germline mutation, the intensified screening further comprises a mammogram. The mammogram is preferably repeated about at least once every year following the test for BRCA1 and/or BRCA2 germline mutation.

Wherein the individual is assessed as having a low risk of having cancer or a low risk of cancer development, and the intensified screening comprising a test for a BRCA1 and/or BRCA2 germline mutation indicates the individual is negative for a BRCA1 and/or BRCA2 germline mutation, the intensified screening may further comprise routine mammography screening. The routine screening preferably comprises a mammogram about every three years following the test for BRCA1 and/or BRCA2 germline mutation.

Wherein the individual is assessed as having a moderate risk of having cancer or as having a moderate risk of cancer development, and wherein when the cancer index value is about −0.280 or more and less than about 0.070, and preferably wherein the assay comprises determining methylation β-values for each CpG in the panel of one or more CpGs, the individual is subjected to one or more treatments according to their cancer index value, the one or more treatments may comprise any of:
a. intensified screening, preferably wherein the intensified screening comprises one or more of:
   i. a test for a BRCA1 and/or BRCA2 germline mutation;
   ii. a breast MRI scan, preferably wherein the scan is repeated about two years after the previous scan;
   iii. a repeat assay according to the invention described herein, preferably wherein the repeat assay is performed about one year after the previous assay;
b. administration of one or more of Denosumab, “selective estrogen receptor modulators” (SERMs), and “selective progesterone receptor modulators” (SPRMs);
   preferably wherein the cancer in breast and/or ovarian cancer, most preferably wherein the cancer is breast cancer.

Wherein the individual is assessed as having cancer or as having a high risk of cancer development, and wherein when the cancer index value is about 0.070 or more, and preferably wherein the assay comprises determining methylation β-values for each CpG in the panel of one or more CpGs, and the individual is subjected to one or more treatments according to their cancer index value, the one or more treatments may comprise any of:
a. intensified screening, preferably wherein the intensified screening comprises one or more of:
   i. a test for a BRCA1 and/or BRCA2 germline mutation;
   ii. a breast MRI scan, preferably wherein the scan is repeated about a year after the previous scan;
   iii. a mammogram, preferably wherein the mammogram is repeated about a year after the previous mammogram;
   iv. a test for CA125, preferably wherein the test is repeated three-monthly;
   v. a test for cell-free tumour DNA methylation in plasma/serum, preferably wherein the test is repeated annually;
   vi. a test for cell-free tumour DNA methylation in vaginal fluid, preferably wherein the test is repeated annually;
   vii. a repeat assay according to the invention described herein, preferably wherein the repeat assay is performed about one year after the previous assay;
b. administration of one or more of Denosumab, “selective estrogen receptor modulators” (SERMs), and “selective progesterone receptor modulators” (SPRMs);
c. a bilateral mastectomy; and/or
d. a bilateral salpingo-oophorectomy;
   preferably wherein the cancer in breast and/or ovarian cancer, most preferably wherein the cancer is breast cancer.

In any of the assays described herein, wherein tests are performed as part of intensified screening the tests may be repeated at any suitable interval. A test for CA125 may be performed three-monthly, six-monthly, annually or about once every two, three or four years. A test for cell-free tumour DNA methylation in plasma/serum may be performed three-monthly, six-monthly, annually or about once every two, three or four years. A test for cell-free tumour DNS methylation in vaginal fluid may be performed three-monthly, six-monthly, annually or about once every two, three or four years. A mammogram may be performed three-monthly, six-monthly, annually or about once every two, three or four years.

Wherein the individual is assessed as having a moderate to high risk of having cancer or a moderate to high risk of cancer development, and/or if the individual has a germline tissue mutation of BRCA1 or BRCA2, preferably BRCA1, the one or more treatments may comprise administration of one or more of Denosumab, “selective estrogen receptor modulators” (SERMs), and “selective progesterone receptor modulators” (SPRMs). The SERMs may comprise Anordin, Bazedoxifene, Broparestrol, Broparestrol, Clomifene, Cyclofenil, Lasofoxifene, Ormeloxifene, Ospemifene, Raloxifene, Tamoxifen, preferably wherein the SERMs comprise Tamoxifen, Bazedoxifene and Raloxifene, preferably wherein the SERMs comprise Tamoxifen, Bazedoxifene and Raloxifene. The SPRMs may comprise Mifepristone, Ulipristal, Asoprisnil, Proellex, Onapristone, Asoprisnil and Lonaprisan.

Wherein the individual is stratified as being at risk of having breast cancer or developing breast cancer, the individual may be subjected to further screening methods as described herein. If the further screening methods result in a negative breast cancer diagnosis, one or more preventative therapies may be administered such as routine screening as described herein, and preventative therapies described herein. Particularly, preventative therapies include administration of one or more doses of one or more selective progesterone receptor modulators (SPRMs) and/or RANKL inhibitors, preferably wherein the SPRMs comprise mifepristone.

In any one of the methods of treatment described herein, the method may further comprise genetic and/or expression profiling of any panel of genes known in the art as being associated with breast cancer. For example, the methods described herein may further comprise genetic and/or expression profiling of any one or more of the genes comprised within the MammaPrint™ test (Cardoso et al, N Engl J Med, 2016; 375:717-729). For any panel of genes known in the art as being associated with breast cancer, the skilled person would be aware of what genetic and/or expression profiles would be considered to be abnormal. Furthermore, the skilled person would be aware of treatments in the art that are known to be efficacious with respect to specific abnormalities observed in profiling any panel of genes known in the art as being associated with breast cancer. For example, upon observing one or more mutations in one or both of the BRCA1 and BRCA2 genes the skilled person would consider administering platinum-based treatments to the individual.

Wherein the individual is predicted as not having breast cancer or having a low risk of having breast cancer or of breast cancer development, the individual may be subjected to risk-prevention treatments. Particularly, for example, if the individual has one or more genetic mutations that predispose an individual to an increased risk of developing breast cancer, the individual may be subjected to risk-prevention treatments. Risk-prevention treatments may comprise any suitable treatment. For example, a risk prevention treatment may be administering one or more doses of mifepristone. In any of the methods described herein, the individual may not harbour breast cancer, but may harbour one or more genetic mutations that pre-dispose the individual to breast cancer such as one or more mutations in the BRCA genes. Other mutations may include any mutations in the art that are considered to pre-dispose individuals to breast cancer. In any of the methods of treatment described herein, the individual may not harbour breast cancer but may harbour one or more genetic mutations that pre-dispose the individual to breast cancer, and this individual may be subjected to any of the methods of monitoring described herein. For example, in any of the methods described herein, the individual does not harbour breast cancer and harbours one or more mutations that predispose the individual to an increased risk of developing breast cancer, and wherein one or more treatments administered to the individual comprises one or more doses of mifepristone.

Other exemplary treatments comprise one or more surgical procedures, one or more chemotherapeutic agents, one or more cytotoxic chemotherapeutic agents one or more radiotherapeutic agents, one or more immunotherapeutic agents, one or more biological therapeutics, one or more anti-hormonal treatments or any combination of the above following a positive diagnosis of cancer.

In any of the methods of treatment described herein, the individual may particularly be administered treatments recited in Table 8. Four sub-groups defined by ranges of cancer index values are specified in Table 8 as corresponding to preferred clinical actions, comprising intensified screening, administration of therapeutics and surgery.

Cancer treatments may be administered to an individual harbouring cancer or at risk of cancer development, in an amount sufficient to prevent, treat, cure, alleviate or partially arrest cancer or one or more of its symptoms. Such treatments may result in a decrease in severity, and/or decreased cancer index value, of cancer symptoms, or an increase in frequency or duration of symptom-free periods. A treatment amount adequate to accomplish this is defined as “therapeutically effective amount”. Effective amounts for a given purpose will depend on the severity of cancer and/or the individual's cancer index value as well as the weight and general state of the individual. As used herein, the term “individual” includes any human, preferably the human is a woman. As used herein, “treatment” is to be considered synonymous with “therapeutic agent”.

The following therapeutic agents may be administered to an individual based on their cancer risk alone or in combination with any other treatment described herein. The therapeutic agent may be directly attached, for example by chemical conjugation, to an antibody. Methods of conjugating agents or labels to an antibody are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

Cytotoxic chemotherapeutic agents are well known in the art. Cytotoxic chemotherapeutic agents, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

A cytotoxic chemotherapeutic agent may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Radiotherapeutic agents may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to an antibody, antigen-binding fragment, variant, fusion or derivative thereof in known ways. For example, EDTA or another chelating agent may be attached to the binding moiety and used to attach 111In or 90Y. Tyrosine residues may be directly labelled with 1251 or 1311.

A cytotoxic chemotherapeutic agent may be a suitable indirectly-cytotoxic polypeptide. In a particularly preferred embodiment, the indirectly cytotoxic polypeptide is a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. In a preferred embodiment, the cytotoxic moiety is capable of converting a non-cytotoxic prodrug into a cytotoxic drug.

In any of the methods of treatment described herein, the one or more treatments that the individual is subjected to may be repeated on one or more occasions. The one or more treatments may be repeated at regular intervals. The repetitive nature of the treatment administration may depend on the particular treatment being administered. Some treatments may require repetitive administration at greater frequency than others. The skilled person would be aware of the frequency of administration required for therapies known in the art. The one or more treatments may be repeated weekly, two weekly, three weekly, four weekly, monthly, three monthly, six monthly, yearly, two yearly, three yearly, four yearly, or five yearly.

In any of the methods described herein, when the individual is assessed as having a cancer index value of less than about −0.580, and preferably wherein the assay comprises determining methylation β-values for each CpG in the panel of one or more CpGs, no treatment is administered to the individual.

### Methods of Monitoring

The invention also provides methods of monitoring the risk of the presence or development of cancer in an individual.

“Monitoring” in the context of the present invention may refer to longitudinal assessment of an individual's risk of harbouring cancer or risk of cancer development. This longitudinal assessment may be carried out according to the assays of the invention described herein. This longitudinal assessment may involve performance of the assays of the invention described herein to predict the presence or development of cancer in an individual at more than one time point over the course of an undetermined time window. The time window may be any period of time whilst the individual is still living. The time window may persist for the lifetime of the individual. The time window may persist until the individual's risk of harbouring cancer or risk of cancer development falls below a certain level. The level may be a particular cancer index value.

The invention thus encompasses a method of monitoring for the presence, absence or development of cancer, particularly breast and/or ovarian cancer, most preferably breast cancer, in an individual, the method comprising:
a. assessing the presence or absence of cancer in an individual or assessing cancer development in an individual to establish a cancer status for the individual by performing any one of the assays of the invention described herein at a first time point;
b. assessing the presence or absence of cancer in the individual or assessing cancer development in the individual to establish a cancer status for the individual by performing any one of the assays of the invention described herein at one or more further time points, preferably wherein the cancer status of the individual in steps a and b are assessed using the same assay; and
c. monitoring any change in the cancer status of the individual between time points.

The invention also encompasses a method of monitoring for the presence, absence or development of cancer, particularly breast and/or ovarian cancer, most preferably breast cancer, in an individual, the method comprising:
a. assessing the presence or absence of cancer in an individual or assessing cancer development in an individual to establish a cancer status for the individual by performing an assay at a first time point, comprising:
   i. providing a sample which has been taken from the individual, the sample comprising a population of DNA molecules;
   ii. determining in the population of DNA molecules in the sample the methylation status of a panel of one or more CpGs selected from a panel of CpGs identified at nucleotide positions 61 to 62 in SEQ ID NOs 1 to 29,000;
   iii. deriving a cancer index value based on the methylation status of the one or more CpGs in the panel; and
   iv. assessing the presence, absence or development of cancer in the individual based on the cancer index value, wherein the assay is characterised as having an area under the curve (AUC) of 0.60 or more as determined by receiver operating characteristics (ROC);
   b. assessing the presence or absence of cancer in the individual or assessing cancer development in the individual to establish a cancer status for the individual by performing the assay of steps a(i) to a(iv) or by performing any one of the assays of the invention described herein at one or more further time points; and
   c. monitoring any change in the cancer status of the individual between time points.

In any of the methods of monitoring described herein, the steps of assessing the presence, absence or development of cancer in an individual based on a cancer index value may involve the application of threshold values. Threshold values can provide an indication of an individual's risk of having cancer or an individual's risk of cancer development. For example, cancer index values may indicate a high or low risk of harbouring or developing cancer. In any of the methods of monitoring encompassed by the invention, the step of predicting the presence, absence or development of cancer in an individual involves deriving a cancer index value.

The invention further encompasses a method of measuring methylation in a patient at multiple time points comprising (a) assessing the presence, absence or development of cancer in an individual by performing any one of the assays of the invention described herein at a first time point; (b) assessing the presence, absence or development of cancer in the individual by performing any one of the assays of the invention described herein at one or more further time points, and (c) detecting differential methylation status between (a) and (b).

In any of the methods of monitoring described herein, the individual may already harbour cancer, particularly breast and/or ovarian cancer. The individual may not have cancer. The individual may not harbour cancer. The individual may not harbour cancer but may harbour one or more genetic mutations that predispose the individual to an increased risk of cancer development e.g. the individual may harbour one or more mutations in a BRCA gene. Other mutations may include any mutations in the art that are considered to pre-dispose individuals to cancer. In any of the methods of monitoring described herein, the individual may not harbour cancer but may harbour one or more genetic mutations that pre-dispose the individual to cancer, and this individual may be subjected to any of the methods of monitoring described herein in order to determine their risk of having cancer or of developing cancer. For example, in any of the methods described herein, the individual does not harbour cancer and harbours one or more mutations that predispose the individual to an increased risk of developing cancer, particularly breast and/or ovarian cancer, and wherein one or more treatments are administered to the individual in accordance with any of the methods of treatment described herein as a method of prophylaxis. In any of the methods described herein, the individual does not harbour cancer and harbours one or more mutations that predispose the individual to an increased risk of developing cancer, and wherein one or more treatments are administered to the individual in accordance with any of the methods of treatment described herein as a method of prophylaxis, and wherein the one or more treatments administered to the individual comprises one or more doses of Denosumab, “selective estrogen receptor modulators” (SERMs), and “selective progesterone receptor modulators” (SPRMs). The SERMs may comprise Anordin, Bazedoxifene, Broparestrol, Broparestrol, Clomifene, Cyclofenil, Lasofoxifene, Ormeloxifene, Ospemifene, Raloxifene, Tamoxifen. The SPRMs may comprise Mifepristone, Ulipristal, Asoprisnil, Proellex, Onapristone, Asoprisnil and Lonaprisan. Preferably the one or more prophylactic treatments administered to the individual comprises Tamoxifen, Bazedoxifene and Raloxifene.

In any of the methods of monitoring described herein, depending on the risk of the presence or development of cancer in the individual, one or more treatments are administered to the individual according to any one of the methods of treatment encompassed by the invention and described herein, or wherein when the cancer index value of the individual is less than about −0.580 no treatment is administered to the individual. Different treatments may be administered depending on the stratification of an individual on the basis of their risk of harbouring cancer or on the basis of their risk of cancer development. The method may further comprise administration of one or more treatments according to the methods of treatment described herein.

The cancer index value may change between any two or more time points. For this reason, longitudinal monitoring of an individual's cancer index value could be of particular benefit to the assessment of, for example, cancer progression, prevention of recurrence of cancer, cancer treatment efficacy, or cancer efficacy.

In any of the methods of monitoring described herein, the one or more further time points may be any suitable time point. Preferably the one or more further time points may of suitable distance apart for sufficiently frequent screening in order to predict any particularly early onset cases of presence or development of cancer in an individual. Preferably the one or more further time points may be of suitable distance apart for assessing the efficacy of one or more treatments. Preferably the one or more further time points may be of suitable distance apart for predicting whether an individual remains free of cancer after a successful course of treatment. The one or more further time points may be about monthly, about two monthly, about three monthly, about four monthly, about five monthly, about six monthly, about seven monthly, about eight monthly, about nine monthly, about ten monthly, about eleven monthly, about yearly, about two yearly, or more than two yearly.

In any of the methods of monitoring described herein, changes may be made to the one or more treatments wherein a positive or negative responses to the one or more treatments are observed. Treatments may be changed in accordance with the methods of treatments described herein. Treatments may particularly be changed if the individual's risk stratification, based on their cancer index value, changes.

In any of the methods of monitoring encompassed by the invention, the step of predicting the presence or development of cancer in an individual may involve the use of any one of the arrays described herein.

### Biological Samples

The assays described herein are preferably performed on samples comprising epithelial cells, particularly obtained from an anatomical site other than the ovary or endometrium. The sample may particularly be derived from the cervix, the vagina, the buccal area, blood and/or urine. The sample is preferably a cervical liquid-based cytology sample, and more preferably a cervical smear sample.

Preferably, any one of the assays described herein for assessing the presence, absence or development of cancer in an individual comprises providing a sample which has been taken from the individual. Preferably the individual is a woman.

In any of the assays described herein, the assay may or may not encompass the step of obtaining the sample from the individual. In assays which do not encompass the step of obtaining the sample from the individual, a sample which has previously been obtained from the individual is provided.

The sample may be provided directly from the individual for analysis or may be derived from stored material, e.g. frozen, preserved, fixed or cryopreserved material.

In any of the assays described herein, the sample may be self-collected or collected by any suitable medical professional.

Any of the assays described herein, the sample may comprise cells. The sample may comprise genetic material such as DNA and/or RNA.

Any of the assays described herein may involve providing a biological sample from the patient as the source of patient DNA for methylation analysis.

Any of the assays described herein may involve obtaining patient DNA from a biological sample which has previously been obtained from the patient.

Any of the assays described herein may involve obtaining a biological sample from the patient as the source of patient DNA for methylation analysis. The sample may be self-collected or collected by any suitable medical professional. Procedures for obtaining a biological sample include biopsy.

Methods for sample isolation and for the subsequent extraction and isolation of DNA from such cell or tissue samples in preparation for assessing DNA methylation, are well known to those skilled in the art. In the context of the assays or methods described herein, the entirety of a sample may be used, or alternatively cells may be concentrated or cell types may be fractionated in order to only apply subsets of one or more cell types to the present assays or methods. Any suitable methods of concentration or fractionation may be used.

In any one of the assays described and defined herein the sample from the individual, or the sample which has been taken from the individual, may derive from a tissue which is different from the tissue which harbours the tumour, if a tumour is present in the individual. Accordingly, in any one of the assays described and defined herein the sample from the individual, or the sample which has been taken from the individual, may not comprise nucleic acid, including DNA, which derives from the tumour, i.e. tumour-specific nucleic acid, including tumour-specific DNA. Thus, consistent with the data set out in the examples and the disclosures herein, methylation profiles derived from DNA molecules in the sample are used as surrogate markers for tumour-specific nucleic acid, including tumour-specific DNA, which exists at an anatomical site in the body of the individual which is remote from the anatomical site from which the sample is derived. A skilled person would able to identify the absence of tumour-specific DNA in any given population of sample-specific DNA molecules by routine means, such as determining the absence of known genetic mutations which are characteristic of the particular cancer, by e.g. sequence based screening. However, the performance of any one of the assays described and defined herein does not require any assessment to verify the absence of tumour-specific DNA in any given population of DNA molecules.

### Types of Cancers

The methods described herein may be applied to any cancer. Preferably, the methods described herein may be applied to breast cancer and/or ovarian cancer. The methods described herein are most preferably applied to breast cancer.

The cancer may be a primary cancer lesion. The cancer may be a secondary cancer lesion. The cancer may be a metastatic lesion.

In assays described herein, wherein the assays is for assessing the presence, absence or development of breast cancer, the breast cancer may be a ductal carcinoma in situ or an invasive ductal carcinoma such as tubular type invasive ductal carcinoma (IDC), medullary type IDC, mucinous type IDC, papillary type IDC or cribriform type IDC.

The breast cancer may be an invasive carcinoma such as a pleomorphic carcinoma, carcinoma with osteoclast giant cells, carcinoma with choriocarcinoma features, carcinoma with melanotic features. The invasive breast carcinoma may be an invasive lobular carcinoma, tubular carcinoma, invasive cribriform carcinoma, medullary carcinoma, mucinous carcinoma and other tumours with abundant mucin such as mucinous carcinoma, cystadenocarcinoma and column a cell mucinous carcinoma, signet ring cell carcinoma. The invasive breast carcinoma may be a neuroendocrine tumour such as solid neuroendocrine carcinoma (carcinoid of the breast), atypical acarcinoid tumour, small cell/oat cell carcinoma, large cell neuroendocrine carcinoma. The invasive breast carcinoma may be an invasive papillary carcinoma, invasive micropapillary carcinoma, apocrine carcinoma, metaplastic carcinomas such as pure epithelial metaplastic carcinomas including squamous cell carcinoma, adenocarcinoma with spindle cell metaplasia, adenosquamous carcinoma, mucoepidermoid carcinoma, mixed epithelial/mesenchymal metaplastic carcinomas, matrix-producing carcinoma, spindle cell carcinoma, carcinosarcoma, squamous cell carcinoma of mammary origin, metaplastic carcinoma with osteoclastic giant cells. The invasive breast carcinoma may be a lipid-rich carcinoma, secretory carcinoma, oncocytic carcinoma, adenoid cystic carcinoma, acinic cell carcinoma, glycogen-rich clear cell carcinoma, sebaceous carcinoma, inflammatory carcinoma, bilateral breast carcinoma.

The breast cancer may be a mesenchymal breast tumour. The mesenchymal tumour may include sarcoma. The mesenchymal breast tumour may be a hemangioma, angiomatosis, Hemangiopericytoma, Pseudoangiomatous stromal hyperplasia, Myofibroblastoma, Fibromatosis (aggressive), Inflammatory myofibroblastic tumor, Lipoma Angiolipoma, Granular cell tumour, Neurofibroma, Schwannoma, Angiosarcoma, Liposarcoma, Rhabdomyosarcoma, Osteosarcoma, Leiomyoma, Leiomyosarcoma.

The breast cancer may be a malignant lymphoma such as Non-Hodgkin lymphoma.

The breast cancer may be a metastatic tumour in which the primary lesion originated in a tissue other than the breast.

The breast cancer may be a precursor breast cancer lesion. The precursor breast cancer lesion may be a Lobular neoplasia, lobular carcinoma in situ, Intraductal proliferative lesions, Usual ductal hyperplasia, Flat epithelial hyperplasia, Atypical ductal hyperplasia, Ductal carcinoma in situ, Microinvasive carcinoma, Intraductal papillary neoplasms, Central papilloma, Peripheral papilloma, Atypical papilloma, Intraductal papillary carcinoma, Intracystic papillary carcinoma.

The breast cancer may be a myoepithelial breast cancer lesion. The myoepithelial breast cancer lesion be myoepitheliosis, adenomyoepithelial adenosis, adenomyoepithelioma, malignant myoepithelioma.

The breast cancer may be a fibroepithelial breast tumour. The fibroepithelial breast tumour may be a fibroadenoma, phyllodes tumour, periductal stromal sarcoma, mammary hamartoma.

The breast cancer may be Paget's disease of the nipple.

Any of the assays described herein may additionally, or alternatively, be for assessing the presence, absence or development of ovarian cancer.

In assays described herein, wherein the assays is for assessing the presence, absence or development of ovarian cancer, the ovarian cancer may preferably be serious carcinoma, mucinous carcinoma, endometrioid carcinoma, clear cell carcinoma, lop malignant potential (LMP) tumor, borderline epithelial ovarian cancer, teratoma, dysgerminoma, endodermal sinus tumor, Choriocarcinoma, granulosa-theca tumor, Sertoli-Leydig tumor, granulosa cell tumor, small cell carcinoma of the ovary or primary peritoneal carcinoma.

### Arrays and Kits

The invention also encompasses arrays capable of discriminating between methylated and non-methylated forms of CpGs as defined herein; the arrays may comprise oligonucleotide probes specific for methylated forms of CpGs as defined herein and oligonucleotide probes specific for non-methylated forms of CpGs as defined herein.

In any of the arrays described herein, the array may comprise oligonucleotide probes specific for a methylated form of each CpG in a CpG panel and oligonucleotide probes specific for a non-methylated form of each CpG in the panel; wherein the panel consists of at least 500 CpGs selected from the CpGs identified at nucleotide position 61 to 62 in SEQ ID NOs 1 to 29,000.

The panel may consist of at least 1000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 1000.

The panel may consist of at least 2000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 2000.

The panel may consist of at least 3000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 3000.

The panel may consist of at least 4000 CpGs selected from the CpGs identified at nucleotide position 61 to 62 in SEQ ID NOs 1 to 29000, preferably wherein the CpGs are the CpGs identified at nucleotide position 61 to 62 in SEQ ID NOs 1 to 4000.

The panel may consist of at least 5000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 5000.

The panel may consist of at least 6000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 6000.

The panel may consist of at least 7000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 7000.

The panel may consist of at least 8000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 8000.

The panel may consist of at least 9000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 9000.

The panel may consist of at least 10000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 10000.

The panel may consist of at least 11000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 11000.

The panel may consist of at least 12000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 12000.

The panel may consist of at least 13000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 13000.

The panel may consist of at least 14000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 14000.

The panel may consist of at least 15000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 15000.

The panel may consist of at least 16000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 16000.

The panel may consist of at least 17000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 17000.

The panel may consist of at least 18000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 18000.

The panel may consist of at least 19000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 19000.

The panel may consist of at least 20000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 20000.

The panel may consist of at least 21000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 21000.

The panel may consist of at least 22000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 22000.

The panel may consist of at least 23000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 23000.

The panel may consist of at least 24000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 24000.

The panel may consist of at least 25000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 25000.

The panel may consist of at least 26000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 26000.

The panel may consist of at least 27000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 27000.

The panel may consist of at least 28000 CpGs selected from the CpGs identified at nucleotide position 61 to 62 in SEQ ID NOs 1 to 29,000, preferably wherein the CpGs are the CpGs identified at nucleotide position 61 to 62 in SEQ ID NOs 1 to 28000.

The panel may consist of all CpGs identified in SEQ ID NOs 1 to 29,000.

In some embodiments the array is not an Infinium MethylationEPIC BeadChip array or an Illumina Infinium HumanMethylation450 BeadChip array.

Separately or additionally, in some embodiments the number of CpG-specific oligonucleotide probes of the array is 482,000 or less, 480,000 or less, 450,000 or less, 440,000 or less, 430,000 or less, 420,000 or less, 410,000 or less, or 400,000 or less, 375,000 or less, 350,000 or less, 325,000 or less, 300,000 or less, 275,000 or less, 250,000 or less, 225,000 or less, 200,000 or less, 175,000 or less, 150,000 or less, 125,000 or less, 100,000 or less, 75,000 or less, 50,000 or less, 45,000 or less, 40,000 or less, 35,000 or less, 30,000 or less, 25,000 or less, 20,000 or less, 15,000 or less, 10,000 or less, 5,000 or less, 4,000 or less, 3,000 or less or 2,000 or less.

The CpG panel may comprise any set of CpGs defined in the assays of the invention described herein.

The arrays of the invention may comprise one or more oligonucleotides comprising any set of CpGs defined in the assays of the invention, wherein the one or more oligonucleotides are hybridized to corresponding oligonucleotide probes of the array.

The invention also encompasses a process for making a hybridized array described herein, comprising contacting an array according to the present invention with a group of oligonucleotides comprising any set of CpGs defined in the assays of the invention.

Any of the arrays as defined herein may be comprised in a kit. The kit may comprise any array as defined herein together with instructions for use.

The invention further encompasses the use of any of the arrays as defined herein in any of the assays for determining the methylation status of CpGs for the purposes of predicting the presence or development of cancer in an individual.

The following Examples serve to illustrate but not to limit the invention.

### EXAMPLES

In the Examples described herein, WID-BC-Index is a cancer index value wherein the index value has been determined by assaying in a population of DNA molecules derived from a given sample from an individual the methylation status of a panel of CpGs selected from the CpGs defined by SEQ ID NOs: 1 to 29,000.

In some instances within the Examples, all CpGs defined by SEQ ID NOs: 1 to 29,000 have been included in the panel which has been assayed to obtain a cancer index value. In addition, specific sub-selections of CpGs from among the 29,000 CpGs defined by SEQ ID NOs: 1 to 29,000 have been included in the panel which has been assayed to obtain a cancer index value. In these instances, the cancer index value's ability to discriminate between cancer positive and cancer negative women is described, wherein discriminatory ability of the index is characterised by AUC and received operating characteristics.

To date, DNA methylation of only a very few genes has been assessed in cervical smear samples from women with and without ovarian cancer and not one of these very small studies reached significance after multiple test adjustment.

Here, the inventors performed an epigenome-wide DNAme analysis in cervical smear samples from women who had recently been diagnosed with breast cancer, and in matched controls, and established the WID-BC-index (Women's risk IDentification for Breast Cancer index) which the inventors validated in buccal samples and in an independent set of cervical samples. Further validation was done in samples from women with ovarian cancer and a set of matched cervical, buccal, and blood samples from BRCA mutation carriers. In addition, the inventors assessed the WID-BC-index in a larger number of different cell types as well as in normal breast and breast cancer tissue samples.

### Materials and Methods

### Study Design and Epidemiological Data Acquisition

The study was conducted as part of a multi-centre study involving several recruitment sites in 5 European countries (i.e. the UK, Czech Republic, Italy, Norway and Germany). Participants were aged ≥18 years. Prior to taking part, each prospective study volunteer was given a Participant Information Sheet as well as a Consent Form and the rationale for the study was explained. Additional resources, including an explanatory video and further online resources, were also made available. Women diagnosed with breast or ovarian cancer (case) or a non-malignant benign gynaecological condition (control) were approached during outpatient hospital clinics, while women recruited as BRCA mutation carriers or healthy volunteers from the general population (control) were approached via outreach campaigns, public engagement, and as part of cervical screening programmes. After signing an informed consent, participants completed an epidemiological questionnaire as well as a feedback form after their participation. The study itself is a sub-study of the FORECEE (4C) Programme, which has ethical approval from the UK Health Research Authority (REC 14/LO/1633) and other contributing centres.

The epidemiological survey was administered via the Qualtrics application on dedicated iPads. The survey contained questions relating to health habits, relevant risk factors, and also made enquiries as to historical health habits, as well as obtaining a thorough medical and obstetric history. Cervical samples were collected at appropriate clinical venues by trained staff and the cervical smears were carried out by a small group of research midwives or physicians with a view to establishing standard practice. Buccal samples were collected using Copan 4N6FLOQ Swabs, Thermofisher Scientific.

Biological samples were given an anonymous Participant ID Number which was assigned to the person's name in a securely stored link file. Following sample taking, an email survey was sent to each participant, enabling them to feedback with respect to the recruitment process. Women with a current diagnosis of (a) primary breast cancer with poor prognosis features (Grade III and/or T2/3 and/or N1/2 and/or HR-ve) or (b) malignant invasive epithelial ovarian cancer and recruited prior to receiving any systemic treatment (chemo- or antihormonal or Herceptin, etc.) or surgery or radiotherapy were eligible as breast, ovarian or endometrial cancer cases respectively. For the FORECEE Discovery set controls were initially matched one-to-one with cases based on menopausal status, age (5 year age ranges where possible), and recruitment centre/country. However, due to an imbalance in recruitment of cases and controls at some centres, a number of cases were matched on age and menopausal status alone. Cancer histological data was collected post-recruitment either by clinicians directly involved in the diagnosis/treatment of the cancer cases or by a nominated data manager with access to the in-house hospital systems.

### Cervical Smear Sample Collection

Cervical smears were taken at collaborating hospitals and recruitment centres using the ThinPrep system (Hologic Inc., cat #70098-002). Cervical cells were sampled from the cervix using a cervix brush (Rovers Medical Devices, cat #70671-001) which was rotated 5 times through 360 degrees whilst in contact with the cervix to maximise cell sampling. The brush was removed from the vagina and immersed in a ThinPrep vial containing Preserve-cyt fluid and then pushed against the bottom of the vial 10 times to facilitate release of the cells from the brush into the solution. The sample vial was sealed and stored locally at room temperature. Buccal cells were collected using two Copan 4N6FLOQ Buccal Swabs (Copan Medical Diagnostics, cat #4504C) by firmly brushing the swab head 5-6 times against the buccal mucosa of each cheek. The swabs were re-capped and left to dry out at room temperature within the sampling tube which contains a drying desiccant. 2.5 ml of venous whole blood was collected in PAX gene blood DNA tubes (BD Biosciences #761165) and stored locally at 4° C. All samples were shipped to UCL at ambient temperature.

### Breast Tissue Samples

The inventors have analysed two independent sets of breast tissue samples. The first set contained a total of 56 breast samples from premenopausal women aged 19-54 years: normal breast tissue from 14 women who underwent cosmetic breast operations, normal breast tissue from women who underwent prophylactic mastectomies due to a BRCA1 (n=9) or a BRCA2 (n=5) mutation, and 14 women who had breast surgery due to a triple negative breast cancer and who provided both normal adjacent breast tissue as well as cancer tissue samples. All samples were collected fresh from theatre and samples processed within 1 hr of surgical excision. Fresh samples were frozen rapidly in Liquid Nitrogen and stored at −80° C. Ethical approval was obtained from the NRES Committee East of England (reference number 15/EE/0192).

The second set of samples were obtained from the clinical trial “The Effect of a Progesterone Receptor Modulator on Breast Tissue in Women With BRCA-1 and -2 Mutations—a Placebo Controlled RCT” (ClinicalTrials.gov Identifier: NCT01898312; regional ethical review board at Karolinska Institutet permit 2009/144-31/4). Study subjects were healthy premenopausal women aged 18-43 years with regular menstrual cycles lasting 25-35 days and with no contraindications to mifepristone. The main exclusion criteria were: use of any hormonal or intrauterine contraception and pregnancy or breastfeeding 2 months prior to the study; a history of breast cancer or other malignancies and adnexal abnormality upon transvaginal ultrasound examination. All women were instructed to use barrier contraceptive methods throughout the duration of the study.

After signing an informed consent, study subjects were randomised into two groups. One group (i.e. 11 BRCA carriers and 9 controls) was treated with 50 mg mifepristone (one quarter of 200 mg Mifegyne®, Exelgyn, Paris, France) every other day for two months (56 days) starting on the first day of the menstrual cycle. As mifepri stone is only available in 200 mg tablets in Sweden, a study nurse divided the tablets into 4 parts and instructed the study subjects to take one part every other day. The placebo group (i.e. 4 BRCA carriers and 11 controls) received B-vitamin tablets which are visually identical (one quarter of TrioBe® Recip) to mifepristone. Tablets were dispensed for two weeks at a time.

Core needle aspiration biopsies were collected during the luteal phase. The biopsies were collected under ultrasound guidance from the upper outer quadrant of one breast using a 14 Gauge needle with an outer diameter of 2.2 mm. The end-of-treatment breast biopsy was taken from the same area.

### Sample Processing and DNA Extraction

When preparing for sample storage in the laboratory, cervical smear samples were poured into 50 ml Falcon tubes and left to sediment at room temperature for 2 hours. 1 mL wide bore tips were then used to transfer the enriched cellular sediment into a 2 mL vial. The cervical sediments were washed twice with PBS, lysed, and stored temporarily at −20° C. ahead of extraction. The Copan 4N6FLOQ Buccal Swabs were cut and lysed sequentially in the same aliquot of lysis buffer prior to temporary storage at −20° C. ahead of extraction. Whole blood samples were simply held transiently at −20° C. until DNA extraction. DNA was extracted from whole blood, cervical and buccal tissue lysates on a Hamilton Star liquid handling platform using the Nucleo-Mag Blood 200 ul kit (Macherey Nagel, cat #744501.4) with prior modifications for optimal lysis of cervical cell pellets and paired buccal swabs. For breast tissues, DNA was extracted from up to 40 mg of tissue using the Lipid Tissue kit from Macherey Nagel (cat #740471.50), and the manufacturer's instructions were followed. DNA concentration and quality absorbance ratios were measured using Nanodrop-8000, Thermoscientific Inc. Extracted DNA was stored at −80° C. until further analysis.

### DNA Methylation Array Analysis

Cervical, buccal and breast tissue DNA was normalised to 25 ng/ul and 500 ng total DNA was bisulfite modified using the EZ-96 DNA Methylation-Lightning kit (Zymo Research Corp, cat #D5047) on the Hamilton Star Liquid handling platform. 8 ul of modified DNA was subjected to methylation analysis on the Illumina InfiniumMethylation EPIC BeadChip (Illumina, CA, USA) at UCL Genomics according to the manufacturer's standard protocol.

### Microbiome Analysis

The inventors have described the microbiome analyses recently 26. In brief, during wet laboratory processing, each cervical smear sample was poured into 50 mL Falcon tubes (Fisher Scientific, Loughborough, UK) and left to sediment at room temperature for 2 h. 1 mL wide-bore tips were used to transfer cervical cell enriched sediment to 2 mL cryovials, which were stored at −80° C. Next-generation sequencing was done on all samples in both sets. Total DNA extraction from cervical smears was done with the QIAsymphony DSP Virus/Pathogen kit (Qiagen, Hilden, Germany) according to the manufacturer's instructions. Sequencing and taxonomical classification of bacterial species in cervical samples was done by Eurofins Genomics Europe Sequencing (Constance, Germany) as previously described.12 In brief, the hypervariable V1-V3 region of the bacterial small subunit ribosomal DNA (16S rRNA genes) was amplified with primers modified from work by Klindworth and colleagues (27F 5′-AGAGTTTGATCCTGGCTCAG and 534R 5′-ATTACCGCGGCTGCTGG) to decrease melting temperature stringency and, as a result, increase taxonomic coverage, including Illumina adapters and dual index barcodes. These barcoded libraries were equimolarly pooled and sequenced on the HiSeq2500 with 300 bp paired-end reads (HiSeq Rapid SBS Kit v2; Illumina, San Diego, CA, USA). The 16S rRNA gene PCRs, next-generation sequencing libraries, and pooled libraries were quality checked by Eurofins Genomics via electropherograms and fluorometer concentration determinations. Additionally, positive and negative controls were included in each library preparation, including DNA extraction batches.

To process the 16S rRNA gene-sequencing data, the demultiplexed sequencing reads were quality checked, trimmed, and filtered with Sickle (version 1.33; settings: −q 20, −1 246, −n yes), and adapters and primers were removed with Cutadapt (version 1.10; settings: −minimum-length 246). Overlapping paired-end reads were merged for full-length V1-V3 16S amplicons with FLASH (version 1.2.11; settings: −min-overlap 10, −max-overlap 300, −max-mismatch-density 0.25) and clustered with CD-HIT (version 4.6; settings: −c 0.99 and cluster filtered, resulting in a minimum number of two members), and then chimeric sequences were removed with UCHIME (version 4.2.40). Operational taxonomic units were assigned with BLASTN+(version 2.4.0; −evalue 1e-06) via a non-redundant 16S rRNA reference database from the Ribosomal Database Project (release 11), and filtered to ensure that only high-quality operational taxonomic units were included (ie, 97% identity threshold, 95% alignment coverage, 376 query length threshold, and 10% bitscore threshold). Taxonomic classification was based on the US National Center for Biotechnology Information taxonomy. To establish species diversity and evenness, the inventors calculated both the Shannon and the Simpson indices for each sample.

For the purposes of this study, the inventors collapsed Ravel's 29 four Lactobacillus community groups I, II, III, and V into one microbial community, which the inventors referred to as community type L. The inventors divided samples into people whose cervicovaginal microbiota consisted of at least 50% community type L and those whose microbiota consisted of less than 50% community type L, which the inventors referred to as community type 0. Generally, community type L microbiota are dominated by a Lactobacillus species, whereas type 0 microbiota communities have more bacterial diversity, are composed of typical obligate and facultative anaerobe genera (such as Gardnerella or Atopobium species), and are in turn associated with aerobic vaginitis and bacterial vaginosis.

### Methylation Analysis

All methylation microarray data were processed through the same standardised pipeline. Raw data was loaded using the R package minfi. Any samples with median methylated and unmethylated intensities <9.5 were removed. Any probes with a detection p-value >0.01 were regarded as failed. Any samples with >10% failed probes, and any probes with >10% failure rate were removed from the dataset. Beta values from failed probes (approximately 0.001% of the dataset) were imputed using the impute.knn function as part of the impute R package.

Non-CpG probes (2,932), SNP-related probes as identified by Zhou et. al. 30 (82,108), and chrY probes were removed from the dataset. An additional 6,102 previously identified probes that followed a trimodal methylation pattern characteristic of an underlying SNP were removed.

Background intensity correction and dye bias correction was performed using the minfi single sample preprocessNoob function. Probe bias correction was performed using the beta mixture quantile normalisation (BMIQ) algorithm.

The fraction of immune cell contamination, and the relative proportions of different immune cell subtypes in each sample, were estimated using the EpiDISH algorithm using the epithelial, fibroblast and immune cell reference dataset. The top 1,000 most variable probes (ranked by standard deviation) were used in a principal component analysis. Statistical tests were performed in order to identify any anomalous associations between plate, sentrix position, date of array processing, date of DNA creation, study centre, immune contamination fraction, age, type (case versus control) and the top ten principal components. Finally, two-thirds of the discovery dataset was randomly selected for use as the training dataset and the remaining third was allocated to the internal validation dataset. This split was carried out once, and the same training and validation sets were used in all subsequent analyses.

113 samples were downloaded from the ENCODE database (https://www.encodeproject.orgi; see Additional Data). The beta mixture quantile normalisation was applied to these samples after using minfi to extract beta values.

### Statistical Analyses for Classifier Development

Contamination by immune cells presented a challenge with respect to the identification of differentially methylated positions (DMPs) as differential methylation that occurred solely in epithelial cells was diminished in samples with high IC and vice versa. In order to overcome this, the inventors linearly regressed the beta values on IC for each CpG site, the linear models being fitted to cases and controls separately. The intercept points at IC=0 were used as estimates of mean beta values in cases and controls in a pure epithelial cell population. The difference between these intercept points provided a delta-beta estimate in epithelial cells. The difference between intercept points at IC=1 provided immune cell delta-beta estimates. A list of ranked CpGs was produced according to delta-beta estimates in epithelial cells.

The R package glmnet was used to train classifiers with a mixing parameter value of alpha=0 (ridge penalty) and alpha=1 (lasso penalty) with binomial response type. Data from the training dataset were used to fit the classifiers. A ranked list of CpGs was generated by taking the CpG with the largest epithelial delta-beta, followed by the CpG with the largest immune delta-beta, followed by the next largest epithelial delta-beta and so forth (any duplicates were removed). The top n CpGs from the list of ranked CpGs were used as inputs to the classifier. Ten-fold cross-validation was used inside the training set by the cv.glmnet function in order to determine the optimal value of the regularisation parameter lambda. The AUC was used as a metric of classifier performance which was evaluated on the internal validation dataset as a function of n, the number of CpGs used as inputs during training. The maximum value of n was 30,000.

The optimal classifier was selected based on the highest AUC obtained in the internal validation dataset. Once the optimal number of inputs was determined, the training and internal validation datasets were combined and the classifier was refitted using the entire discovery dataset with alpha and lambda fixed to their optimal values. This finalised classifier was then applied to the external validation dataset and the corresponding AUC was computed.

Denoting the top n CpGs as x₁, . . . , xₙ and the regression coefficients from the trained classifier as w₁, . . . , wₙ then WID-BC-index=Σᵢ₌₁ⁿ(wᵢxᵢ−μ)/σ where μ and σ are defined as the mean and standard deviation of the quantity Σᵢ₌₁ⁿwᵢxᵢ in the training dataset (that is, the index is scaled to have zero mean and unit standard deviation in the training dataset).

### Decomposition of Index

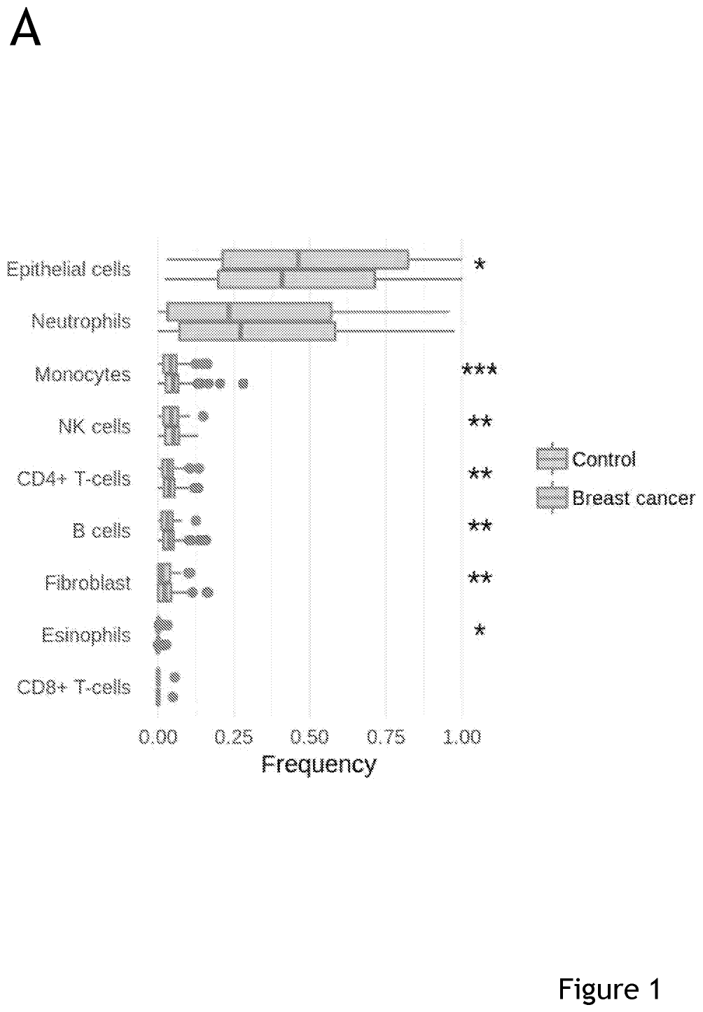

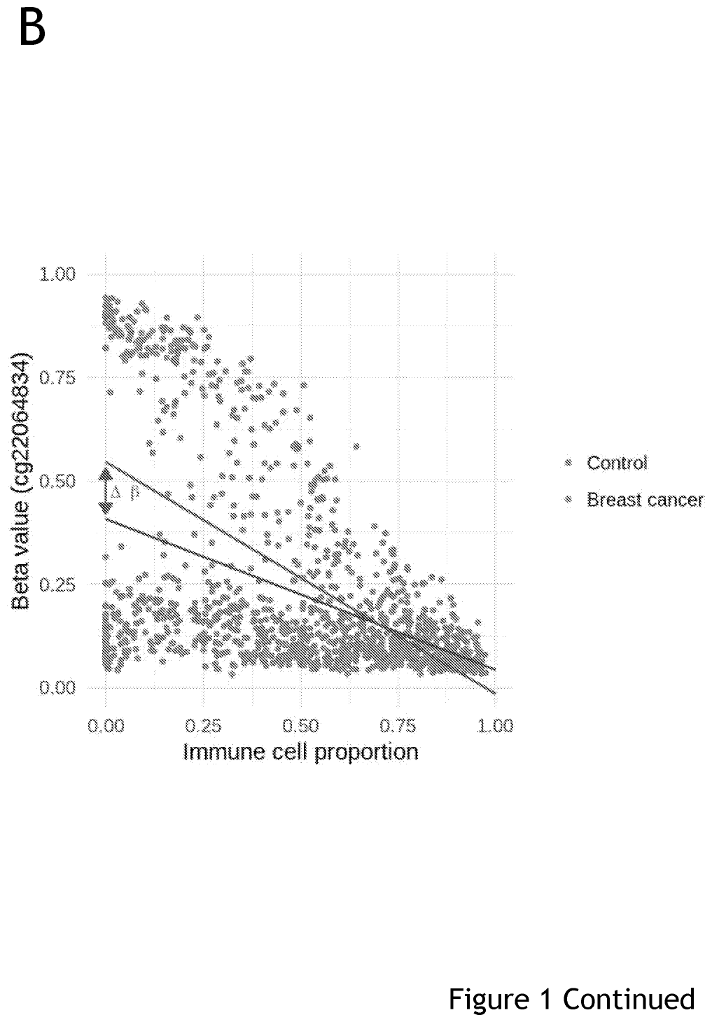

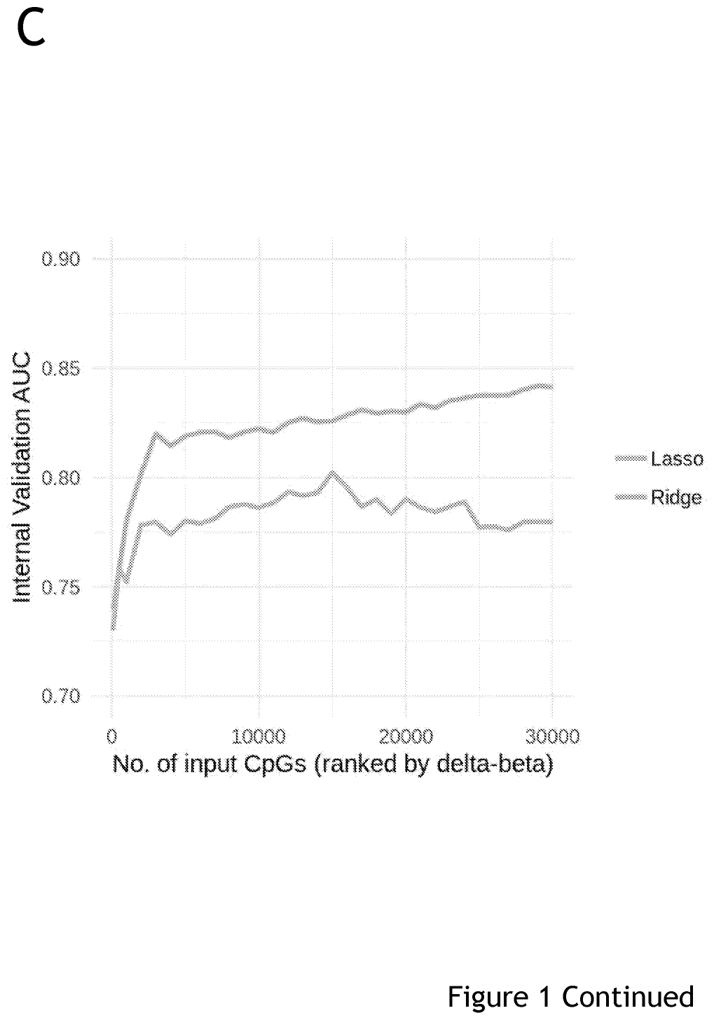

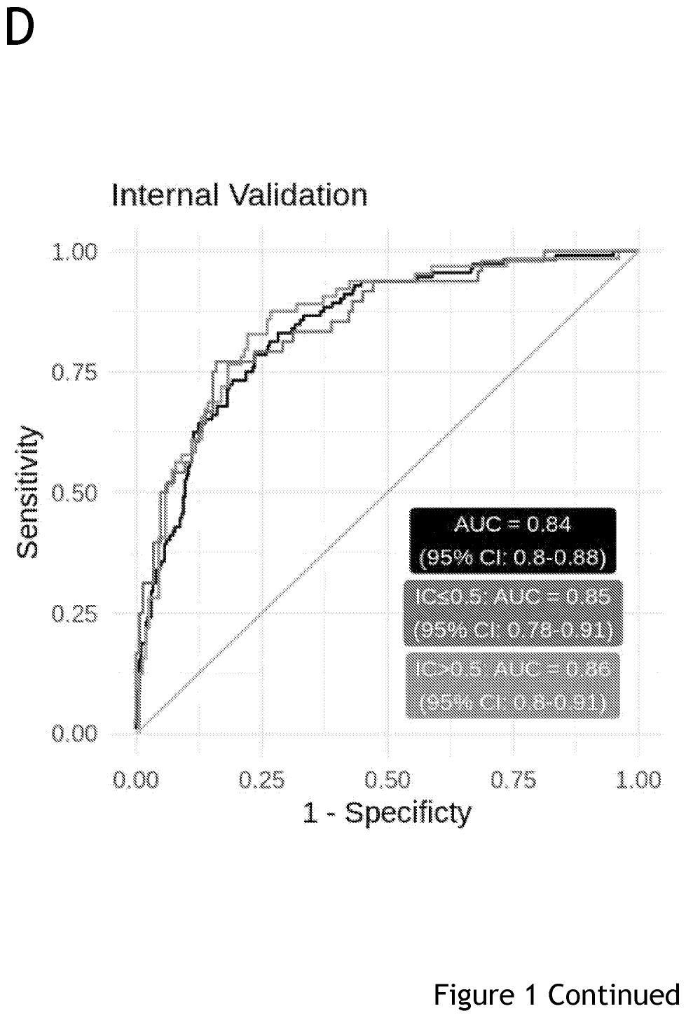

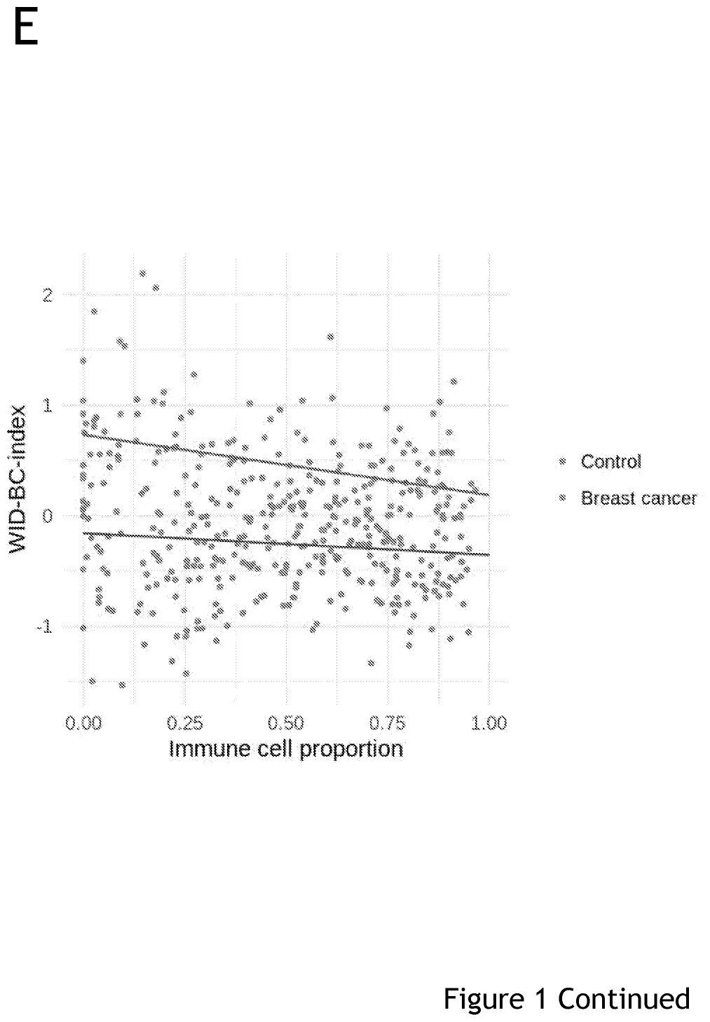

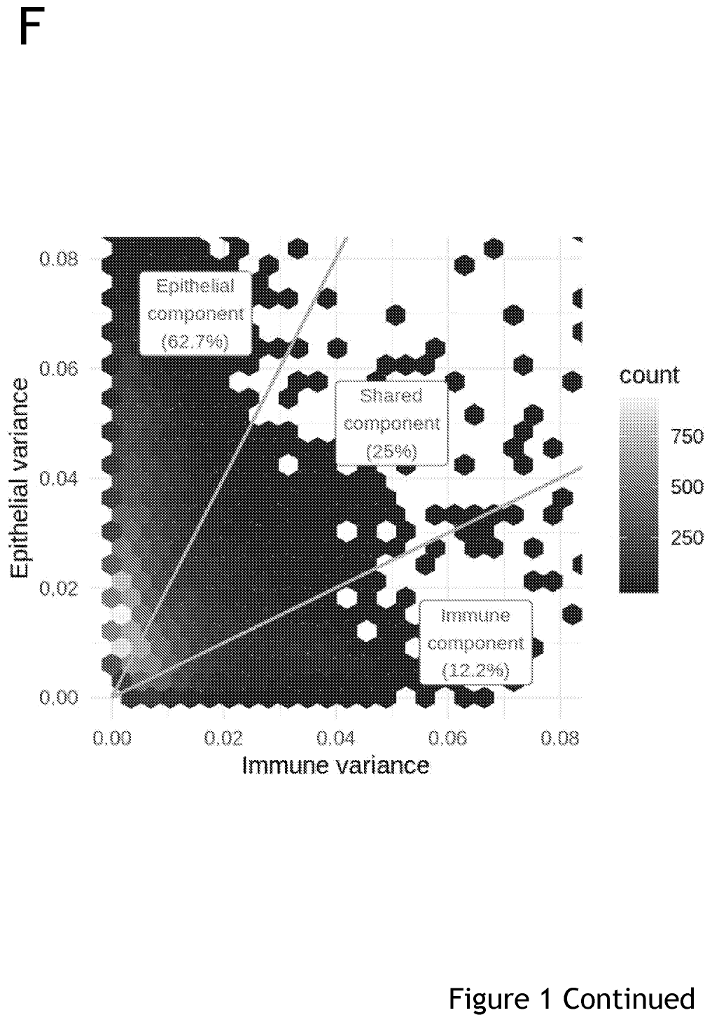

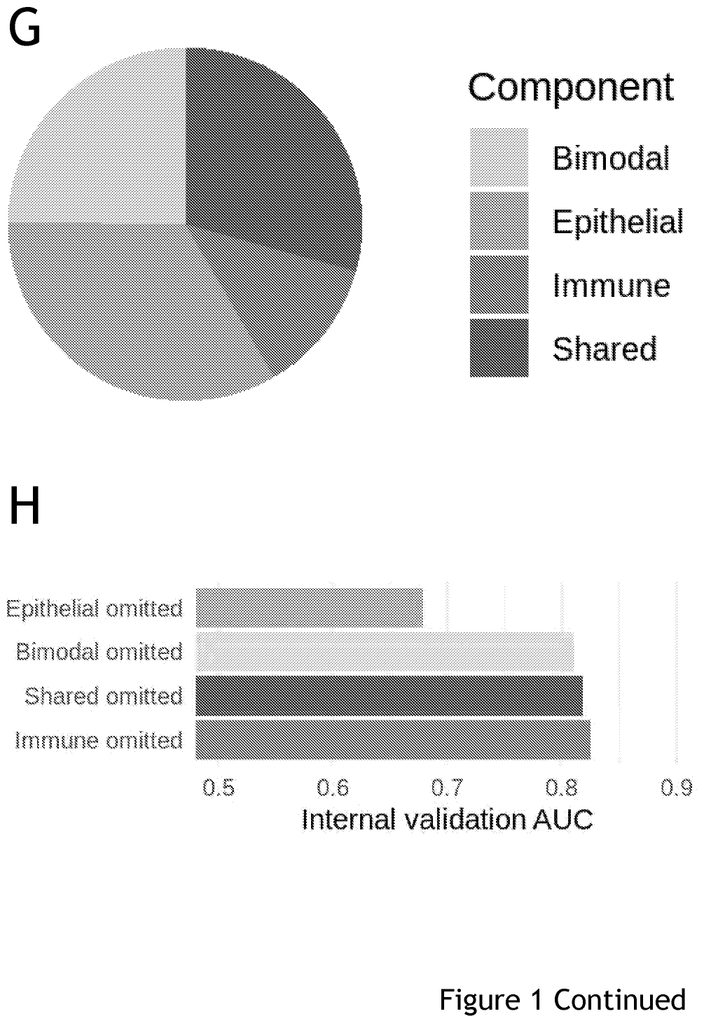

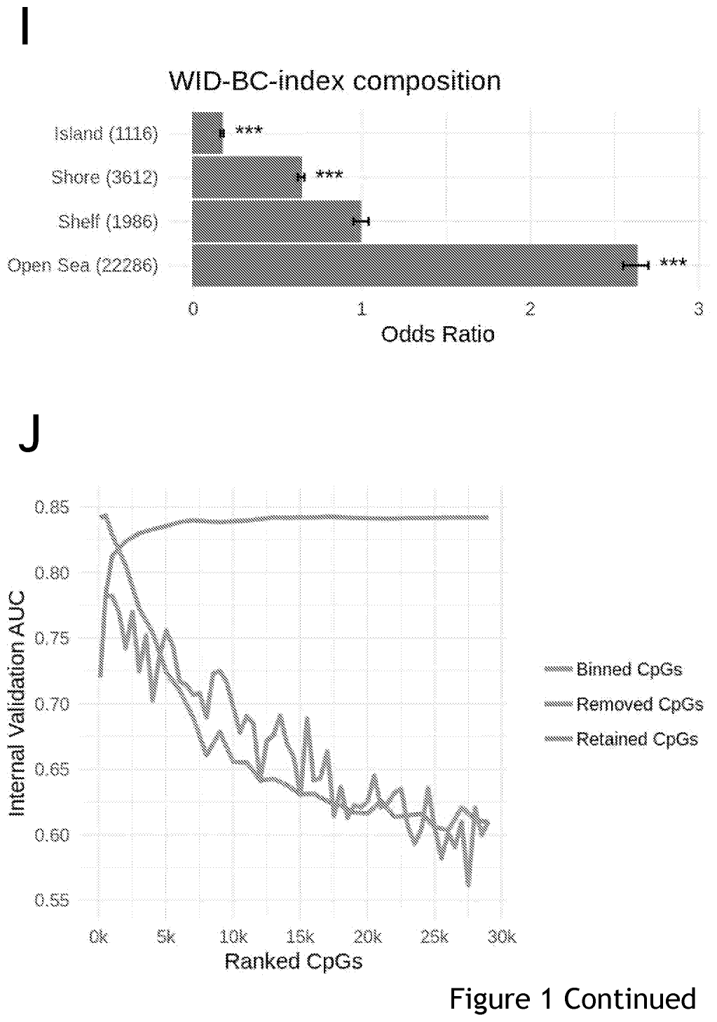

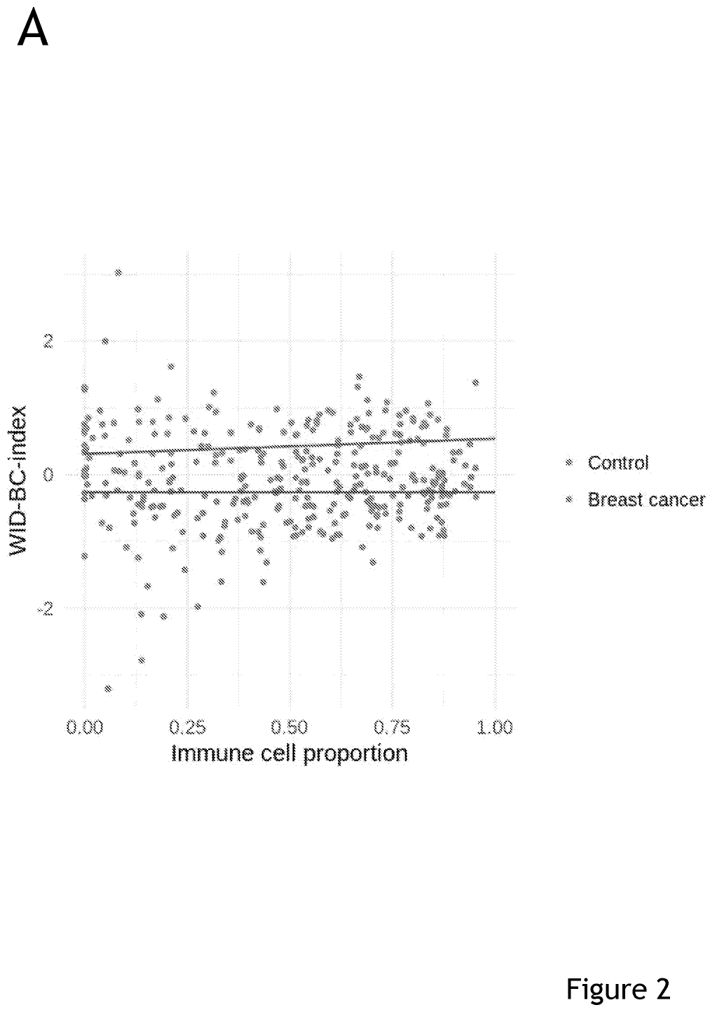

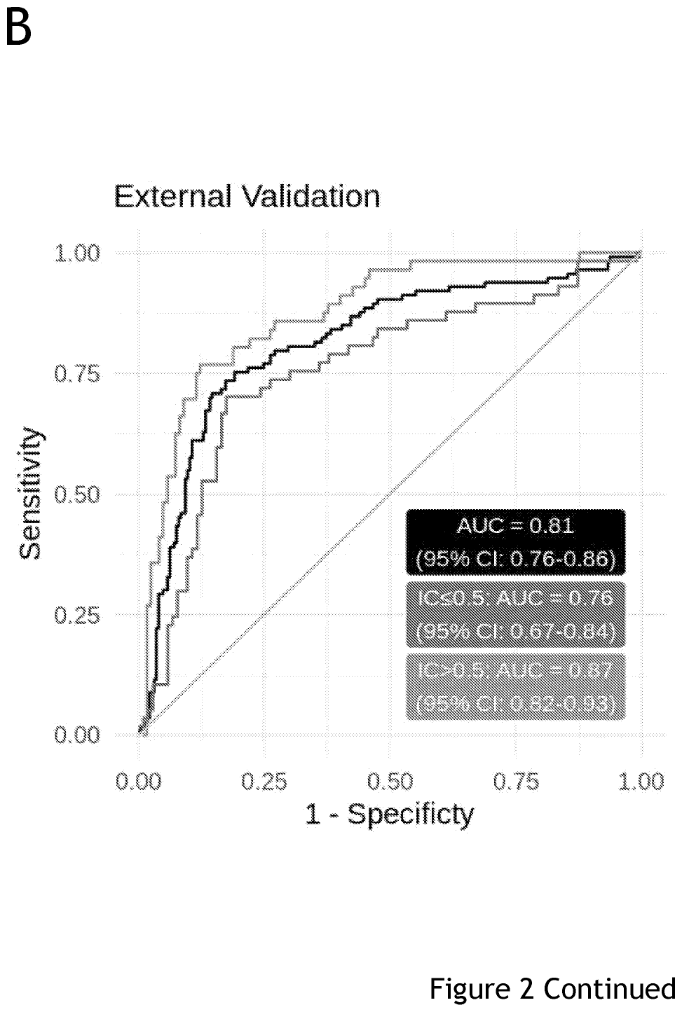

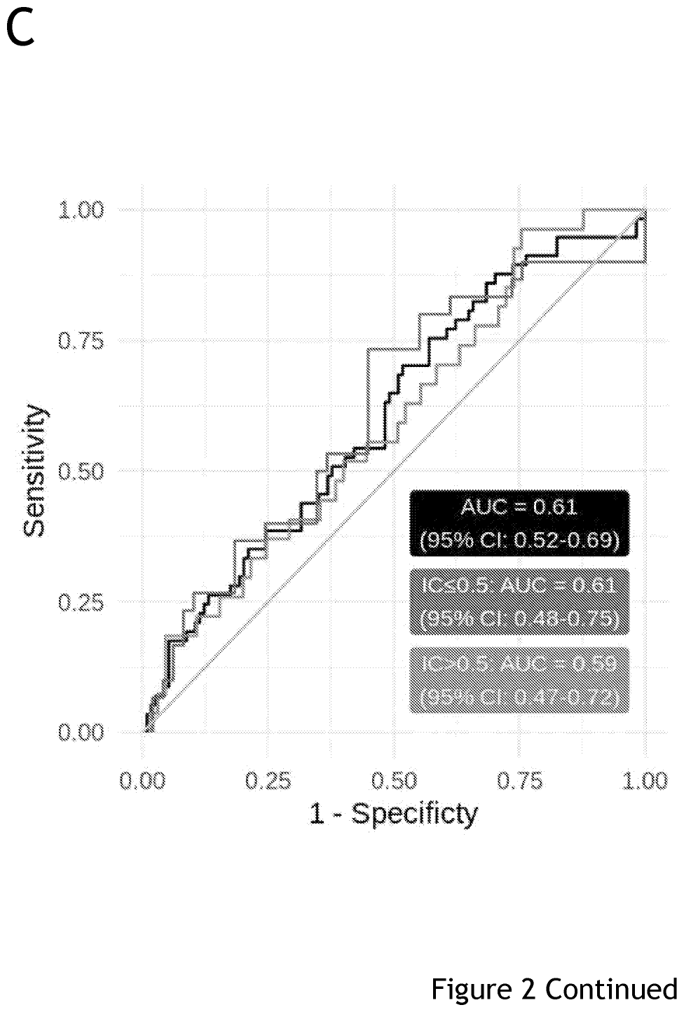

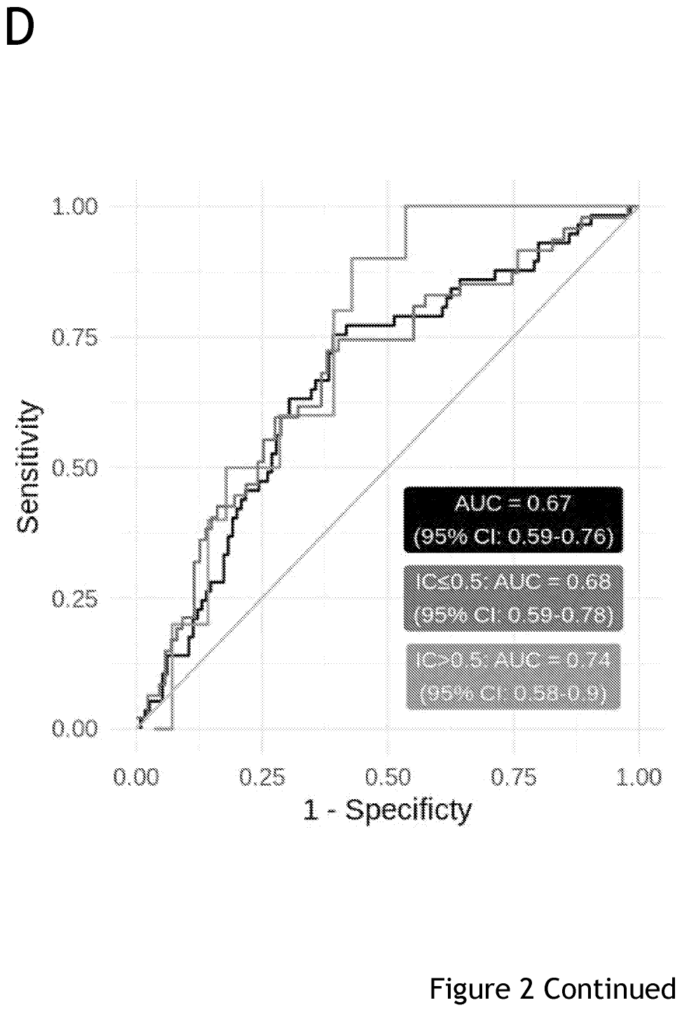

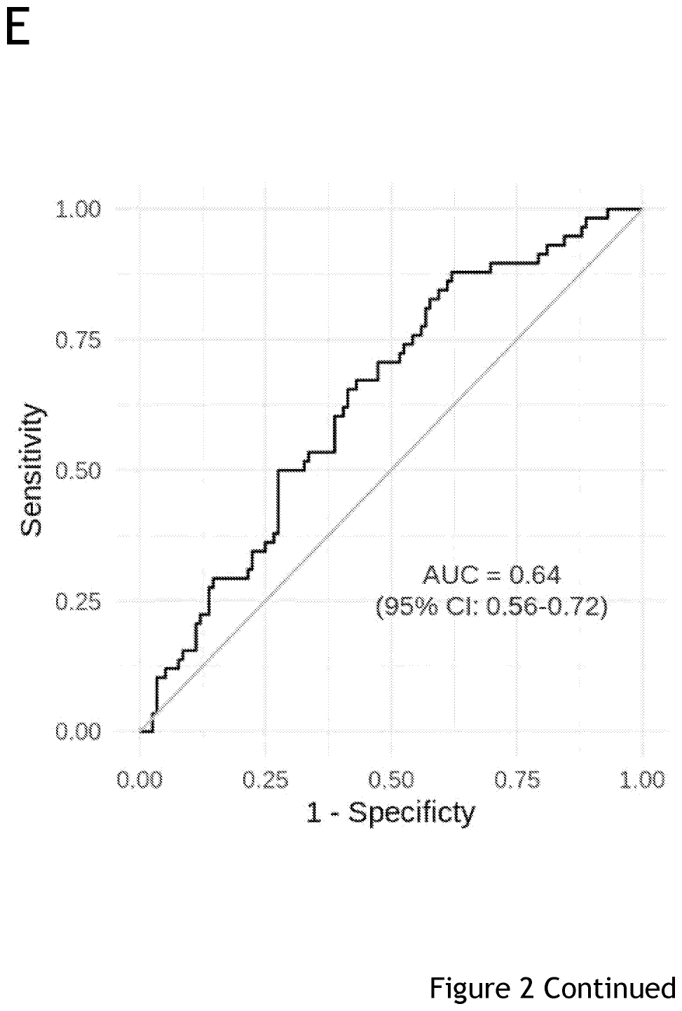

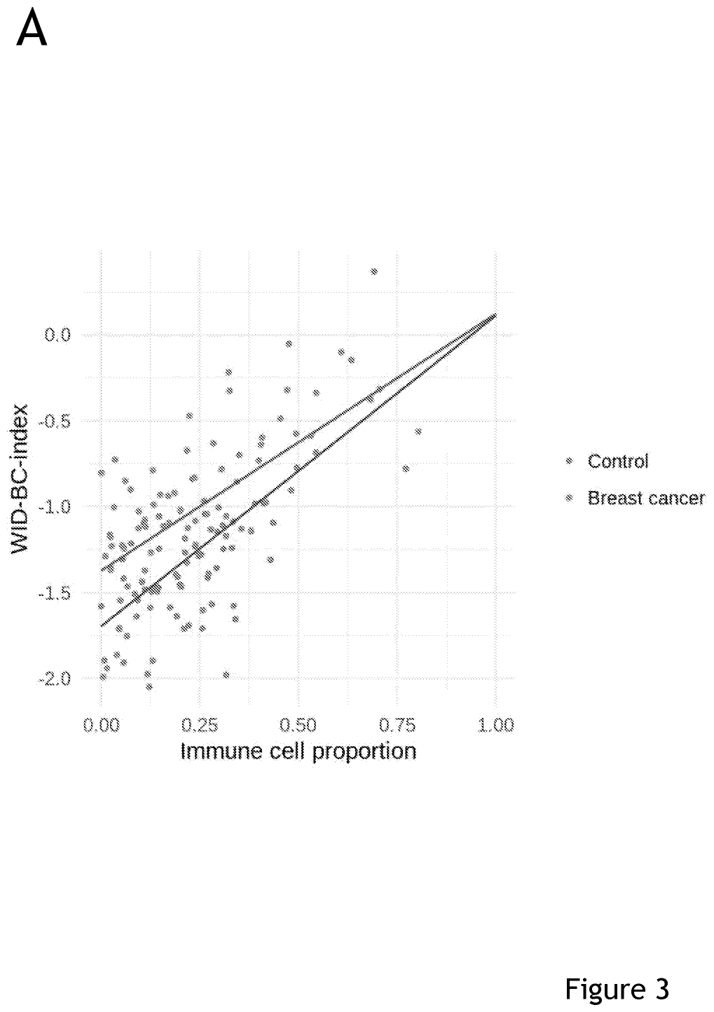

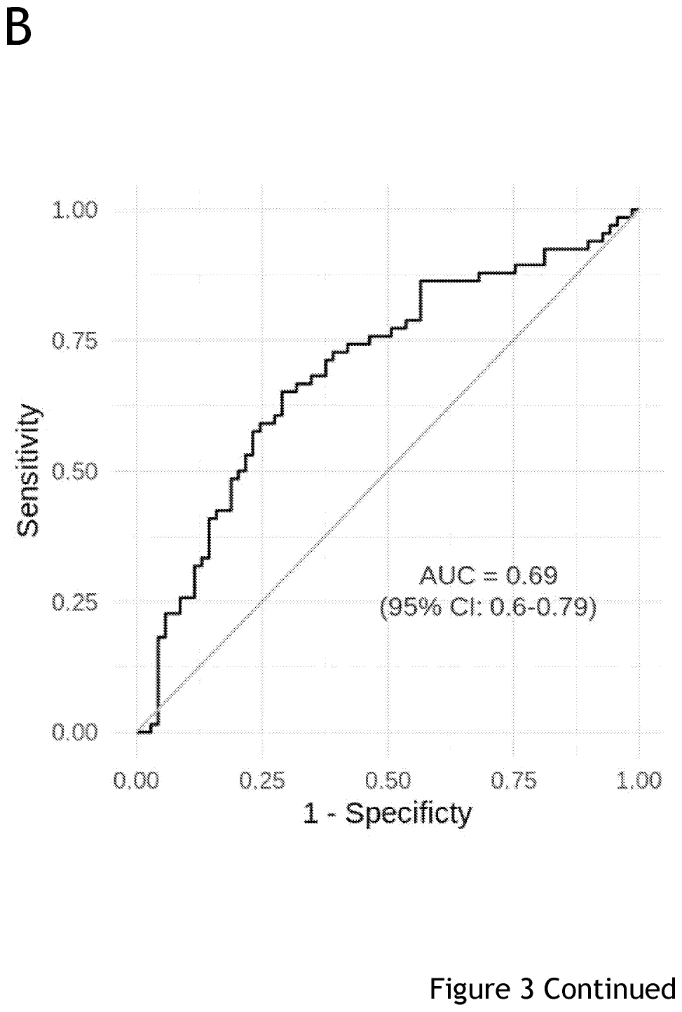

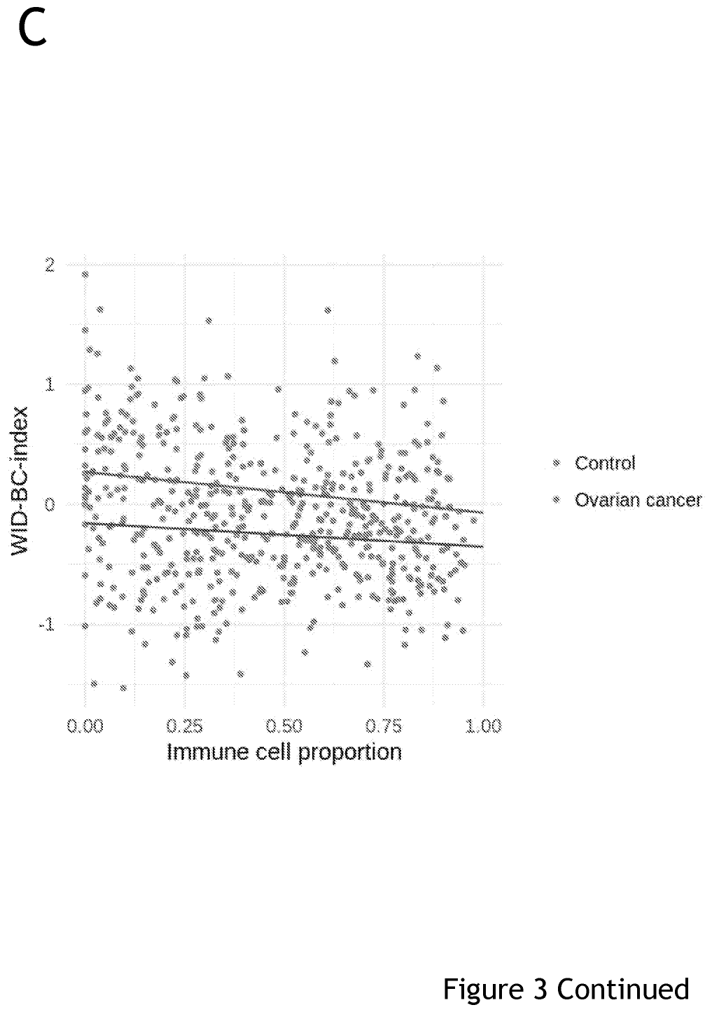

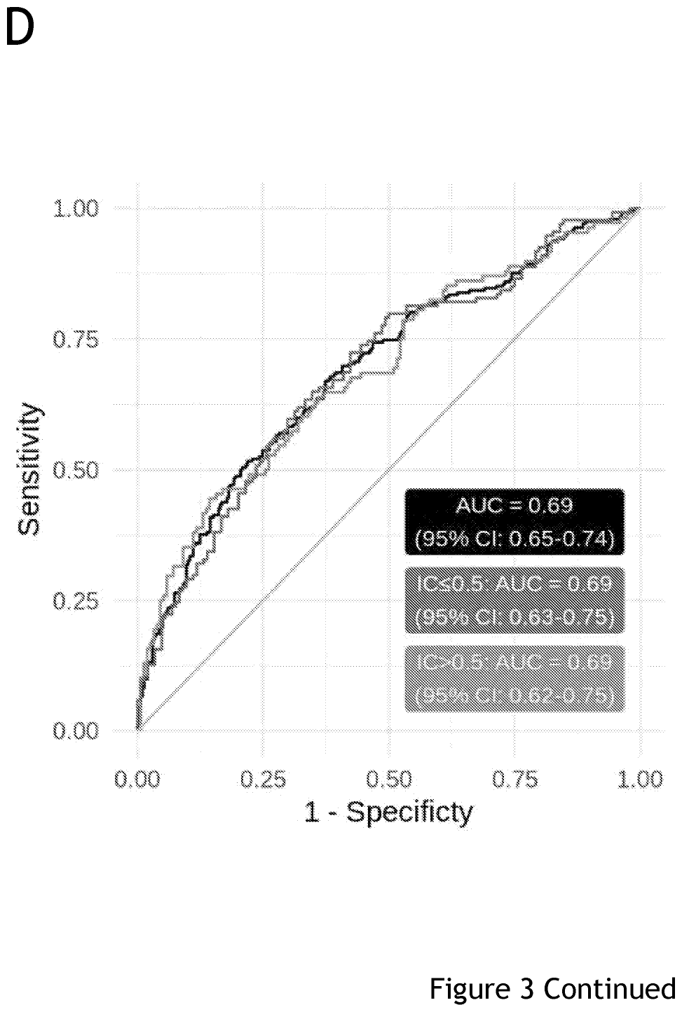

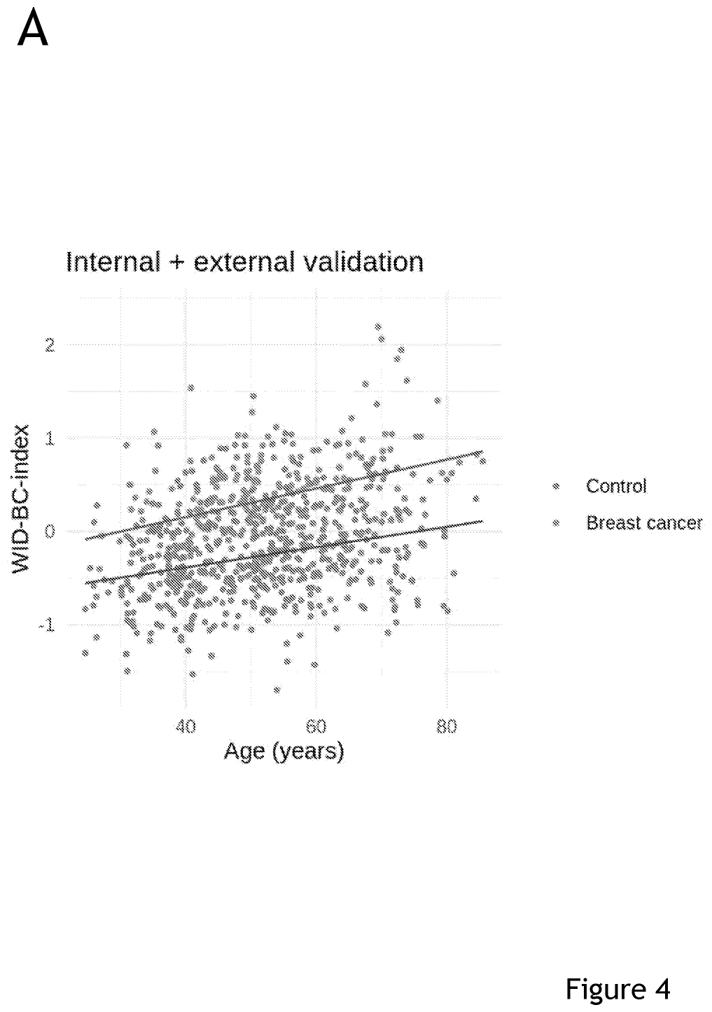

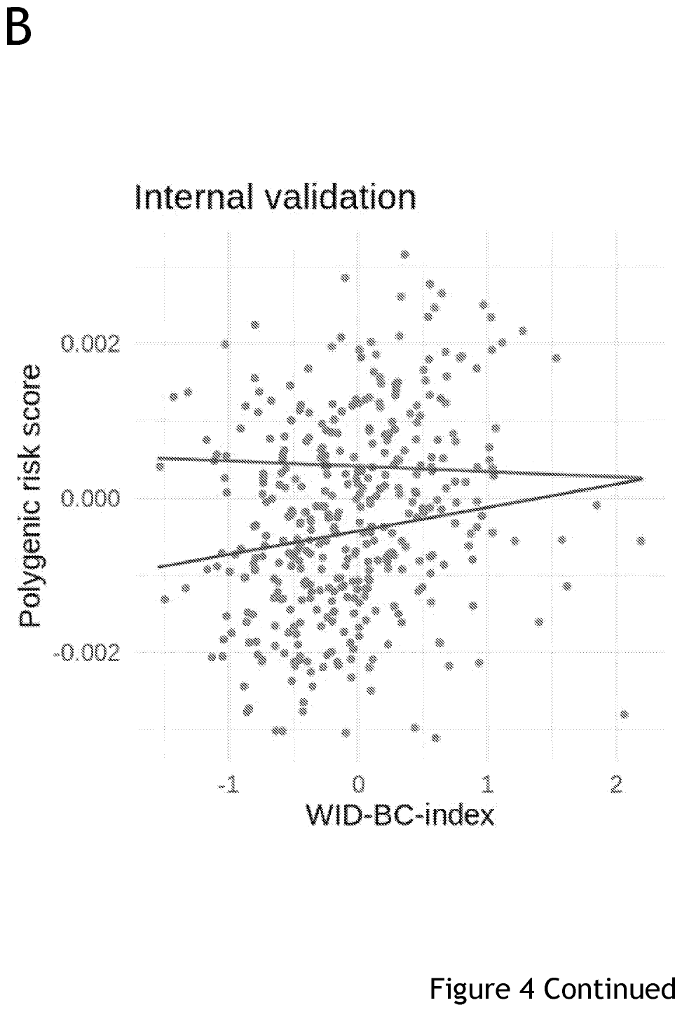

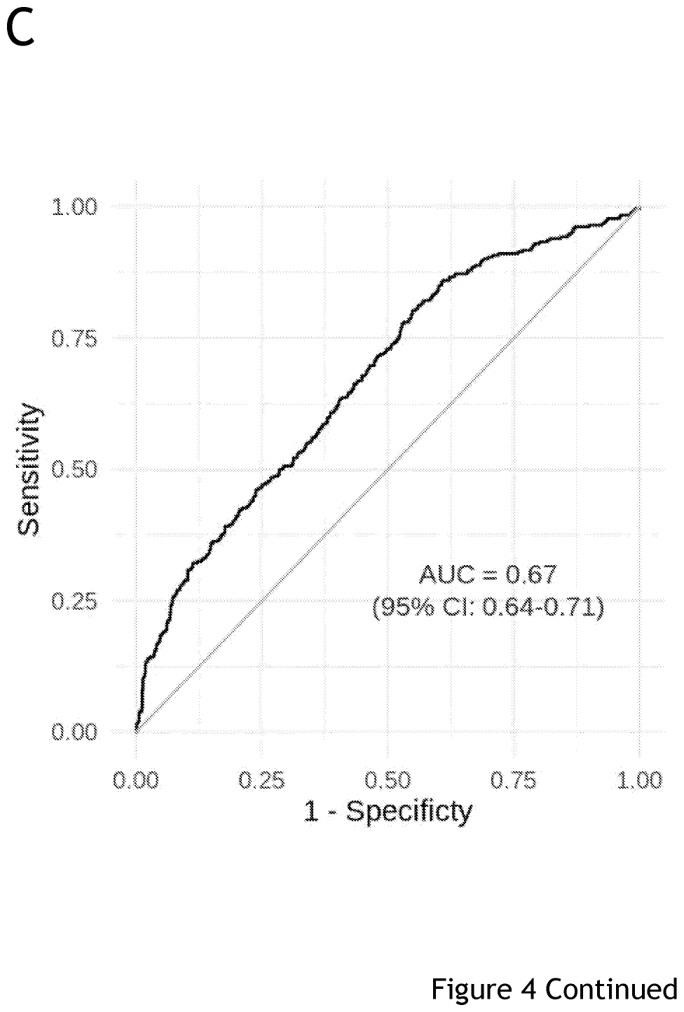

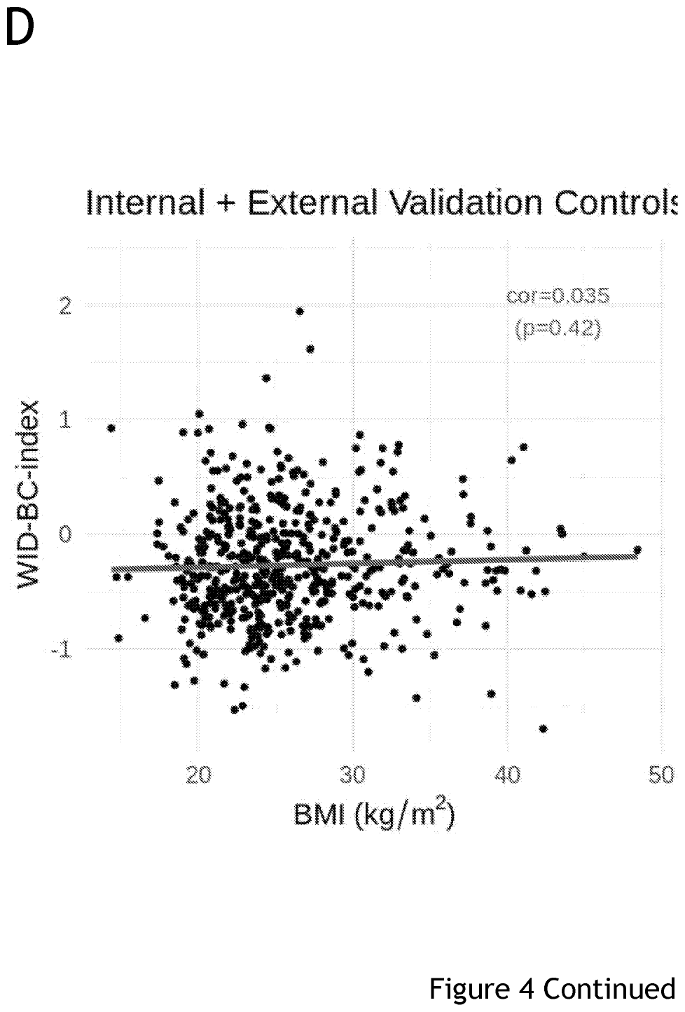

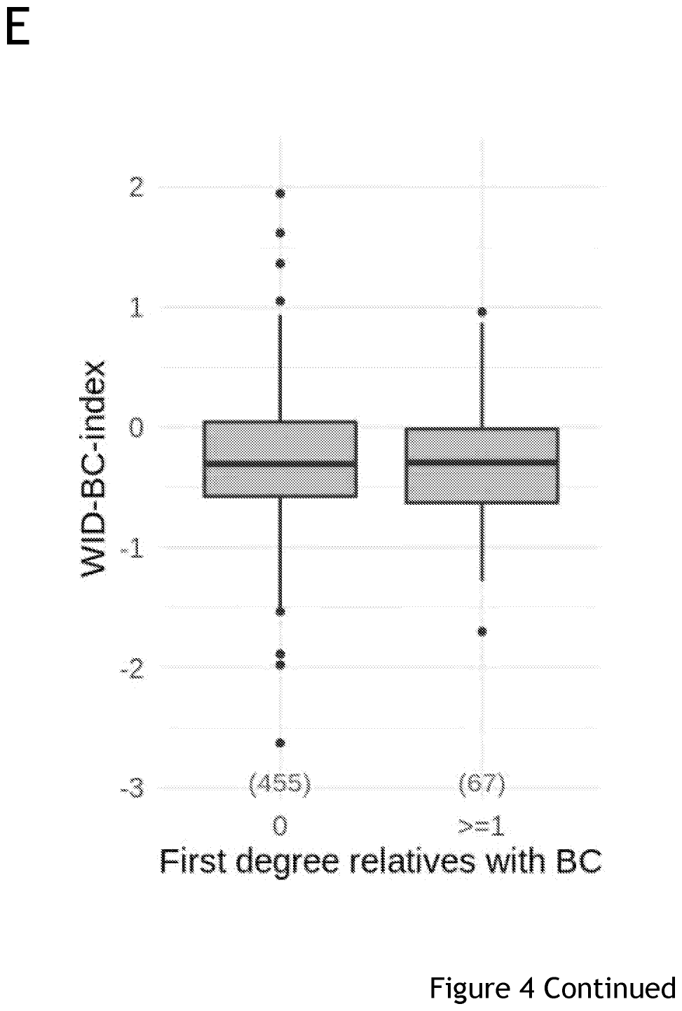

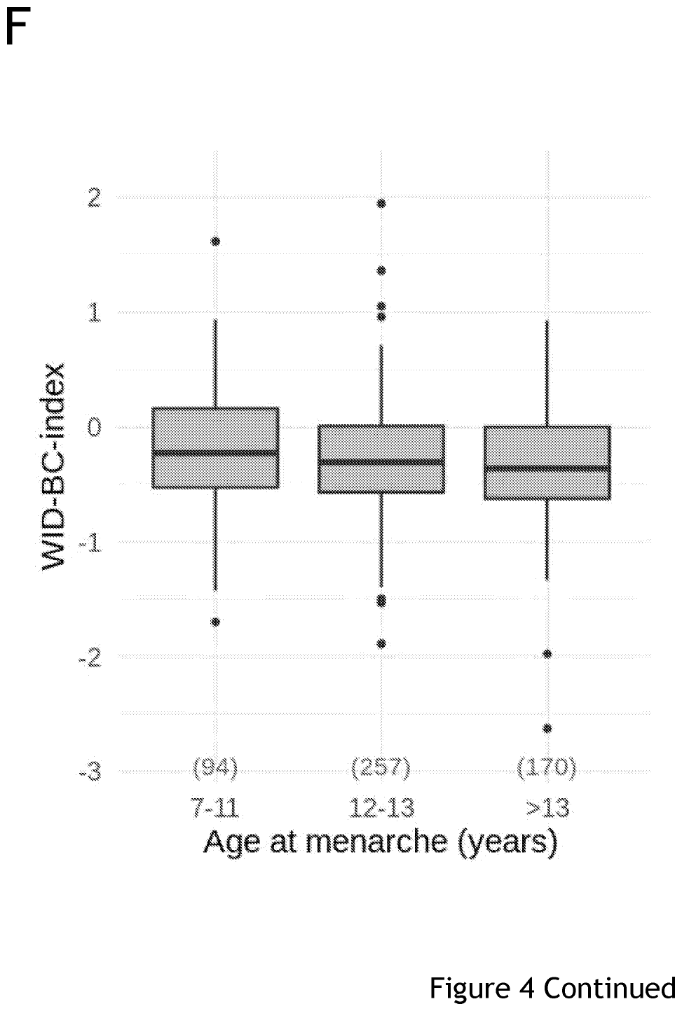

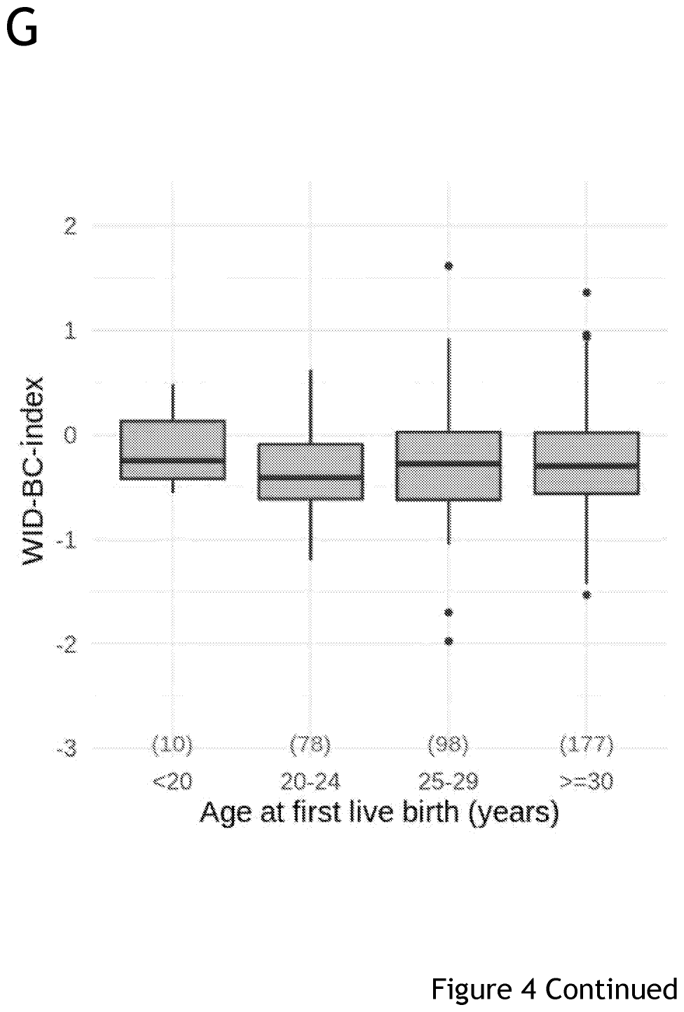

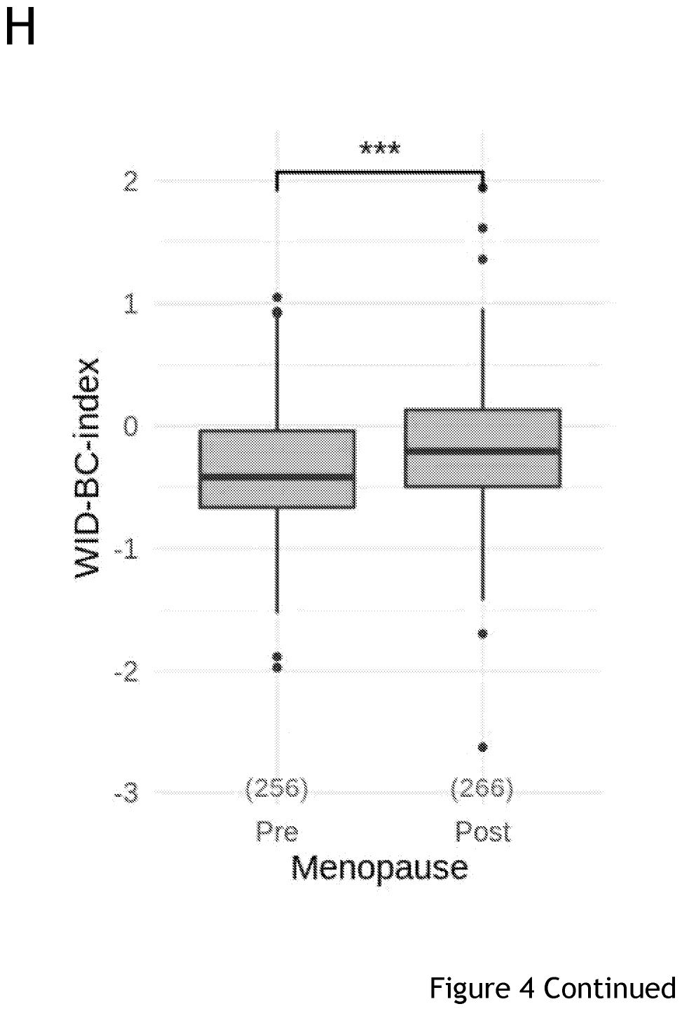

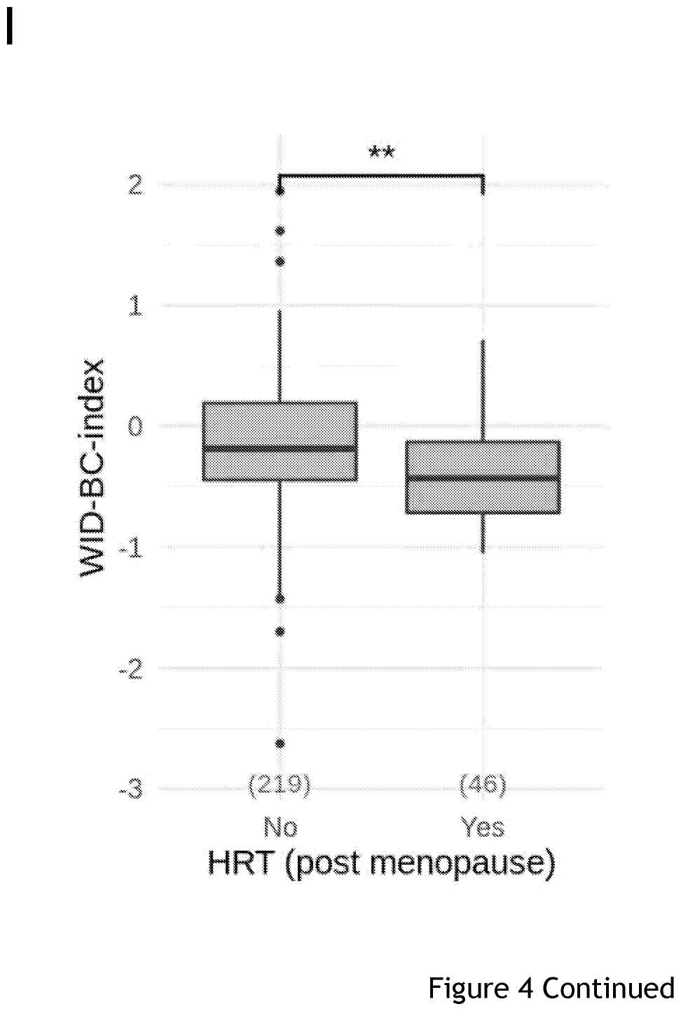

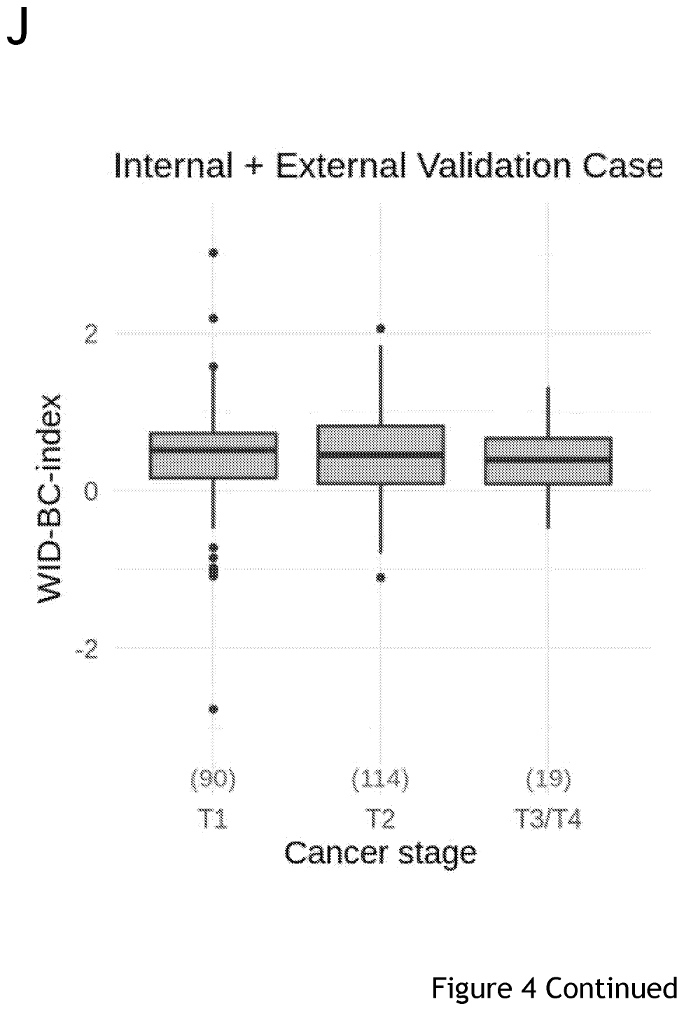

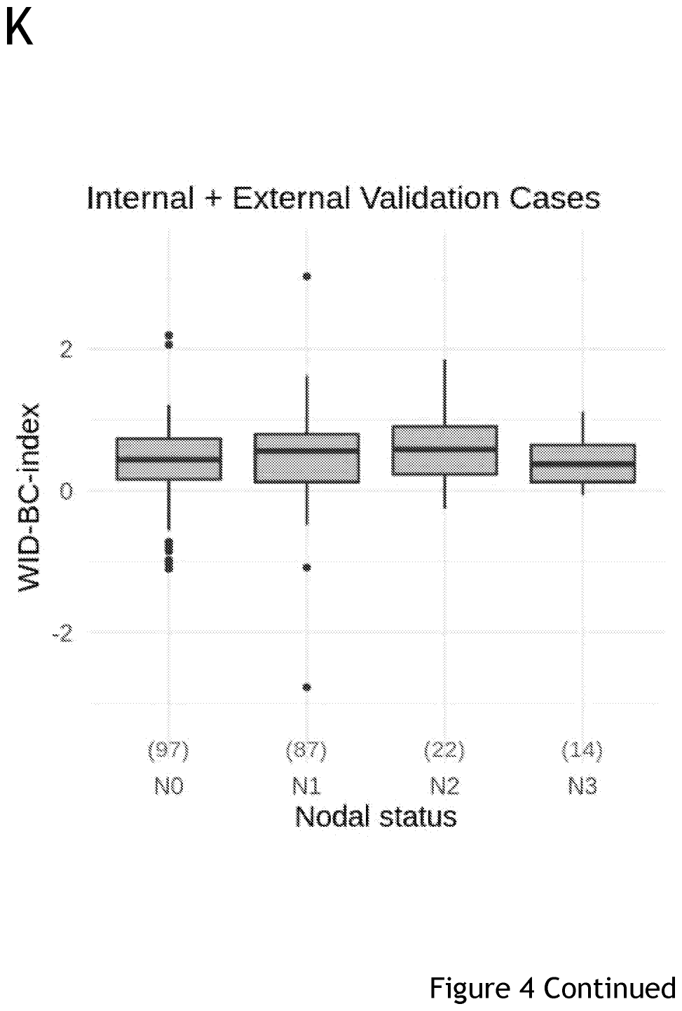

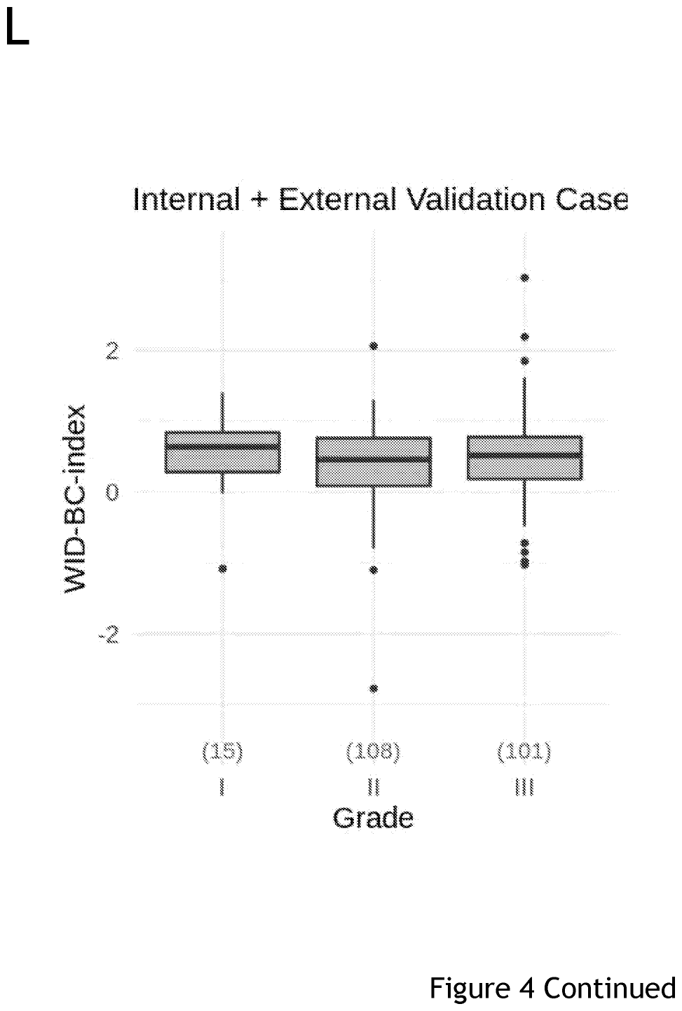

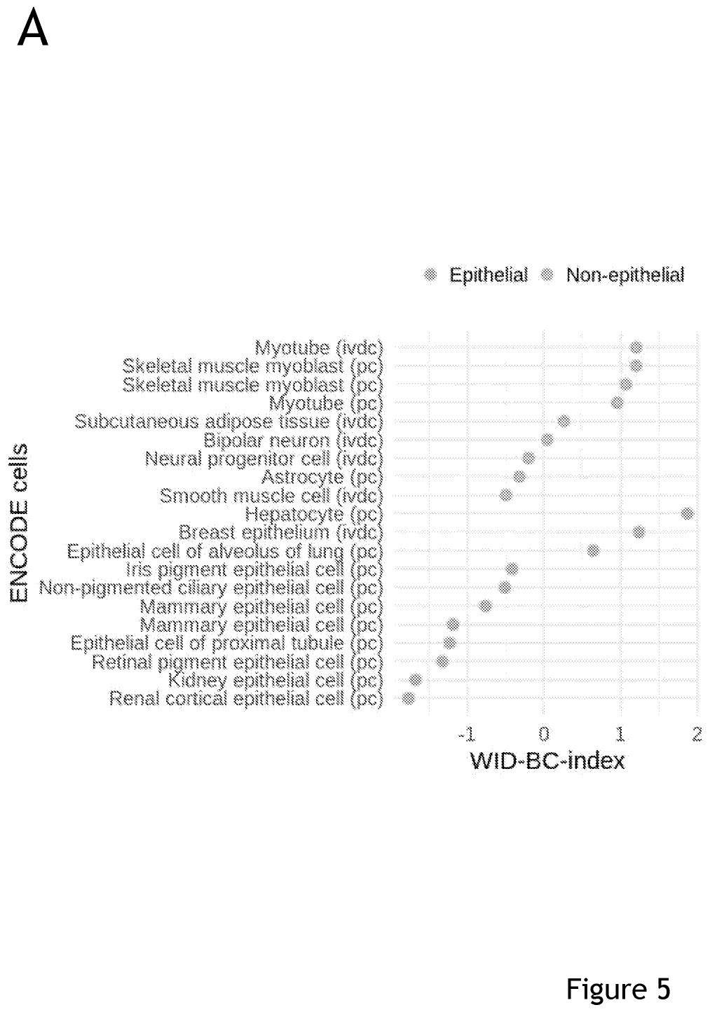

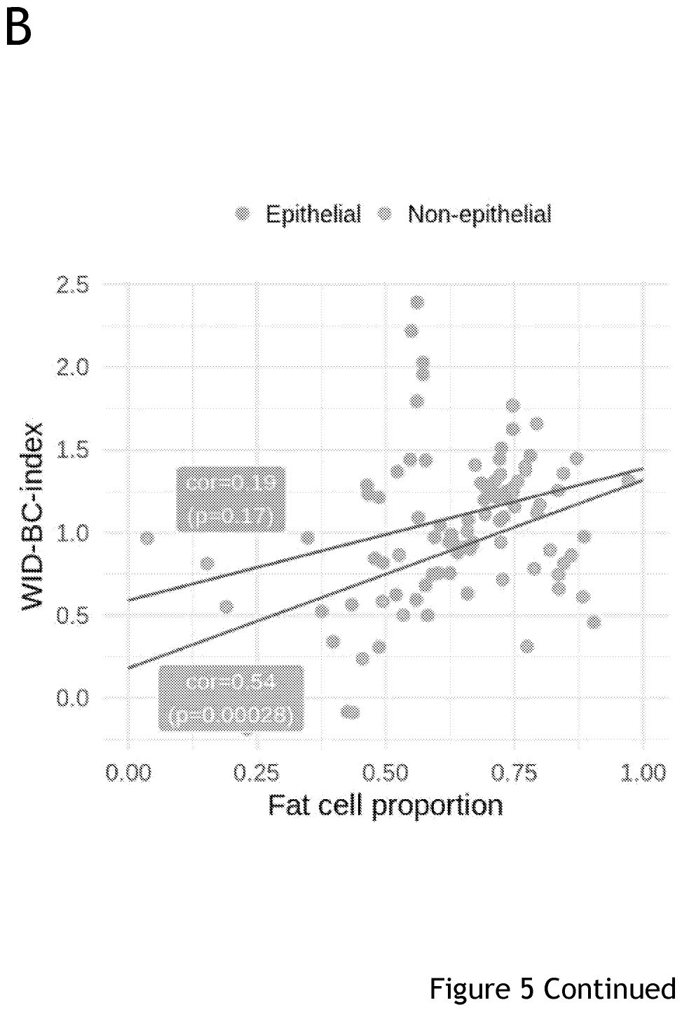

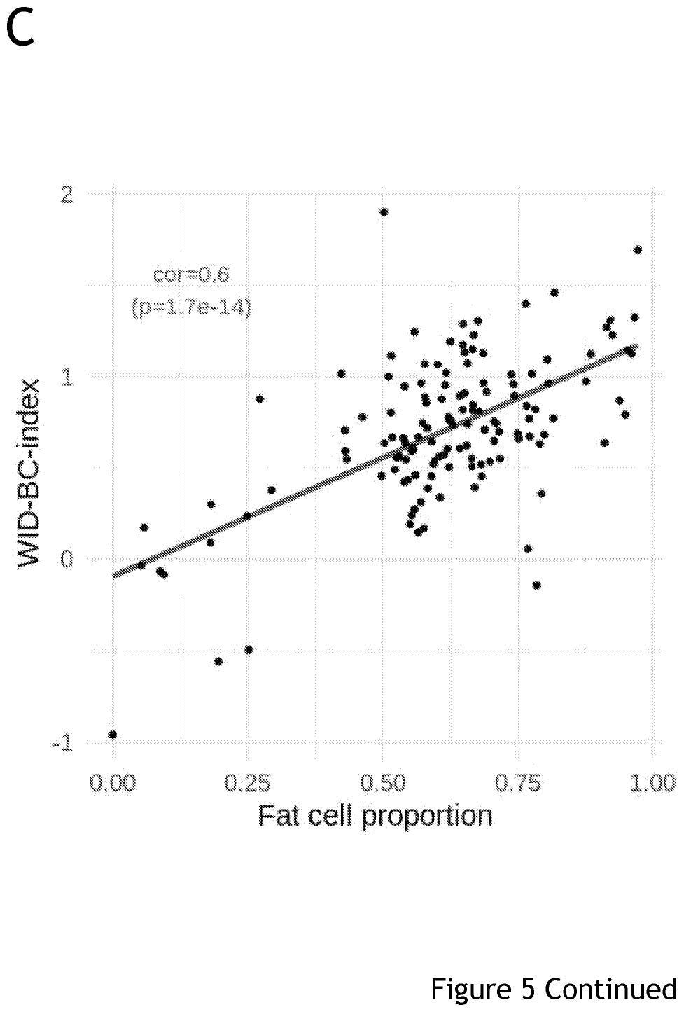

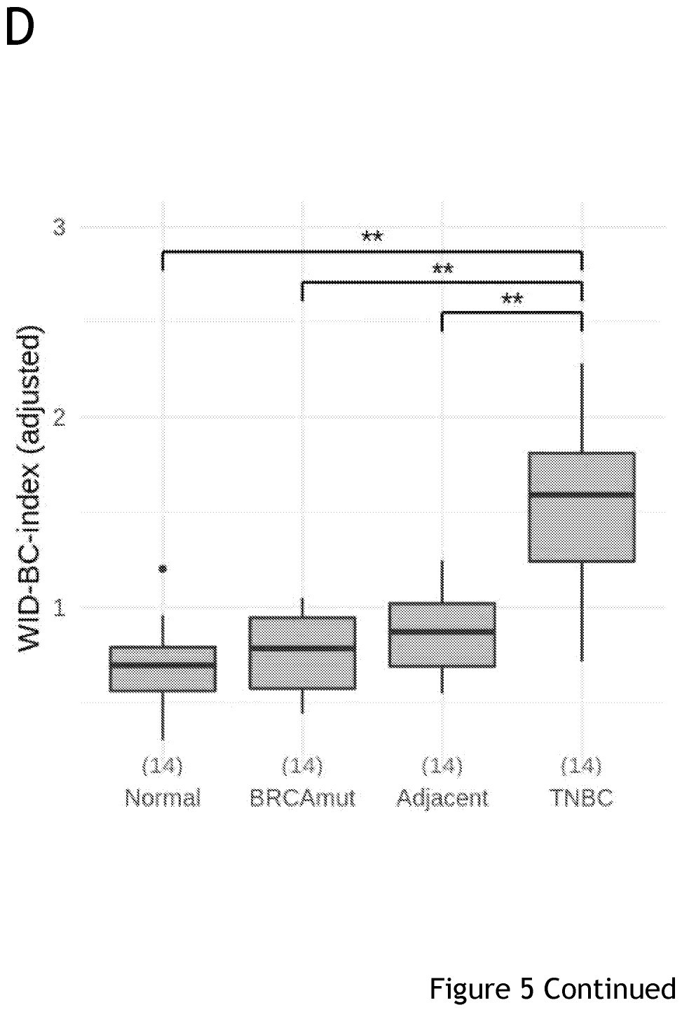

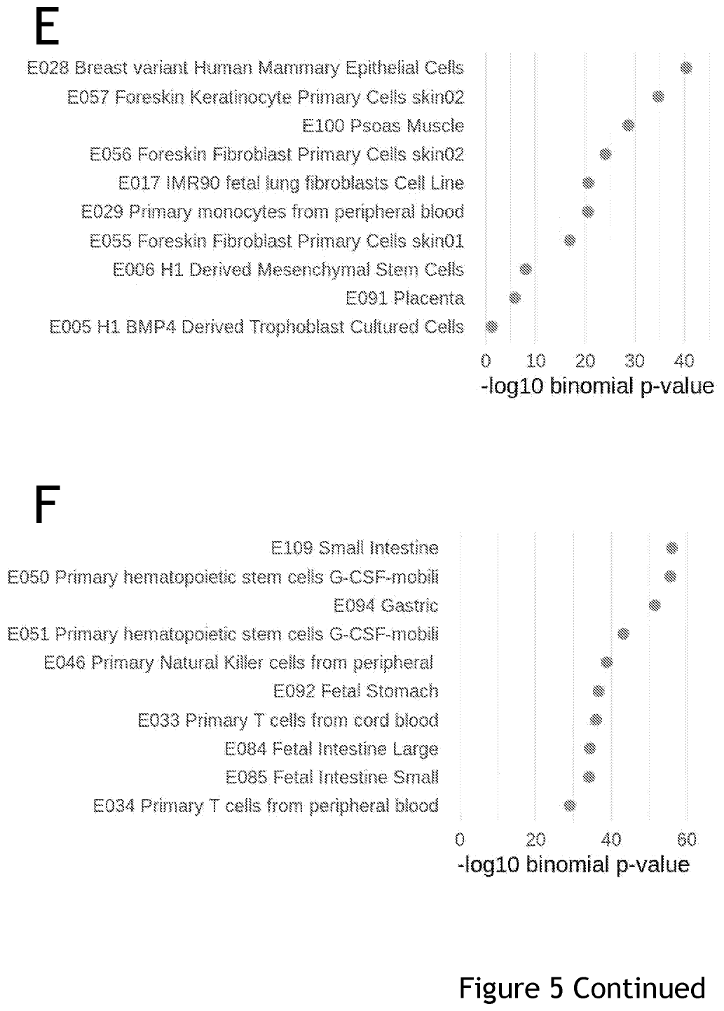

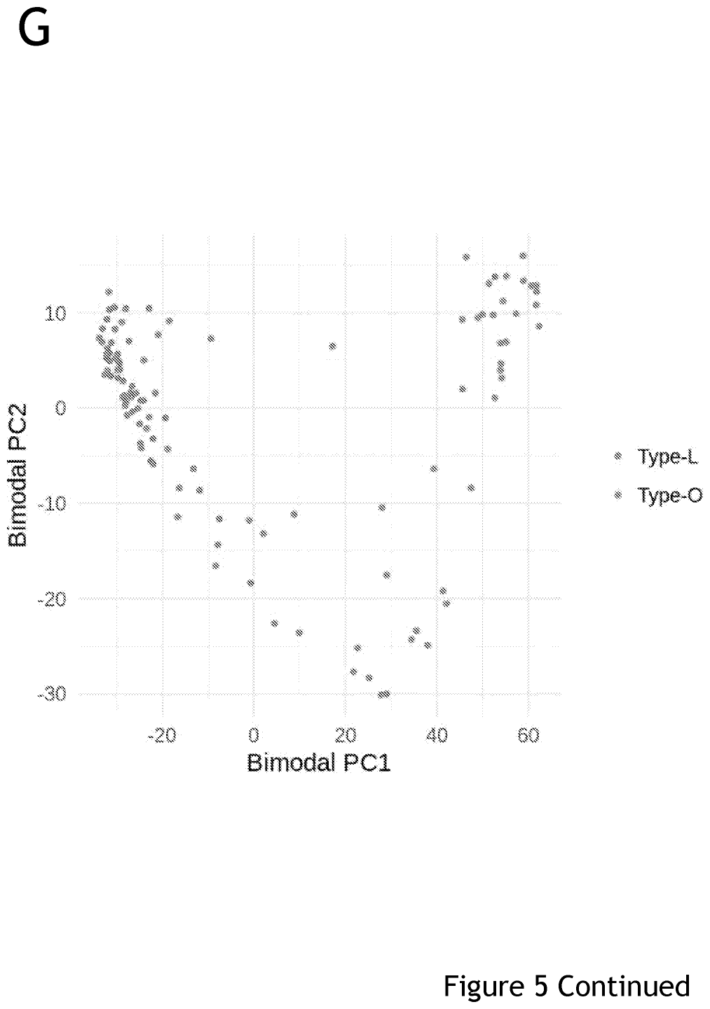

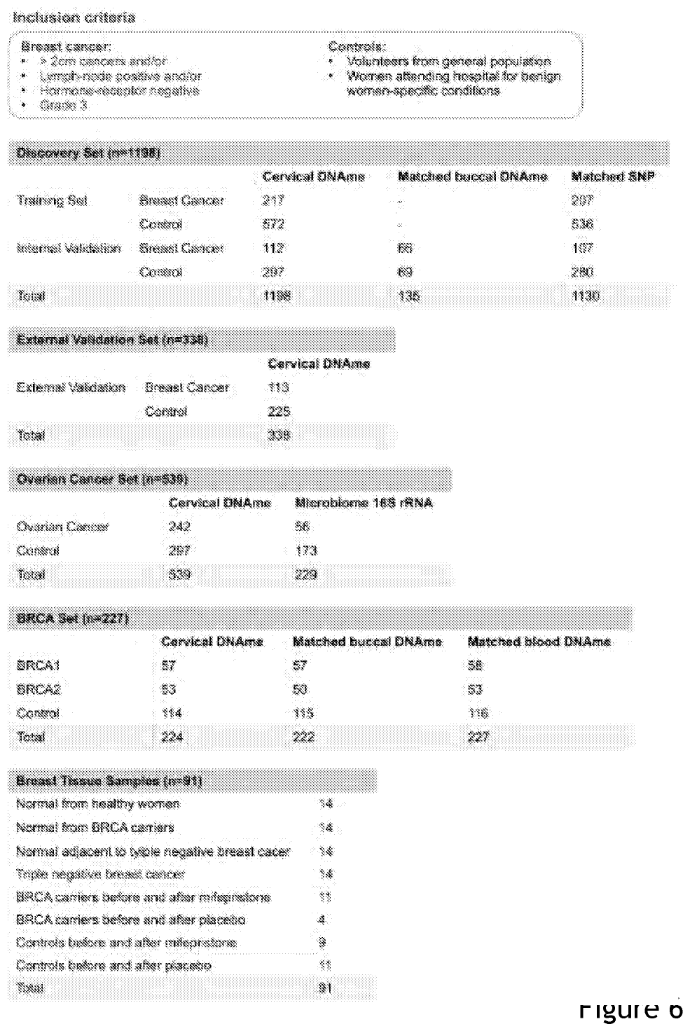

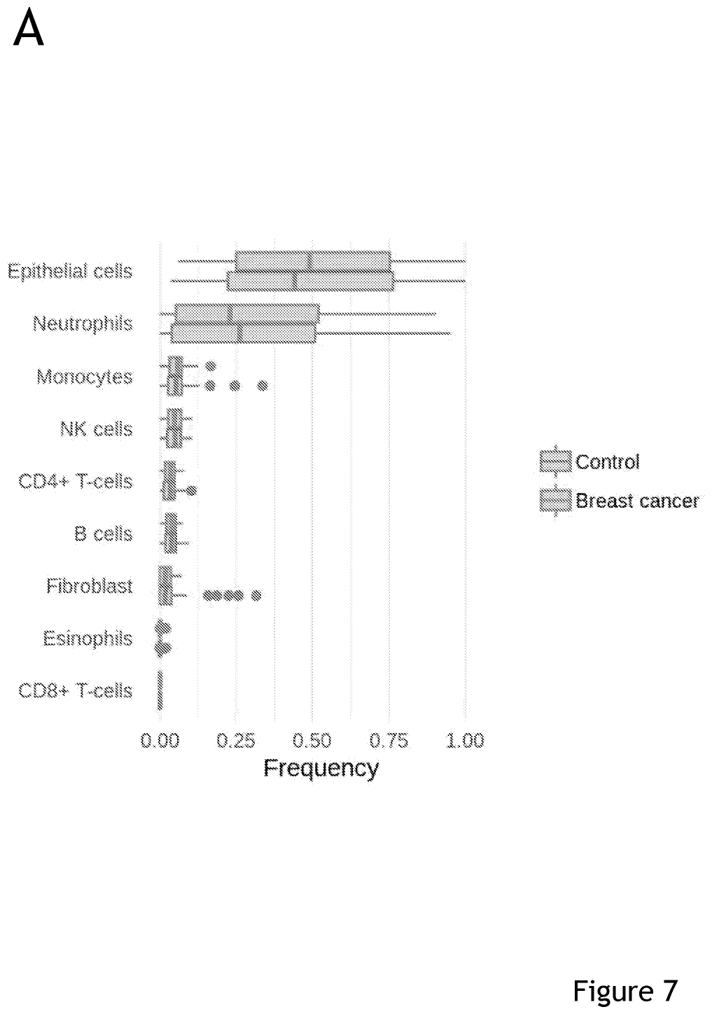

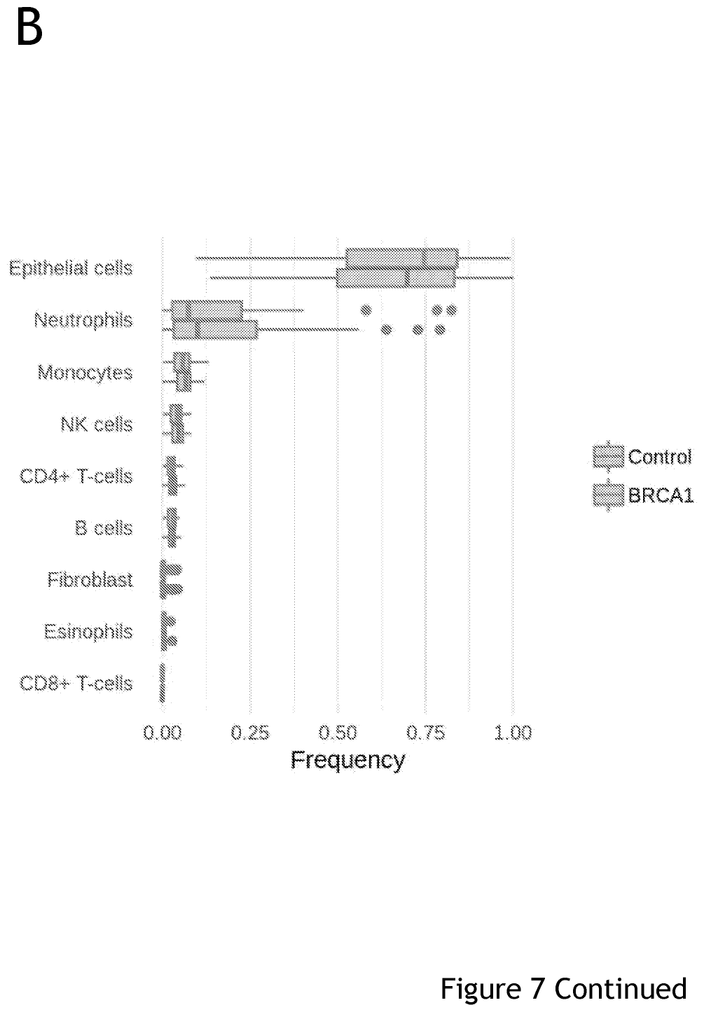

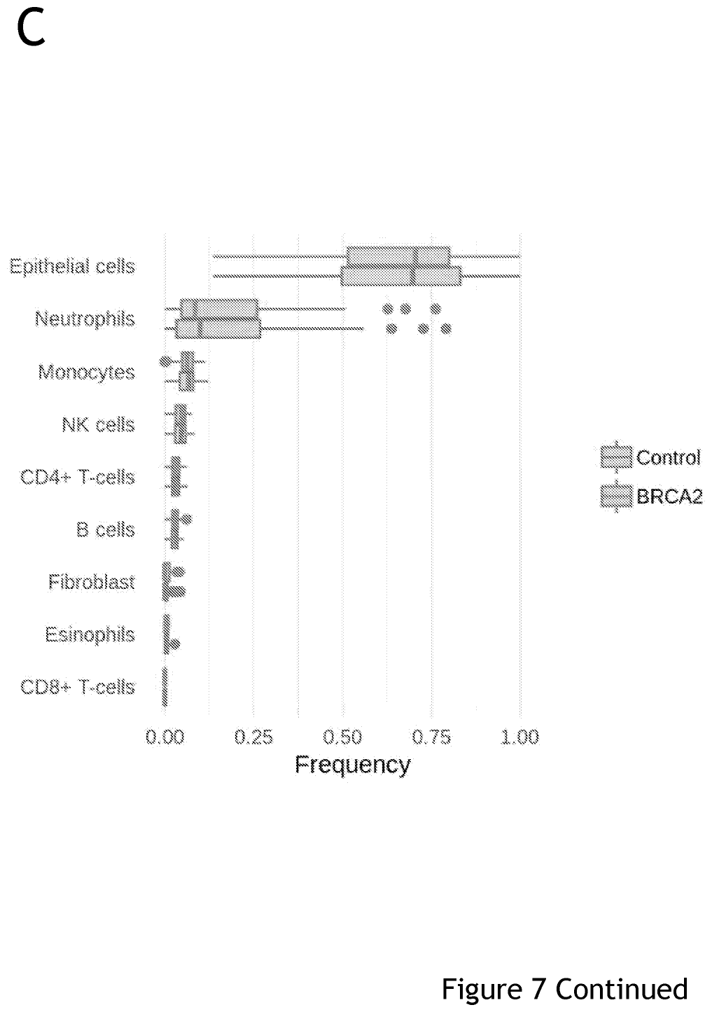

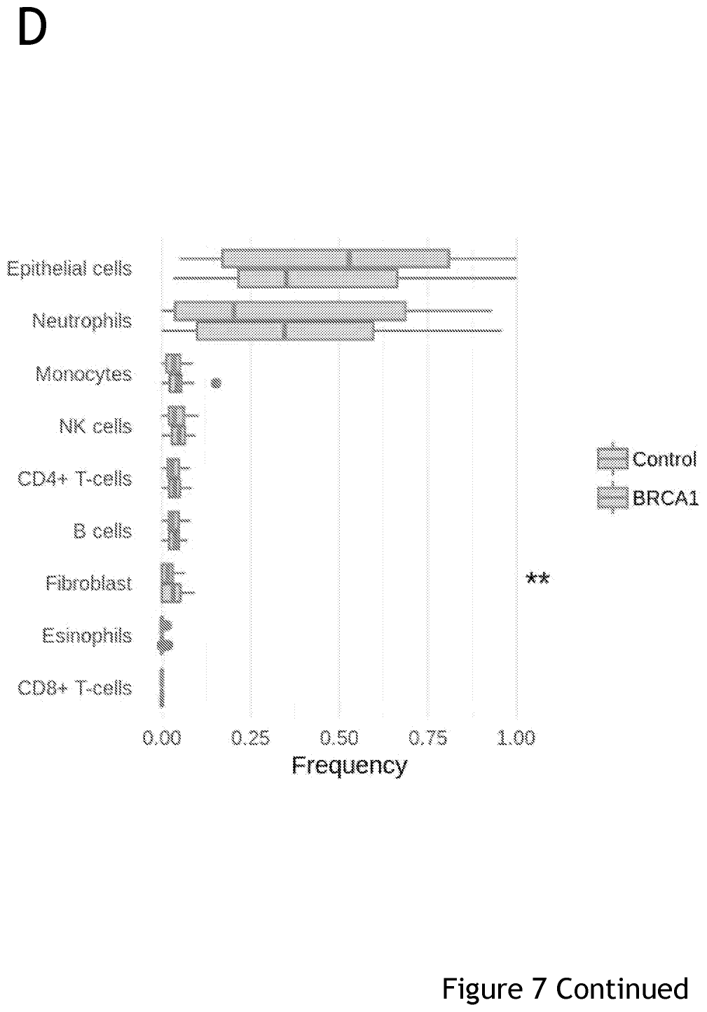

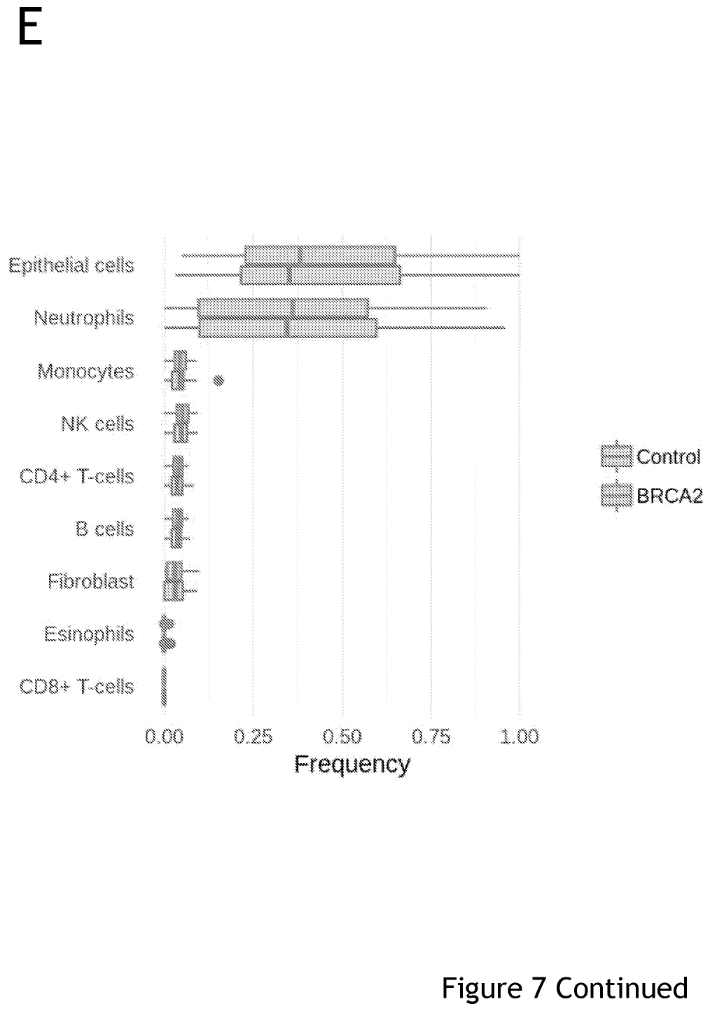

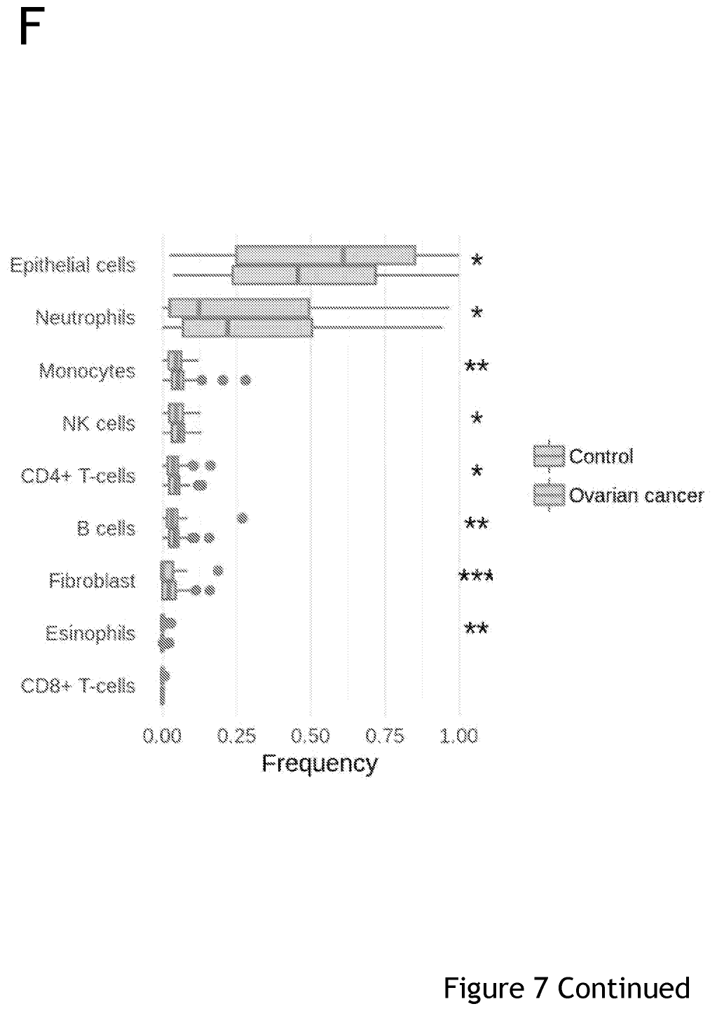

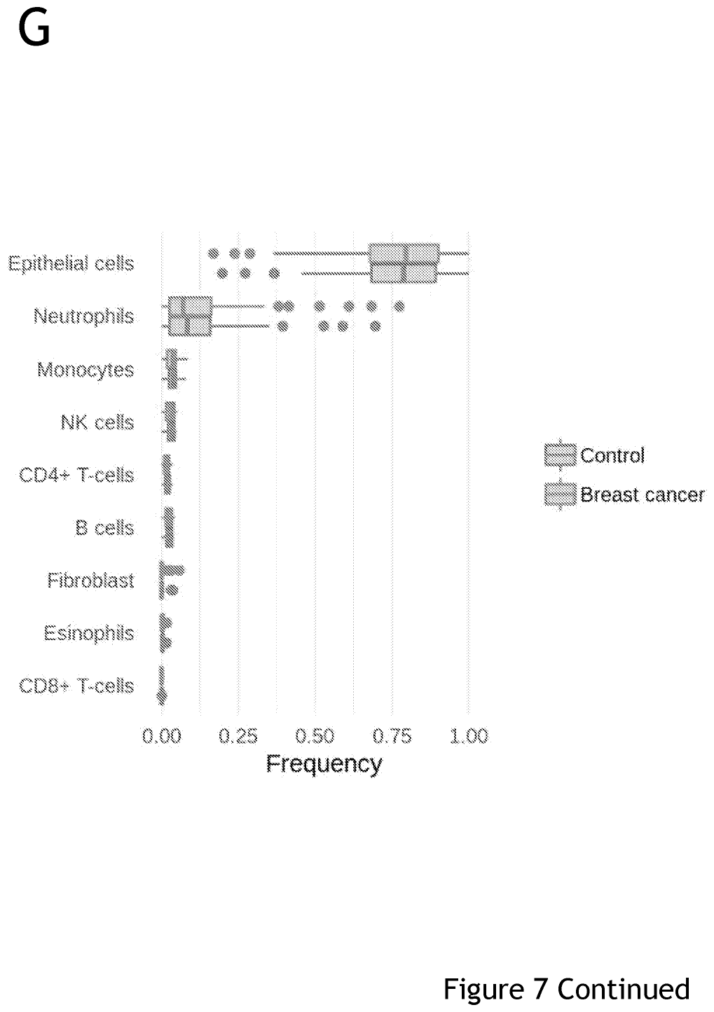

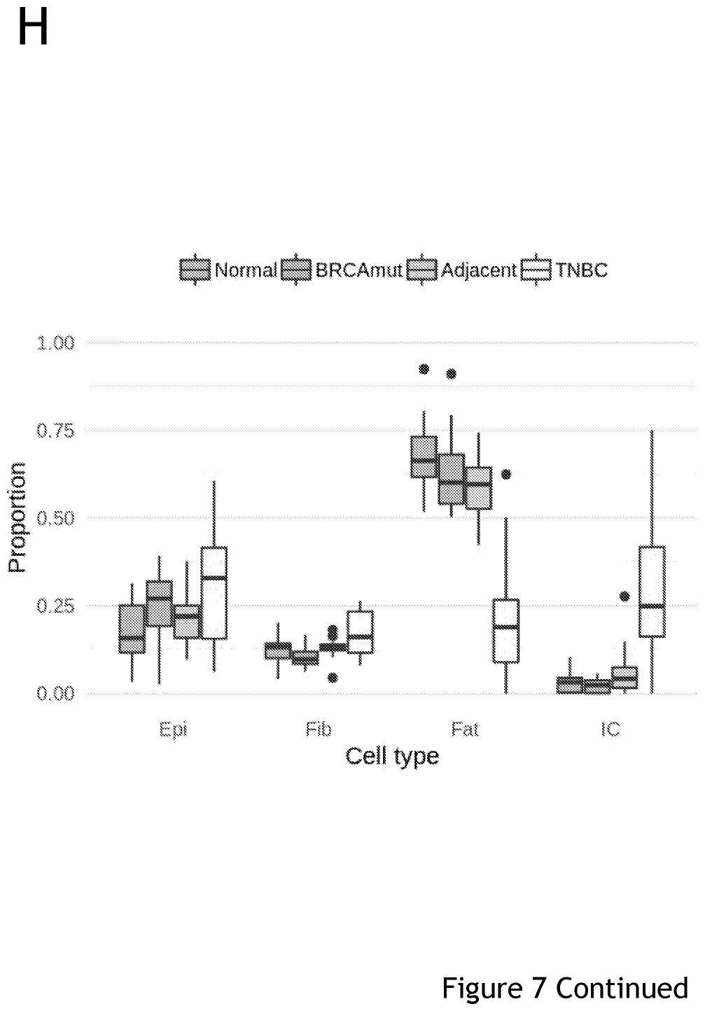

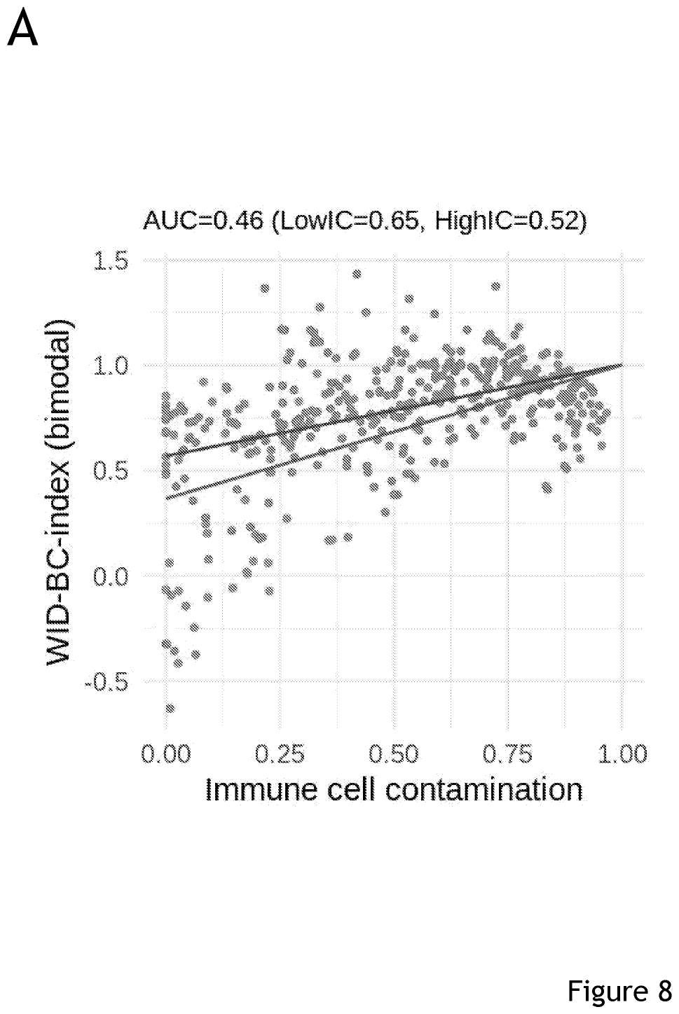

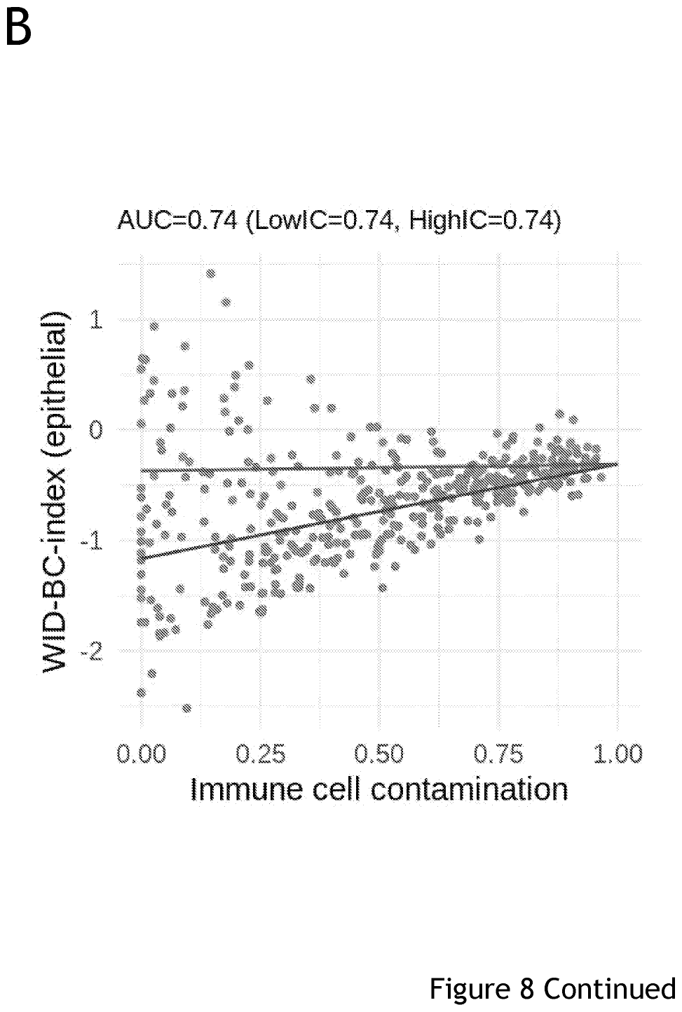

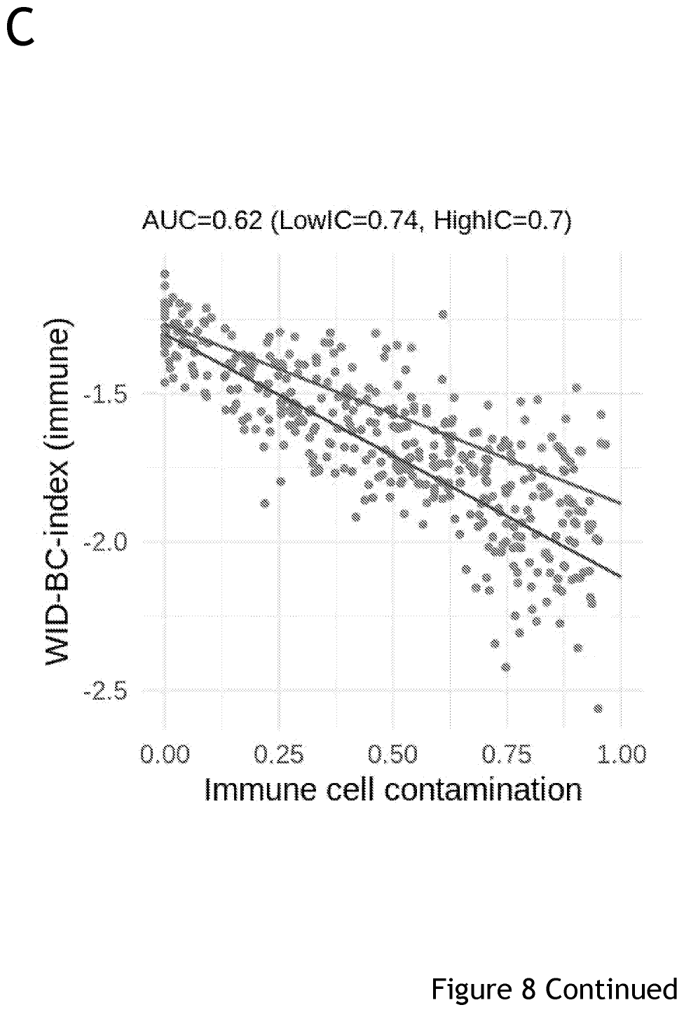

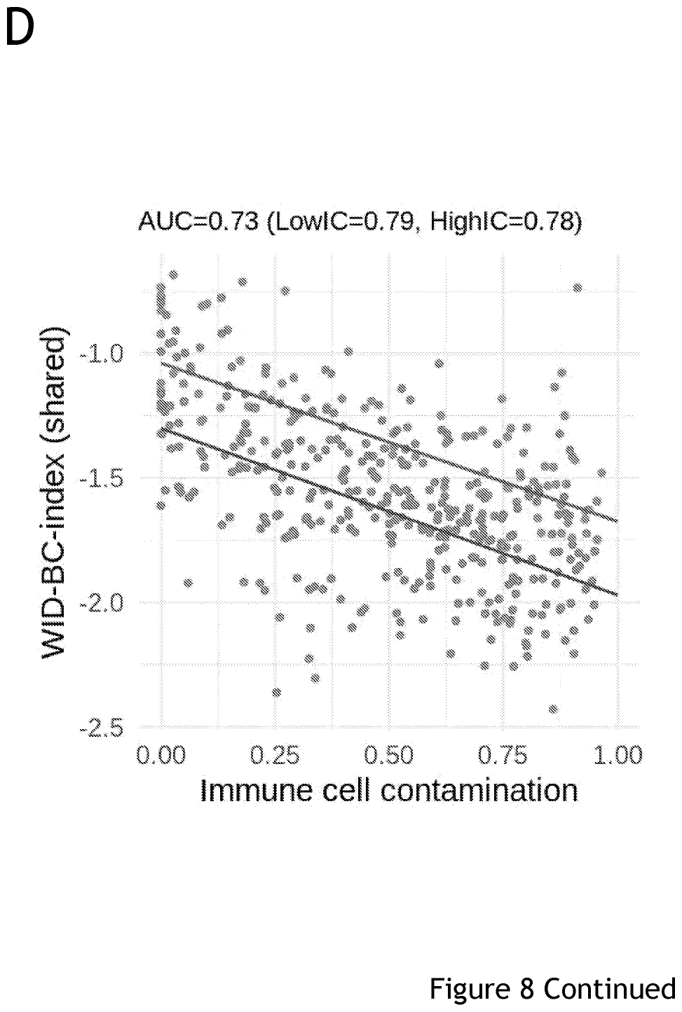

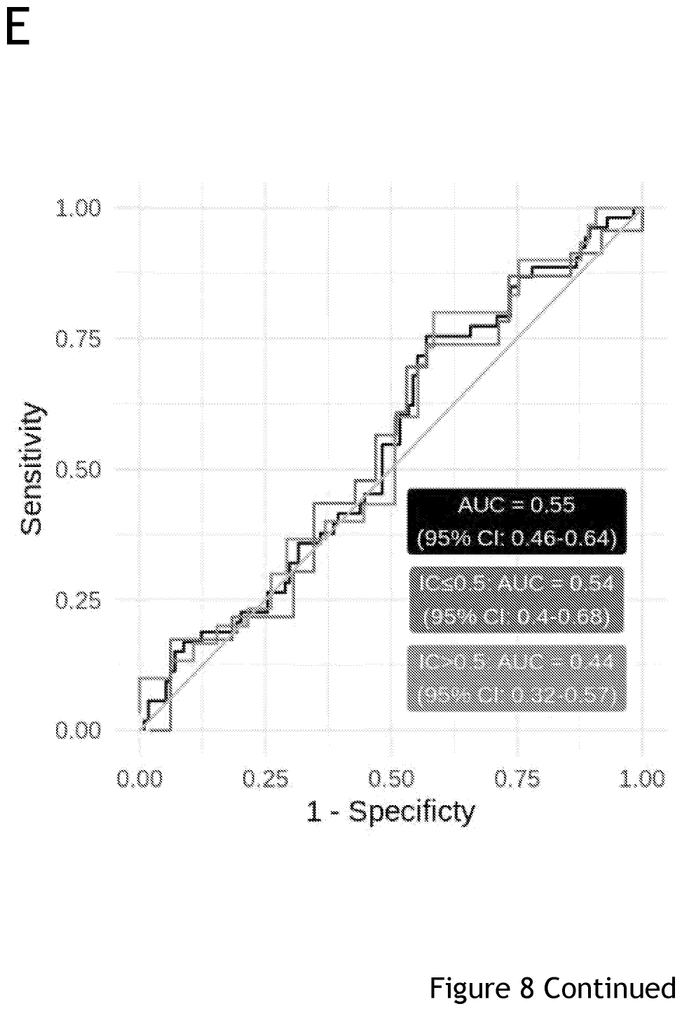

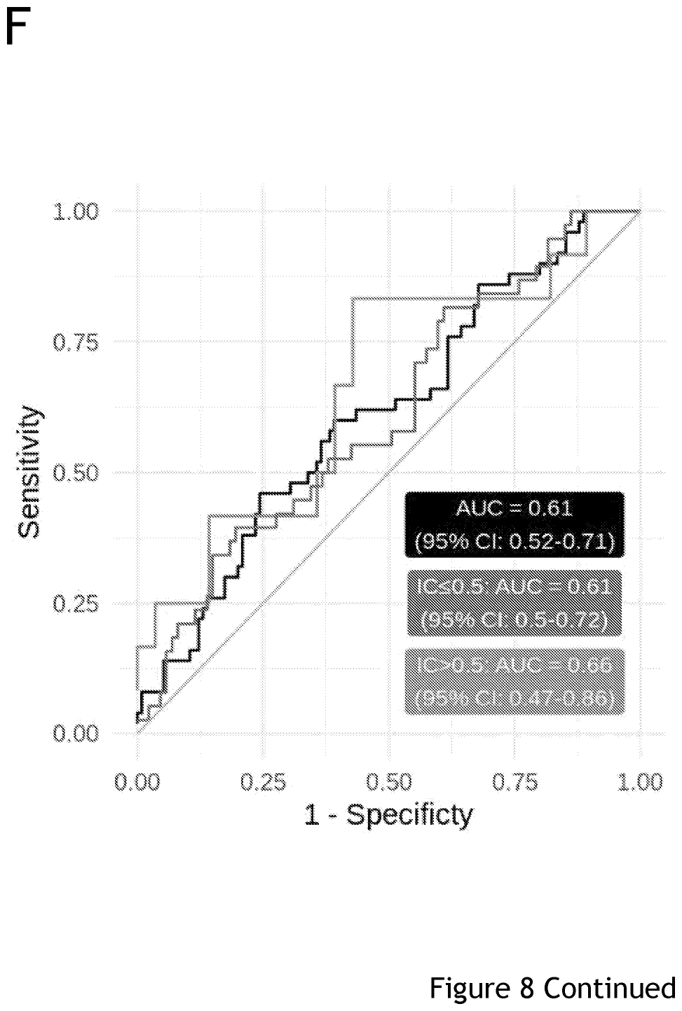

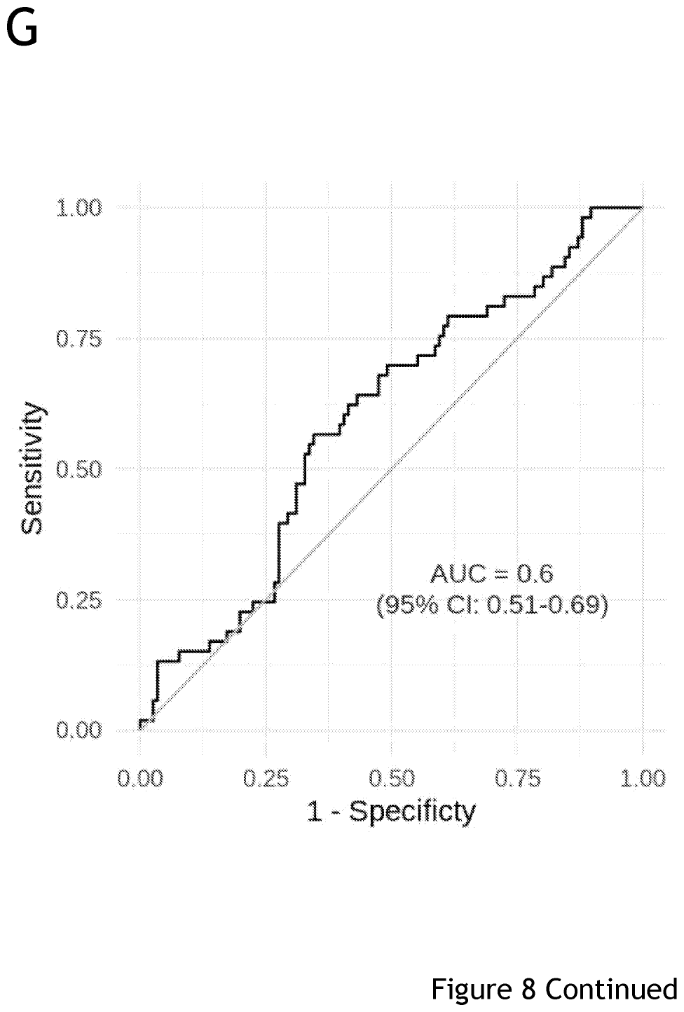

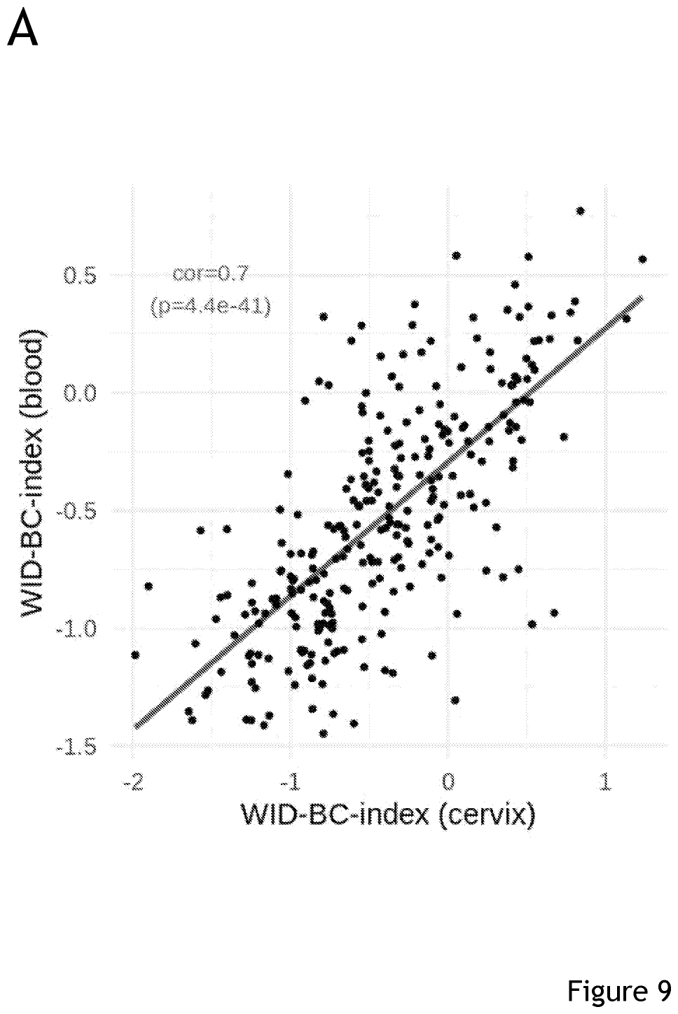

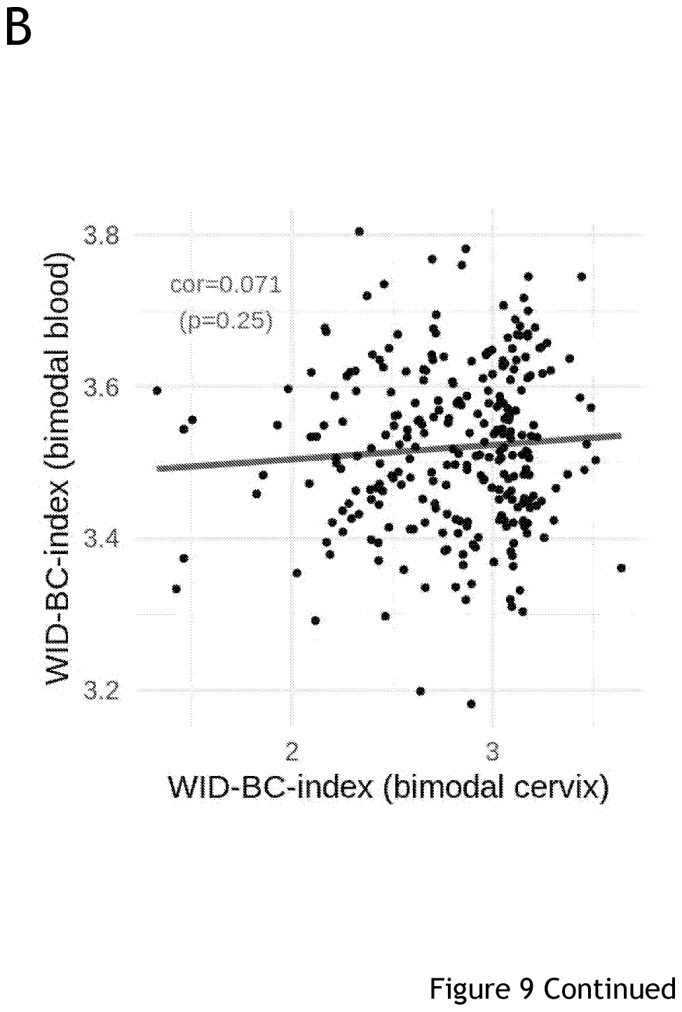

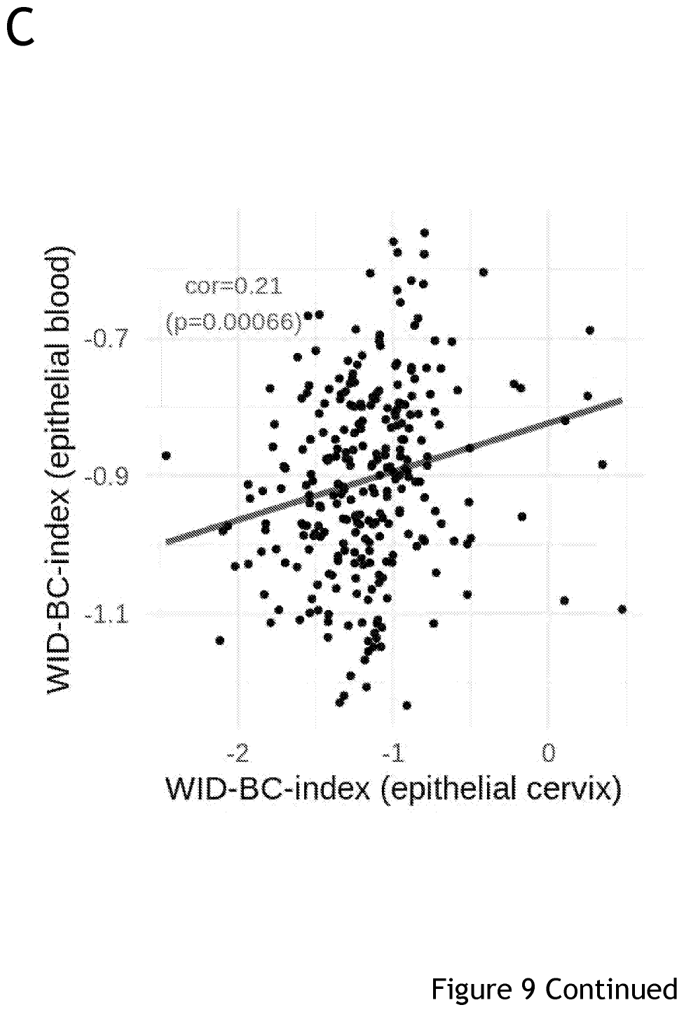

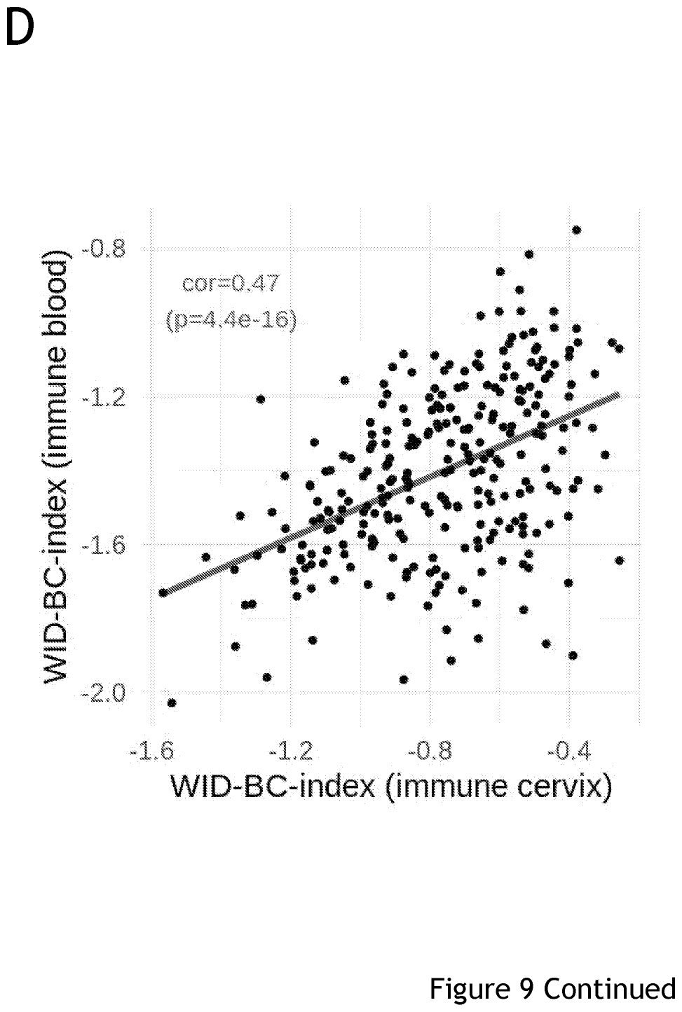

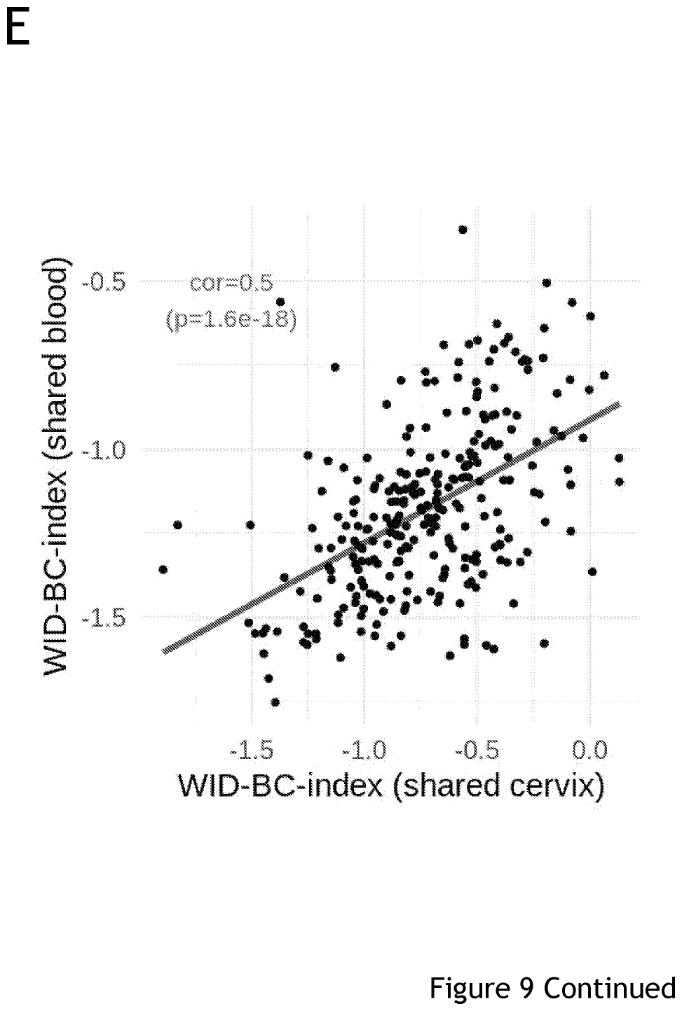

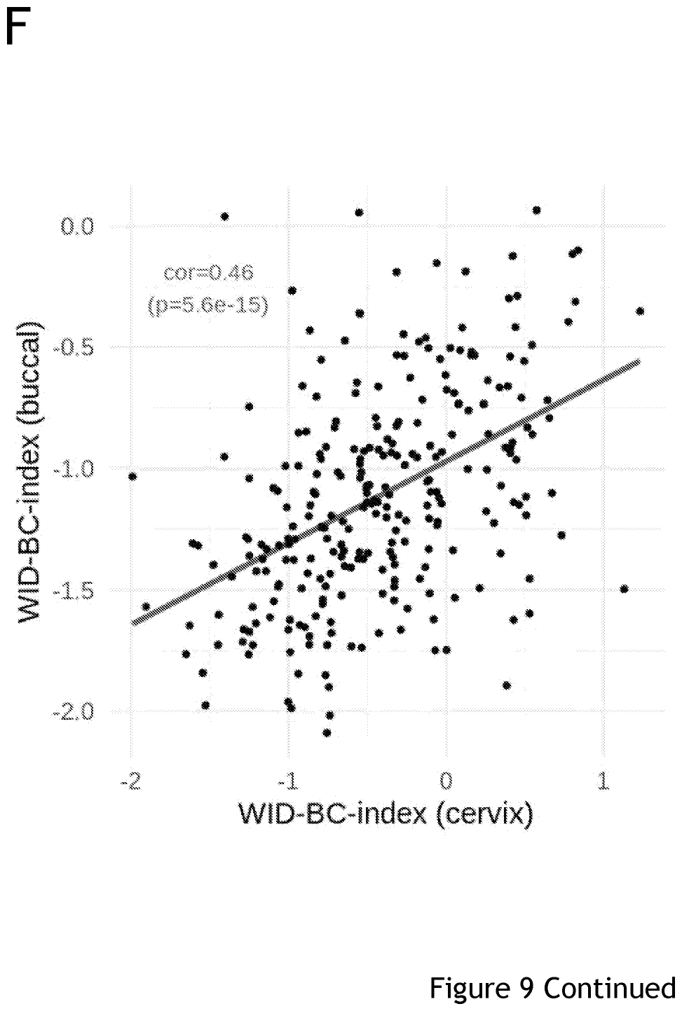

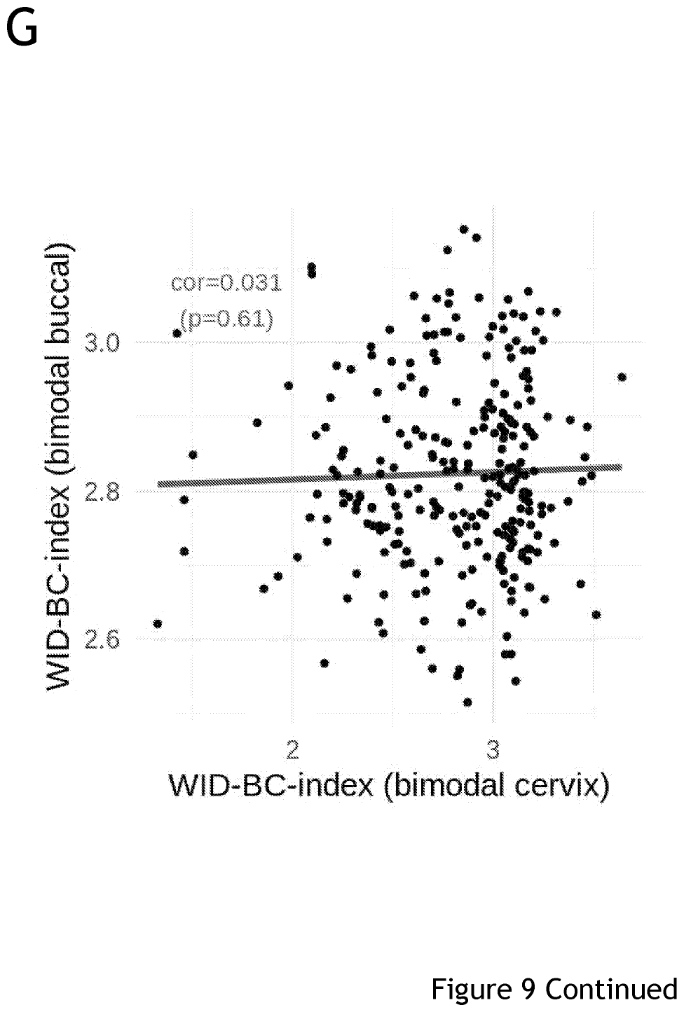

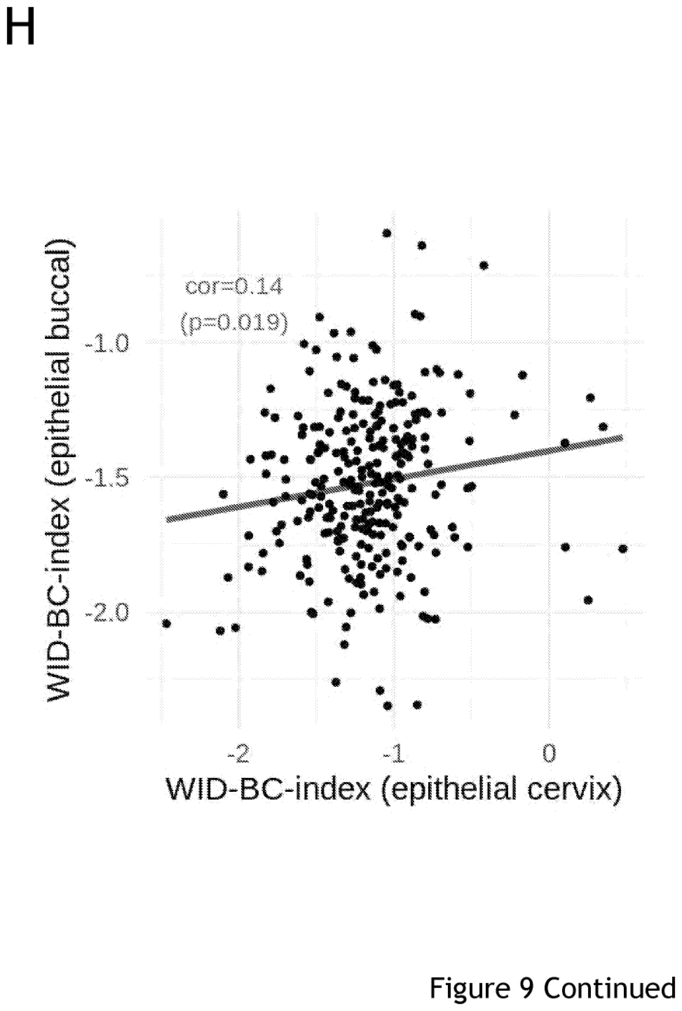

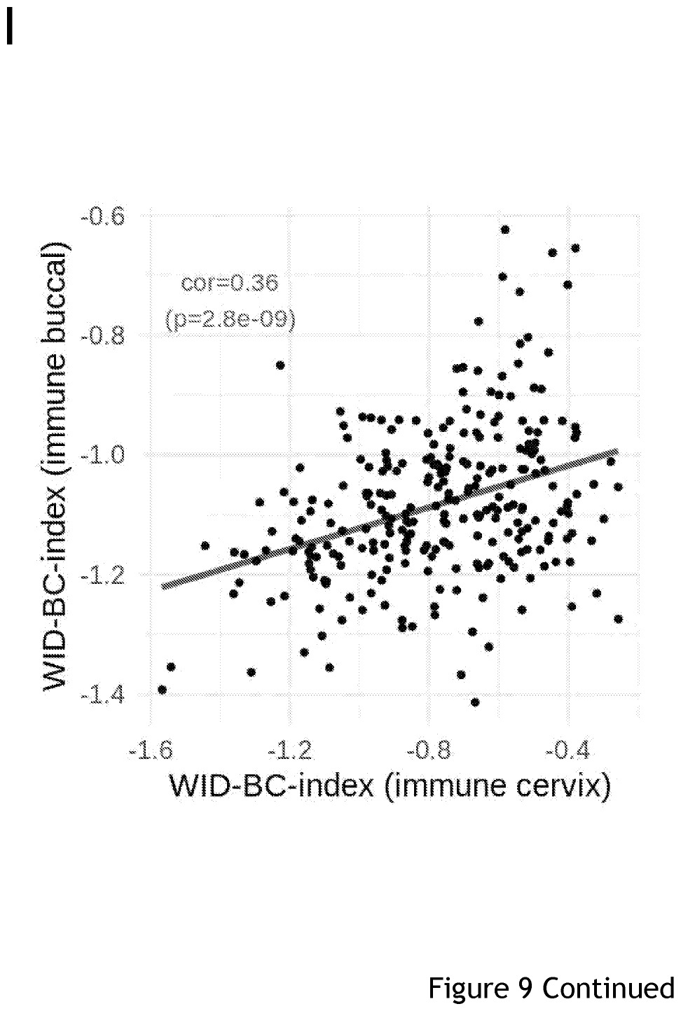

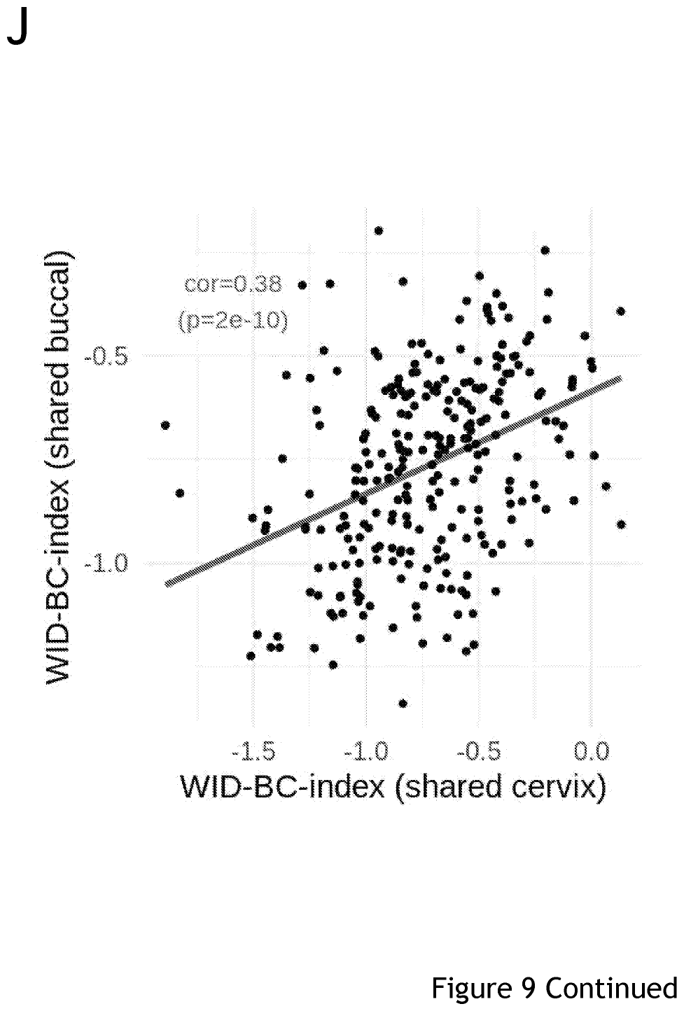

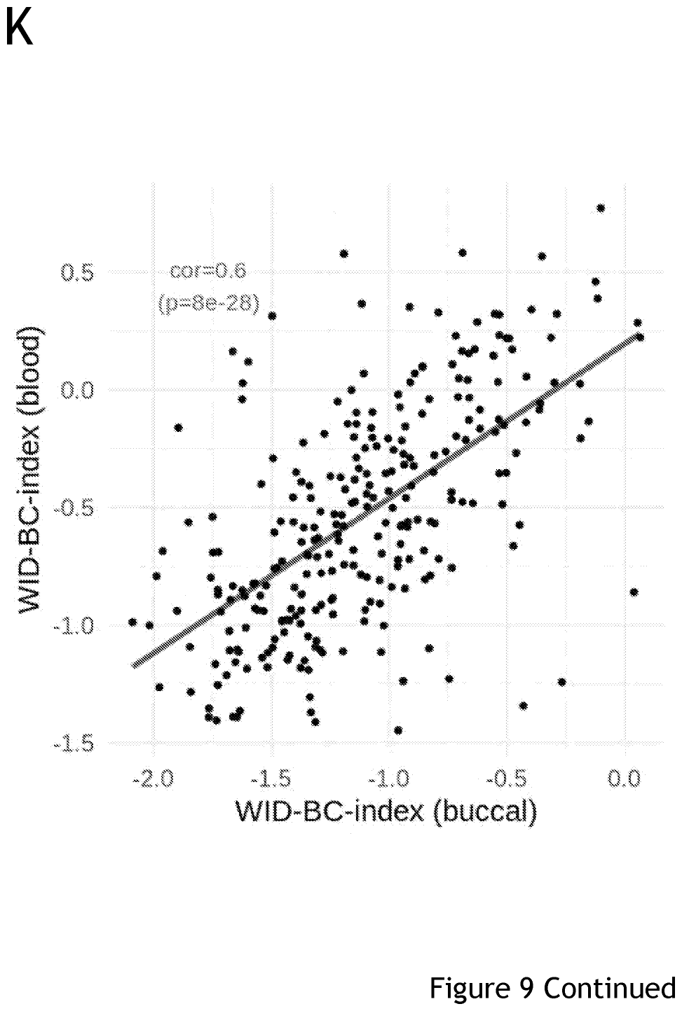

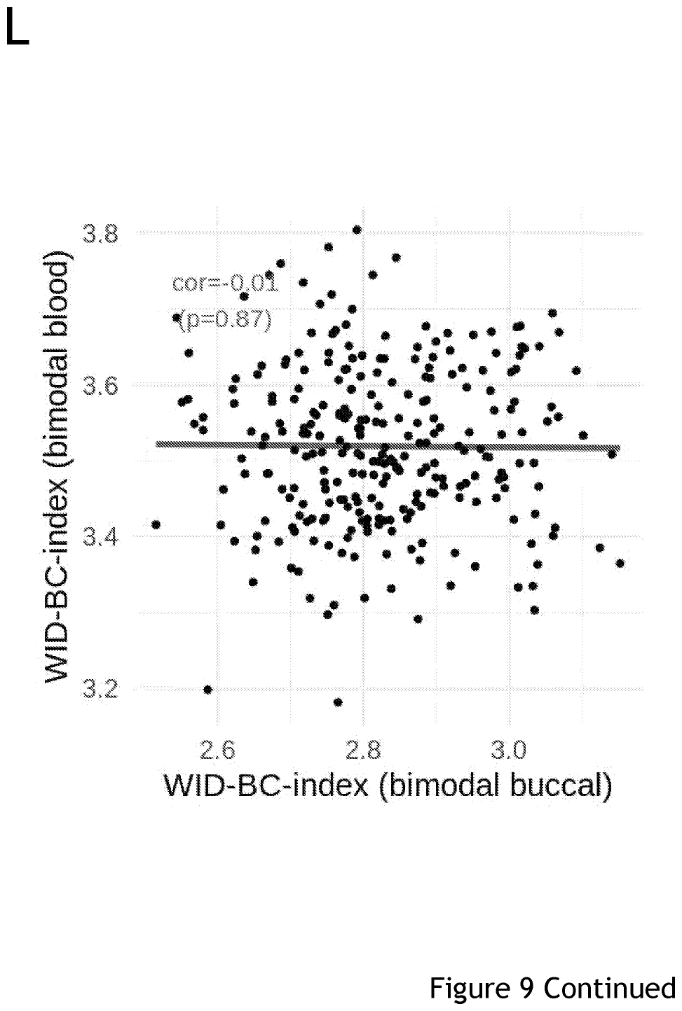

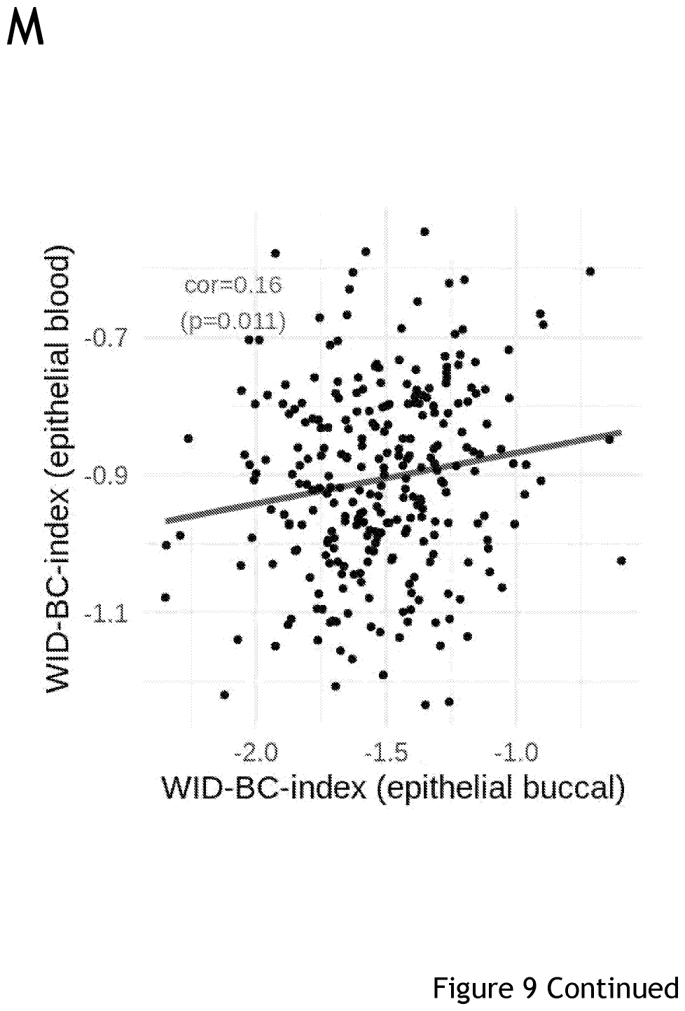

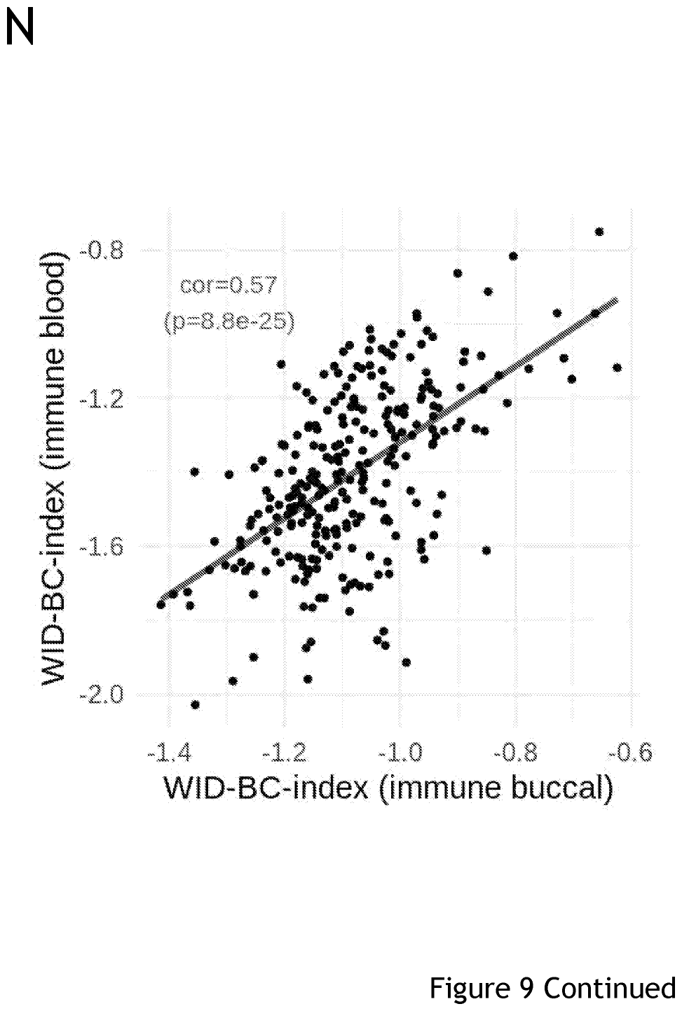

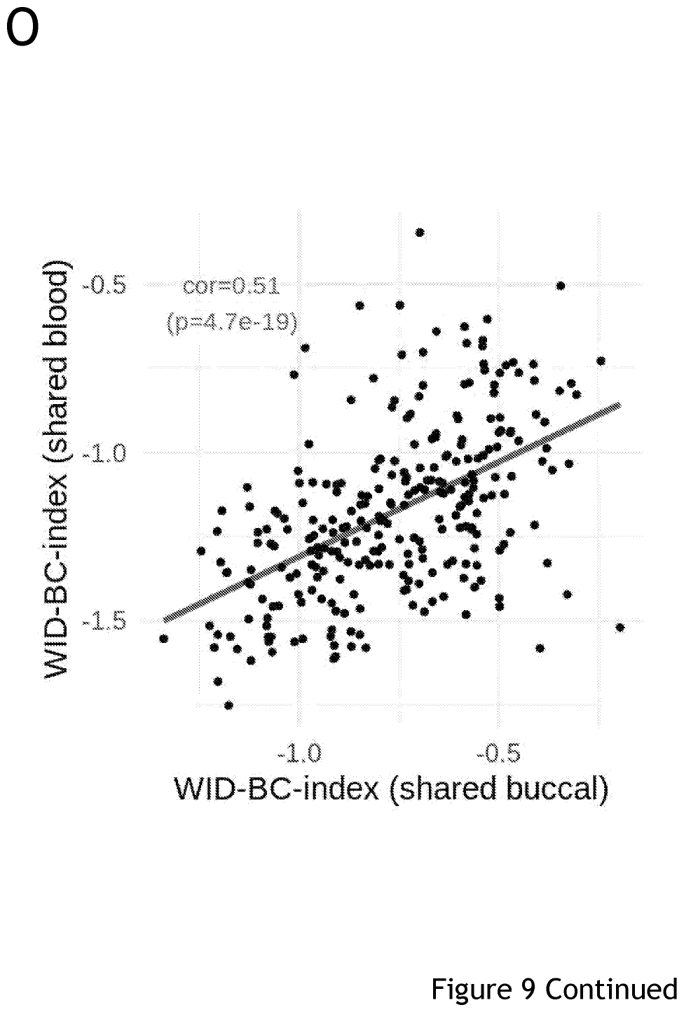

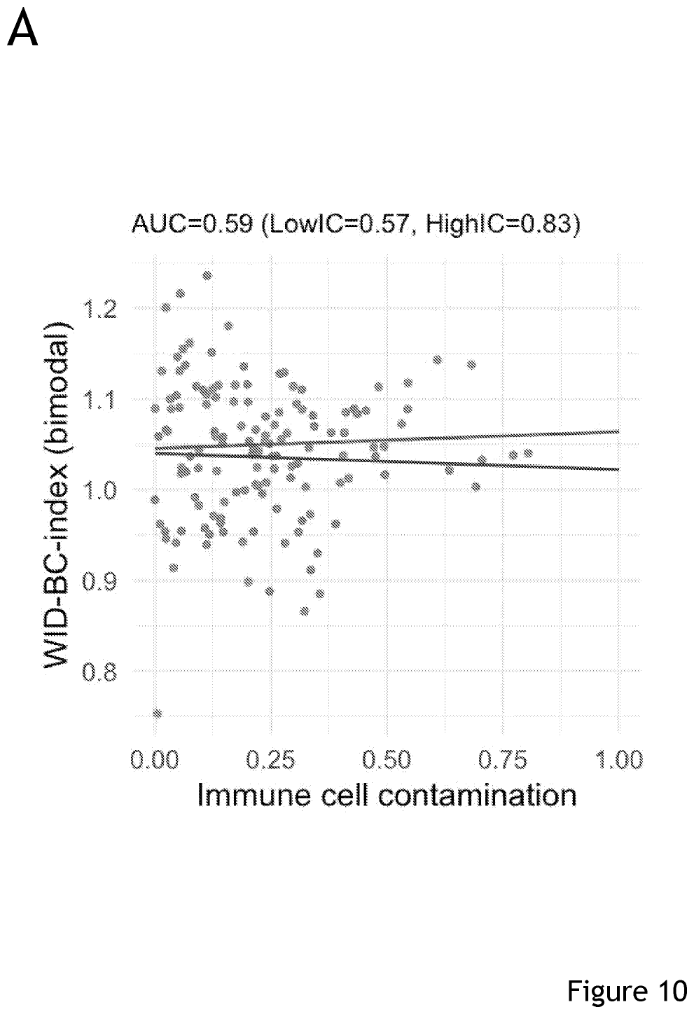

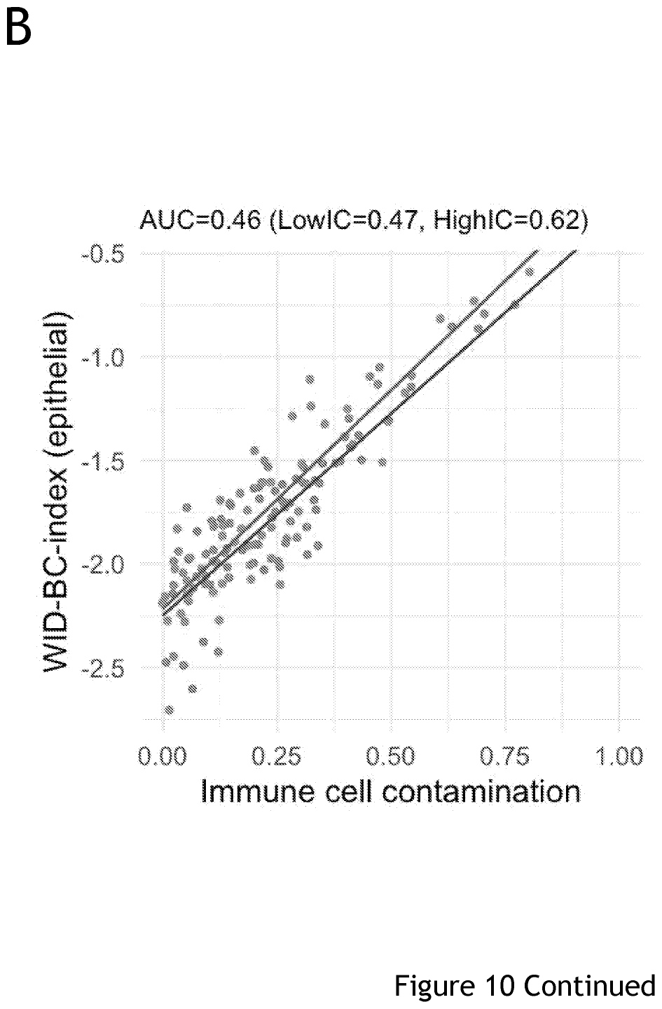

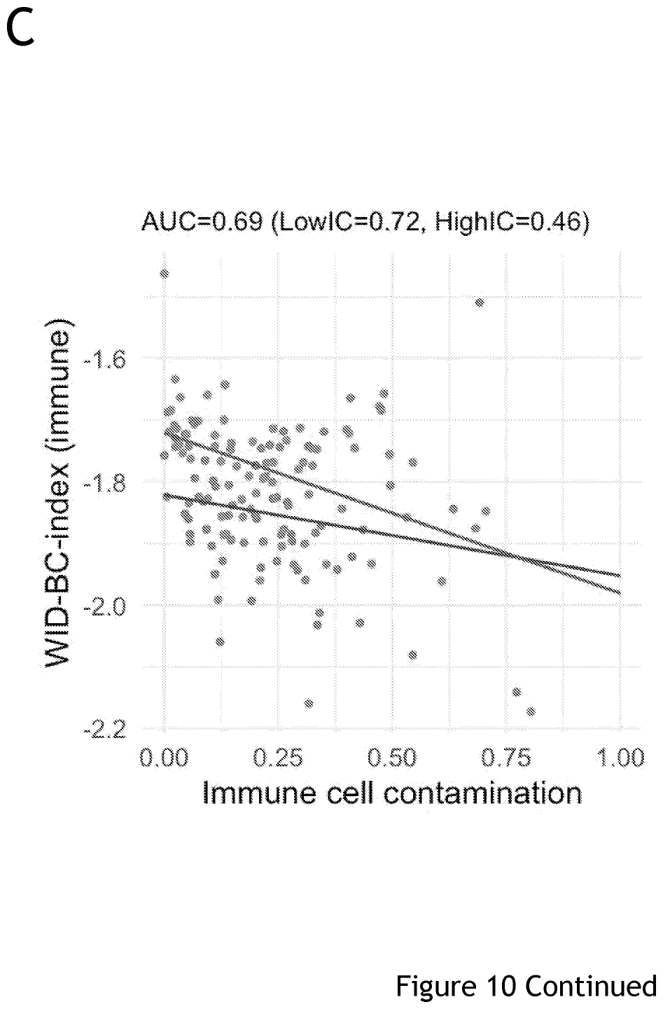

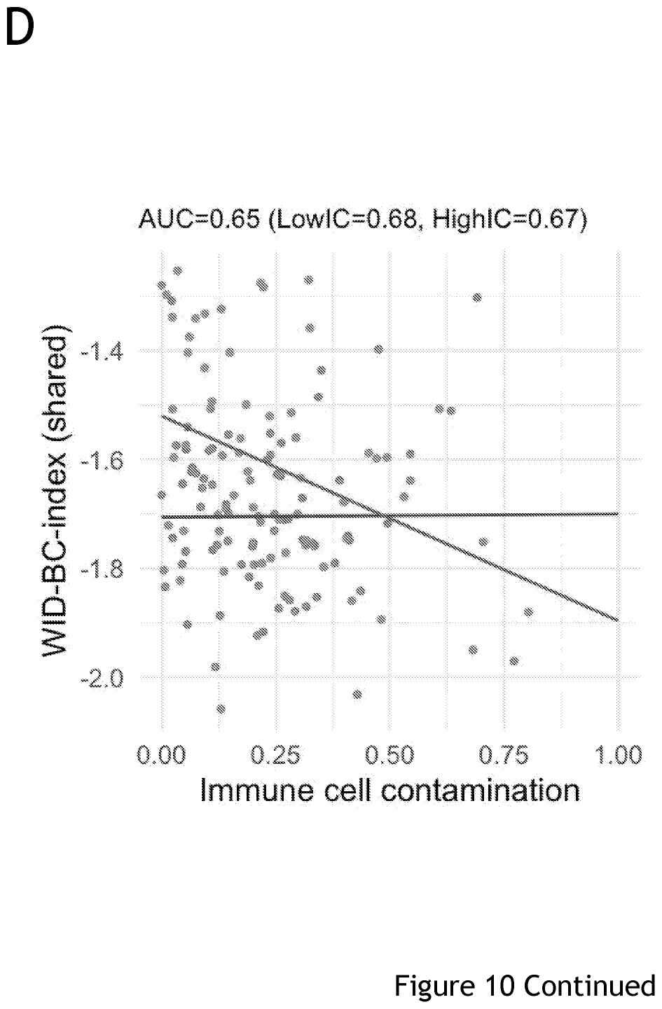

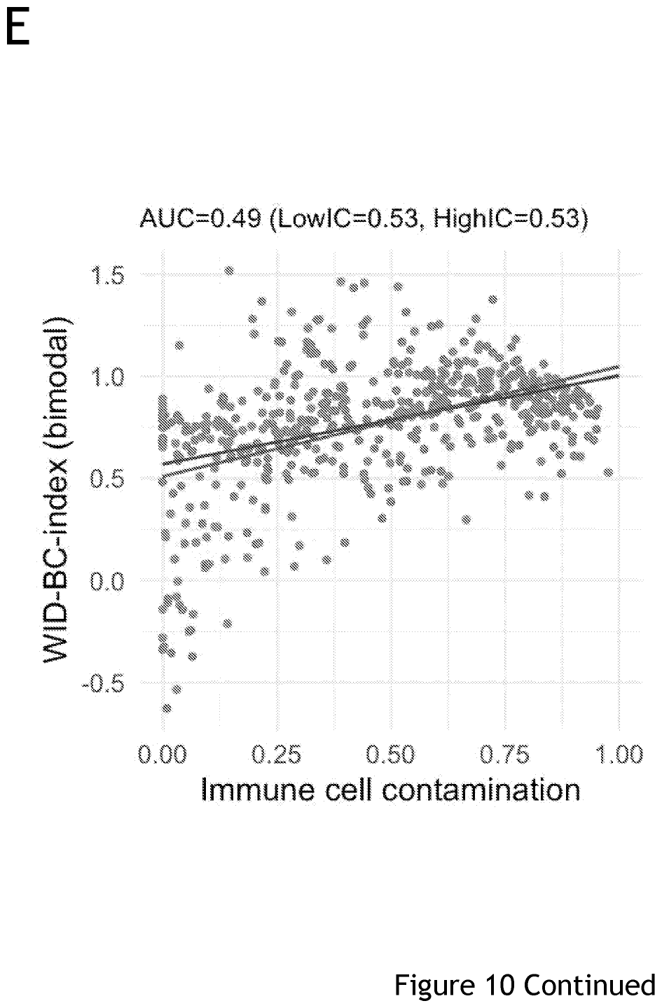

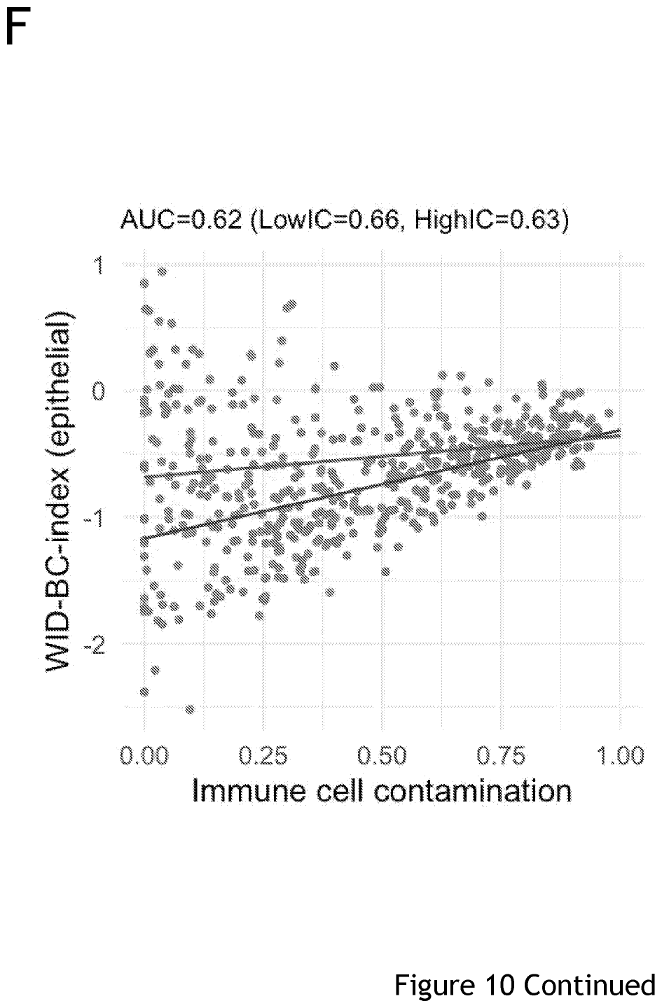

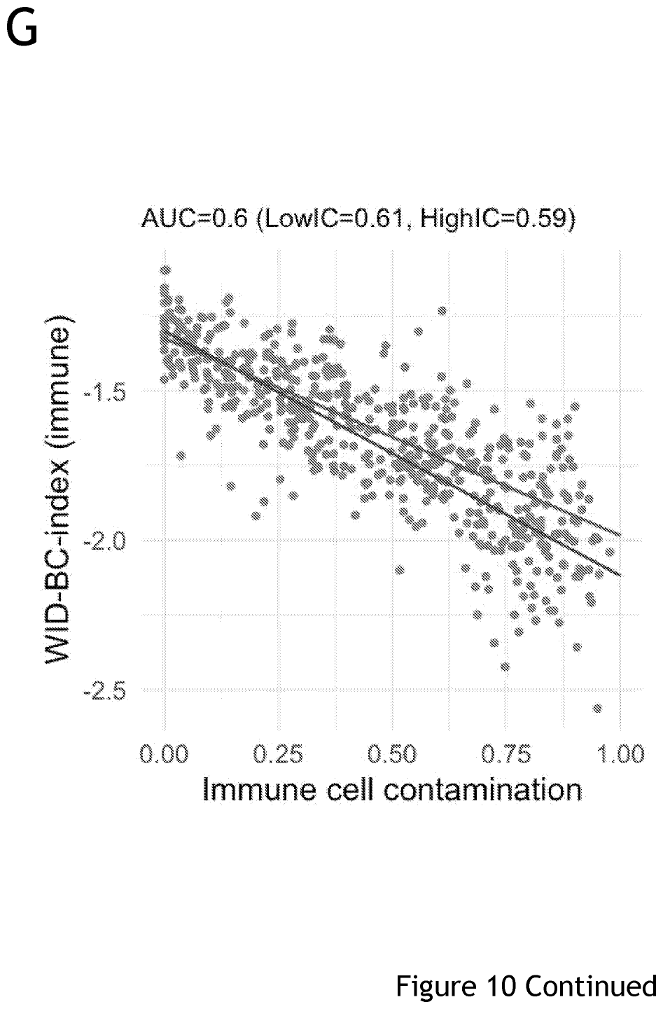

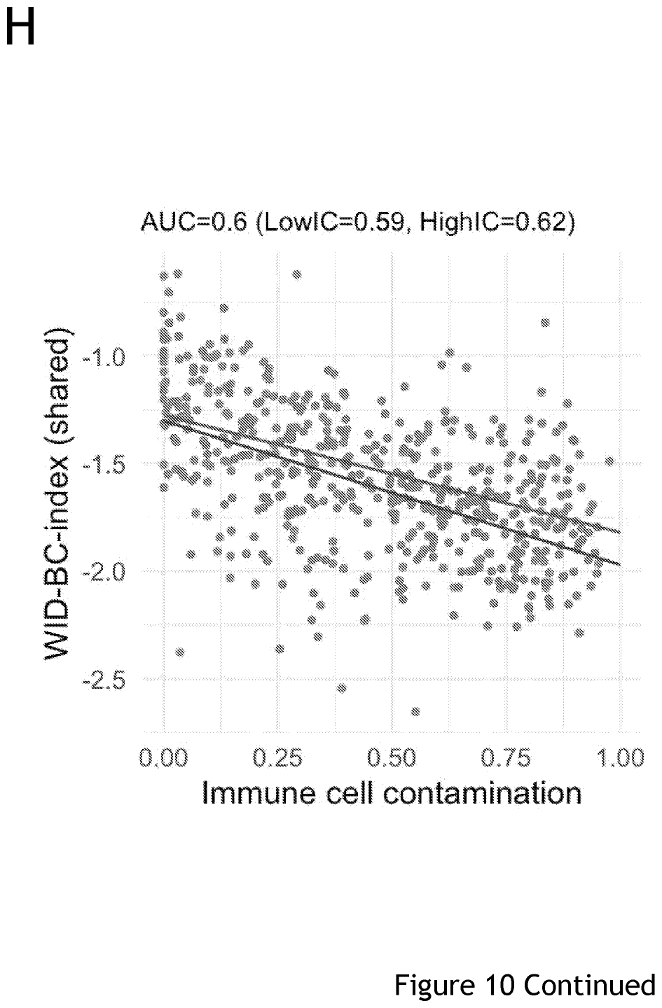

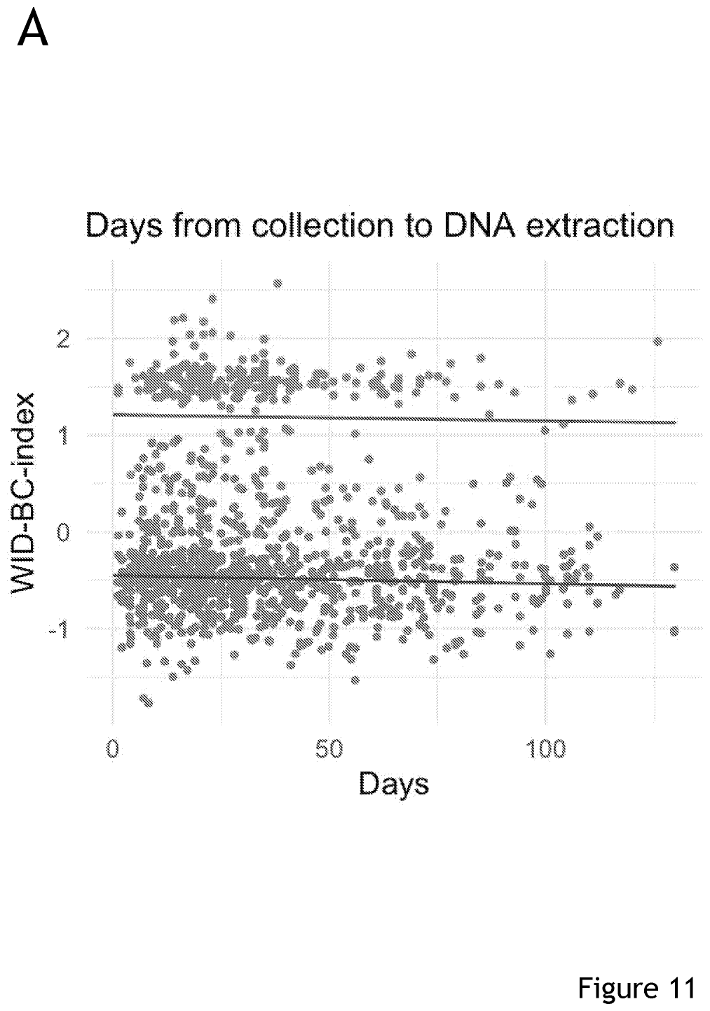

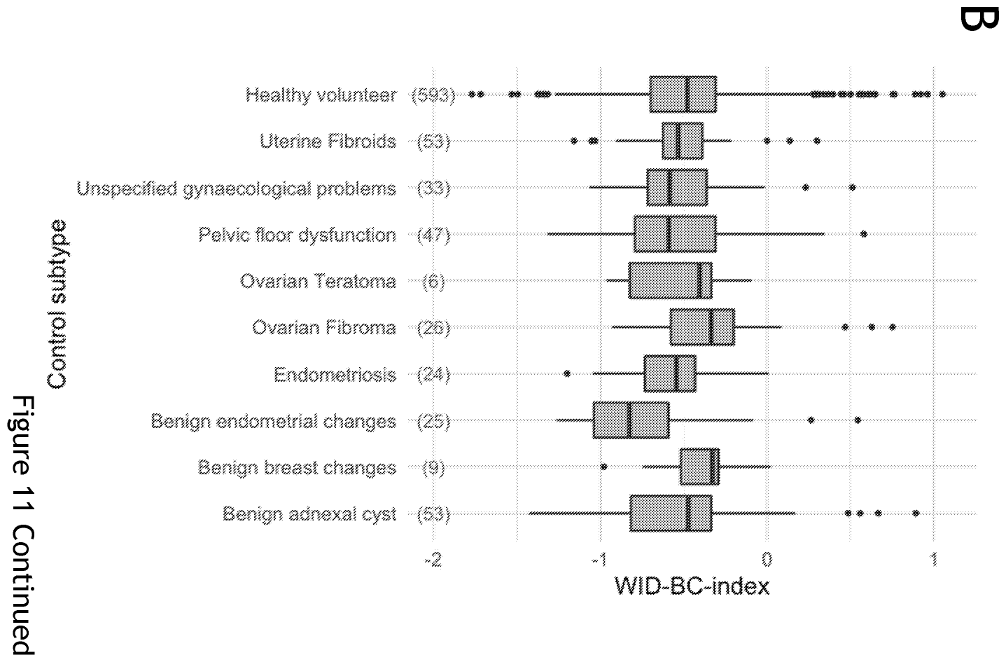

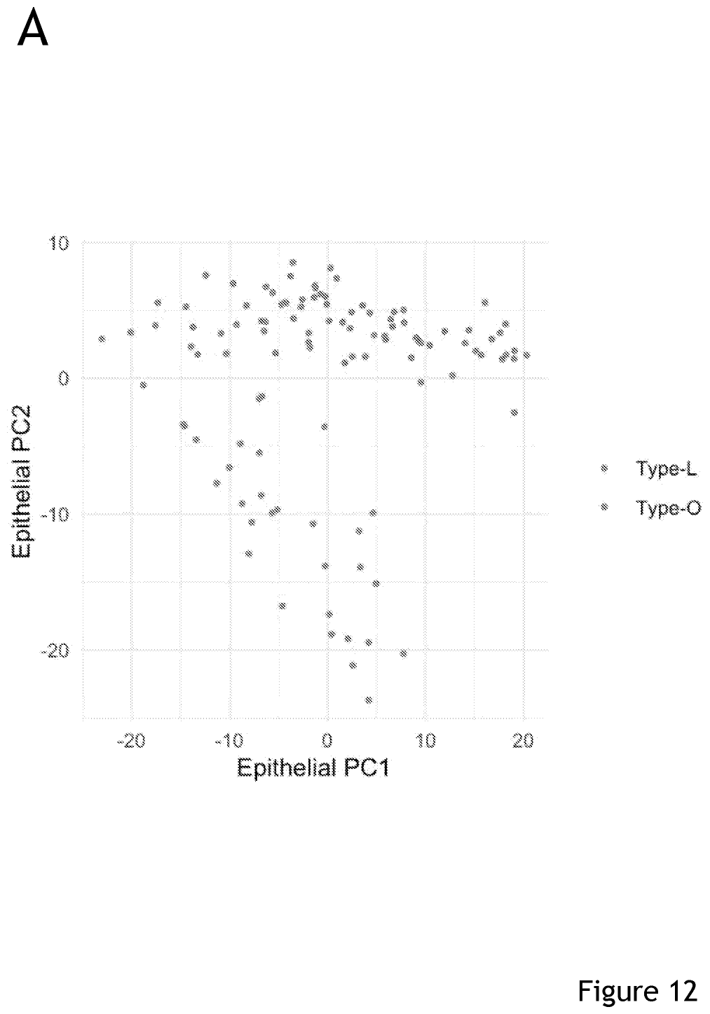

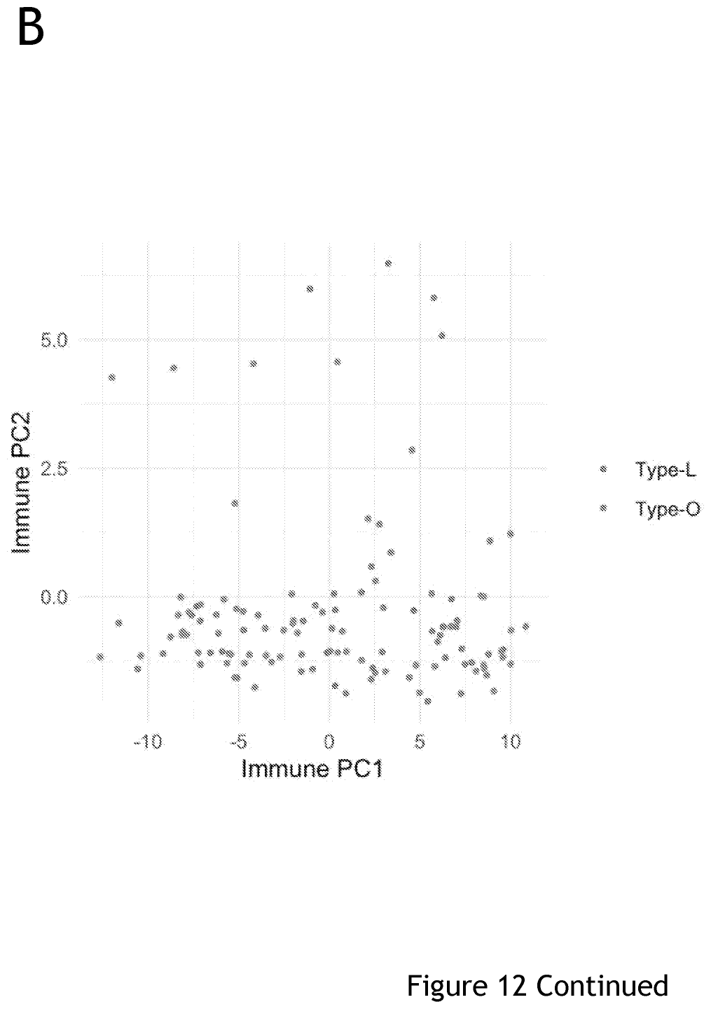

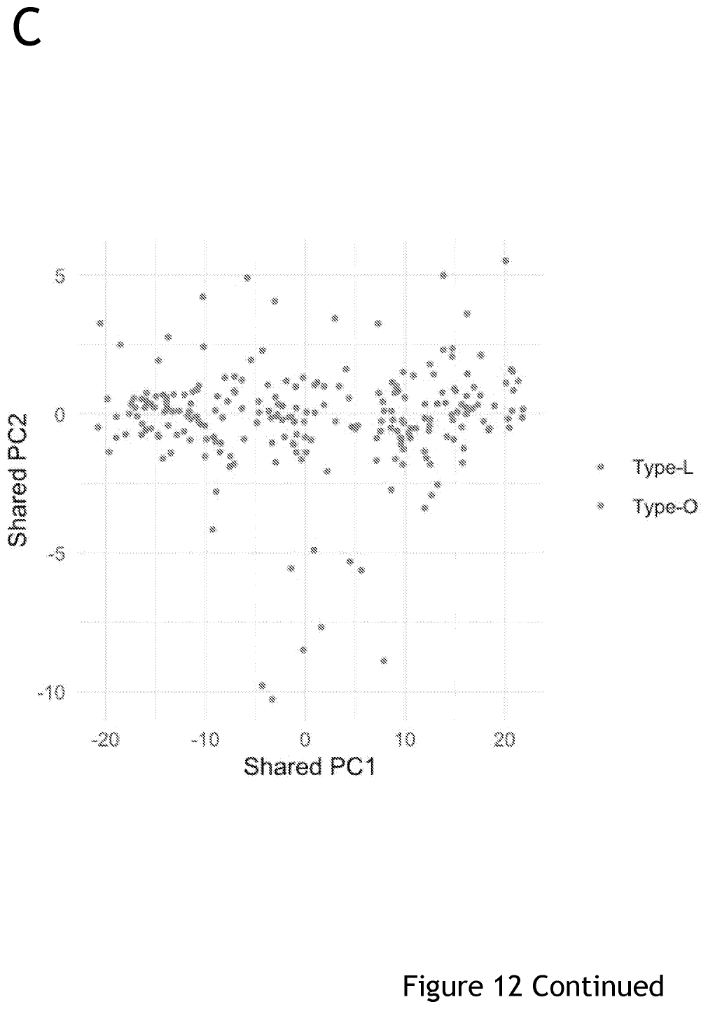

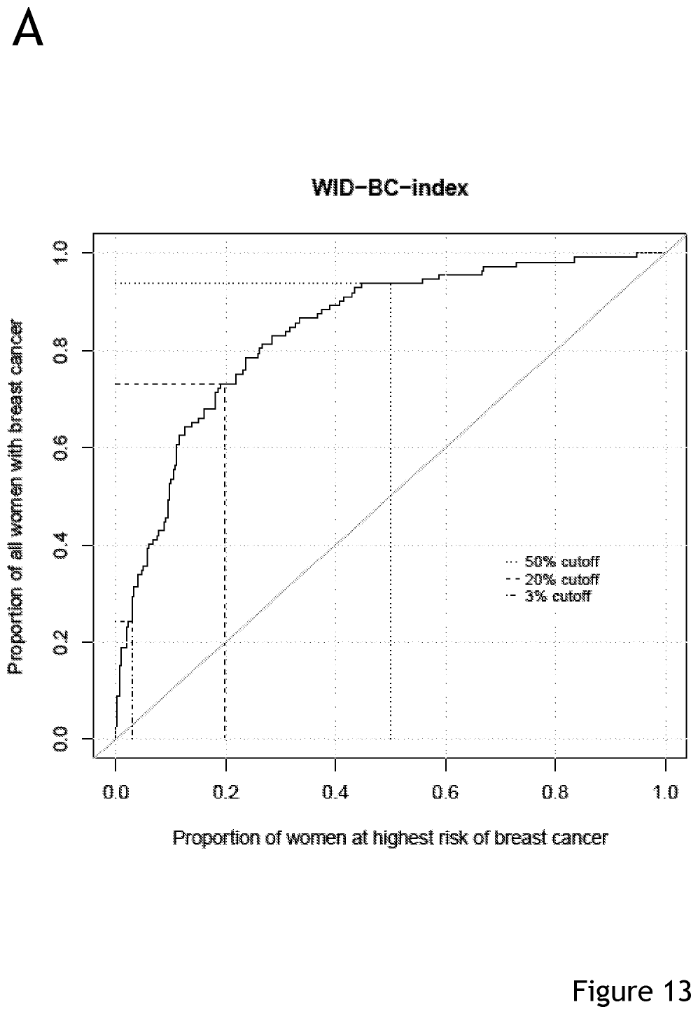

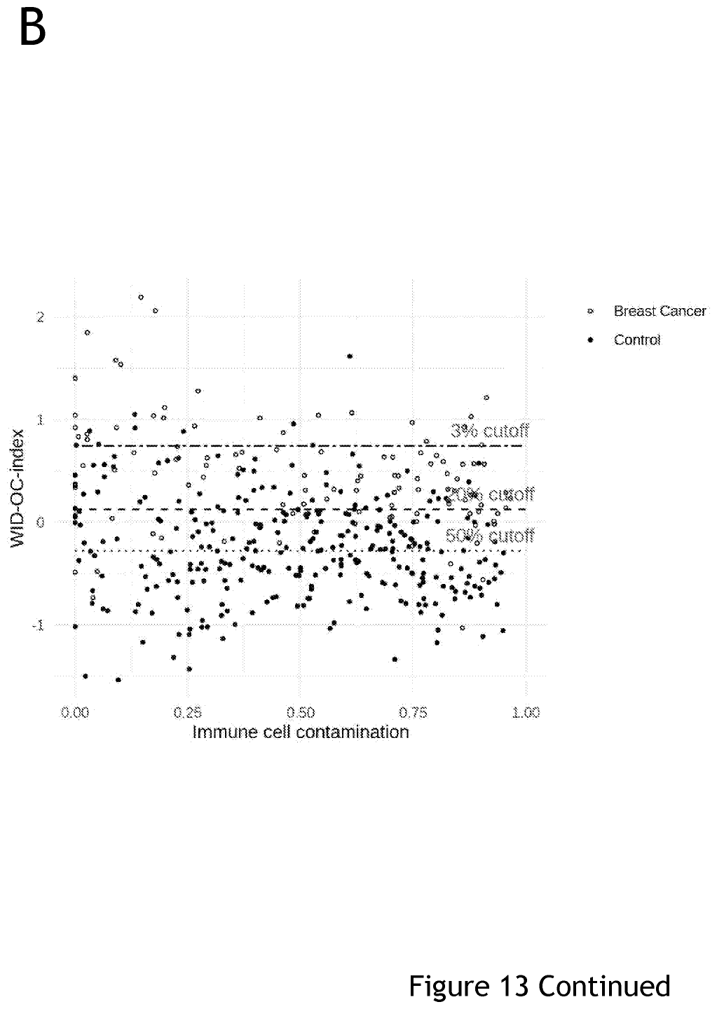

The inventors aimed to estimate how much variability across the 29,000 CpGs in the WID-BC-index could be attributed to epithelial cells or immune cells. An example of a CpG with high variability in epithelial cells and low variability in immune cells is given in FIG. 1B. For each CpG the inventors applied the following model. The inventors assumed that the epithelial beta values follow a beta distribution Beta(β|a₀,b₀) with shape parameters a₀>0 and b₀>0, and that immune beta values followed Beta(β|a₁, b₁) with shape parameters a₁>0 and b₁>0. The inventors assumed that each sample is a combination of epithelial and immune cells and that ρᵢ∈[0,1] is the proportion of immune cells in sample i, i=1, . . . , N. The quantities ρᵢ were obtained from the EpiDISH algorithm. The following log likelihood function was numerically optimised with respect to a₀, b₀, a₁, b₁:

$$L ( {a}_{0} , {b}_{0} , {a}_{1} , {b}_{1} ) = - \frac{1}{N} {\sum }_{i = 1}^{N} log [ ( 1 - {ρ}_{i} ) Beta ( {β}_{i} \vert {a}_{0} , {b}_{0} ) + {ρ}_{i} Beta ( {β}_{i} \vert {a}_{1} , {b}_{1} ) ]$$

and the variance of the epithelial and immune beta distributions were used as estimates of epithelial and immune variance.

### Enrichment Analyses

The epithelial delta-beta estimates were used to compute the top 1,000 hyper and hypo CpGs. These were used as inputs to the eFORGE 2.0 tool 24 (accessed at https://eforge.altiuisinstitute.org/). Data from the “Consolidated Roadmap Epigenomics DHS” were used for the analysis. The default options of 1 kb proximity window, 1,000 background repetitions, and strict and marginal significance thresholds of 0.01 and 0.05 were used.

A gene set enrichment analysis (GSEA) 25 was carried out by first selecting for each gene TSS200 region the CpG with the largest epithelial delta-beta estimate (both hyper- and hypo-methylated). Genes were then ranked according to the absolute value of these delta-beta estimates. The C2 curated gene set, c2.all.v6.2.symbols.gmt, was downloaded from MSigDB. The fgsea R package was used to perform the enrichment analysis with parameters minSize, maxSize, and nperm set to 15, 500, and 10,000 respectively.

### Analysis of Breast Tissue Samples

The cell type composition of each sample was estimated using EpIDISH with the epithelial, fibroblast, fat, and immune cell reference dataset. In contrast to cervical samples, fat cells constituted a substantial proportion of each sample. Results from cervical smear data indicated that the index performs independently of epithelial and immune proportions (fibroblasts formed a negligible proportion of cells). A linear adjustment was therefore made for fat content by splitting the samples into normal, BRCA carrier, adjacent, and TNBC groups. The inventors linearly regressed the WID-BC-index on fat in each group and obtained an estimate of what the index values would be if all four groups had the same fat composition. Similarly, samples from the mifepristone trial were split into mifepristone before, mifepristone after, placebo before, and placebo after groups. Within each group the inventors linearly regressed the index on fat proportion in order to obtain estimates of the index after adjustment for fat content.

### SNP Genotyping, QC and Imputation

In total, 318 breast cancer case subjects and 850 controls from the methylation discovery cohort were taken forward for genotyping using an Illumina 650 k Infinium Global Screening Array (GSA). Whole blood DNA was normalised to 75 ng/ul and a total of 300 ng applied to the Infinium Global Screening Array −24 V2 (Illumina, CA, USA) at UCL Genomics according to the manufacturer's standard protocol.

One control subject from this cohort failed to genotype. Genotype calling was performed using GenomeStudio, with genetic variants found to be clustering poorly being removed from further analyses. For duplicate genetic variant pairs, the variant within each pair with the lowest calling and clustering score was excluded. Autosomal SNPs were used in subsequent QC and PRS analyses (except for checks for sex mismatches, where the X chromosome was used to infer sex).

General subject and single nucleotide polymorphism (SNP) quality control (QC) was performed using PLINK version 1.9³¹. Three breast cancer cases and eight controls with a call rate less than 95% were excluded. One breast cancer case and three controls were further removed due to genetically inferred sex not being female. Genetic variants with a missing genotype rate greater than 5%, minor allele frequency (MAF) less than 1% or a significant departure from Hardy-Weinberg equilibrium (p-value <5×10⁻⁶) were excluded.

KING³², a relatedness inference algorithm, was used to identify duplicate/monozygotic twin or first-degree relative pairs. One control subject pair was identified as being a duplicate/monozygotic twin pair, and nine control pairs were inferred to be first-degree relatives. The subject within each related pair with the lowest call rate was excluded. After performing QC, 314 breast cancer case subjects, 816 controls and 479,105 variants were retained in the SNP discovery sample.

Non-European subjects were identified by plotting the top two principal components, generated using GCTA version 1.26.0, for the SNP discovery samples and 270 HapMap phase II release 23 samples (CEU, YRI, JPT and CHB individuals) downloaded in PLINK-formatted binary files. Subjects found not to cluster around HapMap European samples were excluded from further analyses. After excluding non-European subjects, 305 breast cancer cases and 754 controls were retained in the SNP discovery sample.

Using the Michigan Imputation Server 33 and 1000 Genomes Phase 3 reference panel, the SNP discovery dataset went through further QC before being phased (Eagle2) and imputed. Variants where strand, allele, genetic position or allele frequencies were not concordant with the 1000 Genomes Phase 3 reference panel were removed before phasing and imputation using Strand Tools.

After imputation, exclusion of variants with imputation R²<0.5 and removal of variants observed to have 3 or more alleles, 303 of the 313 SNPs used by Mavaddat et al. 22 to develop a 313 SNP breast cancer polygenic risk score (PRS) were successfully imputed. The inventors constructed a breast cancer PRS for each subject in the discovery cohort, such that the PRS is equal to:

$${PRS}_{j} = {\sum }_{i = 1}^{303} {\hat{β}}_{i} {x}_{ij}$$

where, \hat{β}ᵢ is the log odds ratio for the i-th SNP taken from publically available Oncoarray summary association results³⁴ (combined Oncoarray, iCOGs and BCAC overall breast cancer beta values) and xᵢⱼ is the number of copies of the effect allele present in each discovery cohort subject. Scores were generated using PLINK version 1.9.

### Data Availability

DNAme data has been deposited in the European Genome-phenome Archive (EGA), which is hosted by the EBI and the CRG, under accession number EGASXXXXXXXXXXX.

### EXAMPLE

The inventors performed an epigenome-wide DNAme analysis in cervical smear samples from women who had recently been diagnosed with breast cancer, and in matched controls, and established the WID-BC-index (Women's risk IDentification for Breast Cancer index) which the inventors validated in buccal samples and in an independent set of cervical samples. Further validation was done in samples from women with ovarian cancer and a set of matched cervical, buccal, and blood samples from BRCA mutation carriers. In addition, the inventors assessed the WID-BC-index in a larger number of different cell types as well as in normal breast and breast cancer tissue samples.

### Sample Heterogeneity and Differential Methylation

For the Discovery Set, the inventors collected samples from 329 women with primary breast cancers with poor prognosis features (defined by >2 cm cancers and/or lymph-node positive and/or grade 3 and/or hormone-receptor negative) from 14 European centres at the time of diagnosis and before treatment commenced, and 869 women without breast cancer (593 from the general population and 276 from women attending hospital for benign women-specific conditions) (Extended Data Table 1). Epigenome-wide DNAme was analysed using an Illumina Infinium EPIC bead chip array which encompasses over 850,000 CpG sites¹⁸.

The inventors assessed the level of cell type heterogeneity in each cervical smear sample using HEpiDISH¹⁹, an algorithm that infers the relative proportion of epithelial cells, fibroblasts, and seven subtypes of immune cells in each sample. The distribution of immune cell contamination (IC) was approximately uniform in both samples from cancer cases and controls. There was a significantly greater proportion of epithelial cells in cancers, and correspondingly fewer immune cells across all subtypes in the discovery dataset (FIG. 1A). This difference was comparatively small however, and absent in the external validation dataset (FIG. 7A).

Identifying CpGs with differential methylation between cases and controls was hampered by sample heterogeneity, since any differential methylation specific to epithelial cells was greatly diminished in samples with high IC (FIG. 1B). In order to infer which CpGs may contain a potential discriminatory signal, the inventors developed a statistical protocol to estimate the delta-beta (i.e. difference in mean proportion of methylated cells) between cases and controls in a pure epithelial cell sample, and a pure immune cell sample. The inventors linearly regressed beta values on IC fraction in both cases and controls separately. The difference between the two points where these lines intercept the y-axis at IC=0, gives an estimate of the delta-beta between cases and controls in pure epithelial cells. Conversely, the difference between intercept points at the IC=1 axis gives a delta-beta estimate in immune cells.

### Development of Discriminatory Index

In order to derive a diagnostic methylation signature, termed the WID-BC-index, the inventors used ridge and lasso regression to classify individuals as cases or controls. Classifiers were trained on two thirds of the discovery dataset (572 cancer-free controls, 217 breast cancer cases) and the remaining one third was used as an internal validation set (297 controls, 112 cases) with the intention of evaluating their performance as a function of the number of CpGs used to construct the index. The area under the receiver operator characteristic curve (AUC) was used as a measure of predictive performance. CpGs were ranked by combining the epithelial delta-beta ranking with the immune delta-beta ranking.

Predictive performance was evaluated as a function of n, the number of CpGs used to train the classifier, using the internal validation dataset (FIG. 1C) and optimal performance of 0.84 (95% CI: 0.80-0.88) was achieved using 29,000 CpGs with ridge regression (FIG. 1D). The WID-BC-index was moderately, but significantly associated with IC fraction in the internal validation set (FIG. 1E, linear regression coefficients of −0.55, p=0.004 and −0.20, p=0.07 in cases and controls, respectively).

In samples with an IC fraction ≤0.5 and IC>0.5 the AUC was 0.85 (95% CI: 0.78-0.91) and 0.86 (95% CI: 0.80-0.91), respectively, suggesting that discriminatory signals are present in both epithelial and immune cells. In order to resolve cell-type specific signals the inventors attempted to infer which of the 29,000 CpGs account for most of the variability in epithelial cells (and hence potentially drive an epithelial-specific signal) and which vary most in immune cells. Firstly, it was observed that a substantial number of CpGs follow the distinctive bimodal distribution in epithelial cells as shown in FIG. 1B. The inventors developed a bimodality detection algorithm and found that 8,442 out of the 29,000 CpGs followed this pattern. A principal component analysis of these bimodal CpGs in training set samples with an IC<0.5 revealed two distinct subgroups. In the internal validation set, 37.3% of the larger subgroup were breast cancer cases compared to 19.5% in the smaller subgroup (OR: 2.43; 95% CI: 1.23-4.84).

The inventors then developed a statistical model to infer the epithelial and immune variance at each remaining CpG site. CpGs were divided into “epithelial” (10,119 CpGs), “immune” (3,525 CpGs) or “shared” (6,914 CpGs) subsets as shown in FIG. 1F. Since the WID-BC-index is defined as a weighted sum of 29,000 beta-values the index can be split into four subcomponents by taking the weighted sums corresponding to each of the four subsets of CpGs (FIG. 1G). As expected, the epithelial subcomponent captures an epithelial-specific signal in the internal validation set, the immune subcomponent corresponds to an immune-specific signal, whereas the shared subcomponent captures a signal that is shared across both cell types (FIG. 8). The inventors evaluated the AUC in the internal validation set after omitting each subcomponent and observed that the removal of the epithelial component leads to the greatest reduction in performance suggesting that this component is particularly informative (FIG. 1H).

The inventors found that the index was highly depleted of CpG islands and enriched for Open Sea regions (FIG. 1I). Ridge regression combines information from all input CpGs in contrast to lasso regression, which typically selects a small subset of inputs. Ridge regression offered consistently superior performance suggesting that the discriminatory signal is most robustly extracted by combining a large number of comparatively weak signals from predominantly open sea CpG sites. The inventors ranked the 29,000 CpGs used to define the WID-BC-index according to the absolute value of the regression coefficients from the ridge model. In order to assess how informative the top CpG sites are the inventors trained sub-classifiers on the top n sites (FIG. 1J). The inventors observed that AUCs of 0.78 and 0.81 can be achieved with the top 500 and 1,000 CpGs respectively indicating that the top ranked subsets are particularly informative. The inventors also trained sub-classifiers after removing the top n CpGs, and on subsets of 500 CpGs after partitioning the ranked list into bins of size 500. In both cases, the inventors found that a substantial predictive signal is present.

### External Validation

A separate independent external validation dataset consisting of 225 controls and 113 cases was used to validate the index performance (Extended Data Table 1). The WID-BC-index was computed for each woman (FIG. 2A) resulting in an AUC of 0.81 (FIG. 2B; 95% CI: 0.76-0.86). There was no significant dependence on IC.

Validation in Women with a BRCA Mutation

DNAme is tissue specific and specific exposures are recorded in certain cell subtypes^{17,20,21}. The index decomposition in FIG. 1F implies that the WID-BC-index subtypes uses epithelial- and immune-specific signals as well as a shared component. In order to assess whether the WID-BC-index (derived from cervical smear samples) can be extended to other tissue types the inventors analysed an independent dataset of matched cervical, buccal and blood samples from yet unaffected BRCA1 (n=58) and BRCA2 (n=53) mutation carriers and 116 healthy controls. Similar to the cervical smears, a substantial proportion of buccal DNA originates from immune cells (Extended Data FIG. 2B,C). In BRCA1 carriers the inventors observed an AUC of 0.61 (FIG. 2C; 95% CI: 0.52-0.69) in cervical samples, 0.67 (FIG. 2D; 95% CI: 0.59-0.76) in buccal samples, and 0.64 (FIG. 2E; 95% CI: 0.56-0.72) in blood. In BRCA2 carriers whose breast cancer risk is lower the AUCs were 0.55 (95% CI: 0.52-0.69) in cervical samples, 0.61 (95% CI: 0.52-0.71) in buccal samples, and 0.60 (95% CI: 0.51-0.69) in blood (FIG. 8E-G).

Furthermore in this set, the inventors observed strong correlations between all three matched tissue types (FIG. 9). As expected, the strongest correlations were between the shared subcomponent confirming that this signal is not tied to any particular cell subtype. The immune subcomponent was also strongly correlated across tissues. The epithelial component was relatively weakly correlated, particularly between cervical and buccal samples.

### Performance of Index in Matched Buccal Samples

In order to further assess whether the WID-BC-index can discriminate breast cancer cases from unaffected controls based on DNAme profiles in buccal samples the inventors analysed matched buccal samples from a subset of 135 women in the internal validation set (69 controls and 66 cases). The cell type composition of these buccal samples was comparable to the BRCA dataset (FIG. 7G). The inventors found that the discriminatory signal derived using cervical smear samples was also present in these matched buccal samples (FIG. 3A), yielding an AUC of 0.69 (FIG. 3B; 95% CI: 0.60-0.79). There was a correlation of 0.57 (p<10-12) between the WID-BC-index computed in matched cervical and buccal samples. The immune and shared subcomponents showed the greatest discriminatory performance (FIG. 10C,D).

### Ovarian Cancer

The inventors additionally analysed 242 cervical samples from women with ovarian cancer. The inventors obtained an AUC of 0.69 (FIG. 3C,D; CI: 0.65-0.74) in these 242 samples and the same 297 control samples from the breast cancer internal validation set. The subcomponents followed a similar pattern as the breast cancer cases (FIG. 10E-H). Consistent with the breast cancer internal validation set, the odds ratio associated with the largest bimodal group was 1.6 (95% CI: 0.97-2.61).

For each of our validation datasets the inventors computed odds ratios corresponding to quartiles of the WID-BC-index (Tables 5A-F).

Association with Epidemiological and Clinical Factors

The inventors investigated the relationship between the WID-BC-index and various epidemiological variables using the internal and external validation datasets (the training dataset is not suitable because it was used to develop the index). A statistically significant association was found between the WID-BC-index and age (FIG. 4A, correlation coefficients of 0.27, p<10-9 and 0.33, p<10-6 in controls and cases respectively). The Illumina 650 k Infinium Global Screening Array was used to genotype matched blood samples from a subset of 314 cases and 816 controls in the discovery set. The inventors computed a recently published polygenic risk score (PRS; 303 of the 313 SNPs described 22 were used) for breast cancer prediction. In the 107 cases and 280 controls from the internal validation set the inventors found a modest but significant correlation of 0.13 (p=0.03) between the PRS and the WID-BC-index (FIG. 4B) in controls and no significant correlation in cases (correlation coefficient −0.03, p=0.7). In the entire discovery set the PRS had an AUC of 0.67 (FIG. 4C; 95% CI: 0.64-0.71). No significant association was found between the WID-BC-index and BMI (FIG. 4D). No significant difference in the WID-BC-index was observed between individuals with 0 and >1 first-degree-relatives with breast cancer (FIG. 4E), age of menarche (FIG. 4F), or age at first live birth (FIG. 4G). The index was significantly higher in postmenopausal women (FIG. 4H, p<10-5) and women who had undergone hormone replacement therapy (FIG. 4I, p<0.001). There was no association between the WID-BC-index and clinic-pathological features of the cancers.

No significant association was found between the WID-BC-index and various technical parameters including the time between sample collection and processing (FIG. 11A), date of processing, plate number (samples were processed on 96 sample plates) and sentrix position. No difference was found between control samples from healthy volunteers and women presenting at hospitals for benign women-specific conditions (FIG. 11B).

### The WID-BC-Index is Reflective of a Fat-Cell Differentiation

In order to assess whether the WID-BC-index is reflective of a cell-specific program the inventors analysed all ENCODE samples (Additional Data) for which EPIC array data were available. The inventors ranked and plotted the WID-BC-index in all primary cell samples and in vitro differentiated cell samples and found a high WID-BC-index in non-epithelial cells (FIG. 5A). The majority of tissue samples contain substantial proportions of fat, as determined by the Epidish algorithm, and hence the inventors have plotted the WID-BC-index against the fat content of the respective tissue samples (FIG. 5B). Surprisingly the inventors find a very strong direct correlation between the WID-BC-index and the fat content of the sample irrespective of whether the sample was taken from an epithelial or non-epithelial organ. These findings strongly indicate that the WID-BC-index is reflective of a fat cell program.

### WID-BC-Index in Breast Tissue

We, and others, have demonstrated the existence of an epigenetic field defect in the normal breast adjacent to a breast cancer^{9, 23}. The inventors therefore wanted to assess whether the WID-BC-index, which was established in cervical smear samples in women with and without a breast cancer, is reflected in normal and cancerous breast tissue. The inventors analysed 14 normal breast samples from healthy women, 14 normal breast samples from women with a BRCA mutation, 14 normal breast samples adjacent to a triple-negative breast cancer, and 14 matched cancer samples. As expected, in contrast to cervical and buccal samples, the inventors found that fat cells constituted a substantial proportion of normal samples and substantially less so for cancerous breast tissue samples (FIG. 7H). The inventors also analysed breast tissue samples from a clinical trial consisting of 21 BRCA carriers and 23 healthy controls who donated samples before and after treatment with either mifepristone or placebo. As expected, the inventors found that in all four sample groups and the clinical trial samples the index is substantially higher in fat cells (FIG. 5C) and that this leads to an overall increase in the index in comparison to cervical samples. After linearly adjusting for fat content the inventors observed a distinct trend in which the WID-BC-index increased in tissues that are at increased risk of developing cancer and was highest in breast cancer (FIG. 5D).

eFORGE

In order to further assess the functional aspect of the WID-BC-index the inventors utilized the eFORGE tool 24 to search for enrichment of cell-type specific CpGs in the top 1,000 hyper- and hypo-methylated epithelial CpGs. The strongest enrichment in (i) hyper-methylated CpGs was for breast epithelial cell-specific CpGs and muscle, fibroblasts and mesenchymal cells (FIG. 5E) and in (ii) hypo-methylated CpGs for a foetal-like program with enrichment for stem cells and foetal and gastrointestinal differentiation (FIG. 5F). These findings suggest that in cervical smear samples from breast cancer cases the epigenome has undergone an epigenetic re-programming which may be reflective of a limited capacity for mammary epithelial cell differentiation and a shift towards mesenchymal and foetal/gastrointestinal programs. In addition, a gene set enrichment analysis was performed using the Broad Institute's Molecular Signatures Database 25 (Extended Data Table 2) and breast cancer associated pathways were enriched in hypo-methylated genes.

### WID-BC-Index Reflects Cervicovaginal Microbiome

Due to the fact that the WID-BC-index is reflective of an aberrant differentiation at the level of the epigenome the inventors wanted to assess whether this also impacts on functional aspects like the ability to host microbiome communities. The inventors analysed the cervicovaginal microbiomes from a subset of 229 women in the ovarian cancer dataset (56 controls and 173 ovarian cancer cases) which underwent 16S rRNA gene sequencing 26. Samples in which lactobacilli species accounted for greater than 50% of species present were classified as Type-L (95 samples), otherwise they were classified as Type-0 (134 samples). A principal component analysis of the 8,442 bimodal CpGs used in the WID-BC-index in samples with an IC<0.5 (48 Type-L and 61 Type-O) showed that the two bimodal subgroups overlap substantially with microbiome type (FIG. 5G). A similar pattern was observed in the epithelial component (FIG. 12A) but not the immune or shared components (FIG. 7B,C).

### Cancer Index Values and Clinical Actions

Four sub-groups defined by ranges of cancer index values are specified in Table 8 as corresponding to preferred clinical actions, comprising intensified screening, administration of therapeutics and surgery. The subgroups are quartiles based on control samples from the internal validation set. That is, these values of the index split the control samples into four equally sized groups. Odds ratio values are calculated by comparing the number of cases and controls in a given quartile to the first quartile (which is taken as a reference). Odds ratio values are determined for breast cancer risk, ovarian cancer risk and BRCA1 mutation risk. For the breast cancers these estimates are based on the internal validation dataset. For example, a woman in the fourth quartile is roughly 15 times more likely to have breast cancer than a woman in the first quartile.

### DISCUSSION

The inventors have identified a cervical smear based DNAme signature (the WID-BC-index) which provides an unprecedented opportunity to identify women with a primary breast cancer with poor prognosis features based on a bio-sample which has no direct (anatomical) link to the diseased organ (i.e. women in the top quartile of the WID-BC-Index have a 15.7 fold increased risk for breast cancer independent of any other risk factors; Table 5b). The fact that the WID-BC-index discovered in a cervical smear sample (i) does not increase with tumour size or surrogates for dissemination (i.e. nodal metastasis), (ii) increases in normal breast samples with increasing tendency towards cancer and is highest in the cancer tissue and (iii) is reflective of a fat cell epigenetic program, strongly supports the view that cervical DNAme reflects breast cancer predisposition rather than purely the current presence of an established breast cancer. This notion is further supported by the fact that the index also identifies women with ovarian cancer and those women who have a very high risk of developing both breast and ovarian cancer (i.e. due to a BRCA1 mutation)—likely at least in part due to cell-nonautonomous factors²⁷. The fact that the WID-BC-index is able to identify women who have a BRCA1 mutation is quite surprising because the inclusion criteria that the inventors have chosen for this set (i.e. women without risk reducing surgery and who had not developed a cancer yet) were biasing against those carriers with the highest risk.

Our findings described here are consistent with data published more than 30 years ago showing that patients with hereditary breast cancer and their first degree relatives harbour a differentiation defect²⁸. Interestingly this “epigenetic misprogramming” leads to functional alterations locally (i.e. reduced capacity to host lactobacilli in the vagina) and due to “epigenetic assimilation” of epithelial cells to fat cells might facilitate unregulated mixing of epithelial and fat cells in the breast, which is synonymous with invasive growth. The fact that the pathways associated with breast cancer metastasis into organs with high fat content (bone metastasis via the bone marrow) were enriched in hypo-methylated genes strongly supports this.

Considerable effort in the past has shown that by combining SNPs, mammographic density, and epidemiologic risk factors the AUC that predicts breast cancer can be increased up to 0.68⁶. Studying population-based cervical smear samples from women who develop a breast cancer several years after sample donations will be a prerequisite in order to assess whether the actual risk-predictive nature of the WID-BC-index will continue to outperform current breast cancer predictive algorithms. Whether DNAme profiles assessed in cervical smear samples, and/or in breast samples, can act as surrogates for monitoring breast cancer preventive measures will need to be assessed in prospective clinical trials.

TABLE 5A

| Quantile | Control | Cancer | OR (unadjusted) | | OR (adjusted) | |
|---|---|---|---|---|---|---|
| (−1.53, −0.58) | 75 | 2 | 1.00 | (reference) | 1.00 | (reference) |
| (−0.58, −0.28) | 74 | 5 | 2.42 | (0.48, 19.25) | 2.29 | (0.45, 17.15) |
| (−0.28, 0.07) | 74 | 17 | 8.01 | (2.17, 56.31) | 8.47 | (2.23, 55.81) |
| (0.07, 1.62) | 74 | 88 | 41.11 | (12.33, 274.77) | 41.73 | (12.2, 262.62) |

TABLE 5B

| | Con- | Can- | | | | |
|---|---|---|---|---|---|---|
| Quantile | trol | cer | OR (unadjusted) | | OR (adjusted) | |
| (−1.53, −0.58) | 58 | 8 | 1.00 | (reference) | 1.00 | (reference) |
| (−0.58, −0.28) | 69 | 8 | 0.84 | (0.29, 2.46) | 0.89 | (0.3, 2.67) |
| (−0.28, 0.07) | 50 | 14 | 2 | (0.78, 5.46) | 2.57 | (0.95, 7.51) |
| (0.07, 1.62) | 48 | 83 | 12.19 | (5.62, 29.86) | 15.67 | (6.59, 42.38) |

TABLE 5C

| | Con- | Can- | | | | |
|---|---|---|---|---|---|---|
| Quantile | trol | cer | OR (unadjusted) | | OR (adjusted) | |
| (−1.53, −0.58) | 75 | 13 | 1.00 | (reference) | 1.00 | (reference) |
| (−0.58, −0.28) | 74 | 11 | 0.86 | (0.35, 2.06) | 0.76 | (0.3, 1.94) |
| (−0.28, 0.07) | 74 | 17 | 1.32 | (0.6, 2.98) | 0.81 | (0.32, 2.03) |
| (0.07, 1.62) | 74 | 42 | 3.23 | (1.64, 6.76) | 2.42 | (1.18, 5.21) |

TABLE 5D

| | Con- | | | | | |
|---|---|---|---|---|---|---|
| Quantile | trol | Cases | OR (unadjusted) | | OR (adjusted) | |
| (−1.53, −0.58) | 54 | 17 | 1.00 | (reference) | 1.00 | (reference) |
| (−0.58, −0.28) | 20 | 14 | 2.2 | (0.91, 5.36) | 1.74 | (0.65, 4.6) |
| (−0.28, 0.07) | 20 | 10 | 1.59 | (0.6, 4.05) | 1.27 | (0.45, 3.46) |
| (0.07, 1.62) | 20 | 16 | 2.51 | (1.06, 6.01) | 2.26 | (0.78, 6.61) |

TABLE 5E

| | Con- | | | | | |
|---|---|---|---|---|---|---|
| Quantile | trol | Cases | OR (unadjusted) | | OR (adjusted) | |
| (−1.39, −1.02) | 29 | 4 | 1.00 | (reference) | 1.00 | (reference) |
| (−1.02, −0.75) | 29 | 14 | 3.37 | (1.05, 13.45) | 2.9 | (0.86, 11.64) |
| (−0.75, −0.45) | 29 | 17 | 4.08 | (1.3, 16.03) | 4.91 | (1.5, 19.52) |
| (−0.45, 0.91) | 29 | 27 | 6.44 | (2.16, 24.59) | 8.75 | (2.52, 38.02) |

TABLE 5F

| | Con- | | | | | |
|---|---|---|---|---|---|---|
| Quantile | trol | Cases | OR (unadjusted) | | OR (adjusted) | |
| (−2.09, −1.5) | 29 | 7 | 1.00 | (reference) | 1.00 | (reference) |
| (−1.5, −1.24) | 29 | 6 | 0.86 | (0.24, 2.97) | 0.84 | (0.24, 2.84) |
| (−1.24, −0.95) | 28 | 17 | 2.46 | (0.9, 7.33) | 2.71 | (0.99, 8.13) |
| (−0.95, 0.04) | 29 | 27 | 3.76 | (1.46, 10.77) | 4.63 | (1.68, 14.32) |

TABLE 6A
External validation

| Factor | Group | Control | | Cancer | | OR | | p-value |
|---|---|---|---|---|---|---|---|---|
| Age (years) | <52 | 91 | (40%) | 61 | (54%) | 1.00 | (reference) | — |
| | 52-64 | 85 | (38%) | 27 | (24%) | 0.48 | (0.27, 0.81) | 0.006 |
| | >64 | 49 | (22%) | 25 | (22%) | 0.76 | (0.42, 1.36) | 0.36 |
| Age at menarche (years) | <12 | 34 | (15%) | 31 | (27%) | 1.87 | (0.99, 3.54) | 0.05 |
| | Dec-13 | 119 | (53%) | 47 | (42%) | 0.81 | (0.48, 1.38) | 0.44 |
| | >13 | 72 | (32%) | 35 | (31%) | 1.00 | (reference) | — |
| Parous | Yes | 166 | (74%) | 81 | (72%) | 1.00 | (reference) | — |
| | No | 59 | (26%) | 32 | (28%) | 1.11 | (0.67, 1.84) | 0.68 |
| Age at birth of first live child | <21 | 16 | (10%) | 1 | (1%) | 1.00 | (reference) | — |
| (years) | 21-27 | 61 | (38%) | 20 | (25%) | 4.61 | (0.84, 116.32) | 0.08 |
| | >27 | 84 | (52%) | 59 | (74%) | 9.85 | (1.92, 241.73) | 0.003 |
| Menopausal status | Pre | 79 | (35%) | 57 | (50%) | 1.00 | (reference) | — |
| | Post | 146 | (65%) | 56 | (50%) | 0.53 | (0.34, 0.84) | 0.007 |
| Age at menopause (years) | <46 | 82 | (37%) | 36 | (33%) | 1.08 | (0.56, 2.13) | 0.81 |
| | 46-52 | 91 | (41%) | 53 | (49%) | 1.43 | (0.77, 2.75) | 0.26 |
| | >52 | 47 | (21%) | 19 | (18%) | 1.00 | (reference) | — |
| BMI (kg/m2) | <25 | 117 | (52%) | 67 | (59%) | 1.00 | (reference) | — |
| | 25-30 | 57 | (25%) | 29 | (26%) | 0.89 | (0.51, 1.52) | 0.67 |
| | >30 | 51 | (23%) | 17 | (15%) | 0.59 | (0.31, 1.08) | 0.09 |
| Oral contraceptive use | No | 29 | (34%) | 14 | (29%) | 1.00 | (reference) | — |
| (pre menopausal only) | Yes | 57 | (66%) | 35 | (71%) | 1.27 | (0.59, 2.79) | 0.55 |
| Hormone replacement therapy | No | 82 | (95%) | 49 | (98%) | 1.00 | (reference) | — |
| (post menopausal only) | Yes | 4 | (5%) | 1 | (2%) | 0.46 | (0.02, 3.46) | 0.49 |
| Smoking | No | 190 | (85%) | 100 | (88%) | 1.00 | (reference) | — |
| | Yes | 34 | (15%) | 13 | (12%) | 0.73 | (0.36, 1.42) | 0.37 |
| First degree relatives | 0 | 193 | (86%) | 98 | (87%) | 1.00 | (reference) | — |
| with breast cancer | 1 | 30 | (13%) | 14 | (12%) | 0.92 | (0.45, 1.8) | 0.82 |
| | 2+ | 2 | (1%) | 1 | (1%) | 1.05 | (0.03, 13.08) | 0.97 |

TABLE 6B
External validation

| Factor | Group | Control | | Cancer | | OR | | p-value |
|---|---|---|---|---|---|---|---|---|
| Age (years) | <52 | 538 | (62%) | 162 | (49%) | 1.00 | (reference) | — |
| | 52-64 | 208 | (24%) | 96 | (29%) | 1.53 | (1.13, 2.06) | 0.006 |
| | >64 | 123 | (14%) | 71 | (22%) | 1.92 | (1.36, 2.69) | <10\hat{ }−3 |
| Age at menarche (years) | <12 | 150 | (17%) | 67 | (21%) | 1.45 | (1, 2.12) | 0.05 |
| | Dec-13 | 437 | (51%) | 174 | (53%) | 1.3 | (0.96, 1.76) | 0.09 |
| | >13 | 277 | (32%) | 85 | (26%) | 1.00 | (reference) | — |
| Parous | Yes | 636 | (73%) | 263 | (80%) | 1.00 | (reference) | — |
| | No | 232 | (27%) | 65 | (20%) | 0.68 | (0.49, 0.92) | 0.01 |
| Age at birth of first live child | <21 | 22 | (3%) | 10 | (4%) | 1.00 | (reference) | — |
| (years) | 21-27 | 211 | (34%) | 83 | (32%) | 0.86 | (0.4, 1.98) | 0.71 |
| | >27 | 396 | (63%) | 167 | (64%) | 0.92 | (0.43, 2.09) | 0.83 |
| Menopausal status | Pre | 509 | (59%) | 159 | (48%) | 1.00 | (reference) | — |
| | Post | 360 | (41%) | 170 | (52%) | 1.51 | (1.17, 1.95) | 0.002 |
| Age at menopause (years) | <46 | 423 | (54%) | 105 | (34%) | 0.36 | (0.25, 0.53) | <10\hat{ }−6 |
| | 46-52 | 260 | (33%) | 138 | (44%) | 0.78 | (0.54, 1.13) | 0.18 |
| | >52 | 101 | (13%) | 69 | (22%) | 1.00 | (reference) | — |
| BMI (kg/m2) | <25 | 445 | (51%) | 201 | (61%) | 1.00 | (reference) | — |
| | 25-30 | 275 | (32%) | 81 | (25%) | 0.65 | (0.48, 0.88) | 0.005 |
| | >30 | 149 | (17%) | 47 | (14%) | 0.7 | (0.48, 1.01) | 0.05 |
| Oral contraceptive use | No | 378 | (84%) | 175 | (81%) | 1.00 | (reference) | — |
| (pre menopausal only) | Yes | 67 | (15%) | 36 | (17%) | 1.16 | (0.74, 1.8) | 0.51 |
| Hormone replacement therapy | No | 441 | (98%) | 216 | (100%) | 1.00 | (reference) | — |
| (post menopausal only) | Yes | 8 | (2%) | 1 | (0%) | 0.29 | (0.01, 1.61) | 0.18 |
| Smoking | No | 759 | (87%) | 290 | (88%) | 1.00 | (reference) | — |
| | Yes | 95 | (11%) | 39 | (12%) | 1.08 | (0.72, 1.59) | 0.72 |
| First degree relatives with | 0 | 770 | (89%) | 274 | (83%) | 1.00 | (reference) | — |
| breast cancer | 1 | 95 | (11%) | 54 | (16%) | 1.6 | (1.11, 2.29) | 0.01 |
| | 2+ | 4 | (0%) | 1 | (0%) | 0.77 | (0.03, 5.62) | 0.83 |

TABLE 6B
External validation

| Factor | Group | Set | |
|---|---|---|---|
| Age (years) | <52 | 61 | (54%) |
| | 52-64 | 27 | (24%) |
| | >64 | 25 | (22%) |
| Menopausal status | Pre | 57 | (50%) |
| | Post | 56 | (50%) |
| T-Stage | T1 | 50 | (44%) |
| | T2 | 49 | (43%) |
| | T3/T4 | 13 | (12%) |
| N-Stage | N0 | 49 | (43%) |
| | >N0 | 62 | (55%) |
| ER | Pos | 94 | (83%) |
| | Neg | 17 | (15%) |
| PR | Pos | 82 | (73%) |
| | Neg | 27 | (24%) |
| HER2 | Pos | 48 | (42%) |
| | Neg | 64 | (57%) |
| Grade | I | 8 | (7%) |
| | II | 51 | (45%) |
| | III | 54 | (48%) |
| Histology | Ductal | 93 | (82%) |
| | Lobular | 11 | (10%) |
| | Other | 9 | (8%) |

TABLE 6B
Internal validation

| Factor | Group | Set | |
|---|---|---|---|
| Age (years) | <52 | 162 | (49%) |
| | 52-64 | 96 | (29%) |
| | >64 | 71 | (22%) |
| Menopausal status | Pre | 159 | (48%) |
| | Post | 170 | (52%) |
| T-Stage | T1 | 141 | (43%) |
| | T2 | 163 | (50%) |
| | T3/T4 | 22 | (7%) |
| N-Stage | N0 | 153 | (47%) |
| | >N0 | 167 | (51%) |
| ER | Pos | 282 | (86%) |
| | Neg | 46 | (14%) |
| PR | Pos | 247 | (75%) |
| | Neg | 79 | (24%) |
| HER2 | Pos | 107 | (33%) |
| | Neg | 218 | (66%) |
| Grade | I | 21 | (6%) |
| | II | 135 | (41%) |
| | III | 170 | (52%) |
| Histology | Ductal | 242 | (74%) |
| | Lobular | 54 | (16%) |
| | Other | 33 | (10%) |

TABLE 7A

| | Con- | | | | | |
|---|---|---|---|---|---|---|
| Quantile | trol | Cases | OR (unadjusted) | | OR (adjusted) | |
| (−1.53, −0.58) | 54 | 21 | 1.00 | (reference) | 1.00 | (reference) |
| (−0.58, −0.28) | 20 | 13 | 1.67 | (0.69, 3.97) | 1.22 | (0.45, 3.17) |
| (−0.28, 0.07) | 20 | 9 | 1.16 | (0.44, 2.94) | 0.85 | (0.29, 2.3) |
| (0.07, 1.62) | 20 | 10 | 1.29 | (0.5, 3.2) | 1.14 | (0.38, 3.27) |

TABLE 7B

| | Con- | | | | | |
|---|---|---|---|---|---|---|
| Quantile | trol | Cases | OR (unadjusted) | | OR (adjusted) | |
| (−1.53, −0.58) | 54 | 21 | 1.00 | (reference) | 1.00 | (reference) |
| (−0.58, −0.28) | 20 | 13 | 1.67 | (0.69, 3.97) | 1.22 | (0.45, 3.17) |
| (−0.28, 0.07) | 20 | 9 | 1.16 | (0.44, 2.94) | 0.85 | (0.29, 2.3) |
| (0.07, 1.62) | 20 | 10 | 1.29 | (0.5, 3.2) | 1.14 | (0.38, 3.27) |

TABLE 7C

| | Con- | | | | | |
|---|---|---|---|---|---|---|
| Quantile | trol | Cases | OR (unadjusted) | | OR (adjusted) | |
| (−1.53, −0.58) | 54 | 21 | 1.00 | (reference) | 1.00 | (reference) |
| (−0.58, −0.28) | 20 | 13 | 1.67 | (0.69, 3.97) | 1.22 | (0.45, 3.17) |
| (−0.28, 0.07) | 20 | 9 | 1.16 | (0.44, 2.94) | 0.85 | (0.29, 2.3) |
| (0.07, 1.62) | 20 | 10 | 1.29 | (0.5, 3.2) | 1.14 | (0.38, 3.27) |

TABLE 8

| | | | BRCA1 | |
|---|---|---|---|---|
| WID-BC | Breast | Ovarian | mutation | |
| ranges | cancer risk | cancer risk | risk | Clinical Action |
| (−1.53, −0.58) | 1 | 1 | 1 | No mammography screening, no preventive measures. Repeat test in 2 years |
| | | | | time. |
| (−0.58, −0.28) | 0.89 | 0.86 | 2.2 | Testing for a BRCA1 or BRCA2 germline mutation - if negative only routine |
| | (0.3, 2.67) | (0.35, 2.06) | (0.91, 5.36) | mammography screening. Repeat test in 2 years time. |
| (−0.28, 0.07) | 2.57 | 1.32 | 1.59 | (1) Testing for a BRCA1 or BRCA2 germline mutation; (2) Intensified breast |
| | (0.95, 7.51) | (0.6, 2.98) | (0.6, 4.05) | cancer screening using breast MRI every other year; (3) To consider taking any |
| | | | | of the following risk preventive drugs: Denosumab, “selective estrogen |
| | | | | receptor modulators” (SERMs) and “selective progesterone receptor |
| | | | | modulators” (SPRMs). SERMs may include Anordin, Bazedoxifene, |
| | | | | Broparestrol, Clomifene, Cyclofenil, Lasofoxifene, Ormeloxifene, Ospemifene, |
| | | | | Raloxifene, Tamoxifen. Preferably, the SERMs include Tamoxifen, |
| | | | | Bazedoxifene and Raloxifene. Preferably, the SPRMs include Mifepristone, |
| | | | | Ulipristal, Asoprisnil, Proellex, Onapristone, Asoprisnil and Lonaprisan. (6) |
| | | | | Repeat test annually. |
| (0.07, 1.62) | 15.67 | 3.23 | 2.51 | (1) Testing for a BRCA1 or BRCA2 germline mutation; (2) Intensified breast |
| | (6.59, 42.38) | (1.64, 6.76) | (1.06, 6.01) | cancer screening: using annual breast MRI and mammography; (3) To consider |
| | | | | taking any of the following risk preventive drugs: Denosumab, “selective |
| | | | | estrogen receptor modulators” (SERMs) and “selective progesterone receptor |
| | | | | modulators” (SPRMs). SERMs may include Anordin, Bazedoxifene, |
| | | | | Broparestrol, Clomifene, Cyclofenil, Lasofoxifene, Ormeloxifene, Ospemifene, |
| | | | | Raloxifene, Tamoxifen. Preferably, the SERMs include Tamoxifen, |
| | | | | Bazedoxifene and Raloxifene. Preferably, the SPRMs include Mifepristone, |
| | | | | Ulipristal, Asoprisnil, Proellex, Onapristone, Asoprisnil and Lonaprisan. (4) To |
| | | | | consider risk reducing blateral mastectomy +/− bilateral salpingoophorectomy. |
| | | | | (5) To consider early detection of ovarian cancer using a combination of |
| | | | | CA125 tumour marker and cancer-specific cell-free DNA methylation in |
| | | | | plasma/serum. (6) Repeat test annually. |

1. A method comprising assaying from a sample from an individual
the methylation status of a panel of one or more CpGs selected from a panel of CpGs identified at nucleotide positions 61 to 62 in SEQ ID NOs 1 to 29,000.

2. A method according to claim 1, wherein:
i) the panel of one or more CpGs comprises at least 500 CpGs selected from the CpGs identified at nucleotide positions 61 to 62 in SEQ ID NOs 1 to 29,000, and optionally wherein the panel of one or more CpGs comprises at least the CpGs identified in SEQ ID NOs 1 to 500 and identified at nucleotide positions 61 to 62; or
ii) the panel of one or more CpGs comprises at least 1000 CpGs selected from the CpGs identified at nucleotide positions 61 to 62 in SEQ ID NOs 1 to 29,000, and optionally wherein the panel of one or more CpGs comprises at least the CpGs identified in SEQ ID NOs 1 to 1000 and identified at nucleotide positions 61 to 62; or
iii) wherein the panel of one or more CpGs comprises at least 2000 CpGs selected from the CpGs identified at nucleotide positions 61 to 62 in SEQ ID NOs 1 to 29,000, and optionally wherein the panel of one or more CpGs comprises at least the CpGs identified in SEQ ID NOs 1 to 2000 and identified at nucleotide positions 61 to 62; or
iv) wherein the panel of one or more CpGs comprises at least 10,000 CpGs selected from the CpGs identified at nucleotide positions 61 to 62 in SEQ ID NOs 1 to 29,000, and optionally wherein the panel of one or more CpGs comprises at least the CpGs identified in SEQ ID NOs 1 to 10,000 and identified at nucleotide positions 61 to 62; or
v) wherein the panel of one or more CpGs comprises at least the 29,000 CpGs identified at nucleotide positions 61 to 62 in SEQ ID NOs 1 to 29,000.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A method according to claim 1, wherein the step of assaying the methylation status of each CpG in the panel of one or more CpGs comprises:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

19. A method according to claim 1, wherein the step of assaying the methylation status of each CpG in the panel of one or more CpGs comprises:
a. bisulphite converting the DNA; or
b. performing the steps of oxidising 5-methyl cytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxyl cytosine bases (5caC) to dihydrouracil bases (DHU), optionally with pyridine borane.

20. A method according to claim 1, wherein the method further comprises:
a. assaying from the sample from the individual the proportion of epithelial cells;
b. assaying from the sample from the individual the proportion of fat cells; and/or
c. assaying from the sample from the individual differentiation characteristics of non-fat cells, optionally wherein assaying the proportion of epithelial and/or fat cells and/or assaying differentiation characteristics of non-fat cells comprises performing a method comprising:
a. gene expression profiling;
b. non-coding RNA-profiling;
c. epigenome profiling;
d. DNA methylation profiling;
e. deriving a WID-BC-Index; and/or
f. immunohistochemistry; and
arriving at a determination based on the results of the method.

21. (canceled)

22. A method of treating or preventing cancer in an individual, the method comprising administering one or more treatments to the individual determined to have cancer by the step of performing the method of claim 1 on a sample from the individual.

23.-50. (canceled)

51. A method according to claim 22, further defined as comprising a step of stratifying the individual according to their cancer risk or of having cancer.

52. A method according to claim 51, wherein the individual is stratified as not having cancer or as having a low risk of cancer development, and wherein the individual is subjected to one or more treatments according to their stratification, wherein the one or more treatments comprise intensified screening, preferably wherein the intensified screening comprises any one or more of:
a. a test for a BRCA1 and/or BRCA2 germline mutation;
b. a repeat method according to claim 1, preferably wherein the repeat method is about two years after the previous method,
optionally wherein when the test for a BRCA1 and/or BRCA2 germline mutation is positive, the intensified screening further comprises a mammogram, preferably wherein when the test for a BRCA1 and/or BRCA2 germline mutation is negative, the individual is subjected to routine mammography screening, and more preferably wherein the routine screening comprises a mammogram about three years following the test for a BRCA1 and/or BRCA2 germline mutation.

53. A method according to claim 51, wherein the individual is stratified as having a moderate risk of having cancer or as having a moderate risk of cancer development, and wherein the individual is subjected to one or more treatments according to their stratification, wherein the one or more treatments comprise any of:
a. intensified screening, preferably wherein the intensified screening comprises one or more of:
i. a test for a BRCA1 and/or BRCA2 germline mutation;
ii. a breast MRI scan, preferably wherein the scan is repeated about two years after the previous scan;
iii. a repeat assay according to any one of claims 1 to 20, preferably wherein the repeat assay is performed about one year after the previous assay;
b. administration of one or more of Denosumab, “selective estrogen receptor modulators” (SERMs), optionally comprising Anordin, Bazedoxifene, Broparestrol, Broparestrol, Clomifene, Cyclofenil, Lasofoxifene, Ormeloxifene, Ospemifene, Raloxifene, Tamoxifen, preferably wherein the SERMs comprise Tamoxifen, Bazedoxifene and Raloxifene, and “selective progesterone receptor modulators” (SPRMs), optionally comprising Mifepristone, Ulipristal, Asoprisnil, Proellex, Onapristone, Asoprisnil and Lonaprisan.

54. A method according to claim 51, wherein the individual is stratified as having cancer or as having a high risk of cancer development, and wherein the individual is subjected to one or more treatments according to their stratification, wherein the one or more treatments comprise any of:
a. intensified screening, preferably wherein the intensified screening comprises one or more of:
i. a test for a BRCA1 and/or BRCA2 germline mutation;
ii. a breast MRI scan, preferably wherein the scan is repeated about a year after the previous scan;
iii. a mammogram, preferably wherein the mammogram is repeated about a year after the previous mammogram;
iv. a test for CA125, preferably wherein the test is repeated three-monthly;
v. a test for cell-free tumour DNA methylation in plasma/serum, preferably wherein the test is repeated annually;
vi. a test for cell-free tumour DNA methylation in vaginal fluid, preferably wherein the test is repeated annually;
vii. a repeat method according to any claim 1, preferably wherein the repeat method is performed about one year after the previous method;
b. administration of one or more of Denosumab, “selective estrogen receptor modulators” (SERMs), optionally comprising Anordin, Bazedoxifene, Broparestrol, Broparestrol, Clomifene, Cyclofenil, Lasofoxifene, Ormeloxifene, Ospemifene, Raloxifene, Tamoxifen, preferably wherein the SERMs comprise Tamoxifen, Bazedoxifene and Raloxifene, and “selective progesterone receptor modulators” (SPRMs), optionally comprising Mifepristone, Ulipristal, Asoprisnil, Proellex, Onapristone, Asoprisnil and Lonaprisan;
c. a bilateral mastectomy; and/or
d. a bilateral salpingo-oophorectomy.

55. A method according to 22, wherein the one or more treatments that the individual is subjected to are repeated on a monthly, three monthly, six monthly, yearly or two yearly basis following an initial administration.

56. A method of assaying methylation in an individual at multiple time points, the method comprising: (a) performing the method according to claim 1 at a first time point; (b) performing the method according to claim 1 at one or more further time points; and (c) detecting differential methylation status between (a) and (b).

57. A method according to claim 56, wherein the further time points are monthly, three monthly, six monthly, yearly or two yearly basis following an initial assessment; and/or
wherein one or more treatments are administered to the individual according to claim 10.

58. A method according to claim 56, wherein the method further comprises:
a. assaying the proportion of epithelial cells in the sample from the individual between any two or more time points and assessing if the proportion changes between time points;
b. assaying the proportion of fat cells in the sample from the individual between any two or more time points and assessing if the proportion changes between time points; and/or
c. assaying differentiation characteristics of non-fat cells in the sample from the individual between any two or more time points and assessing if the proportion changes between time points.

59. An assay according to claim 1, wherein the sample is obtained from a tissue comprising epithelial cells, preferably wherein the sample is not obtained from breast or ovarian tissue, optionally wherein the sample is obtained from:
a. cervical tissue;
b. vaginal tissue;
c. cervicovaginal tissue;
d. buccal tissue; and/or
e. breast tissue;
preferably wherein the sample is obtained from cervical tissue, most preferably wherein the sample is obtained from tissue from a cervical smear.

60. An array for discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel comprises at least 500 CpGs selected from the CpGs identified at nucleotide position 61 to 62 in SEQ ID NOs 1 to 29,000.

61. An array according to claim 60, provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less; and/or wherein the panel comprises one or more CpGs selected from the CpGs identified at nucleotide positions 61 to 62 in SEQ ID NOs 1 to 29,000; and/or wherein the array further comprises one or more oligonucleotides comprising any set of CpGs selected from the CpGs identified at nucleotide positions 61 to 62 in SEQ ID NOs 1 to 29,000, wherein the one or more oligonucleotides are hybridized to corresponding oligonucleotide probes of the array.

62. A hybridized array, wherein the array is obtainable by hybridizing to an array according to claim 60 a group of oligonucleotides comprising any set of CpGs selected from the CpGs identified at nucleotide positions 61 to 62 in SEQ ID NOs 1 to 29,000.

63. A process for making the hybridized array according to claim 62, comprising contacting an array comprising oligonucleotide probes specific for a methylated form of each CpG in a CpG panel and oligonucleotide probes specific for a non-methylated form of each CpG in the panel; wherein the panel comprises at least 500 CpGs selected from the CpGs identified at nucleotide position 61 to 62 in SEQ ID NOs 1 to 29,000 with a group of oligonucleotides comprising any set of CpGs selected from the CpGs identified at nucleotide positions 61 to 62 in SEQ ID NOs 1 to 29,000.
